SEQUENCE LISTING (276 sequences)

SEQ ID NO: 1 - DNA, 25 bp, Artificial Sequence
  forward primer
caccgcatatgaaataccaaacacg

SEQ ID NO: 2 - DNA, 25 bp, Artificial Sequence
  reverse primer
aaaccgtgtttggtatttcatatgc

SEQ ID NO: 3 - DNA, 25 bp, Artificial Sequence
  forward primer
caccgcattgctgtatacaatcatg

SEQ ID NO: 4 - DNA, 25 bp, Artificial Sequence
  reverse primer
aaaccatgattgtatacagcaatgc

SEQ ID NO: 5 - DNA, 25 bp, Artificial Sequence
  forward primer
caccgtgagtggagaagcacacacg

SEQ ID NO: 6 - DNA, 25 bp, Artificial Sequence
  reverse primer
aaaccgtgtgtgcttctccactcac

SEQ ID NO: 7 - DNA, 36 bp, Artificial sequence
  Description of sequence Forward primer D243E
tccaagaagaggattttaataacctcaaccaattac

SEQ ID NO: 8 - DNA, 34 bp, Artificial sequence
  Description of sequence Reverse primer D243E
gttattaaaatcctcttcttggatttttgcaatc

SEQ ID NO: 9 - DNA, 29 bp, Artificial sequence
  Description of sequence Forward primer D244Y
ccaagaagattattttaataacctcaacc

SEQ ID NO: 10 - DNA, 34 bp, Artificial sequence
  Description of sequence Reverse primer D244Y
gttattaaaataatcttcttggatttttgcaatc

SEQ ID NO: 11 - DNA, 28 bp, Artificial sequence
  Description of sequence Forward primer E617A
ggaccatggcgagtgagtctcttagaac

SEQ ID NO: 12 - DNA, 29 bp, Artificial sequence
  Description of sequence Reverse primer E617A
agactcactcgccatggtcctgctggtgg

SEQ ID NO: 13 - DNA, 40 bp, Artificial sequence
  Description of sequence Forward primer E654K
taaaattagagaaattagacatctctaaaaattccctaag

SEQ ID NO: 14 - DNA, 39 bp, Artificial sequence
  Description of sequence Reverse primer E654K
gtctaatttctctaattttagcagattcttgaataattg

SEQ ID NO: 15 - DNA, 35 bp, Artificial sequence
  Description of sequence Forward primer F310L
ccccaagatggcttaagaacatcaacaaactccag

SEQ ID NO: 16 - DNA, 33 bp, Artificial sequence
  Description of sequence Reverse primer F310L
gatgttcttaagccatcttgggggcacatgctg

SEQ ID NO: 17 - DNA, 35 bp, Artificial sequence
  Description of sequence Forward primer H630Y
cagaggaaattacttagatgttttatggagagaag

SEQ ID NO: 18 - DNA, 34 bp, Artificial sequence
  Description of sequence Reverse primer H630Y
acatctaagtaatttcctctgaattccagagttc

SEQ ID NO: 19 - DNA, 25 bp, Artificial sequence
  Description of sequence Forward primer H781L
gcttttgcttcataatcggtttctg

SEQ ID NO: 20 - DNA, 26 bp, Artificial sequence
  Description of sequence Reverse primer H781L
gattatgaagcaaaagcaacatcttc

SEQ ID NO: 21 - DNA, 37 bp, Artificial sequence
  Description of sequence Forward primer I505T
aaatagtacattttttgtcaagtcctctgattttcag

SEQ ID NO: 22 - DNA, 37 bp, Artificial sequence
  Description of sequence Reverse primer I505T
caaaaaatgtactatttttacttagatccaaggtctg

SEQ ID NO: 23 - DNA, 40 bp, Artificial sequence
  Description of sequence Forward primer I657T
gaggaattagacacctctaaaaattccctaagtttcttgc

SEQ ID NO: 24 - DNA, 42 bp, Artificial sequence
  Description of sequence Reverse primer I657T
gaatttttagaggtgtctaattcctctaattttagcagattc

SEQ ID NO: 25 - DNA, 33 bp, Artificial sequence
  Description of sequence Forward primer K684*
ctctttggcctaaaatgggctcaaatctttcag

SEQ ID NO: 26 - DNA, 39 bp, Artificial sequence
  Description of sequence Reverse primer K684*
gagcccattttaggccaaagagagattctttagatttgg

SEQ ID NO: 27 - DNA, 30 bp, Artificial sequence
  Description of sequence Forward primer L134P
ccctttacccggatggaaaccagctactag

SEQ ID NO: 28 - DNA, 33 bp, Artificial sequence
  Description of sequence Reverse primer L134P
ggtttccatccgggtaaagggattttaaataag

SEQ ID NO: 29 - DNA, 33 bp, Artificial sequence
  Description of sequence Forward primer L227fs*
gccatctacttaacagaactatatctctacaac

SEQ ID NO: 30 - DNA, 36 bp, Artificial sequence
  Description of sequence Reverse primer L227fs*
gatatagttctgttaagtagatggcaaaacagtagg

SEQ ID NO: 31 - DNA, 25 bp, Artificial sequence
  Description of sequence Forward primer L988S
gattatctcgatatttcttgagaag

SEQ ID NO: 32 - DNA, 25 bp, Artificial sequence
  Description of sequence Reverse primer L988S
gaaatatcgagataatcacatcaac

SEQ ID NO: 33 - DNA, 31 bp, Artificial sequence
  Description of sequence Forward primer N275T
ccgtgtaaaactaattctcccctacagatcc

SEQ ID NO: 34 - DNA, 35 bp, Artificial sequence
  Description of sequence Reverse primer N275T
ggagaattagttttacacggcgcacaaggaaatgg

SEQ ID NO: 35 - DNA, 34 bp, Artificial sequence
  Description of sequence Forward primer N158Tfs*11
ctcagccttgaggcaacaacatcttttccatcag

SEQ ID NO: 36 - DNA, 30 bp, Artificial sequence
  Description of sequence Reverse primer N158Tfs*11
agatgttgttgcctcaaggctgagaagctg

SEQ ID NO: 37 - DNA, 33 bp, Artificial sequence
  Description of sequence Forward primer P715S
gaccactgtctctgagagattatccaactgttc

SEQ ID NO: 38 - DNA, 35 bp, Artificial sequence
  Description of sequence Reverse primer P715S
gataatctctcagagacagtggtcagttggttgtg

SEQ ID NO: 39 - DNA, 26 bp, Artificial sequence
  Description of sequence Forward primer A819T
gccaggaacacacaagggccaaagtg

SEQ ID NO: 40 - DNA, 26 bp, Artificial sequence
  Description of sequence Reverse primer A819T
cttgtgtgttcctggccccacacaag

SEQ ID NO: 41 - DNA, 35 bp, Artificial sequence
  Description of sequence Forward primer Q710Rfs*
cagccacaaccgaccactgtccctgagagattatc

SEQ ID NO: 42 - DNA, 31 bp, Artificial sequence
  Description of sequence Reverse primer Q710Rfs*
gacagtggtcggttgtggctgaggtccaaag

SEQ ID NO: 43 - DNA, 32 bp, Artificial sequence
  Description of sequence Forward primer V795F
ctgtgtggtttttctggtgggttaaccatacg

SEQ ID NO: 44 - DNA, 33 bp, Artificial sequence
  Description of sequence Reverse primer V795F
acccaccagaaaaaccacacagcatcacaggtg

SEQ ID NO: 45 - DNA, 30 bp, Artificial sequence
  Description of sequence Forward primer S301P
cagtaaccctcttcagcatgtgcccccaag

SEQ ID NO: 46 - DNA, 40 bp, Artificial sequence
  Description of sequence Reverse primer S301P
gctgaagagggttactgtgtagacgtaaaacttttaattc

SEQ ID NO: 47 - DNA, 37 bp, Artificial sequence
  Description of sequence Forward primer A288V
atgcttttgatgtgctgacagaattaaaagttttacg

SEQ ID NO: 48 - DNA, 34 bp, Artificial sequence
  Description of sequence Reverse primer A288V
ttctgtcagcacatcaaaagcatttacagggatc

SEQ ID NO: 49 - DNA, 33 bp, Artificial sequence
  Description of sequence Forward primer V219I
tgtcacagccatccctactgttttgccatctac

SEQ ID NO: 50 - DNA, 34 bp, Artificial sequence
  Description of sequence Reverse primer V219I
cagtagggatggctgtgacattgttatctttcag

SEQ ID NO: 51 - DNA, 27 bp, Artificial sequence
  Description of sequence Forward primer A448V
ctcaaatgtcagaacttctgtagaaag

SEQ ID NO: 52 - DNA, 28 bp, Artificial sequence
  Description of sequence Reverse primer A448V
aagttctgacatttgagcagaagccaac

SEQ ID NO: 53 - DNA, 35 bp, Artificial sequence
  Description of sequence Forward primer R920K
agacccaaaagagaaacattttaatttatgtctcg

SEQ ID NO: 54 - DNA, 29 bp, Artificial sequence
  Description of sequence Reverse primer R920K
ttctcttttgggtcttccagtttggccac

SEQ ID NO: 55 - DNA, 36 bp, Artificial sequence
  Description of sequence Forward primer A1032T
gtctaaagaacaccctggccacagacaatcatgtgg

SEQ ID NO: 56 - DNA, 35 bp, Artificial sequence
  Description of sequence Reverse primer A1032T
ctgtggccagggtgttctttagacactgccagaag

SEQ ID NO: 57 - DNA, 35 bp, Artificial sequence
  Description of sequence Forward primer Q11L
gaagagactaattcttatcctttttaacataatcc

SEQ ID NO: 58 - DNA, 31 bp, Artificial sequence
  Description of sequence Reverse primer Q11L
taagaattagtctcttcagtgtccacattgg

SEQ ID NO: 59 - DNA, 33 bp, Artificial sequence; identical to SEQ ID NO: 49
  Description of sequence Forward primer V219I

SEQ ID NO: 60 - DNA, 34 bp, Artificial sequence; identical to SEQ ID NO: 50
  Description of sequence Reverse primer V219I

SEQ ID NO: 61 - DNA, 27 bp, Artificial sequence
  Description of sequence Forward primer A448V
ctcaaatgtcagaacttctgtagaaag

SEQ ID NO: 62 - DNA, 28 bp, Artificial sequence
  Description of sequence Reverse primer A448V
aagttctgacatttgagcagaagccaac

SEQ ID NO: 63 - DNA, 36 bp, Artificial sequence
  Description of sequence Forward primer V222D
ccctactgatttgccatctactttaacagaactata

SEQ ID NO: 64 - DNA, 34 bp, Artificial sequence
  Description of sequence Reverse primer V222D
agatggcaaatcagtagggacggctgtgacattg

SEQ ID NO: 65 - DNA, 36 bp, Artificial sequence; identical to SEQ ID NO: 55
  Description of sequence Forward primer A1032T

SEQ ID NO: 66 - DNA, 35 bp, Artificial sequence; identical to SEQ ID NO: 56
  Description of sequence Reverse primer A1032T

SEQ ID NO: 67 - DNA, 35 bp, Artificial sequence; identical to SEQ ID NO: 53
  Description of sequence Forward primer R920K

SEQ ID NO: 68 - DNA, 29 bp, Artificial sequence
  Description of sequence Reverse primer R920K
ttctcttttgggtcttccagtttggccac

SEQ ID NO: 69 - DNA, 35 bp, Artificial sequence
  Description of sequence Forward primer V92I
gaccatctgatagagatcgatttcagatgcaactg

SEQ ID NO: 70 - DNA, 32 bp, Artificial sequence
  Description of sequence Reverse primer V92I
gatctctatcagatggtccagtctgtgaaagg

SEQ ID NO: 71 - DNA, 35 bp, Artificial sequence
  Description of sequence Forward primer I759V
aataaagtccagatgatccaaaagaccagcttccc

SEQ ID NO: 72 - DNA, 34 bp, Artificial sequence
  Description of sequence Reverse primer I759V
tctggactttatttgagctgagatccagatatcg

SEQ ID NO: 73 - DNA, 34 bp, Artificial sequence
  Description of sequence Forward primer M236L
ctctacaacaacttgattgcaaaaatccaagaag

SEQ ID NO: 74 - DNA, 31 bp, Artificial sequence
  Description of sequence Reverse primer M236L
gcaatcaagttgttgtagagatatagttctg

SEQ ID NO: 75 - DNA, 35 bp, Artificial sequence
  Description of sequence Forward primer N350S
ctgtctttcagttttgaacttcaggtctatcgtgc

SEQ ID NO: 76 - DNA, 36 bp, Artificial sequence
  Description of sequence Reverse primer N350S
gacctgaagttcaaaactgaaagacagatccaattg

SEQ ID NO: 77 - DNA, 38 bp, Artificial sequence
  Description of sequence Forward primer K293E
ctgacagaattagaagttttacgtctacacagtaactc

SEQ ID NO: 78 - DNA, 34 bp, Artificial sequence
  Description of sequence Reverse primer K293E
gtagacgtaaaacttctaattctgtcagcgcatc

SEQ ID NO: 79 - DNA, 38 bp, Artificial sequence
  Description of sequence Forward primer T127I
tagtggactcatttatttaaaatccctttacctggatg

SEQ ID NO: 80 - DNA, 37 bp, Artificial sequence
  Description of sequence Reverse primer T127I
taaataaatgagtccactaaagcttctgggtttaatc

SEQ ID NO: 81 - DNA, 21 bp, Artificial sequence
  Description of sequence Forward primer I142M
ctagagatgccgcagggcctc

SEQ ID NO: 82 - DNA, 30 bp, Artificial sequence
  Description of sequence Reverse primer I142M
ctgcggcatctctagtagctggtttccatc

SEQ ID NO: 83 - DNA, 23 bp, Artificial sequence
  Description of sequence Forward primer L146F
gcagggcttcccgcctagcttac

SEQ ID NO: 84 - DNA, 28 bp, Artificial sequence
  Description of sequence Reverse primer L146F
ctaggcgggaagccctgcggtatctcta

SEQ ID NO: 85 - DNA, 32 bp, Artificial sequence
  Description of sequence Forward primer A83V
ctccccagtgtcctttcacagactggaccatc

SEQ ID NO: 86 - DNA, 30 bp, Artificial sequence
  Description of sequence Reverse primer A83V
aaggacactggggagatgtctggtatgtgg

SEQ ID NO: 87 - DNA, 25 bp, Artificial sequence
  Description of sequence Forward primer L988S
gattatctcgatatttcttgagaag

SEQ ID NO: 88 - DNA, 25 bp, Artificial sequence
  Description of sequence Reverse primer L988S
gaaatatcgagataatcacatcaac

SEQ ID NO: 89 - DNA, 34 bp, Artificial sequence
  Description of sequence Forward primer V222L
gccgtccctactcttttgccatctactttaacag

SEQ ID NO: 90 - DNA, 27 bp, Artificial sequence
  Description of sequence Reverse primer V222L
gatggcaaaagagtagggacggctgtg

SEQ ID NO: 91 - DNA, 30 bp, Artificial sequence
  Description of sequence Forward primer H86L
cgtcctttctcagactggaccatctggtag

SEQ ID NO: 92 - DNA, 25 bp, Artificial sequence
  Description of sequence Reverse primer H86L
cagtctgagaaaggacgctggggag

SEQ ID NO: 93 - DNA, 30 bp, Artificial sequence
  Description of sequence Forward primer V283A
cagatccctgcaaatgcttttgatgcgctg

SEQ ID NO: 94 - DNA, 26 bp, Artificial sequence
  Description of sequence Reverse primer V283A
agcatttgcagggatctgtaggggag

SEQ ID NO: 95 - DNA, 31 bp, Artificial sequence
  Description of sequence Forward primer R883H
gtatcagcatctaatatcaccagactgttgc

SEQ ID NO: 96 - DNA, 35 bp, Artificial sequence
  Description of sequence Reverse primer R883H
ctggtgatattagatgctgatacccctttatcttg

SEQ ID NO: 97 - DNA, 32 bp, Artificial sequence
  Description of sequence Forward primer H55Q
cacagacaagcaattgacagaaattcctggag

SEQ ID NO: 98 - DNA, 28 bp, Artificial sequence
  Description of sequence Reverse primer H55Q
ctgtcaattgcttgtctgtgcagtccac

SEQ ID NO: 99 - DNA, 28 bp, Artificial sequence
  Description of sequence Forward primer E617A
ggaccatggcgagtgagtctcttagaac

SEQ ID NO: 100 - DNA, 29 bp, Artificial sequence
  Description of sequence Reverse primer E617A
agactcactcgccatggtcctgctggtgg

SEQ ID NO: 101 - DNA, 33 bp, Artificial sequence
  Description of sequence Forward primer Q117K
caagaggctgaagattaaacccagaagctttag

SEQ ID NO: 102 - DNA, 32 bp, Artificial sequence
  Description of sequence Reverse primer Q117K
gtttaatcttcagcctcttgatgcacatgttg

SEQ ID NO: 103 - DNA, 32 bp, Artificial sequence
  Description of sequence Forward primer T858K
gtgatgatgaaagcaagtcacctctatttctg

SEQ ID NO: 104 - DNA, 31 bp, Artificial sequence
  Description of sequence Reverse primer T858K
gacttgctttcatcatcaccatgagaaagag

SEQ ID NO: 105 - DNA, 33 bp, Artificial sequence
  Description of sequence Forward primer N720S
ttatccagctgttccagaagcctcaagaatctg

SEQ ID NO: 106 - DNA, 33 bp, Artificial sequence
  Description of sequence Reverse primer N720S
gaacagctggataatctctcagggacagtggtc

SEQ ID NO: 107 - DNA, 36 bp, Artificial sequence
  Description of sequence Forward primer S582L
gccattattttcaattagaaggaattactcatatgc

SEQ ID NO: 108 - DNA, 39 bp, Artificial sequence
  Description of sequence Reverse primer S582L
gagtaattccttctaattgaaaataatggctattactgc

SEQ ID NO: 109 - DNA, 36 bp, Artificial sequence
  Description of sequence Forward primer A482T
caaagagacttctttcatgtctgttaatgaaagctg

SEQ ID NO: 110 - DNA, 32 bp, Artificial sequence
  Description of sequence Reverse primer A482T
catgaaagaagtctctttgtttttgaatctgc

SEQ ID NO: 111 - DNA, 32 bp, Artificial sequence
  Description of sequence Forward primer D438V
cttcaggagtttcaagtgaagttggcttctgc

SEQ ID NO: 112 - DNA, 40 bp, Artificial sequence
  Description of sequence Reverse primer D438V
gccaacttcacttgaaactcctgaaggtgatattttattc

SEQ ID NO: 113 - DNA, 33 bp, Artificial sequence
  Description of sequence Forward primer S663R
ctaaaaattccctaaggttcttgccttctggag

SEQ ID NO: 114 - DNA, 37 bp, Artificial sequence
  Description of sequence Reverse primer S663R
cagaaggcaagaaccttagggaatttttagagatgtc

SEQ ID NO: 115 - DNA, 31 bp, Artificial sequence
  Description of sequence Forward primer F388L
gagttgaaaagccttaacctctcgccattac

SEQ ID NO: 116 - DNA, 33 bp, Artificial sequence
  Description of sequence Reverse primer F388L
cgagaggttaaggcttttcaactctttaaagac

SEQ ID NO: 117 - DNA, 29 bp, Artificial sequence
  Description of sequence Forward primer T221del
gtcacagccgtccctgttttgccatctac

SEQ ID NO: 118 - DNA, 35 bp, Artificial sequence
  Description of sequence Reverse primer T221del
gatggcaaaacagggacggctgtgacattgttatc

SEQ ID NO: 119 - DNA, 31 bp, Artificial sequence
  Description of sequence Forward primer L557F
cggcttgatttattccattcaacagcatttg

SEQ ID NO: 120 - DNA, 33 bp, Artificial sequence
  Description of sequence Reverse primer L557F
gttgaatggaataaatcaagccggttgttggag

SEQ ID NO: 121 - DNA, 25 bp, Artificial sequence
  Description of sequence Forward primer I113F
caacatgtgcttcaagaggctgcag

SEQ ID NO: 122 - DNA, 29 bp, Artificial sequence
  Description of sequence Reverse primer I113F
gcctcttgaagcacatgttgttttttgac

SEQ ID NO: 123 - DNA, 33 bp, Artificial sequence
  Description of sequence Forward primer D332G
gaaattgggggtgctaaatttctgcattttctc

SEQ ID NO: 124 - DNA, 32 bp, Artificial sequence
  Description of sequence Reverse primer D332G
atttagcacccccaatttctttggccaagaag

SEQ ID NO: 125 - DNA, 27 bp, Artificial sequence
  Description of sequence Forward primer K993T
cttgagacgccctttcagaagtccaag

SEQ ID NO: 126 - DNA, 33 bp, Artificial sequence
  Description of sequence Reverse primer K993T
gacttctgaaagggcgtctcaagaaatatcaag

SEQ ID NO: 127 - DNA, 26 bp, Artificial sequence
  Description of sequence Forward primer L1001I
caagttcatccagctccggaaaaggc

SEQ ID NO: 128 - DNA, 30 bp, Artificial sequence
  Description of sequence Reverse primer L1001I
cggagctggatgaacttggacttctgaaag

SEQ ID NO: 129 - DNA, 34 bp, Artificial sequence
  Description of sequence Forward primer R357C
caggtctattgtgcatctatgaatctatcacaag

SEQ ID NO: 130 - DNA, 38 bp, Artificial sequence
  Description of sequence Reverse primer R357C
gattcatagatgcacaatagacctgaagttcaaaattg

SEQ ID NO: 131 - DNA, 30 bp, Artificial sequence
  Description of sequence Forward primer I174M
ctggccaacatggaaatactctacctgggc

SEQ ID NO: 132 - DNA, 37 bp, Artificial sequence
  Description of sequence Reverse primer I174M
gtagagtatttccatgttggccagttctgttagattc

SEQ ID NO: 133 - DNA, 30 bp, Artificial sequence
  Description of sequence Forward primer E929Q
tatgtctccaggaaagggactggttaccag

SEQ ID NO: 134 - DNA, 38 bp, Artificial sequence
  Description of sequence Reverse primer E929Q
cagtccctttcctggagacataaattaaaatgtttctc

SEQ ID NO: 135 - DNA, 32 bp, Artificial sequence
  Description of sequence Forward primer R750Q
gccttccagttgcaatatctggatctcagctc

SEQ ID NO: 136 - DNA, 31 bp, Artificial sequence
  Description of sequence Reverse primer R750Q
gatccagatattgcaactggaaggcatcttg

SEQ ID NO: 137 - DNA, 35 bp, Artificial sequence
  Description of sequence Forward primer R357H
caggtctatcatgcatctatgaatctatcacaagc

SEQ ID NO: 138 - DNA, 34 bp, Artificial sequence
  Description of sequence Reverse primer R357H
catagatgcatgatagacctgaagttcaaaattg

SEQ ID NO: 139 - DNA, 26 bp, Artificial sequence
  Description of sequence Forward primer L909S
gagtgggtttcggctgagctggtggc

SEQ ID NO: 140 - DNA, 26 bp, Artificial sequence
  Description of sequence Reverse primer L909S
ctcagccgaaacccactcggtcacag

SEQ ID NO: 141 - DNA, 27 bp, Artificial sequence
  Description of sequence Forward primer A327D
cttcttggacaaagaaattggggatgc

SEQ ID NO: 142 - DNA, 34 bp, Artificial sequence
  Description of sequence Reverse primer A327D
caatttctttgtccaagaagttttgggacagatc

SEQ ID NO: 143 - DNA, 30 bp, Artificial sequence
  Description of sequence Forward primer K334E
gatgctgaatttctgcattttctccccagc

SEQ ID NO: 144 - DNA, 31 bp, Artificial sequence
  Description of sequence Reverse primer K334E
gcagaaattcagcatccccaatttctttggc

SEQ ID NO: 145 - DNA, 30 bp, Artificial sequence
  Description of sequence Forward primer P4L
tggtgtttctaatgtggacactgaagagac

SEQ ID NO: 146 - DNA, 31 bp, Artificial sequence
  Description of sequence Reverse primer P4L
gtccacattagaaacaccatgaattcccggc

SEQ ID NO: 147 - DNA, 28 bp, Artificial sequence
  Description of sequence Forward primer T1048M
caaggaaatggtctagccgcggtcatag

SEQ ID NO: 148 - DNA, 29 bp, Artificial sequence
  Description of sequence Reverse primer T1048M
cggctagaccatttccttgaacacctgac

SEQ ID NO: 149 - DNA, 38 bp, Artificial sequence
  Description of sequence Forward primer Y468H
cattatttcagacatgataagtatgcaaggagttgcag

SEQ ID NO: 150 - DNA, 41 bp, Artificial sequence
  Description of sequence Reverse primer Y468H
gcatacttatcatgtctgaaataatgtaattgttccaggac

SEQ ID NO: 151 - DNA, 37 bp, Artificial sequence
  Description of sequence Forward primer R422S
caatttaaaagcctgaaagtcatagatctttcagtga

SEQ ID NO: 152 - DNA, 34 bp, Artificial sequence
  Description of sequence Reverse primer R422S
gactttcaggcttttaaattgtttaaacatgctg

SEQ ID NO: 153 - DNA, 40 bp, Artificial sequence
  Description of sequence Forward primer V430L
gtcatagatctttcactgaataaaatatcaccttcaggag

SEQ ID NO: 154 - DNA, 39 bp, Artificial sequence
  Description of sequence Reverse primer V430L
gtgatattttattcagtgaaagatctatgactttcagtc

SEQ ID NO: 155 - DNA, 31 bp, Artificial sequence
  Description of sequence Forward primer H1038Y
cacagacaattatgtggcctatagtcaggtg

SEQ ID NO: 156 - DNA, 26 bp, Artificial sequence
  Description of sequence Reverse primer H1038Y
ccacataattgtctgtggccagggcg

SEQ ID NO: 157 - DNA, 30 bp, Artificial sequence
  Description of sequence Forward primer P457S
gttatgaatcccaggtcctggaacaattac

SEQ ID NO: 158 - DNA, 35 bp, Artificial sequence
  Description of sequence Reverse primer P457S
caggacctgggattcataactttctacagaagttc

SEQ ID NO: 159 - DNA, 40 bp, Artificial sequence
  Description of sequence Forward primer R473K
gataagtatgcaaagagttgcagattcaaaaacaaagagg

SEQ ID NO: 160 - DNA, 34 bp, Artificial sequence
  Description of sequence Reverse primer R473K
gaatctgcaactctttgcatacttatcatatctg

SEQ ID NO: 161 - DNA, 29 bp, Artificial sequence
  Description of sequence Forward primer T1035I
ctggccatagacaatcatgtggcctatag

SEQ ID NO: 162 - DNA, 28 bp, Artificial sequence
  Description of sequence Reverse primer T1035I
gattgtctatggccagggcgttctttag

SEQ ID NO: 163 - DNA, 30 bp, Artificial sequence
  Description of sequence Forward primer A448T
ctgctcaaataccagaacttctgtagaaag

SEQ ID NO: 164 - DNA, 32 bp, Artificial sequence
  Description of sequence Reverse primer A448T
gaagttctggtatttgagcagaagccaacttc

SEQ ID NO: 165 - DNA, 36 bp, Artificial sequence
  Description of sequence Forward primer R449K
gctcaaatgccaaaacttctgtagaaagttatgaac

SEQ ID NO: 166 - DNA, 31 bp, Artificial sequence
  Description of sequence Reverse primer R449K
cagaagttttggcatttgagcagaagccaac

SEQ ID NO: 167 - DNA, 28 bp, Artificial sequence
  Description of sequence Forward primer V442D
caagtgaagatggcttctgctcaaatgc

SEQ ID NO: 168 - DNA, 32 bp, Artificial sequence
  Description of sequence Reverse primer V442D
gcagaagccatcttcacttgaatctcctgaag

SEQ ID NO: 169 - DNA, 40 bp, Artificial sequence
  Description of sequence Forward primer G638S
atggagagaaagtgataacagatacttacaattattcaag

SEQ ID NO: 170 - DNA, 35 bp, Artificial sequence
  Description of sequence Reverse primer G638S
ctgttatcactttctctccataaaacatctaagtg

SEQ ID NO: 171 - DNA, 33 bp, Artificial sequence
  Description of sequence Forward primer T712N
caaccaactgaacactgtccctgagagattatc

SEQ ID NO: 172 - DNA, 30 bp, Artificial sequence
  Description of sequence Reverse primer T712N
gacagtgttcagttggttgtggctgaggtc

SEQ ID NO: 173 - DNA, 33 bp, Artificial sequence
  Description of sequence Forward primer T712S
caaccaactgagcactgtccctgagagattatc

SEQ ID NO: 174 - DNA, 30 bp, Artificial sequence
  Description of sequence Reverse primer T712S
gacagtgctcagttggttgtggctgaggtc

SEQ ID NO: 175 - DNA, 29 bp, Artificial sequence
  Description of sequence Forward primer S724R
ctgttccagacgcctcaagaatctgattc

SEQ ID NO: 176 - DNA, 31 bp, Artificial sequence
  Description of sequence Reverse primer S724R
cttgaggcgtctggaacagttggataatctc

SEQ ID NO: 177 - DNA, 24 bp, Artificial sequence
  Description of sequence Forward primer G145D
cgcaggacctcccgcctagcttac

SEQ ID NO: 178 - DNA, 27 bp, Artificial sequence
  Description of sequence Reverse primer G145D
cgggaggtcctgcggtatctctagtag

SEQ ID NO: 179 - DNA, 35 bp, Artificial sequence
  Description of sequence Forward primer D829N
gatctccctgaatctgtacacctgtgagttagatc

SEQ ID NO: 180 - DNA, 29 bp, Artificial sequence
  Description of sequence Reverse primer D829N
gtacagattcagggagatcacactttggc

SEQ ID NO: 181 - DNA, 26 bp, Artificial sequence
  Description of sequence Forward primer A819T
gccaggaacacacaagggccaaagtg

SEQ ID NO: 182 - DNA, 26 bp, Artificial sequence
  Description of sequence Reverse primer A819T
cttgtgtgttcctggccccacacaag

SEQ ID NO: 183 - DNA, 35 bp, Artificial sequence
  Description of sequence Forward primer D632E
cacttagaagttttatggagagaaggtgataacag

SEQ ID NO: 184 - DNA, 38 bp, Artificial sequence
  Description of sequence Reverse primer D632E
ctccataaaacttctaagtgatttcctctgaattccag

SEQ ID NO: 185 - DNA, 28 bp, Artificial sequence
  Description of sequence Forward primer L153I
cttacagcttatcagccttgaggccaac

SEQ ID NO: 186 - DNA, 29 bp, Artificial sequence
  Description of sequence Reverse primer L153I
ctcaaggctgataagctgtaagctaggcg

SEQ ID NO: 187 - DNA, 27 bp, Artificial sequence
  Description of sequence Forward primer L140I
cagctaatagagataccgcagggcctc

SEQ ID NO: 188 - DNA, 31 bp, Artificial sequence
  Description of sequence Reverse primer L140I
gcggtatctctattagctggtttccatccag

SEQ ID NO: 189 - DNA, 28 bp, Artificial sequence
  Description of sequence Forward primer T65M
gtattcccatgaacaccacgaacctcac

SEQ ID NO: 190 - DNA, 32 bp, Artificial sequence
  Description of sequence Reverse primer T65M
gtggtgttcatgggaatacctccaggaatttc

SEQ ID NO: 191 - DNA, 33 bp, Artificial sequence
  Description of sequence Forward primer L177F
caacatagaaatattctacctgggccaaaactg

SEQ ID NO: 192 - DNA, 32 bp, Artificial sequence
  Description of sequence Reverse primer L177F
caggtagaatatttctatgttggccagttctg

SEQ ID NO: 193 - DNA, 30 bp, Artificial sequence
  Description of sequence Forward primer Q138R
gatggaaaccggctactagagataccgcag

SEQ ID NO: 194 - DNA, 34 bp, Artificial sequence
  Description of sequence Reverse primer Q138R
gtatctctagtagccggtttccatccaggtaaag

SEQ ID NO: 195 - DNA, 35 bp, Artificial sequence
  Description of sequence Forward primer E175Q
gccaacatacaaatactctacctgggccaaaactg

SEQ ID NO: 196 - DNA, 37 bp, Artificial sequence
  Description of sequence Reverse primer E175Q
gtagagtatttgtatgttggccagttctgttagattc

SEQ ID NO: 197 - DNA, 35 bp, Artificial sequence
  Description of sequence Forward primer I195T
catattcaacagagaaagatgccttcctaaacttg

SEQ ID NO: 198 - DNA, 38 bp, Artificial sequence
  Description of sequence Reverse primer I195T
gaaggcatctttctctgttgaatatgaaacataacaag

SEQ ID NO: 199 - DNA, 39 bp, Artificial sequence
  Description of sequence Forward primer E170Q
gagaatctaacacaactggccaacatagaaatactctac

SEQ ID NO: 200 - DNA, 31 bp, Artificial sequence
  Description of sequence Reverse primer E170Q
gttggccagttgtgttagattctcttttctg

SEQ ID NO: 201 - DNA, 29 bp, Artificial sequence
  Description of sequence Forward primer L25P
caaactccctggggctagatggtttccta

SEQ ID NO: 202 - DNA, 33 bp, Artificial sequence
  Description of sequence Reverse primer L25P
ctagccccagggagtttggaaattaggattatg

SEQ ID NO: 203 - DNA, 29 bp, Artificial sequence
  Description of sequence Forward primer A809V
cttacctggtcacagatgtgacttgtgtg

SEQ ID NO: 204 - DNA, 33 bp, Artificial sequence
  Description of sequence Reverse primer A809V
cacatctgtgaccaggtaaggaatagtcacctc

SEQ ID NO: 205 - DNA, 35 bp, Artificial sequence
  Description of sequence Forward primer E543Q
caacctttagcacagctgagatatttggacttctc

SEQ ID NO: 206 - DNA, 28 bp, Artificial sequence
  Description of sequence Reverse primer E543Q
ctcagctgtgctaaaggttggaattcac

SEQ ID NO: 207 - DNA, 38 bp, Artificial sequence
  Description of sequence Forward primer E242del
gattgcaaaaatccaagatgattttaataacctcaacc

SEQ ID NO: 208 - DNA, 43 bp, Artificial sequence
  Description of sequence Reverse primer E242del
gaggttattaaaatcatcttggatttttgcaatcatgttgttg

SEQ ID NO: 209 - DNA, 34 bp, Artificial sequence
  Description of sequence Forward primer E242D
atcatcatcttggatttttgcaatcatgttgttg

SEQ ID NO: 210 - DNA, 34 bp, Artificial sequence; identical to SEQ ID NO: 209
  Description of sequence Reverse primer E242D

SEQ ID NO: 211 - DNA, 39 bp, Artificial sequence
  Description of sequence Reverse primer I847T
gagatacagatgtggaaagtgagaacagaatcaggttag

SEQ ID NO: 212 - DNA, 35 bp, Artificial sequence
  Description of sequence Forward primer I847T
cactttccacatctgtatctctctttctcatggtg

SEQ ID NO: 213 - DNA, 31 bp, Artificial sequence
  Description of sequence Forward primer Y546del
cagagctgagattggacttctccaacaaccg

SEQ ID NO: 214 - DNA, 36 bp, Artificial sequence
  Description of sequence Reverse primer Y546del
gttggagaagtccaatctcagctctgctaaaggttg

SEQ ID NO: 215 - DNA, 24 bp, Artificial sequence
  Description of sequence Forward primer L1029P
gcagtgtccaaagaacgccctggc

SEQ ID NO: 216 - DNA, 28 bp, Artificial sequence
  Description of sequence Reverse primer L1029P
gcgttctttggacactgccagaagtatg

SEQ ID NO: 217 - DNA, 33 bp, Artificial sequence
  Description of sequence Forward primer S618N
gaccatggagaatgagtctcttagaactctgga

SEQ ID NO: 218 - DNA, 27 bp, Artificial sequence
  Description of sequence Reverse primer S618N
agagactcattctccatggtcctgctg

SEQ ID NO: 219 - DNA, 33 bp, Artificial sequence
  Description of sequence Forward primer P31S
ctagatggttttctaaaactctgccctgtgatg

SEQ ID NO: 220 - DNA, 33 bp, Artificial sequence
  Description of sequence Reverse primer P31S
cagagttttagaaaaccatctagccccaaggag

SEQ ID NO: 221 - DNA, 33 bp, Artificial sequence
  Description of sequence Forward primer P307Q
catgtgccccaaagatggtttaagaacatcaac

SEQ ID NO: 222 - DNA, 28 bp, Artificial sequence
  Description of sequence Reverse primer P307Q
ccatctttggggcacatgctgaagagag

SEQ ID NO: 223 - DNA, 28 bp, Artificial sequence
  Description of sequence Forward primer H46R
caaagaaccgtgtgatcgtggactgcac

SEQ ID NO: 224 - DNA, 30 bp, Artificial sequence
  Description of sequence Reverse primer H46R
cgatcacacggttctttggaacatccagag

SEQ ID NO: 225 - DNA, 33 bp, Artificial sequence
  Description of sequence Forward primer D41H
gtcactctgcatgttccaaagaaccatgtgatc

SEQ ID NO: 226 - DNA, 28 bp, Artificial sequence
  Description of sequence Reverse primer D41H
gaacatgcagagtgacatcacagggcag

SEQ ID NO: 227 - DNA, 30 bp, Artificial sequence
  Description of sequence Forward primer K44R
gatgttccaaggaaccatgtgatcgtggac

SEQ ID NO: 228 - DNA, 34 bp, Artificial sequence
  Description of sequence Reverse primer K44R
cgatcacatggttccttggaacatccagagtgac

SEQ ID NO: 229 - DNA, 29 bp, Artificial sequence
  Description of sequence Forward primer M854I
ctttctcatagtgatgatgacagcaagtc

SEQ ID NO: 230 - DNA, 29 bp, Artificial sequence
  Description of sequence Reverse primer M854I
catcatcactatgagaaagagagatacag

SEQ ID NO: 231 - DNA, 31 bp, Artificial sequence
  Description of sequence Forward primer V38I
gccctgtgatatcactctggatgttccaaag

SEQ ID NO: 232 - DNA, 33 bp, Artificial sequence
  Description of sequence Reverse primer V38I
catccagagtgatatcacagggcagagttttag

SEQ ID NO: 233 - DNA, 30 bp, Artificial sequence
  Description of sequence Forward primer H298R
cgtctacgcagtaactctcttcagcatgtg

SEQ ID NO: 234 - DNA, 41 bp, Artificial sequence
  Description of sequence Reverse primer H298R
gaagagagttactgcgtagacgtaaaacttttaattctgtc

SEQ ID NO: 235 - DNA, 35 bp, Artificial sequence
  Description of sequence Forward primer N109Tfs*21
ctggggtcaaaaacaacatgtgcatcaagaggctg

SEQ ID NO: 236 - DNA, 35 bp, Artificial sequence
  Description of sequence Reverse primer N109Tfs*21
gcacatgttgtttttgaccccagtggaataggtac

SEQ ID NO: 237 - DNA, 32 bp, Artificial sequence
  Description of sequence Forward primer G331V
gccaaagaaattgtggatgctaaatttctgca

SEQ ID NO: 238 - DNA, 27 bp, Artificial sequence
  Description of sequence Reverse primer G331V
gcatccacaatttctttggccaagaag

SEQ ID NO: 239 - DNA, 28 bp, Artificial sequence
  Description of sequence Forward primer R296C
gttttatgtctacacagtaactctcttc

SEQ ID NO: 240 - DNA, 29 bp, Artificial sequence
  Description of sequence Reverse primer R296C
tgtgtagacataaaacttttaattctgtc

SEQ ID NO: 241 - DNA, 32 bp, Artificial sequence
  Description of sequence Forward primer G331W
gccaaagaaatttgggatgctaaatttctgca

SEQ ID NO: 242 - DNA, 27 bp, Artificial sequence
  Description of sequence Reverse primer G331W
gcatcccaaatttctttggccaagaag

SEQ ID NO: 243 - DNA, 34 bp, Artificial sequence; identical to SEQ ID NO: 48
  Description of sequence Forward primer A288V

SEQ ID NO: 244 - DNA, 37 bp, Artificial sequence; identical to SEQ ID NO: 47
  Description of sequence Reverse primer A288V

SEQ ID NO: 245 - DNA, 36 bp, Artificial sequence
  Description of sequence Forward primer D866N
ctatttctggaatgtgtggtatatttaccatttctg

SEQ ID NO: 246 - DNA, 29 bp, Artificial sequence
  Description of sequence Reverse primer D866N
cacacattccagaaatagaggtgacttgc

SEQ ID NO: 247 - DNA, 27 bp, Artificial sequence
  Description of sequence Forward primer L916M
gtggccaaaatggaagacccaagagag

SEQ ID NO: 248 - DNA, 27 bp, Artificial sequence
  Description of sequence Reverse primer L916M
gtcttccattttggccaccagctcagc

SEQ ID NO: 249 - DNA, 26 bp, Artificial sequence
  Description of sequence Forward primer V913M
ctgagctgatggccaaactggaagac

SEQ ID NO: 250 - DNA, 25 bp, Artificial sequence
  Description of sequence Reverse primer V913M
gtttggccatcagctcagccaaaac

SEQ ID NO: 251 - DNA, 40 bp, Artificial sequence
  Description of sequence Forward primer A962T
gacagacaagtatacaaagactgaaaattttaagatagca

SEQ ID NO: 252 - DNA, 33 bp, Artificial sequence
  Description of sequence Reverse primer A962T
tcagtctttgtatacttgtctgtcatcacaaac

SEQ ID NO: 253 - DNA, 29 bp, Artificial sequence
  Description of sequence Forward primer D959N
gtgatgacaaacaagtatgcaaagactga

SEQ ID NO: 254 - DNA, 30 bp, Artificial sequence
  Description of sequence Reverse primer D959N
gcatacttgtttgtcatcacaaacactgtc

SEQ ID NO: 255 - DNA, 32 bp, Artificial sequence
  Description of sequence Forward primer M957V
cagtgtttgtggtgacagacaagtatgcaaag

SEQ ID NO: 256 - DNA, 32 bp, Artificial sequence
  Description of sequence Reverse primer M957V
cttgtctgtcaccacaaacactgtctttttgc

SEQ ID NO: 257 - DNA, 33 bp, Artificial sequence
  Description of sequence Forward primer P272S
cttgtgcgtcgtgtaaaaataattctcccctac

SEQ ID NO: 258 - DNA, 25 bp, Artificial sequence
  Description of sequence Reverse primer P272S
tacacgacgcacaaggaaatggggc

SEQ ID NO: 259 - DNA, 26 bp, Artificial sequence
  Description of sequence Forward primer A1021V
ccgcaagttcacccatacttctggca

SEQ ID NO: 260 - DNA, 26 bp, Artificial sequence
  Description of sequence Reverse primer A1021V
atgggtgaacttgcgggtttgttggc

SEQ ID NO: 261 - DNA, 35 bp, Artificial sequence
  Description of sequence Forward primer F894C
gatgcttgtattgtgtatgacactaaagacccagc

SEQ ID NO: 262 - DNA, 36 bp, Artificial sequence
  Description of sequence Reverse primer F894C
catacacaatacaagcatcatagcaacagtctggtg

SEQ ID NO: 263 - DNA, 26 bp, Artificial sequence
  Description of sequence Forward primer L1001H
caagttccaccagctccggaaaaggc

SEQ ID NO: 264 - DNA, 28 bp, Artificial sequence
  Description of sequence Reverse primer L1001H
gagctggtggaacttggacttctgaaag

SEQ ID NO: 265 - DNA, 30 bp, Artificial sequence
  Description of sequence Forward primer K999R
gaagtccaggttcctccagctccggaaaag

SEQ ID NO: 266 - DNA, 31 bp, Artificial sequence
  Description of sequence Reverse primer K999R
ctggaggaacctggacttctgaaagggcttc

SEQ ID NO: 267 - DNA, 33 bp, Artificial sequence
  Description of sequence Forward primer L680I
ctaaagaatctatctttggccaaaaatgggctc

SEQ ID NO: 268 - DNA, 38 bp, Artificial sequence
  Description of sequence Reverse primer L680I
gccaaagatagattctttagatttggaggcataccatc

SEQ ID NO: 269 - DNA, 39 bp, Artificial sequence
  Description of sequence Forward primer F200L
gagaaagatgccctcctaaacttgacaaagttaaaagtg

SEQ ID NO: 270 - DNA, 37 bp, Artificial sequence
  Description of sequence Reverse primer F200L
gtcaagtttaggagggcatctttctctattgaatatg

SEQ ID NO: 271 - DNA, 69 bp, Artificial sequence
  Description of sequence Forward primer K764M
gatgatccaaatgaccagcttcccagaaaatggtcaagtttaggagggcatctttctctattgaatatg

SEQ ID NO: 272 - DNA, 68 bp, Artificial sequence
  Description of sequence Reverse primer K764M
gaagctggtcatttggatcatctggattttagtcaagtttaggagggcatctttctctattgaatatg

SEQ ID NO: 273 - DNA, 75 bp, Artificial sequence
  Description of sequence Forward primer E291Q
gatgcgctgacacaattaaaagttttacgtctacacaggtcaagtttaggagggcatctttctctattgaatatg

SEQ ID NO: 274 - DNA, 71 bp, Artificial sequence
  Description of sequence Reverse primer E291Q
cttttaattgtgtcagcgcatcaaaagcatttacgtcaagtttaggagggcatctttctctattgaatatg

SEQ ID NO: 275 - DNA, 76 bp, Artificial sequence
  Description of sequence Forward primer InFus. pCMV6-ECORI-F
gggcggccgggaattcatggtgtttccaatgtggacactgtcaagtttaggagggcatctttctctattgaatatg

SEQ ID NO: 276 - DNA, 79 bp, Artificial sequence
  Description of sequence Reverse primer InFus. pCMV6-SACII-R
gaaacagctatgaccgcggctagaccgtttccttgaacacctgtcaagtttaggagggcatctttctctattgaatatg

(19) United States
(12) Patent Application Publication
(10) Pub. No.: US 2023/0288414 A1
(43) Pub. Date: Sep. 14, 2023

(54) Type 1 IFN Assays and Methods of Diagnosis for Susceptibility to and Treatment of Viral Disease and Viral Vaccines, Including Covid -19

(71) Applicants: THE ROCKEFELLER UNIVERSITY, New York, NY (US); NSERM(Institut National de la Santé la Recherche Médicale), Paris (FR); Assistance Publique-Hôptiaux de Paris (APHP), Paris (FR); Université Paris Cité, Paris (FR); Fondation Imagine, Paris (FR)

(72) Inventor: Jean-Laurent Casanova, New York, NY (US)

(21) Appl. No.: 18/017,249

(22) PCT Filed: Jul. 22, 2021
(86) PCT No.: PCT/US2021/042741

§ 371 Date: Jan. 20, 2023

Related U.S. Application Data
(60) Provisional application No. 63/055,155, filed on Jul. 22, 2020.
(60) Provisional application No. 63/141,669, filed on Jan. 26, 2021.

(51) Int. Cl. G01N 33/564

(52) U.S. Cl. CPC G01N 33/564; G01N 2800/52; G01N 2800/24; G01N 2333/555; G01N 2800/50

(57) ABSTRACT

The present invention provides methods, assays and kits for assessment of patients positive for SARS-CoV-2 infection and methods of diagnosis and treatment of COVID-19 disease and for assessment and evaluation of individuals prior to vaccination with live attenuated virus vaccines, particularly including yellow fever vaccines and COVID-19 vaccines, to assess risk for vaccine-associated disease and adverse events, and for evaluation, treatment and management of patients who develop vaccine-associated disease. The invention provides methods and assays for identification and characterization of inborn errors of type I interferon immunity and also auto-antibodies against Type I IFNs that are associated with severe COVID-19 disease or that are correlated and linked with vaccine-associated disease. The invention further provides methods of diagnosing and determining altered response to or susceptibility to SARS-CoV-2 infection or to live attenuated virus vaccines and for applicable and suitable treatment of COVID-19 disease or vaccine-associated disease.

### STATEMENT OF GOVERNMENT RIGHTS

This invention was made with government support under numbers R01AI088364, UL1TR001866, UM1HG006504 and U24HG008956 awarded by the National Institutes of Health. The government has certain rights in the invention.

### FIELD OF THE INVENTION

The present invention relates generally to the assessment of patients positive for SARS-CoV-2 infection and to methods of diagnosis and treatment of COVID-19 disease as well as to the evaluation of individuals prior to vaccination with live attenuated virus vaccines, particularly including yellow fever vaccines, to assess risk for vaccine-associated disease and adverse events, and for evaluation, treatment and management of patients who develop vaccine-associated disease.

### BACKGROUND OF THE INVENTION

Coronaviruses are a family of viruses that can cause illnesses such as the common cold, severe acute respiratory syndrome (SARS) and Middle East respiratory syndrome (MERS). Coronaviruses are positive sense, single-strand enveloped RNA virus belonging to the family Coronaviridae. In late 2019, a new coronavirus, severe acute respiratory syndrome coronavirus 2 (SARS-CoV-2), was identified as the cause of a COVID-19 disease outbreak that originated in China. In March 2020, the World Health Organization (WHO) declared the COVID-19 outbreak a pandemic.

SARS-CoV-2 is responsible for the COVID-19 pandemic, which has already claimed at least 600,000 deaths (1). Most life-threatening cases begin with a pneumonia, which progresses to acute respiratory distress syndrome (ARDS) and the failure of other organs (2-4). SARS-CoV-2 infection has been diagnosed by pharyngeal PCR in over 14 million people, most of whom had mild, self-healing disease (1). The global number of infected individuals is at least 10 times higher, as suggested by recent serological and virological studies (5). The clinical manifestations of infection with SARS-CoV2 thus range from silent infection to lethal disease (6-8). Globally, infection-fatality rate ranges from 0.1% to 0.9% (9). Two epidemiological factors increase the risk of severity, whether defined as hospitalization or death. The most striking is age; risk increases sharply, decade by decade, after the age of 50 years (6, 10). Various underlying medical conditions constitute a second risk factor (11).

Live attenuated vaccines (LAVs) contain versions of the relevant virus that have been altered and weakened so they do not cause serious disease in people with healthy immune systems. They stimulate protective immune responses when they replicate in the host. As of late year 2018, there were 11 live attenuated viral (LAV) vaccines in use worldwide. All have been shown or suspected to cause life threatening disease in rare individuals. Common LAVs include those for measles, mumps, rubella (MMR combined vaccine), small pox, and yellow fever. Severe disease by LAV vaccines of live oral polio, oral rotavirus and measles, mumps rubella (MMR) has been explained by inborn errors of immunity, in at least some patients. Live (oral) poliovirus. The measles vaccine strain in the measles, mumps, and rubella (MMR) vaccine can cause disseminated infections in patients with inborn errors of IFNAR2 (Duncan C J A et al., 2015), STAT1 (Burns C et al., 2016 J. Allergy Clin. Immunol. Pract. 4:777-779), or STAT2 (Hambleton et al., 2013; Moens L et al., 2017 J. Allergy Clin. Immunol. 139:1995-1997.e9), which control cellular responses to various IFNs (type I only in the case of IFNAR2, type I and type III IFNs for STAT2, and all three types for STAT1). Vaccine-strain mumps infections are extremely rare, with only one life-threatening case reported in a patient with RAG1 deficiency and a lack of T and B cell adaptive immunity (Morfopoulou S et al., 2017 Acta Neuropathol. 133:139-147). Patients with inborn errors of IRF7 (Ciancanelli M J et al., 2015 Science. 348:448-453), which controls the amplification of type I and III IFNs, or IRF9 (Hernandez et al., 2018), which controls the cellular responses to these IFNs, have been reported to suffer from an ill-defined adverse reaction to MMR vaccination. Most cases of severe MMR vaccine disease remain unexplained.

Yellow fever virus is an RNA virus that belongs to the genus Flavivirus and is related to West Nile, St. Louis encephalitis, and Japanese encephalitis viruses. Yellow fever virus is is found in tropical and subtropical areas of Africa and South America and is transmitted primarily through infected Aedes or Haemagogus species mosquitoes. There is no medicine to treat or cure infection. Prevention of virus infection is promoted via use of insect repellent, long-sleeved shirts and long pants, and vaccination. Yellow fever vaccine is recommended for people aged 9 months or older and who are traveling to or living in areas at risk for yellow fever virus in Africa and South America and may be required for entry into certain countries. Despite the development of the Yellow fever virus vaccine in the 1930s, Yellow fever virus remains a global health threat partially due to deficient rates of vaccination.

The 17D live-attenuated vaccine against yellow fever virus (YFV) was approved for use in humans by the World Health Organization in 1945. It has since been used to vaccinate more than 600 million people worldwide, with very high rates of seroconversion following the administration of a single dose, providing long-term protection (Monath, 2005; Monath et al., 2005). About half the vaccine recipients develop transient low-level viremia detectable four to six days after inoculation, a timing similar to that for viremia after wild-type YFV infection. All live attenuated YFV vaccines in current use are derivatives of the 17D strain and produced by amplification in embryonated chicken eggs. Although initially considered to be the world's safest live virus vaccine, rare cases of life-threatening disease following vaccination with YFV-17D were subsequently detected, from 2001 onward (Chan et al., 2001; Martin et al., 2001; Seligman, 2014; Vasconcelos et al., 2001). Systemic disease with clinical manifestations of organ dysfunction is often reported as yellow fever vaccine associated viscerotropic disease (YEL-AVD) (Seligman, 2014), and cases with neurological manifestations are referred to as yellow fever vaccine-associated neurological disease (YELAND). The prevalences of these conditions are about 0.3 and 0.8 per 100,000 vaccinees, respectively (Lindsey et al., 2016). However, prevalence estimates vary, ranging, for YELAVD, from 0-0.01 per 100,000 vaccinees in Africa, to 0.02-0.31 per 100,000 vaccinees in Brazil, and 0.35 per 100,000 vaccinees in USA, which is considered to be the most accurate estimate. The incidence of YEL-AND is estimated at 0.39 per 105 administered vaccine doses (range 0.02-1.5) (Lecomte et al., 2020). Mortality rates vary depending on age, but approximatively two thirds of individuals die (Seligman, 2014). The rate of severe adverse events seems to increase with age, particularly after the age of 55 years, and is higher in men (Lindsey et al., 2016; Seligman, 2014). Women in their prime child-bearing years and patients with thymoma are also at risk (Seligman, 2014). Severe adverse reactions have also occurred in association with various autoimmune diseases, including systemic lupus erythematosus (SLE), Addison's disease, pernicious anemia, and myasthenia gravis, suggesting an immunological mechanism (de Menezes Martins et al., 2014; Seligman, 2014; Seligman and Casanova, 2016).

Although still considered a safe and effective vaccine, the uncertainty regarding the causes of YEL-AVD have encouraged attempts to develop an inactivated alternative vaccine (Monath T P et al., 2011 N. Engl. J. Med. 364:1326-1333; Monath T P and Vasconcelos P F C, 2015 J. Clin. Virol. 64: 160-173; Pereira R C et al., 2015 Vaccine. 33:4261-4268). The difficulties encountered while attempting to develop safer vaccines related to YFV-17D for the past decade highlight that a better understanding of host defenses against the virus is necessary.

There are a number of COVID-19 vaccines in development which are live attenuated vaccines. For example, Sanchez-Felipe L et al describe the development of a candidate vaccine (YF-S0) for severe acute respiratory syndrome coronavirus 2 (SARS-CoV-2) that uses live-attenuated yellow fever 17D (YF17D) vaccine as a vector to express a noncleavable prefusion form of the SARS-CoV-2 spike antigen (Sanchez-Felipe L et al Nature. 2021 February; 590(7845):320-325. doi: 10.1038/s41586-020-3035-9). COVI-VAC is a live attenuated virus being developed by Codagenix (Wang Y et al (2021) PNAS 118(29); e2102775118). Meissa is developing an RSV-based LAV using a weakened RSV virus harboring coronavirus spike protein. Another Live attenuated SARS-CoV-2 vaccine candidate has been decribed by Okamura (Okamura, S. et al. (2021) doi.org/10.1101/2021.02.15.430863).

There is immense inter-individual clinical variability upon infection with SARS-CoV-2, ranging from silent infection to lethal COVID-19. However, there remains a lack of understanding and available information to explain the clinical variability. Furthermore, there are no accepted or approved treatments available for COVID-19 and no vaccine available against the SARS-CoV-2 virus. There is need to for methods to identify those individuals at greater risk for severe disease in order that they can be more aggressively managed and clinically treated. There is a need for identification of clinical, physiological, genetic aspects in patients that will characterize their risk of severe disease and identify their susceptibilities and for the suitable and applicable treatment for those patients. There is need to for methods to identify those individuals at risk for Yellow fever vaccine associated disease including YEL-AVD and YEL-AND. There remains a need for improved disease management among SARS-CoV-2 positive individuals and COVID-19 patients. The present invention addresses such unmet needs in the field, including as to virus infections and particularly with regard to SARS-CoV-2 virus and COVID-19 disease. The present invention addresses such unmet needs in the field, including as to yellow fever vaccine-associated disease and yellow fever virus infections

The citation of references herein shall not be construed as an admission that such is prior art to the present invention.

### SUMMARY OF THE INVENTION

In a general aspect, the present invention relates to previously unidentified and unknown genetic aspects of the interferon gene pathway that are correlated with and render patients susceptible to severe COVID-19 disease. In a general aspect, the present invention relates to previously unidentified and unknown genetic aspects of the interferon gene pathway that are correlated with and render patients susceptible to vaccine-associated disease, particularly live-attenuated vaccine related disease. In a general aspect, the present invention relates to previously unidentified and unknown genetic aspects of the interferon gene pathway that are correlated with and render patients susceptible to vaccine-associated disease, particularly yellow fever vaccine related disease, including YEL-AVD and YEL-AND. In a general aspect, the present invention relates to previously unidentified and unknown genetic aspects of the interferon gene pathway that are correlated with and render patients susceptible to vaccine-associated disease, particularly coronavirus particularly Sars-Cov2 vaccine related disease, including disease caused by, resulting from, associated with or related to live attenuated coronavirus vaccine(s). In a further general aspect, the invention relates to auto-antibodies directed against and specific for type I interferon proteins, the presence of which are correlated with and render patients susceptible to severe COVID-19 disease and/or to vaccine-associated disease.

In accordance with the invention and as described herein, at least or about 10% of patients with life threatening COVID-19 pneumonia have neutralizing auto-antibodies (auto-Abs) against type I IFNs. The neutralizing auto-Abs against type I IFNs are X-linked and are particularly prevalent and biased in males, or in females with an X-linked condition such that they express the same or preferentially express a single X chromosome. In an embodiment, females having X-linked incontinentia pigmenti (IP) may have neutralizing auto-Abs against type I IFNs. The invention provides assays, kits and methods for assessment of patients positive for SARS-CoV-2 infection and to methods of diagnosis and treatment of COVID-19 disease. The invention provides assays and methods for identification and characterization of auto-antibodies against Type I IFNs that are associated with severe COVID-19 disease. The invention further provides methods of diagnosing and determining altered response to or susceptibility to SARS-CoV-2 infection and for applicable and suitable treatment of COVID-19 disease.

In another general aspect, the invention relates to auto-antibodies directed against and specific for type I interferon proteins, the presence of which are correlated with and render patients susceptible to vaccine-associated disease, particularly yellow fever vaccine-associated disease, including YEL-AVD and YEL-AND. It has been recognized and confirmed that patients suffered adverse reactions to yellow fever virus live attenuated vaccine and yellow fever virus YFV-17D vaccination because of preexisting neutralizing antibodies against type I IFNs. The autoantibodies particularly targeted IFN-α2 and IFN-ω. The autoantibodies targeted most of the individual subtypes of type I IFNs. Autoantibodies targeting IFN-β were also identoified in certain patients.

In another general aspect, the invention relates to and identifies inborn errors of type I IFN immunity which are associated with and identifiable in patients with severe COVID-19. In particular, variations in type I IFN-related autosomal genes have been identified in patients with life-threatening COVID-19 pneumonia. In accordance with the invention, loss of function (LOF) mutations in one or more of IFNAR1, IRF7, IFIH1, TLR3, TBK1, IRF3, TICAM1 and UNC93B1 genes can be identified in patients with severe COVID-19 and are associated with and causal to aspects of severe COVID-19 disease. In accordance with the invention, mutations, which may include loss of function (LOF) mutations, in the TLR7 gene can be identified in patients with severe COVID-19 and are associated with and causal to aspects of severe COVID-19 disease. In particular, the TLR7 gene mutations are X-linked recessive mutations and can be identified in male patients with severe COVID-19 and are associated with and causal to aspects of severe COVID-19 disease. In an embodiment, mutations in IFNAR1, IRF7, IFIH1, TLR3, TBK1, IRF3, TICAM1, UNC93B1 and IFNAR2 can be identified in patients with severe COVID-19 and are associated with and causal to aspects of severe COVID-19 disease. In another embodiment, mutations in IFNAR1, IRF7, IFIH1, TLR3, TBK1, IRF3, TICAM1, UNC93B1, IFNAR2, STAT1, STAT2 or TRAF3 can be identified in patients with severe COVID-19 and are associated with and causal to aspects of severe COVID-19 disease. In another embodiment, mutations in IFNAR1, IRF7, IFIH1, TLR3, TBK1, IRF3, TICAM1, UNC93B1, IFNAR2, STAT1, STAT2, TRAF3 or IRF9 can be identified in patients with severe COVID-19 and are associated with and causal to aspects of severe COVID-19 disease. In an embodiment, mutations in TLR7 can be identified in patients, partiocularly in male patients, including in male patients below the age of 70 years, with severe COVID-19 and are associated with and causal to aspects of severe COVID-19 disease. Identification of any one or more of a LOF mutation or a mutation resulting in significantly reduced or inactive type-I IFN selected from one or more of IFNAR1, IRF7, IFIH1, TLR3, TBK1, IRF3, TICAM1, UNC93B1, IFNAR2, STAT1, STAT2, TRAF3 or IRF9, one or more of IFNAR1, IRF7, IFIH1, TLR3, TBK1, IRF3, TICAM1, UNC93B1, IFNAR2, STAT1, STAT2 or TRAF3 one or more of IFNAR1, IRF7, IFIH1, TLR3, TBK1, IRF3, TICAM1, UNC93B1 and IFNAR2, or one or more of IFNAR1, IRF7, IFIH1, TLR3, TBK1, IRF3, TICAM1 and UNC93B1 in an individual positive for SARS-CoV-2 infection provides critical information that the individual is altered in type I IFN response and most vulnerable to severe disease. Identification of any one or more of a LOF mutation or a mutation in the TLR7 gene resulting in significantly reduced or inactive TLR7 protein in an individual, particularly a male individual, positive for SARS-CoV-2 infection provides critical information that the individual is altered in type I IFN response and in protective type I IFN immunity against SARS-CoV-2 and most vulnerable to severe disease, particularly to severe pulmonary and lung disease. Such an individual(s) or patient(s) must be managed and treated differently and with particular and specific care so as to avoid severe disease and pneumonia.

The presence of inborn errors of type I interferon immunity or auto-antibodies against Type I IFNs dictates and defines aspects and approaches to therapy for COVID-19 disease. Also, the presence of inborn errors of type I interferon immunity or auto-antibodies against Type I IFNs can dictate and define aspects and approaches to therapy for vaccine-associated disease, particularly live-attenuated vaccine-associated disease, such as yellow fever vaccine-associated disease, such as coronavirus vaccine-associated disease, such as COVID-19 vaccine-associated disease, such as live-attenuated COVID-19 vaccine-associated disease. In certain embodiments, the presence of loss of function recessive or dominant, homozygous or heretozygous mutations in type I IFN genes results in an altered and/or nonfunctional or ineffective immune or IFN-mediated response to virus infection, thereby resulting in pathological and severe COVID-19 disease with SARS-CoV-2 infection. In certain embodiments, the presence of loss of function recessive or dominant, homozygous or heretozygous mutations in the X-linked TLR7 gene results in an altered and/or nonfunctional or ineffective immune or IFN-mediated response to virus infection, thereby resulting in pathological and severe COVID-19 disease with SARS-CoV-2 infection, particularly in males, including in young males, including males lass than 70 years old. In certain embodiments, the presence of auto-antibodies directed against Type I IFNs results in an altered and/or nonfunctional or ineffective immune or IFN-mediated response to virus infection and live attenuated virus vaccination, thereby resulting in pathological and severe yellow fever vaccine-associated disease or in pathological and severe coronavirus vaccine-associated disease, such as live-attenuated COVID-19 vaccine-associated disease. Identification of the type I IFN gene mutation present in a patient permits and enables the specific and targeted therapy such as administering the IFN protein which production or function is lost to the patient.

In certain embodiments, the presence of auto antibodies directed against Type I IFNs results in an altered and/or nonfunctional or ineffective immune or IFN-mediated response to virus infection, thereby resulting in pathological and severe COVID-19 disease with SARS-CoV-2 infection. Treatment of individuals having auto-antibodies directed against one or more Type I IFN with a type I IFN protein therapy will not result in improvement or therapy against the SARS-CoV-2 infection and could result in a significant or severe immune reaction against the IFN protein administered and/or could raise more significant neutralizing antibodie against the protein.

In certain embodiments, the presence of auto antibodies directed against Type I IFNs results in an altered and/or nonfunctional or ineffective immune or IFN-mediated response to virus infection and live attenuated virus vaccination, thereby resulting in pathological and severe vaccine-associated disease, such as pathological and severe yellow fever vaccine-associated disease, or such as pathological and severe COVID-19 vaccine-associated disease. Treatment of individuals having auto-antibodies directed against one or more Type I IFN with a type I IFN protein therapy will not result in improvement or therapy against the vaccine-associated disease, such as yellow fever vaccine-associated disease or COVID-19 vaccine-associated disease and could result in a significant or severe immune reaction against the IFN protein administered and/or could raise more significant neutralizing antibodies against the protein. In embodiments, administration of a type I IFN protein to which or against which the patient or individual does not have existing auto-antibodies is contemplated to facilitate treatment or remediation of vaccine-associated disease, such as yellow fever vaccine- or COVID-19 vaccine-associated disease.

The invention provides a method for evaluating the presence of anti-type I IFN specific auto-antibodies (auto-Abs) in a patient or individual positive for or at risk for SARS-CoV-2 infection or having COVID-19 disease and thereby determining treatment and treating the patient or individual comprising:
(a) isolating a blood or serum sample from said patient or individual;
(b) evaluating the blood or serum sample for auto-antibodies specific for one or more type I IFN selected from:
   (i) IFN-α2 and IFN-ω;
   (ii) IFN-α2, IFN-ω and IFN-β;
   (iii) IFN-α2, IFN-ω, IFN-β and IFN-ε;
   (iv) IFN-α2, IFN-ω, IFN-β, IFN-ε and IFN-κ;
   (v) IFN-α2, IFN-ω, IFN-β, IFN-ε, IFN-α1, IFN-α2, IFN-α6, IFN-α13, IFN-α14 and IFN-α16; and
   (vi) IFN-α2, IFN-ω, IFN-α1, IFN-α2, IFN-α6, IFN-α13, IFN-α14 and IFN-α16;
(c) wherein a patient or individual is treated to remove or deplete the auto-Abs and/or is administered a type I IFN against which they do not have auto-Abs and/or is administered an immune-modulatory agent that increases or facilitates type I IFN-mediated response.

The invention provides a method for evaluating the presence of anti-type I IFN specific auto-antibodies (auto-Abs) in a patient or individual prior to vaccination with live attenuated vaccine (LAV) or having vaccine-associated disease and thereby determining whether the patient or individual can safely receive LAV and/or treating the patient or individual comprising:
(a) isolating a blood or serum sample from said patient or individual;
(b) evaluating the blood or serum sample for auto-antibodies specific for one or more type I IFN selected from:
   (i) IFN-α2 and IFN-ω;
   (ii) IFN-α2, IFN-ω and IFN-β;
   (iii) IFN-α2, IFN-ω, IFN-β and IFN-α1/13;
   (iv) IFN-α2, IFN-ω, IFN-β, IFN-α1/13 and IFN-α14;
   (v) IFN-α2, IFN-ω, IFN-β, IFN-α1/13, IFN-α14 and IFN-α7; and
   (vi) IFN-α2, IFN-ω, IFN-α1/13, IFN-α14, IFN-α7;
(c) wherein a patient or individual having one or more auto-antibody is not administered the vaccine or wherein a patient or individual having vaccine-associated disease is treated to remove or deplete the auto-Abs and/or is administered a type I IFN against which they do not have auto-Abs and/or is administered an immune-modulatory agent that increases or facilitates type I IFN-mediated response.

The invention provides a method for evaluating the presence of anti-type I IFN specific auto-antibodies (auto-Abs) in a patient or individual prior to vaccination with live attenuated vaccine (LAV) against yellow fever virus or against COVID-19 virus or having yellow fever vaccine-associated disease or having COVID-19 vaccine-associated disease and thereby determining whether the patient or individual can safely receive YFV LAV or COVID-19 LAV and/or treating the patient or individual comprising:
(a) isolating a blood or serum sample from said patient or individual;
(b) evaluating the blood or serum sample for auto-antibodies specific for one or more type I IFN selected from:
   (i) IFN-α2 and IFN-ω;
   (ii) IFN-α2, IFN-ω and IFN-β;
   (iii) IFN-α2, IFN-ω, IFN-β and IFN-α1/13;
   (iv) IFN-α2, IFN-ω, IFN-β, IFN-α1/13 and IFN-α14;
   (v) IFN-α2, IFN-ω, IFN-β, IFN-α1/13, IFN-α14 and IFN-α7; and
   (vi) IFN-α2, IFN-ω, IFN-α1/13, IFN-α14, IFN-α7;
(c) wherein a patient or individual having one or more auto-antibody is not administered the vaccine or wherein a patient or individual having yellow fever vaccine- or COVID-19 vaccine-associated disease is treated to remove or deplete the auto-Abs and/or is administered a type I IFN against which they do not have auto-Abs and/or is administered an immune-modulatory agent that increases or facilitates type I IFN-mediated response.

The invention provides a method for evaluating the presence of anti-type I IFN specific auto-antibodies (auto-Abs) in a patient or individual prior to vaccination with live attenuated vaccine (LAV), such as against yellow fever virus or against COVID-19 and thereby determining whether the patient or individual can safely receive YFV LAV or COVID-19 LAV comprising:
(a) isolating a blood or serum sample from said patient or individual;
(b) evaluating the blood or serum sample for auto-antibodies specific for one or more type I IFN selected from:
   (i) IFN-α2 and IFN-ω;
   (ii) IFN-α2, IFN-ω and IFN-β;
   (iii) IFN-α2, IFN-ω, IFN-β and IFN-α1/13;
   (iv) IFN-α2, IFN-ω, IFN-β, IFN-α1/13 and IFN-α14;
   (v) IFN-α2, IFN-ω, IFN-β, IFN-α1/13, IFN-α14 and IFN-α7; and
   (vi) IFN-α2, IFN-ω, IFN-α1/13, IFN-α14, IFN-α7;
(c) wherein a patient or individual having one or more auto-antibody is not administered the vaccine.

The invention provides a method for evaluating the presence of anti-type I IFN specific auto-antibodies (auto-Abs) in a patient or individual prior to vaccination with live attenuated vaccine (LAV), particularly against yellow fever virus or against COVID-19 or SARSCoV-2 and thereby determining whether the patient or individual can safely receive YFV LAV or COVID-19 LAV comprising:
(a) isolating a blood or serum sample from said patient or individual;
(b) evaluating the blood or serum sample for auto-antibodies specific for one or more type I IFN selected from:
   (i) IFN-α2 and IFN-ω;
   (ii) IFN-α2, IFN-ω and IFN-β;
   (iii) IFN-α2, IFN-ω, IFN-β and IFN-α1/13;
   (iv) IFN-α2, IFN-ω, IFN-β, IFN-α1/13 and IFN-α14;
   (v) IFN-α2, IFN-ω, IFN-β, IFN-α1/13, IFN-α14 and IFN-α7; and
   (vi) IFN-α2, IFN-ω, IFN-α1/13, IFN-α14 and IFN-α7;
(c) wherein a patient or individual having one or more auto-antibody is not administered the vaccine or is treated to remove or deplete the auto-Abs and/or is administered a type I IFN against which they do not have auto-Abs and/or is administered an immune-modulatory agent that increases or facilitates type I IFN-mediated response.

In an embodiment of the method, the blood or serum sample is additionally or alternatively evaluated for auto-antibodies specific for one or more type I IFN selected from IFN-α4, IFN-α5, IFN-α6, IFN-α8, IFN-α10, IFN-α16, IFN-α17 and IFN-α21. In an embodiment of the method, the blood or serum sample is additionally or alternatively evaluated for auto-antibodies specific for one or more type I IFN selected from IFN-κ and IFN-ε.

In an embodiment of the method, the blood or serum sample is evaluated for neutralizing antibodies.

In an embodiment of the method, plasmapheresis is conducted on the patient or individual to deplete the antibodies, or wherein B cells, such as auto-reactive B cells, and/or plasmacytes or plasmablasts are depleted in the patient or individual.

In an embodiment of the method, a patient or individual having auto-Abs against one or more of IFN-α2, IFN-ω, IFN-ε, IFN-α1, IFN-α2, IFN-α6, IFN-α13, IFN-α14 or IFN-α16 and not having auto-Abs against IFN-β is treated by administering IFN-β.

In an embodiment of the method, a patient or individual having auto-Abs against one or more Type I IFN selected from IFN-ω, IFN-ε, IFN-β, IFN-κ, and not having auto-Abs against IFN-α2 is treated by administering IFN-α2.

In an embodiment of the method, a patient or individual having auto-Abs against one or more Type I IFN is treated by administering an IFN subtype which is not neutralized by the patient's or individual's auto-Abs.

In an embodiment of the method, a patient or individual having auto-Abs against one or more of Type I IFN is treated by administering an IFN-α subtype which is not neutralized by the patient's or individual's auto-Abs.

In an embodiment of the method, a patient or individual having auto-Abs against one or more of IFN-α2 or IFN-ω and not having auto-Abs against IFN-β is treated by administering IFN-β. In an embodiment of the method, a patient or individual having auto-Abs against one or more of IFN-α2, IFN-ω, IFN-α1/13, IFN-α14 or IFN-α7 and not having auto-Abs against IFN-β is treated by administering IFN-β. In an embodiment of the method, a patient or individual having auto-Abs against one or more of IFN-α2, IFN-ω, IFN-α1/13, IFN-α14, IFN-α7, IFN-α4, IFN-α5, IFN-α6, IFN-α8, IFN-α10, IFN-α16, IFN-α17 or IFN-α21 and not having auto-Abs against IFN-β is treated by administering IFN-β.

The invention further provides an assay for evaluating the presence of auto-antibodies directed against one or more Type I IFN in a patient or individual positive for or at risk for SARS-CoV-2 infection or having COVID-19 disease comprising:
(a) contacting a sample of blood or serum from the patient or individual with one or more recombinant type I IFN protein selected from IFN-α2, IFN-ω, IFN-β, IFN-ε and IFN-κ, wherein each IFN protein is labeled with a distinct detectable tag or marker to form an antibody-protein complex;
(b) contacting any antibody-protein complex of (a) with one or more labeled anti-human immune globulin molecule that will bind and label auto-antibody bound to any one or more IFN protein;
(c) specifically and selectively detecting each type I IFN protein bound by specific auto-Ab thereto.

The invention further provides an assay for evaluating the presence of auto-antibodies directed against one or more Type I IFN in a patient or individual prior to vaccination with live attenuated vaccine (LAV), such as LAV against yellow fever virus or against COVID-19 or having yellow fever vaccine- or COVID-19 vaccine-associated disease comprising:
(a) contacting a sample of blood or serum from the patient or individual with one or more recombinant type I IFN protein selected from IFN-α2, IFN-ω, IFN-β, IFN-ε and IFN-κ, wherein each IFN protein is labeled with a distinct detectable tag or marker to form an antibody-protein complex;
(b) contacting any antibody-protein complex of (a) with one or more labeled anti-human immune globulin molecule that will bind and label auto-antibody bound to any one or more IFN protein;
(c) specifically and selectively detecting each type I IFN protein bound by specific auto-Ab thereto.

The invention further provides an assay for evaluating the presence of auto-antibodies directed against one or more Type I IFN in a patient or individual prior to vaccination with live attenuated vaccine (LAV) against yellow fever virus or COVID-19 virus or having vaccine-associated disease, yellow fever vaccine-associated disease or COVID-19 vaccine-associated disease comprising:
(a) contacting a sample of blood or serum from the patient or individual with one or more recombinant type I IFN protein selected from IFN-α2, IFN-ω and IFN-β, wherein each IFN protein is labeled with a distinct detectable tag or marker to form an antibody-protein complex;
(b) contacting any antibody-protein complex of (a) with one or more labeled anti-human immune globulin molecule that will bind and label auto-antibody bound to any one or more IFN protein;
(c) specifically and selectively detecting each type I IFN protein bound by specific auto-Ab thereto.

In an embodiment of the assay, one or more recombinant type I IFN protein is labeled with a fluorescent marker.

In an embodiment of the assay, one or more recombinant type I IFN protein is covalently coupled to a magnetic bead with a fluorescent marker.

In an embodiment of the assay, each of the one or more recombinant type I IFN proteins is covalently coupled to a magnetic bead with a distinct and differential fluorescent marker.

In an embodiment of the assay, the one or more labeled anti-human immune globulin molecule that will bind and label bound auto-antibody is a labeled non-human animal derived anti-human IgG.

In an embodiment of the assay, the one or more recombinant type I IFN protein is selected from IFN-α2, IFN-ω and IFN-β.

In an embodiment of the assay, the one or more recombinant type I IFN protein is selected from IFN-α2 and IFN-ω.

In further embodiments of the assay, the presence of neutralizing auto-antibodies is determined.

The invention provides a kit for for evaluating the presence of auto-antibodies directed against one or more Type I IFN in a patient or individual at risk of, suspected of or determined to have coronavirus comprising:
(a) one or more recombinant type I IFN protein selected from IFN-α2, IFN-ω, IFN-β, IFN-ε and IFN-κ, wherein each IFN protein is labeled with a distinct detectable tag or marker;
(b) one or more labeled anti-human immune globulin molecule that will bind and label auto-antibody bound to any one or more IFN protein;
(c) a means for specific and selective detection of each type I IFN protein bound by specific auto-Ab thereto.

The invention provides a kit for evaluating the presence of auto-antibodies directed against one or more Type I IFN in a patient or individual prior to vaccination with live attenuated vaccine (LAV), such as LAV against yellow fever virus or COVID-19 or SARsCov-2 virus or having yellow fever vaccine- or COVID-19-associated disease comprising:
(a) one or more recombinant type I IFN protein selected from IFN-α2, IFN-ω and IFN-β wherein each IFN protein is labeled with a distinct detectable tag or marker;
(b) one or more labeled anti-human immune globulin molecule that will bind and label auto-antibody bound to any one or more IFN protein;
(c) a means for specific and selective detection of each type I IFN protein bound by specific auto-Ab thereto.

In an embodiment of the kit, one or more recombinant type I IFN protein is labeled with a fluorescent marker.

In an embodiment of the kit, one or more recombinant type I IFN protein is covalently coupled to a magnetic bead with a fluorescent marker.

In an embodiment of the kit, each of the one or more recombinant type I IFN proteins is covalently coupled to a magnetic bead with a distinct and differential fluorescent marker.

In an embodiment of the kit, the one or more labeled anti-human immune globulin molecule that will bind and label bound auto-antibody is a labeled non-human animal derived anti-human IgG.

In an embodiment of the kit, the one or more recombinant type I IFN protein is selected from IFN-α2, IFN-ω and IFN-β.

In an embodiment of the kit, wherein the one or more recombinant type I IFN protein is selected from IFN-α2 and IFN-ω.

The invention further provides a method for evaluating a patient or individual positive for or at risk for SARS-CoV-2 infection or having COVID-19 disease for the presence of a loss-of-function (LOF) variation or mutation in a type I IFN pathway gene and thereby determining treatment and treating the patient or individual comprising:
(a) isolating a blood or serum sample from said patient or individual;
(b) evaluating the blood or serum sample for a LOF variation or mutation in one or more type I IFN pathway gene selected from:
   (i) IFNAR1, IRF7, IFIH1, TLR3, TBK1, IRF3, TICAM1, UNC93B1, IFNAR2, STAT1, STAT2, TRAF3 or IRF9;
   (ii) IFNAR1, IRF7, IFIH1, TLR3, TBK1, IRF3, TICAM1, UNC93B1, IFNAR2, STAT1, STAT2 or TRAF3;
   (iii) IFNAR1, IRF7, IFIH1, TLR3, TBK1, IRF3, TICAM1, UNC93B1 and IFNAR2; and
   (iv) IFNAR1, IRF7, IFIH1, TLR3, TBK1, IRF3, TICAM1 and UNC93B1;
(c) wherein a patient or individual determined to have a LOF mutation is administered one or more Type I IFN to replace the function lost due to the mutation and/or is administered an immune-modulatory agent that increases or facilitates type I IFN-mediated response.

The invention further provides a method for evaluating a patient or individual positive for or at risk for SARS-CoV-2 infection or having COVID-19 disease for the presence of a loss-of-function (LOF) variation or mutation in the X-linked TLR7 gene and thereby determining treatment and treating the patient or individual comprising:
(a) isolating a blood or serum sample from said patient or individual;
(b) evaluating the blood or serum sample for a LOF variation or mutation in the TLR7 gene;
(c) wherein a patient or individual determined to have a LOF mutation is administered one or more Type I IFN to replace the function lost due to the mutation and/or is administered an immune-modulatory agent that increases or facilitates type I IFN-mediated response.

The invention further provides a method for evaluating a patient or individual positive for or at risk for SARS-CoV-2 infection or having COVID-19 disease for the presence of a loss-of-function (LOF) variation or mutation in a type I IFN pathway or response relevant gene and thereby determining treatment and treating the patient or individual comprising:
(a) isolating a blood or serum sample from said patient or individual;
(b) evaluating the blood or serum sample for a LOF variation or mutation in one or more type I IFN pathway gene selected from:
   (i) TLR7, IFNAR1, IRF7, IFIH1, TLR3, TBK1, IRF3, TICAM1, UNC93B1, IFNAR2, STAT1, STAT2, TRAF3 or IRF9;
   (ii) TLR7, IFNAR1, IRF7, IFIH1, TLR3, TBK1, IRF3, TICAM1, UNC93B1, IFNAR2, STAT1, STAT2 or TRAF3;
   (iii) TLR7, IFNAR1, IRF7, IFIH1, TLR3, TBK1, IRF3, TICAM1, UNC93B1 and IFNAR2;
   (iv) TLR7, IFNAR1, IRF7, IFIH1, TLR3, TBK1, IRF3, TICAM1 and UNC93B1; and
   (v) IFNAR1, IRF7, IFIH1, TLR3, TBK1, IRF3, TICAM1 and UNC93B1;
(c) wherein a patient or individual determined to have a LOF mutation is administered one or more Type I IFN to replace the function lost due to the mutation and/or is administered an immune-modulatory agent that increases or facilitates type I IFN-mediated response.

The invention further provides a method for evaluating a patient or individual prior to vaccination with live attenuated vaccine (LAV), such as LAV against yellow fever virus or COVID-19 or SARsCoV-2 virus, or having vaccine-associated disease, such as yellow fever vaccine-associated disease or COVID-19 vaccine-associated disease for the presence of a loss-of-function (LOF) variation or mutation in a type I IFN pathway gene and thereby determining whether the patient or individual can be safely vaccinated or should be vaccinated and/or treating the patient or individual comprising:
(a) isolating a blood or serum sample from said patient or individual;
(b) evaluating the blood or serum sample for a LOF variation or mutation in one or more type I IFN pathway gene selected from IFNAR1 and IFNAR2;
(c) wherein a patient or individual determined to have a LOF mutation is not vaccinated and/or is administered one or more Type I IFN to replace the function lost due to the mutation and/or is administered an immune-modulatory agent that increases or facilitates type I IFN-mediated response.

In an embodiment of the method, the patient or individual determined to have a LOF mutation is administered a Type I IFN.

In an embodiment of the method, the patient or individual determined to have a LOF mutation is administered IFN-α2 or IFN-β.

In an embodiment of the method, the variation or mutation in one or more type I IFN pathway gene is selected from a mutation provided in TABLE 1 or TABLE 2. In an embodiment of the method, the variation or mutation in one or more type I IFN pathway or IFN response relevant gene is selected from a mutation provided in TABLE 1, TABLE 2, TABLE 11 or TABLE 15.

In an embodiment of the method, the variation or mutation in one or more type I IFN pathway gene selected from TABLE 1 or TABLE 2 is determined via a primer or oligonucleotide specific for the mutation. In an embodiment of the method, the variation or mutation in one or more type I IFN pathway or IFN response relevant gene selected from TABLE 1, TABLE 2, TABLE 11 or TABLE 15 is determined via a primer or oligonucleotide specific for the mutation.

In an embodiment of the method, the variation or mutation in one or more type I IFN pathway gene selected from TABLE 1 or TABLE 2 is determined via a detectably labeled primer or oligonucleotide specific for the mutation, wherein hybridization and detection of the labeled primer or oligonucleotide is diagnostic for the presence of the mutation and LOF of the type I IFN in the patient or individual. In an embodiment of the method, the variation or mutation in one or more type I IFN pathway or response relevant gene selected from TABLE 1, TABLE 2, TABLE 11 or TABLE 15 is determined via a detectably labeled primer or oligonucleotide specific for the mutation, wherein hybridization and detection of the labeled primer or oligonucleotide is diagnostic for the presence of the mutation and LOF of the type I IFN in the patient or individual.

In an embodiment of the method, the variation or mutation in one or more type I IFN pathway gene selected from TABLE 1 or TABLE 2 is determined via whole genome or whole exome sequencing. In an embodiment of the method, the variation or mutation in one or more type I IFN pathway or response relevant gene selected from TABLE 1, TABLE 2, TABLE 11 or TABLE 15 is determined via whole genome or whole exome sequencing.

The invention further provides a method for evaluating a patient or individual positive for or at risk for SARS-CoV-2 infection or having COVID-19 disease for response to SARS-CoV-2 infection comprising:
(a) isolating plasmacytoid dendritic cells (pDCs) from the patient or individual;
(b) contacting the isolated pDCs with SARS-CoV-2 virus; and
(c) assessing the production of type I IFNs by the pDCs in response to SARS-CoV-2;
wherein reduced production of type I IFNs indicates that the patient or individual is altered in response to virus and is at high risk for severe COVID-19 disease.

In an embodiment of the method, the method further includes thereby determining treatment and treating the patient or individual.

In an embodiment of the method, the method further includes administering to the patient or individual the type I IFN or one or more type I IFN for which production is reduced.

In an embodiment, the patient is suspected of or first determined to carry or have family history of a variation or mutation in one or more type I IFN pathway or response relevant gene selected from TABLE 1, TABLE 2, TABLE 11 or TABLE 15.

In an embodiment, the patient is suspected of or first determined to carry or have family history of a variation or mutation in one or more type I IFN pathway or response relevant gene selected from TLR7, IFNAR1, IRF7, IFIH1, TLR3, TBK1, IRF3, TICAM1, UNC93B1, IFNAR2, STAT1, STAT2, TRAF3 or IRF9.

Other objects and advantages will become apparent to those skilled in the art from a review of the ensuing detailed description, which proceeds with reference to the following illustrative drawings, and the attendant claims.

### BRIEF DESCRIPTION OF THE DRAWINGS

The patent or patent application contains at least one drawing executed in color. Copies of this patent or patent application publication with color drawing(s) will be provided by the U.S. Patent and Trademark Office upon request and payment of the necessary fee.

FIG. 1 Functional test of IRF7, TLR3, TBK1, and IRF3 alleles detected in patients with severe COVID-19. (A) Luciferase reporter assays were performed in HEK293T cells transfected with empty vectors (EV), wildtype (WT), or candidate variants, and known LOF variants (F410V and Q321*). Firefly (IFNB reporter) luciferase activity was normalized against Renilla luciferase activity (housekeeping). Cells were either left unstimulated or were stimulated with Sendai virus 24 hours after transfection with IRF7 variants, and were subjected to luciferase assays 24 hours later. (B) Real-time PCR quantification of IFNL1 induction in TLR3-deficient P2.1 fibrosarcoma cells none-transfected (NT), or transfected with empty vector (EV), wildtype TLR3 (WT), candidate variants, or known LOF (W769*) and hypomorphic (R867Q) controls. Cells were either left unstimulated or were stimulated with poly(I:C) 24 hours after transfection with TLR3 variants, and were incubated for another 4 hours before RNA isolation. Real-time cycle numbers were normalized to housekeeping and WT. (C) Luciferase reporter assays were performed on HEK293T cells transfected with empty vectors (EV), wildtype (WT), candidate variants, hypomorphic control (D50A), and LOF control (G159A). Unstimulated cells were transfected with TBK1 for 48 hours before luciferase assay. (D) Luciferase reporter assays were performed in IRF3-deficient HEK293T cells transfected with empty vectors (EV), wildtype (WT), or candidate variants, and known LOF variant (R285Q). Firefly (IFNB reporter) luciferase activity was normalized against Renilla luciferase activity (housekeeping). Cells were either left unstimulated or were stimulated with Sendai virus 24 hours after transfection with IRF3 variants, and were subjected to luciferase assays 24 hours later.

FIG. 2 Functional test of TICAM1 and IFIH1 alleles detected in patients with severe COVID-19. (A) Luciferase reporter assays were performed in TICAM1-deficient fibroblast cells transfected with empty vectors (EV), wildtype (WT), or candidate variants, and known LOF variants (R141X and S186L). Firefly (IFNB reporter) luciferase activity was normalized against Renilla luciferase activity (housekeeping). (B) Cells were either left unstimulated or were stimulated with poly(I:C) 24 hours after transfection with IFIH1 variants, and incubated for another 20 hours before luciferase assay. The data shown are means±SD from 3 independent experiments. Relative luciferase activities for each variant were compared to WT MDA5 in one-way ANOVA with Dunnett's test for multiple comparisons****P<0.001, **P<0.01, *P<0.05.

FIG. 3 IRF7 expression in patients' PHA-T cell blasts. Cycling PHA-T cell blasts from patients were either left unstimulated or stimulated with 10,000 IU/ml IFN-α for 24 hours. Cell lysates were extracted with RIPA buffer with protease inhibitor cocktail and DNase, loaded on PVDF membrane and probe for IRF7 and MX1.

FIG. 4 Type I IFN responses to SARS-CoV2 in patients. (A-C) Type I IFN, type III IFN, and IL-6 production by pDCs purified from the AR IRF7 deficient patient. HD: healthy donor; ND: undetectable; IAV: Influenza A virus. (B) Serum levels of IFN-α in patients during acute COVID-19 were measured by Simoa. Detection limit of the essay was 0.01 pg/ml.

FIG. 5 Demographic and genetic inheritance of COVID-19 cohort. (A) Age and gender distribution of the COVID-19 patients. (B) Prediction of genetic inheritance model of genes under investigation.

FIG. 6 Functional test of TBK1 variants. Flag or HA tagged TBK1 were overexpressed in HEK293T cells. Total and phosphorylated TBK1, as well as total and phosphorylated IRF3 were measured by western blotting (A) when overexpressed, or (B) co-expressed with wildtype TBK1. (C) Luciferase reporter assays of TBK1 variants co-transfected with wildtype in HEK293T cells.

FIG. 7 Protein expression of TRAF3. DDK-tagged TRAF3 was over-expressed in HEK293T and protein level was measured by western blotting.

FIG. 8 Influenza virus protein microarrays (IVPM) in the serum of patients and controls. Frozen Belgian controls 1-10: age and gender matched controls living in the same region of Belgium as the patient.

FIG. 9 A, Multiplex particle-based assay for Auto-Abs against IFN-α2 and IFN-ω in patients with severe forms of COVID-19 (N=603). B, Multiplex particle-based assay for Auto-Abs against IFN-α2 and IFN-ω in healthy controls (N=350). C, Enzyme-linked immunosorbent assay (ELISA) for the 12 different IFN-α subtypes. D, Representative FACS plots showing the neutralizing effect on pSTAT1 in healthy donor cells in the presence of plasma from patients with auto-Abs against IFN-α and/or IFN-ω; E, Plotting of anti-IFN-α auto-Abs levels versus their neutralization ability. F, Neutralizing effect on ISG induction of plasma from 8 patients with auto-Abs against IFN-α2. G, Yellow fever vaccine strain 17D replication in cells treated with IFN-□ in the presence of control plasma, plasma from a patient with positive auto-Abs and a commercial anti-IFN-Q antibody. H, SARS-CoV-2 replication in cells treated with IFN-Q in the presence of control plasma, plasma from a patient with positive auto-Abs and a commercial anti-IFN-□ antibody. I, IFN-□ levels in the plasma or serum of patients with neutralizing Auto-Abs, as assessed by Simoa ELISA.

FIG. 10 A, Multiplex particle-based assay for Auto-Abs against IFN-α2 and IFN-ω in patients with severe forms of COVID-19 (N=603). B, Multiplex particle-based assay for Auto-Abs against IFN-α2 and IFN-ω in healthy controls (N=350). C, Representative FACS plots demonstrating no neutralizing effect on IFN-γ-induced pSTAT1 in healthy donor cells in the presence of plasma from a patient with auto-Abs against IFN-γ; D, Enzyme-linked immunosorbent assay (ELISA) for IFN-α2, IFN-ω, IL-22, and IL-17F showing the presence of autoantibodies in patients with severe forms of COVID-19; E, Enzyme-linked immunosorbent assay (ELISA) for IFN-α2, in healthy controls (N=7), asymptomatic COVID-19 individuals (N=138), Down syndrome patients (N=3), Incontinentia Pigmenti women (N=5) and an APS-1 patient (N=1). F, IgG, IgA, IgM and IgE auto-Abs levels against IFN-α2 in patients with positive anti-IFN-α2 auto-Abs. G, IgG, IgA, IgM and IgE auto-Abs levels against IFN-ω in patients with positive anti-IFN-α2 auto-Abs. H, Neighbor-joining phylogenetic tree of the 17 human type I IFN proteins. Horizontal branches are drawn to scale (bottom left, number of substitutions per site). Thinner, intermediate, and thicker internal branches have bootstrap support of <50, ≥50, and >80%, respectively. The bootstrap value for the branch separating IFN-w from all IFN-a subtypes is 100%. I, (A) ELISA for auto-Abs against the 13 different IFN-α subtypes, IFN-ω, IFN-β, IFN-κ, and IFN-ε in patients with life-threatening COVID-19 and auto-Abs against IFN-α2 (N=22), APS-1 patients (N=2), and healthy controls (N=2). J, LIPS for the 12 different IFN-α subtypes tested in patients with auto-Abs against IFN-α2 (N=22) and healthy controls (N=2).

FIG. 11: Homozygous loss-of-function variant of IFNAR2 in a patient with YELAVD. (A) Family pedigree showing the segregation of the IFNAR2 mutant (MT) allele. The proband is indicated by an arrow. E?: unknown IFNAR2 genotype. WT: wild-type. (B) Sanger sequencing results for IFNAR2 for the patient, her parents and healthy control leukocyte gDNA. (C) Exon trapping results demonstrating complete aberrant splicing in mutant IFNAR2-transfected COS-7 cells. At least 100 transcripts were sequenced for the patient and the control. (D) Schematic diagram of the full-length cDNA of the WT or MT IFNAR2. The exons are numbered by roman numerals (I-IX). The 5′ and 3′ UTR is shown in light gray and the coding sequences of the exons are shown in dark gray. (E) Schematic diagram of the WT IFNAR2 proteins, with the three known isoforms. The transmembrane domain is denoted “TM.” EC: extracellular domain. IC: intracellular domain. The mutation reported here is indicated in red and the previously reported mutation is indicated in violet. (F) Schematic diagram of the MT IFNAR2 proteins, with the impact on the three known isoforms. The shaded blue area shows the three amino acids resulting from the frameshift. (G) IFNAR2 mRNA levels, determined by RT-qPCR in HEK293T cells transiently transfected with IFNAR2 cDNA constructs; GUS was used as an expression control. NT: non-transfected, EV: empty vector, MT: mutant, WT: wild-type, p.E04fs110*: variant from the previously published IFNAR2−/− patient. Error bars represent standard deviation. (H) Western blot (WB) of IFNAR2 in HEK293T cells transiently transfected with IFNAR2 isoform c cDNA constructs. An antibody recognizing the V5 tag at the C-terminal end of the IFNAR2 protein was used. GAPDH was used as a loading control. A representative blot from two independent experiments is shown. (I) Graphical representation of extracellular FACS staining and the mean fluorescence intensity (MFI) of IFNAR2 in HEK293T cells transiently transfected with IFNAR2 cDNA constructs, using an antibody that recognizes the N-terminus of the protein. Cells were not permeabilized. Results representative of three independent experiments are shown. Error bars represent standard deviation. (J) Luciferase activity after IFN-α2 stimulation in IFNAR2−/− HEK293T cells generated with CRISPR/Cas-9 technology, and transiently transfected with IFNAR2 cDNA constructs (WT or MT). Results shown are from three cell lines generated with three independent CRISPR gRNAs. The bars represent the means and SEM of the results obtained with three cell lines generated with three different CRISPR gRNAs. Each dot correspond to the result obtained with one of these three cell lines.

FIG. 12: Neutralizing auto-Abs against IFN-α2 and IFN-ω in three patients with adverse reactions to yellow fever vaccine. (A) ELISA assay for auto-Abs against IFN-α2 and IFN-ω in three patients with life-threatening adverse reactions to yellow fever vaccine and healthy controls (N=200). O.D.: optical density. (B) Representative flow cytometry plots for IFN-α2- or IFN-ω-induced pSTAT1 in healthy control cells (gated on CD14+ monocytes) in the presence of 10% healthy control plasma or anti-IFN-α2, or anti-IFN-ω auto-Ab containing plasma from patients. Max, maximum; pos, positive; NS, not stimulated. (C) Neutralization effect on CXCL10 induction relative to GUS after the stimulation of healthy control PBMCs with IFN-α2, IFN-ω, or IFN-γ, in the presence of plasma from either healthy controls (N=2), two patients with adverse reactions to yellow fever vaccine and auto-Abs (P2 and P3), or one APS-1 patient. (D) Auto-Abs against the different type I IFN subtypes. ELISA for auto-Abs against the 13 different IFN-α subtypes, IFN-ω, IFN-β, IFN-κ, and IFN-ε in the three patients with adverse reactions to yellow fever vaccine and auto-Abs against IFN-α2, autoimmune polyendocrine syndrome type I (APS-1) patients (N=2), and healthy controls (N=2).

FIG. 13: Enhanced YFV replication, despite the presence of IFN-α2, in the presence of plasma from patients with auto-Abs against IFN-α2. YFV-17D replication, assessed 72 hours after infection, in Huh7.5 cells treated with IFN-α2 in the presence of plasma from either patients with life-threatening adverse reactions to YFV and neutralizing auto-Abs against IFN-α2 (N=2, P2 and P3), or a commercial anti-IFN-α2 antibody; or plasma from patients with adverse events following YFV vaccination but without auto-Abs against type I IFNs (N=2). Error bars represent standard error of the mean (SEM).

FIG. 14 provides additional data on the homozygous loss-of-function variant of IFNAR2 in a patient with YEL-AVD.

FIG. 15 provides additional data on the neutralizing auto-Abs against IFN-α2 and IFN-ω in three patients with adverse reactions to yellow fever vaccine.

FIG. 16 shows enhanced YFV replication, despite the presence of IFN-α2, in the presence of plasma from patients with life-threatening COVID-19 and auto-Abs against IFN-α2.

FIG. 17. Neutralizing auto-Abs against IFN-α2 and/or IFN-ω in patients with life-threatening COVID-19. (A) Gyros (high-throughput automated ELISA) results for auto-Abs against IFN-α2 and IFN-ω in patients with life-threatening COVID-19 (N=2,248), and in patients with asymptomatic or mild SARS-CoV-2 infection (N=588). (B) Schematic representation of the neutralization assay developed in HEK293T cells, using a luciferase system. ISRE: interferon-sensitive response elements. (C) Results for the neutralization of 10 ng/mL IFN-α2 or IFN-ω in the presence of 10% plasma from patients with life-threatening COVID-19 (N=2,036), or controls with asymptomatic infection (N=342). Relative luciferase activity is shown (ISRE dual luciferase activity, with normalization against Renilla luciferase activity) after stimulation with 10 ng/mL IFN-α2 or IFN-ω in the presence of 10% plasma. RLU: relative luciferase units. ISRE: interferon-sensitive response elements. (D) Relative luciferase activity (ISRE dual luciferase activity, with normalization against Renilla luciferase activity) after stimulation with IFN-α2 at a concentration of 10 ng/ml or 100 pg/mL, with various dilutions of plasma from a positive control (from 1/10 to 1/10⁷) neutralizing 10 ng/mL of type I IFNs (C+, 10 ng/mL), a patient neutralizing 100 pg/mL of type I IFNs but not 10 ng/mL (C+, 100 pg/mL), and a healthy control (HC). (E) Neutralization of 100 pg/mL IFN-α2 or IFN-ω in the presence of 10% plasma from patients with life-threatening COVID-19 (N=2,248), or controls with asymptomatic infection (N=588). Relative luciferase activity is shown (ISRE dual luciferase activity, with normalization against Renilla luciferase activity) after stimulation with 10 ng/mL IFN-α2 or IFN-ω in the presence of 10% plasma. RLU: relative luciferase units. ISRE: interferon-sensitive response elements. (F) Plot showing luciferase (ISRE dual luciferase activity, with normalization against Renilla luciferase activity) induction after stimulation with 10 ng/mL or 100 pg/mL IFN-α2, for patients with life-threatening COVID-19. Dotted lines indicate neutralizing levels, defined as induction levels below 15% of the mean value for controls tested the same day. Patients with antibodies neutralizing both 10 ng/mL and 100 pg/mL IFN-α2 are shown in the bottom left corner, whereas the patients in the bottom right corner had antibodies capable of neutralizing only 100 pg/mL IFN-α2. (G) Plot showing luciferase (ISRE dual luciferase activity, with normalization against Renilla luciferase activity) induction after stimulation with 10 ng/mL or 100 pg/mL IFN-ω, for patients with life-threatening COVID-19. Dotted lines indicate neutralizing levels, defined as induction levels below 15% of the mean value for controls tested the same day. Patients with antibodies neutralizing both 10 ng/mL and 100 pg/mL IFN-ω are shown in the bottom left corner, whereas the patients in the bottom right corner had antibodies capable of neutralizing only 100 pg/mL IFN-ω.

FIG. 18. Enhanced SARS-CoV-2 replication, despite the presence of IFN-α2, in the presence of plasma from patients with auto-Abs neutralizing 100 pg/mL IFN-α2. (A) SARS-CoV-2 replication in Huh-7.5 cells untreated, or treated with ˜96 pg/mL or ˜384 pg/mL IFN-α2 in the presence of 1/100 plasma from patients with life-threatening COVID-19 and auto-Abs neutralizing 100 pg/mL IFN-α2 in plasma 1/100 (N=5); elderly individuals with auto-Abs neutralizing 100 pg/mL IFN-α2 in plasma 1/100 (N=2); patients with life-threatening COVID-19 and auto-Abs neutralizing 10 ng/mL IFN-□2 in plasma 1/100 (N=1); a commercial anti-IFN-□2 antibody; plasma from patients with life-threatening COVID-19 but without auto-Abs against IFN-□2 (N=3), or plasma from healthy controls without auto-Abs (N=3). Each dot represents a technical replicate. All experiments were done in triplicated. (B) ELISA (enzyme-linked immunosorbent assay) for auto-Abs against the 13 IFN-α forms, IFN-ω, IFN-β, IFN-ε and IFN-κ in patients with life-threatening COVID-19 and auto-Abs neutralizing 100 pg/mL IFN-α2 (N=6), patients with life-threatening COVID-19 and auto-Abs neutralizing 10 ng/mL IFN-α2 (N=1), and healthy controls (N=2). (C) Neutralization of 10 ng/mL or 100 pg/mL IFN-β in the presence of 10% plasma from patients with life-threatening COVID-19 (N=692), or a- or pauci-symptomatic controls (N=109). Relative luciferase activity is shown (ISRE dual luciferase activity, with normalization against Renilla luciferase activity). RLU: relative luciferase units. ISRE: interferon-sensitive response elements.

FIG. 19. Higher prevalence of neutralizing auto-Abs against type I IFNs in elderly patients with life-threatening COVID-19 and in men. (A) Violin plot of the age and sex distribution of the patients with life-threatening COVID-19 included in our expanded cohort (N=2,248). (B) Graph showing the IFN-α2 auto-Ab levels, assessed by Gyros, in patients with life-threatening COVID-19. Men and women are shown separately. (C) Proportion of patients with life-threatening COVID-19 positive for auto-Abs against IFN-α2 and/or IFN-ω, with the neutralization of 10 ng/mL interferon by 10% plasma, by decade, for both sexes. (D) Proportion of patients with life-threatening COVID-19 positive for auto-Abs against IFN-α2 and/or IFN-ω, with the neutralization of 10 ng/mL interferon by 10% plasma, by decade, for both men and women. (E) Proportion of patients with life-threatening COVID-19 positive for auto-Abs against IFN-α2 and IFN-ω capable of neutralizing 10 ng/mL interferon in 10% plasma, by decade of age, for both sexes. (F) Proportion of patients with life-threatening COVID-19 positive for auto-Abs against IFN-α2 and IFN-ω capable of neutralizing 10 ng/mL interferon in 10% plasma, by decade of age, for both men and women. (G) Proportion of patients with life-threatening COVID-19 positive for auto-Abs against IFN-α2 and/or IFN-ω capable of neutralizing 100 pg/mL interferon in 10% plasma, by decade of age, for both sexes. (H) Proportion of patients with life-threatening COVID-19 positive for auto-Abs against IFN-α2 and/or IFN-ω capable of neutralizing 100 pg/mL interferon in 10% plasma, by decade of age, in men and women. (I) Proportion of patients with life-threatening COVID-19 positive for auto-Abs against IFN-α2 and IFN-ω capable of neutralizing 100 pg/mL interferon in 10% plasma, by decade of age, for both sexes. (J) Proportion of patients with life-threatening COVID-19 positive for auto-Abs against IFN-α2 and IFN-ω capable of neutralizing 100 pg/mL interferon in 10% plasma, by decade of age, in men and women.

FIG. 20. Higher prevalence of neutralizing auto-Abs against type I IFNs in patients who died of COVID-19. (A) Violin plot of the age and sex distribution of the patients who died of COVID-19 included in our cohort (N=865). (B) Graph showing the IFN-α2 auto-Ab levels, assessed by Gyros, in patients who died of COVID-19. Men and women are shown separately. (C) Proportion of patients who died of COVID-19 positive for auto-Abs against IFN-α2 and/or IFN-ω, with the neutralization of 10 ng/mL interferon by 10% plasma, by decade, for both sexes. (D) Proportion of patients who died of COVID-19 positive for auto-Abs against IFN-α2 and/or IFN-ω, with the neutralization of 10 ng/mL interferon by 10% plasma, by decade, for both men and women. (E) Proportion of patients who died of COVID-19 positive for auto-Abs against IFN-α2 and IFN-ω capable of neutralizing 10 ng/mL interferon in 10% plasma, by decade of age, for both sexes. (F) Proportion of patients who died of COVID-19 positive for auto-Abs against IFN-α2 and IFN-ω capable of neutralizing 10 ng/mL interferon in 10% plasma, by decade of age, for both men and women. (G) Proportion of patients who died of COVID-19 positive for auto-Abs against IFN-α2 and/or IFN-ω capable of neutralizing 100 pg/mL interferon in 10% plasma, by decade of age, for both sexes. (H) Proportion of patients who died of COVID-19 positive for auto-Abs against IFN-α2 and/or IFN-ω capable of neutralizing 100 pg/mL interferon in 10% plasma, by decade of age, in men and women. (I) Proportion of patients who died of COVID-19 positive for auto-Abs against IFN-α2 and IFN-ω capable of neutralizing 100 pg/mL interferon in 10% plasma, by decade of age, for both sexes. (J) Proportion of patients who died of COVID-19 positive for auto-Abs against IFN-α2 and IFN-ω capable of neutralizing 100 pg/mL interferon in 10% plasma, by decade of age, in men and women.

FIG. 21. Neutralizing auto-Abs against IFN-α2 and/or IFN-ω are more prevalent in the elderly, particularly in men, in the general population. (A) Violin plot of the age and sex distribution of individuals from the general population (N=33,352). (B) Graph showing the IFN-α2 auto-Ab levels, assessed by Gyros, in individuals from the general population. Men and women are shown separately. (C) Proportion of individuals from the general population positive for auto-Abs against IFN-α2 and/or IFN-ω capable of neutralizing 10 ng/mL interferon in 10% plasma, by decade of age, for both sexes. (D) Proportion of individuals from the general population positive for auto-Abs against IFN-α2 and/or IFN-ω capable of neutralizing 10 ng/mL interferon in 10% plasma, by decade of age, for men and women. (E) Proportion of individuals from the general population positive for auto-Abs against IFN-α2 and IFN-ω capable of neutralizing 10 ng/mL interferon in 10% plasma, by decade of age, for both sexes. (F) Proportion of individuals from the general population positive for auto-Abs against IFN-α2 and IFN-ω capable of neutralizing 10 ng/mL interferon in 10% plasma, by decade of age, for men and women. (G) Plot showing luciferase (ISRE dual luciferase activity, with normalization against Renilla luciferase activity) induction after stimulation with 10 ng/mL or 100 pg/mL IFN-α2 in 816 individuals from the general population over the age of 60 years. Dotted lines indicate neutralizing levels, defined as induction levels below 15% of the mean value for controls tested the same day. Patients with antibodies neutralizing both 10 ng/mL and 100 pg/mL IFN-α2 are shown in the bottom left corner, whereas the patients in the bottom right corner had antibodies capable of neutralizing only 100 pg/mL IFN-α2. (H) Plot showing luciferase (ISRE dual luciferase activity, with normalization against Renilla luciferase activity) induction after stimulation with 10 ng/mL or 100 pg/mL IFN-α2 stimulation in 816 individuals from the general population over the age of 60 years. Dotted lines indicate neutralizing levels, defined as induction levels below 15% of the mean value for controls tested the same day. Patients with antibodies neutralizing both 10 ng/mL and 100 pg/mL IFN-ω are shown in the bottom left corner, whereas the patients in the bottom right corner had antibodies capable of neutralizing only 100 pg/mL IFN-ω. (I) Proportion of individuals from the general population positive for auto-Abs against IFN-α2 capable of neutralizing 100 pg/mL interferon in 10% plasma, by decade of age, for both sexes. (J) Proportion of individuals from the general population positive for auto-Abs against IFN-ω capable of neutralizing 100 pg/mL interferon in 10% plasma, by decade of age, for both sexes.

FIG. 22 Neutralizing auto-Abs against IFN-α2 and/or IFN-ω in patients with life-threatening COVID-19. (A) Gyros IFN-α2 and IFN-ω titers for various dilutions of plasma from a patient positive for auto-Abs against type I IFNs. (B) Plot of anti-IFN-α2 auto-Ab levels, as detected by ELISA, against the values obtained for the same sample with Gyros (N=48 samples). A simple linear regression analysis was performed (black line). R²=0.9197. R², coefficient of determination. (C) Plot of the capacity to neutralize IFN-α2, as determined in pSTAT1 assays on PBMCs against that determined in the luciferase assay. The stimulation index (stimulated over unstimulated) for plasma from each patient was normalized against that of healthy control plasma from the same experiment for the pSTAT1 assay and that of the mean induction of control plasma tested the same day for the luciferase assay. R²=0.6036. (D) Plot of the capacity to neutralize IFN-ω, as determined with the pSTAT1 assay on PBMCs, against that determined in the luciferase assay. The stimulation index (stimulated over unstimulated) for plasma from each patient was normalized against that of healthy control plasma from the same experiment for the pSTAT1 assay, whereas luciferase activity was normalized against the Renilla luciferase activity of control plasma tested the same day. R²=0.6175. (E) Plot of anti-IFN-α2 auto-Ab levels, as determined by Gyros, against their neutralization capacity at 10 ng/mL in the luciferase assay. The luciferase activity for the plasma from each patient was normalized against the mean induction of control plasma tested the same day for the luciferase assay. The horizontal dotted line indicates neutralizing levels, defined as induction levels below 15% of the mean value for controls tested the same day. (F) Luciferase induction (ISRE dual luciferase activity, with normalization against Renilla luciferase activity) after stimulation with various amounts of IFN-α2, in presence of 10% plasma. (G) Relative luciferase activity (ISRE dual luciferase activity, with normalization against Renilla luciferase activity) after stimulation with IFN-ω at a concentration of 10 ng/ml or 100 pg/mL, with various dilutions of plasma from a positive control (from 1/10 to 1/10⁷) neutralizing 10 ng/mL of type I IFNs (C+, 10 ng/mL), a patient neutralizing 100 pg/mL of type I IFNs but not 10 ng/mL (C+, 100 pg/mL), and a healthy control (HC). (H) Plot of anti-IFN-α2 (upper panel), or anti-IFN-ω (lower panel) auto-Ab levels assessed by Luminex, against their neutralization capacity, assessed by the pSTAT1 assays on PBMCs. The stimulation index (stimulated over unstimulated condition) for the plasma from each patient was normalized against that of healthy control plasma from the same experiment.

FIG. 23. Enhanced SARS-CoV-2 replication, despite the presence of IFN-α2, in the presence of plasma from patients with auto-Abs neutralizing 100 pg/mL IFN-α2. (A) ELISA for auto-Abs against IFN-β in patients with life-threatening COVID-19 (N=384) and asymptomatic controls (N=109).

FIG. 24. Higher prevalence of neutralizing auto-Abs against type I IFNs in elderly patients with life-threatening COVID-19 and in men. (A) Graph showing the IFN-ω auto-Ab levels, assessed by Gyros, in patients with life-threatening COVID-19. Men and women are shown separately. (B) Proportion of patients with life-threatening COVID-19 positive for auto-Abs against IFN-α2, with the neutralization of 10 ng/mL interferon by 10% plasma, by decade, for both sexes. (C) Proportion of patients with life-threatening COVID-19 positive for auto-Abs against IFN-α2, with the neutralization of 10 ng/mL interferon by 10% plasma, by decade, for both men and women. (D) Proportion of patients with life-threatening COVID-19 positive for auto-Abs against IFN-ω capable of neutralizing 10 ng/mL interferon in 10% plasma, by decade of age, for both sexes. (E) Proportion of patients with life-threatening COVID-19 positive for auto-Abs against IFN-ω, capable of neutralizing 10 ng/mL interferon in 10% plasma, by decade of age, for both men and women. (F) Proportion of patients with life-threatening COVID-19 positive for auto-Abs against IFN-α2 capable of neutralizing 100 pg/mL interferon in 10% plasma, by decade of age, for both sexes. (G) Proportion of patients with life-threatening COVID-19 positive for auto-Abs against IFN-α2 capable of neutralizing 100 pg/mL interferon in 10% plasma, by decade of age, in men and women. (H) Proportion of patients with life-threatening COVID-19 positive for auto-Abs against IFN-ω capable of neutralizing 100 pg/mL interferon in 10% plasma, by decade of age, for both sexes. (I) Proportion of patients with life-threatening COVID-19 positive for auto-Abs against IFN-ω capable of neutralizing 100 pg/mL interferon in 10% plasma, by decade of age, in men and women.

FIG. 25. Higher prevalence of neutralizing auto-Abs against type I IFNs in patients who died of COVID-19. (A) Graph showing the IFN-ω auto-Ab levels, assessed by Gyros, in patients who died of COVID-19. Men and women are shown separately. (B) Proportion of patients who died of COVID-19 positive for auto-Abs against IFN-α2, with the neutralization of 10 ng/mL interferon by 10% plasma, by decade, for both sexes. (C) Proportion of patients who died of COVID-19 positive for auto-Abs against IFN-α2, with the neutralization of 10 ng/mL interferon by 10% plasma, by decade, for both men and women. (D) Proportion of patients who died of COVID-19 positive for auto-Abs against IFN-ω capable of neutralizing 10 ng/mL interferon in 10% plasma, by decade of age, for both sexes. (E) Proportion of patients who died of COVID-19 positive for auto-Abs against IFN-ω capable of neutralizing 10 ng/mL interferon in 10% plasma, by decade of age, for both men and women. (F) Proportion of patients who died of COVID-19 positive for auto-Abs against IFN-α2 capable of neutralizing 100 pg/mL interferon in 10% plasma, by decade of age, for both sexes. (G) Proportion of patients who died of COVID-19 positive for auto-Abs against IFN-α2 capable of neutralizing 100 pg/mL interferon in 10% plasma, by decade of age, in men and women. (H) Proportion of patients who died of COVID-19 positive for auto-Abs against IFN-ω capable of neutralizing 100 pg/mL interferon in 10% plasma, by decade of age, for both sexes. (I) Proportion of patients who died of COVID-19 positive for auto-Abs against IFN-ω capable of neutralizing 100 pg/mL interferon in 10% plasma, by decade of age, in men and women.

FIG. 26. Neutralizing auto-Abs against IFN-α2 and/or IFN-ω are more prevalent in the elderly, particularly in men, in the general population. (A) Graph showing the IFN-ω auto-Ab levels, assessed by Gyros, in individuals from the general population. Men and women are shown separately. (B) Proportion of individuals from the general population positive for auto-Abs against IFN-α2 capable of neutralizing 10 ng/mL interferon in 10% plasma, by decade of age, for both sexes. (C) Proportion of individuals from the general population positive for auto-Abs against IFN-α2 capable of neutralizing 10 ng/mL interferon in 10% plasma, by decade of age, for men and women. (D) Proportion of individuals from the general population positive for auto-Abs against IFN-ω capable of neutralizing 10 ng/mL interferon in 10% plasma, by decade of age, for both sexes. (E) Proportion of individuals from the general population positive for auto-Abs against IFN-ω capable of neutralizing 10 ng/mL interferon in 10% plasma, by decade of age, for men and women. (F) Type I IFN auto-Abs, assessed by LIPS (Luciferase based immunoprecipitation assay) in an independent Estonian cohort of 703 elderly individuals from the general population. (G) Type I IFN auto-Abs, assessed by ELISA in an independent Japanese cohort of 1002 individuals of all ages from the general population. (H) Proportion of individuals from the general population positive for auto-Abs against IFN-α2 and/or IFN-ω, capable of neutralizing 100 pg/mL interferon in 10% plasma, by decade of age, for both sexes. (I) Proportion of individuals from the general population positive for auto-Abs against IFN-α2 and/or IFN-ω, capable of neutralizing 100 pg/mL interferon in 10% plasma, by decade of age, for men and women. (J) Proportion of individuals from the general population positive for auto-Abs against IFN-α2 and IFN-ω, capable of neutralizing 100 pg/mL interferon in 10% plasma, by decade of age, for both sexes. (K) Proportion of individuals from the general population positive for auto-Abs against IFN-α2 and/or IFN-ω, capable of neutralizing 100 pg/mL interferon in 10% plasma, by decade of age, for men and women. (L) Proportion of individuals from the general population positive for auto-Abs against IFN-α2, capable of neutralizing 100 pg/mL interferon in 10% plasma, by decade of age, for both sexes. (M) Proportion of individuals from the general population positive for auto-Abs against IFN-ω, capable of neutralizing 100 pg/mL interferon in 10% plasma, by decade of age, for men and women.

FIG. 27. Enrichment in rare TLR7 deleterious alleles among men with critical COVID-19 pneumonia. (A) Manhattan plot showing the results of the variant enrichment test for the 190 genes of the X chromosome with at least 5 patients carrying non-synonymous variants. The red line indicates the corresponding Bonferroni-corrected significance threshold. (B) Western blot of extracts from non-transfected HEK293T cells (mock), HEK293T cells transfected with pCMV6 empty vector (EV), the wild-type (WT) TLR7 allele, or one of the TLR7 variant alleles of interest. All extracts were probed with monoclonal antibodies specific for the leucine-rich repeats within the human TLR7 protein. (C) (D) Luciferase assay on HEK293T cells transfected with the pGL4.32 luciferase reporter construct and an expression vector for Renilla luciferase together with no vector (mock), EV, WT, or TLR7 variants: (C) 18 variants found in our cohort and eight previously reported variants, (D) 109 variants found in male individuals from the gnomAD database. After 24 hours, transfected cells were left untreated or were treated by incubation with 1 μg/mL R848 for 24 hours. The y-axis represents NF-κB transcriptional activity as a percentage of the WT. The x-axis indicates the alleles used for transfection. (E) Diagram showing the correlation between allele frequency and NF-κB activity (% of WT). The 18 variants from 19 patients with critical SARS-CoV-2 from our cohort are shown in red, the eight previously reported variants are shown in blue and the 109 variants found in the general population (allele frequency above 10′ in men) are shown in gray. Activity of all LOF/hypomorphic alleles compared to WT allele were statistically significance (one-way ANOVA, P<0.01).

FIG. 28. X-linked recessive TLR7 deficiency in 14 kindreds. (A) Pedigrees of the 14 kindreds. The mutations are indicated above each pedigree. Solid black symbols indicate patients with critical COVID, and solid gray symbols indicate mild/moderate cases. The genotype is indicated under each symbol, with M corresponding to the mutation found in each kindred. ‘+’ and ‘−’ indicate the presence and absence, respectively, of antibodies against SARS-CoV-2 in the serum of the individual. (B) Schematic representation of TLR7. The upper part represents the genomic organization of the TLR7 locus, with rectangles for the various exons of the gene, and exon numbers indicated within the rectangle. The bottom part shows the primary structure of TLR7. The N-terminal portion and the leucine-rich repeat containing 26 leucine residues are located in the lumen of the endosome, and TM indicates the transmembrane domain. The Toll/interleukin-1 (IL-1) receptor (TIR) domain is cytoplasmic. The deleterious mutations reported in this study are indicated. (C) TLR7 expression in unstimulated EBV-B cells from two patients with XR TLR7 deficiency (P9 and P11), the fathers of P9 and P11, and the mother of P9, and three healthy donors (Control 1 to 3), determined by western blotting with detection with a specific TLR7 antibody. (D) TNF production by XR TLR7-deficient EBV-B cells. Cells were either left untreated or were stimulated with 5 μg/mL imiquimod (gray), or 25 ng/mL PMA and 0.25 μM ionomycin (black) for 24 hours and TNF production were measured by ELISA. (E) TNF production in XR TLR7-deficient EBV-B cells re-expressing WT TLR7. EBV-B cells from a control, P11, or an UNC93-deficient patient, cultured in the presence of IRAK4 inhibitor ((PF06650833-5 μM) were transduced with lentiviral particles that were empty or contained the WT TLR7 or mutant TLR7 cDNA. The cells were incubated for 24 h without IRAK4 inhibitor and were then left untreated or were stimulated with 1 μg/mL R848 (light gray), 5 μg/mL imiquimod (dark gray), or 25 ng/mL PMA and 0.25 μM ionomycin (black) for 24 hours, and TNF production were measured by ELISA. Statistical tests were performed using one-way ANOVA (*: P<0.05).

FIG. 29. Type I IFN responses to TLR7 agonist in TLR7-deficient pDCs and leukocytes. (A) Frequencies of five leukocyte subsets, determined by CyTOF. Healthy donors (black rectangles), relatives not carrying deleterious TLR7 alleles (blue rectangles) and hemizygous TLR7 variant carriers (red rectangles) are depicted. (B) TLR7 and TLR8 expression in different leukocyte subsets, determined by flow cytometry for the healthy control (C1). The result for another healthy control (C2) is shown in Figure S3C. (C) IFN-α production in purified leukocyte subsets with and without stimulation with various TLR7, 8, or 9 agonists (1 μg/mL CL264, 100 ng/mL TL8-506, 1 g/mL R848, or 2 μM CpG-c) for 24 hours. The y-axis shows IFN-α production on a logarithmic scale. The red bar corresponds to pDCs. (D) pDCs isolated from healthy donors and TLR7-deficient patients (P7, P11) were either left untreated (medium) or were stimulated with CL264 or CpG-c, and the production of IFN-α2 and IL-6 was assessed with CBAs on the supernatant. (E) Dotplot showing pDC diversification into subsets S1, S2, and S3 from magnetically sorted blood. pDCs from a TLR7-deficient patient (P11) and a healthy relative (K.1.1) were cultured for 24 h with medium alone or with 1 μg/mL CL264 or 2 μM CpG-c.

FIG. 30. Type I IFN responses to SARS-CoV-2 infection in TLR7-deficient pDCs. (A) pDCs isolated from healthy relatives and TLR7-deficient patients (P7, P11) were either left untreated (ns) or were infected with SARS-CoV-2 for 24 hours. RNA profiles were then determined by RNAseq. Genes with expression >2.0-fold higher or lower in controls after stimulation or infection are plotted as the fold-change in expression. (B) Induction of the type I and III IFN genes from (A) infected with SARS-CoV-2 for 24 hours or stimulated with CpG. (C) pDCs isolated from healthy relatives and TLR7-deficient patients (P7, P11) were either left untreated (ns) or were infected with SARS-CoV-2 for 24 hours and the production of IFN-α2, IP-10, IL-6 and IL-8 was measured with CBAs on the supernatant.

FIG. 31. Ethnic information and TLR7 allele activity. (A) PCA on the patients with TLR7 deficiency. EUR, Europeans; AFR, Africans; AMR, Americans; EAS, East-Asians; SAS, South-Asians. Red dots represent TLR7 variant carriers, blue dots represent patients with critical COVID-19 pneumonia, green dots represent asymptomatic or paucisymptomatic individuals, and gray dots represent individuals from the 1,000 Genomes database. (B) Luciferase assay on HEK293T cells transfected with no vector (mock), EV, WT, or TLR7 variants, together with the pGL4.32 luciferase reporter construct and an expression vector for Renilla luciferase. After 24 hours, transfected cells were left untreated or were treated with 5 μg/mL imiquimod (upper panel) or 5 μg/mL CL264 (lower panel) for 24 hours. The y-axis shows NF-κB transcriptional activity as a percentage of WT. The x-axis indicates the alleles used for transfection. (C) Allele activity for the M854I; L988S double-variant allele of TLR7. NF-κB luciferase assay on HEK293T cells transfected with no vector (mock), EV, WT, or TLR7 variants, together with the pGL4.32 luciferase reporter construct and an expression vector for Renilla luciferase. Cells were stimulated with 1 μg/mL R848 (left), 5 μg/mL imiquimod (center), or 5 g/mL CL264 (right). (D) TLR7 protein levels for eight variants previously reported in COVID-19 patients (32, 33). (E) Schematic representation of CADD and allele frequency for TLR7 in the general population (>10⁻⁵ in men). Gray dots represent isomorphic variants, and black dots represent hypomorphic or LOF variants, as estimated in FIG. 1D. (F) TLR7 protein levels of all the variants reported in men in the general population.

FIG. 32. Levels of TNF induction in EBV-B cells derived from two patients with XR TLR7 deficiency. (A) TNF mRNA levels in EBV-B cells from TLR7-deficient patients (P9, P11). The cells were stimulated with 1 μg/mL R848 (light gray bars), 5 μg/mL imiquimod (gray bars), or 25 ng/mL PMA plus 0.25 μM ionomycin for 6 hours (black bars). The y-axis shows the fold-change in TNF mRNA levels relative to the non-stimulated state (white bars). (B) TNF production in XR TLR7-deficient EBV-B cells (P9, P11). Cells were treated as in (A) but incubated for 24 hours before ELISA to assess TNF production. Statistical tests were performed using one-way ANOVA (*: P<0.05).

FIG. 33. Analysis of peripheral blood mononuclear cells from TLR7-deficient men. (A) Frequencies of T, B, or NK cells determined by CyTOF. Red indicates TLR7-deficient patients (P1, P4, P7, P8, P11, G.II.5, K.II.2), blue indicates familial controls, and black indicates healthy controls. (B) tSNE plot, with flow cytometry analysis showing the distribution of the principal peripheral leukocyte populations in two healthy controls (C1, C2). (C) Expression of TLR7 and TLR8 in various sets of leukocytes, assessed by flow cytometry for the healthy control (C2). The result for the other healthy control (C1) is shown in FIG. 3B. Black indicates leukocyte subsets expressing TLR7 or TLR8. Gray indicates leukocyte subsets without TLR7 or TLR8 expression. TLR7 expression in pDCs is shown in red. (D) Type I and III interferon (IFN-β, IFN-λ1, IFN-ω) production in each leukocyte subset, assessed by ELISA. Cells were left unstimulated or were stimulated with 1 μg/mL CL264, 100 ng/mL TL8-506 (TL8), 1 μg/mL R848, or 2 μM CpG-c. IL-8 production was measured as a positive control. (E) Dot plot showing pDC diversification into S1, S2, and S3 subpopulations from magnetically sorted blood pDCs from a TLR7-deficient patient (P1) and a healthy relative. Cells were cultured for 24 h with medium alone or with 1 μg/mL CL264 or 2 μM CpG-c.

FIG. 34. Functional analysis in pDCs infected with SARS-CoV-2. (A) pDC-depleted leukocytes isolated from a healthy donor (HD) and TLR7-deficient patients (P7, P11) were either left untreated (ns) or were infected with SARS-CoV-2 for 24 hours. The RNA expression profile of these cells was determined by RNAseq. Genes with expression levels >2.0-fold higher or lower in controls after stimulation or infection are plotted (gray dots) as the fold-change in expression. Type I and type III interferons are represenmed as red and blue dots (unrelated to their fold-induction) (B) Zoom for induction of the type I, III, and 11 IFN genes (IFNA, -IFNB, IFNE, IFNK, IFNW, IFNLs, and IFNG) in cells infected with SARS-CoV-2 for 24 hours or stimulated with CpG, (C) Dotplot showing pDC diversification into S1, S2, and S3 subpopulations from magnetically sorted blood pDCs from a TLR7-deficient patient (P11) and a healthy relative after culture for 24 h with either medium or SARS-CoV-2 at a MOI of 1. (D) pDCs isolated from a healthy donor (HD) and TLR7-deficient patient (P7, P11) were left untreated (medium), stimulated with CpG-c or infected with SARS-CoV-2 for 24 hours and the production of all forms of IFN-α (IFNα1, α2, α4, α5, α6, α7, α8, α10, α13, α14, α16, α17 and α21) was measured by ELISA on the supernatant.

FIG. 35. Allele activity for the TLR8 variants found in our cohort. Luciferase assay on HEK293T cells transfected with no vector (mock), EV, WT, or TLR8 variants, together with the pGL4.32 luciferase reporter construct and an expression vector for Renilla luciferase. After 24 hours, transfected cells were left untreated or were treated with 1 μg/mL R848 (left panel) or 100 ng/mL TL8-506 (right panel) for 24 hours. The y-axis shows NF-κB transcriptional activity as a percentage of WT. The x-axis indicates the alleles used for transfection. TIR_del corresponds to a construct with deletion of the Toll/IL-1 receptor (TIR) domain, expected to be amorphic.

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Therefore, if appearing herein, the following terms shall have the definitions set out below.

### A. Terminology

The term “antibody” describes an immunoglobulin whether natural or partly or wholly synthetically produced. The term also covers any polypeptide or protein having a binding domain which is, or is homologous to, an antibody binding domain. CDR grafted antibodies are also contemplated by this term. An “antibody” is any immunoglobulin, including antibodies and fragments thereof, that binds a specific epitope. The term encompasses polyclonal, monoclonal, and chimeric antibodies. The term “antibody(ies)” includes a wild type immunoglobulin (Ig) molecule, generally comprising four full length polypeptide chains, two heavy (H) chains and two light (L) chains, or an equivalent Ig homologue thereof (e.g., a camelid nanobody, which comprises only a heavy chain); including full length functional mutants, variants, or derivatives thereof, which retain the essential epitope binding features of an Ig molecule, and including dual specific, bispecific, multispecific, and dual variable domain antibodies; Immunoglobulin molecules can be of any class (e.g., IgG, IgE, IgM, IgD, IgA, and IgY), or subclass (e.g., IgG1, IgG2, IgG3, IgG4, IgA1, and IgA2). Also included within the meaning of the term “antibody” are any “antibody fragment”.

An “antibody fragment” means a molecule comprising at least one polypeptide chain that is not full length, including (i) a Fab fragment, which is a monovalent fragment consisting of the variable light (VL), variable heavy (VH), constant light (CL) and constant heavy 1 (CH1) domains; (ii) a F(ab′)2 fragment, which is a bivalent fragment comprising two Fab fragments linked by a disulfide bridge at the hinge region; (iii) a heavy chain portion of an Fab (Fd) fragment, which consists of the VH and CH1 domains; (iv) a variable fragment (Fv), which consists of the VL and VH domains of a single arm of an antibody, (v) a domain antibody (dAb) fragment, which comprises a single variable domain (Ward, E. S. et al., Nature 341, 544-546 (1989)); (vi) a camelid antibody; (vii) an isolated complementarity determining region (CDR); (viii) a Single Chain Fv Fragment wherein a VH domain and a VL domain are linked by a peptide linker which allows the two domains to associate to form an antigen binding site (Bird et al, Science, 242, 423-426, 1988; Huston et al, PNAS USA, 85, 5879-5883, 1988); (ix) a diabody, which is a bivalent, bispecific antibody in which VH and VL domains are expressed on a single polypeptide chain, but using a linker that is too short to allow for pairing between the two domains on the same chain, thereby forcing the domains to pair with the complementarity domains of another chain and creating two antigen binding sites (WO94/13804; P. Holliger et al Proc. Natl. Acad. Sci. USA 90 6444-6448, (1993)); and (x) a linear antibody, which comprises a pair of tandem Fv segments (VH-CH1-VH-CH1) which, together with complementarity light chain polypeptides, form a pair of antigen binding regions; (xi) multivalent antibody fragments (scFv dimers, trimers and/or tetramers (Power and Hudson, J Immunol. Methods 242: 193-204 9 (2000)); (xii) a minibody, which is a bivalent molecule comprised of scFv fused to constant immunoglobulin domains, CH3 or CH4, wherein the constant CH3 or CH4 domains serve as dimerization domains (Olafsen T et al (2004) Prot Eng Des Sel 17(4):315-323; Hollinger P and Hudson P J (2005) Nature Biotech 23(9):1126-1136); and (xiii) other non-full length portions of heavy and/or light chains, or mutants, variants, or derivatives thereof, alone or in any combination.

As antibodies can be modified in a number of ways, the term “antibody” should be construed as covering any specific binding member or substance having a binding domain with the required specificity. Thus, this term covers antibody fragments, derivatives, functional equivalents and homologues of antibodies, including any polypeptide comprising an immunoglobulin binding domain, whether natural or wholly or partially synthetic. Chimeric molecules comprising an immunoglobulin binding domain, or equivalent, fused to another polypeptide are therefore included.

The term “comprise” generally used in the sense of include, that is to say permitting the presence of one or more features or components.

The term “consisting essentially of” refers to a product, such as a peptide sequence, of a defined number of residues which is not covalently attached to a larger product.

The term “oligonucleotide,” as used herein in referring to a probe of use the present invention, is defined as a molecule comprised of two or more ribonucleotides, preferably more than three. Its exact size will depend upon many factors which, in turn, depend upon the ultimate function and use of the oligonucleotide. The term “primer” as used herein refers to an oligonucleotide, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The term “agent” means any molecule, including polypeptides, antibodies, polynucleotides, chemical compounds and small molecules. In particular the term agent includes compounds such as test compounds or drug candidate compounds.

The term “assay” means any process used to measure a specific property of a protein or of a compound. A “screening assay” includes a process used to characterize or select proteins or polypeptides based upon their activity, which may include signalling or downstream protein or response activity. A “screening assay” includes a process used to characterize or select compounds based upon their activity from a collection of compounds.

The term “preventing” or “prevention” refers to a reduction in risk of acquiring or developing a disease or disorder (i.e., causing at least one of the clinical symptoms of the disease not to develop) in a subject that may be exposed to a disease-causing agent, or predisposed to the disease in advance of disease onset. The term “prophylaxis” is related to and encompassed in the term ‘prevention’, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

“Therapeutically effective amount” means that amount of a drug, compound, antibody, or pharmaceutical agent that will elicit the biological or medical response of a subject that is being sought by a medical doctor or other clinician. In particular, with regard to gram-positive bacterial infections and growth of gram-positive bacteria, the term “effective amount” is intended to include an effective amount of a compound or agent that will bring about a biologically meaningful decrease in the amount of or extent of disease or flare free time period and or increase in length of a subject's survival or period disease-free or in remission or free of flare(s). The phrase “therapeutically effective amount” is used herein to mean an amount sufficient to prevent, and preferably reduce by at least about 30 percent, more preferably by at least 50 percent, most preferably by at least 90 percent, a clinically significant change, or enhanced survival or disease-free period by at least about 30 percent, more preferably by at least 50 percent, most preferably by at least 90 percent.

The term “treating” or “treatment” of any disease, condition, or infection refers, in one embodiment, to ameliorating the disease or infection (i.e., arresting the disease or growth of the infectious agent or bacteria or reducing the manifestation, extent or severity of at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or infection, either physically, (e.g., stabilization of a discernible symptom), physiologically, (e.g., stabilization of a physical parameter), or both. In a further embodiment, “treating” or “treatment” relates to slowing the progression of a disease or reducing an infection.

The phrase “pharmaceutically acceptable” refers to molecular entities and compositions that are physiologically tolerable and do not typically produce an allergic or similar untoward reaction, such as gastric upset, dizziness and the like, when administered to a human.

As used herein, “pg” means picogram, “ng” means nanogram, “ug” or “μg” mean microgram, “mg” means milligram, “ul” or “μl” mean microliter, “ml” means milliliter, “1” means liter.

### Abbreviation List

YFV: Yellow fever virus,
AR: Autosomal recessive,
YEL-AVD: Yellow fever vaccine-associated viscerotropic disease,
YEL-AND: Yellow fever vaccine-associated neurological disease,
SLE: Systemic lupus erythematosus,
IFNs: Interferons,
MMR: Measles-mumps-rubella,
HSE: Herpes simplex encephalitis,
WES: Whole-exome sequencing,
PCA: Principal component analysis,
IEI: Inborn errors of immunity,
SNVs: Single-nucleotide variants,
CNVs: copy number variants,
MAF: Minor allele frequency,
CADD: Combined annotation-dependent depletion,
MSC: Mutation significance cutoff,
gDNA: genomic DNA,
mRNA: messenger RNA,
MT: Mutant,
WB: Western blot,
KO: Knock-out,
ICU: Intensive care unit,
PBMC: Peripheral blood mononuclear cell

### B. Detailed Disclosure

The invention relates to and provides evaluable and inborn errors of type I IFN immunity which are associated with and identifiable in patients with severe COVID-19, the illness caused by SARS-CoV-2 infection. Variations in type I IFN-related autosomal genes have been identified in patients with life-threatening COVID-19 pneumonia. In accordance with the studies and results provided herein, mutations in one or more of IFNAR1, IRF7, IFIH1, TLR3, TBK1, IRF3, TICAM1 and UNC93B1 genes can be identified in patients with severe COVID-19 and are associated with and causal to aspects of severe COVID-19 disease. In an embodiment, mutations in IFNAR1, IRF7, IFIH1, TLR3, TBK1, IRF3, TICAM1, UNC93B1 and IFNAR2 can be identified in patients with severe COVID-19 and are associated with and causal to aspects of severe COVID-19 disease. In another embodiment, mutations in IFNAR1, IRF7, IFIH1, TLR3, TBK1, IRF3, TICAM1, UNC93B1, IFNAR2, STAT1, STAT2 or TRAF3 can be identified in patients with severe COVID-19 and are associated with and causal to aspects of severe COVID-19 disease. In another embodiment, mutations in IFNAR1, IRF7, IFIH1, TLR3, TBK1, IRF3, TICAM1, UNC93B1, IFNAR2, STAT1, STAT2, TRAF3 or IRF9 can be identified in patients with severe COVID-19 and are associated with and causal to aspects of severe COVID-19 disease. Identification of any one or more of these mutations in an individual positive for SARS-CoV-2 infection provides critical information that the individual is altered in type I IFN response and mots vulnerable to severe disease. Such an individual or patient must be managed and treated differently and with particular and specific care so as to avoid severe disease and pneumonia.

The present invention provides methods, assays and kits for assessment and evaluation of individuals prior to vaccination with live attenuated virus vaccines, particularly including yellow fever vaccines and particularly including COVID-19 or SARs-CoV2 vaccines, to assess risk for vaccine-associated disease and adverse events, and for evaluation, treatment and management of patients who develop vaccine-associated disease. The invention provides methods and assays for identification and characterization of auto-antibodies against Type I IFNs that are correlated and linked with vaccine-associated disease. The invention further provides methods of diagnosing and determining altered response to live attenuated virus vaccines, particularly including yellow fever vaccines and particularly including COVID-19 or SARs-CoV2 vaccines, or susceptibility to yellow fever virus (YFV) vaccine- or to COVID-19 vaccine-associated disease and for applicable and suitable treatment.

The invention further relates to auto-antibodies which have been identified in patients with severe COVID-19, the illness caused by SARS-CoV-2 infection. In accordance with the studies and results provided herein, patients having auto-antibodies against one or more type I IFN are at significant risk of life-threatening COVID-19 disease. The presence of auto-antibodies against one or more type I IFN in a patient or individual diagnosed with SARS-CoV-2 infection is causative for life-threatening COVID-19 disease. In these patients or individuals, the type I IFN response is altered and/or ineffective and normal IFN-mediated control and response to viral infection is limited or ineffective. In a particular embodiment, patients having auto-antibodies against IFN-α2 and/or IFN-ω are at significant risk of severe COVID-19 disease. In an embodiment, the presence of auto-antibodies directed against or specific for IFN-α2 and/or IFN-ω is correlated with and causative for severe COVID-19 disease. In a particular embodiment, patients having neutralizing auto-antibodies against IFN-α2 and/or IFN-ω are at significant risk of severe COVID-19 disease. In an embodiment, the presence of neutralizing auto-antibodies directed against or specific for IFN-α2 and/or IFN-ω is correlated with and causative for severe COVID-19 disease. In another embodiment, patients having neutralizing auto-antibodies against IFN-α2, IFN-ω, IFN-α1, IFN-α6, IFN-α13, IFN-α14 and IFN-α16 are at significant risk of severe COVID-19 disease. In another embodiment, the presence of auto-antibodies directed against or specific for one or more of IFN-α2, IFN-ω, IFN-α1, IFN-α6, IFN-α13, IFN-α14 and IFN-α16 is correlated with and causative for severe COVID-19 disease. In an embodiment, patients having neutralizing auto-antibodies additionally against IFN-β are at significant risk of severe COVID-19 disease. In another embodiment, the presence of auto-antibodies additionally directed against or specific for IFN-β is correlated with and causative for severe COVID-19 disease. In an embodiment, patients or at risk individuals may also have antibodies directed against IFN-β In an embodiment, the presence of auto-antibodies additionally directed against or specific for IFN-κ is correlated with and causative for severe COVID-19 disease. In an embodiment, patients or at risk individuals may also have antibodies directed against IFN-κ.

The invention relates to and provides evaluable and inborn errors of type I IFN immunity which are associated with and identifiable in individuals with altered response to or susceptibility to live attenuated vaccines, particularly and such as LAV COVID-19 vaccine, yellow fever virus (YFV) live attenuated vaccine and yellow fever virus YFV-17D vaccination and COVID-19 vaccine, particularly COVID-19 live attanuated vaccine, and/or which have vaccine-associated disease, particularly including YEL-AVD or YEL-AND or COVID-19 vaccine-related disease. Variations in type I IFN-related autosomal genes have been identified in patients with life-threatening vaccine-associated disease, such as COVID-19 vaccine-associated or YFV vaccine-associated disease. In accordance with the studies and results provided herein, mutations in IFNAR2 or one or more of IFNAR2 and IFNAR1 genes can be identified in patients with vaccine associated disease particularly live attenuated vaccine-associated disease, such as with YFV vaccine-associated disease or with COVID-19 vaccine-associated disease and are associated with and causal to aspects of severe YFV vaccine-associated disease. In an embodiment, autosomal recessive complete IFNAR2 and/or IFNAR1 deficiency can be identified in patients with vaccine-associated disease, particularly LAV vaccine-associated disease, such as COVID-19 or YFV vaccine-associated disease, and are associated with and causal to aspects of severe vaccine-associated disease.

The invention further relates to auto-antibodies which have been identified in patients or individuals with vaccine-associated disease and are associated with and causal to aspects of severe vaccine-associated disease, including YFV vaccine-associated disease. In accordance with the studies and results provided herein, patients having auto-antibodies against one or more type I IFN are at significant risk of life-threatening vaccine-associated disease. In these patients or individuals, the type I IFN response is altered and/or ineffective and normal IFN-mediated control and response to viral infection, even the limited replication and infection associated with live attenuated virus vaccines and such vaccination, is limited or ineffective. In a particular embodiment, patients having auto-antibodies against IFN-α2 and/or IFN-ω are at significant risk of vaccine-associated disease, such as COVID-19 vaccine-associated disease or YFV vaccine-associated disease. In a particular embodiment, patients having auto-antibodies against IFN-α2 and/or IFN-ω and/or IFN-β are at significant risk of YFV vaccine-associated disease. In a particular embodiment, patients having auto-antibodies against IFN-α2 and/or IFN-ω and/or IFN-β are at significant risk of COVID-19 vaccine-associated disease, particularly LAV COVID-19 vaccine-associated disease. In an embodiment, the presence of auto-antibodies directed against or specific for IFN-α2 and/or IFN-ω is correlated with and causative for severe YFV disease, including YFV vaccine-associated disease. In a particular embodiment, patients having neutralizing auto-antibodies against IFN-α2 and/or IFN-ω are at significant risk of severe LAV vaccine-associated disease, including YFV and COVID-19 vaccine-associated disease. In an embodiment, the presence of neutralizing auto-antibodies directed against or specific for IFN-α2 and/or IFN-ω is correlated with and causative for severe COVID-19 disease. In a particular embodiment, patients having neutralizing auto-antibodies against IFN-α2 and/or IFN-ω and/or IFN-β are at significant risk of severe LAV vaccine-associated disease, including YFV vaccine-associated disease. In an embodiment, the presence of neutralizing auto-antibodies directed against or specific for IFN-α2 and/or IFN-ω and/or IFN-β is correlated with and causative for severe COVID-19 disease.

In another embodiment, patients having neutralizing auto-antibodies against IFN-α2, IFN-ω, IFN-β, IFN-α1/13, IFN-α14, IFN-α7 and/or IFN-α14 are at significant risk of severe YFV vaccine-associated disease and/or adverse reaction. In another embodiment, the presence of auto-antibodies directed against or specific for one or more of IFN-α2, IFN-ω, IFN-β, IFN-α1/13, IFN-α14, IFN-α7 and/or IFN-α14 is correlated with and causative for severe YFV vaccine-associated disease. In another embodiment, patients having neutralizing auto-antibodies against IFN-α2, IFN-ω, IFN-α1/13, IFN-α14, IFN-α7 and/or IFN-α14 are at significant risk of severe LAV vaccine-associated disease and/or adverse reaction. In an embodiment, patients having neutralizing auto-antibodies additionally against IFN-β are at significant risk of severe LAV vaccine-associated disease, such as with LAV YFV vaccines or with LAV COVID-19 vaccines and/or adverse reaction. In an embodiment, patients or at risk individuals may also have antibodies directed against one or more of IFN-α4, IFN-α5, IFN-α6, IFN-α8, IFN-α10, IFN-α16, IFN-α17 and IFN-α21. In an embodiment, patients or at risk individuals may also have antibodies directed against one or more of IFN-κ and IFN-α.

Type I interferons (IFNs) are key components of the immediate antiviral response and are critical for restricting viral replication and spread. Type I IFNs work through autocrine and paracrine type I IFN receptor (IFNAR) signalling. It has recently been reported that minimal amounts of type I IFNs have been detected in the peripheral blood or lungs of patients with severe COVID-19 (Blanco-Melo D et al (2020) Cell 181:1036-1045 (doi.org/10.1016/j.cell.2020.04.026); Hadjaj J et al (2020) Science 10.1126/science.abc6027). However, the kinetics and requirements of systemic and local immune system response and immune modulators, including IFN responses, during COVID-19 are unclear and largely unknown, as are the specific contribution and requirements of response, including IFN responses, as they may contribute to COVID-19 pathogenesis and disease severity. Understanding and recognition of aspects and IFN responses, particularly those of the type I IFNs, correlated, associated with or even causative of an altered or ineffective response to SARS-CoV-2 infection is important, if not essential, in order to best and most quickly and appropriately identify a COVID-19 patient at significant risk or likelihood of severe COVID-19 disease so as to monitor, treat or specifically manage them in terms of therapeutic approaches or agents.

Human type I IFNs are a large subgroup of interferon proteins that help regulate the activity of the immune system. The mammalian type I IFNs are designated IFN-α (alpha), IFN-β (beta), IFN-κ (kappa), IFN-ε (epsilon) and IFN-ω (omega). All type I IFNs bind a specific cell surface receptor complex the IFN-α receptor (IFNAR) that consists of IFNAR1 and IFNAR2 chains. The IFN-α proteins are produced mainly by plasmacytoid dendritic cells (pDCs) and are involved in innate immunity against viral infection. IFN-α proteins comprise 13 subtypes IFN-αl, IFN-α2, IFN-α4, IFN-α5, IFN-α6, IFN-α7, IFN-α8, IFN-α10, IFN-α13, IFN-α14, IFN-α16, IFN-α17 and IFN-α21. Several recombinant IFN proteins are approved as therapeutic proteins and available and in clinical use. Recombinant IFN-α is available as a thereapeutic protein as either Intron® A (interferon alfa-2b) or Roferon®-A (interferon alfa-2a) and is administered by subcutaneous, intravenous or intramuscular injection. Pegylated forms of IFN-α are also available particularly pegylated interferon alfa-2a (trade name Pegasys) and pegylated interferon alfa-2b (tradename Pegintron). Recombinant Interferon beta IFN-β is available as a therapeutic in several marketed and approved forms including Avonex (interferon beta 1a), Rebif (interferon beta 1a), Plegridy (peginterferon beta 1a), Betaferon (interferon beta 1b), Extavia (interferon beta 1b). Pegylated IFN-β is available as plegridy (Biogen).

The present invention has discovered that the presence of inborn errors of type I interferon immunity or auto-antibodies against Type I IFNs dictates and defines aspects and approaches to therapy for COVID-19 disease. In certain embodiments, the presence of loss of function recessive or dominant, homozygous or heretozygous mutations in type I IFN genes results in an altered and/or nonfunctional or ineffective immune or IFN-mediated response to virus infection, thereby resulting in pathological and severe COVID-19 disease with SARS-CoV-2 infection. Identification of the type I IFN gene mutation present in a patient permits and enables the specific and targeted therapy such as administering the IFN protein which function is lost to the patient.

The presence of auto antibodies directed against Type I IFNs results in an altered and/or nonfunctional or ineffective immune or IFN-mediated response to virus infection, thereby resulting in pathological and severe COVID-19 disease with SARS-CoV-2 infection. Patients with auto-antibodies against an IFN type will not respond to treatment with that IFN subtype, and in fact administration of that subtype could be harmful and dangerous. This issue compounded with the already compromised state of a COVID-19 patient is very serious. The recognition that up to 10% of severe COVID-19 patients have circulating neutralizing auto-Abs directed against specific IFN proteins now provides approaches and methods for identifying these patients, even and particularly at early stage of disease or upon initial recognition of SARS-CoV-2 infection so that they can be differentially managed and treated.

The progression of COVID-19 illness and serious disease can be significant and rapid. Any treatment which can specifically correct a recognized deficiency in a SARS-CoV-2 infected patient could have important impacts on a patient's course of disease and the need for critical care admission and treatment. For hospitalized patients with pneumonia, about 50% develop hypoxia by day 8 and severe illness and cytokine release syndrome develop generally within 5-10 days after symptom onset in susceptible patients. The availability of methods and approaches to evaluate and predict inborn errors of Type I immunity and/or auto-Abs against Type I IFN proteins could reduce the impact of if not eliminate aspects of cytokine release syndrome. Severe infection and severe COVID-19 disease can be characterized by and include the following markers: regular high fevers (greater than 102° F.), respiratory rate greater than 30 breaths per minute, worsening oxygen requirements (4-6L nasal cannula), elevated IL-6 levels (greater than 40-100), CRP, ferritin, d-dimer.

The invention provides a method for evaluating the presence of anti-type I IFN specific auto-antibodies (auto-Abs) in a patient or individual positive for or at risk for SARS-CoV-2 infection or having COVID-19 disease and thereby determining treatment and treating the patient or individual comprising:
(a) isolating a blood or serum sample from said patient or individual;
(b) evaluating the blood or serum sample for auto-antibodies specific for one or more type I IFN selected from:
   (i) IFN-α2 and IFN-ω;
   (ii) IFN-α2, IFN-ω and IFN-β;
   (iii) IFN-α2, IFN-ω, IFN-β and IFN-α;
   (iv) IFN-α2, IFN-ω, IFN-β, IFN-ε and IFN-κ;
   (v) IFN-α2, IFN-ω, IFN-β, IFN-ε, IFN-α1, IFN-α2, IFN-α6, IFN-α13, IFN-α14 and IFN-α16; and
   (vi) IFN-α2, IFN-ω, IFN-α1, IFN-α2, IFN-α6, IFN-α13, IFN-α14 and IFN-α16;
(c) wherein a patient or individual is treated to remove or deplete the auto-Abs and/or is administered a type I IFN against which they do not have auto-Abs and/or is administered an immune-modulatory agent that increases or facilitates type I IFN-mediated response.

In accordance with the method, the blood or serum sample may evaluated for neutralizing antibodies. The sample may be further evaluated for neutralizing antibodies, for example, once auto-Abs are identified. The blocking of downstream effects or specific activity of an IFN type or subtype can be assessed and determined. One skilled in the art will know and have available approaches to assess IFN-specific activity and mediated effects. Thus, as an example if auto-Abs are identified against IFN-α2, antibody blocking of IFN-α2-mediated activity can be evaluated. In an example, cells may be incubated with IFN-α2 and patient plasma and assessed for pSTAT1 induction. The instant examples describe neutralization assays which can be conducted.

In an embodiment, a method is provided for prevention of severe COVID-19 disease. Identification of existing auto-Abs in a patient and specific treatment or mediation directed to remove or reduct the auto-Abs or to administer an alternative IFN which is not neutralized, could prevent the onset of severe COVID-19 disease in a SARS-CoV-2 infected individual.

In an embodiment of the method, plasmapheresis is conducted on the patient or individual to deplete the antibodies, or wherein B cells, such as auto-reactive B cells, and/or plasmacytes or plasmablasts are depleted in the patient or individual. Plasmaphersis is a known and readily available process wherein plasma is separated from blood cells. The plasma may replaced with another solution such as saline or albumin, or the plasma is treated and then returned to the patient's body. Plasmapheresis is utilized clinically in various instances, including to remove antibodies from blood. Approaches to deplete B cells, such as auto-reactive B cells, plasmacytes or plasmablasts are known and available in the art. Monoclonal antibodies capable of depleting plasmablasts include daratumumab (Darzalex) or isatuximab (Sarclisa), which are directed against CD38. Rituximab (Rituxan, Truxima) is a chimeric monoclonal antibody directed against CD20, which is primarily found on the surface of immune system B cells, and triggers B cell death with binding. Approaches utilized to reduce antibody or B cell responses in auto-immune diseases for example may also be considered or utilized. Once auto-Abs are reduced or depleted, in an embodiment, IFN therapy and administration of for example IFN-α or another Type I IFN protein can proceed.

Alternatively, a patient may be treated with or administered an IFN agent or protein against which they do not have auto-Abs. A patient or individual having auto-Abs against one or more Type I IFN may be treated by administering an IFN subtype which is not neutralized by the patient's or individual's auto-Abs. A patient or individual having auto-Abs against one or more of Type I IFN may be treated by administering an IFN-α subtype which is not neutralized by the patient's or individual's auto-Abs. A patient or individual having auto-Abs against one or more of IFN-α2, IFN-ω, IFN-ε, IFN-α1, IFN-α2, IFN-α6, IFN-α13, IFN-α14 or IFN-α16 and not having auto-Abs against IFN-β may treated by administering IFN-β. A patient or individual having auto-Abs against one or more Type I IFN selected from IFN-ω, IFN-ε, IFN-β, IFN-κ, and not having auto-Abs against IFN-α2 is treated by administering IFN-α2. Suitable IFN-α and IFN-β for administration is known and available.

Diagnostic assays for evaluating the presence of type I IFN auto-Abs are provided and included in the invention. An assay for evaluating the presence of auto-antibodies directed against one or more Type I IFN in a patient or individual positive for or at risk for SARS-CoV-2 infection or having COVID-19 disease is provided comprising:
(a) contacting a sample of blood or serum from the patient or individual with one or more recombinant type I IFN protein selected from IFN-α2, IFN-ω, IFN-β, IFN-ε and IFN-κ, wherein each IFN protein is labeled with a distinct detectable tag or marker to form an antibody-protein complex;
(b) contacting any antibody-protein complex of (a) with one or more labeled anti-human immune globulin molecule that will bind and label auto-antibody bound to any one or more IFN protein;
(c) specifically and selectively detecting each type I IFN protein bound by specific auto-Ab thereto.

In the assay, one or more recombinant type I IFN protein may labeled with a fluorescent marker. One or more recombinant type I IFN protein may covalently coupled to a magnetic bead with a fluorescent marker. Each of the one or more recombinant type I IFN proteins may be covalently coupled to a magnetic bead with a distinct and differential fluorescent marker. The examples herein describe for example a multiplex particle-based assay for auto-Abs against IFN-α2 and IFN-ω. A suitable similar assay may be utilized or generated, particularly including all or most applicable IFNs so that a single test can identify any and all relevant auto-Abs in a patient.

The one or more labeled anti-human immune globulin molecule that will bind and label bound auto-antibody may a labeled non-human animal derived anti-human IgG. For example a labeled goat anti-Human IgG or labeled rabbit anti-human IgG may be utilized.

In particular embodiments, the one or more recombinant type I IFN protein may selected from IFN-α2, IFN-ω and IFN-β, or the one or more recombinant type I IFN protein may selected from IFN-α2 and IFN-ω.

In further embodiments of the assay, the presence of neutralizing auto-antibodies is determined. Whether an antibody is neutralizing can be determined utilizing any approaches or methods known and available to the skilled artisan. Neutralizing antibodies will block their target protein's activity. Neutralizing antibodies against IFN-α2, for example will eliminate STAT1 phosphorylation in response to IFN-α2.

In additional embodiments, the methods and assays of the invention are applicable to plasma samples for therapy, particularly convalescent plasma from patients that have recovered from virus infection. Convalescent plasma is being utilized for treatment of COVID-19 patients.

Evaluation of the plasma to assess and determine whether auto-Abs against type I IFNs are present will ensure that the plasma does not contain antibody blockers or neutralizers of any critical type I IFN. If antibodies are found, the applicable plasma sample can be discarded or can be treated or processed to remove the antibodies. For example, plasma auto-Abs can be removed by panning or otherwise contacting the plasma with the relevant type I IFN and binding out or removing the auto-Abs.

Systems and kits for evaluating and determining auto-Abs against type I IFNS are included as aspects of the invention. The invention provides a kit for for evaluating the presence of auto-antibodies directed against one or more Type I IFN in a patient or individual at risk of, suspected of or determined to have coronavirus comprising:
(a) one or more recombinant type I IFN protein selected from IFN-α2, IFN-ω, IFN-β, IFN-ε and IFN-κ, wherein each IFN protein is labeled with a distinct detectable tag or marker;
(b) one or more labeled anti-human immune globulin molecule that will bind and label auto-antibody bound to any one or more IFN protein;
(c) a means for specific and selective detection of each type I IFN protein bound by specific auto-Ab thereto.

In embodiments of a kit of the invention, one or more recombinant type I IFN protein may be labeled with a fluorescent marker. One or more recombinant type I IFN protein may be covalently coupled to a magnetic bead with a fluorescent marker. Alternatively, each of the one or more recombinant type I IFN proteins may covalently coupled to a magnetic bead with a distinct and differential fluorescent marker. This permits determination of any and all applicable anti-type I IFN auto-Abs in a single step, and indicates which, including if more than one, type I IFN is targeted by antibodies in the patient sample. Suitable fluorescent markers, including magnetic beads or such other suitable bead or selectable tag having fluorescent markers are known and available to one skilled in the art. Fluorescent markers may include for example fluorescein, rhodamine, Texas Red, green fluorescent protein, auramine, AMCA blue and Lucifer Yellow. The fluorescent protein may be selected from one or more of a blue/UV protein, a cyan protein, a green protein, a yellow protein, an orange protein, a red protein, a far-red protein, a near-IR protein, a long stokes shift protein, a photactivatible protein, a photoconvertible protein and a photo switchable protein. Examples of blue/UV fluorescent proteins include TagBFP and Sapphire. Examples of Cyan proteins include ECFP and derivatives thereof, Cerulean, TagCFP and mTFP1. Examples of green proteins include GFP and derivatives thereof, Emerald, monomeric azami green. Examples of yellow proteins include EYFP and derivatives thereof, and examples of orange proteins include monomeric kusabira orange and derivatives thereof. Red fluorescent proteins are known in the art and include for example RFP and derivatives thereof, mRaspberry, mCherry, mStrawberry, mRuby.

In an embodiment of the kit, the one or more labeled anti-human immune globulin molecule that will bind and label bound auto-antibody is a labeled non-human animal derived anti-human IgG. For example a labeled goat anti-Human IgG or labeled rabbit anti-human IgG may be utilized.

In embodiments of the kit, the one or more recombinant type I IFN protein may be selected from IFN-α2, IFN-ω and IFN-β, or may be selected from IFN-α2 and IFN-ω.

Numerous IFN gene variations and mutations are described and provided herein, wherein the mutations result in loss of function of an IFN. A listing of exemplary identified specific variations and mutations is provided herein, including in TABLE 1 and TABLE 2 and also in TABLE 11 and TABLE 15. The invention further provides a method for evaluating a patient or individual positive for or at risk for SARS-CoV-2 infection or having COVID-19 disease for the presence of a loss-of-function (LOF) variation or mutation in a type I IFN pathway gene and thereby determining treatment and treating the patient or individual comprising:
(a) isolating a blood or serum sample from said patient or individual;
(b) evaluating the blood or serum sample for a LOF variation or mutation in one or more type I IFN pathway gene selected from:
   (i) IFNAR1, IRF7, IFIH1, TLR3, TBK1, IRF3, TICAM1, UNC93B1, IFNAR2, STAT1, STAT2, TRAF3 or IRF9;
   (ii) IFNAR1, IRF7, IFIH1, TLR3, TBK1, IRF3, TICAM1, UNC93B1, IFNAR2, STAT1, STAT2 or TRAF3;
   (iii) IFNAR1, IRF7, IFIH1, TLR3, TBK1, IRF3, TICAM1, UNC93B1 and IFNAR2; and
   (iv) IFNAR1, IRF7, IFIH1, TLR3, TBK1, IRF3, TICAM1 and UNC93B1;
(d) wherein a patient or individual determined to have a LOF mutation is administered one or more Type I IFN to replace the function lost due to the mutation and/or is administered an immune-modulatory agent that increases or facilitates type I IFN-mediated response.

The invention further provides a method for evaluating a patient or individual positive for or at risk for SARS-CoV-2 infection or having COVID-19 disease for the presence of a loss-of-function (LOF) variation or mutation in a type I IFN pathway or response relevant gene and thereby determining treatment and treating the patient or individual comprising:
(a) isolating a blood or serum sample from said patient or individual;
(b) evaluating the blood or serum sample for a LOF variation or mutation in one or more type I IFN pathway or response relevant gene selected from:
   (i) TLR7, IFNAR1, IRF7, IFIH1, TLR3, TBK1, IRF3, TICAM1, UNC93B1, IFNAR2, STAT1, STAT2, TRAF3 or IRF9;
   (ii) TLR7, IFNAR1, IRF7, IFIH1, TLR3, TBK1, IRF3, TICAM1, UNC93B1, IFNAR2, STAT1, STAT2 or TRAF3;
   (iii) TLR7, IFNAR1, IRF7, IFIH1, TLR3, TBK1, IRF3, TICAM1, UNC93B1 and IFNAR2;
   (iv) TLR7, IFNAR1, IRF7, IFIH1, TLR3, TBK1, IRF3, TICAM1 and UNC93B1; and
   (v) IFNAR1, IRF7, IFIH1, TLR3, TBK1, IRF3, TICAM1 and UNC93B1;
(c) wherein a patient or individual determined to have a LOF mutation is administered one or more Type I IFN to replace the function lost due to the mutation and/or is administered an immune-modulatory agent that increases or facilitates type I IFN-mediated response.

The invention further provides a method for evaluating a patient or individual positive for or at risk for SARS-CoV-2 infection or having COVID-19 disease for the presence of a loss-of-function (LOF) variation or mutation in the X-linked TLR7 gene and thereby determining treatment and treating the patient or individual comprising:
(a) isolating a blood or serum sample from said patient or individual;
(b) evaluating the blood or serum sample for a LOF variation or mutation in the TLR7 gene;
(c) wherein a patient or individual determined to have a LOF mutation is administered TLR7 protein or one or more Type I IFN to replace the function lost due to the mutation and/or is administered an immune-modulatory agent that increases or facilitates type I IFN-mediated response.

In an embodiment of the method, the patient or individual determined to have a LOF mutation is administered a Type I IFN. In an embodiment of the method, the patient or individual determined to have a LOF mutation is administered IFN-α2 or IFN-β. As previously described herein and above, thereapeutic IFN-α2 and IFN-β proteins are known and commercially available for clinical use.

In an embodiment of the method, the variation or mutation in one or more type I IFN pathway gene is selected from a mutation provided in TABLE 1 or TABLE 2. In an embodiment of the method, the variation or mutation in one or more type I IFN pathway or response relevant gene is selected from a mutation provided in TABLE 1, TABLE 2, TABLE 11 or TABLE 15. TABLES 1 and 2 list and provide various mutations in genes IFNAR1, IRF7, IFIH1, TLR3, TBK1, IRF3, TICAM1, UNC93B1, IFNAR2, STAT1, STAT2, TRAF3 or IRF9 which have been identified and may be assessed in accordance with the method. TABLE 11 and TABLE 15 list and provide various mutations in genes, including X-linked genes, particularly including TLR7 gene, which have been identified and may be assessed in accordance with the method. The variation or mutation in one or more type I IFN pathway gene selected from TABLE 1 or TABLE 2 may be determined via a primer or oligonucleotide specific for the mutation. The variation or mutation in one or more type I IFN pathway or response relevant gene selected from TABLE 1, TABLE 2, TABLE 11 or TABLE 15 may be determined via a primer or oligonucleotide specific for the mutation.

In accordance with the present disclosure, the tables present information with which an ordinarily skilled practitioner can access the amino acid sequences of the proteins and nucleic acid sequences of the encoding genes identified herein as including loss of function variations and mutations. A stepwise protocol or means for identification of the sequences listed in the tables presented herein may include the artisan accessing one of the publicly available databases and entering a relevant ensembl number or gene name or symbol to identify the sequence and relevant marker information. Such information may be used to design probes for detection of any of the proteins, genes therein or to identify commercially available probes or antibodies therefore or thereof. Primers for detection of nucleic acid sequences encoding any of the proteins listed in the tables presented herein are also envisioned as are primers for PCR including RT PCR. Such primers may be used to detect RNA expression levels or to detect the presence of a specific variation or mutation in the nucleic acid. The design of primers for detecting the presence of a specific variation or mutation listed herein is a matter of routine practice with the nucleic acid sequence in hand as provided by publicly available websites such as those mentioned above. Such probes and primers are useful for the kits described herein.

Human protein encoding sequences and amino acid sequences for the various referenced type I interferon (IFN) pathway proteins are known and available. For example, protein sequences in public databases are known and include the following: IRF7 (NP_004022.2), TLR3 (NP_003256.1), TBK1 (NP 037386.1, NM_013254.4), IRF3 (AAH09395), TICAM1 (NP_891549), IFNAR1 (NP_000620.2), UNC93B1 (NP_112192.2), IFIH1 (AAI11751.1, NP_071451.2), IFNAR2 (NP_001276054.1), IRF9 (NP_001372329.1), STAT1 (NP_009330.1), STAT2 (NP 005410.1), TRAF3 (NP_003291.2) and TLR7 (NP_057646.1; AAZ99026.1).

In an embodiment of the method, the variation or mutation in one or more type I IFN pathway gene selected from TABLE 1 or TABLE 2 is determined via a detectably labeled primer or oligonucleotide specific for the mutation, wherein hybridization and detection of the labeled primer or oligonucleotide is diagnostic for the presence of the mutation and LOF of the type I IFN in the patient or individual. In an embodiment of the method, the variation or mutation in one or more type I IFN pathway or response relevant gene selected from TABLE 1, TABLE 2, TABLE 11 or TABLE 15 is determined via a detectably labeled primer or oligonucleotide specific for the mutation, wherein hybridization and detection of the labeled primer or oligonucleotide is diagnostic for the presence of the mutation and LOF of the type I IFN in the patient or individual.

In an embodiment of the method, the variation or mutation in one or more type I IFN pathway gene selected from TABLE 1 or TABLE 2 is determined via whole genome or whole exome sequencing. In an embodiment of the method, the variation or mutation in one or more type I IFN pathway or response relevant gene selected from TABLE 1, TABLE 2, TABLE 11 or TABLE 15 is determined via whole genome or whole exome sequencing. The examples describe sequencing to determine and assess the mutations. One skilled in the art can readily undertake and conduct sequencing, even targeted gene region sequencing to identify or screen for one or more variations or mutations, including loss of function mutations, in the IFN response relevant genes identified and provided herein.

In other embodiments of the invention and methods provided herein, a patient or individual may first be screened or evaluated for the presence and/or levels of type I IFN in their blood, plasma or serum. In a particular embodiment, a patient or individual may first be screened or evaluated for the presence and/or levels of IFN-α in their blood, plasma or serum. As detailed herein, IFN-α was undetectable in the serum of the patients with type I IFN auto-Abs on the first days of hospitalization. Also, serum levels of the 13 IFN-α in patients with LOF were reduced and at levels under the detection limit in many/a majority of the LOF mutation patients during the acute phase of COVID-19 infections. Thus assays and methods are provided comprising first evaluating a patient or individual for levels of type I IFNs, in a particular aspect for levels of the 13 IFN-α proteins; in the event that IFN-α levels, levels are low or undetectable, the patient or individual is evaluated for mutations in type I IFNs. Thus assays and methods are provided comprising first evaluating a patient or individual for levels of type I IFNs, in a particular aspect for levels of IFN-α; in the event that IFN levels, particularly in an aspect IFN-α levels are low or undetectable, the patient or individual is evaluated for auto-Abs against type I IFNs. The patient can be evaluated for auto-Abs against (i) IFN-α2 and IFN-ω; (ii) IFN-α2, IFN-ω and IFN-β; (iii) IFN-α2, IFN-ω, IFN-β and IFN-ε; (iv) IFN-α2, IFN-ω, IFN-β, IFN-ε and IFN-κ; (v) IFN-α2, IFN-ω, IFN-β, IFN-ε, IFN-α1, IFN-α2, IFN-α6, IFN-α13, IFN-α14 and IFN-α16; or (vi) IFN-α2, IFN-ω, IFN-α1, IFN-α2, IFN-α6, IFN-α13, IFN-α14 and IFN-α16. The patient or individual thereby identified as having auto-Abs is identified as likely to progress to severe COVID-19 disease and is treated to remove or deplete the auto-Abs and/or is administered a type I IFN against which they do not have auto-Abs and/or is administered an immune-modulatory agent that increases or facilitates type I IFN-mediated response.

The studies provided herein demonstrate that in certain patients or individuals, particularly those with severe COVID-disease, including wherein the severe disease is unexpected given patient history or relatively young age, have mutations in genes in the type I IFN pathway or response relevant genes. Dendritic cells, particularly plasmacytoid dendritic cells (pDCs), isolated from these patients or individuals are demonstrated to have altered (reduced) type I IFN response when incubated with SARS-CoV-2 virus or subject to SARS-CoV-2 infection. Thus, in an aspect of the invention, dendritic cells, particularly plasmacytoid dendritic cells (pDCs), can be isolated from a patient or individual and assessed for Type I IFN response, such as for production of one or more Type I IFN or production of IFNα subtypes, upon incubation with or infection by SARS-CoV-2. The patient or individual may or may not have been determined to have a gene mutation or be at risk for a gene mutation, including such as is provided herein including in Table 1, Table 2, Table 11 and/or Table 15. The invention provides a method for evaluating a patient or individual positive for or at risk for SARS-CoV-2 infection or having COVID-19 disease for response to SARS-CoV-2 infection comprising:
(e) isolating plasm, acytoid dendritic cells (pDCs) from the patient or individual;
(f) contacting the isolated pDCs with SARS-CoV-2 virus; and
(g) assessing the production of type I IFNs by the pDCs in response to SARS-CoV-2;
wherein reduced production of type I IFNs indicates that the patient or individual is altered in response to virus and is at high risk for severe COVID-19 disease.

In an embodiment of the method, the method further includes thereby determining treatment and treating the patient or individual. In an embodiment of the method, the method further includes administering to the patient or individual the type I IFN or one or more type I IFN for which production is reduced. In an embodiment, reduced production of one or IFNα subtype protein by the pDCs of a patient or individual is determined. In a further embodiment, the IFNα subtype protein which is reduced or absent is administered to the patient or individual to aid in response and recovery from COVID-19. The IFN a subtype may be selected from IFNα1, α2, α4, α5, α6, α7, α8, α10, α13, α14, α16, α17 and α21.

In an embodiment, the patient is suspected of or first determined to carry or have family history of a variation or mutation in one or more type I IFN pathway or response relevant gene selected from TABLE 1, TABLE 2, TABLE 11 or TABLE 15. In an embodiment, the patient is suspected of or first determined to carry or have family history of a variation or mutation in one or more type I IFN pathway or response relevant gene selected from TLR7, IFNAR1, IRF7, IFIH1, TLR3, TBK1, IRF3, TICAM1, UNC93B1, IFNAR2, STAT1, STAT2, TRAF3 or IRF9.

The methods provided herein have applicability to other virus infections, particularly respiratory viruses and respiratory viral disease. Thus, the methods of the invention are applicable in patients suspected of or at risk of infection with rhinovirus, respiratory syncytial virus, influenza virus, coronavirus, parainfluenza virus and/or adenovirus. The invention is applicable prior to vaccination with a live or live attenuated vaccine. The examples describe serious disease for example following vaccination with yellow fever vaccine, which is a live vaccine. Thus, with development, implementation and administration of a live or live attenuated COVID-19/SARS-CoV-2 vaccine, testing of individuals prior to vaccination, particularly to evaluate the presence if neutralizing auto-Abs to type I IFN(s) would be beneficial and very important. Further, in instances where a patient may have recovered from severe COVID-19 infection, it would be beneficial to assess the presence of auto-Abs against type I IFNs and/or the presence of one or more LOF mutation in the IFN-related autosomal genes described herein so as to have that information available in further instances of any viral exposure or infection. In addition, relatives of those whe have experienced or succumbed to COVID-19 disease should be evaluated, particularly in view of the autosomal recessive mutations that lead to severe COVID-19 disease. In as much as the presence of auto-Abs and even of LOF mutations have been generally not evident by way of clinical manifestations even prior to SARS-CoV-2 infection, family relatives or children may also be carrying one or more mutation(s) or may have auto-Abs.

The methods and approaches described herein are applicable to LAVs other than yellow fever, including MMR, and also LAVs in development or under consideration for coronavirus, particularly Sars-Cov-2. Several LAVs are presently in development for Sars-Cov-2. For example, Sanchez-Felipe L et al 2020 describe the development of a candidate vaccine (YF-S0) for severe acute respiratory syndrome coronavirus 2 (SARS-CoV-2) that uses live-attenuated yellow fever 17D (YF17D) vaccine as a vector to express a noncleavable prefusion form of the SARS-CoV-2 spike antigen.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### Example 1

### Inborn Errors of Type I IFM Immunity in Patients with Severe Covid-19

There is immense inter-individual clinical variability upon infection with SARS-CoV-2, ranging from silent infection to lethal COVID-19. We searched for variations in 13 type I IFN-related autosomal genes previously shown to define monogenic etiologies of relatedviral diseases. We found an enrichment in rare variants predicted to be loss-of-function (LOF) at the 13 human loci known to govern Toll-like receptor 3 (TLR3)- and interferon regulatory factor 7 (IRF7)-dependent type I interferon (IFN) immunity to influenza virus in patients with life-threatening COVID-19 pneumonia relative to subjects with asynptomatic or benign infection. We found biochemically deleterious variations at 8 loci in 76 of 682 patients with life-threatening COVID-19 pneumonia. These mutations underlie both known recessive (IFNAR1, IRF7) or dominant deficiencies (IFIH1, TLR3, TBK1, IRF3, TICAM1), and novel dominant deficiencies (UNC93B1, IFNAR1, IRF7). About 60% patients tested had undetectable levels of serum IFN-α in the course of COVID. Moreover, fibroblasts heterozygous or homozygous for loss-of-function (LOF) variations in IFIH1, TLR3, or IRF7 were vulnerable to SARS-CoV-2 infection. Finally, plasmacytoid dendritic cells from the patients heterozygous or homozygous for a LOF variation in IRF7 failed to produce type I IFNs in response to SARS-CoV-2. Inborn errors of type I IFN production can underlie life-threatening COVID-19 pneumonia at various ages. Treatment with type I IFN early in the course of SARS-CoV-2 infection may be beneficial in selected patients.

SARS-CoV-2 is responsible for the COVID-19 pandemic, which has already claimed at least 600,000 deaths (1). Most life-threatening cases begin with a pneumonia, which progresses to acute respiratory distress syndrome (ARDS) and the failure of other organs (2-4). SARS-CoV-2 infection has been diagnosed by pharyngeal PCR in over 14 million people, most of whom had mild, self-healing disease (1). The global number of infected individuals is at least 10 times higher, as suggested by recent serological and virological studies (5). The clinical manifestations of infection with SARS-CoV2 thus range from silent infection to lethal disease (6-8). Globally, infection-fatality rate ranges from 0.1% to 0.9% (9). Two epidemiological factors increase the risk of severity, whether defined as hospitalization or death. The most striking is age; risk increases sharply, decade by decade, after the age of 50 years (6, 10). Various underlying medical conditions constitute a second risk factor (11). Following on our human genetic studies of other severe infectious diseases performed since 1996 (12, 13), we established the COVID Human Genetic Effort (https://www.covidhge.com) to test the hypothesis that severe COVID-19 may be caused, especially but not exclusively, in relatively young and previously healthy patients, by monogenic inborn errors of immunity to SARS-CoV-2, more often with incomplete than complete penetrance (14, 15).

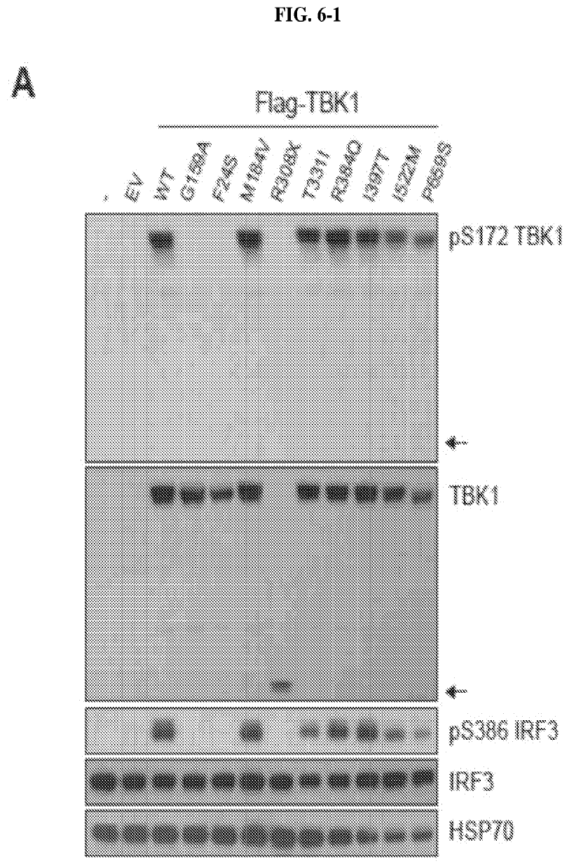

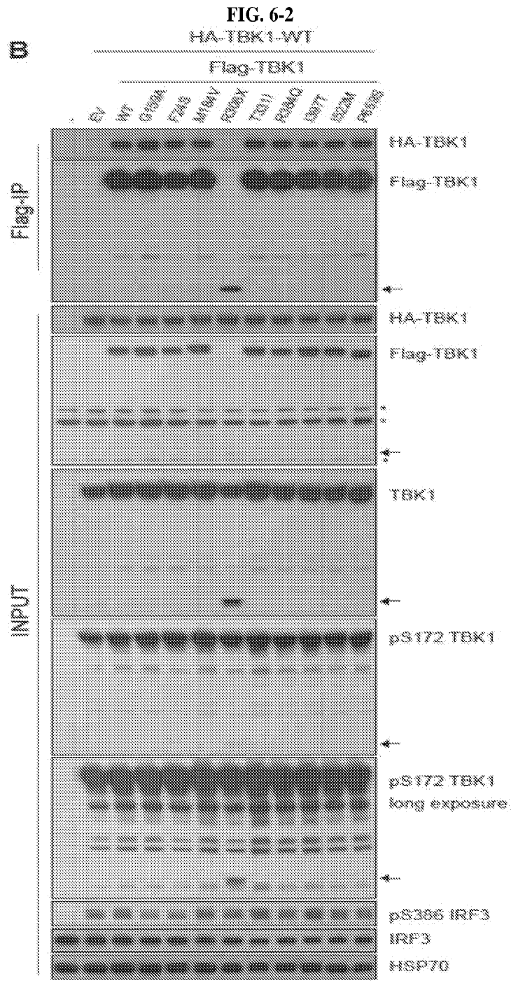

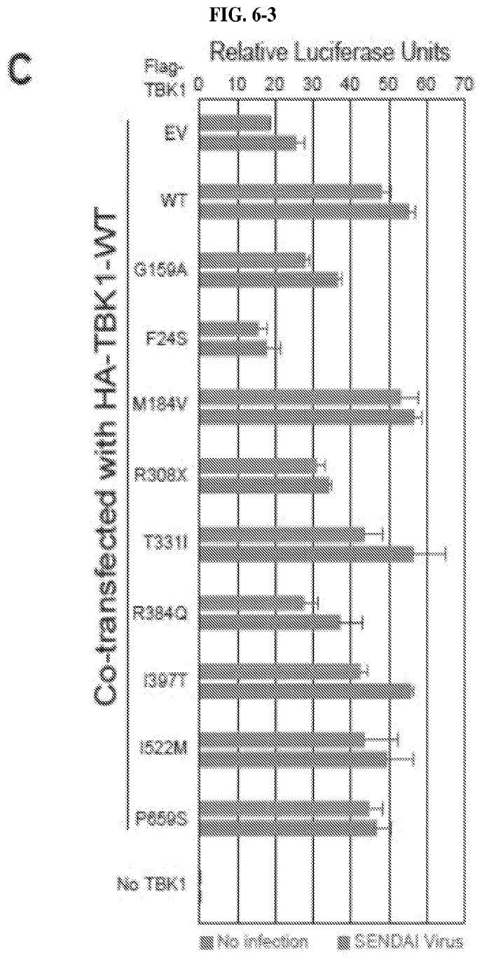

We tested the hypothesis that known autosomal inborn errors of Toll-like receptor 3 (TLR3)- and interferon regulatory factor 7 (IRF7)-dependent type I interferon (IFN) immunity previously shown to underlie severe respiratory viral diseases (mutations in IFIH1 (16-18) for rhinovirus and respiratory syncytial virus, and mutations in TLR3 (19), IRF7 (20-22), IRF9 (23) for influenza virus) or shown to underlie non-respiratory viral illnesses but predicted to underlie severe respiratory viral diseases (mutations in TICAM1 (24), UNC93B1 (25), TRAF3 (26) in the TLR3 pathway, mutations in TBK1 (27) and IRF3 (28) in the TLR3 and IFIH1 pathways, and mutations in IFNAR1 (29), IFNAR2 (30), STAT1 (31), STAT2 (32) in the IRF7- and IRF9-dependent pathway), may also underlie severe COVID-19 pneumonia. We enrolled 682 patients (498 men and 184 women) living in various countries and aged 1 month to 91 years (FIG. 6A). All patients were hospitalized for life-threatening pneumonia due to SARS-CoV-2 (inclusion and exclusion criteria see methods). We sequenced their whole genome (281) or exome (401). We searched for both mono- and bi-allelic variations in all 13 loci, despite that only autosomal recessive (AR) inheritance model has been previously reported in IRF7-, IRF9-, IFNAR2-, and STAT2-deficiencies. We considered novel genetic forms, especially autosomal dominant (AD) inheritance models, at these loci because none of the patients developed severe infections to other common respiratory virus prior to COVID-19, consistent with a higher virulence of SARS-CoV-2 and possible incomplete penetrance of these defects.

Under an AR (autosomal recessive) model, we found four COVID-19 patients with five bi-allelic variants of IRF7 (homozygous p.Pro364fs and p.Met371Val/p.Asp117Asn) and IFNAR1 (homozygous p.Trp73Cys and homozygous p.Ser422Arg). Under an AD (autosomal dominant) model, we found 194 patients carrying 157 candidate variants of TLR3 (N=7), IRF7 (N=16), TBK1 (N=13), IRF3 (N=6), TICAM1 (18), TRAF3 (N=8), IFIH1 (N=36), IRF9 (N=4), IFNAR1 (N=11), IFNAR2 (N=13), STAT1 (N=2), STAT2 (N=15), and UNC93B1 (N=8). (Table S1). Overall, we found 162 candidate variants (3 homozygous, 2 compound heterozygous, and 157 heterozygous) in 198 patients (TABLE 2). Among the candidate variants, 18 were predicted loss-of-function (pLOF) whereas the remaining 144 were missense or small in-frame indels (TABLE 3). Remarkably, these variants included one that had previously been reported in patients with life-threatening influenza infection and herpes simplex encephalitis (TLR3 p.Pro554Ser) (16, 19) and another previously shown to be biochemically deleterious (IFNAR1 p.Pro335del) (33). We did not detect homozygous copy number variations (CNVs) in these 13 genes.

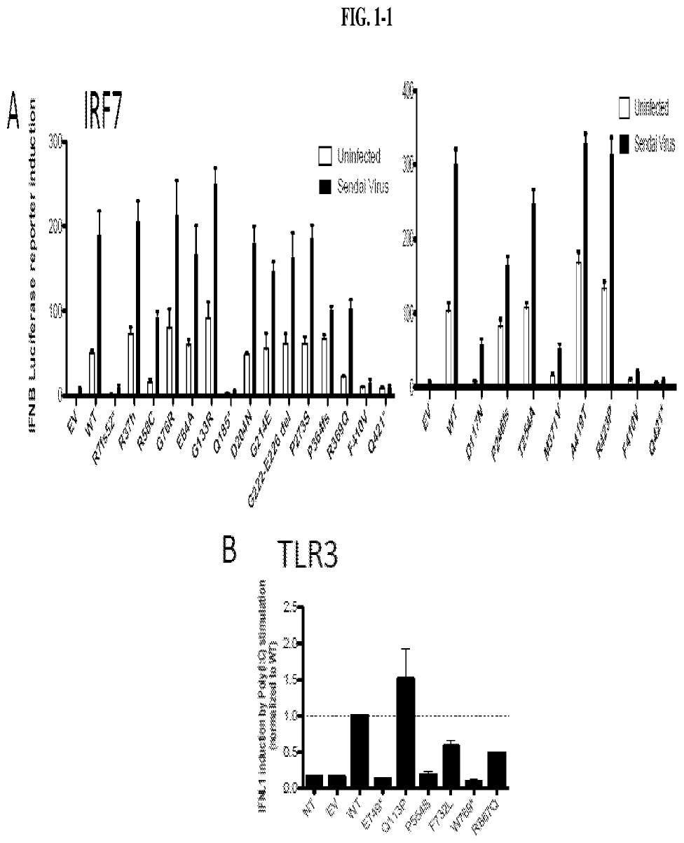

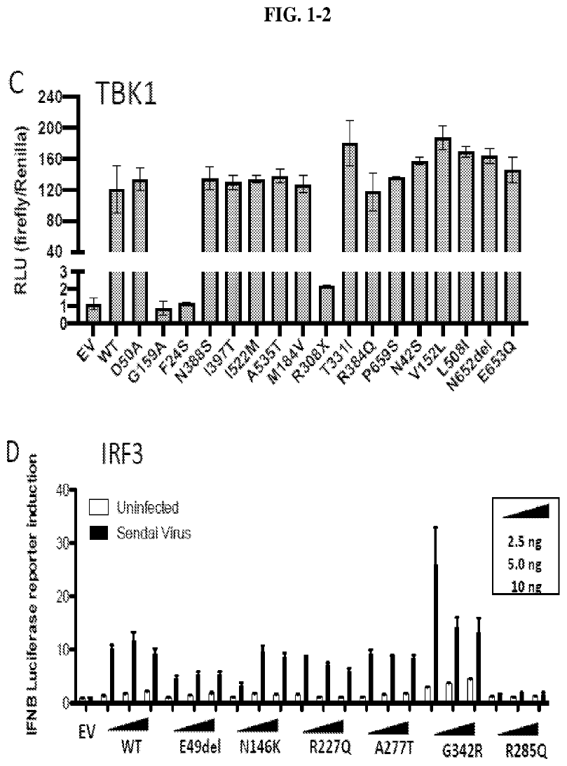

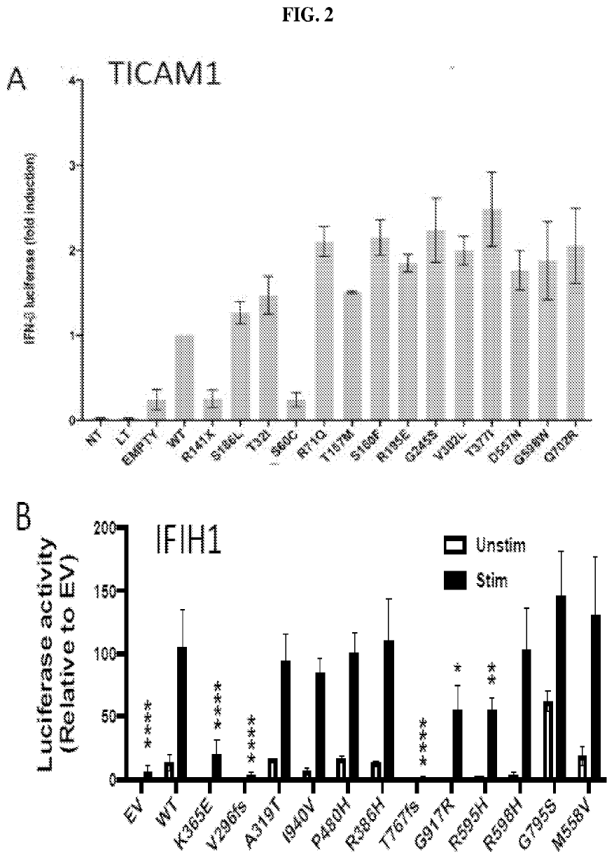

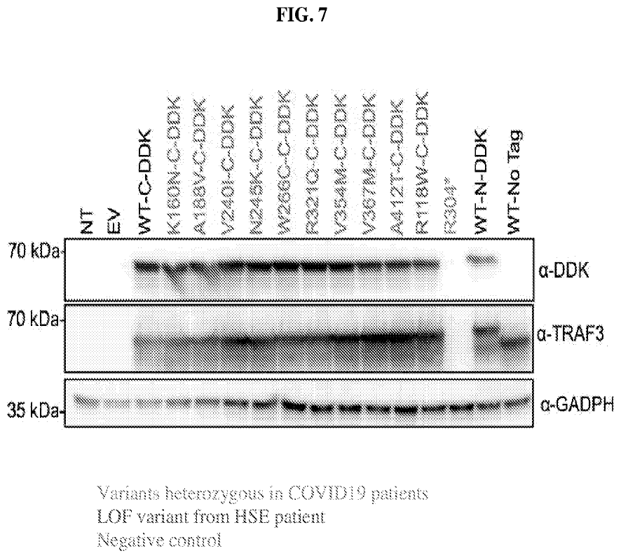

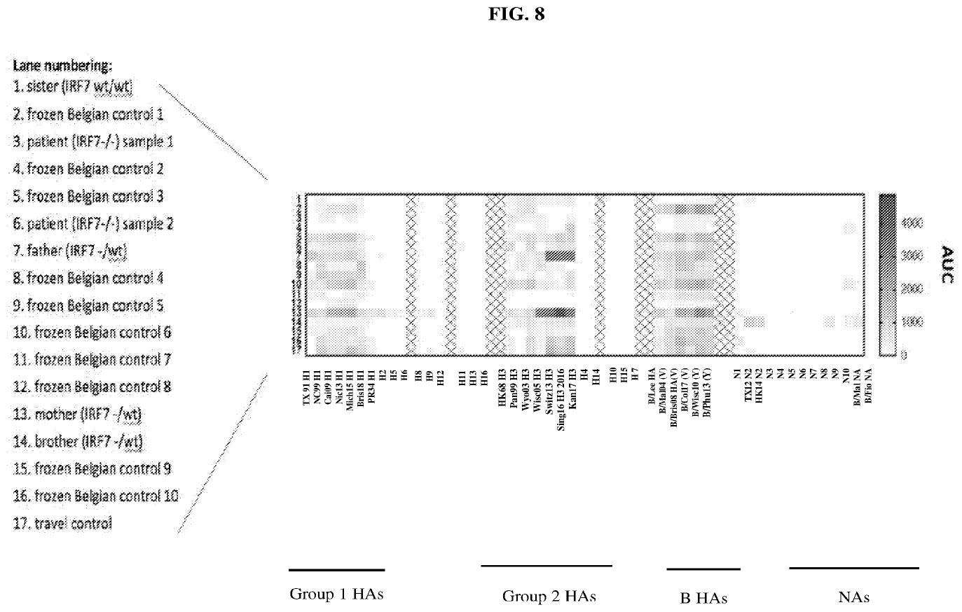

We then set off to test the expression and function of each of these variants in ad hoc overexpression systems. We found that 37 variants were LOF (or hypomorphic), in IRF7 (p.Pro364fs, p.Met371Val, p.Asp117Asn, p.Gln185*, p.Pro246fs, p.Arg7fs, p.Arg369Gln), TLR3 (p.Pro554Ser, p.Ser339fs, p.Trp769*), TBK1 (p.Arg308*, p.Phe24Ser, p.Arg384Gln), IRF3 (p.Asn146Lys, p.Glu49del), TICAM1 (p.Ser60Cys), IFNAR1 (p.Trp73Cys, p.Ser422Arg, p.Pro335del), UNC93B1 (p.Glu96*), and IFIH1 (Δexon14, Δexon8, p.Ala589fs, p.Arg728*, p.Cys565Phe, p.Cys910Ser, p.Glu627*, p.Ile923Val, p.Leu679fs, p.Lys365Glu, p.Met570Thr, p.Phe744Leu, p.Pro866Leu, p.Thr767fs, p.Tyr25*, p.Val296fs, p.Val929Ile) (TABLE 1, FIG. 1, FIG. 2, FIG. 7, FIG. 8). The remaining 125 variants tested were neutral in our assays. Seventy-six patients carried these 37 deleterious variants, resulting in 4 AR deficiencies (homozygosity for IRF7 p.Pro364fs, compound heterozygosity for IRF7 p.Met371Val/p.Asp117Asn, homozygosity for IFNAR1 p.Trp73Cys or p.Ser422Arg), 30 AD deficiencies, and one possible di-genic deficiency of a heterozygous IRF7 variant (p.Arg369Gln) and a heterozygous LOF IFIH1 variant (Δexon8). These findings suggested that up to 76 (11%) of the 682 patients tested suffered from a known form of deficiency at 8 of the 13 loci tested, and six patients suffered from a novel genetic form at 3 of the 8 loci. We finally tested whether there is an enrichment for these biochemically proven LOF genotypes in the cohort of severe COVID-19 patients. We analyzed a cohort of patients with asymptomatic SARS-CoV-2 infection and patients with mild infection, as defined by a lack of hospitalization. There was an enrichment.

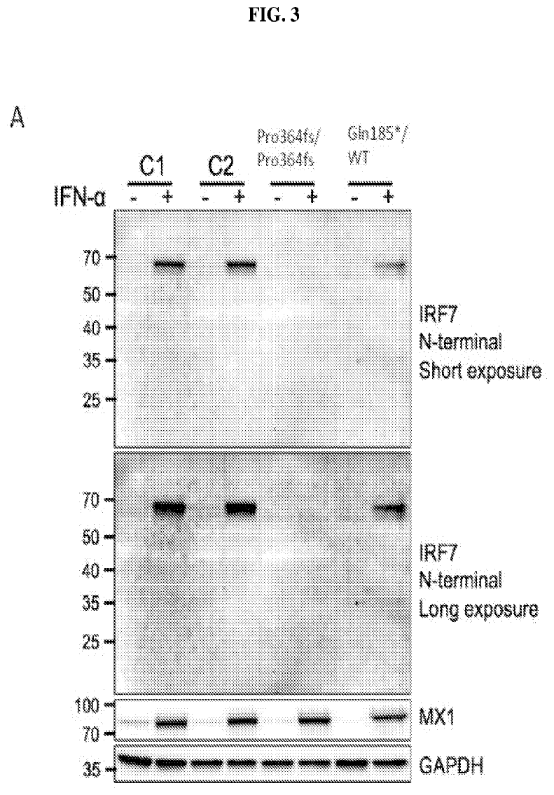

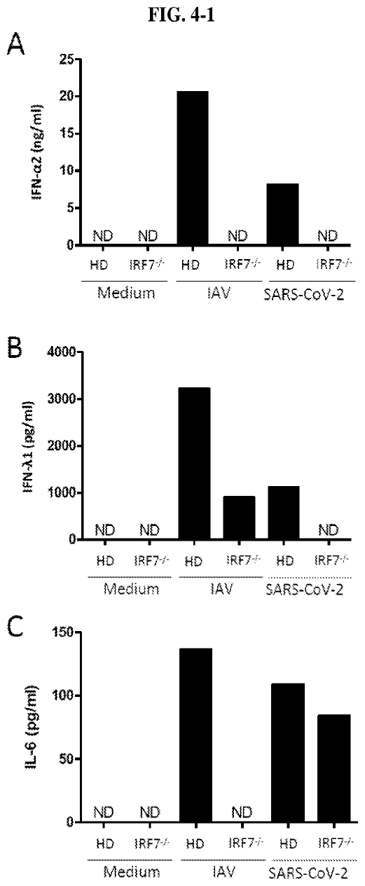

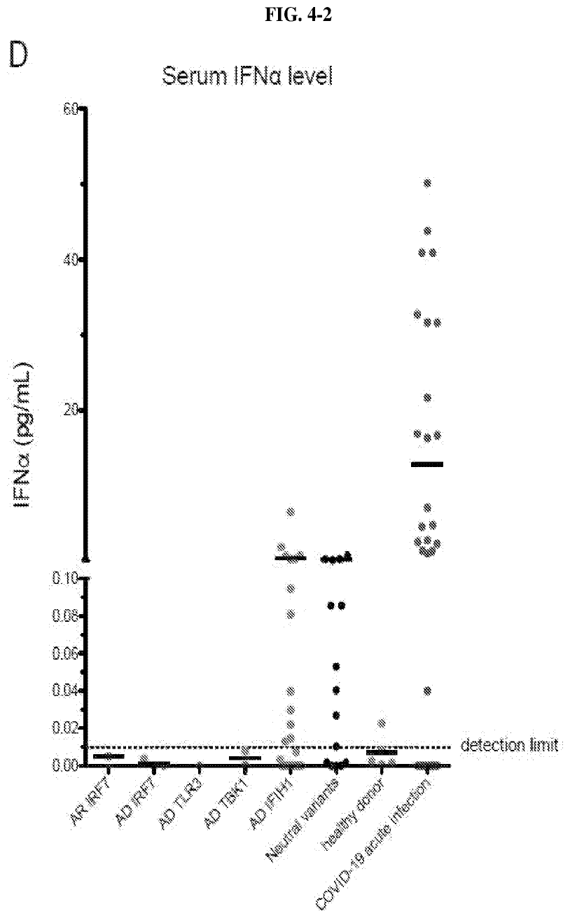

We then tested cells from patients with selected genotypes. We showed that both PHA T cell blasts from the two AR patient, one AD IRF7-deficient patient, and one heterozygous relative had decreased IRF7 expression (FIG. 3). We then tested whether some of the patients' cells are more susceptible to SARS-CoV-2 infections. We infected IRF7−/−, IRF7+/−, TLR3−/−, TLR3+/−, IFIH1−/−, IFIH1+/−SV40-transformed fibroblast cells (SV40-Fib) that stably expressed ACE2 and TMPRSS2. We found increased SARS-CoV-2 proliferation compared to SV40-Fib from healthy donors (data not shown). We then isolated pDCs from two AR and two AD IR7 deficient patients, as well as a previously reported AR IRF7 patient who suffered from influenza ARDS (20). We found that pDCs from these patients did not produce anydetectable type I or III IFNs in responding to SARS-CoV-2, despite normal IL-6 secretion, as demonstrated by ELISA (FIG. 4A-C), consistent with our findings in a previous study of the response of another IRF7-deficient patient to influenza virus (20). In summary, we showed that mutations in the type I IFN pathway lead to defective IFN production by pDCs ex vivo and increased SARS-CoV-2 proliferation in vitro. These findings showed that complete deficiencies in IFIH1, TLR3, and IRF7, resulted in both impaired production of type I IFN by pDC stimulated with SARS-CoV-2, and impaired fibroblastic-intrinsic type I IFN immunity to SARS-CoV2, thereby validating the pathogenic role of these genotypes. They further suggested that heterozygosity for LOF variations at these 3 loci, and by inference at the other 5 loci in these pathways, are also responsible for severe COVID-19.

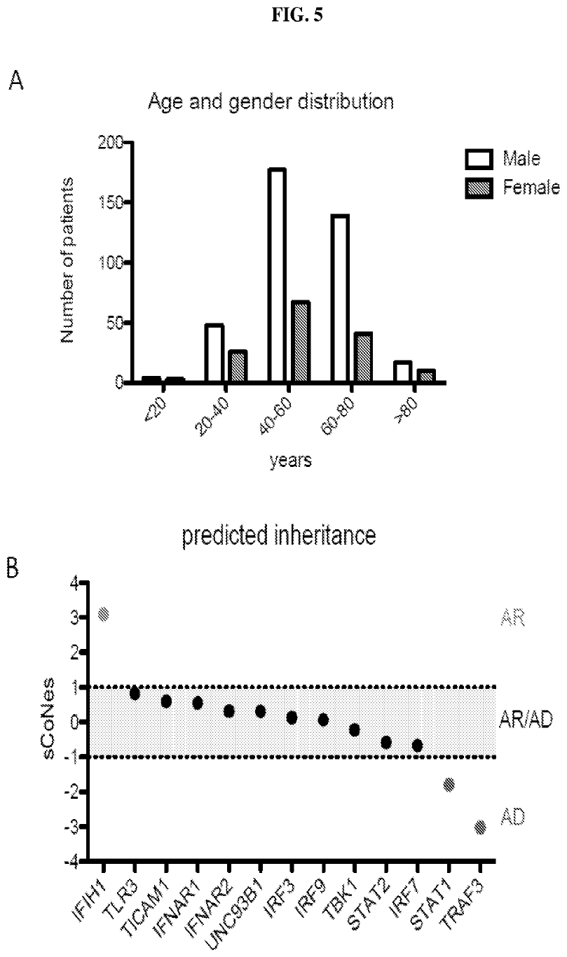

Finally, we tested whether these genotypes impair the production of type I IFN in vivo, in the course of SARS-CoV-2 infection. We measured the serum levels of the 13 IFN-α in patients during acute phase of COVID-19 infections. We found that 19 of 32 patients with samples available (1 AR IRF7, 3 AD IRF7, 1 AD TLR3, 2 AD TBK1, 25 IFIH1) had serum IFN-α levels under detection limit (FIG. 4D). The only group of patients with detectable IFN levels were heterozygous for IFIH1 LOF variations (13 of 25 patients with detectable serum IFN-α levels). This is consistent with the negative selection, which is milder on the IFIH1 locus, when compared with the other 12 loci studied (FIG. 5B). In contrast, patients with neutral mutations at loci other than IFIH1 had variable levels of IFN-α (FIG. 4D). Previously published cohorts of patients hospitalized with COVID-19 had various levels of serum IFN-α levels (34, 35). Our study includes 29 of these patients and shows that low levels of IFN-α is accounted for by mutations in type I IFN genes in some patients, with other patients displaying auto-Abs as reported in Example 2. None of these 76 patients with mutations in genes other than IFIH1 had detectable autoAb against type I IFNs (Example 2). Five of the 58 patients heterozygous for IFIH1 mutations also had such auto-Abs, and one also carried a hypomorphic IRF7 mutation. These findings indicate that the deficiencies at the 8 loci found in 76 patients have an impact in vivo in the course of SARS-CoV-2 infection, with the possible exception of AD IFIH1 deficiency, resulting in insufficient levels of type I IFN production, thereby underlying severe COVID-19.

Collectively, our data suggest that at least 76 of the 682 patients (11%) with severe COVID-19 pneumonia studied have known (7) or novel (3) genetic defects at 8 loci involved in type I IFN immunity. This discovery highlights the crucial role of type I IFN cell-intrinsic immunity in the control of SARS-CoV-2 infection in the lungs. The three pathogenic variants in IFNAR1, including two AR forms, highlights the importance of type I IFN production, when compared with that of type III IFN that is also impaired by the other defects (21, 34). This is also supported by our accompanying report of neutralizing auto-Abs against type I, but not type III, IFNs in other patients with severe COVID-19 pneumonia (Example 2). Interestingly, five of the latter 43 patients with auto-Abs carry LOF variants of IFIH1, suggesting that the two related mechanisms of disease can operate autonomously, in most cases, or synergistically, in some cases. Inborn errors of type I IFN immunity were found in patients of various ages (0.5 to 79 years) and with or without co-morbidities. These genotypes were silent until infection with SARS-CoV-2. The most thought-provoking examples are the AR deficiencies, implying that their clinical penetrance is incomplete for other viral illnesses. The two AR IRF7 deficiency was diagnosed in 49- and 50-year-old individuals with no prior history of life-threatening infections. This contrasts with the previously reported IRF7-deficient patient, who had suffered from severe influenza pneumonia at 3 years of age (20). The patients with IRF7 deficiency tested were seropositive for various influenza A and B viruses (FIG. 8, TABLE 3). These defects therefore display incomplete penetrance for influenza respiratory distress, and only manifested clinically upon infection with the more virulent SARS-CoV-2. It is even more surprising that AR IFNAR1 deficiency was diagnosed in two adult patients without history of infections prior to COVID-19. Beyond this core group of 13 genes, our findings suggest that there may be mutations of other type I IFN-related genes that cause predisposition to severe COVID-19. Our findings also suggest that early administration of type I IFN may be of therapeutic benefit in selected COVID-19 patients.

Materials and Methods

Patients

We included 682 patients hospitalized for COVID-19 in this study. Patients who were diagnosed admitted to ICU, diagnosed with pneumotitis and ARDS, or became oxygen-dependent were included. Patients developed Kawasaki-like syndrome were excluded. Their age ranged from 0.1-99, with average age 54.8 year (SD 17.1 year), and 29.4% were female. For patients enrolled in the French COVID cohort (clinicaltrials.gov NCT04262921), ethics approval was obtained from the CPP IDF VI (ID RCB: 2020-A00256-33), the IRB of ASST Ospedale San Gerardo—University of Milano-Bicocca, Monza (Italy), the IRB ofFondazione IRCCS Policlinico San Matteo, Pavia (Italy), and the Spedali Civili Ethical Committee (NP 4000—Studio CORONAlab). De-identified samples were sequenced at the NIAID undernon-human subjects research conditions; no additional IRB consent was required at the NIH.

Next Generation Sequencing

Genomic DNA was extracted from whole blood. For the 579 patients initially included, the whole exome (N=137) or whole genome (N=442) was sequenced at the Genomics Core Facility of the Imagine Institute (Paris, France), the Yale Center for Genome Analysis (USA), the New-York Genome Center (NY, USA) or at the American Genome Center (TAGC, USUHS, Bethesda, USA). For WES, libraries were generated the Twist Bioscience kits (Twist Human RefSeq Exome Kit, 36 Mb) or with the xGen Exome Research 39 Mb Panel from Integrated DNA Technologies (IDT xGen). Massively parallel sequencing was performed on a NovaSeq6000 system (Illumina).

We used the Genome Analysis Software Kit (GATK) (version 3.4-46 or 4) best-practice pipeline to analyze our WES data (36). We aligned the reads obtained with the human reference genome (hg19), using the maximum exact matches algorithm in Burrows-Wheeler Aligner (BWA)(37). PCR duplicates were removed with Picard tools (picard.sourceforge.net). The GATK base quality score recalibrator was applied to correct sequencing artifacts.

For whole genome sequencing performed on Italian cohort patients (TAGC), frozen whole blood in phlebotomy collection tubes was thawed at room temperature, and genomic DNA was extracted by an automated nucleic acid sample preparation instrument (Qiagen QIAsymphony SP) using the QIAsymphony DNA Midi Kit in 24 sample batches with a 200 uL elution volume. Genomic DNA samples were quantified using a fluorescence dye-based assay (PicoGreen dsDNA reagent) measured by a microplate reader (Molecular Devices SpectraMax Gemini XS). Genomic DNA samples were added into wells of a Covaris 96 microTUBE plate at 1,000 ng input and sheared using the Covaris LE220 Focused-ultrasonicator and settings for targeting a peak size of 410 bp (t:78; Duty:18; PIP:450; 200 cycles). Sequencing libraries are generated from fragmented DNA using the Illumina TruSeq DNA PCR-Free HT Library Preparation Kit, with minor modifications for automation (Hamilton STAR Liquid Handling System), with IDT for Illumina TruSeq DNA UD Indexes (96 indexes, 96 samples) adapters. Library size distribution and the absence of free adapters or adapter dimers were assessed by automated capillary gel-electrophoresis (Advanced Analytical Fragment Analyzer). Library concentration was determined by qPCR with the KAPA qPCR Quantification Kit (Roche Light Cycler 480 Instrument II). Sequencing libraries were normalized and combined as 24-plex pools and quantified as above, before dilution to 2.9 nM and sequencing on an Illumina NovaSeq 6000 with a S4 Reagent Kit (300 cycles) and 151+8+8+151 cycle run parameters. Primary sequencing data was demultiplexed with the Illumina HAS2.2 pipeline and sample-level quality control was performed for base quality, coverage, duplicates and contamination (FREEMIX <0.05 by VerifyBamID) was conducted.

CNV Detection

We searched for deletions in the 13 genes of interest from the NGS data using both HMZDelFinder (38) and CANOES (39) algorithms.

DNA Cloning and Mutagenesis of IFIH1 Variants

For IFIH1 testing, missense variants in IFIH1 were selected based on Combined Annotation Dependent Depletion (CADD) score (>20) and MAF in gnomAD (>1×10₋₅). Variants were introduced into the WT MDA5 construct via site-directed mutagenesis, using primer-directed linear amplification with CloneAmp HiFi PCR Premix (Takara Bio), followed by DpnI digestion of methylated template DNA (New England Biolabs). All inserted variants were confirmed by Sanger dideoxy sequencing of plasmids purified from transformed competent E. Coli (TOP10/DHSalpha) using mini-prep kits (Zymo Research). pIFNB-GL3 firefly luciferase and pRL-TK Renilla luciferase reporter plasmids were kindly provided by Y. He (NIDDK, NIH).

IRF7 and TBK1 IFN-Beta Reporter Assays

IRF7 and TBK1 luciferase assays were performed as following: wildtype HEK293T cells (IRF7 and TBK1) or IRF3-deficient HEK293T cells (IRF3) were co-transfected with a mixture of the IFN-β firefly luciferase reporter plasmid, the pRL-TK-Renilla-luciferase plasmid, and any appropriate additional constructs for 24 hours. Empty vector was added to ensure the presence of the same total amounts of DNA in the assay. For the IRF7 and IRF3 luciferase assay, cells were infected with Sendai virus (20 HAU/well) for 24 hours. For TBK1, cells were left unstimulated. Luciferase activity was measured with the Dual-Luciferase Reporter Assay system (Promega) according to the manufacturer's instructions. Data were normalized for transfection efficiency by dividing firefly luciferase activity by that of Renilla luciferase activity.

IFIH1 Transfections and Luciferase Assays

293T-RU cells (a subclone of 293T cells adapted for transfection in suspension) were maintained in complete DMEM (Dulbecco's modified Eagle medium (DMEM) (Gibco/Thermo Fisher Scientific), 2 mM L-glutamine plus 10% fetal bovine serum (FBS; Gibco or Sigma), 100 U/ml penicillin, 100 μg/ml streptomycin (Gibco)). Cells were passaged every two days, after washing with trypsin-EDTA (Gibco) in PBS. A 7× master mix containing polyethylenimine (PEI, 1 μg), pIFNB-GL3 (200 ng), pRL-TK (20 ng), and 60 μl serum-free DMEM per well (7 wells=420 l) was added to 1.5 mL tubes containing 350 ng WT or mutant MDA5 plasmid. The tubes were vortexed and incubated at room temperature for 15 minutes. The 293T-RU cells were released by trypsin treatment and resuspended at a density of 1.33×106 cells/ml in DMEM+20% FBS; an aliquot of 420 μL was dispensed into each 1.5 ml tube containing PEI/DNA mix. We used 120 μL (8×104 cells/well) of each variant mixture to seed each of four wells of a 96-well flat-bottomed culture plate. The remaining cells were plated in a 48-well plate for the production of lysates (see immunoblotting below). At 24 h post-transfection, duplicate wells were left unstimulated or were stimulated with 2 μg/mL poly I:C, as follows: 0.4 μg poly I:C (in 25 μl OptiMEM) was incubated with +1 μl Lipofectamine 2000 (Thermo Fisher Scientific; in 25 μL OptiMEM) at room temperature for 20 minutes. We then added 150 μL of DMEM+10% FBS. The total volume (200 l/well) was added to each well of the 96-well plate after the previous cell culture medium had been carefully removed by aspiration, and cells were incubated for an additional 20 hours. After stimulation, cells were washed once in PBS and lysed in 200 μL 1× passive lysis buffer (Promega). Dual luciferase assays were performed in accordance with the kit manufacturer's protocol (Promega), on microplate readers (BioTek Synergy H1 or BMG Labtech Fluostar Omega), with 20 μL of each cell lysate in white-walled 96 well plates. The percentage activity relative to WT MDA5 was calculated by determining the normalized firefly:Renilla luciferase ratio for each variant divided by the normalized WT value with and without poly I:C stimulation, and plotted with Prism 8 software (GraphPad). For immunoblotting, transfected cells from the 48-well plate were washed in PBS and lysed in 25 μL NP40 buffer for 1 hour on ice. Following centrifugation to clear insoluble material (10 min at 14,000 rpm), lysates were mixed with 2× Laemmli SDS sample buffer+5 2-ME and 15 μL samples were loaded onto 4-20% Tris-glycine gels (Bio-Rad) and separated by SDS-PAGE. The bands obtained were transferred to nitrocellulose membranes (TransBlot Turbo, Bio-Rad), which were blocked by incubation for one hour in Odyssey blocking buffer (LiCOR Biosciences). Membranes were incubated for 16 hours with anti-MDA5 (clone D74E4, Cell Signaling Technology), anti-HSP90 (clone 68, BD Biosciences) or anti-β-actin (clone AC-15, Sigma) antibodies. The membrane was washed with TBS-Tween 20, and the signal was detected with appropriate HRP- or IRDye-conjugated secondary Abs and visualized by enhanced chemiluminescence (ECL, Pierce) or on an Odyssey Fc Imaging System (LiCOR).

TLR3 Functional Test

The TLR3-deficient P2.1 fibrosarcoma cell line was kindly provided by Douglas W. Leaman. Stably transfected P2.1 cells were established by transfecting with pUNO-TLR3 (Invivogen, USA) in the presence of X-tremegene 9, with selection by blasticidin treatment (10 μg/ml). P2.1 cells were maintained in Dulbecco's modified Eagle medium (DMEM) supplemented with 10% fetal calf serum (FCS). The cells were stimulated with TLR3 agonist poly(I:C) (Amersham) at a concentration of 25 pg/mL. Four hours after stimulation, cells were harvested, and their cytokine mRNA production was analyzed by quantitative RT-PCR, as previously described (19).

Single-Molecule Array (Simoa) IFNα Digital ELISA

Serum IFN-α concentrations were determined with Simoa technology, with reagents and procedures obtained from Quanterix Corporation (Quanterix Simoa™ IFNα Reagent Kit, Lexington, Mass., USA). According to the manufacturer's instructions, the working dilutions were 1:2 for all sera, in working volumes of 170 μL. In the first step of the assay, capture beads coated with an anti-cytokine Ab were combined with the serum. After washing, the biotinylated detector Ab was added to the reaction to bind the captured cytokine. Following a second wash, streptavidin-p-galactosidase (SBG) was added to bind the detector Ab, resulting in enzyme labeling of the captured cytokine. After washing, the beads were resuspended in a resorufin β-D-galactopyranoside (RGP) substrate solution and immediately transferred to a Simoa disc array (Quanterix Simoa™ Disc Kit) for individual capture in the microwells. The β-galactosidase on the captured cytokine hydrolyzed the RGP substrate, yielding a fluorescent signal, for IFNα determination. At low cytokine concentrations, the percentage of bead-containing wells in the array displaying a positive signal is proportional to the amount of cytokine present in the sample (digital measurement). At higher concentrations, when most of the bead-containing wells have one or more labeled cytokine molecules, the total fluorescence signal is proportional to the amount of cytokine present in the sample (analog measurement). Cytokine concentrations in serum samples were interpolated from standard curves. The lower limit of detection was 5 fg/mL. The upper limit of quantification was 52,200 fg/mL (i.e., above this concentration, we did not dilute the sample further).

pDC Purification

Peripheral blood mononuclear cells (PBMCs) were isolated by Ficoll density gradient centrifugation (Ficoll-Paque; GE Healthcare). PDCs were sorted magnetically with the Human Plasmacytoid DC Enrichment Kit (StemCell), according to the manufacturer's instructions. For the assessment of pDC enrichment, cells were stained with zombie violet fixable viability stain (Biolegend), FITC anti-CD16 (BD, clone NKP15), FITC anti-CD14 (Miltenyi Biotec, clone TUK4), FITC anti-CD19 (Miltenyi Biotec, clone LT19), FITC anti-CD20 (BD, clone 2H7), FITC anti-CD56 (Biolegend, clone HCD56), FITC anti-CD3 (BD, clone HIT3a), BV650 anti-CD4 (Biolegend, clone OKT4), APC-Vio770 anti-CD2 (Miltenyi Biotec, clone LT2), APC anti-CD5 (BD, clone UCHT2), and BV785 anti-CD123 (Biolegend, clone 6H6) antibodies. PDCs were gated as Live, Lin− (CD16, CD14, CD19, CD20, CD56 and CD3), CD2−CD5−, and CD4+CD123+ cells. Acquisitions were performed on a LSRFortessa machine (BD Biosciences). Data were analyzed with FlowJo software (TreeStar).

pDC Activation by SARS-CoV-2 and Cytokine Production

pDCs from an IRF7−/− patient and a healthy donor matched for age and sex were cultured at a density of 3×10⁵ and 5×10⁵ cells/mL, respectively, for 12 h, in the presence of medium alone (RPMI 1640 Medium with GlutaMAX, 10% FBS, 1% MEM NEAA, 1% sodium pyruvate, and 1% penicillin/streptomycin), influenza virus (Charles River, A/PR/8/34, 2 pg/mL), or the SARS-CoV-2 primary strain 22095 (GISAID accession ID: EPI_ISL_469284) at a multiplicity of infection (MOI) of 2. This virus was isolated from nasopharyngeal swabs and amplified in Vero cells. After 12 hours of culture, pDC supernatant was collected forcytokine quantification. Supernatants were incubated for 2 hours at room temperature with 0.4% (v/v) Triton X-100 before quantification, to inactivate the infectious particles of SARS-CoV-2. IFN-α2, and IL-6 levels were measured in BD cytometric bead arrays (CBAs), in accordance with the manufacturer's protocol, with a 20 pg/mL detection limit. Acquisitions were performed on a LSRFortessa machine (BD Biosciences), and cytokine concentration was determined with FCAP Array Software (BD Biosciences). IFN-λ1 secretion was measured by enzyme-linked immunosorbent assay (ELISA) (R&D Systems, DuoSet DY7246), in accordance with the manufacturer's instructions. The optical density (OD) of the supernatant was defined as its absolute OD value, minus the OD for the blank wells. The detection limit was 85 pg/mL and all samples were run in duplicate. Absorbance was measured on a CLARIOstar Plus microplate reader (BMG Labtech).

Influenza Virus Protein

Influenza virus protein microarrays (IVPM) were produced by spotting recombinant influenza virus hemagglutinins (HAs) onto epoxysilane-coated glass slides (Schott, Mainz, Germany), as previously described (42). Each microarray slide contained 24 identical arrays consisting of 13 HAs diluted in 0.1% milk in PBS, printed in triplicate, at a volume of 30 nL per spot, at a concentration of 100 μg/mL. Three different array designs were included in this study. IVPMs were vacuum-packed and stored at −80° C. until use. Before use, IVPM slides were warmed to room temperature. They were then incubated in a chamber maintained at 95-98% relative humidity for 2 hours, to bind proteins to the slide and inactivate epoxysilane residues not in contact with recombinant HAs. The IVPM slides were then inserted into 96-well microarray gaskets (Arrayit, Sunnyvale, Calif., USA), dividing each slide into 24 separate arrays. These arrays were blocked with 220 μL 3% milk in PBS supplemented with 0.1% Tween 20 (PBS-T) for 2 hours. The blocking solution was then removed from the arrays, and serum samples diluted 1:100 in 1% milk in PBS-T were incubated with the arrays at a volume of 100 μL and diluted 1:10 across three arrays. IVPM arrays were washed three times with 220 μL PBS-T, and then 50 μL of Cy5-labeled anti-human IgG secondary antibody diluted 1:3000 in 1% milk in PBS-T was added to each array and the array was incubated for one hour. The secondary antibody solution was removed, each array was washed three times with PBS-T and the slides were removed from their gaskets for rinsing with PBS-T and deionized water. They werethen dried with an air compressor. Arrays were imaged with a Vidia microarray scanner (Indevr, Boulder, Colo., USA), using an exposure time of 1000 ms. Area under the curve was calculated from the median spot fluorescence, taking the total peak area with a minimum threshold of 0.04.

Western Blot IRF7

PBMCs from two controls, P1 (Q364fs) and P2 (Q185*), were activated with phytohemaglutinin (PHA; Sigma) and IL-2 (10 ng/mL; Thermo Fisher Scientific) in RPMI supplemented with 10% FCS. IL-2 was renewed every two days. After 14 days, expanded T lymphocytes were harvested, counted and starved by overnight incubation of 5×10₆ cells/mL in serum-free X-VIVO 20 medium (Lonza) in the presence or absence of IFN-α (105 IU; MSD). Cells were collected, washed in 1×PBS and lysed in RIPA buffer supplemented with Pierce Universal Nuclease for Cell Lysis (1/100, Thermo Fisher Scientific) and a protease inhibitor cocktail: aprotinin (Sigma, 10 g/mL), PMSF (Sigma, 1 mM), leupeptin (Sigma, 10 μg/mL). The proteins were separated by SDS-PAGE and immunoblotting was performed with Abs against IRF7 (G-8, Santa Cruz), MX1 (rabbit polyclonal, Proteintech) or GAPDH (FL335, Santa Cruz).

Specific aspects and information regarding the variants identified in the COVID-19 patients cohort are provided below in Table 1 and Table 2. Human protein encoding sequencs and amino acid sequences for the various referenced type I interferon (IFN) pathway proteins are known and available. For example, protein sequences in public databases are known and include the following: IRF7 (NP_004022.2), TLR3 (NP_003256.1), TBK1 (NP_037386.1, NM_013254.4), IRF3 (AAH09395), TICAM1 (NP_891549), IFNAR1 (NP_000620.2), UNC93B1 (NP_112192.2), IFIH1 (AAI11751.1, NP_071451.2), IFNAR2 (NP_001276054.1), IRF9 (NP_001372329.1), STAT1 (NP_009330.1), STAT2 (NP-005410.1) and TRAF3 (NP_003291.2).

TABLE 1
Deleterious Variants Identified in COVID-19 Patients

| | | Novel | | | | | |
|---|---|---|---|---|---|---|---|
| | Inher- | Genetic | | | | | |
| Gene | itance | Form | Genotype | Functional test | Age | Gender | Severity |
| | AR | | p.Pro364ts/p.Pro364ts | hypomorphic | 49 | F | ARDS, ECMO |
| IRF7 | AR | | p.Met371Val/p.Asp117Asn | LOF/hypomorphic | 50 | M | Severe, oxygen-dependent |
| IRF7 | AD | Novel | p.Gln185* | LOF | 44 | M | ARDS, ECMO |
| IRF7 | AD | Novel | p.Pro246fs | hypomorphic | 41 | M | Severe, oxygen-dependent |
| IRF7 | AD | Novel | p.Arg7fs | LOF | 60 | M | Critical |
| IRF7 | AD | Novel | p.Arg369Gln | hypomorphic | 69 | M | Critical |
| TLR3 | AD | | p.Pro554Ser | LOF | 68 | M | Critical |
| TLR3 | AD | | p.Ser339fs | LOF | 40 | M | ARDS, TCU |
| TLR3 | AD | | p.Irp769** | LOF | 77 | M | Oxygen-dependent |
| TBK1 | AD | | p.Arg308* | LOF | 17 | M | ICU, oxygeno-dependent |
| TBK1 | AD | | p.Phe24Ser | LOF | 46 | F | Severe, oxygeno-dependent |
| TBK1 | AD | | p.Arg384Gln | hypomorphic | 43 | F | ARDS |
| IRF3 | AD | | p.Asn146Lys | hypomorphic | 60 | F | Critical |
| IRF3 | AD | | p.Glu49del | hypomorphic | 23 | F | Severe, oxygen-dependent |
| TICAM1 | AD | | p.Ser60Cys | LOF | 61 | F | TCU, oxygen-dependent |
| IFNAR1 | AR | | p.Trp73Cys | LOF | 38 | M | ARDS |
| IFNAR1 | AR | | p.Ser422Arg | LOF | 26 | M | ARDS |
| IFNAR1 | AD | Novel | p.Pro335del | LOF | 23 | F | Critical |
| UNC93B1 | AD | Novel | p.Glu96* | LOF | 48 | M | Severe, oxygen-dependent |
| IFIH1 | AD | | Δexon14 | LOF | 72 | M | Critical |
| IFIH1 | AD | | Δexon14 | LOF | 57 | M | ARDS, ICU, Septic shock, Renal failure |
| IFIH1 | AD | | Δexon14 | LOF | 71 | M | Critical |
| IFIH1 | AD | | Δexon14 | LOF | 52 | M | Critical |
| IFIH1 | AD | | Δexon14 | LOF | 44 | M | ARDS |
| IFIH1 | AD | | Δexon14 | LOF | 32 | F | Severe, oxygen-dependent |
| IFIH1 | AD | | Δexon14 | LOF | 58 | M | ARDS |
| IFIH1 | AD | | Δexon14 | LOF | 52 | M | ARDS, ECMO |
| IFIH1 | AD | | Δexon14 | LOF | 67 | M | severe |
| IFIH1 | AD | | Δexon14 | LOF | 65 | F | Critical |
| IFIH1 | AD | | Δexon14 | LOF | 68 | M | Critical |
| IFIH1 | AD | | Δexon14 | LOF | 73 | M | Severe |
| IFIH1 | AD | | Δexon14 | LOF | 59 | F | Pneumonitis |
| IFIH1 | AD | | Δexon8 | LOF | 9 | M | ARDS, cardiomyopathy, multiorganic failure |
| IFIH1 | AD | | Δexon8 | LOF | 50 | M | Severe, oxygen-dependent |
| IFIH1 | AD | | Δexon8 | LOF | 63 | M | Critical |
| IFIH1 | AD | | Δexon8 | LOF | 67 | F | Critical |
| IFIH1 | AD | | Δexon8 | LOF | 74 | M | Critical |
| IFIH1 | AD | | Δexon8 | LOF | 70 | M | Critical |
| IFIH1 | AD | | Δexon8 | LOF | 59 | M | Critical |
| IFIH1 | AD | | Δexon8 | LOF | 61 | F | Critical |
| IFIH1 | AD | | Δexon8 | LOF | 55 | M | Critical |
| IFIH1 | AD | | Δexon8 | LOF | 69 | M | Critical |
| IFIH1 | AD | | Δexon8 | LOF | 59 | F | Severe |
| IFIH1 | AD | | Δexon8 | LOF | 46 | M | ARDS, ECMO |
| IFIH1 | AD | | Δexon8 | LOF | 49 | M | Severe, oxygen-dependent |
| | | | | | | | macrophage activation syndrome |
| IFIH1 | AD | | Δexon8 | LOF | 49 | M | Severe |
| IFIH1 | AD | | Δexon8 | LOF | 25 | M | Severe, oxygen-dependent |
| IFIH1 | AD | | Δexon8 | LOF | 41 | M | Pneumonitis |
| IFIH1 | AD | | Δexon8 | LOF | 39 | M | ARDS |
| IFIH1 | AD | | Δexon8 | LOF | 48 | F | ARDS, |
| | | | | | | | oxygeno-dependent |
| IFIH1 | AD | | p.Ala589fs | LOF | 23 | M | ARDS, ECMO |
| IFIH1 | AD | | p.Ala589fs | LOF | 40 | M | ARDS |
| IFIH1 | AD | | p.Ala589fs | LOF | 31 | F | Pneumonitis |
| IFIH1 | AD | | p.Cys565Phe | LOF | 63 | M | Critical |
| IFIH1 | AD | | p.Glu627* | LOF | 49 | F | Critical |
| IFIH1 | AD | | p.Glu627* | LOF | 57 | F | Severe |
| IFIH1 | AD | | p.Glu627* | LOF | 68 | M | Critical |
| IFIH1 | AD | | p.Glu627* | LOF | 47 | M | ARDS |
| IFIH1 | AD | | p.Ile923Val | LOF | 55 | M | Critical, intubated |
| IFIH1 | AD | | p.Ile923Val | LOF | 79 | F | Severe |
| IFIH1 | AD | | p.Ile923Val | LOF | 45 | M | Severe, oxygen-dependent |
| IFIH1 | AD | | p.Ile923Val | LOF | 66 | M | Critical |
| IFIH1 | AD | | p.Ile923Val | LOF | 38 | M | ARDS, macrophage activation |
| | | | | | | | syndrome, thromboembolic event |
| IFIH1 | AD | | p.Ile923Val | LOF | 46 | F | Critical |
| IFIH1 | AD | | p.Ile923Val | LOF | 65 | M | Critical |
| IFIH1 | AD | | p.Ile923Val | LOF | 68 | M | severe, ICU |
| IFIH1 | AD | | p.Leu679fs | LOF | 39 | M | Critical |
| IFIH1 | AD | | p.Lys365Glu | LOF | 62 | F | Severe, oxygen-dependent |
| IFIH1 | AD | | p.Phe744Leu | LOF | 44 | M | Severe, oxygen-dependent |
| IFIH1 | AD | | p.Pro866Leu | LOF | 63 | M | Critical |
| IFIH1 | AD | | p.Val929Ile | LOF | 48 | F | Pneumonitis |
| IFIH1 | AD | | p.Arg728* | LOF | 60 | M | Severe, oxygen-dependent |
| IFIH1 | AD | | p.Thr767fs | LOF | 36 | F | Severe, oxygen-dependent |
| IFIH1 | AD | | p.Tyr25* | LOF | 35 | M | ICU, no intubation |
| IFIH1 | AD | | p.Val296fs | LOF | <1 | F | ARDS |
| IFIH1 | AD | | p.Cys910Ser | LOF | 68 | M | ARDS |
| IFIH1 | AD | | p.Met570Thr | hypomorphic | 64 | F | ARDS |

TABLE 2
Candidate Variants found in Severe COVID-19 Patients

| | | | | Functional | Number |
|---|---|---|---|---|---|
| Gene | Genotype | Zygoucity | pLOF | test | of patients |
| IFIH1 | Δexon14 | het | Y | LOF | 13 |
| IFIH1 | Δexon8 | het | Y | LOF | 18 |
| IFIH1 | p.Ala319Thr | het | | neutral | 1 |
| IFIH1 | p.Ala589fs | het | Y | LOF | 3 |
| IFIH1 | p.Ala788Thr | het | | neutral | 1 |
| IFIH1 | p.Arg386His | het | | neutral | 1 |
| IFIH1 | p.Arg598His | het | | neutral | 2 |
| IFIH1 | p.Arg728* | het | Y | LOF | 1 |
| IFIH1 | p.Cys565Phe | het | | LOF | 1 |
| IFIH1 | p.Cys910Ser | het | | LOF | 1 |
| IFIH1 | p.Gln378His | het | | neutral | 1 |
| IFIH1 | p.Gln955Glu | het | | neutral | 2 |
| IFIH1 | p.Gln966His | het | | neutral | 1 |
| IFIH1 | p.Glu627** | het | Y | LOF | 4 |
| IFIH1 | p.Glu937Lys | het | | neutral | 1 |
| IFIH1 | p.Ile146Val | het | | neutral | 1 |
| IFIH1 | p.Ile923Val | het | | LOF | 8 |
| IFIH1 | p.Ile940Val | het | | neutral | 1 |
| IFIH1 | p.Leu679fs | het | Y | LOF | 1 |
| IFIH1 | p.Lys349Arg | het | | neutral | 3 |
| IFIH1 | p.Lys365Glu | het | | LOF | 1 |
| IFIH1 | p.Lys949Asn | het | | neutral | 1 |
| IFIH1 | p.Met558Val | het | | neutral | 1 |
| IFIH1 | p.Met570Thr | het | | hypomorphic | 1 |
| IFIH1 | p.Met95Val | het | | neutral | 2 |
| IFIH1 | p.Phe744Leu | het | | LOF | 1 |
| IFIH1 | p.Pro218Leu | het | | neutral | 1 |
| IFIH1 | p.Pro480His | het | | neutral | 1 |
| IFIH1 | p.Pro866Leu | het | | LOF | 1 |
| IFIH1 | p.Q966H | het | | neutral | 1 |
| IFIH1 | p.Ser645Gly | het | | neutral | 1 |
| IFIH1 | p.Thr702Ile | het | | neutral | 7 |
| IFIH1 | p.Thr767fs | het | Y | LOF | 1 |
| IFIH1 | p.Tyr25 ** | het | Y | LOF | 1 |
| IFIH1 | p.Val296fs | het | Y | LOF | 1 |
| IFIH1 | p.Val929Ile | het | | LOF | 1 |
| IFNAR1 | p.Ala24Val | het | | neutral | 2 |
| IFNAR1 | p.Ala424Thr | het | | neutral | 1 |
| IFNAR1 | p.Asn88Ser | het | | neutral | 1 |
| IFNAR1 | p.Gln80His | het | | neutral | 1 |
| IFNAR1 | p.Glu386Lys | het | | neutral | 1 |
| IFNAR1 | p.Glu515Lys | het | | neutral | 1 |
| IFNAR1 | p.Ile169Met | het | | neutral | 1 |
| IFNAR1 | p.Ile183Val | het | | neutral | 1 |
| IFNAR1 | p.Ile508Thr | het | | neutral | 1 |
| IFNAR1 | p.Pro335del | het | | LOF | 1 |
| IFNAR1 | p.Ser422Arg | hom | | LOF | 1 |
| IFNAR1 | p.Thr83Ala | het | | neutral | 1 |
| IFNAR1 | p.Trp73Cys | hom | | LOF | 1 |
| IFNAR2 | p.Asn146Ser | het | | neutral | 1 |
| IFNAR2 | p.Glu138_Phe139insSer | het | | neutral | 1 |
| IFNAR2 | p.Glu138Val | het | | neutral | 1 |
| IFNAR2 | p.Glu37Gln | het | | neutral | 2 |
| IFNAR2 | p.His283Arg | het | | neutral | 1 |
| IFNAR2 | p.Ile197Thr | het | | neutral | 1 |
| IFNAR2 | p.Met73Val | het | | neutral | 1 |
| IFNAR2 | p.Pro295Leu | het | | neutral | 1 |
| IFNAR2 | p.Pro346Ser | het | | neutral | 1 |
| IFNAR2 | p.Pro362Ser | het | | neutral | 1 |
| IFNAR2 | p.Ser215Gly | het | | neutral | 1 |
| IFNAR2 | p.Ser324Asn | het | | neutral | 1 |
| IFNAR2 | p.Tyr318Cys | het | | neutral | 1 |
| IRF3 | cryptic splicing | het | Y | normal splicing/ | 1 |
| | site | | | neutral | |
| IRF3 | p.Arg227Gln | het | | neutral | 1 |
| IRF3 | p.Arg342Gln | het | | neutral | 1 |
| IRF3 | p.Asn146Lys | het | | hypomorphic | 1 |
| IRF3 | p.Glu49del | het | | hypomorphic | 1 |
| IRF3 | p.Thr406Ser | het | | neutral | 1 |
| IRF7 | p.Arg7fs | het | Y | LOF | 1 |
| IRF7 | p.Ala419Thr | het | | neutral | 1 |
| IRF7 | p.Arg131Gln | het | | neutral | 4 |
| IRF7 | p.Arg369Gln | het | | hypomorphic | 1 |
| IRF7 | p.Arg423Pro | het | | neutral | 1 |
| IRF7 | p.Arg37His | het | | neutral | 1 |
| IRF7 | p.Asp117Asn | compound het | | hypomorphic | 1 |
| IRF7 | p.Asp204Asn | het | | neutral | 6 |
| IRF7 | p.Gln185* | het | Y | LOF | 1 |
| IRF7 | p.Glu71Ala | het | | neutral | 1 |
| IRF7 | p.Gly146Arg | het | | neutral | 1 |
| IRF7 | p.Gly214Glu | het | | neutral | 1 |
| IRF7 | p.Gly247Arg | het | | neutral | 4 |
| IRF7 | p.His163Leu | het | | neutral | 1 |
| IRF7 | p.Met371Val | compound het | | LOF | 1 |
| IRF7 | p.Phe95Ser | het | | neutral | 1 |
| IRF7 | p.Pro246fs | het | Y | hypomorphic | 1 |
| IRF7 | p.Pro364fs | hom | Y | hypomorphic | 1 |
| IRF7 | p.Thr254Ala | het | | neutral | 1 |
| IRF9 | p.Glu209Gly | het | | neutral | 1 |
| IRF9 | p.Gly203Arg | het | | neutral | 1 |
| IRF9 | p.Thr153lle | het | | neutral | 1 |
| IRF9 | p.Val230Met | het | | neutral | 1 |
| STAT1 | p.Gln595His | het | | neutral | 1 |
| STAT1 | p.Ser485Thr | het | | neutral | 1 |
| STAT2 | p.Ala824Gly | het | | neutral | 1 |
| STAT2 | p.Arg587His | het | | neutral | 1 |
| STAT2 | p.Asp449Asn | het | | neutral | 1 |
| STAT2 | p.Gln289Pro | het | | neutral | 1 |
| STAT2 | p.Gln309Arg | het | | neutral | 1 |
| STAT2 | p.Gln84Lys | het | | neutral | 1 |
| STAT2 | p.Glu157Asp | het | | neutral | 1 |
| STAT2 | p.Leu232Met | het | | neutral | 1 |
| STAT2 | p.Leu791Trp | het | | neutral | 1 |
| STAT2 | p.Pro489Arg | het | | neutral | 1 |
| STAT2 | p.Pro489His | het | | neutral | 1 |
| STAT2 | p.Pro489Leu | het | | neutral | 1 |
| STAT2 | p.Pro731Ser | het | | neutral | 1 |
| STAT2 | p.Val342Ile | het | | neutral | 1 |
| STAT2 | p.Val37Phe | net | | neutral | 1 |
| TBK1 | p.Ala535Thr | het | | neutral | 1 |
| TBK1 | p.Arg308* | het | Y | LOF | 1 |
| TBK1 | p.Arg384Gln | het | | hypomorphic | 1 |
| TBK1 | p.Asn388Ser | het | | neutral | 1 |
| TBK1 | p.Glu653Gln | het | | neutral | 1 |
| TBK1 | p.Ile397Thr | het | | neutral | 2 |
| TBK1 | p.Ile522Met | het | | neutral | 1 |
| TBK1 | p.Leu508Ile | het | | neutral | 1 |
| TBK1 | p.Met184Val | het | | neutral | 1 |
| TBK1 | p.Phe24Ser | het | | LOF | 1 |
| TBK1 | p.Pro659Ser | het | | neutral | 1 |
| TBK1 | p.Thr331Ile | het | | neutral | 1 |
| TBK1 | p.Val152Leu | het | | neutral | 1 |
| TICAM1 | p.Ala111Thr | het | | neutral | 1 |
| TICAM1 | p.Arg195Gln | het | | neutral | 1 |
| TICAM1 | p.Arg71Gln | het | | neutral | 1 |
| TICAM1 | p.Asp557Asn | het | | neutral | 1 |
| TICAM1 | p.Gln392Lys | het | | neutral | 1 |
| TICAM1 | p.Gln702Arg | het | | neutral | 1 |
| TICAM1 | p.Gly598Trp | het | | neutral | 1 |
| TICAM1 | p.Leu162Val | het | | neutral | 1 |
| TICAM1 | p.Leu386Pro | het | | neutral | 1 |
| TICAM1 | p.Met595Leu | het | | neutral | 1 |
| TICAM1 | p.Pro620Ser | het | | neutral | 1 |
| TICAM1 | p.Ser465Asn | het | | neutral | 1 |
| TICAM1 | p.Ser60Cys | het | | LOF | 1 |
| TICAM1 | p.Thr157Met | het | | neutral | 1 |
| TICAM1 | p.Thr377Ile | het | | neutral | 1 |
| TICAM1 | p.Thr4Ile | het | | neutral | 1 |
| TICAM1 | p.Val302Leu | het | | neutral | 1 |
| TICAM1 | p.Val80Met | het | | neutral | 1 |
| TLR3 | p.Arg251Gly | het | | neutral | 1 |
| TLR3 | p.His891Arg | het | | neutral | 1 |
| TLR3 | p.Phe732Leu | het | | neutral | 1 |
| TLR3 | p.Pro554Ser | het | | LOF | 1 |
| TLR3 | p.Ser339fs | het | Y | LOF | 1 |
| TLR3 | p.Ser810Gly | het | | neutral | 1 |
| TLR3 | p.Trp769* | het | Y | LOF | 1 |
| TRAF3 | p.Ala188Val | het | | neutral | 1 |
| TRAF3 | p.Ala412Thr | het | | neutral | 1 |
| TRAF3 | p.Arg321Gln | het | | neutral | 1 |
| TRAF3 | p.Asn245Lys | het | | neutral | 1 |
| TRAF3 | p.Lys160Asn | net | | neutral | 1 |
| TRAF3 | p.Trp266Cys | het | | neutral | 1 |
| TRAF3 | p.Val240Ile | het | | neutral | 1 |
| TRAF3 | p.Val367Met | het | | neutral | 1 |
| UNC93B1 | p.Ala361Thr | het | | neutral | 2 |
| UNC93B1 | p.Ala385Thr | het | | neutral | 1 |
| UNC93B1 | p.Glu96 | het | Y | LOF | 1 |
| UNC93B1 | p.Gly592Arg | het | | neutral | 2 |
| UNC93B1 | p.Ser164Leu | het | | neutral | 1 |
| UNC93B1 | p.Tyr435Cys | het | | neutral | 1 |
| UNC93B1 | p.Val379Met | het | | neutral | 1 |
| UNC93B1 | p.Val402Leu | het | | neutral | 1 |

TABLE 3
HA Abbreviation Key

| | |
|---|---|
| PR34 H1 | A/Puerto Rico/8/34 (H1N1) |
| TX91 H1 | A/Texas/36/91 (H1N1) |
| NC99 H1 | A/New Caledonia/20/1999 (H1) |
| Cal09 H1 | A/California/04/2009 (H1N1) |
| Mich15 H1 | A/Michigan/45/2015 (H1) |
| Nic13 H1 | A/Nicaragua/1815_01_TR2/2013 H1 |
| Bris 18 H1 | A/Brisbane/02/2018 (H1) |
| H2 | A/Japan/305/57 (H2N2) |
| H5 | A/Indonesia/05/2005 (H5) |
| H6 | A/mallard/Sweden/81/02 (H6N4) |
| H11 | A/shoveler/Netherlands/18/99 (H11N9) |
| H16 | A/black headed gull/Sweden/5/99 (H16N3) |
| H13 | A/black headed gull/Sweden/1/1999 (H13) |
| H8 | A/mallard/Sweden/24/2002 (H8) |
| H12 | A/mallard/Interior Alaska/7MP0167/2007 (H12) |
| H9 | A/guinea fowl/Hong Kong/WF10/1999 (H9) |
| HK68 H3 | A/Hong Kong/1/1968 (H3) |
| Pan99 H3 | A/Panama/2007/1999 (H3) |
| Wisc05 H3 | A/Wisconsin/67/2005 (H3) |
| Wyo03 H3 | A/Wyoming/3/2003 (H3) |
| Switz13 H3 | A/Switzerland/9715293/2013 (H3N2) |
| Sing16 H3 2016 | A/Singapore/INFIMH-16-0019/2016 (H3) |
| Kan17 H3 | A/Kansas/14/2017 (H3) |
| H4 | A/red knot/Delaware/541/1988 (H4) |
| H14 | A/mallard/Gurjev/263/82 (H14N5) |
| H10 | A/mallard/Interior Alaska/10BM01929/2010 (H10) |
| H7 | A/chicken/BC/CN-6/04 (H7) |
| H15 | A/shearwater/West Australia/2576/1979 (H15) |
| B/Wisc10 (Y) | B/Wisconsin/1/2010 (B-HA) |
| B/Bris08 HA (V) | B/Brisbane/60/2008 (B-HA) |
| B/Mal04 (V) | B/Malaysia/2506/2004 (B-HA) |
| B/Phu13 (Y) | B/Phuket/3073/2013 (B-HA) |
| B/Col17 (V) | B/Colorado/06/2017 (B-HA) |
| B/Lee HA | B/Lee/1940 (B-HA) |

### REFERENCES

1. Worldometer. (2020), vol. 2020.
2. N. Chen et al., Epidemiological and clinical characteristics of 99 cases of 2019 novel coronavirus pneumonia in Wuhan, China: a descriptive study. Lancet 395, 507-513 (2020).
3. S. Tabata et al., Clinical characteristics of COVID-19 in 104 people with SARS-CoV-2 infection on the Diamond Princess cruise ship: a retrospective analysis. The Lancet Infectious Diseases, (2020).
4. N. Zhu et al., A Novel Coronavirus from Patients with Pneumonia in China, 2019. N Engl J Med 382, 727-733 (2020).
5. K. J. Meyers et al., A cross-sectional community-based observational study of asymptomatic SARS-CoV-2 prevalence in the greater Indianapolis area. Journal of Medical Virology, (2020).
6. D. Korean Society of Infectious et al., Report on the Epidemiological Features of Coronavirus Disease 2019 (COVID-19) Outbreak in the Republic of Korea from January 19 to Mar. 2, 2020. J Korean Med Sci 35, e112 (2020).
7. T. M. McMichael et al., Epidemiology of Covid-19 in a Long-Term Care Facility in King County, Wash. N Engl J Med, (2020).
8. J. T. Wu et al., Estimating clinical severity of COVID-19 from the transmission dynamics in Wuhan, China. Nat Med, (2020).
9. J. Ioannidis, medRxiv, (2020).
10. G. Onder, G. Rezza, S. Brusaferro, Case-Fatality Rate and Characteristics of Patients Dying in Relation to COVID-19 in Italy. JAMA, (2020).
11. F. Zhou et al., Clinical course and risk factors for mortality of adult inpatients with COVID-19 in Wuhan, China: a retrospective cohort study. Lancet, (2020).
12. J. L. Casanova, L. Abel, Lethal Infectious Diseases as Inborn Errors of Immunity: Toward a Synthesis of the Germ and Genetic Theories. Annu Rev Pathol, (2020).
13. J. L. Casanova, L. Abel, The human genetic determinism of life-threatening infectious diseases: genetic heterogeneity and physiological homogeneity? Hum Genet 139, 681-694 (2020).
14. J. L. Casanova, H. C. Su, C. H. G. Effort, A Global Effort to Define the Human Genetics of Protective Immunity to SARS-CoV-2 Infection. Cell 181, 1194-1199 (2020).
15. S. Y. Zhang, Q. Zhang, J. L. Casanova, H. C. Su, C. Team, Severe COVID-19 in the young and healthy: monogenic inborn errors of immunity? Nat Rev Immunol, (2020).
16. S. Asgari et al., Severe viral respiratory infections in children with IFIH1 loss-of-function mutations. Proc Natl Acad Sci USA 114, 8342-8347 (2017).
17. I. T. Lamborn et al., Recurrent rhinovirus infections in a child with inherited MDA5 deficiency. J Exp Med 214, 1949-1972 (2017).
18. I. T. Lamborn, H. C. Su, Genetic determinants of host immunity against human rhinovirus infections. Hum Genet 139, 949-959 (2020).
19. H. K. Lim et al., Severe influenza pneumonitis in children with inherited TLR3 deficiency. J Exp Med 216, 2038-2056 (2019).
20. M. J. Ciancanelli et al., Infectious disease. Life-threatening influenza and impaired interferon amplification in human IRF7 deficiency. Science 348, 448-453 (2015).
21. Q. Zhang, Human genetics of life-threatening influenza pneumonitis. Hum Genet, (2020).
22. M. J. Ciancanelli, L. Abel, S. Y. Zhang, J. L. Casanova, Host genetics of severe influenza: from mouse Mx1 to human IRF7. Curr Opin Immunol 38, 109-120(2016).
23. N. Hernandez et al., Life-threatening influenza pneumonitis in a child with inherited IRF9 deficiency. J Exp Med 215, 2567-2585 (2018).
24. V. Sancho-Shimizu et al., Herpes simplex encephalitis in children with autosomal recessive and dominant TRIF deficiency. J Clin Invest 121, 4889-4902 (2011).
25. A. Casrouge et al., Herpes simplex virus encephalitis in human UNC-93B deficiency. Science 314, 308-312 (2006).
26. R. Perez de Diego et al., Human TRAF3 adaptor molecule deficiency leads to impaired Toll-like receptor 3 response and susceptibility to herpes simplex encephalitis. Immunity 33, 400-411 (2010).
27. M. Herman et al., Heterozygous TBK1 mutations impair TLR3 immunity and underlie herpes simplex encephalitis of childhood. J Exp Med 209, 1567-1582 (2012).
28. L. L. Andersen et al., Functional IRF3 deficiency in a patient with herpes simplex encephalitis. J Exp Med 212, 1371-1379 (2015).
29. N. Hernandez et al., Inherited IFNAR1 deficiency in otherwise healthy patients with adverse reaction to measles and yellow fever live vaccines. Journal of Experimental Medicine 216, 2057-2070 (2019).
30. C. J. Duncan et al., Human IFNAR2 deficiency: Lessons for antiviral immunity. Sci Transl Med 7, 307ra154 (2015).
31. S. Dupuis et al., Impairment of mycobacterial but not viral immunity by a germline human STAT1 mutation. Science 293, 300-303 (2001).
32. S. Hambleton et al., STAT2 deficiency and susceptibility to viral illness in humans. Proc Natl Acad Sci USA 110, 3053-3058 (2013).
33. G. Zhang et al., A proline deletion in IFNAR1 impairs IFN-signaling and underlies increased resistance to tuberculosis in humans. Nature communications 9, 85(2018).
34. J. Hadjadj et al., Impaired type I interferon activity and exacerbated inflammatory responses in severe Covid-19 patients. medRxiv, (2020).
35. S. Trouillet-Assant et al., Type I IFN immunoprofiling in COVID-19 patients. J Allergy Clin Immunol, (2020).
36. M. A. DePristo et al., A framework for variation discovery and genotyping using next-generation DNA sequencing data. Nat Genet 43, 491-498 (2011).
37. H. Li, R. Durbin, Fast and accurate short read alignment with Burrows-Wheeler transform. Bioinformatics (Oxford, England) 25, 1754-1760 (2009).
38. T. Gambin et al., Homozygous and hemizygous CNV detection from exome sequencing data in a Mendelian disease cohort. Nucleic Acids Res 45, 1633-1648 (2017).
39. D. Backenroth et al., CANOES: detecting rare copy number variants from whole exome sequencing data. Nucleic Acids Res 42, e97 (2014).
40. G. Kerner et al., Homozygosity for TYK2 P1104A underlies tuberculosis in about 1% of patients in a cohort of European ancestry. Proc Natl Acad Sci USA 116, 10430-10434 (2019).
41. S. Belkaya et al., Autosomal Recessive Cardiomyopathy Presenting as Acute Myocarditis. J Am Coll Cardiol 69, 1653-1665 (2017).
42. P. Meade et al., Influenza Virus Infection Induces a Narrow Antibody Response in Children but a Broad Recall Response in Adults. mBio 11, (2020).

### Example 2

### Auto-Antibodies Against Type I Ifns in Patients with Severe Covid-19

Interindividual clinical variability in the course of SARS-CoV-2 infection is immense, ranging from silent infection to lethal disease. We report that 55 of 603 patients (9.1%) with life-threatening COVID-19 pneumonia had high titers of IgG auto-Abs against 7 of the 17 type I IFNs, including 6 of the 11 widely functional IFNs (5 IFN-α and IFN-ε), at the onset of their critical illness. The auto-Abs neutralize IFN-α2 in vitro, including against anti-SARS-CoV-2, while the 13 IFN-α, of which 7 are widely functional, are undetectable in the serum in vivo. No such auto-Abs are found in 370 individuals with asymptomatic or mild SARS-CoV-2 infection and 400 healthy controls. All but seven of the patients with auto-Abs are male (85%), and older than male patients without auto-Abs. Moreover, one of the female patients has X-linked incontinentia pigmenti (IP) and a third of uninfected women with IP tested also have such auto-Abs. Finally, four patients with other, life-threatening viral diseases display such auto-Abs. At least 9% of patients and 10.5% of men with life-threatening COVID-19 pneumonia have an X-linked, age-dependent auto-immune phenocopy of autosomal inborn errors of type I IFN immunity. Treatment with IFN-β, plasmapheresis, B-cell depletion, or the inhibition of type I IFN-reactive B cells may benefit these patients.

Three life-threatening infectious diseases can be driven by monogenic inborn errors of cytokine immunity or their auto-immune phenocopies (1). Like inborn errors of IFN-γ immunity, auto-Abs against IFN-γ underlie mycobacterial disease (2-4). Both inborn errors of IL-17A/F immunity and auto-Abs against IL-17A or IL-17F underlie chronic mucocutaneous candidiasis (5, 6). Patients with inborn errors of IL-6 immunity or auto-Abs against IL-6 suffer from staphylococcal disease (7). Anti-cytokine auto-Abs can be genetically driven, as illustrated by auto-Abs against IL-17A/F, which are driven by mono- or biallelic mutations in AIRE and autoimmune polyendocrine syndrome type 1 (APS-1) (5, 6, 8, 9), and by auto-Abs against IFN-γ, which are driven with lower penetrance by heterozygosity or homozygosity for HLA-DRB1 1502 or 1602 (10-12). Neutralizing auto-Abs against type I IFNs have long been known to be present in patients treated with IFN-α (13, 14) and in almost all patients with APS-1 (15, 16). They are also seen in women with systemic lupus erythematosus (17, 18, 18, 19) and patients with Down syndrome (unpublished). It is intriguing that these patients do not apparently suffer from unusually severe viral infections, as Ion Gresser described a patient with unexplained auto-Abs against type I IFNs suffering from severe varicella and zoster in 1985 (20). Moreover, auto-Abs against type I IFNs in patients homozygous for hypomorphic RAG1 or RAG2 mutations have been associated with severe viral disease, including varicella, and RSV or adenovirus pneumonia (21). Finally, human inborn errors of type I IFNs underlie severe viral diseases, respiratory and otherwise (22-29). In this context, our attention was recently drawn to three APS-1 patients with life-threatening COVID-19 pneumonia (30) (unpublished data). Moreover, patients with Down syndrome (DS), who often carry auto-Abs against type I IFNs (31), were also recently reported to suffer from severe COVID-19 pneumonia (32). While searching for inborn errors of type I IFNs in patients with severe COVID-19 pneumonia (33) (accompanying report), we hypothesized that neutralizing auto-Abs against type I IFNs might also underlie severe COVID-19 pneumonia.

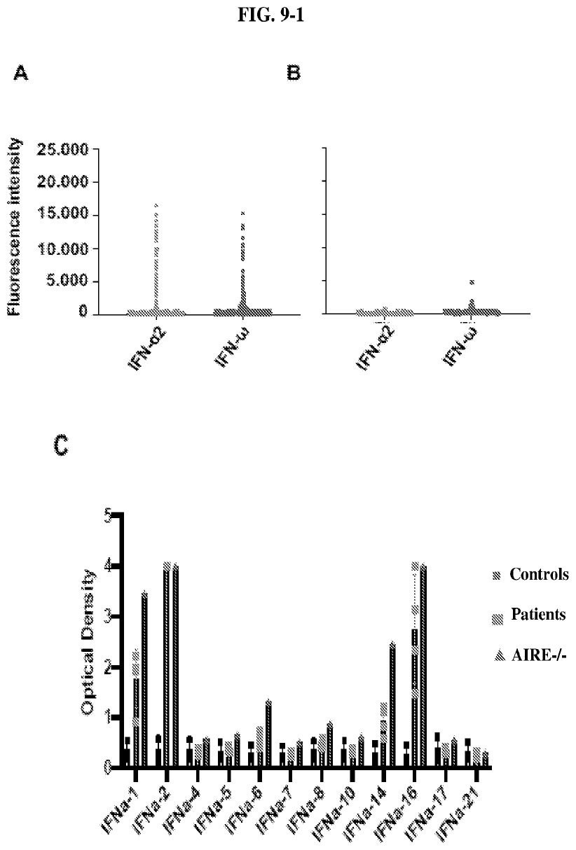

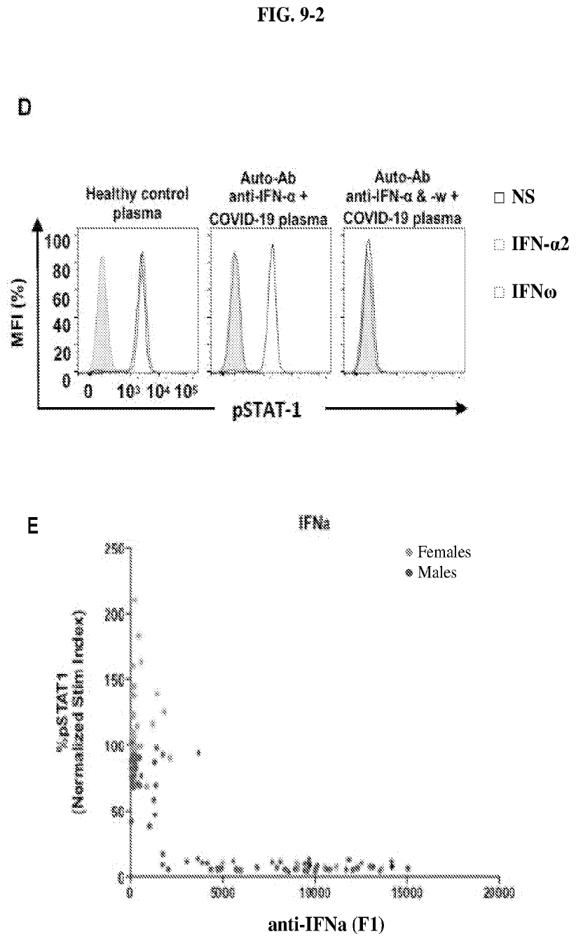

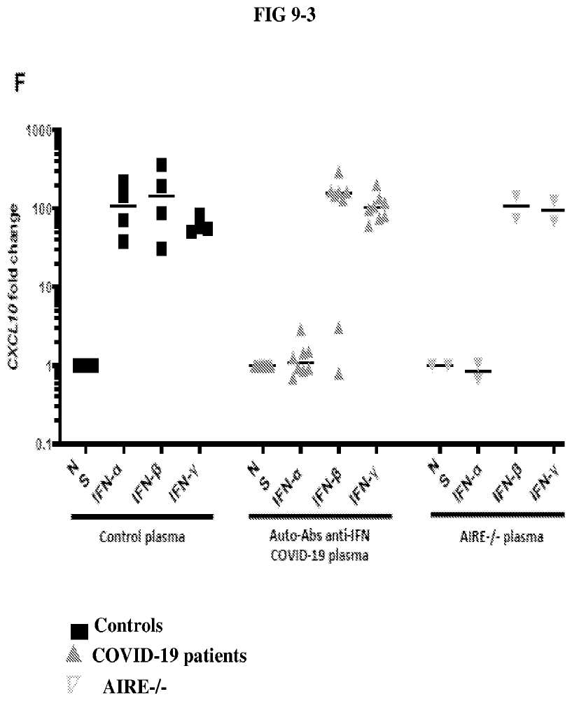

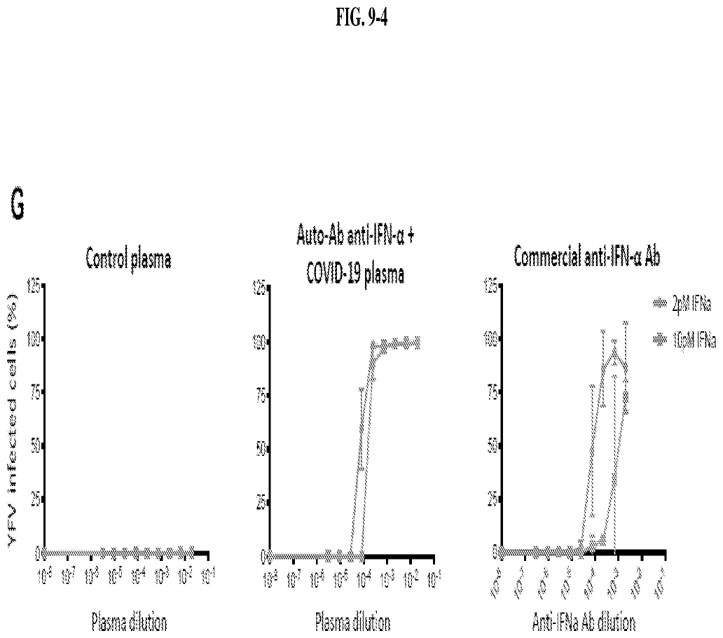

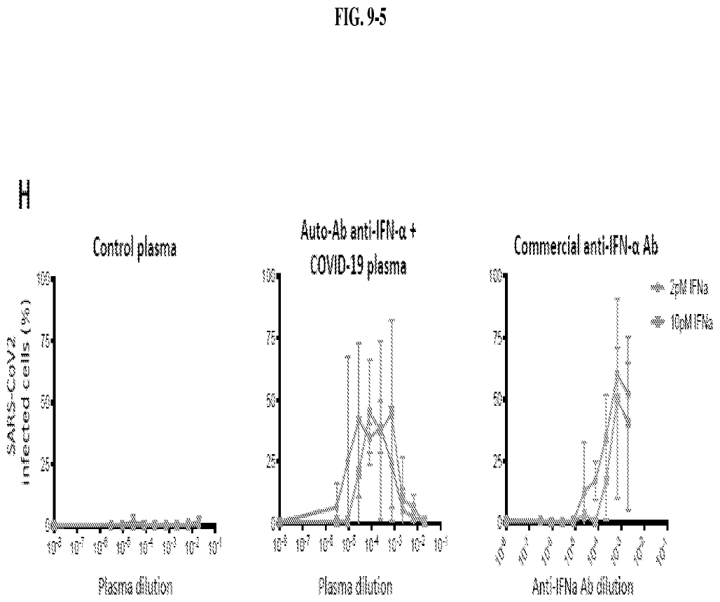

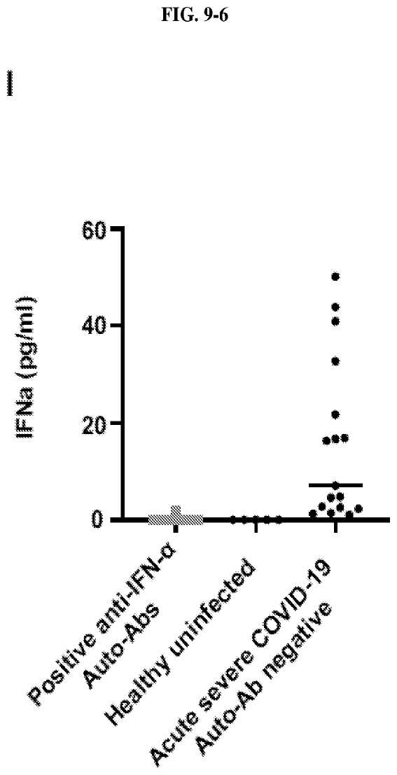

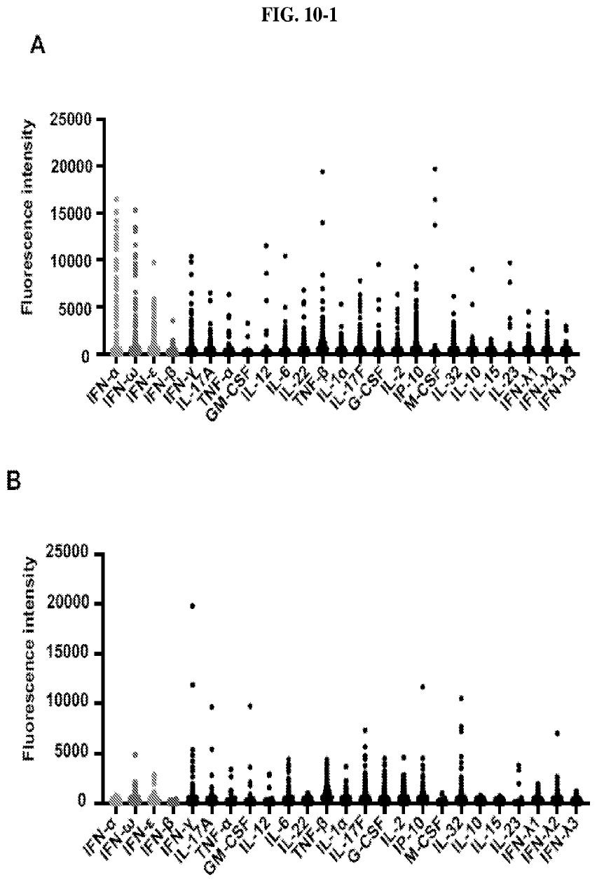

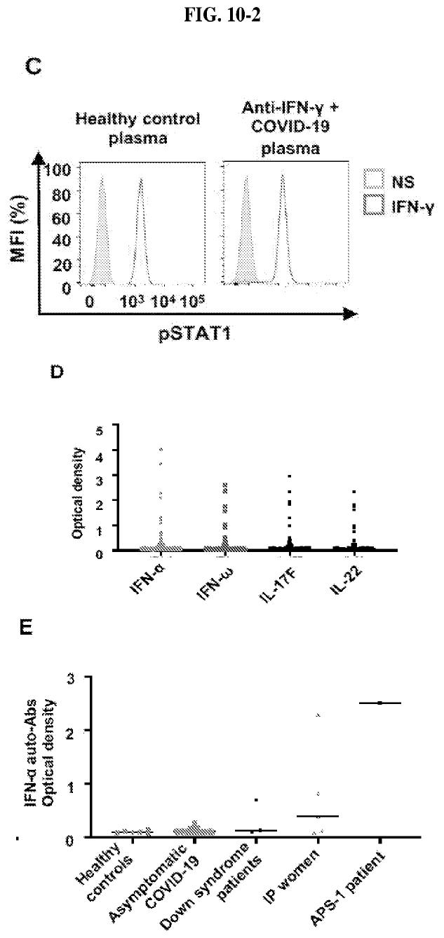

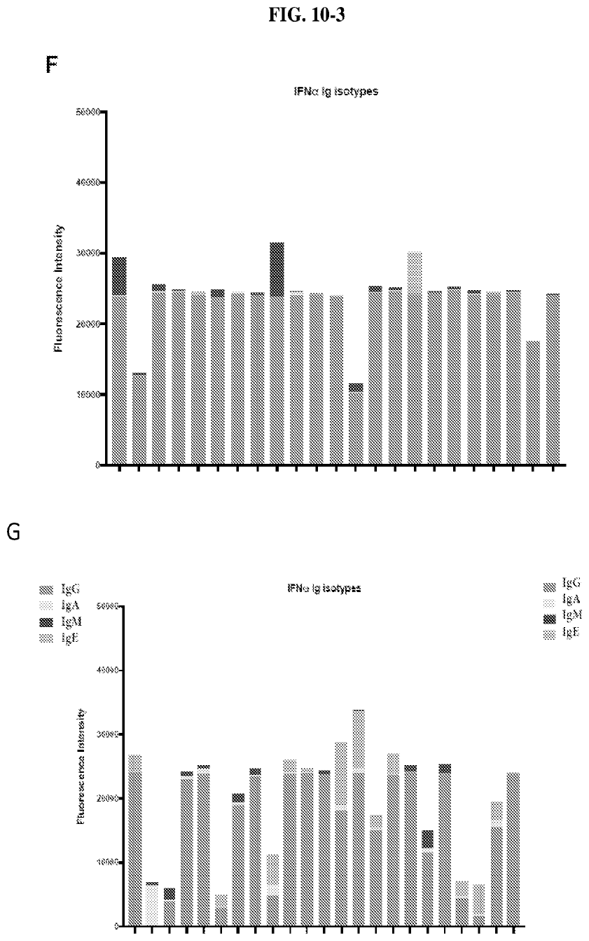

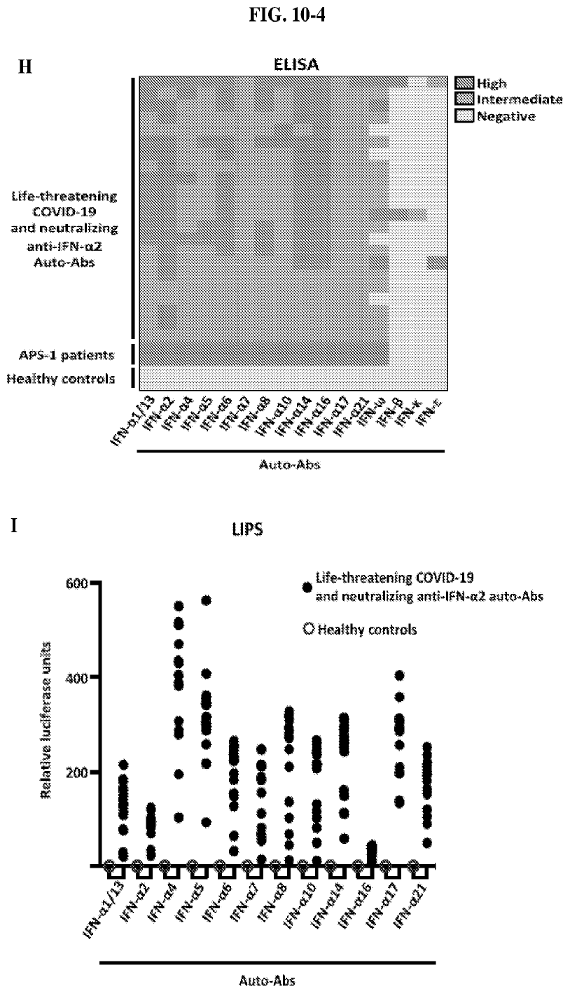

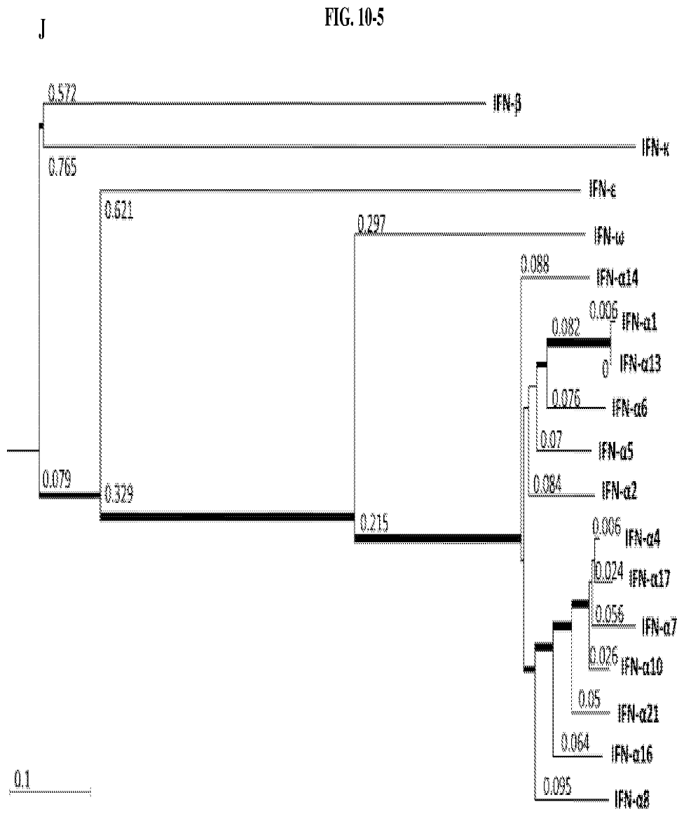

We searched for auto-Abs against type I IFNs in 603 hospitalized patients with life-threatening COVID-19 pneumonia (as defined in M&M), 400 healthy controls not infected with SARS-CoV-2, 370 individuals infected with SARS-CoV-2 and asymptomatic or with mild disease, and 120 patients hospitalized for other severe viral infections (TABLE 4). Plasma or serum samples were collected from patients with severe COVID-19 on the first days of hospitalization and during the acute phase of the disease. Multiplex particle-based flow cytometry revealed a high fluorescence intensity (FI; >4,000, for IFN-α2 and >1,000 for IFN-ω, definitions based on correlation with neutralizing effect from previous experiments) of IgG auto-Abs against IFN-α2 and/or IFN-ω in 62 patients (10.2%) with severe forms of COVID-19 (FIG. 9, A). In the same assay, some patients also had auto-Abs against other cytokines (IFN-γ, IL-6, IL-10, IL-22, IL-17F and/or G-CSF), but none of these Abs were neutralizing (FIGS. 10A, B and C). Of the 62 patients with high titers of anti-IFN-α2 and/or anti-IFN-ω auto-Abs, 30 were positive for antibodies against both IFN-α2 and IFN-ω, ten were positive for antibodies against IFN-α2 only and 22 were positive for antibodies against IFN-ω only. Six women (excluding a woman with IP) tested positive for auto-Abs: 4 had both anti-IFN-α2 and anti-IFN-ω antibodies, whereas two had only anti-IFN-ω auto-Abs. None of the 770 individuals from any of the two control groups (asymptomatic/mild infected or healthy) were positive for either type of auto-Ab, except for one woman who tested positive for auto-Abs against IFN-ω (FIG. 9B).

TABLE 4
Age and Gender of the patients and controls cohorts

| | | Male/Female | Mean |
|---|---|---|---|
| | Number | ratio | Age (SD) |
| Severe COVID-19 patients | 55 | 5.6 | 61.4 (14.3) |
| with Auto-Abs | | | |
| Severe COVID-19 patients | 541 | 3.3 | 58.2 (15.7) |
| Without Auto-Abs | | | |
| Asymptomatic or pauci- | 370 | 0.3 | 41.8 (14.6) |
| symptomatic infected with | | | |
| SARS-Cov2 | | | |
| Other severe viral disease | 120 | 1.0 | 18.2 (15.8) |

For IFN-α2 and IFN-ω, we also ran classical enzyme-linked immunosorbent assays (ELISA), with plasma samples with a dilution factor twice that used in the multiplex assay. Of the 350 patients tested, 10% were positive for IFN-α2 and/or IFN-ω, yielding results matching those of the other assay, and the discrepancy rate was 5% (FIG. 10D, E). We also found that most patients tested (N=22) had low titers of IgA auto-Abs against IFN-α2 and/or IFN-ω, while some had IgM auto-Abs (FIG. 10F, G). Finally, we investigated whether the patients with auto-Abs against IFN-α2 and/or IFN-ω also had auto-Abs against some of the other 15 type I IFNs. Interestingly all the patients tested (N=5) with auto-Abs against IFN-α2 also had auto-Abs against 6 other IFN-α subtypes (IFN-α1, -α2, -α6, -α13, -α14, -α16) (FIG. 9C). Of note, 4 of these are among the 7/11 type I IFNs that are widely functional in the general population, having evolved under strong purifying selection (34). They also cluster in two main phylogenetic branches (FIG. 10H). Only 1/62 patient also had auto-Abs against IFN-β while 7/62 (11%) had auto-Abs against IFN-ε (FIG. 10A). We did not detect auto-Abs against IFN-α4, IFN-α5, IFN-α7, IFN-α8, IFN-α10, IFN-α17 and IFN-α21 in the patients tested (FIG. 9C). Overall, we found that at least 10.2% of patients with severe COVID-19 pneumonia had high levels of IgG auto-Abs against at least one type I IFN, while all tested had auto-Abs against several of the 17 type I IFNs. In two unrelated patients for whom we had plasma drawn prior infection with SARS-CoV-2, we detected the auto-Abs against type I IFNs, indicating that they were not triggered by, but pre-existed infection with SARS-CoV-2. We further tested this possibility, by screening for auto-Abs against type I IFNs in 120 patients with other severe viral infections, including severe viral respiratory infections. Interestingly, we found that four (2 men, 2 women) displayed auto-Abs against IFN-α2 and IFN-ω (TABLE 5). Two children had suffered from severe viral pneumonia, including acute respiratory distress syndrome caused by influenza virus in one and recurrent severe pneumonia caused by various viruses, including common coronavirus, respiratory syncytial virus (RSV), and rhinovirus in another.

Two unrelated adults had suffered from severe clinical disease following inoculation with live attenuated yellow fever virus vaccine (YFV). One of these patients reacting to YFV is a woman who was later diagnosed with systemic lupus erythematosus (SLE). SLE patients are known to have high levels of IFN-α (35), and auto-Abs against type I IFN have been described in SLE (17-19, 36). The observation of these 4 patients is important for several reasons. First, it suggests that various severe viral diseases, other than severe COVID-19 pneumonia, can be favored by auto-Abs against type I IFNs. Second, it suggests that the auto-Abs seen in COVID-19 patients were probably present before SARS-CoV-2 or any other viral infection, and not triggered by these viruses, as neatly illustrated by the adverse reaction to YFV, which occurred in the days following vaccination. Third, it validates the notion that auto-Abs against type I IFN can neatly phenocopy disorders of the three IFN-related pathways selected for the search for autosomal inborn errors of IFN immunity in the accompanying report: the TLR3 pathway for severe influenza pneumonia (28), the MDA5 pathway for RSV and related viruses, including common coronaviruses (37, 38), and the IFNAR1/IFNAR2 pathway for YFV-associated disease (22, 23).

TABLE 5
Clinical Information of the 4 Patients with
other Viral Diseases and Positive Auto-Abs

| Age | Gender | Virus/Disease |
|---|---|---|
| 62 | M | YEL-AVD |
| 50 | F | YEL-AVD + SLE |
| 6 | F | Flu/ARDS |
| 10 | F | Multiple infections |
| | | to respiratory viruses |
| | | (RSV, Flu) |

YEL-AVD: Yellow-fever vaccine-associated viscerotropic disease;
SLE: Systemic lupus erythematosus;
ARDS: Acute respiratory distress syndrome;
Flu: influenza;
RSV: respiratory syncytial virus.

We then tested whether the auto-Abs against IFN-α2 and IFN-ω were neutralizing. We incubated PBMCs from healthy controls with 10 ng/mL IFN-α2 or IFN-w in the presence of plasma from healthy individuals or from patients with auto-Abs. Complete abolition of pSTAT1 induction was observed in all patients with high titers of auto-Abs against IFN-α2 tested (N=31) confirming the relevance of the threshold that was used (FIG. 9D, E) and that the 9 remaining patients to be tested should be neutralizing and were thus considered as such. We also screened 58 patients with intermediate titers of auto-Abs against IFN-α2 and identified an additional 5 patients with neutralizing auto-Abs against IFN-α2. For IFN-ω, 32 out of 61(52%) patients that we tested had neutralizing auto-Abs. Overall, we identified 55 patients with neutralizing auto-Abs (9.2% of severe COVID patients) against IFN-α2 and IFN-ω (N=42), IFN-α2 only (N=11) or IFN-ω only (N=2) (TABLE 6). We also analyzed interferon-stimulated gene (ISG) induction after 2 h of stimulation with IFN-α2 or IFN-γ. With plasma of twelve patients with auto-Abs against IFN-α2, the induction of CXCL10 was abolished after IFN-α2 stimulation but maintained after stimulation with IFN-γ(FIG. 9F). Interestingly, serum from the patient with high titers auto-Abs against IFN-β showed neutralizing activity against IFN-β. Finally, we tested if the plasmas from patients with auto-Abs against type I IFN were neutralizing for their anti-viral activity. We found that all 5 tested plasmas of patients with neutralizing auto-Abs neutralized the protective activity of IFN-α2 in vero cells infected with vesicular stomatitis virus. Moreover, we found that the two plasma of patients with neutralizing auto-Abs tested did neutralize the activity of IFN-α2 in ACE2-expressing human fibroblasts infected with YFV-17D or SARS-CoV-2 (FIG. 9G, H). Conversely, the plasmas of healthy controls or SARS-CoV-2 infected patients without auto-Abs did not block the protective effect of IFN-α2. These data provided compelling evidence that the patients' blood carried titers of auto-Abs that were neutralizing of the corresponding type I IFNs for their activity against SARS-CoV-2.

Neutralizing antibodies against both IFN-α2 and IFN-ω were detected in 52 of 101 patients (51%), against only IFNα2 in 36 patients (36%), and against only IFNω in 13 patients (13%) at the IFN-α2 and IFN-w concentrations tested. IgG depletion from patients with auto-Abs restored normal pSTAT1 induction after IFN-α2 and IFN-co stimulation, whereas the purified IgG fully neutralized this induction (FIG. 9D and data not shown). Furthermore, these auto-Abs neutralized high amounts of IFN-α2 and were neutralizing at high dilutions. Notably, 15 patients with life threatening COVID-19 and auto-Abs against IFN-α2 and/or IFN-w also had auto-Abs against other cytokines [IFN-γ, granulocyte-macrophage colony-stimulating factor (GM-CSF), IL-6, IL-10, IL-12p70, IL-22, IL-17A, IL-17F, and/or tumor necrosis factor-3 (TNF[3)], only three of which (IL-12p70, IL-22, and IL-6) were neutralizing (in four patients). Similar proportions were observed in the other cohorts (data not shown).

We also analyzed ISG induction after 2 hours of stimulation with IFN-α2, IFN-β1, or IFN-γ in the presence of plasma from healthy individuals or from patients with auto-Abs. With plasma from eight patients with auto-Abs against IFN-α2, the induction of ISG CXCL10 was abolished after IFN-α2 stimulation but maintained after stimulation with IFN-γ. We then found that plasma from the five patients with neutralizing auto-Abs neutralized the protective activity of TFN-α2 in Madin-Darby bovine kidney (MDBK) cells infected with vesicular stomatitis virus (VSV). Overall, we found that 101 of 987 patients (10.2%)—including 95 men (94%)—with life-threatening COVID-19 pneumonia had neutralizing IgG auto-Abs against at least one type I IFN. By contrast, auto-Abs were detected in only 4 of 1227 healthy controls (0.33%) (Fisher exact test, P<10⁻¹⁶) and in none of the 663 patients with asymptomatic or mild SARS-CoV-2 infection tested (Fisher exact test, P<10⁻¹⁶),

TABLE 6
Clinical and Epidemiological Characteristics of the 46 Patients
with COVID-19 Infection and Auto-Abs Against type I IFNs

| Indi- | | | | Co- | |
|---|---|---|---|---|---|
| vid- | | | COVID | morbid- | |
| uals | Age | Sex | Severity | ities | Details |
| 1 | 60 | M | Severe | Y | CVID |
| 2 | 58 | M | Severe | Y | HTA, diabetes, cardiovascular, |
| | | | | | respiratory, obesity |
| 1 | 69 | M | Severe | Y | HTA |
| 4 | 64 | M | Severe | Y | HTA, cardiovascular |
| 5 | 61 | M | Severe | Y | HTA |
| 6 | 41 | M | Severe | Y | Psoriatic arthritis |
| 7 | 68 | M | Severe | Y | HTA, diabetes, cardiovascular |
| 8 | 74 | M | Severe | Y | Respiratory |
| 9 | 73 | M | Severe | Y | Diabetes, obesity |
| 10 | 69 | M | Severe | Y | HIV |
| 11 | 67 | M | Severe | Y | HTA, cancer |
| 12 | 48 | M | Severe | NA | NA |
| 13 | 70.4 | M | Severe | Y | HTA, diabetes, obesity |
| 14 | 52 | M | Severe | Y | Uveitis, psoriasis |
| 15 | 56 | M | Severe | Y | Autoimmunity |
| 16 | 88 | F | Severe | Y | HTA, cardiovascular |
| 17 | 74 | M | Severe | N | |
| 18 | 64 | M | Severe | Y | HTA, cardiovascular, cancer |
| 19 | 67 | F | Severe | Y | HTA, diabetes, cardiovascular, |
| | | | | | respiratory |
| 20 | 71.6 | F | Severe | Y | HTA, cardiovascular |
| 21 | 53 | M | Severe | Y | Cancer, neurology |
| 22 | 69 | M | Severe | Y | HTA |
| 23 | 74 | M | Severe | Y | HTA, cardiovascular |
| 24 | 32 | F | Severe | Y | Incontinentia Pigmenti |
| 25 | 25 | M | Severe | N | |
| 26 | 45 | M | Severe | N | |
| 27 | 73.9 | M | Severe | | HTA, diabetes |
| 28 | 66.1 | M | Severe | N | |
| 29 | 71.2 | F | Severe | Y | Cardiovascular, cancer |
| 30 | 44 | M | Severe | N | |
| 31 | 25 | M | Severe | N | |
| 32 | 68.4 | M | Severe | N | |
| 33 | 79.9 | M | Severe | Y | HTA, diabetes, cardiovascular |
| 34 | 25 | M | Severe | Y | Obesity |
| 35 | 75 | M | Severe | Y | HTA, cancer |
| 36 | 62 | M | Severe | Y | HTA, obesity |
| 37 | 54.4 | M | Severe | N | |
| 38 | 67.3 | M | Severe | Y | HTA, diabetes |
| 39 | 70 | M | Severe | Y | Diabetes, respiratory |
| 40 | 70 | M | Severe | Y | Cardiovascular |
| 41 | 70 | F | Severe | Y | HTA, obesity |
| 42 | 58 | M | Severe | Y | HTA |
| 43 | 63 | M | Severe | Y | HTA |
| 44 | 63 | M | Severe | Y | HTA, diabetes, cardiovascular, |
| | | | | | respiratory |
| 45 | 65 | M | Severe | Y | HTA, diabetes, cardiovascular, |
| | | | | | kidney transplant |
| 46 | 61 | F | Severe | Y | Respiratory |

CVID: Common variable immunodeficiency;
HTA: Hypertension;
Cardiovascular: cardiovascular disease;
Respiratory: respiratory disease;
Obese: Body-mass index >30;
NA: Non-applicable.

Recent studies have reported that some patients with COVID-19, including patients with severe disease, have low or undetectable IFN-α levels during SARS-CoV-2 infection, as shown by Simoa digital ELISA, which measures the levels of the 13 IFN-αtypes (39, 40) (unpublished). Low levels of these IFN may be a cause or consequence of disease or severe disease. We thus assessed 12 patients who were studied here and previously found to have low or undetectable levels of serum IFN-α, including some of the patients reported in these papers (39,41); we found that 42% (N=5) had auto-Abs against type I IFNs. We are investigating whether and how many of the remaining 7 have inborn errors of type I IFNs (as described in Example 1). Moreover, we found that all patients with neutralizing auto-Abs against type I IFNs tested (N=25) also had undetectable levels of IFN-α in their plasma (FIG. 9I). Another 5 patients, with positive auto-Abs against IFN-α2, not yet tested for their neutralization capacity, had undetectable serum IFN-α levels, while one was low. The presence of circulating neutralizing auto-Abs against type I IFNs is therefore strongly associated with low serum IFN-αlevels (p<10⁻⁶). Other patients with low or undetectable levels of serum IFN-α may still have auto-Ab levels but below the limit of detection in this study, or undetectable because bound to IFN-α, or have genetic defects affecting IFN production or amplification other than those reported in the accompanying paper. Our findings indicate that the auto-Abs against type I IFNs present in these patients were also neutralizing in vivo.

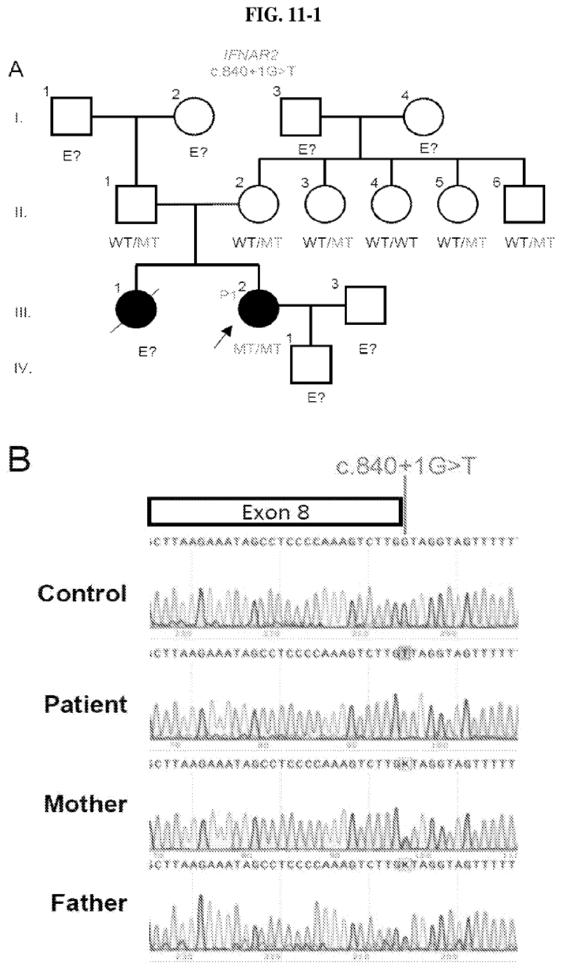

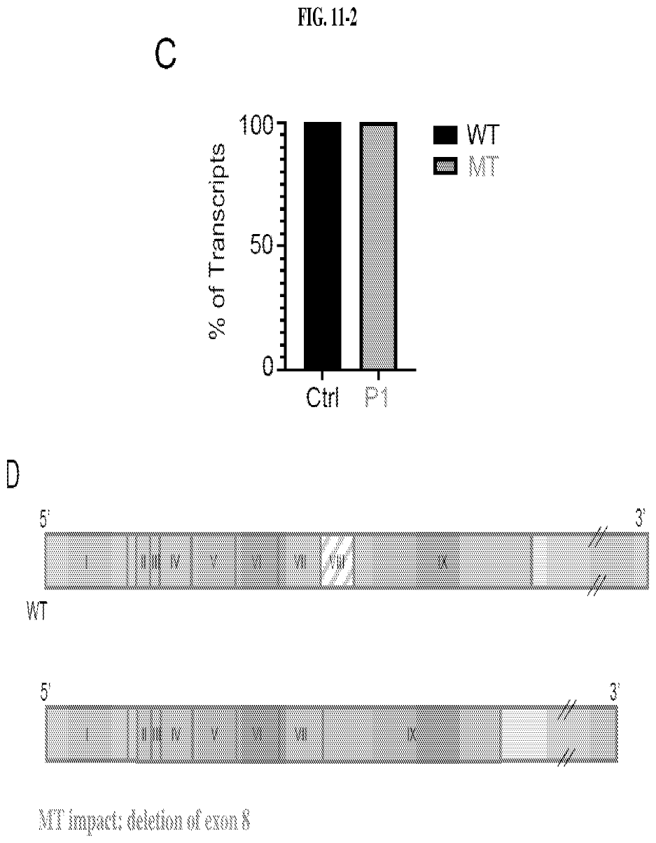

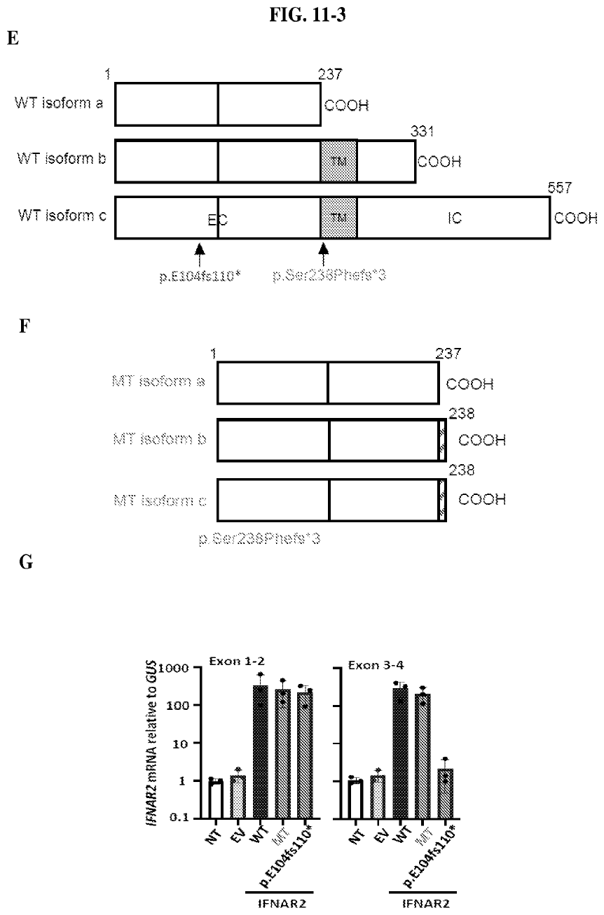

We performed whole-exome sequencing (WES) or whole-genome sequencing (WGS) on 35 of the 55 patients with high levels of neutralizing auto-Abs against type I IFNs. Various HLA alleles have been associated with autoimmune diseases, including the production of anti-IFN-g auto-Abs (10-12). We therefore inferred HLA data from WES or WGS data (42). We were unable to identify a specific haplotype either common to all patients with auto-Abs against type I IFNs or even enriched in these individuals. Nevertheless, there was a strong excess of male patients (49 of 55, 89%) with severe COVID-19 pneumonia and auto-Abs against type I IFNs. This proportion of males was slightly higher than that observed in severe patients without auto-Abs (76%; p=0.028), yet, importantly, much greater than that of male patients within the asymptomatic or pauci-symptomatic cohort (28%, p<10⁻⁶). Moreover, one of the female patients had X-linked incontinentia pigmenti (IP) due to a large heterozygous deletion in NEMO. All cells in women with IP, whose prevalence is about 1/1,000,000, express the same single X chromosome (43-45). We tested 13 other women with IP who had not been infected with SARS-CoV-2. Strikingly, we found high titers of auto-Abs against IFN-α2 in 30% of these women (FIG. 11E). This suggested that the auto-Abs against type I IFNs in the woman with IP and severe COVID-19 were present prior to infection with SARS-CoV-2, and that women with IP, like women with SLE or patients with DS, are at much higher risk of displaying such auto-Abs than the general population. The high male preponderance in auto-Ab production and their frequent occurrence in women with IP (46, 47) suggest that the production of auto-Abs against type I IFNs can be X-linked recessive, at least in a subset of patients.

Using the WGS data of male patients with (N=29) and without (N=242) auto-Abs, we found no single-nucleotide variants (SNV) or copy-number variants (CNV) located on the X significantly associated, at the chromosome-wide level, with the presence of auto-Abs. There was no variant significantly associated on the Y chromosome either. This result suggests that the pathogenesis of auto-Ab production if X-linked, is not monogenic with homogeneity. Alternative plausible genetic models of X-linked inheritance include (i) monogenic inheritance with heterogeneity, and (ii) digenic inheritance with heterogeneity. A gene dosage mechanism involving a large number of loci on the X chromosome may also be involved, as suggested by the presence of auto-Abs in four of the 13 women with IP tested. Women (XX) appear to be protected, with the exception of women with IP, SLE, of DS, and perhaps those with early skewed X inactivation in the course of development, whereas a larger fraction of men (XY) develop auto-Abs against type I IFNs. These findings contrast with the usual epidemiological distribution of autoimmunity. Based on the absence of auto-Abs in 200 healthy males, the prevalence of auto-Abs against type I IFNs in the general male population is probably <1%, lower than that in this population of patients with severe COVID-19 pneumonia by a factor of at least 10. The patients tested positive for auto-Abs were slightly older than the rest of our cohort (64% of patients' positives for auto-Abs were over 60 years old versus 42% in the rest of the cohort, p=0.0064), suggesting that the frequency of auto-Ab production increases with age. Auto-Abs were however found from age 25 to 88 years old. Of the 36 patients for whom we performed PCA analysis, 32 were European, 1 was North-African, 1 South-Asian and 1 Latino. Large-scale studies will be required to determine the frequency of such auto-Abs in humans of different ages and ancestries.

We provide evidence that the auto-Abs against type I IFNs observed in these 55 patients were not triggered by SARS-CoV-2, but preceded the infection with this virus, and made a significant contribution to the severity of the disease. First, we obtained a plasma sample taken before COVID-19 infection for two patients, which showed that the auto-Abs were already present before infection. Second, 30% of uninfected women with IP had auto-Abs against type I IFNs, including one who developed life-threatening COVID-19 upon infection. Third, three patients with AR APS-1 and high titers of neutralizing auto-Abs had severe COVID-19. Fourth, patients with DS more frequently suffer from severe COVID-19 than the general population (32) (unpublished). Fifth, many of our patients also had auto-Abs against other cytokines, suggestive of an underlying auto-immune process, rather than a response to the virus, although past and present viral infections may have increased anti-IFN auto-Ab production. Sixth, IFN-α were undetectable in the serum of the patients with these auto-Abs on the first days of hospitalization, suggesting a pre-existing biological impact in vivo. Seventh, it is difficult to conceive that patients could both break self-tolerance and mount high titers of neutralizing IgG and IgA auto-Abs against type I IFN within only one week of infection with the virus, or even two weeks. Eighth, inborn errors of type I IFNs underlying severe COVID-19 in other previously healthy adult patients are reported in an accompanying paper. Ninth, four patients with other severe viral infections related to type I IFN immunity also have auto-Abs against type I IFNs, including a woman with SLE. Tenth, there is a strong bias in favor of men (p<10⁻⁶), suggesting that the production of auto-Abs to type I IFNs is genetically driven and X-linked recessive, at least in some patients. Collectively, these findings provide compelling evidence that auto-Abs to type I IFNs are a cause, and not a consequence of severe SARS-Cov-2 infection.

We thus report that at least 10% of patients with life-threatening COVID-19 pneumonia have X-linked neutralizing auto-Abs against type I IFNs. The prior Example 1 shows that another 10% of patients carry known or novel forms of autosomal inborn errors of 13 type I IFN-related genes. These two studies highlight the crucial role of type I IFNs in protective immunity against SARS-CoV-2, and other viruses, including respiratory viruses in particular. Our study also reveals a fourth example of auto-Abs against cytokines mimicking inborn errors of the corresponding cytokine, after IFN-γ IL-6, and IL-17A/F (1). The auto-Abs against type I IFNs were probably clinically silent until the patients were infected with SARS-CoV-2, which has been shown to be a poor inducer of type I IFN (48, 49), making the small amounts of IFN produced even more important for protective immunity. The neutralizing auto-Abs against type I IFNs, like autosomal inborn errors of type I IFN production, tip the balance in favor of the virus, resulting in a devastating pneumonia. This report also provides a first compelling explanation for the major sex bias observed in patients with severe COVID-19, and perhaps also the increased risk with age, while offering a possible explanation for geographic disparities in the severity of COVID-19. The penetrance of neutralizing auto-Abs against type I IFNs for severe COVID-19 is probably high upon infection with SARS-CoV-2 and inoculation with the YFV vaccine, while it is much lower upon infection with other, less virulent respiratory viruses. Our findings thus already have clinical implications. First, it is possible to screen SARS-CoV-2-infected patients to identify individuals at risk. Second, this observation paves the way for therapeutic intervention. Although high-dose intravenous IgG apparently helped some patients with severe COVID-19 pneumonia (50), this immunosuppressive approach might be irrelevant to the pathogenic auto-Abs against type I IFNs (51), and pharmaceutical immunoglobulins could also contain certain amounts of anti-cytokine neutralizing antibodies (52). Plasmapheresis or monoclonal Abs depleting plasmablasts (daratumumab) would be better options, if SARS-CoV-2-specific plasma or monoclonal Abs can be used to compensate for the loss of the patient's Abs against the virus (53). Alternatively, one may consider a specific inhibition of type I IFN-reactive B cells by cross-linking their IFN-specific surface Ig with their inhibitory FcRIIb (51). By contrast, early treatment with IFN-α is unlikely to be beneficial, given the high titers of neutralizing auto-Abs, and it might even select B cells producing Auto-Abs with higher affinity to type I IFNs; yet, treatment with IFN-β in patients without auto-Abs against IFN-β could be beneficial. Convalescent plasma therapy (CPT) has also been proposed and administered to a small number of COVID-19 patients (54, 55). We suggest that anti-type I IFN auto-Ab levels should be measured in the convalescent plasma preparations currently being tested in clinical trials, or at least that patients with severe COVID-19 should be excluded. Finally, recombinant IFN-α2, intramuscularly injected or inhaled, may be beneficial in a second step, once the auto-Abs have been depleted by plasmapheresis or the auto-reactive B cells inhibited or depleted.

Materials and Methods

603 patients with proven severe COVID-19 infection, 370 asymptomatic or pauci-symptomatic individuals with proven COVID-19 infection, 350 healthy controls and 120 patients with other severe viral diseases were enrolled in this study, with informed consent and approval obtained from the Necker Hospital and Medical School Institutional Review Board (IRB), the Rockefeller University IRB, the IRB of ASST Ospedale San Gerardo—University of Milano-Bicocca, Monza (Italy) and the IRB of Fondazione IRCCS Policlinico San Matteo, Pavia (Italy). Some patients were enrolled in the French COVID cohort (clinicaltrials.gov NCT04262921). Ethics approval was obtained from the CPP IDF VI (ID RCB: 2020-A00256-33). Some contact subjects were enrolled in the Cov-Contact cohort (clinicaltrials.gov NCT04259892). De-identified samples were studied at the NIAID under non-human subjects research conditions; no additional IRB consent was required at the NIH.

Plasma and serum samples from the patients and controls were frozen at −20° C. immediately after collection. The fluid-phase LIPS assay was used to determine the levels of antibodies against the SARS-CoV-2 nucleoprotein and spike protein, as has been previously described (Burbelo. P D et al (2020) J Infect Dis 222, 206-213).

Serum/plasma samples were screened for autoantibodies against 25 targets in a multiplex particle-based assay, in which magnetic beads with differential fluorescence were covalently coupled to recombinant human protein (2.5 μg/reaction). Beads were combined and incubated with 1:100 diluted serum/plasma samples for 30 minutes. They were then washed and incubated with PE-labeled goat anti-human IgG (1 μg/mL) for an additional 30 minutes. Beads were then washed again and run on a BioPlex X200 instrument in a multiplex assay. Patients with an FI of >1500 for IFN-α2 or IFN-β or >1000 for IFN-ω were tested for blocking activity, as were patients positive for another cytokine.

ELISA was performed as previously described (Puel et al., 2008). In brief, 96-well ELISA plates (MaxiSorp; Thermo Fisher Scientific) were coated by incubation overnight at 4° C. with 2 μg/mL rIL-17F, rIL-22, rhIFN-α, and rhIFN-ω (R&D Systems). Plates were then washed (PBS/Tween 0.005%), blocked by incubation with the same buffer supplemented with 5% nonfat milk powder, washed, and incubated with 1:50 dilutions of plasma samples from the patients or controls for 2 h at room temperature (or with specific mAbs as positive controls). Plates were thoroughly washed. Horseradish peroxidase (HRP)-conjugated Fc-specific IgG fractions from polyclonal goat antiserum against human IgG or IgA (Nordic Immunological Laboratories) were added to a final concentration of 2 μg/mL. Plates were incubated for 1 h at room temperature and washed. Substrate was added and OD was measured. A similar protocol was used when testing for the 21 subtypes of IFN-α, except that the plates were coated using cytokines from PBL assay science (catalog #11002-1).

LIPS—Levels of auto-Abs against IFN-α subtypes were measured with LIPS, as previously described (Meyer S et al (2016) Cell 166:582-595). IFN-a1, IFN-a2, IFN-a4, IFN-a5, IFN-a6, IFN-a7, IFN-a8, IFN-a10, IFN-a14, IFN-a6, IFN-a17, and IFN-a21 sequences were transfected in HEK293 cells, and the IFN-a-luciferase fusion proteins were collected in the tissue culture supernatant. For autoantibody screening, serum samples were incubated with protein G agarose beads, and we then added 2×10⁶ luminescence units (LU) of antigen and incubated. Luminescence intensity was measured. The results are expressed in arbitrary units (AU), as a fold-difference relative to the mean of the negative control samples.

The blocking activity of anti-IFNα and anti-IFNω autoantibodies was determined by assessing STAT1 phosphorylation in healthy control cells following stimulation with the appropriate cytokines in the presence of 10% healthy control or patient serum/plasma. Surface-stained healthy control PBMCs (350,000/reaction) were cultured in serum-free RPMI medium with 10% healthy control or patient serum/plasma and were either left unstimulated or stimulated with IFN aor IFN ((10 ng/mL) for 15 minutes at 37° C. Cells were fixed, permeabilized, and stained for intranuclear phopsho-STAT1 (Y701). Cells were acquired on a BD LSRFortessa cytometer with gating on CD14+ monocytes and analyzed with FlowJo software.

The blocking activity of anti-IFNγ-IL-6, -IL-10, and -G-CSF antibodies was determined in a similar manner, by flow cytometry, with the specifications indicated in TABLE 7 below.

TABLE 7

| Cytokine | Incubation | Signaling | | |
|---|---|---|---|---|
| (concentration) | time | target | Cell type | Method |
| IFNγ | 15 min | pSTAT1 | CD14⁺ | FACS, |
| (400 U/mL) | | (Y701) | monocytes | surface and |
| | | | | IC staining |
| G-CSF | 20 min | pSTAT3 | CD15⁺ | FACS, |
| (10 ng/mL) | | (Y705) | neutrophils | surface and |
| | | | | IC staining |
| IL-6 | 20 min | pSTAT3 | CD14⁺ | FACS, |
| (10 ng/mL) | | (Y705) | monocytes | surface and |
| | | | | IC staining |
| IL-10 | 20 min | pSTAT3 | CD14⁺ | FACS, |
| (10 ng/mL) | | (Y705) | monocytes | surface and |
| | | | | IC staining |
| G-CSF | 20 min | pSTAT3 | CD15⁺ | FACS, |
| (10 ng/mL) | | (Y705) | neutrophils | surface and |
| | | | | IC staining |

We demonstrated that the TFN-α and IFN-ω blocking activity observed was due to auto-Abs and not another plasma factor, by depleting IgG from the plasma with a protein G column. Without eluting the IgG, the flow-through fraction (IgG-depleted) was then collected and compared with total plasma in the phospho-STAT1 assay.

The blocking activity of anti-IFN-γ, -GMCSF, -IFN-λ1, -IFN-λ2, -IFN-13, -IL-6, -IL-10, -IL-12p70, -IL-22, -IL-17A, -IL-17F, -TNFα, and -TNFβ antibodies was assessed with the assays outlined, as previously reported (Walter J E et al (2015) J Clin Invest 125:413504148). For the neutralization of ISG induction, PBMCs were left unstimulated or were stimulated for 2 hours with 10 ng/mL IFN-α or 10 ng/mL IFN-γ in a final volume of 100 mL. Realtime quantitative polymerase chain reaction (RT-qPCR) analysis was performed with Applied Biosysters Tagman assays for CXCL10, and the b-glucuronidase (GUS) housekeeping gene for normalization. Results are expressed according to the ΔΔCt method, as described by the manufacturer's kit.

For the neutralization of ISG induction, peripheral blood mononuclear cells (PBMCs) were thawed and kept overnight in X-VIVO20 (Lonza) at 37° C. under an atmosphere containing 5% C02. We added 0.75×10⁶ PBMCs in 40 μL of X-VIVO20 medium to each well of a 96-well U-bottomed plate with 10 μL of the indicated plasma, and the contents of each well were mixed. PBMCs were then left unstimulated or stimulated for two hours with 10 ng/mL IFNα or 10 ng/mL IFNg in a final volume of 100 μL. We extracted mRNA from the cells with a kit, according to manufacturer's instructions (Zymo Research). We used 250 ng of each mRNA preparation for reverse transcription with oligo-DT primers and Superscript II (Thermo Fisher Scientific). Quantitative real-time PCR (RT-qPCR) was performed with Applied Biosystems Taqman assays for CXCL10 and IFIT1, and the β-glucuronidase (GUS) housekeeping gene for normalization. Results are expressed according to the ΔΔCt method, as described by the kit manufacturer.

Serum-IFNα concentrations were determined with Simoa technology as described in (35, 56) with reagents and procedures obtained from Quanterix Corporation (Quanterix Simoa™ IFNα Reagent Kit, Lexington, Mass., USA), according to the manufacturer's instructions. The dynamic range of the assay was 0 to 54.6 pg/mL with a lower limit of detection of 16 fg/mL and a lower limit of quantification of 64 fg/mL.

The seroneutralization assay was performed as previously described (57). In brief, IFN-μ2 incubated with Madin-Darby bovine kidney protects cultured cells against the cytopathic effect of vesicular stomatitis virus (VSV). Serial two-fold dilutions of patients' serum was added in the incubation medium prior to viral challenge. The titer of anti IFN alpha antibodies was defined as the last dilution causing 50% cell death.

The SARS-CoV-2 infection experiments were performed as follows. Huh7.5 cells were seeded in 96-well plates. 24 h later, recombinant IFN-α2 was incubated together with plasma for 1 h at 37° C., using 2 concentrations of IFN-α2 (2 pM and 10 pM). Starting plasma dilution was 1/50 and 3-fold serial dilutions were done. For the commercial anti-IFN-α2 antibody, dilutions started from 1/500. Then, media from the cells was removed and cells were incubated overnight with the plasma+IFN-α2 mixture. The next day, cells were washed to remove anti-SARS-CoV-2 neutralizing antibodies and fresh media was added. They were then infected with SARS-CoV-2. 48 h later, the cells were fixed and stained for SARS-CoV-2 N protein as described elsewhere (53). A similar method was used for YFV-17D, except that cells were fixed at 3 days post-infection.

SARS-CoV-2 strain USA-WA1/2020 was obtained from BET Resources and amplified in Huh7.5 hepatoma cells at 33° C. Viral titers were measured on Huh7.5 cells in a standard plaque assay. Plasma samples or a commercial anti-IFN-2 antibody were serially diluted and incubated with 20 pM recombinant IFN-α2 for 1 hour at 37° C. (starting concentrations: plasma samples=1/100 and anti-IFN-α2 antibody=1/1000). The cell culture medium was then removed and replaced with the plasma- or antibody-IFN-α2 mixture. The plates were incubated overnight, and the plasma- or anibody-IFN-α2 mixture was removed by aspiration. The cells were washed once with phosphate-buffered saline (PBS) to remove potential anti-SARS-CoV-2 neutralizing antibodies, and fresh medium was then added. Cells were then infected with SARSCoV-2 by directly adding the virus to the wells. Cells infected at a high multiplicity of infection (MOI) were incubated at 37° C. for 24 hours, whereas cells infected at a low MOI were incubated at 33° C. for 48 hours. The cells were fixed with 7% formaldehyde, stained for SARS-CoV-2 with an anti-N antibody, imaged, and analyzed as previously described (Robbiani D F et al (2020) Nature 584:437-442).

### REFERENCES

1. C.-L. Ku, C.-Y. Chi, H. von Bernuth, R. Doffinger, Autoantibodies against cytokines: phenocopies of primary immunodeficiencies? Hum. Genet. 139, 783-794 (2020).
2. R. Döffinger, M. R. Helbert, G. Barcenas-Morales, K. Yang, S. Dupuis, L. Ceron-Gutierrez, C. Espitia-Pinzon, N. Barnes, G. Bothamley, J.-L. Casanova, H. J. Longhurst, D. S. Kumararatne, Autoantibodies to interferon-gamma in a patient with selective susceptibility to mycobacterial infection and organ-specific autoimmunity. Clin. Infect. Dis. 38, e10-14 (2004).
3. C. Höflich, R. Sabat, S. Rosseau, B. Temmesfeld, H. Slevogt, W.-D. Docke, G. Grutz, C. Meisel, E. Halle, U. B. Göbel, H.-D. Volk, N. Suttorp, Naturally occurring anti-IFN-gamma autoantibody and severe infections with Mycobacterium cheloneae and Burkholderia cocovenenans. Blood. 103, 673-675 (2004).
4. B. Kampmann, C. Hemingway, A. Stephens, R. Davidson, A. Goodsall, S. Anderson, M. Nicol, E. Schölvinck, D. Relman, S. Waddell, P. Langford, B. Sheehan, L. Semple, K. A. Wilkinson, R. J. Wilkinson, S. Ress, M. Hibberd, M. Levin, Acquired predisposition to mycobacterial disease due to autoantibodies to IFN-gamma. J. Clin. Invest. 115, 2480-2488 (2005).
5. K. Kisand, A. S. Bse Wolff, K. T. Podkrajsek, L. Tserel, M. Link, K. V. Kisand, E. Ersvaer, J. Perheentupa, M. M. Erichsen, N. Bratanic, A. Meloni, F. Cetani, R. Perniola, B. Ergun-Longmire, N. Maclaren, K. J. E. Krohn, M. Pura, B. Schalke, P. Ströbel, M. I. Leite, T. Battelino, E. S. Husebye, P. Peterson, N. Willcox, A. Meager, Chronic mucocutaneous candidiasis in APECED or thymoma patients correlates with autoimmunity to Th17-associated cytokines. J. Exp. Med. 207, 299-308 (2010).
6. A. Puel, R. Döffinger, A. Natividad, M. Chrabieh, G. Barcenas-Morales, C. Picard, A. Cobat, M. Ouachde-Chardin, A. Toulon, J. Bustamante, S. Al-Muhsen, M. Al-Owain, P. D. Arkwright, C. Costigan, V. McConnell, A. J. Cant, M. Abinun, M. Polak, P.-F. Bougneres, D. Kumararatne, L. Marodi, A. Nahum, C. Roifman, S. Blanche, A. Fischer, C. Bodemer, L. Abel, D. Lilic, J.-L. Casanova, Autoantibodies against IL-17A, IL-17F, and IL-22 in patients with chronic mucocutaneous candidiasis and autoimmune polyendocrine syndrome type I. J. Exp. Med. 207, 291-297 (2010).
7. A. Puel, C. Picard, M. Lorrot, C. Pons, M. Chrabieh, L. Lorenzo, M. Mamani-Matsuda, E. Jouanguy, D. Gendrel, J.-L. Casanova, Recurrent staphylococcal cellulitis and subcutaneous abscesses in a child with autoantibodies against IL-6. J. Immunol. 180, 647-654 (2008).
8. B. E. Oftedal, A. Hellesen, M. M. Erichsen, E. Bratland, A. Vardi, J. Perheentupa, E. H. Kemp, T. Fiskerstrand, M. K. Viken, A. P. Weetman, S. J. Fleishman, S. Banka, W. G. Newman, W. a. C. Sewell, L. S. Sozaeva, T. Zayats, K. Haugarvoll, E. M. Orlova, J. Haavik, S. Johansson, P. M. Knappskog, K. Lsvis, A. S. B. Wolff, J. Abramson, E. S. Husebye, Dominant Mutations in the Autoimmune Regulator AIRE Are Associated with Common Organ-Specific Autoimmune Diseases. Immunity. 42, 1185-1196 (2015).
9. M. S. Anderson, J.-L. Casanova, More than Meets the Eye: Monogenic Autoimmunity Strikes Again. Immunity. 42, 986-988 (2015).
10. C.-H. Lin, C.-Y. Chi, H.-P. Shih, J.-Y. Ding, C.-C. Lo, S.-Y. Wang, C.-Y. Kuo, C.-F. Yeh, K.-H. Tu, S.-H. Liu, H.-K. Chen, C.-H. Ho, M.-W. Ho, C.-H. Lee, H.-C. Lai, C.-L. Ku, Identification of a major epitope by anti-interferon-γ autoantibodies in patients with mycobacterial disease. Nat. Med. 22, 994-1001 (2016).
11. A. Puel, J.-L. Casanova, Autoantibodies against cytokines: back to human genetics. Blood. 121, 1246-1247 (2013).
12. C.-Y. Chi, C.-C. Chu, J.-P. Liu, C.-H. Lin, M.-W. Ho, W.-J. Lo, P.-C. Lin, H.-J. Chen, C.-H. Chou, J.-Y. Feng, C.-P. Fung, Y.-P. Sher, C.-Y. Li, J.-H. Wang, C.-L. Ku, Anti-IFN-γ autoantibodies in adults with disseminated nontuberculous mycobacterial infections are associated with HLA-DRB1*16:02 and HLA-DQB1*05:02 and the reactivation of latent varicella-zoster virus infection. Blood. 121, 1357-1366 (2013).
13. A. Vallbracht, J. Treuner, B. Flehmig, K. E. Joester, D. Niethammer, Interferon-neutralizing antibodies in a patient treated with human fibroblast interferon. Nature. 289, 496-497 (1981).
14. null Pyhälä, Neutralizing antibodies against human leukocyte, lymphoblastoid and fibroblast interferons elicited by immunization with human leukocyte interferon. Acta Pathol Microbiol Scand C. 86C, 291-298 (1978).
15. K. Kisand, M. Link, A. S. B. Wolff, A. Meager, L. Tserel, T. Org, A. Murumägi, R. Uibo, N. Willcox, K. Trebusak Podkrajsek, T. Battelino, A. Lobell, O. Kämpe, K. Lima, A. Meloni, B. Ergun-Longmire, N. K. Maclaren, J. Perheentupa, K. J. E. Krohn, H. S. Scott, E. S. Husebye, P. Peterson, Interferon autoantibodies associated with AIRE deficiency decrease the expression of IFN-stimulated genes. Blood. 112, 2657-2666 (2008).
16. A. Meager, K. Visvalingam, P. Peterson, K. Möll, A. Murumägi, K. Krohn, P. Eskelin, J. Perheentupa, E. Husebye, Y. Kadota, N. Willcox, Anti-Interferon Autoantibodies in Autoimmune Polyendocrinopathy Syndrome Type 1. PLoS Med. 3 (2006), doi:10.1371/journal.pmed.0030289.
17. A. M. Morimoto, D. T. Flesher, J. Yang, K. Wolslegel, X. Wang, A. Brady, A. R. Abbas, V. Quarmby, E. Wakshull, B. Richardson, M. J. Townsend, T. W. Behrens, Association of endogenous anti-interferon-α autoantibodies with decreased interferon-pathway and disease activity in patients with systemic lupus erythematosus. Arthritis Rheum. 63, 2407-2415 (2011).
18. S. Gupta, I. P. Tatouli, L. B. Rosen, S. Hasni, I. Alevizos, Z. G. Manna, J. Rivera, C. Jiang, R. M. Siegel, S. M. Holland, H. M. Moutsopoulos, S. K. Browne, Distinct Functions of Autoantibodies Against Interferon in Systemic Lupus Erythematosus: A Comprehensive Analysis of Anticytokine Autoantibodies in Common Rheumatic Diseases. Arthritis & Rheumatology (Hoboken, N.J.). 68, 1677-1687 (2016).
19. S. Panem, I. J. Check, D. Henriksen, J. Vilcek, Antibodies to alpha-interferon in a patient with systemic lupus erythematosus. J. Immunol. 129, 1-3 (1982).
20. B. Pozzetto, K. E. Mogensen, M. G. Tovey, I. Gresser, Characteristics of autoantibodies to human interferon in a patient with varicella-zoster disease. J. Infect. Dis. 150, 707-713 (1984).
21. J. E. Walter, L. B. Rosen, K. Csomos, J. M. Rosenberg, D. Mathew, M. Keszei, B. Ujhazi, K. Chen, Y. N. Lee, I. Tirosh, K. Dobbs, W. Al-Herz, M. J. Cowan, J. Puck, J. J. Bleesing, M. S. Grimley, H. Malech, S. S. De Ravin, A. R. Gennery, R. S. Abraham, A. Y. Joshi, T. G. Boyce, M. J. Butte, K. C. Nadeau, I. Balboni, K. E. Sullivan, J. Akhter, M. Adeli, R. A. El-Feky, D. H. El-Ghoneimy, G. Dbaibo, R. Wakim, C. Azzari, P. Palma, C. Cancrini, K. Capuder, A. Condino-Neto, B. T. Costa-Carvalho, J. B. Oliveira, C. Roifman, D. Buchbinder, A. Kumanovics, J. L. Franco, T. Niehues, C. Schuetz, T. Kuijpers, C. Yee, J. Chou, M. J. Masaad, R. Geha, G. Uzel, R. Gelman, S. M. Holland, M. Recher, P. J. Utz, S. K. Browne, L. D. Notarangelo, Broad-spectrum antibodies against self-antigens and cytokines in RAG deficiency. J. Clin. Invest. 125, 4135-4148 (2015).
22. C. J. A. Duncan, S. M. B. Mohamad, D. F. Young, A. J. Skelton, T. R. Leahy, D. C. Munday, K. M. Butler, S. Morfopoulou, J. R. Brown, M. Hubank, J. Connell, P. J. Gavin, C. McMahon, E. Dempsey, N. E. Lynch, T. S. Jacques, M. Valappil, A. J. Cant, J. Breuer, K. R. Engelhardt, R. E. Randall, S. Hambleton, Human IFNAR2 deficiency: Lessons for antiviral immunity. Sci Transl Med. 7, 307ra154 (2015).
23. N. Hernandez, G. Bucciol, L. Moens, J. Le Pen, M. Shahrooei, E. Goudouris, A. Shirkani, M. Changi-Ashtiani, H. Rokni-Zadeh, E. H. Sayar, I. Reisli, A. Lefevre-Utile, D. Zijlmans, A. Jurado, R. Pholien, S. Drutman, S. Belkaya, A. Cobat, R. Boudewijns, D. Jochmans, J. Neyts, Y. Seeleuthner, L. Lorenzo-Diaz, C. Enemchukwu, I. Tietjen, H.-H. Hoffmann, M. Momenilandi, L. Poyhonen, M. M. Siqueira, S. M. B. de Lima, D. C. de Souza Matos, A. Homma, M. de L. S. Maia, T. A. da Costa Barros, P. M. N. de Oliveira, E. C. Mesquita, R. Gijsbers, S.-Y. Zhang, S. J. Seligman, L. Abel, P. Hertzog, N. Marr, R. de M. Martins, I. Meyts, Q. Zhang, M. R. MacDonald, C. M. Rice, J.-L. Casanova, E. Jouanguy, X. Bossuyt, Inherited IFNAR1 deficiency in otherwise healthy patients with adverse reaction to measles and yellow fever live vaccines. J. Exp. Med. (2019), doi:10.1084/jem.20182295.
24. S. Dupuis, E. Jouanguy, S. Al-Hajjar, C. Fieschi, I. Z. Al-Mohsen, S. Al-Jumaah, K. Yang, A. Chapgier, C. Eidenschenk, P. Eid, A. Al Ghonaium, H. Tufenkeji, H. Frayha, S. Al-Gazlan, H. Al-Rayes, R. D. Schreiber, I. Gresser, J.-L. Casanova, Impaired response to interferon-alpha/beta and lethal viral disease in human STAT1 deficiency. Nat. Genet. 33, 388-391 (2003).
25. S. Hambleton, S. Goodbourn, D. F. Young, P. Dickinson, S. M. B. Mohamad, M. Valappil, N. McGovern, A. J. Cant, S. J. Hackett, P. Ghazal, N. V. Morgan, R. E. Randall, STAT2 deficiency and susceptibility to viral illness in humans. Proc. Natd. Acad. Sci. U.S.A. 110, 3053-3058 (2013).
26. M. J. Ciancanelli, S. X. L. Huang, P. Luthra, H. Garner, Y. Itan, S. Volpi, F. G. Lafaille, C. Trouillet, M. Schmolke, R. A. Albrecht, E. Israelsson, H. K. Lim, M. Casadio, T. Hermesh, L. Lorenzo, L. W. Leung, V. Pedergnana, B. Boisson, S. Okada, C. Picard, B. Ringuier, F. Troussier, D. Chaussabel, L. Abel, I. Pellier, L. D. Notarangelo, A. Garcia-Sastre, C. F. Basler, F. Geissmann, S.-Y. Zhang, H.-W. Snoeck, J.-L. Casanova, Infectious disease. Life-threatening influenza and impaired interferon amplification in human IRF7 deficiency. Science. 348, 448-453 (2015).
27. N. Hernandez, I. Melki, H. Jing, T. Habib, S. S. Y. Huang, J. Danielson, T. Kula, S. Drutman, S. Belkaya, V. Rattina, L. Lorenzo-Diaz, A. Boulai, Y. Rose, N. Kitabayashi, M. P. Rodero, C. Dumaine, S. Blanche, M.-N. Lebras, M. C. Leung, L. S. Mathew, B. Boisson, S.-Y. Zhang, S. Boisson-Dupuis, S. Giliani, D. Chaussabel, L. D. Notarangelo, S. J. Elledge, M. J. Ciancanelli, L. Abel, Q. Zhang, N. Marr, Y. J. Crow, H. C. Su, J.-L. Casanova, Life-threatening influenza pneumonitis in a child with inherited IRF9 deficiency. J. Exp. Med. 215, 2567-2585 (2018).
28. H. K. Lim, S. X. L. Huang, J. Chen, G. Kerner, O. Gilliaux, P. Bastard, K. Dobbs, N. Hernandez, N. Goudin, M. L. Hasek, E. J. Garcia Reino, F. G. Lafaille, L. Lorenzo, P. Luthra, T. Kochetkov, B. Bigio, S. Boucherit, F. Rozenberg, C. Vedrinne, M. D. Keller, Y. Itan, A. Garcia-Sastre, M. Celard, J. S. Orange, M. J. Ciancanelli, I. Meyts, Q. Zhang, L. Abel, L. D. Notarangelo, H.-W. Snoeck, J.-L. Casanova, S.-Y. Zhang, Severe influenza pneumonitis in children with inherited TLR3 deficiency. J. Exp. Med. (2019), doi:10.1084/jem.20181621.
29. H. Jing, H. C. Su, New immunodeficiency syndromes that help us understand the IFN-mediated antiviral immune response. Curr. Opin. Pediatr. 31, 815-820 (2019).
30. G. Beccuti, L. Ghizzoni, V. Cambria, V. Codullo, P. Sacchi, E. Lovati, S. Mongodi, G. A. Iotti, F. Mojoli, A COVID-19 pneumonia case report of autoimmune polyendocrine syndrome type 1 in Lombardy, Italy: letter to the editor. J. Endocrinol. Invest. (2020), doi:10.1007/s40618-020-01323-4.
31. G. R. Burgio, F. Severi, R. Rossoni, R. Vaccaro, Autoantibodies in Down's syndrome. Lancet. 1, 497-498 (1966).
32. H. De Cauwer, A. Spaepen, Are patients with Down syndrome vulnerable to life-threatening COVID-19? Acta Neurol Belg (2020), doi:10.1007/s13760-020-01373-8.
33. J.-L. Casanova, H. C. Su, COVID Human Genetic Effort, A Global Effort to Define the Human Genetics of Protective Immunity to SARS-CoV-2 Infection. Cell. 181, 1194-1199 (2020).
34. J. Manry, G. Laval, E. Patin, S. Fornarino, Y. Itan, M. Fumagalli, M. Sironi, M. Tichit, C. Bouchier, J.-L. Casanova, L. B. Barreiro, L. Quintana-Murci, Evolutionary genetic dissection of human interferons. J. Exp. Med. 208, 2747-2759 (2011).
35. A. Mathian, S. Mouries-Martin, K. Dorgham, H. Devilliers, L. Barnabei, E. Ben Salah, F. Cohen-Aubart, L. Garrido Castillo, J. Haroche, M. Hie, M. Pineton de Chambrun, M. Miyara, D. Sterlin, M. Pha, D. Lê Thi Huong, F. Rieux-Laucat, F. Rozenberg, G. Gorochov, Z. Amoura, Monitoring Disease Activity in Systemic Lupus Erythematosus With Single-Molecule Array Digital Enzyme-Linked Immunosorbent Assay Quantification of Serum Interferon-α. Arthritis & Rheumatology (Hoboken, N.J.). 71, 756-765 (2019).
36. S. Gupta, I. P. Tatouli, L. B. Rosen, S. Hasni, I. Alevizos, Z. G. Manna, J. Rivera, C. Jiang, R. M. Siegel, S. M. Holland, H. M. Moutsopoulos, S. K. Browne, Distinct Functions of Anti-interferon Autoantibodies in Systemic Lupus Erythematosus: A Comprehensive Analysis of Anticytokine Autoantibodies in Common Rheumatologic Diseases. Arthritis Rheumatol. 68, 1677-1687 (2016).
37. S. Asgari, L. J. Schlapbach, S. Anchisi, C. Hammer, I. Bartha, T. Junier, G. Mottet-Osman, K. M. Posfay-Barbe, D. Longchamp, M. Stocker, S. Cordey, L. Kaiser, T. Riedel, T. Kenna, D. Long, A. Schibler, A. Telenti, C. Tapparel, P. J. McLaren, D. Garcin, J. Fellay, Severe viral respiratory infections in children with IFIH1 loss-of-function mutations. Proc. Natl. Acad. Sci. U.S.A. 114, 8342-8347 (2017).
38. I. T. Lamborn, H. Jing, Y. Zhang, S. B. Drutman, J. K. Abbott, S. Munir, S. Bade, H. M. Murdock, C. P. Santos, L. G. Brock, E. Masutani, E. Y. Fordjour, J. J. McElwee, J. D. Hughes, D. P. Nichols, A. Belkadi, A. J. Oler, C. S. Happel, H. F. Matthews, L. Abel, P. L. Collins, K. Subbarao, E. W. Gelfand, M. J. Ciancanelli, J.-L. Casanova, H. C. Su, Recurrent rhinovirus infections in a child with inherited MDA5 deficiency. J. Exp. Med. 214, 1949-1972 (2017).
39. S. Trouillet-Assant, S. Viel, A. Gaymard, S. Pons, J.-C. Richard, M. Perret, M. Villard, K. Brengel-Pesce, B. Lina, M. Mezidi, L. Bitker, A. Belot, COVID HCL Study group, Type I IFN immunoprofiling in COVID-19 patients. J. Allergy Clin. Immunol. (2020), doi:10.1016/j.jaci.2020.04.029.
40. J. Hadjadj, N. Yatim, L. Barnabei, A. Corneau, J. Boussier, H. Pere, B. Charbit, V. Bondet, C. Chenevier-Gobeaux, P. Breillat, N. Carlier, R. Gauzit, C. Morbieu, F. Pene, N. Marin, N. Roche, T.-A. Szwebel, N. Smith, S. Merkling, J.-M. Treluyer, D. Veyer, L. Mouthon, C. Blanc, P.-L. Tharaux, F. Rozenberg, A. Fischer, D. Duffy, F. Rieux-Laucat, S. Kerneis, B. Terrier, medRxiv, in press, doi:10.1101/2020.04.19.20068015.
41. J. Hadjadj, N. Yatim, L. Barnabei, A. Corneau, J. Boussier, N. Smith, H. Péré, B. Charbit, V. Bondet, C. Chenevier-Gobeaux, P. Breillat, N. Carlier, R. Gauzit, C. Morbieu, F. Pène, N. Marin, N. Roche, T.-A. Szwebel, S. H. Merkling, J.-M. Treluyer, D. Veyer, L. Mouthon, C. Blanc, P.-L. Tharaux, F. Rozenberg, A. Fischer, D. Duffy, F. Rieux-Laucat, S. Kerndis, B. Terrier, Impaired type I interferon activity and inflammatory responses in severe COVID-19 patients. Science (2020), doi:10.1126/science.abc6027.
42. A. T. Dilthey, P.-A. Gourraud, A. J. Mentzer, N. Cereb, Z. Iqbal, G. McVean, High-Accuracy HLA Type Inference from Whole-Genome Sequencing Data Using Population Reference Graphs. PLoS Comput. Biol. 12, e1005151 (2016).
43. A. Harris, J. Collins, D. Vetrie, C. Cole, M. Bobrow, X inactivation as a mechanism of selection against lethal alleles: further investigation of incontinentia pigmenti and X linked lymphoproliferative disease. J. Med. Genet. 29, 608-614 (1992).
44. H. Woffendin, T. Jakins, M. Jouet, H. Stewart, S. Landy, E. Haan, A. Harris, D. Donnai, A. Read, S. Kenwrick, X-inactivation and marker studies in three families with incontinentia pigmenti: implications for counselling and gene localisation. Clin. Genet. 55, 55-60 (1999).
45. M. Ehrenreich, M. M. Tarlow, E. Godlewska-Janusz, R. A. Schwartz, Incontinentia pigmenti (Bloch-Sulzberger syndrome): a systemic disorder. Cutis. 79, 355-362 (2007).
46. A. Smahi, G. Courtois, P. Vabres, S. Yamaoka, S. Heuertz, A. Munnich, A. Israël, N. S. Heiss, S. M. Klauck, P. Kioschis, S. Wiemann, A. Poustka, T. Esposito, T. Bardaro, F. Gianfrancesco, A. Ciccodicola, M. D'Urso, H. Woffendin, T. Jakins, D. Donnai, H. Stewart, S. J. Kenwrick, S. Aradhya, T. Yamagata, M. Levy, R. A. Lewis, D. L. Nelson, Genomic rearrangement in NEMO impairs NF-kappaB activation and is a cause of incontinentia pigmenti. The International Incontinentia Pigmenti (IP) Consortium. Nature. 405, 466-472 (2000).
47. F. Fusco, A. Pescatore, J. Steffann, J.-P. Bonnefont, J. De Oliveira, M. B. Lioi, M. V. Ursini, Clinical utility gene card: for incontinentia pigmenti. Eur. J. Hum. Genet. 27, 1894-1900 (2019).
48. D. Blanco-Melo, B. E. Nilsson-Payant, W.-C. Liu, S. Uhl, D. Hoagland, R. Msller, T. X. Jordan, K. Oishi, M. Panis, D. Sachs, T. T. Wang, R. E. Schwartz, J. K. Lim, R. A. Albrecht, B. R. tenOever, Imbalanced Host Response to SARS-CoV-2 Drives Development of COVID-19. Cell. 181, 1036-1045.e9 (2020).
49. X. Xia, Extreme genomic CpG deficiency in SARS-CoV-2 and evasion of host antiviral defense. Mol. Biol. Evol. (2020), doi:10.1093/molbev/msaa094.
50. Y. Xie, S. Cao, H. Dong, Q. Li, E. Chen, W. Zhang, L. Yang, S. Fu, R. Wang, Effect of regular intravenous immunoglobulin therapy on prognosis of severe pneumonia in patients with COVID-19. J. Infect. (2020), doi:10.1016/j.jinf.2020.03.044.
51. T. T. Wang, J. V. Ravetch, Functional diversification of IgGs through Fc glycosylation. J. Clin. Invest. 129, 3492-3498 (2019).
52. C. Ross, M. Svenson, M. B. Hansen, G. L. Vejlsgaard, K. Bendtzen, High avidity IFN-neutralizing antibodies in pharmaceutically prepared human IgG. J. Clin. Invest. 95, 1974-1978 (1995).
53. D. F. Robbiani, C. Gaebler, F. Muecksch, J. C. C. Lorenzi, Z. Wang, A. Cho, M. Agudelo, C. O. Barnes, A. Gazumyan, S. Finkin, T. Hägglöf, T. Y. Oliveira, C. Viant, A. Hurley, H.-H. Hoffmann, K. G. Millard, R. G. Kost, M. Cipolla, K. Gordon, F. Bianchini, S. T. Chen, V. Ramos, R. Patel, J. Dizon, I. Shimeliovich, P. Mendoza, H. Hartweger, L. Nogueira, M. Pack, J. Horowitz, F. Schmidt, Y. Weisblum, E. Michailidis, A. W. Ashbrook, E. Waltari, J. E. Pak, K. E. Huey-Tubman, N. Koranda, P. R. Hoffman, A. P. West, C. M. Rice, T. Hatziioannou, P. J. Bjorkman, P. D. Bieniasz, M. Caskey, M. C. Nussenzweig, Convergent antibody responses to SARS-CoV-2 in convalescent individuals. Nature (2020), doi:10.1038/s41586-020-2456-9.
54. K. Duan, B. Liu, C. Li, H. Zhang, T. Yu, J. Qu, M. Zhou, L. Chen, S. Meng, Y. Hu, C. Peng, M. Yuan, J. Huang, Z. Wang, J. Yu, X. Gao, D. Wang, X. Yu, L. Li, J. Zhang, X. Wu, B. Li, Y. Xu, W. Chen, Y. Peng, Y. Hu, L. Lin, X. Liu, S. Huang, Z. Zhou, L. Zhang, Y. Wang, Z. Zhang, K. Deng, Z. Xia, Q. Gong, W. Zhang, X. Zheng, Y. Liu, H. Yang, D. Zhou, D. Yu, J. Hou, Z. Shi, S. Chen, Z. Chen, X. Zhang, X. Yang, Effectiveness of convalescent plasma therapy in severe COVID-19 patients. Proc. Natl. Acad. Sci. U.S.A. 117, 9490-9496 (2020).
55. Y. Cheng, R. Wong, Y. O. Y. Soo, W. S. Wong, C. K. Lee, M. H. L. Ng, P. Chan, K. C. Wong, C. B. Leung, G. Cheng, Use of convalescent plasma therapy in SARS patients in Hong Kong. Eur. J. Clin. Microbiol. Infect. Dis. 24, 44-46 (2005).
56. D. M. Rissin, C. W. Kan, T. G. Campbell, S. C. Howes, D. R. Fournier, L. Song, T. Piech, P. P. Patel, L. Chang, A. J. Rivnak, E. P. Ferrell, J. D. Randall, G. K. Provuncher, D. R. Walt, D. C. Duffy, Single-molecule enzyme-linked immunosorbent assay detects serum proteins at subfemtomolar concentrations. Nat. Biotechnol. 28, 595-599 (2010).
57. P. Lebon, G. Ponsot, J. Aicardi, F. Goutieres, M. Arthuis, Early intrathecal synthesis of interferon in herpes encephalitis. Biomedicine. 31, 267-271 (1979).

### Example 3

### Autoantibodies to Type I IFNs can Underlie Adverse Reactions to Yellow Fever Live Attenuated Vaccine

Yellow fever virus (YFV) live attenuated vaccine can, in rare cases, cause life threatening disease, typically in patients with no previous history of severe viral illness. Autosomal recessive (AR) complete IFNAR1 deficiency was reported in one 14-year-old patient. Here, we studied seven other, previously healthy patients aged 13 to 80 years, with unexplained life-threatening YFV vaccine-associated disease. One 13-year-old patient had AR complete IFNAR2 deficiency. Three other patients vaccinated at the ages of 47, 57, and 64 years had high titers of circulating auto-Abs against at least 14 of the 17 individual type I IFN. These antibodies were recently shown to underlie at least 10% of cases of life threatening COVID-19 pneumonia. The auto-Abs were neutralizing in vitro, blocking the protective effect of IFN-α2 against YFV vaccine strains. AR IFNAR1 or IFNAR2 deficiency and neutralizing auto-Abs against type I IFNs thus accounted for more than half the cases of life-threatening YFV vaccine-associated disease studied here. Previously healthy subjects could be tested for both predispositions before anti-YFV vaccination.

Introduction

The 17D live-attenuated vaccine against yellow fever virus (YFV) was approved for use in humans by the World Health Organization in 1945. It has since been used to vaccinate more than 600 million people worldwide, with very high rates of seroconversion following the administration of a single dose, providing long-term protection (Monath, 2005; Monath et al., 2005). About half the vaccine recipients develop transient low-level viremia detectable four to six days after inoculation, a timing similar to that for viremia after wild-type YFV infection. All live attenuated YFV vaccines in current use are derivatives of the 17D strain and produced by amplification in embryonated chicken eggs. Although initially considered to be the world's safest live virus vaccine, rare cases of life-threatening disease following vaccination with YFV-17D were subsequently detected, from 2001 onward (Chan et al., 2001; Martin et al., 2001; Seligman, 2014; Vasconcelos et al., 2001). Systemic disease with clinical manifestations of organ dysfunction is often reported as yellow fever vaccine associated viscerotropic disease (YEL-AVD) (Seligman, 2014), and cases with neurological manifestations are referred to as yellow fever vaccine-associated neurological disease (YELAND). The prevalences of these conditions are about 0.3 and 0.8 per 100,000 vaccinees, respectively (Lindsey et al., 2016). However, prevalence estimates vary, ranging, for YELAVD, from 0-0.01 per 100,000 vaccinees in Africa, to 0.02-0.31 per 100,000 vaccinees in Brazil, and 0.35 per 100,000 vaccinees in USA, which is considered to be the most accurate estimate. The incidence of YEL-AND is estimated at 0.39 per 10′ administered vaccine doses (range 0.02-1.5) (Lecomte et al., 2020). Mortality rates vary depending on age, but approximatively two thirds of individuals die (Seligman, 2014). The rate of severe adverse events seems to increase with age, particularly after the age of 55 years, and is higher in men (Lindsey et al., 2016; Seligman, 2014). Women in their prime child-bearing years and patients with thymoma are also at risk (Seligman, 2014). Severe adverse reactions have also occurred in association with various autoimmune diseases, including systemic lupus erythematosus (SLE), Addison's disease, pernicious anemia, and myasthenia gravis, suggesting an immunological mechanism (de Menezes Martins et al., 2014; Seligman, 2014; Seligman and Casanova, 2016).

We previously described and provided an explanation for life-threatening disease following YFV-17D vaccination in 2019, with the discovery of autosomal recessive (AR), complete IFNAR1 deficiency in a 14-year-old Brazilian girl with no prior history of severe viral illness, who suffered from YEL-AVD (Hernandez et al., 2019). This study also highlighted the crucial role of type I interferons (IFNs) in controlling live attenuated YFV-17D. Four other patients with IFNAR1 deficiency have been reported: a nine-year-old child from Iran with measles-mumpsrubella (MMR) live vaccine-associated disease (Hernandez et al., 2019), a two-year-old child from Palestine with herpes simplex encephalitis (HSE) (Bastard et al., 2020a), and two adults, aged 26 and 38 years, from Saudi Arabia and Turkey, with life-threatening COVID-19 pneumonia (Zhang et al., 2020b). A case of YEL-AVD was also reported in a one-year-old patient with a suspected AR deficiency of IRF9, a component of the ISGF3 complex activated by both type I and III IFNs (Bravo Garcia-Morato et al., 2019). Another child, with severe influenza pneumonia, had proven AR IRF9 deficiency but was not vaccinated against yellow fever (Hernandez et al., 2018). Thus, inborn errors of type I IFN immunity can underlie life threatening disease following vaccination against YFV.

We studied seven other unrelated and previously healthy patients, currently aged 35 to 80 years, who had suffered from YEL-AVD (N=5) and/or YEL-AND (N=3) (Table 1) (Lecomte et al., 2020; Pulendran et al., 2008; Slesak et al., 2017). All had suffered life-threatening complications following vaccination with YFV-17D. In this report, we tested the hypothesis that some of these patients carry deleterious variants of genes of the type I IFN pathway, including IFNAR2, encoding the second chain of the type I IFN receptor (Duncan et al., 2015), JAK1 and TYK2, encoding the two kinases constitutively associated with the receptors (Eletto et al., 2016; Kreins et al., 2015; Lazear et al., 2019; Russell-Harde et al., 2000; Wilmes et al., 2015), and STAT1, STAT2, and IRF9, encoding the three components of ISGF-3 (Dupuis et al., 2003; Hambleton et al., 2013; Hernandez et al., 2018). We also tested the hypothesis that auto-Abs against type I IFNs may be causal for disease, as recently reported in 10% of patients suffering from severe COVID-19 (Bastard et al., 2020b; Zhang et al., 2020a)

Results

A Rare Homozygous Essential Splice Variant of IFNAR2 in One Patient

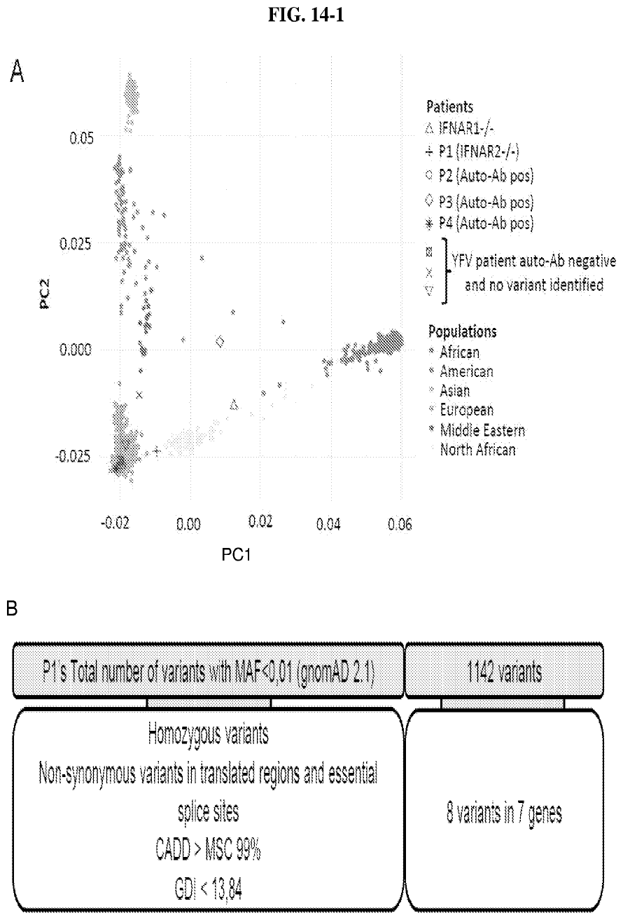

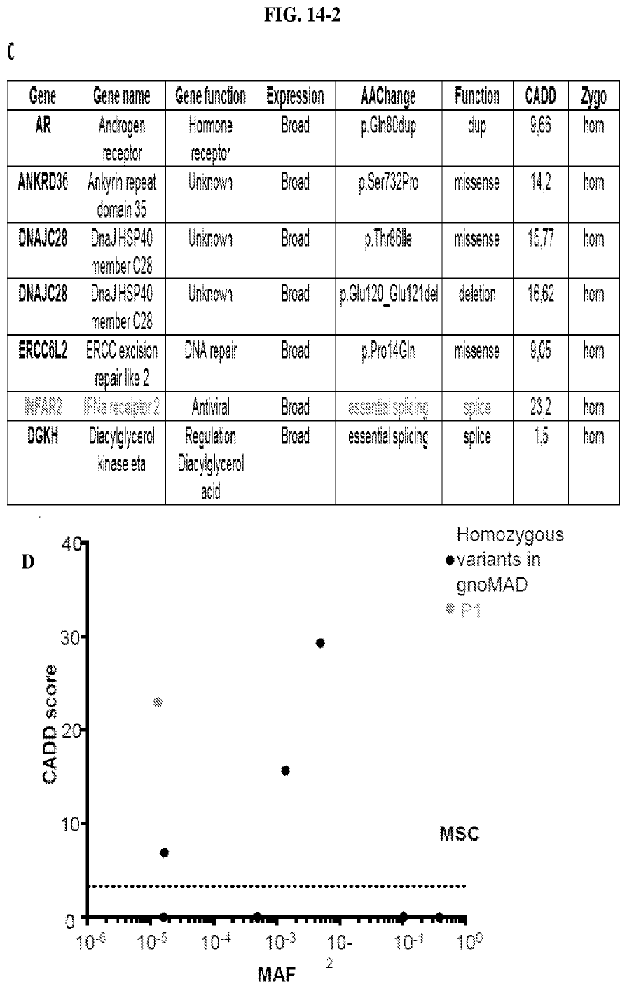

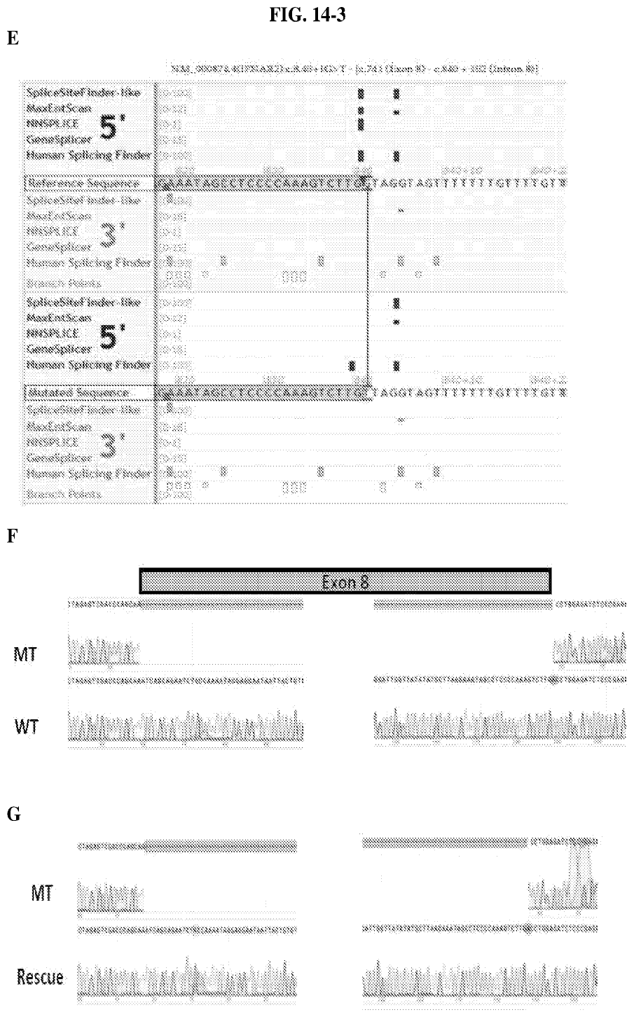

In addition to the patient with proven AR IFNAR1 deficiency (Hernandez et al., 2019), we studied seven patients who had experienced life-threatening disease following vaccination with the YFV-17D vaccine (Table 8). We performed whole-exome sequencing (WES) in all patients. Principal component analysis (PCA) confirmed that the patients were of different ancestries (FIG. 14A). We hypothesized that the patients developed YEL-AVD and/or YEL-AND because of monogenic inborn errors of immunity (IEI) compromising the cellular response to type I IFNs (IFNAR1, IFNAR2, STAT1, STAT2, IRF9 genes) (Bastard et al., 2020a; Duncan et al., 2015; Dupuis et al., 2003; Hambleton et al., 2013; Hernandez et al., 2019; Hernandez et al., 2018). We also considered possible defects of JAK1 and TYK2, although both encode proteins that are much more pleiotropic than the components of the ISGF3 complex (Eletto et al., 2016; Kreins et al., 2015). We tested this hypothesis by searching for homozygous single-nucleotide variants (SNVs) or large deletions (copy number variants, CNVs). We filtered out common variants (minor allele frequency [MAF]>0.01) and variants predicted to be benign (combined annotation-dependent depletion [CADD] score below the mutation significance cutoff [MSC] within the 99% confidence interval) (Itan et al., 2016; Kircher et al., 2014) (FIG. 14B). We also analyzed all genes underlying known inborn errors of immunity (IEI) (Bousfiha et al., 2020; Notarangelo et al., 2020; Tangye et al., 2020). We found that one patient (P1) carried a homozygous essential splicing variant of IFNAR2 (c.840+1G>T). P1 is a 35-year-old woman from Brazil, who suffered from YFV-AVD at the age of 13-years-old. She had no previous history of severe viral infections. Three days after vaccination with YFV-17D, P1 presented with fever and digestive symptoms. She was admitted to the hospital four days later for epistaxis, hepatitis, and hypotension. She recovered with supportive care. Her sister died from YEL-AVD at the age of 19 years, but no samples were available for this study (FIG. 11A). There were no rare variants in the other six candidate genes or in known IEI genes. The patient's homozygosity rate was 0.88% and there were only seven other homozygous rare non-synonymous variants in her exome, none of which was connected to anti-viral immunity (FIG. 14C). The IFNAR2 variant was confirmed by Sanger sequencing and was found to be present in the heterozygous state in both of P1's parents and in several other members of her family (FIG. 11B). This variant was present in the gnomADv3.1 public database but was extremely rare (MAF=1.3×10−5) and found only in the heterozygous state (FIG. 14D). Given the crucial role of human IFNAR2 in the type I IFN pathway (Duncan et al., 2015) and the previously described IFNAR1-deficient patient with YFV-AVD (Hernandez et al., 2019), we hypothesized that the homozygous essential splice site variant of IFNAR2 might underlie the pathogenesis of the adverse reaction to YFV 17D in P1.

TABLE 8
Clinical and epidemiological characteristics of the eight patients included in the
study with adverse events following YFV-17D vaccination.
IFN: interferon, YFV: yellow-fever virus, M: male, F: female; YEL-AVD:
yellow fever vaccine-associated viscerotropic disease; YEL-AND: yellow fever vaccineassociated
neurological disease. NT: non tested, yo: year old, CSF: cerebrospinal fluid
§ Coding regions of IFNAR1, IFNAR2, TYK2, JAK1, STATI, STAT2, and IRF9

| | Auto- | | | | | | |
|---|---|---|---|---|---|---|---|
| | Abr | Inborn | Country | | | | |
| | to | errors of | of origin | | | | YFV- |
| Case | type I | type I | and | | Age | Family | 17D |
| # | IFNs | IFNs^{§} | residence | Sex | (years) | history | disease |
| 1 | Negative | AR | Brazil | F | 16 | — | YEL- |
| | | IFNAR1 | | | | | AVD |
| | | deficiency | | | | | |
| 2 | Negative | AR | Brazil | F | 35 | Sister | YEL- |
| | | IFNAR1 | | | | died | AVD |
| | | deficiency | | | | from | |
| | | | | | | Yel- | |
| | | | | | | AVD at | |
| | | | | | | 19 yo | |
| 3 | Positive | Negative | Germany | M | 62 | — | YEL- |
| | | | | | | | AND/ |
| | | | | | | | YEL- |
| | | | | | | | AVD |
| 4 | Positive | Negative | Brazil | F | 50 | — | YEL- |
| | | | | | | | AVD |
| 5 | Positive | Negative | USA | M | 80 | — | YEL- |
| | | | | | | | AVD |
| 6 | Negative | Negative | Brazil | M | 25 | 2 siblings | YEL- |
| | | | | | | (brother, | AVD |
| | | | | | | aged 32 | |
| | | | | | | and sister, | |
| | | | | | | aged 32) | |
| | | | | | | died from | |
| | | | | | | YEL-AVD | |
| 7 | Negative | Negative | Brazil | M | 25 | Father: | YEL- |
| | | | | | | myasthenia | AVD |
| | | | | | | gravis | |
| 8 | Negative | Negative | Belgium | M | 56 | — | YEL- |
| | | | | | | | AVD |

| | | | | Other | | |
|---|---|---|---|---|---|---|
| | | | | severe | | |
| | Vaccin- | | | infections | Co- | |
| | ation | Clinical | | diseases | morbid- | |
| | year and | and | YFV- | and | ities | |
| Case | age | biological | 17D | age | (age of | |
| # | (years) | features | isolate | (years) | diagnosis) | Patient |
| 1 | 2017, | Fever, shock, | Blood | No | None | P2, |
| | 12 yo | renal and | | | | Hernandez |
| | | heparic | | | | et al., JEM |
| | | insufficiency, | | | | |
| | | pleural | | | | |
| | | effusion and | | | | |
| | | atelectans | | | | |
| 2 | 1999, | Fever, | Not | No | None | P1, this |
| | 13 yo | hypotension, | tested | | | report |
| | | bleeding, | | | | |
| | | hepatitis, | | | | |
| | | thrombopenia | | | | |
| 3 | 2016, | Fever, | Urine | Influenza B | None | P2, this |
| | 57 yo | meningo- | | pneumonia, | | report |
| | | encephalitis, | | 59 yo | | |
| | | hepatitis | | | | |
| 4 | 2017, | Fever, shock, | Blood | No | Systemic | P3, this |
| | 47 yo | renal | | | lupus | report |
| | | insufficiency, | | | erythema- | |
| | | heparic | | | tosus | |
| | | insufficiency, | | | (47 yo) | |
| | | thrombopenia | | | | |
| 5 | 2004, | Fever, shock, | Blood | No | None | P4, this |
| | 64 yo | renal | | | | report |
| | | insufficiency, | | | | |
| | | thrombopenia, | | | | |
| | | hepatitis | | | | |
| 6 | 2018, | Fever, nausea, | Blood | No | None | |
| | 28 yo | vomiting, | | | | |
| | | headache, | | | | |
| | | hepatitis | | | | |
| 7 | 2017, | Fever, | CSF | No | None | |
| | 22 yo | headache, | | | | |
| | | vomiting, | | | | |
| | | dizziness | | | | |
| 8 | 2020, | Fever, | CSF | No | None | |
| | 56 yo | headache, | | | | |
| | | cognitive | | | | |
| | | problems | | | | |

Autosomal Recessive Complete IFNAR2 Deficiency

The IFNAR2 variant was predicted in silico to alter splicing and to lead to the loss of exon 8 (FIG. 14E). We thus performed exon trapping on the patient's genomic DNA (gDNA) to test the impact of the variant. As predicted, a complete loss of exon 8 was observed in analyses of messenger RNA (mRNA) from the patient but not in analyses of mRNA from a healthy control (FIG. 11C, D and 4F). We also performed muragenesis on gDNA to restore the patient's variant to the WT form, which restored normal splicing (FIG. 14G). Human IFNAR2 is ubiquitously expressed transmembrane protein that constitutively binds JAK1 (Russell-Harde et. al., 2000; Wilmes et al., 2015). The loss of exon 8 is predicted to lead to a frameshift and a premature stop codon (p.Ser238Phefs*3) (FIG. 11E, F). We first studied the expression of the mutant (MT) IFNAR2 by plasmid-mediated overexpression in HEK293T cells. Following the transient transfection of cells with plasmids containing the WT or MT IFNAR2 cDNA (isoform c), similar levels of IFNAR2 mRNA were detected for the WT, MT and the previously published variant (p.E104fs110*) when using a probe spanning exons 1-2 (FIG. 11G). Western-blot (WB) analysis of these cell extracts with a C-terminal (C-ter) tag showed the protein to be absent, consistent with the loss of exon 8 leading to a frameshift without translation reinitiation (FIG. 11H). We then performed flow cytometry to analyze the cell surface expression of WT and MT IFNAR2 in transfected HEK293T cells. The MT protein was not detected on the cell surface (FIG. 11I). Finally, we used the WT or MT IFNAR2 cDNA to transfect IFNAR2 knock-out (KO) HEK293T cells, which we created by CRISPR/Cas9-mediated gene editing. Upon stimulation with IFN-α2 and transfection with a reporter gene, the cells expressing WT IFNAR2 displayed luciferase activity, unlike those expressing MT IFNAR2 (FIG. 11J). We did not test the response of the patient's cells to type I IFNs, but previous reports have indicated that the fibroblasts from patients with IFNAR2 deficiency are unresponsive to IFN-α2 and IFN-β (Bastard et al., 2020a; Duncan et al., 2015). P1 had AR complete IFNAR2 deficiency. Together with our previous report of a patient with AR IFNAR1 deficiency underlying YEL-AVD (Hernandez et al., 2019), these findings show that two of the eight patients in our cohort with severe adverse reactions to YFV-17D vaccination had an AR complete deficiency of one of the chains of the type I IFN receptor. They were surprisingly healthy prior to vaccination with YFV 17D at 12 and 13 years of age.

Auto-Abs Against IFN-α2 and IFN-ω in Three Unrelated Patients

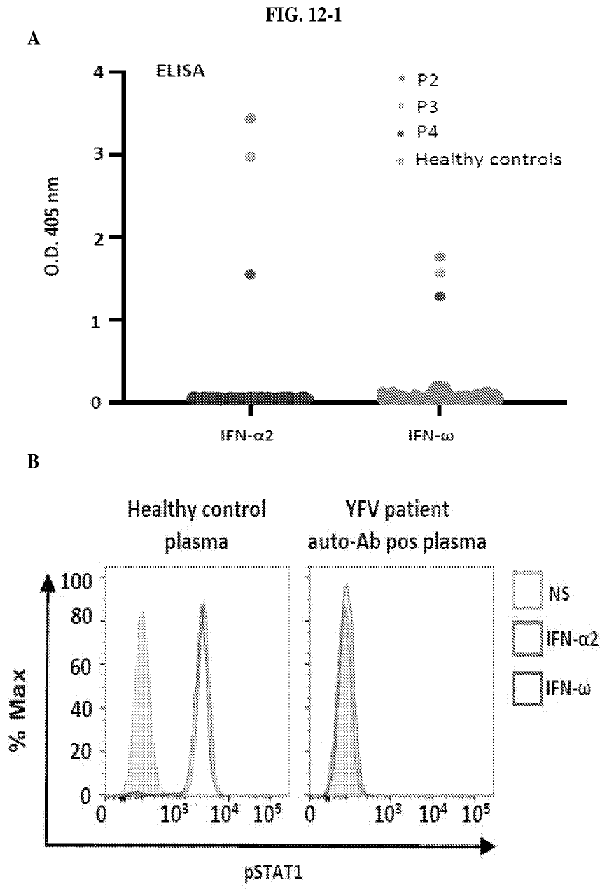

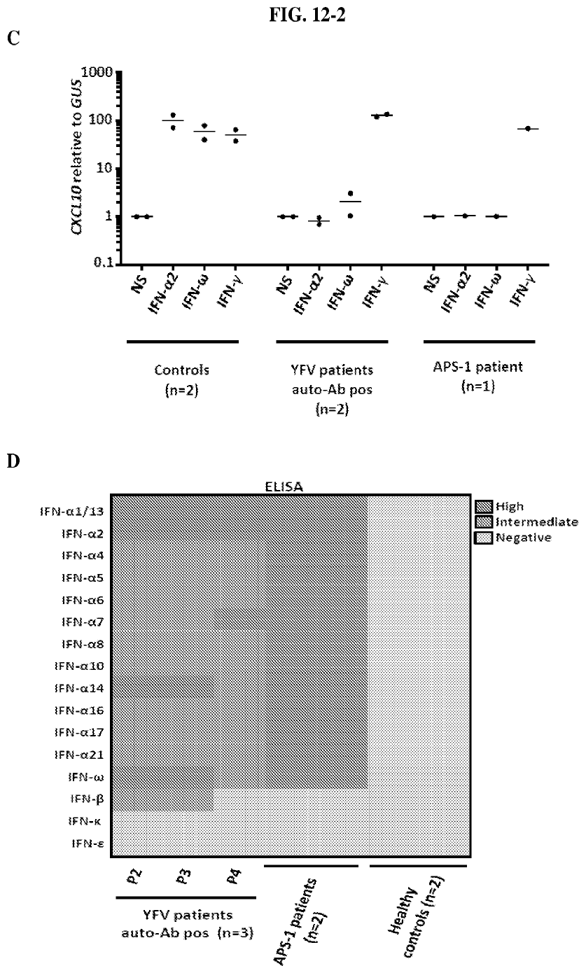

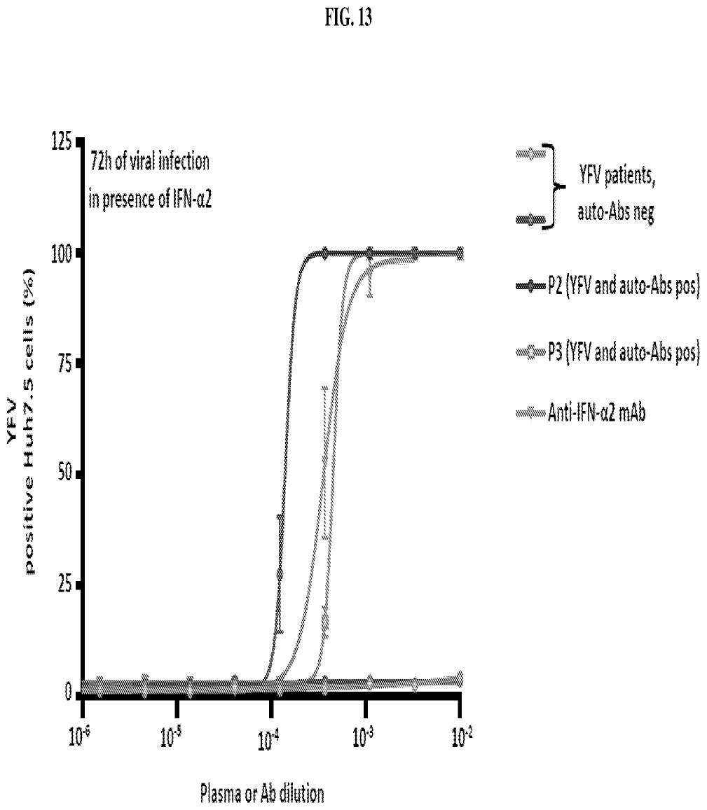

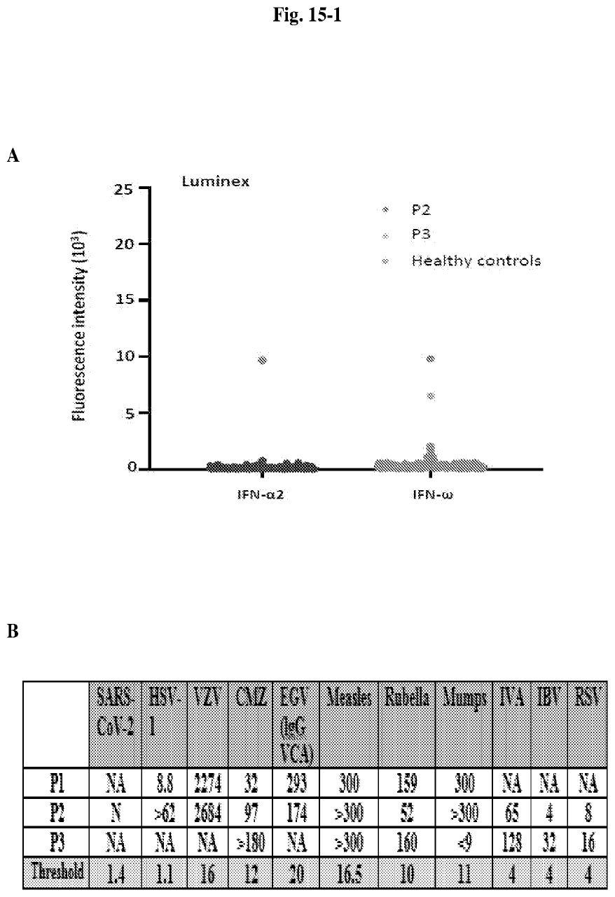

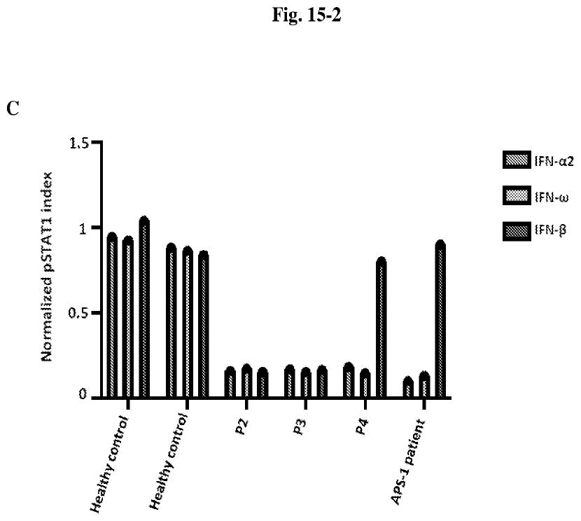

No non-synonymous variants of IFNAR1, IFNAR2, or of the five genes controlling the type I IFN response pathway (JAK1, TYK2, STAT1, STAT2, and IRF9) (Bastard et al., 2020a; Dupuis et al., 2003; Eletto et al., 2016; Gothe et al., 2020; Hambleton et al., 2013; Hernandez et al., 2018; Kreins et al., 2015; Watford and O'Shea, 2006) were found in the other six patients. We then hypothesized that these patients might have an autoimmune phenocopy of inborn errors of type I IFN immunity, as recently shown in patients with life-threatening COVID-19 pneumonia (Bastard et al., 2020b; Ku et al., 2020; Zhang et al., 2020a; Zhang et al., 2020b). We therefore searched for IgG auto-Abs against IFN-α2 and IFN-ω by ELISA and with a Luminex assay. We found high titers in three (P2, P3, and P4) of the six patients tested (FIGS. 12A and 15A). P2 is a 62-year-old man originating from and living in Germany, with no prior history of severe viral infections. At the age of 57 years, five days after YFV-17D vaccination, before traveling to Zanzibar, he developed neurological symptoms consistent with YEL-AND, and his liver enzyme levels increased (Slesak et al., 2017). He was also hospitalized two years later, at the age of 59 years, for influenza B infection with bilateral interstitial pneumonia. The blood sample positive for auto-Abs against type I IFNs was taken six months after YFV-17D disease. Patient 3 is a 50-year-old Brazilian woman with no history of severe viral infections. She presented with YFV-AVD following YFV-17D vaccination at the age of 47 years. Three days after vaccination, she reported fever, headache, and myalgia, which rapidly worsened. She was subsequently admitted to hospital with shock, acute renal and hepatic insufficiency, and thrombopenia. She was hospitalized in the intensive care unit (ICU) for two months. Of note, she reported a history of three months of polyarthralgia before hospitalization, and a history of miscarriage and preterm birth for her two pregnancies. She was diagnosed with systemic lupus erythematosus (SLE) during her hospital stay. Not coincidentally, women with SLE have been reported to be both at risk of adverse reactions to YFV-17D vaccination (Seligman, 2014) and to be prone to producing auto-Abs against type I IFN (Gupta et al., 2016; Howe and Leung, 2019; Mathian et al., 2019; Panem et al., 1982). The blood sample positive for auto-Abs against type I IFNs was taken when the patient was 49 years old, two years after YFV-17D disease. P4 is an 80-year-old man originating from and living in the United States, with no prior history of severe viral infections. At the age of 64, two days after YFV-17D vaccination, he developed fever and digestive symptoms. His condition quickly deteriorated, and he was hospitalized two days later for hypotension, skin rash, high fever, renal insufficiency, cytolysis and thrombopenia (Pulendran et al., 2008). The blood sample positive for auto-Abs was taken 16 days after the onset of YFV-17D disease. Interestingly, all patients tested (including P2 and P3) had been exposed to several common viruses (FIG. 15B) and P2 and P3 were positive for anti-nuclear auto-Abs (Table 9). Three of the eight patients with adverse reactions to YFV vaccination studied therefore had high titers of auto-Abs to type I IFNs.

Table 9 contains data on autoantibodies against other targets, used in clinical practice in two patients positive for auto-Abs against type I IFNs (P2 and P3).

TABLE 9
Autoantibodies against other targets, used in clinical practice
in two patients positive for anuto-Abs to type I IFNs (P2 and P3)

| | P2 | P3 |
|---|---|---|
| ANA by indirect immunofluores- | Positive | Positive |
| cence | | |
| Aspect | Homogeneous + | Speckled |
| | Nucleolar | |
| Titer ^{a} | 640 | 5120 |
| Anti-centromere antibodies | Negative | Negative |
| Anti-DNA | | |
| Indirect immunofluorescence | Negative | Negative |
| (Crithidia luciliae) | | |
| ELISA ^{b} | Positive | Negative |
| | (87 Ul/ml) | (18 Ul/ml) |
| Extractable Nuclear Antigen | | |
| Antibodies (ENA) Panel | | |
| by ELISA ^{c} | | |
| Anti-RNP | Negative | Positive (5.5) |
| Anti-Sm | Negative | Positive (2.9) |
| Anti-SSA/Ro | Negative | Negative |
| Anti-SSB/La | Negative | Negative |
| Anti-Scl70 | Negative | Negative |
| Anti-JO-1 | Negative | Negative |
| Extractable Nuclear Antigen | | |
| Antibodies (ENA) Panel | | |
| by DOT | | |
| Anti-RNP | Negative | Positive |
| Anti-Sm | Negative | Positive |
| Anti-SSA/Ro | Negative | Negative |
| Anti SSA/RO 52 kD | Negative | Positive |
| Anti-SSB/La | Negative | Negative |
| Anti-Scl70 | Negative | Negative |
| Anti-PM/Scl | Negative | Negative |
| Anti-JO1 | Negative | Negative |
| Anti-Centromere B | Negative | Negative |
| Anti-PCNA | Negative | Negative |
| Anti-Nucleosome | Negative | Negative |
| Anti-Histones | Negative | Positive |
| Anti-Ribosomes P | Negative | Negative |
| Anti-type 2 mitochondria | Negative | Negative |
| Anti-DFS 70 | Negative | Negative |

Anti-Sm: anti-Smith;
anti-RNP: anti-ribonucleoprotein;
Anti-Scl70: anti-topoisomerase I.
Anti-JO1: anti-Histidyl-tRNA synthetase;
Anti-DFS: anti-dense fine speckled;
Anti-PM/Scl: anti-poly-myositis/scleroderma;
Anti-PCNA: anti-proliferating cell nuclear antigen
^{a} Threshold: 1/80;
^{b} Threshold: 20 UI/ml;
^{c} Threshold: 1 (Optical density)

The Auto-Abs are Neutralizing and Recognize Most of the 17 Subtypes of Type I IFN

We then investigated whether these auto-Abs were neutralizing in vitro (i.e., if they could block the activity of recombinant type I IFNs in an experimental assay). We incubated PBMCs derived from a healthy control individual with 10% plasma from a healthy control or from the three patients, and stimulated the cells with IFN-α2 or IFN-ω). We found that the presence of plasma from the three patients completely abolished type I IFN signaling, as shown by analyses of pSTAT1 induction following treatment with IFN-α2 or IFN-ω) (FIGS. 12B and 15C). For P2 and P3, we also analyzed the induction of interferon-stimulated genes (ISG) after 2 h of stimulation with IFN-α2 or IFN-ω, using type II IFN (IFN-γ) as a control. The incubation of cells with plasma from either patient led to the abolition of ISG induction in response to either of the individual type I IFNs tested, but not in response to type II IFN. By contrast, induction in response to both type I and II IFNs remained normal in the presence of plasma from healthy control individuals (FIG. 12C). We tested the three patients for auto-Abs against the other 15 individual type I IFNs, by performing ELISA with recombinant proteins for the 17 individual type I IFNs. We found that P2, P3, and P4 had auto-Abs recognizing at least 14 subtypes (FIG. 12D), a finding similar to that for most of the patients with life threatening COVID-19 carrying auto-Abs against type I IFNs and autoimmune polyendocrine syndrome type I (APS-1) (see Example 2; Bastard et al., 2020b), although two patients (P2 and P3) also had neutralizing auto-Abs against IFN-β (FIG. 15C). The plasma samples from P2, P3 and P4 did not recognize IFN-κ or IFN-ε, at least in the experimental conditions tested. These two type I IFNs were not, however, sufficient to protect the patients from YFV-17D infection. Overall, we found that three patients presenting severe adverse reactions to YFV-17D vaccination had high titers of neutralizing auto-Abs against type I IFNs and that these auto-Abs recognized most of the 17 individual type I IFN subtypes.

The Auto-Abs Neutralize IFN-α2-Mediated Protection Against YFV 17D In Vitro

Finally, we investigated whether these auto-Abs blocking type I IFN in biochemical assays were also able to block type I IFN-mediated protection against YFV-17D infection in cells in vitro.

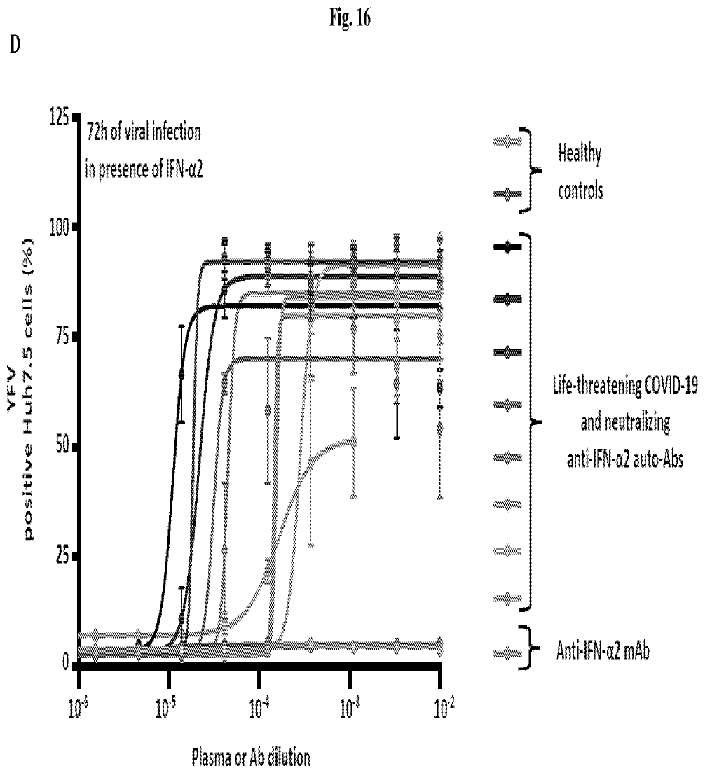

Plasma from P2 and P3, who had auto-Abs against type I IFNs, clearly blocked the ability of IFN-α2 to protect Huh-7.5 cells from infection with YFV-17D (FIG. 13). By contrast, plasma from two healthy donors without auto-Abs or from two other patients with severe YFV infection but without auto-Abs did not block the protective effect of IFN-α2 (FIG. 13). Furthermore, plasma from eight previously reported patients with life-threatening COVID-19 and auto-Abs against IFN-α2 also blocked the protective effect of IFN-α2 against YFV17D (FIG. 16), in addition to that against SARS-CoV-2 (Bastard et al., 2020b). Interestingly, the three patients reported here belong to two groups known to be at higher risk of displaying auto-Abs against type I IFNs: P2 and P4 are men over the age of 55 years (Bastard et al., 2020b; Seligman, 2014), and P3 is a 47 year old woman with SLE (Panem et al., 1982). Finally, we found that the prevalence of auto-Abs against type I IFNs in our cohort of patients with severe adverse events following YFV-17D vaccination and without known IEI was significantly higher than the estimated prevalence in the general population (Bastard et al., 2020b) (37.5% vs. 0.3%; Fisher's exact test, P=6.2×10−6). It is also probably not coincidental that the two patients with inherited IFNAR1 and IFNAR2 deficiency were younger, at 14 and 13 years, and did not display auto-Abs against type I IFNs. This suggests that the two mechanisms disrupting type I IFN immunity and resulting in YFV-17D disease are related but independent. This finding is reminiscent of our recent reports of inborn errors of type I IFNs and auto-Abs against type I IFNs in non-overlapping groups of patients with life-threatening COVID-19 pneumonia (Bastard et al., 2020b; Zhang et al., 2020a; Zhang et al., 2020b). Overall, these findings strongly suggest that the three patients reported here suffered adverse reactions to YFV-17D because of pre-existing neutralizing auto-Abs against type I IFNs, which targeted most of the individual subtypes of type I IFN, blocking their protective effect against YFV-17D.

Discussion

Impaired Type I IFN Immunity Underlies Yellow Fever Vaccine Adverse Events

Two members of our cohort of eight patients with YFV-AVD or YFV-AND have AR IFNAR1 or IFNAR2 deficiency, and another three have neutralizing auto-Abs against type I IFNs. These results suggest that at least half the patients with severe adverse reactions to YFV-17D have inadequate type I IFN immunity. This may be the case for the 78 reported patients with YFV-AVD or YEL-AND, including our five patients (de Menezes Martins et al., 2014; Hernandez et al., 2019; Lindsey et al., 2016; Seligman, 2014; Seligman and Casanova, 2016; Slesak et al., 2017). These findings unambiguously place human type I IFNs at the core of protective immunity to YFV-17D. They have important clinical implications. First, patients with known inborn errors of, or auto-Abs against type I IFN should not be vaccinated with YFV-17D. Second, patients with adverse reactions to YFV-17D should be tested for inborn errors of type I IFN immunity and for the presence of auto-Abs against type I IFNs. Third, both types of patients may benefit from treatment with recombinant IFN-β in the course of YFV-17D disease, provided that they do not have auto-Abs against IFN-β (such antibodies were present in two of the three patients reported here but were absent from 99 of 101 patients from a previous report on severe COVID-19) (Bastard et al., 2020b). Fourth, patients with autoimmune manifestations, including SLE and thymoma in particular, should be screened for auto-Abs against type I IFNs before vaccination with YFV-17D, as, perhaps, should men over the age of 55 years. Younger patients may nevertheless also be at risk of adverse reactions to YFV-17D vaccination due to pre-existing auto-Abs against type I IFNs, as suggested by the case of an eight-year-old patient homozygous for a hypomorphic RAG1 mutation who had defective adaptive immunity, multiple severe infectious diseases, auto-Abs against type I IFNs, and encephalitis after YFV-17D vaccination (Walter et al., 2015) (Table 2: JCI80477sdt1). Moreover, the youngest patient with life threatening COVID-19 pneumonia and auto-Abs against type I IFNs reported to date was 25 years old (Bastard et al., 2020b), younger than the two patients with inherited IFNAR1 deficiency and critical COVID-19, aged 26 and 38 years (Zhang et al., 2020b). Although difficult in some regions, screening for both inborn errors of type I IFN immunity and auto-Abs against type I IFNs could, therefore, be considered for patients of all ages before vaccination with YFV-17D. Moreover, patients with auto-Abs against type I IFNs are likely to be at risk of developing an adverse event if vaccinated with the newly developed vaccine against SARS-CoV-2 that uses the YFV live attenuated vaccine as carrier (Sanchez-Felipe et al., 2020). Sanchez-Felipe L et al 2020 describe the development of a candidate vaccine (YF-S0) for severe acute respiratory syndrome coronavirus 2 (SARS-CoV-2) that uses live-attenuated yellow fever 17D (YF17D) vaccine as a vector to express a noncleavable prefusion form of the SARS-CoV-2 spike antigen.

Pre-Existing Auto-Abs Against Type I IFNs can Underlie Different Viral Diseases

Our findings also have implications for the pathogenic role of auto-Abs against type I IFNs. Our previous study on life-threatening COVID-19 suggested that the auto-Abs against type I IFNs were causal of disease, rather than triggered by SARS-CoV-2 (see Example 2; Bastard et al., 2020b). Indeed, they were already present in blood samples from two of the 101 patients collected before the onset of COVID-19. Moreover, they were detected early in the course of SARS-CoV-2 infection in the other patients, making it highly unlikely that these auto-Abs of the IgG type with a high affinity for type I IFNs were triggered by infection. Finally, these antibodies are common in patients with two genetic diseases underlying severe COVID-19: APS-1 (Meager et al., 2006) and incontinentia pigmenti (Bastard et al., 2020b). The three patients with auto-Abs reported here add weight to this conclusion. Indeed, they developed YFV-17D disease within five days of vaccination. We did not test their blood for the presence of auto-Abs before vaccination, or even during YFV-17D disease, but the presence of these antibodies six months (P2), two years (P3) and 16 days (P4) later provides compelling evidence for an association with disease (Fisher's exact test, P=6.2×10−6), even without taking into account a previously reported patient whose YFV-17D disease may have been triggered by her many profound deficits of adaptive immunity other than auto-Abs against type I IFNs (Walter et al., 2015). Assuming that the three patients had auto-Abs at the time of hospital admission for YFV-17D disease, it would have been almost impossible for them to mount a neutralizing IgG response against a self-antigen so quickly. Furthermore, one of the patients (P3) was diagnosed with SLE during hospitalization and her SLE symptoms began three months before admission, consistent with the occurrence of such auto-Abs, as reported in about 5-10% of patients with SLE (Gupta et al., 2016; Mathian et al., 2019; Panem et al., 1982). The sex of the patient and age at the time of vaccination are similar to those in the other three known cases of SLE with YFV-17D disease (Seligman, 2014). Auto-Abs against type I IFNs can therefore underlie severe COVID-19 or YFV 17D disease. Intriguingly, children with APS-I and auto-Abs to type I IFNs have been vaccinated with MMR without any reported adverse reaction, despite the occurrence of MMR disease in several patients with inherited IFNAR1 or IFNAR2 deficiency (Bastard et al., 2020a; Duncan et al., 2015; Gothe et al., 2020; Hernandez et al., 2019). This may be due to the activity of IFN-β, IFN-κ, or IFN-ε in APS-I patients, whose auto-Abs typically neutralize the 13 individual IFN-α2 and IFN-ω (Bastard et al., 2020b; Meager et al., 2006). It is nevertheless tempting to speculate that other severe, unexplained viral illnesses, such as shingles and influenza, particularly, but not exclusively, in patients with autoimmune conditions or in elderly men, may be caused by auto-Abs against type I IFNs.

Methods

Patients and Study Approval

All cases of YEL-AVD and YEL-AND satisfied the Brighton Collaboration criteria (Gershman et al., 2012). The Brazilian patients were recruited by a collaboration with BioManguinhos/Fiocruz, as indicated in the acknowledgments. Additional patients were found from listings on ProMED (promedmail.org) and PubMed (pubmed.ncbi.nlm.nih.gov/). Written informed consent was obtained from patients in the country in which they were followed, in accordance with local regulations, and the study was approved by the institutional review boards of The Rockefeller University and Institut National de la Sante et de 1a Recherche Médicale (INSERM). Experiments were conducted in the United States of America and France, in accordance with local regulations and with the approval of the institutional review boards of The Rockefeller University and INSERM, respectively.

Whole-Exome Sequencing

Exome capture was performed with the SureSelect Human All Exon 50 Mb kit (Agilent Technologies). Paired-end sequencing was performed on a HiSeq 2000 (Illumina) generating 100-base reads. We aligned the sequences with the GRCh37 reference build of the human genome with the BWA (Li and Durbin, 2009). Downstream processing and variant calling were performed with the Genome Analysis Toolkit, SAMtools, and Picard (Li et al., 2009; McKenna et al., 2010). Substitution and InDel calls were made with GATK Unified Genotyper. All variants were annotated with an annotation software system developed inhouse (Adzhubei et al., 2010; Kircher et al., 2014; Ng and Henikoff, 2001). The patients reported here did not consent to the deposition of their genomic data.

Statistical Analysis

Comparison of proportions were performed using a Fisher exact test, as implemented in R (cran.r-project.org/). PCA was performed with Plink v1.9 software on wholeexome and whole-genome sequencing data with the 1000 Cenomes (1 kG) Project phase 3 public data-base as a reference.

Cells

Peripheral blood mononuclear cells (PBMCs) were isolated by Ficoll-Paque density gradient (GE Life Science, USA) centrifugation. Primary fibroblasts, SV40-immortalized dermal fibroblasts, HEK293T kidney epithelial cells (H. sapiens), VeroE6 kidney epithelial cells (Chlorocebus sabaeus) and Huh-7.5 hepatoma cells (H. sapiens) were maintained in Dulbecco's modified Eagle medium (DMEM, Thermo Fisher Scientific) supplemented with 10% fetal bovine serum (FBS) at 37° C. under an atmosphere containing 5% CO2. EBV-B cells were maintained in Roswell Park Memorial Institute medium (RPM1, Thermo Fisher Scientific) supplemented with 10% FBS. All cells tested negative for mycoplasma contamination.

Exon Trapping

DNA segments encompassing the IFNAR2 exon 8 region were amplified from genomic DNA and inserted into the pSPL3 vector, between the EcoRI and BamHI sites. Wildtype, mutant (IFNAR2 c.840+1G>T) or mutagenesis rescue plasmids were used to transfect COS-7 cells. After 24 h, total RNA was extracted and reverse-transcribed. The IFNAR2 splicing products were amplified with flanking HIV-TAT sequences from the pSPL3 vector and ligated into the pCR4-TOPO vector (Invitrogen). Stellar cells (Takara) were transformed with the resulting plasmids. Colony PCR and sequencing with primers binding to the flanking HIV-TAT sequences of pSPL3 were performed to determine the splicing products produced by the WT and mutated alleles.

Plasmids

A plasmid containing the cDNA of IFNAR2 was used (generously provided by Sandra Pellegrini) and site-directed mutagenesis was performed to obtain the indicated mutant constructs.

IFNAR2 Overexpression by Plasmid Transfection and the Generation of Stably Reconstituted Cell Lines

The IFNAR2 plasmid was used to transfect HEK293T cells by incubation for 48 h, in the presence of X-tremeGene 9 transfection reagent (Sigma Aldrich). We used 1 μg of plasmid to transfect 0.5×106 cells.

Western Blotting

HEK293T cells were transfected for 36 h with WT or MT IFNAR2. Cells were lysed in NP40 lysis buffer (280 mM NaCl, 50 mM Tris pH 8, 0.2 mM EDTA, 2 mM EGTA, 10% glycerol, 0.5% NP40) supplemented with 1 mM DTT, PhosSTOP (Roche, Mannheim, Germany) and complete protease inhibitor cocktail (Roche, Mannheim, Germany). The protein lysate was subjected to SDS-PAGE and the bands obtained were transferred to a nitrocellulose membrane. For detection of the protein overproduced following transfection, we used an HRP-conjugated anti-V5 antibody purchased from commercial suppliers. An anti-GAPDH (Santa Cruz) antibody was used as a loading control. The membrane was incubated overnight at 4° C. with the primary antibodies. SuperSignal West Pico chemiluminescent substrate (Thermo Fisher Scientific) was used to visualize HRP activity after incubation with secondary antibody, and this signal was detected with an Amersham Imager 600 (GE Life Sciences).

Flow Cytometry

The cell surface expression of IFNAR2 was assessed with a PE-conjugated mouse anti-IFNAR2 (#21385-3PBL Assay Science, Piscataway, N.J., USA) antibody. Cells were stained and then washed twice with PBS and analyzed by flow cytometry. Data were acquired on a Gallios flow cytometer.

Generation of IFNAR2-Deficient HEK293T Cells

IFNAR2-deficient HEK293T cells were generated with the CRISPR/Cas9 system. Guide RNAs were designed with the Benchling design tool, and inserted into lentiCRISPR v2, which was a gift from Feng Zhang (Addgene plasmid #52961). The three guide RNAs were designed to bind and cut at different places in the IFNAR2 gene, one in exon 3 (FOR: CACCGCATATGAAATACCAAACACG (SEQ ID NO:1); REV: AAACCGTGTTTGGTATTTCATATGC (SEQ ID NO:2)), one in exon 4 (FOR: CACCGCATTGCTGTATACAATCATG (SEQ ID NO:3); REV: AAACCATGATTGTATACAGCAATGC (SEQ ID NO:4)), one in exon 5 (FOR: CACCGTGAGTGGAGAAGCACACACG (SEQ ID NO:5); REV: AAACCGTGTGTGCTTCTCCACTCAC (SEQ ID NO:6)). Using X-tremeGENE 9 DNA Transfection Reagent (Roche), we transiently transfected WT HEK293T cells with the resulting plasmids and cultured them for seven days before sorting IFNAR2-deficient cells by flow cytometry after staining with an antibody against IFNAR2 (Miltenyi Biotec, REA124). The three resulting cell lines were subsequently tested to check for a lack of IFNAR2 expression at the cell surface and were used in the luciferase assays.

Luciferase Reporter Assays

IFNAR2′ HEK293T cells generated by CRISPR/Cas9 system were transfected with the indicated expression plasmids, firefly luciferase plasmids under the control of WT or Mut IFNAR2, or human ISRE promoters in the pGL4.45 backbone, and a constitutively expressing Renilla luciferase plasmid for normalization (pRL-SV40). Cells were transfected in the presence of the X-tremeGene 9 transfection reagent (Sigma Aldrich) for 36 hours. Luciferase levels were measured with the Dual-Glo reagent, according to the manufacturer's protocol (Promega). Firefly luciferase values were normalized against Renilla luciferase values, and fold induction is shown relative to controls transfected with empty plasmids.

Quantitative RT-PCR

RNA was isolated from PBMCs, or from plasmid-transfected or untransfected HEK293T cells, with a kit, according to the manufacturer's protocol (Zymo Research, Irvine, Calif.), and treated with DNase (Zymo Research, Irvine, Calif.). Reverse transcription was performed with random hexamers and the Superscript III reverse-strand synthesis kit, according to the manufacturer's instructions (Thermo Fisher Scientific, Springfield Township, N.J.). Quantitative real-time PCR (qPCR) was performed with Applied Biosystems Taqman assays for IFNAR2, and the f glucuronidase (GUS) housekeeping gene for normalization. Results are expressed according to the ΔΔCt method, as described by the kit manufacturer.

Detection of Anti-Cytokine Autoantibodies in a Multiplex Particle-Based Assay

Serum/plasma samples were screened for autoantibodies against IFN-α2 and IFN-ω targets in a multiplex particle-based assay, in which magnetic beads with differential fluorescence were covalently coupled to recombinant human proteins (2.5 μg/reaction). Beads were combined and incubated with 1:100 diluted serum/plasma samples for 30 minutes. Each sample was tested once. The beads were then washed and incubated with PE-labeled goat anti-human IgG (1 μg/mL) for 30 minutes. They were washed again and used in a multiplex assay run on a BioPlex X200 instrument. Patients with a fluorescence intensity (FI) >1500 for IFN-α2 or IFN-β, or >1000 for IFN-ω were tested for blocking activity.

Enzyme-Linked Immunosorbent Assays (ELISA) for Anti-Cytokine Autoantibodies

ELISA was performed as previously described (Bastard et al., 2020b). In brief, 96-well ELISA plates (MaxiSorp; Thermo Fisher Scientific) were coated by incubation overnight at 4° C. with 2 g/mL rhIFN-α, and rhIFN-ω (R&D Systems). Plates were then washed (PBS/0.005% Tween), blocked by incubation with 5% nonfat milk powder in the same buffer, washed, and incubated with 1:50 dilutions of plasma from the patients or controls for 2 h at room temperature (or with specific mAbs as positive controls). Each sample was tested once. Plates were thoroughly washed. Horseradish peroxidase (HRP)-conjugated Fc-specific IgG fractions from polyclonal goat antiserum against human IgG or IgA (Nordic Immunological Laboratories) were added to a final concentration of 2 g/mL. Plates were incubated for 1 h at room temperature and washed. Substrate was added and the optical density (O.D.) was measured. A similar protocol was used to test for antibodies against 12 subtypes of IFN-α, except that the plates were coated with cytokines from PBL Assay Science (catalog #11002-1).

Clinical Screening for Other Autoantibodies

The assay for ANA was performed using an indirect immunofluorescence on Hep-2 cells (Novalite ref #704320, Inova Diagnostics San Diego, Calif., distributed by Werfen, Le Prd-Saint-Gervais France). Dilutions of 1:80 were performed with phosphate-buffered saline for screening test. In brief, L of each diluted serum was incubated on one well with fixed Hep-2 cells. After the cells were incubated and washed, they were incubated with DAPI-IgG conjugated antibody. The results were analyzed using a Nikon Eclipse fluorescence microscope with a magnification ×400. Extractable Nuclear Antigen (ENA) Antibodies were detected by ELISA (RNP, Sm, SSA/Ro, SSB/1a, Sc170 and JO-1) using the ANA 8 screen Kit (Euroimmun Bussy-Saint-Martin, France). Immunoblotting was performed for a larger ENA panel detection (RNP, Sm, SSA/Ro, SSA/Ro 52 kD, SSB/1a, Sc170, JO-1, Centrome B, PCNA, Nucleosome, Histones, Ribosomes P, Type 2 mitochondria, DFS 70) using the ANA Profil 3 Dot (Euroimmun Bussy-Saint-Martin, France). Anti-native DNA detection was performed by indirect immunofluorescence on the flagellate organism Crithidia luciliae using the KIT Theradiag (Croissy-Beaubourg, France) and ELISA for antibodies quantification using the anti-dsDNA IgG KIT on ETI-MAX 3000 Equipment from Diasorin (Antony, France).

Functional Evaluation of Anti-Cytokine Autoantibodies

The blocking activity of anti-IFN-α and anti-IFN-ω autoantibodies was determined by assessing STAT1 phosphorylation in healthy control cells following stimulation with the appropriate cytokines in the presence of 10% serum/plasma from a healthy control or a patient. Surface-stained healthy control PBMCs (350,000/reaction) were cultured in serumfree RPMI medium supplemented with 10% healthy control or patient serum/plasma and were either left unstimulated or were stimulated with IFN-α or IFN-ω (10 ng/mL) for 15 minutes at 37° C. Each sample was tested once. Cells were fixed, permeabilized, and stained for intranuclear phospho-STAT1 (Y701). Cells were acquired on a BD LSRFortessa cytometer with gating on CD14+ monocytes and analyzed with FlowJo software.

YFV-17D Experiment

The generation of virus stocks for the YFV 17D reporter virus expressing the Venus fluorescent protein (YFV-Venus) (derived from YF17D-5′C25Venus2AUbi) has been described elsewhere (Yi et al., 2011). Virus stock titers were determined by standard plaque assays on Huh-7.5 cells. YFV-Venus experiments were performed as follows: Huh-7.5 cells were used to seed 96-well plates at a density of 5×103 cells/well, with triplicate wells for each sample. The following day, serial three-fold dilutions of plasma samples or a commercial anti-IFN-α2 antibody (R&D systems, cat. #21100-1) were incubated with 20 pM recombinant IFN-α2 (R&D systems, cat. #11101-2) for 1 h at 37° C. (starting dilution: plasma samples=1/100 and anti-IFN-α2 antibody=1/1,000). Following this incubation period, the cell culture medium was removed from the wells of the 96-well plate by aspiration and replaced with the plasma/antibody-IFN-α2 mixture. The plates were incubated overnight, the plasma/antibody-IFN-α2 mixture was removed and the plates were washed with PBS to remove any anti-YFV-neutralizing antibodies present. Fresh medium was then added to the wells. Cells were then infected with YFV-Venus at a MOI of 0.05 PFU/cell, by directly dispensing the inoculum in the wells. After three days, the cells were fixed in formaldehyde, stained with DAPI, and imaged and analyzed as previously described (Bastard et al., 2020b).

### REFERENCES

Adzhubei, I. A., S. Schmidt, L. Peshkin, V. E. Ramensky, A. Gerasimova, P. Bork, A. S. Kondrashov, and S. R. Sunyaev. 2010. A method and server for predicting damaging missense mutations. Nat Methods 7:248-249.
Bastard, P., J. Manry, J. Chen, J. Rosain, Y. Seeleuthner, O. AbuZaitun, L. Lorenzo, T. Khan, M. Hasek, N. Hernandez, B. Bigio, P. Zhang, R. Levy, S. Shrot, E. J. Garcia Reino, Y. S. Lee, S. Boucherit, M. Aubart, R. Gijsbers, V. Beziat, Z. Li, S. Pellegrini, I. Meyts, F. Rozenberg, N. Marr, B. Boisson, A. Cobat, J. Bustamante, Q. Zhang, E. Jouanguy, L. Abel, R. Somech, J. L. Casanova, and S. Y. Zhang. 2020a. Herpes simplex encephalitis in a patient with a distinctive form of inherited IFNAR1 deficiency. J Clin Invest 131(1):e139980. doi: 10.1172/JCI139980.
Bastard, P., L. B. Rosen, Q. Zhang, E. Michailidis, H. H. Hoffmann, Y. Zhang, K. Dorgham, Q. Philippot, J. Rosain, V. Beziat, J. Manry, E. Shaw, L. Haljasmagi, P. Peterson, L. Lorenzo, L. Bizien, S. Trouillet-Assant, K. Dobbs, A. A. de Jesus, A. Belot, A. Kallaste, E. Catherinot, Y. Tandjaoui-Lambiotte, J. Le Pen, G. Kerner, B. Bigio, Y. Seeleuthner, R. Yang, A. Bolze, A. N. Spaan, O. M. Delmonte, M. S. Abers, A. Aiuti, G. Casari, V. Lampasona, L. Piemonti, F. Ciceri, K. Bilguvar, R. P. Lifton, M. Vasse, D. M. Smadja, M. Migaud, J. Hadjadj, B. Terrier, D. Duffy, L. Quintana-Murci, D. van de Beek, L. Roussel, D. C. Vinh, S. G. Tangye, F. Haerynck, D. Dalmau, J. Martinez-Picado, P. Brodin, M. C. Nussenzweig, S. Boisson-Dupuis, C. Rodriguez-Gallego, G. Vogt, T. H. Mogensen, A. J. Oler, J. Gu, P. D. Burbelo, J. Cohen, A. Biondi, L. R. Bettini, M. D'Angio, P. Bonfanti, P. Rossignol, J. Mayaux, F. Rieux-Laucat, E. S. Husebye, F. Fusco, M. V. Ursini, L. Imberti, A. Sottini, S. Paghera, E. Quiros-Roldan, C. Rossi, R. Castagnoli, D. Montagna, A. Licari, G. L. Marseglia, X. Duval, J. Ghosn, H. Lab, N.-U. I. R.t.C. Group, C. Clinicians, C.-S. Clinicians, C. G. Imagine, C. C. S. G. French, C. Milieu Interieur, V. C. C. Co, U. M. C. C.-B. Amsterdam, C. H. G. Effort, J. S. Tsang, R. Goldbach-Mansky, K. Kisand, M. S. Lionakis, A. Puel, S. Y. Zhang, S. M. Holland, G. Gorochov, E. Jouanguy, C. M. Rice, A. Cobat, L. D. Notarangelo, L. Abel, H. C. Su, and J. L. Casanova. 2020b. Auto-antibodies against type I IFNs in patients with life-threatening COVID-19. Science 23; 370(6515):eabd4585. doi: 10.1126/science.abd4585.
Bousfiha, A., L. Jeddane, C. Picard, W. Al-Herz, F. Ailal, T. Chatila, C. Cunningham-Rundles, A. Etzioni, J. L. Franco, S. M. Holland, C. Klein, T. Morio, H. D. Ochs, E. Oksenhendler, J. Puck, T. R. Torgerson, J. L. Casanova, K. E. Sullivan, and S. G. Tangye. 2020. Human Inborn Errors of Immunity: 2019 Update of the IUIS Phenotypical Classification. J Clin Immunol 40:66-81.
Bravo Garcia-Morato, M., A. Calvo Apalategi, L. Y. Bravo-Gallego, A. Blazquez Moreno, M. Simon-Fuentes, J. V. Garmendia, A. Mendez Echevarria, T. Del Rosal Rabes, A. Dominguez-Soto, E. Lopez-Granados, H. T. Reyburn, and R. Rodriguez Pena. 2019. Impaired control of multiple viral infections in a family with complete IRF9 deficiency. J Allergy Clin Immunol 144:309-312 e310.
Chan, R. C., D. J. Penney, D. Little, I. W. Carter, J. A. Roberts, and W. D. Rawlinson. 2001. Hepatitis and death following vaccination with 17D-204 yellow fever vaccine. Lancet 358:121-122.
de Menezes Martins, R., A. L. Pavao, P. M. de Oliveira, P. R. dos Santos, S. M. Carvalho, R. Mohrdieck, A. R. Fernandes, H. K. Sato, P. M. de Figueiredo, R. von Doellinger Vdos, L. Leal Mda, A. Homma, and L. Maia Mde. 2014. Adverse events following yellow fever immunization: Report and analysis of 67 neurological cases in Brazil. Vaccine 32:6676-6682.
Duncan, C. J., S. M. Mohamad, D. F. Young, A. J. Skelton, T. R. Leahy, D. C. Munday, K. M. Butler, S. Morfopoulou, J. R. Brown, M. Hubank, J. Connell, P. J. Gavin, C. McMahon, E. Dempsey, N. E. Lynch, T. S. Jacques, M. Valappil, A. J. Cant, J. Breuer, K. R. Engelhardt, R. E. Randall, and S. Hambleton. 2015. Human IFNAR2 deficiency: Lessons for antiviral immunity. Sci Transl Med 7:307ra154.
Dupuis, S., E. Jouanguy, S. Al-Hajjar, C. Fieschi, I. Z. Al-Mohsen, S. Al-Jumaah, K. Yang, A. Chapgier, C. Eidenschenk, P. Eid, A. Al Ghonaium, H. Tufenkeji, H. Frayha, S. Al-Gazlan, H. Al-Rayes, R. D. Schreiber, I. Gresser, and J. L. Casanova. 2003. Impaired response to interferon-alpha/beta and lethal viral disease in human STAT1 deficiency. Nat Genet 33:388-391.
Eletto, D., S. O. Burns, I. Angulo, V. Plagnol, K. C. Gilmour, F. Henriquez, J. Curtis, M. Gaspar, K. Nowak, V. Daza-Cajigal, D. Kumararatne, R. Doffinger, A. J. Thrasher, and S. Nejentsev. 2016. Biallelic JAK1 mutations in immunodeficient patient with mycobacterial infection. Nat Commun 7:13992.
Gershman, M. D., J. E. Staples, A. D. Bentsi-Enchill, J. G. Breugelmans, G. S. Brito, L. A. Camacho, P. Cottin, C. Domingo, A. Durbin, J. Gascon, F. Guenaneche, E. B. Hayes, Z. Jelenik, A. Khromava, M. Martins Rde, M. M. Wilson, N. Massy, A. Nasidi, M. Niedrig, A. Sherwat, T. Tsai, A. Vilella, M. E. Wilson, K. S. Kohl, and G. Brighton Collaboration Viscerotropic Disease Working. 2012. Viscerotropic disease: case definition and guidelines for collection, analysis, and presentation of immunization safety data. Vaccine 30:5038-5058.
Gothe, F., C. F. Hatton, L. Truong, Z. Klimova, V. Kanderova, M. Fejtkova, A. Grainger, V. Bigley, J. Perthen, D. Mitra, A. Janda, E. Fronkova, D. Moravcikova, S. Hambleton, and C. J. A. Duncan. 2020. A novel case of homozygous IFNAR1 deficiency with haemophagocytic lymphohistiocytosis. Clin Infect Dis ciaa1790.doi: 10.1093/cid/ciaa1790.
Gupta, S., I. P. Tatouli, L. B. Rosen, S. Hasni, I. Alevizos, Z. G. Manna, J. Rivera, C. Jiang, R. M. Siegel, S. M. Holland, H. M. Moutsopoulos, and S. K. Browne. 2016. Distinct Functions of Autoantibodies Against Interferon in Systemic Lupus Erythematosus: A Comprehensive Analysis of Anticytokine Autoantibodies in Common Rheumatic Diseases. Arthritis Rheumatol 68:1677-1687.
Hambleton, S., S. Goodbourn, D. F. Young, P. Dickinson, S. M. Mohamad, M. Valappil, N. McGovern, A. J. Cant, S. J. Hackett, P. Ghazal, N. V. Morgan, and R. E. Randall. 2013. STAT2 deficiency and susceptibility to viral illness in humans. Proc Natl Acad Sci USA 110:3053-3058.
Hernandez, N., G. Bucciol, L. Moens, J. Le Pen, M. Shahrooei, E. Goudouris, A. Shirkani, M. Changi-Ashtiani, H. Rokni-Zadeh, E. H. Sayar, I. Reisli, A. Lefevre-Utile, D. Zijlmans, A. Jurado, R. Pholien, S. Drutman, S. Belkaya, A. Cobat, R. Boudewijns, D. Jochmans, J. Neyts, Y. Seeleuthner, L. Lorenzo-Diaz, C. Enemchukwu, I. Tietjen, H. H. Hoffmann, M. Momenilandi, L. Poyhonen, M. M. Siqueira, S. M. B. de Lima, D. C. de Souza Matos, A. Homma, M. L. S. Maia, T. A. da Costa Barros, P. M. N. de Oliveira, E. C. Mesquita, R. Gijsbers, S. Y. Zhang, S. J. Seligman, L. Abel, P. Hertzog, N. Marr, R. M. Martins, I. Meyts, Q. Zhang, M. R. MacDonald, C. M. Rice, J. L. Casanova, E. Jouanguy, and X. Bossuyt. 2019. Inherited IFNAR1 deficiency in otherwise healthy patients with adverse reaction to measles and yellow fever live vaccines. J Exp Med 216:2057-2070.
Hernandez, N., I. Melki, H. Jing, T. Habib, S. S. Y. Huang, J. Danielson, T. Kula, S. Drutman, S. Belkaya, V. Rattina, L. Lorenzo-Diaz, A. Boulai, Y. Rose, N. Kitabayashi, M. P. Rodero, C. Dumaine, S. Blanche, M. N. Lebras, M. C. Leung, L. S. Mathew, B. Boisson, S. Y. Zhang, S. Boisson-Dupuis, S. Giliani, D. Chaussabel, L. D. Notarangelo, S. J. Elledge, M. J. Ciancanelli, L. Abel, Q. Zhang, N. Marr, Y. J. Crow, H. C. Su, and J. L. Casanova. 2018. Life-threatening influenza pneumonitis in a child with inherited IRF9 deficiency. J Exp Med 215:2567-2585.
Howe, H. S., and B. P. L. Leung. 2019. Anti-Cytokine Autoantibodies in Systemic Lupus Erythematosus. Cells 9(1):72. doi: 10.3390/cells9010072.
Itan, Y., L. Shang, B. Boisson, M. J. Ciancanelli, J. G. Markle, R. Martinez-Barricarte, E. Scott, I. Shah, P. D. Stenson, J. Gleeson, D. N. Cooper, L. Quintana-Murci, S. Y. Zhang, L. Abel, and J. L. Casanova. 2016. The mutation significance cutoff: gene-level thresholds for variant predictions. Nat Methods 13:109-110.
Kircher, M., D. M. Witten, P. Jain, B. J. O'Roak, G. M. Cooper, and J. Shendure. 2014. A general framework for estimating the relative pathogenicity of human genetic variants. Nat Genet 46:310-315. Kreins, A. Y., M. J. Ciancanelli, S. Okada, X. F. Kong, N. Ramirez-Alejo, S. S. Kilic, J. El Baghdadi, S.
Nonoyama, S. A. Mahdaviani, F. Ailal, A. Bousfiha, D. Mansouri, E. Nievas, C. S. Ma, G. Rao, A. Bernasconi, H. Sun Kuehn, J. Niemela, J. Stoddard, P. Deveau, A. Cobat, S. El Azbaoui, A. Sabri, C. K. Lim, M. Sundin, D. T. Avery, R. Halwani, A. V. Grant, B. Boisson, D. Bogunovic, Y. Itan, M. Moncada-Velez, R. Martinez-Barricarte, M. Migaud, C. Deswarte, L. Alsina, D. Kotlarz, C. Klein, I. Muller-Fleckenstein, B. Fleckenstein, V. Cormier-Daire, S. Rose-John, C. Picard, L. Hammarstrom, A. Puel, S. Al-Muhsen, L. Abel, D. Chaussabel, S. D. Rosenzweig, Y. Minegishi, S. G. Tangye, J. Bustamante, J. L.
Casanova, and S. Boisson-Dupuis. 2015. Human TYK2 deficiency: Mycobacterial and viral infections without hyper-IgE syndrome. J Exp Med 212:1641-1662.
Ku, C. L., C. Y. Chi, H. von Bernuth, and R. Doffinger. 2020. Autoantibodies against cytokines: phenocopies of primary immunodeficiencies? Hum Genet 139:783-794.
Lazear, H. M., J. W. Schoggins, and M. S. Diamond. 2019. Shared and Distinct Functions of Type I and Type III Interferons. Immunity 50:907-923.
Lecomte, E., G. Laureys, F. Verbeke, C. Domingo Carrasco, M. Van Esbroeck, and R. Huits. 2020. A clinician's perspective on yellow fever vaccine-associated neurotropic disease. J Travel Med 27(7):taaa172. doi: 10.1093/jtm/taaa172.
Li, H., and R. Durbin. 2009. Fast and accurate short read alignment with Burrows-Wheeler transform. Bioinformatics 25:1754-1760.
Li, H., B. Handsaker, A. Wysoker, T. Fennell, J. Ruan, N. Homer, G. Marth, G. Abecasis, R. Durbin, and S. Genome Project Data Processing. 2009. The Sequence Alignment/Map format and SAMtools. Bioinformatics 25:2078-2079.
Lindsey, N. P., I. B. Rabe, E. R. Miller, M. Fischer, and J. E. Staples. 2016. Adverse event reports following yellow fever vaccination, 2007-13. J Travel Med 23:
Martin, M., T. F. Tsai, B. Cropp, G. J. Chang, D. A. Holmes, J. Tseng, W. Shieh, S. R. Zaki, I. Al-Sanouri, A. F. Cutrona, G. Ray, L. H. Weld, and M. S. Cetron. 2001. Fever and multisystem organ failure associated with 17D-204 yellow fever vaccination: a report of four cases. Lancet 358:98-104.
Mathian, A., S. Mouries-Martin, K. Dorgham, H. Devilliers, L. Barnabei, E. Ben Salah, F. Cohen-Aubart, L. Garrido Castillo, J. Haroche, M. Hie, M. Pineton de Chambrun, M. Miyara, D. Sterlin, M. Pha, D. Le Thi Huong, F. Rieux-Laucat, F. Rozenberg, G. Gorochov, and Z. Amoura. 2019. Monitoring Disease Activity in Systemic Lupus Erythematosus With Single-Molecule Array Digital Enzyme-Linked Immunosorbent Assay Quantification of Serum Interferon-alpha. Arthritis Rheumatol 71:756-765.
McKenna, A., M. Hanna, E. Banks, A. Sivachenko, K. Cibulskis, A. Kernytsky, K. Garimella, D. Altshuler, S. Gabriel, M. Daly, and M. A. DePristo. 2010. The Genome Analysis Toolkit: a MapReduce framework for analyzing next-generation DNA sequencing data. Genome Res 20:1297-1303.
Meager, A., K. Visvalingam, P. Peterson, K. Moll, A. Murumagi, K. Krohn, P. Eskelin, J. Perheentupa, E. Husebye, Y. Kadota, and N. Willcox. 2006. Anti-interferon autoantibodies in autoimmune polyendocrinopathy syndrome type 1. PLoS Med 3:e289.
Monath, T. P. 2005. Yellow fever vaccine. Expert Rev Vaccines 4:553-574. Monath, T. P., M. S. Cetron, K. McCarthy, R. Nichols, W. T. Archambault, L. Weld, and P. Bedford. 2005. Yellow fever 17D vaccine safety and immunogenicity in the elderly. Hum Vaccin 1:207-214.
Ng, P. C., and S. Henikoff. 2001. Predicting deleterious amino acid substitutions. Genome Res 11:863-874.
Notarangelo, L. D., R. Bacchetta, J. L. Casanova, and H. C. Su. 2020. Human inborn errors of immunity: An expanding universe. Sci Immunol 5(49):eabb1662. doi: 10.1126/sciimmunol.abb1662.
Panem, S., I. J. Check, D. Henriksen, and J. Vilcek. 1982. Antibodies to alpha-interferon in a patient with systemic lupus erythematosus. J Immunol 129:1-3.
Pulendran, B., J. Miller, T. D. Querec, R. Akondy, N. Moseley, O. Laur, J. Glidewell, N. Monson, T. Zhu, H. Zhu, S. Staprans, D. Lee, M. A. Brinton, A. A. Perelygin, C. Vellozzi, P. Brachman, Jr., S. Lalor, D. Teuwen, R. B. Eidex, M. Cetron, F. Priddy, C. del Rio, J. Altman, and R. Ahmed. 2008. Case of yellow fever vaccine-associated viscerotropic disease with prolonged viremia, robust adaptive immune responses, and polymorphisms in CCRS and RANTES genes. J Infect Dis 198:500-507.
Russell-Harde, D., T. C. Wagner, M. R. Rani, D. Vogel, O. Colamonici, R. M. Ransohoff, B. Majchrzak, E. Fish, H. D. Perez, and E. Croze. 2000. Role of the intracellular domain of the human type I interferon receptor 2 chain (IFNAR2c) in interferon signaling. Expression of IFNAR2c truncation mutants in U5A cells. J Biol Chem 275:23981-23985.
Sanchez-Felipe, L., T. Vercruysse, S. Sharma, J. Ma, V. Lemmens, D. Van Looveren, M. P. A. Javarappa, R. Boudewijns, B. Malengier-Devlies, L. Liesenborghs, S. J. F. Kaptein, C. De Keyzer, L. Bervoets, S. Debaveye, M. Rasulova, L. Seldeslachts, L. H. Li, S. Jansen, M. B. Yakass, B. E. Verstrepen, K. P. Boszormenyi, G. Kiemenyi-Kayere, N. van Driel, O. Quaye, X. Zhang, S. Ter Horst, N. Mishra, W. Deboutte, J. Matthijnssens, L. Coelmont, C. Vandermeulen, E. Heylen, V. Vergote, D. Schols, Z. Wang, W. Bogers, T. Kuiken, E. Verschoor, C. Cawthorne, K. Van Laere, G. Opdenakker, G. V. Velde, B. Weynand, D. E. Teuwen, P. Matthys, J. Neyts, H. Jan Thibaut, and K. Dallmeier. 2020. A single-dose live-attenuated YF17D-vectored SARS-CoV-2 vaccine candidate. Nature doi:10.1038/341586-020-035-9.
Seligman, S. J. 2014. Risk groups for yellow fever vaccine-associated viscerotropic disease (YEL-AVD). Vaccine 32:5769-5775.
Seligman, S. J., and J. L. Casanova. 2016. Yellow fever vaccine: worthy friend or stealthy foe? Expert Rev Vaccines 15:681-691.
Slesak, G., M. Gabriel, C. Domingo, and J. Schafer. 2017. [Severe Yellow fever vaccine associated disease: a case report and current overview]. Dtsch Med Wochenschr 142:1219-1222.
Tangye, S. G., W. Al-Herz, A. Bousfiha, T. Chatila, C. Cunningham-Rundles, A. Etzioni, J. L. Franco, S. M. Holland, C. Klein, T. Morio, H. D. Ochs, E. Oksenhendler, C. Picard, J. Puck, T. R. Torgerson, J. L. Casanova, and K. E. Sullivan. 2020. Human Inborn Errors of Immunity: 2019 Update on the Classification from the International Union of Immunological Societies Expert Committee. J Clin Immunol 40:24-64.
Vasconcelos, P. F., E. J. Luna, R. Galler, L. J. Silva, T. L. Coimbra, V. L. Barros, T. P. Monath, S. G. Rodigues, C. Laval, Z. G. Costa, M. F. Vilela, C. L. Santos, P. M. Papaiordanou, V. A. Alves, L. D. Andrade, H. K. Sato, E. S. Rosa, G. B. Froguas, E. Lacava, L. M. Almeida, A. C. Cruz, I. M. Rocco, R. T. Santos, O. F. Oliva, and G. Brazilian Yellow Fever Vaccine Evaluation. 2001. Serious adverse events associated with yellow fever 17DD vaccine in Brazil: a report of two cases. Lancet 358:91-97.
Walter, J. E., L. B. Rosen, K. Csomos, J. M. Rosenberg, D. Mathew, M. Keszei, B. Ujhazi, K. Chen, Y. N. Lee, I. Tirosh, K. Dobbs, W. Al-Herz, M. J. Cowan, J. Puck, J. J. Bleesing, M. S. Grimley, H. Malech, S. S. De Ravin, A. R. Gennery, R. S. Abraham, A. Y. Joshi, T. G. Boyce, M. J. Butte, K. C. Nadeau, I. Balboni, K. E. Sullivan, J. Akhter, M. Adeli, R. A. El-Feky, D. H. El-Ghoneimy, G. Dbaibo, R. Wakim, C. Azzari, P. Palma, C. Cancrini, K. Capuder, A. Condino-Neto, B. T. Costa-Carvalho, J. B. Oliveira, C. Roifman, D. Buchbinder, A. Kumanovics, J. L. Franco, T. Niehues, C. Schuetz, T. Kuijpers, C. Yee, J. Chou, M. J. Masaad, R. Geha, G. Uzel, R. Gelman, S. M. Holland, M. Recher, P. J. Utz, S. K. Browne, and L. D. Notarangelo. 2015. Broad-spectrum antibodies against self-antigens and cytokines in RAG deficiency. J Clin Invest 125:4135-4148.
Watford, W. T., and J. J. O'Shea. 2006. Human tyk2 kinase deficiency: another primary immunodeficiency syndrome. Immunity 25:695-697.
Wilmes, S., O. Beutel, Z. Li, V. Francois-Newton, C. P. Richter, D. Janning, C. Kroll, P. Hanhart, K. Hotte, C. You, G. Uze, S. Pellegrini, and J. Piehler. 2015. Receptor dimerization dynamics as a regulatory valve for plasticity of type I interferon signaling. J Cell Biol 209:579-593.
Yi, Z., L. Sperzel, C. Nurnberger, P. J. Bredenbeek, K. J. Lubick, S. M. Best, C. T. Stoyanov, L. M. Law, Z. Yuan, C. M. Rice, and M. R. MacDonald. 2011. Identification and characterization of the host protein DNAJC14 as a broadly active flavivirus replication modulator. PLoS Pathog 7:e1001255.
Zhang, Q., P. Bastard, A. Bolze, E. Jouanguy, S. Y. Zhang, A. Cobat, L. D. Notarangelo, H. C. Su, L. Abel, and J. L. Casanova. 2020a. Life-Threatening COVID-19: Defective Interferons Unleash Excessive Inflammation. MED 1:14-20.
Zhang, Q., P. Bastard, Z. Liu, J. Le Pen, M. Moncada-Velez, J. Chen, M. Ogishi, I. K. D. Sabli, S. Hodeib, C. Korol, J. Rosain, K. Bilguvar, J. Ye, A. Bolze, B. Bigio, R. Yang, A. A. Arias, Q. Zhou, Y. Zhang, F. Onodi, S. Korniotis, L. Karpf, Q. Philippot, M. Chbihi, L. Bonnet-Madin, K. Dorgham, N. Smith, W. M. Schneider, B. S. Razooky, H. H. Hoffmann, E. Michailidis, L. Moens, J. E. Han, L. Lorenzo, L. Bizien, P. Meade, A. L. Neehus, A. C. Ugurbil, A. Corneau, G. Kerner, P. Zhang, F. Rapaport, Y. Seeleuthner, J. Manry, C. Masson, Y. Schmitt, A. Schluter, T. Le Voyer, T. Khan, J. Li, J. Fellay, L. Roussel, M. Shahrooei, M. F. Alosaimi, D. Mansouri, H. Al-Saud, F. Al-Mulla, F. Almourfi, S. Z. Al-Muhsen, F. Alsohime, S. Al Turki, R. Hasanato, D. van de Beek, A. Biondi, L. R. Bettini, M. D′Angio, P. Bonfanti, L. Imberti, A. Sottini, S. Paghera, E. Quiros-Roldan, C. Rossi, A. J. Oler, M. F. Tompkins, C. Alba, I. Vandernoot, J. C. Goffard, G. Smits, I. Migeotte, F. Haerynck, P. Soler-Palacin, A. Martin-Nalda, R. Colobran, P. E. Morange, S. Keles, F. Colkesen, T. Ozcelik, K. K. Yasar, S. Senoglu, S. N. Karabela, C. Rodriguez-Gallego, G. Novelli, S. Hraiech, Y. Tandjaoui-Lambiotte, X. Duval, C. Laouenan, C.-S. Clinicians, C. Clinicians, C. G. Imagine, C. C. S. G. French, V. C. C. Co, U. M. C. C.-B. Amsterdam, C. H. G. Effort, N.-U. T. C. I. Group, A. L. Snow, C. L. Dalgard, J. D. Milner, D. C. Vinh, T. H. Mogensen, N. Marr, A. N. Spaan, B. Boisson, S. Boisson-Dupuis, J. Bustamante, A. Puel, M. J. Ciancanelli, I. Meyts, T. Maniatis, V. Soumelis, A. Amara, M. Nussenzweig, A. Garcia-Sastre, F. Krammer, A. Pujol, D. Duffy, R. P. Lifton, S. Y. Zhang, G. Gorochov, V. Beziat, E. Jouanguy, V. Sancho-Shimizu, C. M. Rice, L. Abel, L. D. Notarangelo, A. Cobat, H. C. Su, and J. L. Casanova. 2020b. Inborn errors of type I IFN immunity in patients with life-threatening COVID-19. Science 370(6515):eabd4570. doi: 10.1126/science.abd4570.

### Example 4

### Autoantibodies Neutralizing Type I IFNs are Common in the Elderly and Account for at Least 20% of COVID-19 Deaths

Circulating autoantibodies (auto-Abs) neutralizing very high concentrations (10 ng/mL, in plasma 1/10) of IFN-α and/or -ω have been found in about 10% of patients with critical COVID-19 pneumonia, but not in subjects with asymptomatic infections. We detected auto-Abs neutralizing 100-fold lower, more physiological, concentrations of IFN-α and/or -ω (100 pg/mL, in plasma 1/10) in at least 15% of 2,248 patients with critical COVID-19 pneumonia, including 22% of 290 patients over the age of 80 years. These antibodies were also detected in 21% of 865 individuals aged 20-99 (mean: 70 years) who died of COVID-19 pneumonia. We also show, in a sample of 33,352 uninfected subjects, that the prevalence of auto-Abs neutralizing high concentrations of type I IFNs sharply increases with age, with such antibodies present in 0.16% of individuals between 18 and 69 years, 1% of individuals between 70 and 79 years, and 2.9% of individuals after 80 years of age. Moreover, in a subsample of 816 individuals, the proportion of subjects carrying auto-Abs neutralizing 100-fold lower concentrations was even greater, at 0.53% of individuals between 60 and 70 years, 1.3% between 70 and 80 years, and 3.7% over 80 years of age. Auto-Abs neutralizing type I IFNs predate SARS-CoV-2 infection, sharply increase in prevalence after the age of 70 years, and account for more than 20% of cases of both critical COVID-19 in subjects over the age of 80 years and fatal COVID-19 at all ages.

Introduction

Since the start of the COVID-19 pandemic, in December 2019, more than 150 million people have been infected with SARS-CoV-2, resulting in at least three million deaths worldwide, and more likely six to eight million. Inter-individual clinical variability in the course of acute infection is vast, extending from silent or mild infection in about 90% of subjects to pneumonia and respiratory failure, in less than 10% and 2% of cases, respectively, who require hospitalization. Age is the major epidemiological risk factor for hospitalization or death from pneumonia, the risk doubling with every five years of age (1, 2). The frequencies of critical disease and death from COVID-19 are higher in men than in women (3). With the COVID Human Genetic Effort (4), we previously reported that inborn errors of TLR3- and IRF7-dependent type I IFN induction and amplification can underlie life-threatening COVID-19 pneumonia in a small subset of patients (5). Autosomal dominant disorders were found in 19 patients, but our cohort also included four previously healthy unrelated adults aged 25 to 50 years with autosomal recessive, complete IRF7 (N=2) or IFNAR1 (N=2) deficiency. These findings indicate that type I IFN immunity is essential for protective immunity to respiratory infection with SARS-CoV-2 but can be surprisingly redundant otherwise. We also reported that an autoimmune phenocopy of inborn errors of type I IFN immunity can underlie critical COVID-19 pneumonia (6). Indeed, auto-Abs neutralizing 10 ng/mL IFN-α2 and/or -ω were found in the blood of at least 10% of an international cohort of patients with life-threatening COVID-19 pneumonia, but in none of the individuals with asymptomatic or paucisymptomatic infection tested (6). These auto-Abs were detected in serum or plasma diluted 1/10. The auto-Abs in the patients' undiluted blood are probably, therefore, able to neutralize as much as 100 ng/mL IFN-α2 and/or -ω. These auto-Abs were mostly found in men (95%) and in the elderly (half the patients with antibodies being over the age of 65 years) (6). These findings were later replicated in independent cohorts from Amsterdam, Lyon, Madrid, New Haven, and San Francisco (7-12).

These IgG auto-Abs against type I IFNs were found in about 0.3% of a general population sample of 1,227 subjects collected before the pandemic and aged 20 to 65 years, suggesting that they predated SARS-CoV-2 infection and caused critical COVID-19 rather than being triggered by it (6). Moreover, the production of these antibodies can be genetically driven, and can begin during early childhood, as attested by their presence in almost all patients with autoimmune polyendocrine syndrome type-1 (APS-1) due to germline mutations of AIRE (13-15). These patients are, therefore, at very high risk of developing severe or critical COVID-19 pneumonia (16). These auto-Abs are also found in patients with combined immunodeficiency and hypomorphic mutations of RAG1 or RAG2 (17), in men with immunodysregulation polyendocrinopathy enteropathy X-linked (IPEX) and mutations of FOXP3 (18), and in women with incontinentia pigmenti and heterozygous null mutations of X-linked NEMO (6). They are also seen in patients treated with IFN-α or IFN-β (19, 20), in patients with systemic lupus erythematosus (21), thymoma (22), or with myasthenia gravis (23). Finally, they underlie a third of adverse reactions to the live attenuated YFV-17D vaccine, further suggesting that they were present in patients with critical COVID-19 before SARS-CoV-2 infection (24). Remarkably, the auto-Abs neutralized the protective effect of ˜384 pg/mL IFN-α2 against SARS-CoV-2 or YFV-17D in vitro, even when diluted by >1/1,000, for all patients tested (6). As blood IFN-α concentrations during acute asymptomatic or paucisymptomatic SARS-CoV-2 infection typically range from 1 to 100 pg/mL (25, 26), and IFN-α levels in the respiratory tract may be even lower, we hypothesized that auto-Abs neutralizing concentrations of type I IFNs below 10 ng/mL (from plasma diluted 1/10) may underlie life-threatening COVID-19 pneumonia in more than 10% of cases. We also hypothesized that the prevalence of auto-Abs against type I IFNs in the general, uninfected, population may increase with age and that these antibodies may be more common in men than in women.

High and Intermediate Levels of IgG Auto-Abs Against Type I IFNs in ˜20% of Critical COVID-19 Patients

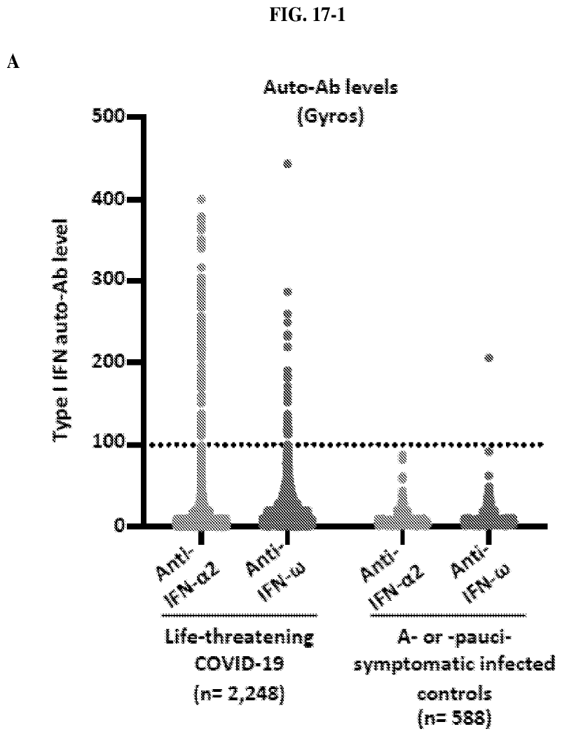

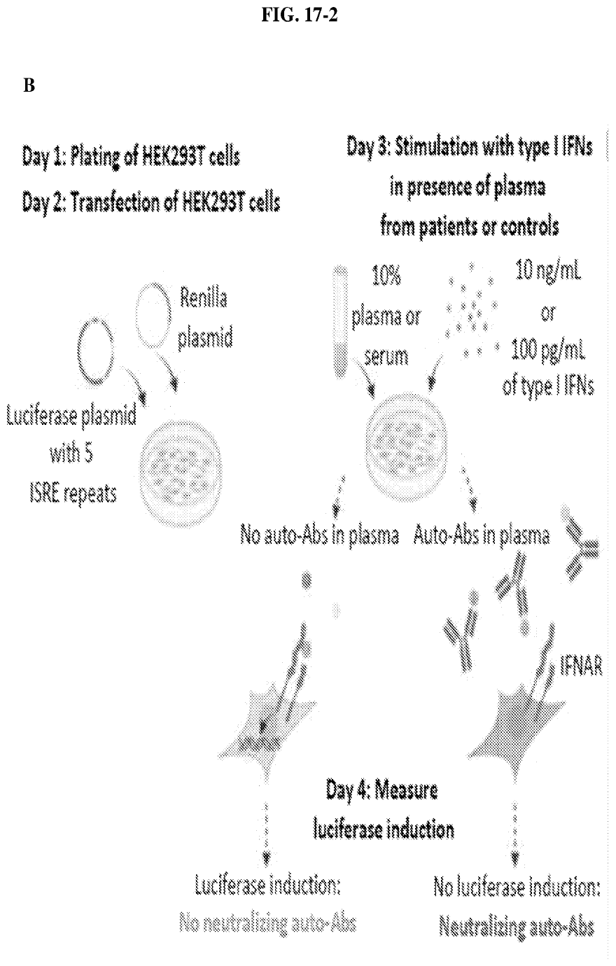

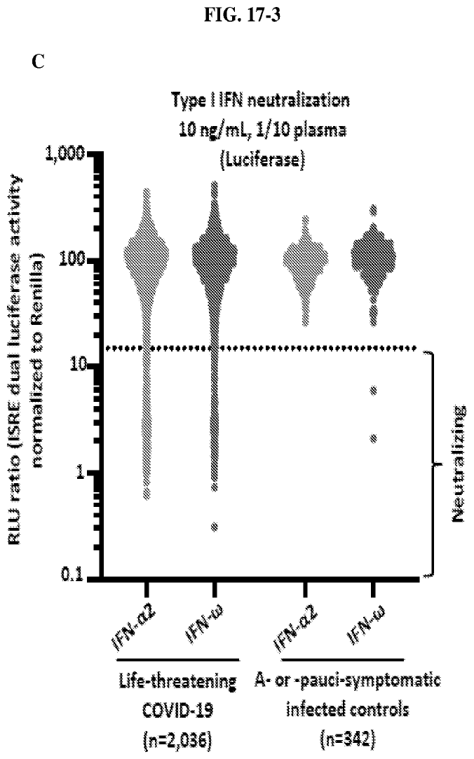

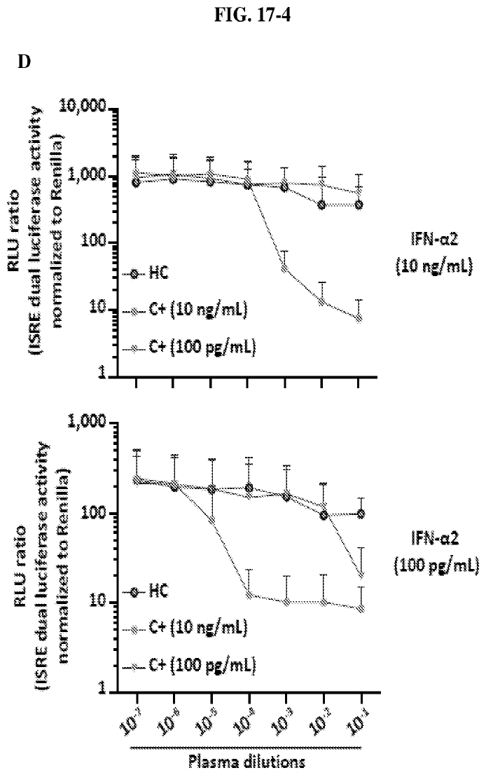

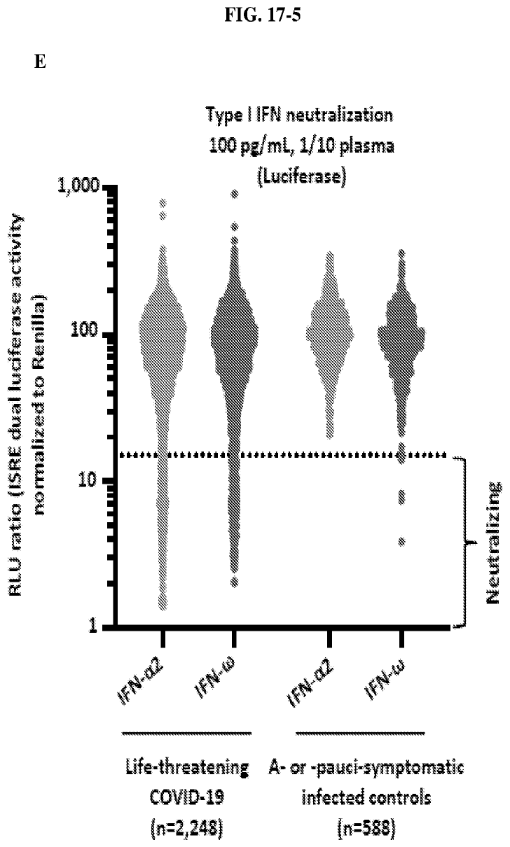

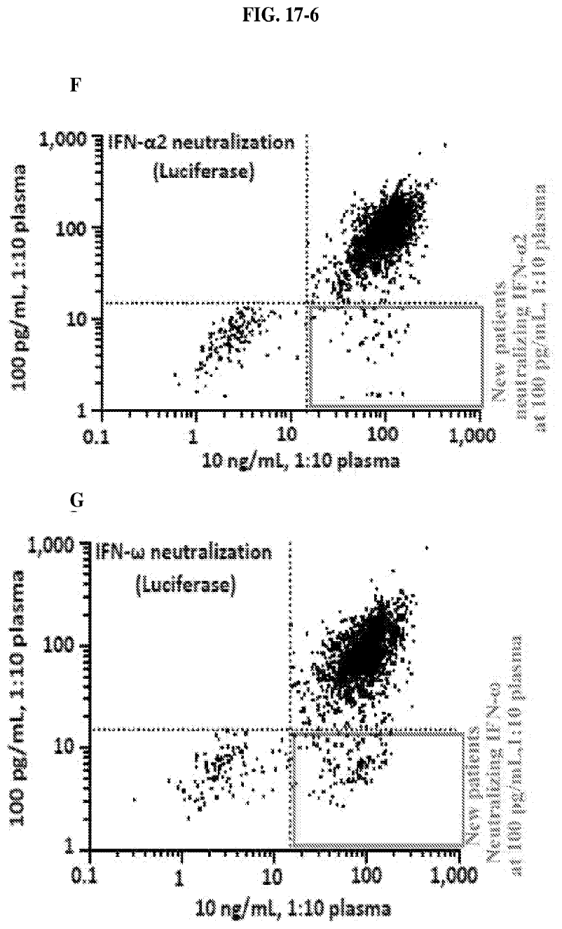

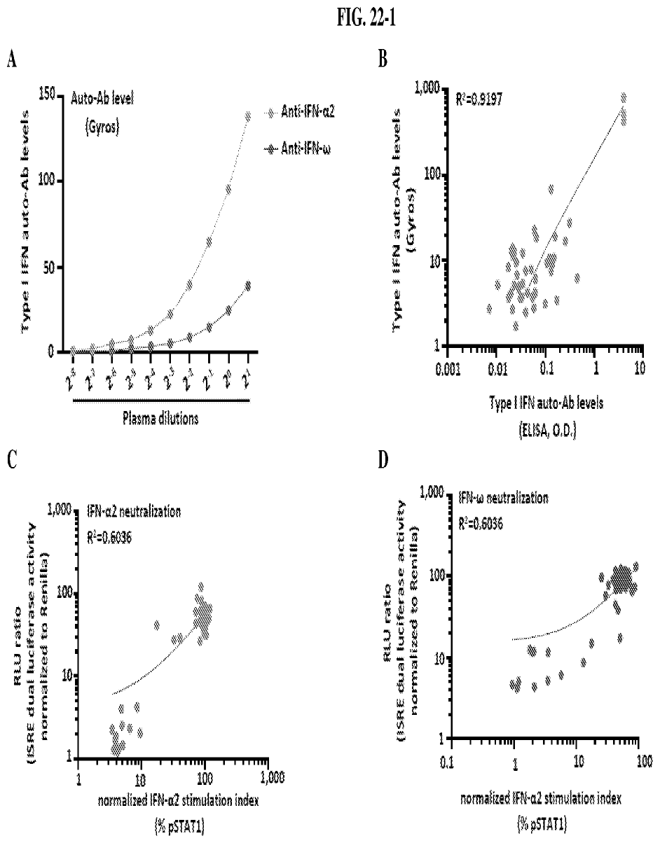

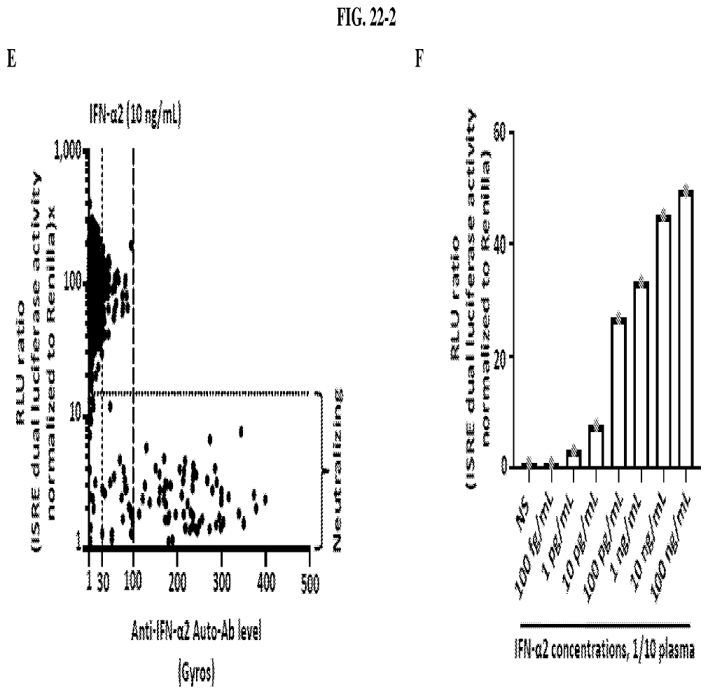

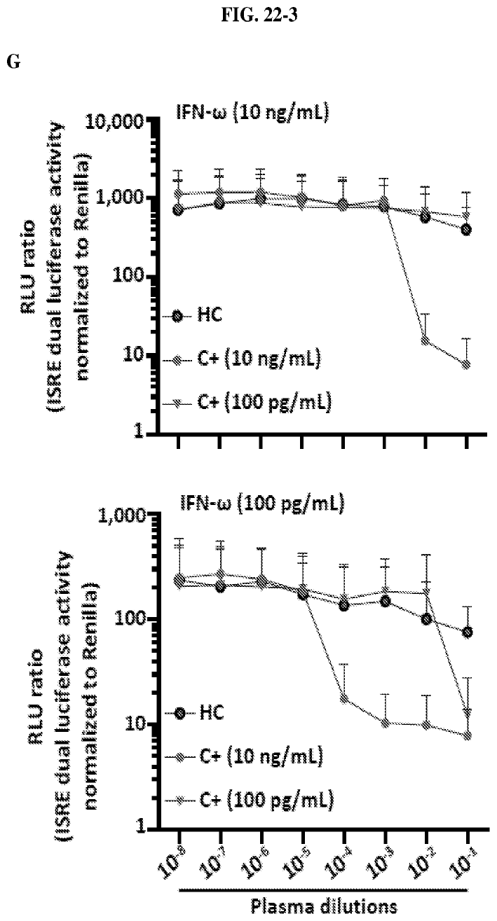

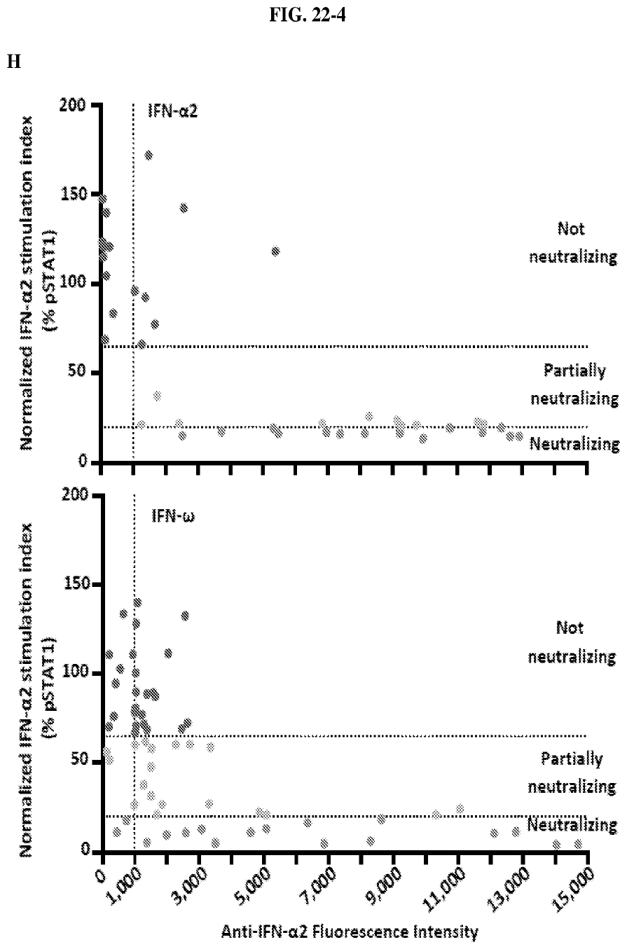

We searched for auto-Abs against IFN-α2 and -ω in a cohort of patients hospitalized with life-threatening COVID-19 pneumonia (hereafter referred to as “patients”), including 566 of the initial 987 patients with critical COVID-19 pneumonia for whom residual samples were available (6), and 588 individuals with asymptomatic or paucisymptomatic SARS-CoV-2 infection (the “controls”), including 136 samples from the initial control cohort of 663 individuals (6). We did not test patients with moderate or severe pneumonia (5, 6). We first set up novel, sensitive, and robust assays for the detection of circulating IgG auto-Abs. We used Gyros technology (27), a high-throughput automated ELISA (enzyme-linked immunosorbent assay) yielding reproducible results (FIG. 22A), and screened all patients and controls from our COVID-19 cohort (FIG. 17A). We confirmed that the Gyros technique was as sensitive as the techniques previously used (ELISA and Luminex), and that all patients with high levels of anti-IFN-α2 and/or anti-IFN-ω auto-Abs on ELISA, as reported in our previous studies (defined as an optical density >0.5) had high levels of auto-Abs when assessed with Gyros (defined as levels >100) (FIG. 22B). We found high levels of anti-IFN-α2 and/or anti-IFN-ω auto-Abs in 9% of the patients in our cohort of critical patients (203 of 2,248), whereas none of the 588 asymptomatic or paucisymptomatic controls had high titers of auto-Abs against IFN-α2 and only one had high titers of auto-Abs against IFN-ω (Fisher's exact test, P<10⁻¹⁶) (FIG. 17A). We also found that another 11.5% of patients with critical COVID-19 (259 of 2,248) had intermediate levels of anti-IFN-α2 and/or IFN-ω auto-Abs in Gyros assays (defined as levels >30 and <100, based on the distribution observed in healthy controls), whereas this was the case for only 3% (17 of 588) of the individuals in our control cohort (Fisher exact test, P=9·10⁻¹¹). Importantly, none of the patients with high or intermediate auto-Abs carried inborn errors of TLR3- or TLR7-dependent type I IFN (5) (Asano et al, accompanying report submitted). Collectively, these findings replicate and extend our previous results and those of other groups (6-12), while also suggesting that intermediate levels of auto-Abs against type I IFNs may be neutralizing and underlie critical disease.

Auto-Abs Neutralizing 10 ng/mL IFN-α2 and/or -ω in 10% of the Patients

We investigated the ability of these auto-Abs to neutralize high concentrations of type I IFNs, as defined in our previous reports (10 ng/mL in medium containing 10% plasma or serum, the equivalent of 100 ng/mL in undiluted plasma). We tested not only the patients with high levels of auto-Abs, as in our previous study (6), but all the available patients (N=2,036) and controls (N=342) from our expanded cohort. We designed a high-throughput luciferase assay in which we transfected human embryonic kidney (HEK)-293T cells with a plasmid containing five interferon-sensitive response element (ISRE) repeats and a luciferase reporter. We stimulated these cells with an individual recombinant type I IFN (IFN-α2 or IFN-ω), in the presence of 10% plasma from patients or controls. We then measured firefly luciferase induction, normalized against Renilla luciferase activity (FIG. 17B). We confirmed the robustness of this assay by comparing its results with our previous pSTAT1 flow cytometry data (6). Consistent results were obtained for all 50 patients tested with both techniques (FIG. 22C, D). We then tested all patients and controls. We found that 10% (204 of 2,036) of the patients tested had auto-Abs that neutralized 10 ng/mL IFN-α2 and/or IFN-ω in 10% plasma, whereas none of the 342 controls had auto-Abs neutralizing IFN-α2 and only two had auto-Abs neutralizing IFN-ω (Fisher's exact test, P<10⁻¹²) (FIG. 17C) (Table 10). Plasma samples with high auto-Ab levels (>100) against IFN-α2 according to the Gyros assay were all neutralizing (FIG. 22E). In the patients with neutralizing auto-Abs, they were able to neutralize both IFN-α2 and IFN-ω in 112 of the 204 patients (55%), IFN-α2 alone in 75 patients (37%), and IFN-ω alone in 17 patients (8%), after stimulation with 10 ng/mL IFN-α2 or IFN-ω.

Auto-Abs Neutralizing 100 pg/mL IFN-α2 and/or -ω in >15% of the Patients

As the amounts of circulating type I IFNs in infected individuals are 100 to 1,000 times lower than the amounts tested previously (25, 26), we investigated the neutralization of more physiological concentrations of type I IFNs, by performing assays with 100 pg/mL type I IFN. We observed a robust response in our luciferase system, in the presence of 10% control plasma (FIG. 22F). The plasma or serum was diluted 1/10, so the concentration neutralized corresponds to 1 ng/mL IFN in circulating whole blood. With diluted plasma samples from a positive control, we gained at least 2 orders of magnitude of sensitivity in terms of neutralizing activity, providing proof-of-concept that these auto-Abs can neutralize lower, more physiological, amounts of type I IFNs (FIG. 17D, 22G), 100 times lower than the concentrations previously tested (6). We then retested all the available samples from our extended cohort. Overall, 15.7% of all patients tested (N=353 of 2,248) had circulating auto-Abs that neutralized 100 pg/mL IFN-α2 and/or IFN-ω in 10% plasma (FIG. 17E) (Table 10). These patients had a mean age of 63.5 years, and 73% were men. The auto-Abs of these patients neutralized both cytokines in 171 of cases (49%), only IFN-ω in 104 (29%) and only IFN-α2 in 78 (22%). All samples (N=177) tested that had previously been found to neutralize 10 ng/mL IFN-α2 or IFN-ω also neutralized 100 pg/mL IFN, as expected (FIG. 17F, G). Further dilution of a plasma sample from one patient neutralizing 100 pg/mL of type I IFNs led to a loss of neutralizing activity (FIG. 17D, 22G). None of the plasma samples from the 588 asymptomatic controls neutralized IFN-α2 and only five (0.85%) neutralized IFN-ω in these conditions (Fisher's exact test, P<10⁻¹⁶) (FIG. 17E) (Table 10). These five controls with auto-Abs were all under 60 years old, one was a woman and four were men. Among the 129 samples tested that were neutralizing at 100 pg/mL but not at 10 ng/mL, 31 neutralized IFN-α2 at 100 pg/mL, 84 neutralized IFN-ω at 100 pg/mL, and 14 neutralized both (FIG. 17F, G), further suggesting that auto-Abs neutralizing 100 pg/mL are causal for critical disease. None of the patients with these auto-Abs had inborn errors of TLR3- or TLR7-dependent type I IFN immunity (5) (Asano et al, accompanying report submitted). The serum/plasma samples being diluted 1/10 in these assays, these findings therefore suggest that more than 15% of patients with life-threatening COVID-19 have circulating auto-Abs neutralizing 1 ng/mL IFN-α2 and/or IFN-ω in vivo, a significantly greater proportion than the 10% of patients with auto-Abs neutralizing 100 ng/mL reported in previous studies (6-12).

The Auto-Abs Neutralize Low Concentrations of IFN-α2 Protective Against SARS-CoV-2

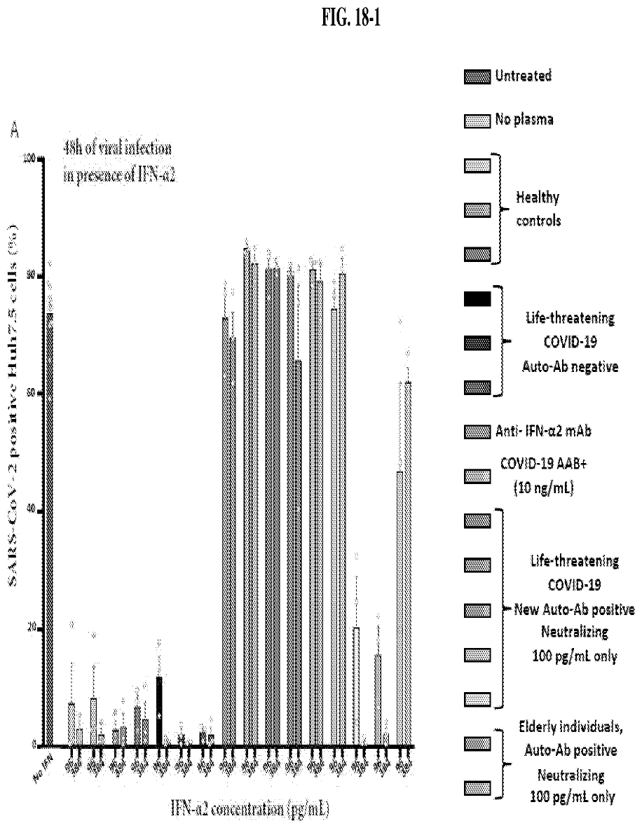

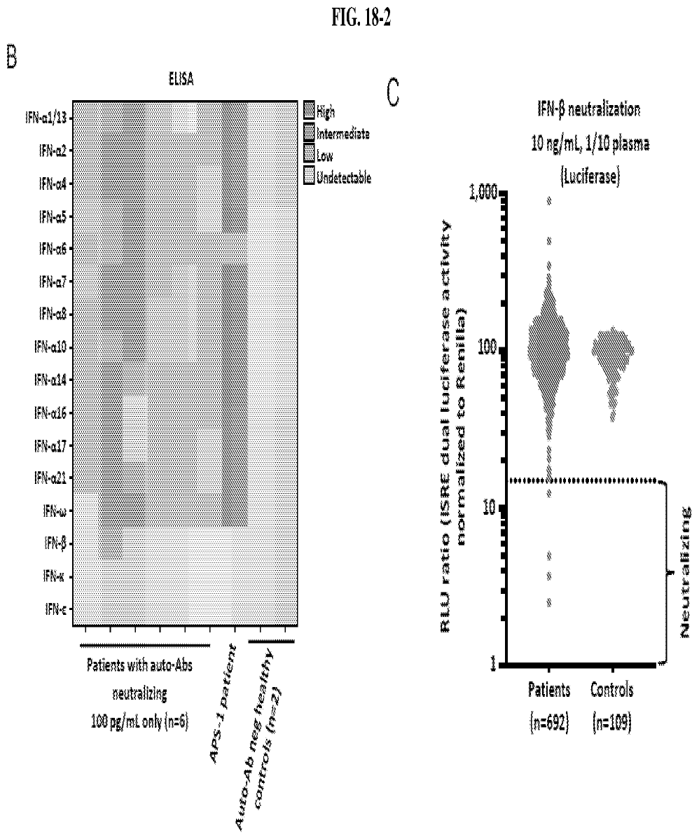

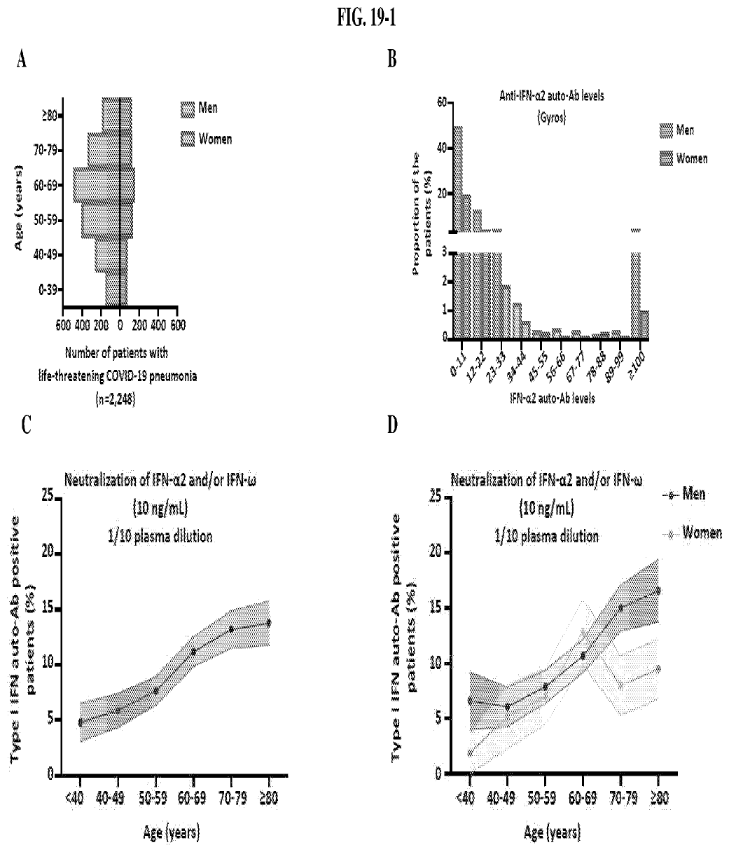

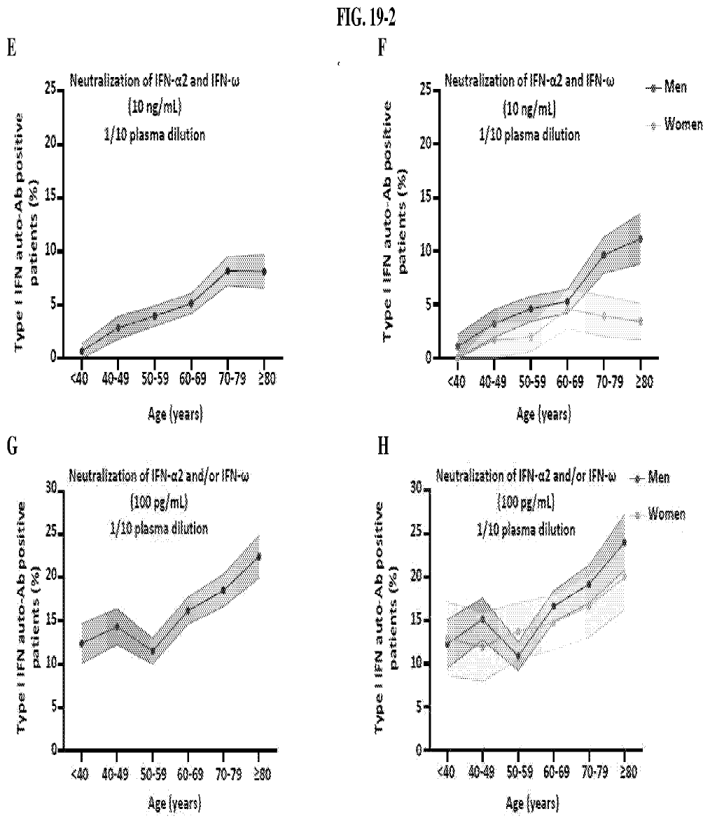

We previously reported that plasma diluted 1/100 from patients with auto-Abs against type I IFNs neutralized the ability of IFN-α2 (at a concentration of 20 pM, approximately 384 pg/mL) to block SARS-CoV-2 and YFV-17D replication in Huh-7.5 cells (see Example 2; 6, 24). Strikingly, this neutralization was seen in all patients tested, even for a 1,000-fold dilution, and, in most patients, it was more potent than the neutralizing effect of a commercially available neutralizing monoclonal Ab (mAb) against IFN-α2. These auto-Abs against type I IFNs were, therefore, able to neutralize IFN-α2 at concentrations well beyond physiological levels. We therefore hypothesized that patients with lower titers of auto-Abs against type I IFNs, which can neutralize 100 pg/mL but not 10 ng/mL in plasma diluted 1/10, would also neutralize the protective effect of IFN-α2 against SARS-CoV-2. We therefore performed our SARS-CoV-2 assay with 5 pM (˜96 pg/mL) or 20 pM (˜-384 pg/mL) IFN-α2, on five samples from patients with life-threatening COVID-19 and two samples from uninfected elderly individuals with auto-Abs neutralizing 100 pg/mL but not 10 ng/mL IFN-α2. As controls, we tested a commercial mAb against IFN-α2, a sample from a patient with auto-Abs neutralizing 10 ng/mL IFN-α2, and samples from three patients with life-threatening COVID-19 and three healthy controls without detectable auto-Abs against type I IFNs. We found that the 1/100 dilutions of plasma from five of the seven patients or elderly individuals with auto-Abs neutralizing 100 pg/mL IFN were able to neutralize the protective effect of ˜384 pg/mL IFN-α2 against SARS-CoV-2, while samples from the seven patients fully or partially neutralized ˜96 pg/m IFN-α2 (FIG. 18A). No such neutralizing effect was observed for any of the auto-Ab-negative controls. Overall, our findings indicate that auto-Abs against type I IFNs capable of neutralizing 100 pg/mL IFN in 1% plasma can block the protective effect of ˜96 pg/mL or ˜384 pg/mL IFN-α2 against SARS-CoV-2. These findings raise the possibility that even 100-fold lower levels of auto-Abs against type I IFNs, capable of neutralizing lower, physiological concentrations of 10 pg/mL IFN-α2, may be present in an even larger proportion of patients. The testing of this hypothesis will require the development of new, more sensitive methods for screening for neutralization.

Neutralization of Type I IFNs in the Absence of Detectable Auto-Abs Against IFN-α2 or -ω

The neutralization assays performed on all patients and controls revealed that some patients (15/186, 8%) with neutralizing activity against 10 ng/mL IFN-α2 and/or IFN-ω, as shown in luciferase assays, did not have high, or even intermediate levels of IgG auto-Abs in Gyros assays (FIG. 22E). Similar results were observed when using the Luminex assay for the titer and pSTAT1 induction (FIG. 22H). For these individuals, we assessed the prevalence of IgA and IgM auto-Abs against type I IFNs; we found that none of the patients tested (N=12) had detectable titers of IgA or IgM auto-Abs. We then tested the alternative hypothesis that these auto-Abs were directed against the IFNAR1 or IFNAR2 chain of type I IFN receptors, assessing the ability of plasma samples from these patients to neutralize IFN-β. None of the samples from these patients neutralized IFN-β, suggesting that the auto-Abs in these patients were not directed against IFNAR1 or IFNAR2. An alternative, plausible hypothesis is that the epitope recognized by the auto-Ab might be concealed by the binding of the cytokine to the plate (ELISA), biotin (Gyros), or the bead (Luminex) in the assays used. The auto-Abs in these patients might also potentially recognize an epitope concealed by the coupling of the cytokines to biotin (for the Gyros and Luminex assays). This hypothesis was supported by an in silico analysis, as the Luminex beads bind to lysine residue, and could thus block the access to a common epitope (15). This observation has important clinical implications, suggesting that a lack of detection of auto-Abs against type I IFNs does not rule out the possibility of such antibodies being present and having neutralization capacity.

The Auto-Abs Typically Neutralize the 13 IFN-α Forms and/or IFN-ω, and Rarely IFN-β

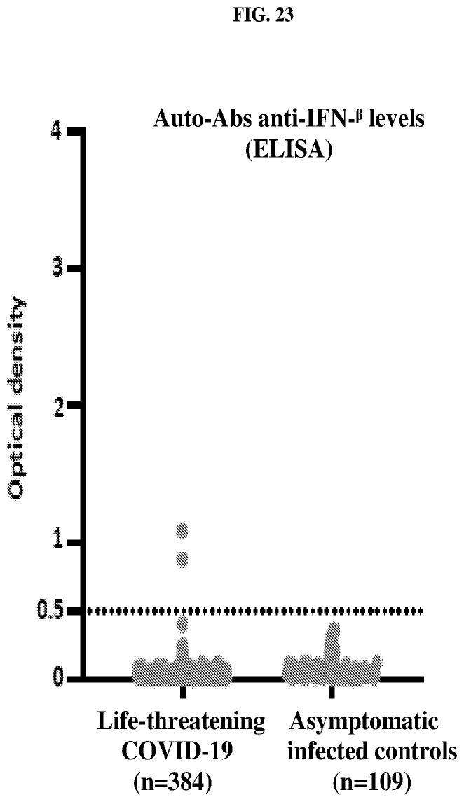

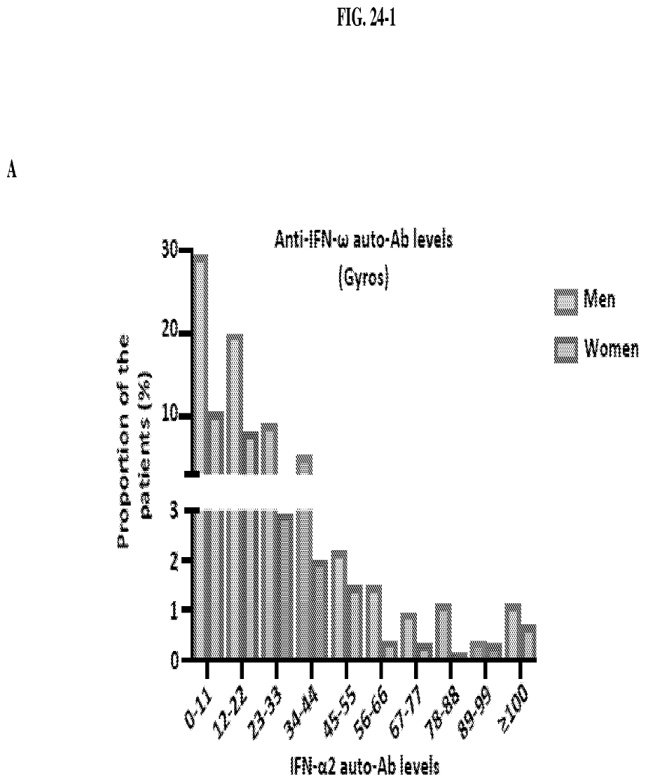

In six patients with auto-Abs neutralizing 100 pg/mL but not 10 ng/mL IFN-α2 and/or IFN-ω, we tested the reactivity of the antibodies against the 17 type I IFNs (the 13 IFN-α forms, IFN-ω, IFN-β, IFN-ε, and IFN-κ). Like patients with auto-Abs neutralizing 10 ng/mL type I IFNs (6), those capable of neutralizing only 100 pg/mL had detectable auto-Abs against most of the 13 IFN-α forms and/or IFN-ω, albeit at lower levels (FIG. 18B). Of the six patients with auto-Abs against IFN-α and/or IFN-ω tested, only one also had auto-Abs against IFN-β and none had detectable auto-Abs against IFN-ε or IFN-κ. Given the potential therapeutic use of IFN-β (28, 29), we also tested a larger number of patients and controls, including patients without auto-Abs against IFN-α or IFN-ω, for auto-Abs against this cytokine, assessing levels and neutralizing activity of auto-Abs against 10 ng/mL IFN-β. Of 50 patients with auto-Abs neutralizing 10 ng/mL and/or 100 pg/mL IFN-α2 and/or IFN-ω tested, only one (2%) had auto-Abs capable of neutralizing 10 ng/mL IFN-β (FIG. 18C). We then screened 642 patients without auto-Abs to IFN-α2 or IFN-ω neutralizing 100 pg/mL for neutralization activity against IFN-3. We found that only three patients (0.7%) had auto-Abs capable of neutralizing 10 ng/mL IFN-β, with no neutralizing activity in any of the asympto- or pauci-symptomatic infected controls tested (N=109) (FIG. 18C). By ELISA, two of the 642 patients tested had high levels of auto-Abs, while none of the controls tested had (N=109) (FIG. 23A). The third patient with auto-Abs neutralizing IFN-β had no detectable auto-Abs by ELISA. As these three patients with neutralizing auto-Abs against IFN-β did not have neutralizing auto-Abs against IFN-α2 or IFN-ω, this suggests that auto-Abs against IFN-β alone may also underlie life-threatening COVID-19 in rare cases. Overall, the patients with auto-Abs against IFN-α2 and/or IFN-ω capable of neutralizing 100 pg/mL IFN displayed patterns of reactivity to the 17 type I IFNs similar to those reported in previously described patients with auto-Abs neutralizing 10 ng/mL (6), as only a few (2%) had auto-Abs neutralizing only IFN-β. Finally, a few rare patients (0.7%) had auto-Abs that appeared to neutralize IFN-β only, and no such antibodies were detected in any of the controls tested.

Neutralizing Auto-Abs Against Type I IFNs in at Least 20% of Patients Over 80 Years of Age

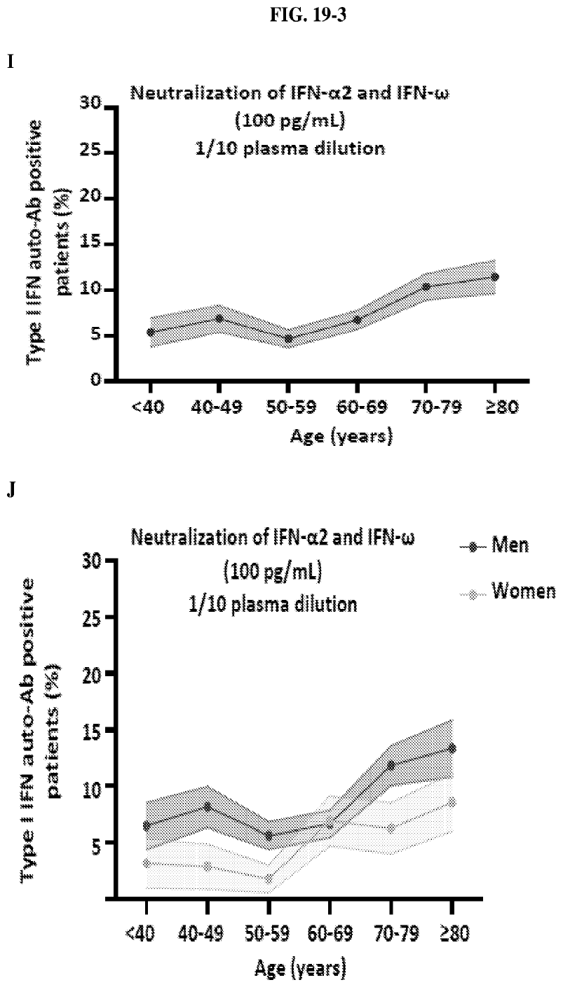

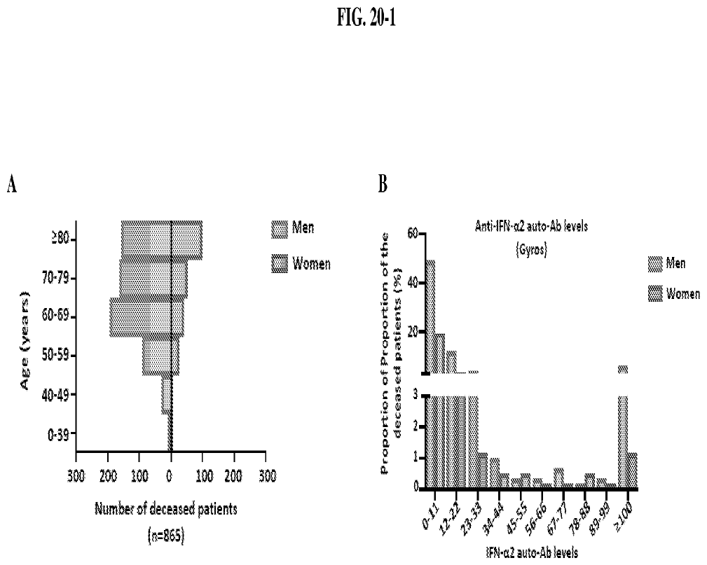

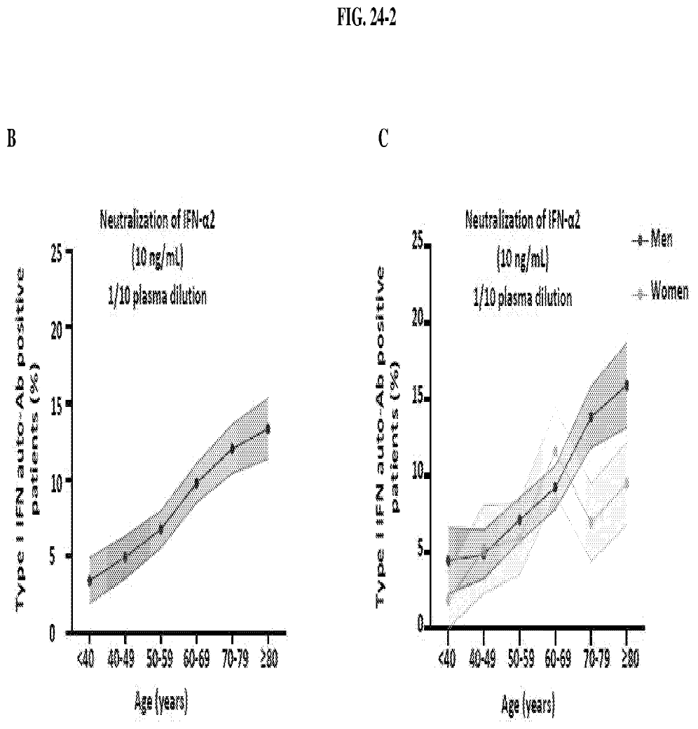

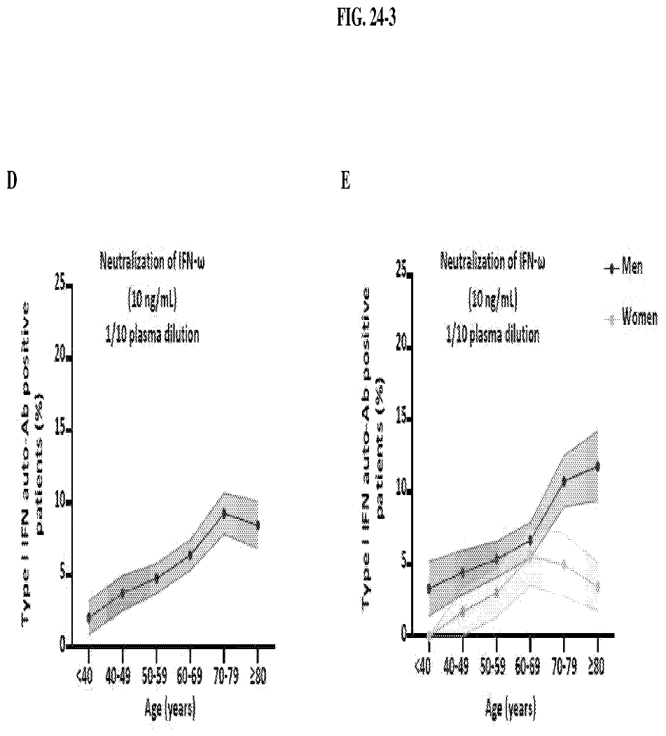

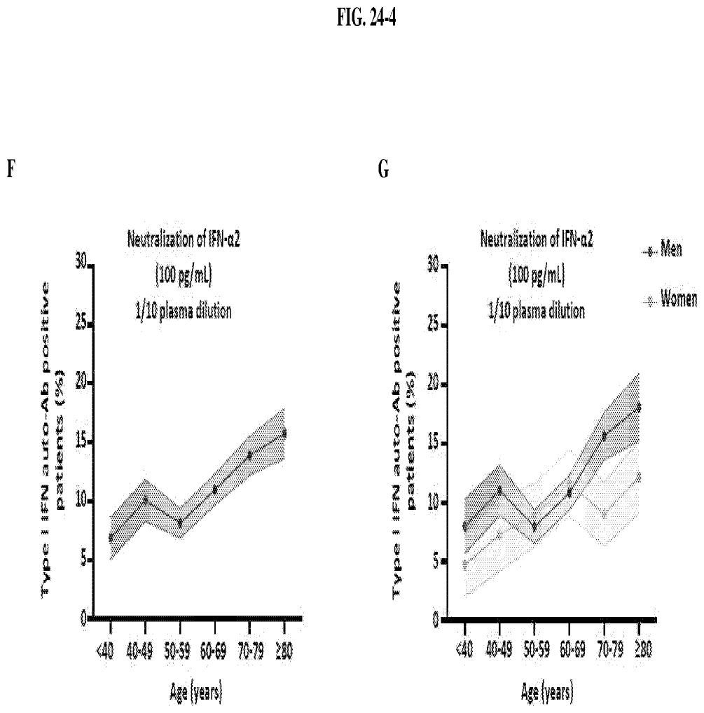

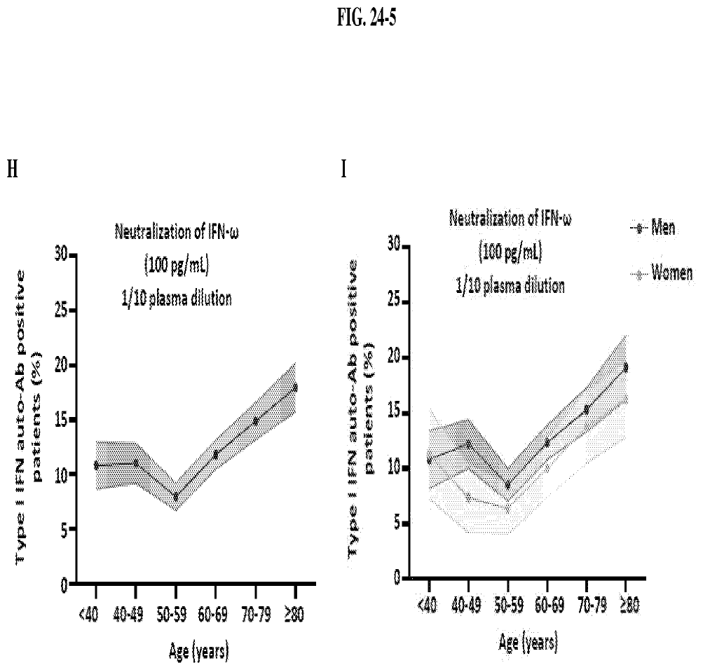

We investigated the risk factors for critical COVID-19, by stratifying the percentage of patients with auto-Abs against type I IFNs by age and sex. In our previous report, half the patients with auto-Abs and life-threatening COVID-19 were more than 65 years old and 95% were men (6). These results were confirmed by other groups, although the proportion of men was smaller (7-11). In our expanded cohort of patients with critical COVID-19 pneumonia (N=2,248), the mean age was 61 years and 73% were men (FIG. 19A). We assessed the percentage of individuals positive for neutralizing auto-Abs per decade of life and by sex, in patients with life-threatening COVID-19 (FIG. 19B-I, FIG. 24A-J) (Table 10). The mean age of patients with auto-Abs was 64 years old, and 74% were males. The proportion of auto-Abs significantly increased with age in both men and women (Wald test, P=7.10⁵), and the proportion of patients with auto-Abs was higher in men. In patients with auto-Abs neutralizing 10 ng/mL IFN, the increase was continuous, with auto-Abs against IFN-α2 and/or IFN-ω detected in 4.8% of patients under the age of 39 years, 5.9% of those between 40 and 49 years of age, 7.6% in those between 50 and 59 years of age, 11.2% in those between 60 and 69 years of age, and more than 13% in those over the age of 70 years (FIG. 19C-F, FIG. 24B-E). When compared with the critical patients without auto-Abs, the effect of sex on the proportion of patients with auto-Abs was not significant (Fisher exact test, P=0.13), while it was highly significant when compared with the controls (Fisher exact test, P<10⁻¹⁶). Similar results were obtained for patients with auto-Abs neutralizing 100 pg/mL IFN, but with even higher proportions (FIG. 19G-J, S3F-I) (Table 10). Indeed, the proportion of patients with auto-Abs ranged from 12% of patients below the age of 40 years, to more than 21% of those over 80 years of age again having a significant increase with age (Wald test, P=0.0004) (FIG. 19G-J, FIG. 24F-I). The increase in age was even more marked in men, with 24% of men over 80 years of age positive for these antibodies, versus only 20% of women in this age group. Overall, the prevalence of auto-Abs neutralizing 10 ng/ml and/or 100 pg/mL type I IFN increased sharply with age in all patients, particularly in men. A striking enrichment in patients with neutralizing auto-Abs against type I IFNs was observed in the elderly, with more than 20% of patients, and 24% of men, over the age of 80 years with critical COVID-19 having neutralizing auto-Abs against type I IFNs.

Neutralizing Auto-Abs Against Type I IFNs in at Least 20% of Deceased Patients

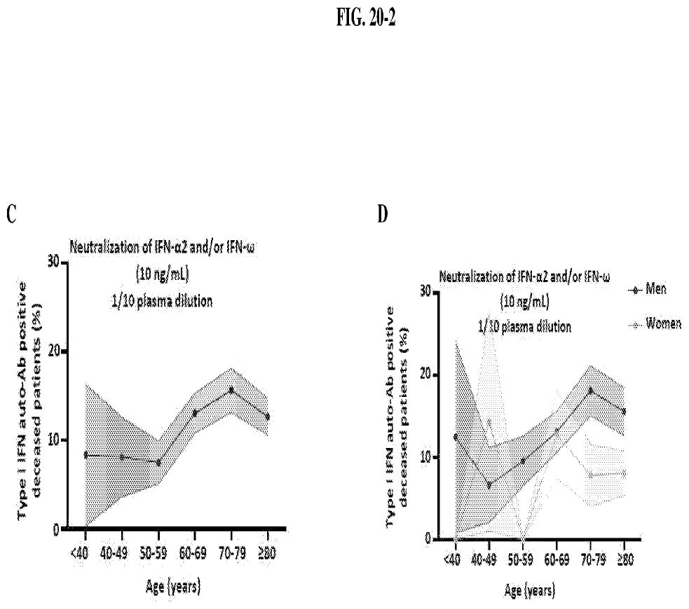

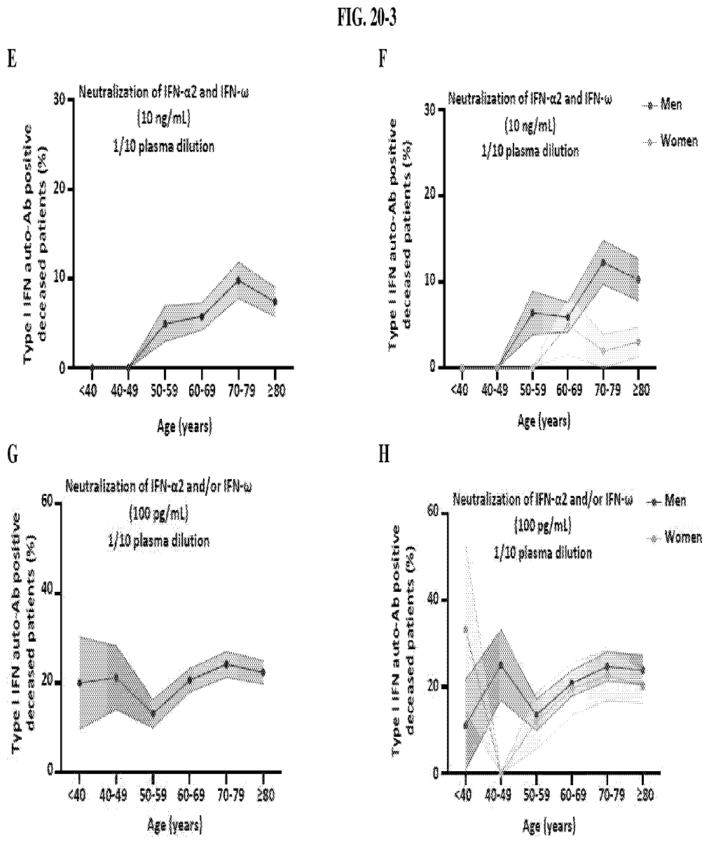

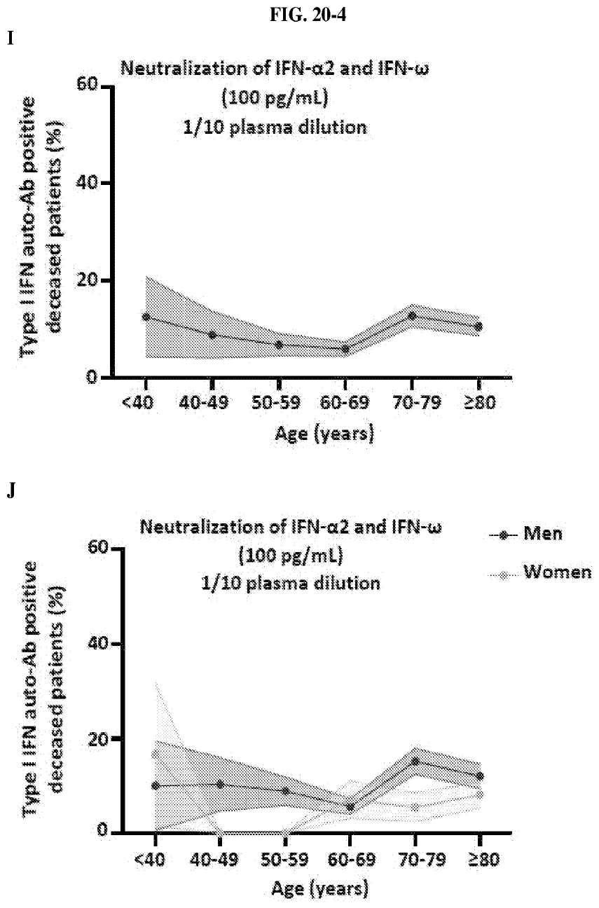

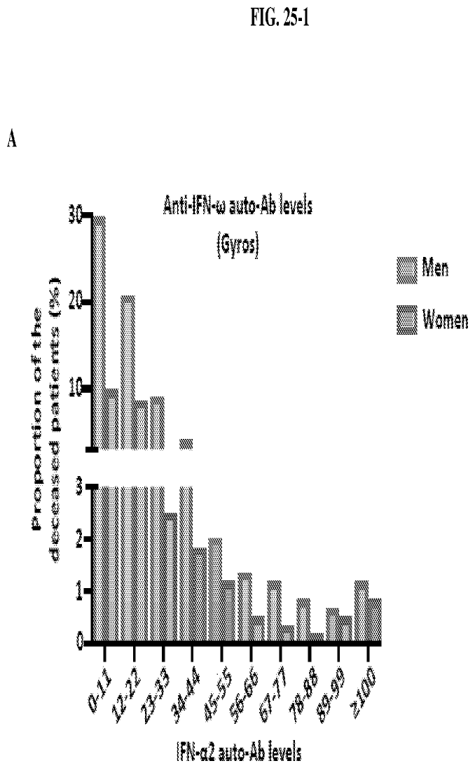

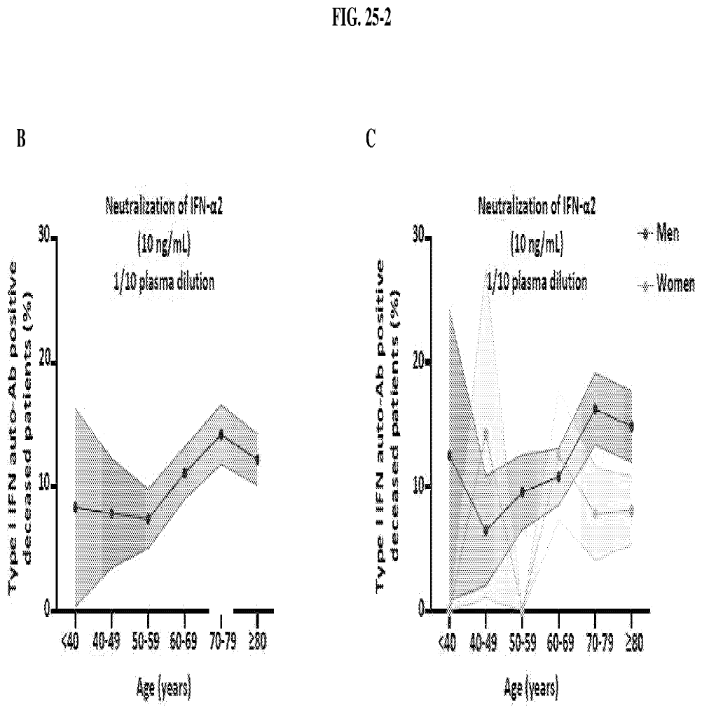

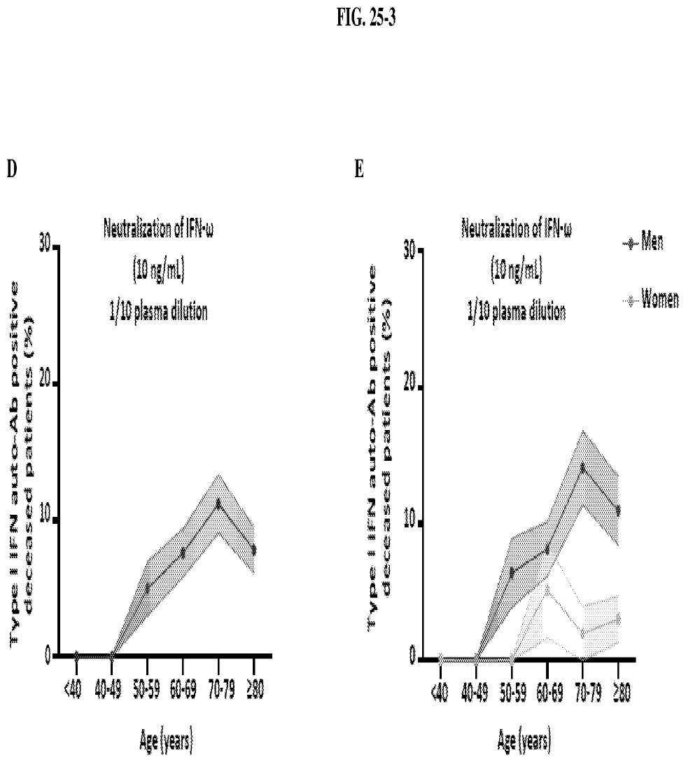

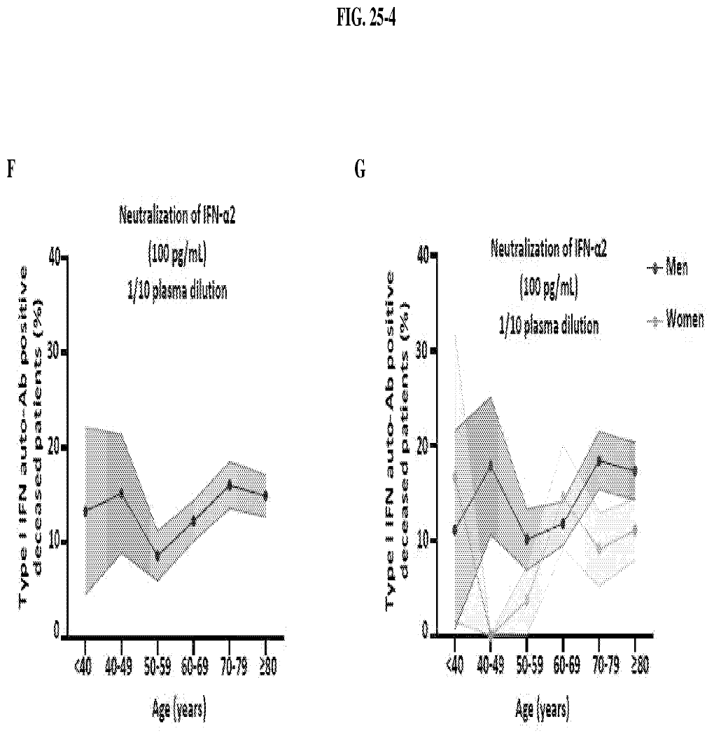

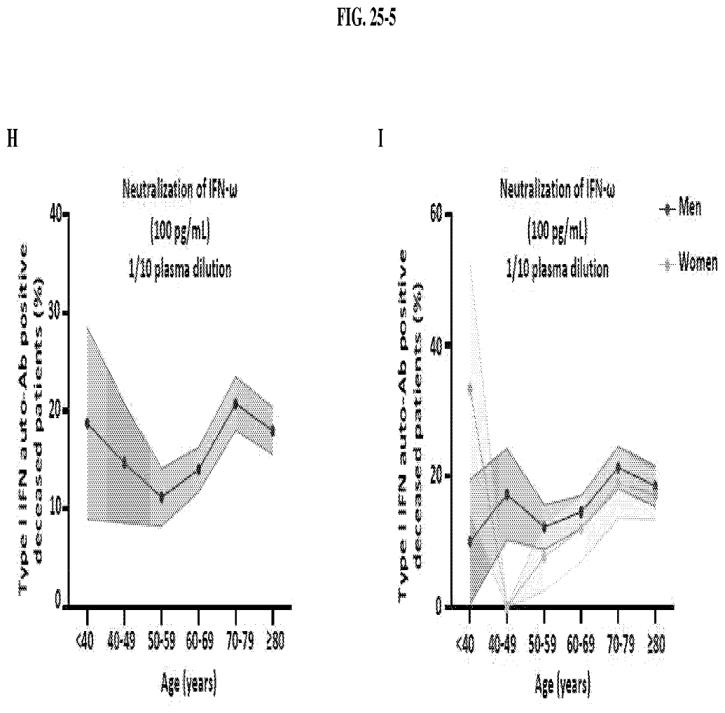

The prevalence of auto-Abs against type I IFNs in patients dying from COVID-19 pneumonia is unknown. We also analyzed a subset of 865 individuals who died from COVID-19 in five different countries (France, Italy, Spain, Turkey, and the United States). These patients were aged 20 to 99 years (mean age: 70 years), 73% were male, and all had confirmed SARS-CoV-2 infection and critical COVID-19 pneumonia before death (FIG. 20A). In these patients, we analyzed the presence of neutralizing auto-Abs against type I IFNs at concentrations of 10 ng/mL and 100 pg/mL (FIG. 20B-J, FIG. 25A-I). We found that 12.5% of the samples from deceased patients carried auto-Abs neutralizing 10 ng/mL IFN-α2 and/or IFN-ω (FIG. 20B-F, FIG. 25A-E). Strikingly, 21% contained auto-Abs neutralizing 100 pg/mL of either or both cytokines (FIG. 20G-J, FIG. 25F-I). An analysis of the prevalence of neutralizing auto-Abs against type I IFNs in these patients who died of COVID-19 by decade of age revealed no significant increase of the prevalence of these auto-Abs with age for auto-Abs neutralizing 10 ng/mL (Wald test, P=0.17) or those neutralizing 100 pg/mL (Wald test, P=0.16). The prevalence of these antibodies exceeded 20% in most age groups (FIG. 20G). For a type I IFN concentration of 100 pg/mL, the prevalence of auto-Abs neutralizing IFN-α2 and/or IFN-ω was 20% below 40 years, 21.2% for individuals between 40 and 49 years old, 13.1% for those between 50 and 60 years old, 20.6% for those between 60 and 69 years old and greater than 22% for those over the age of 70 years. Thus, at least 20% of patients dying from COVID-19 pneumonia have auto-Abs capable of neutralizing 100 pg/mL type I IFNs, in plasma 1/10.

Neutralizing Auto-Abs Against Type I IFNs in 0.2% of Individuals Under the Age of 70 Years

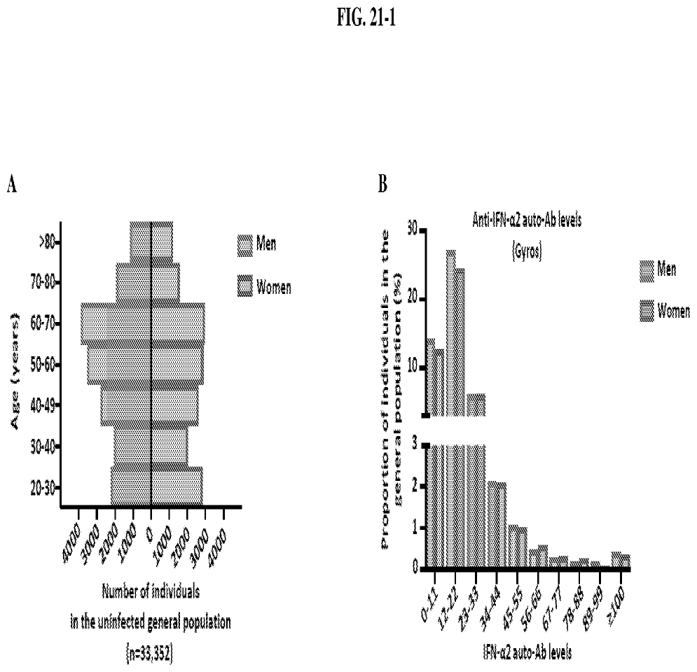

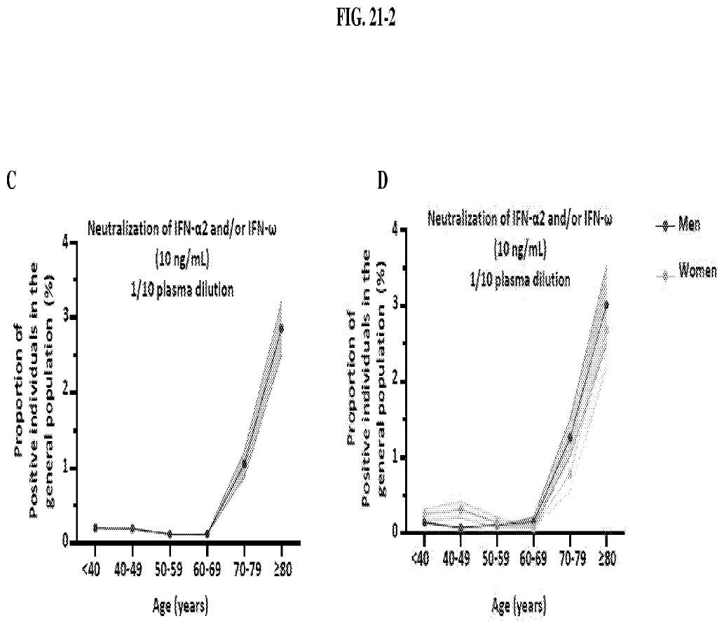

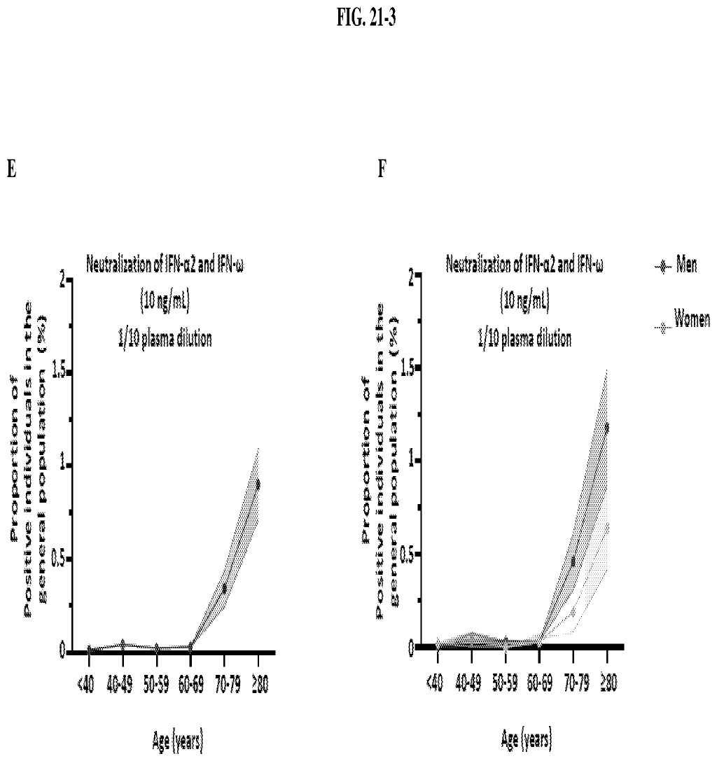

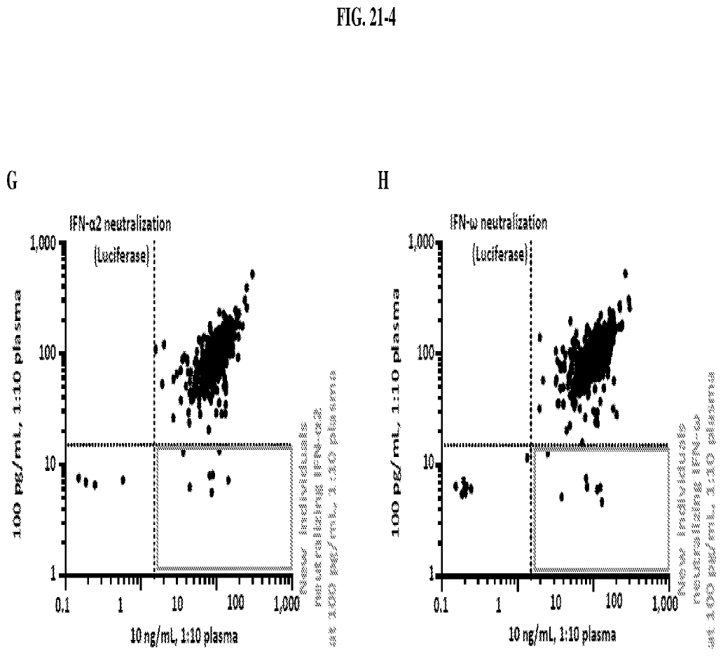

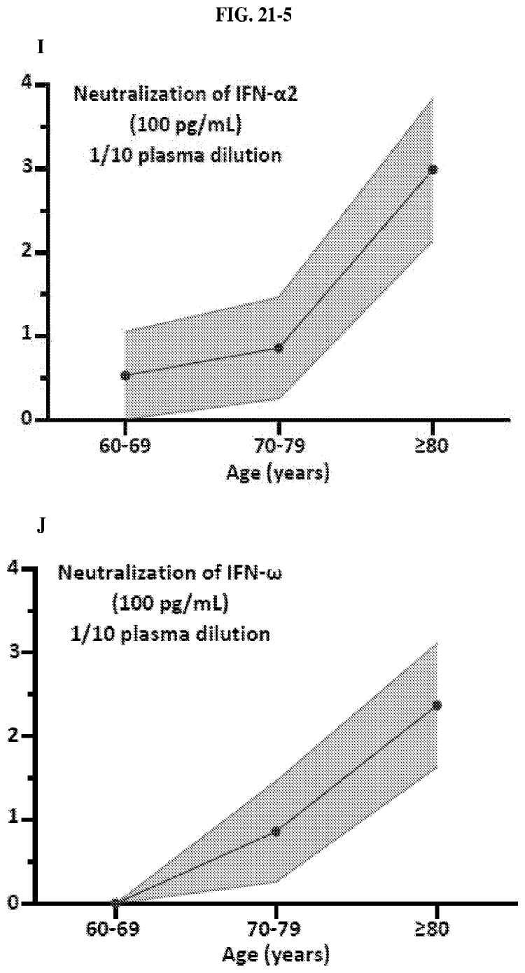

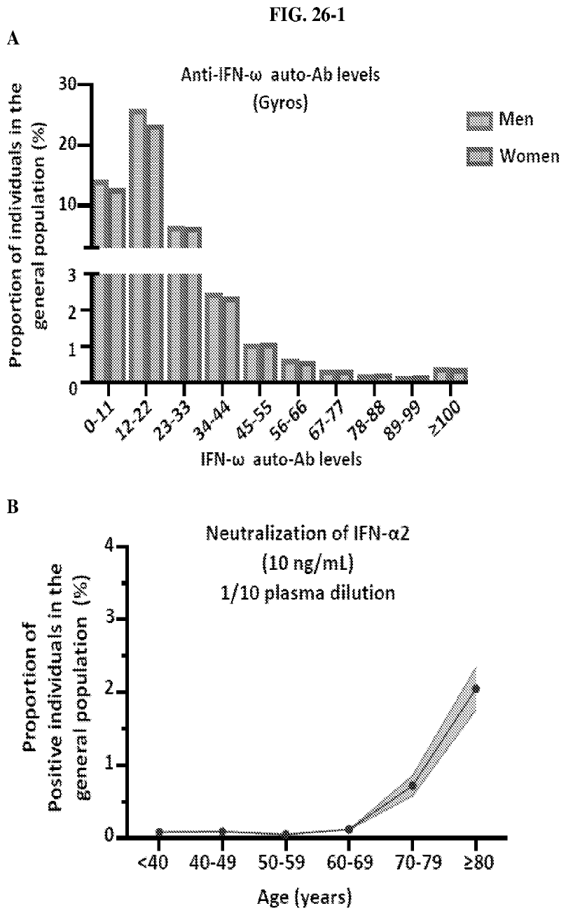

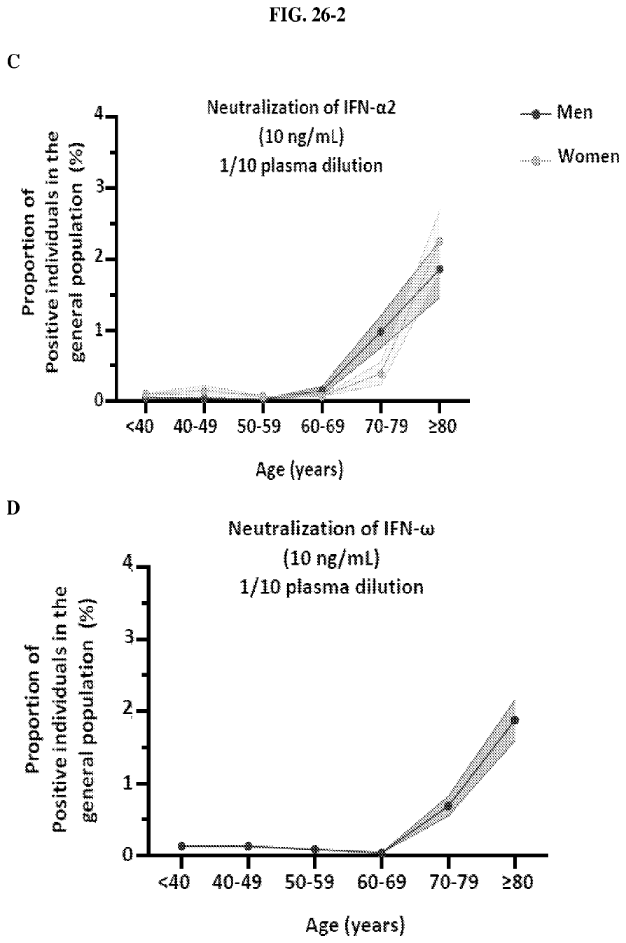

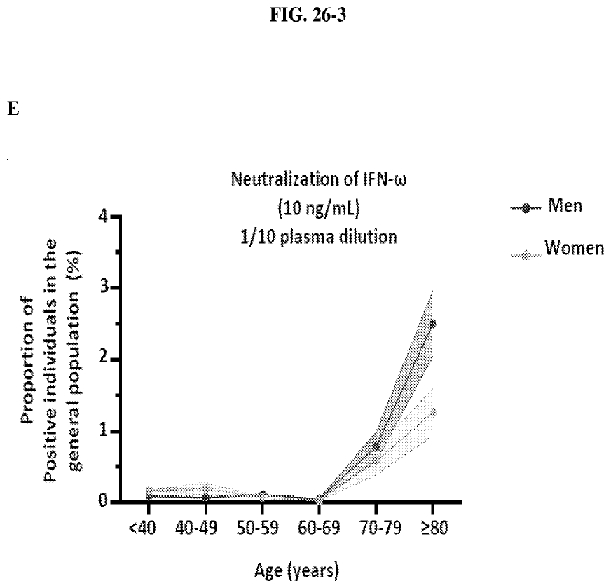

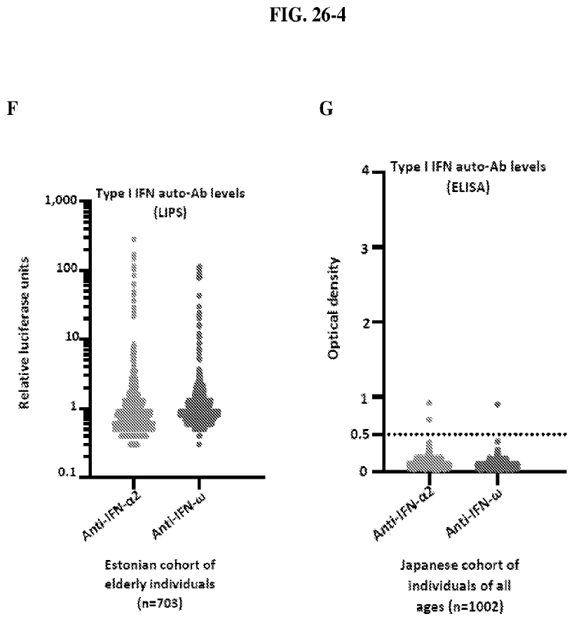

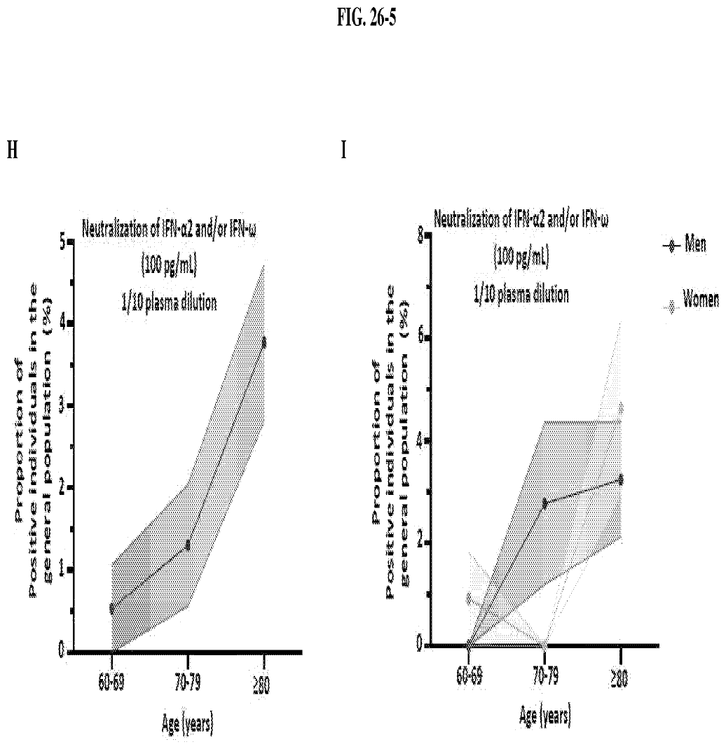

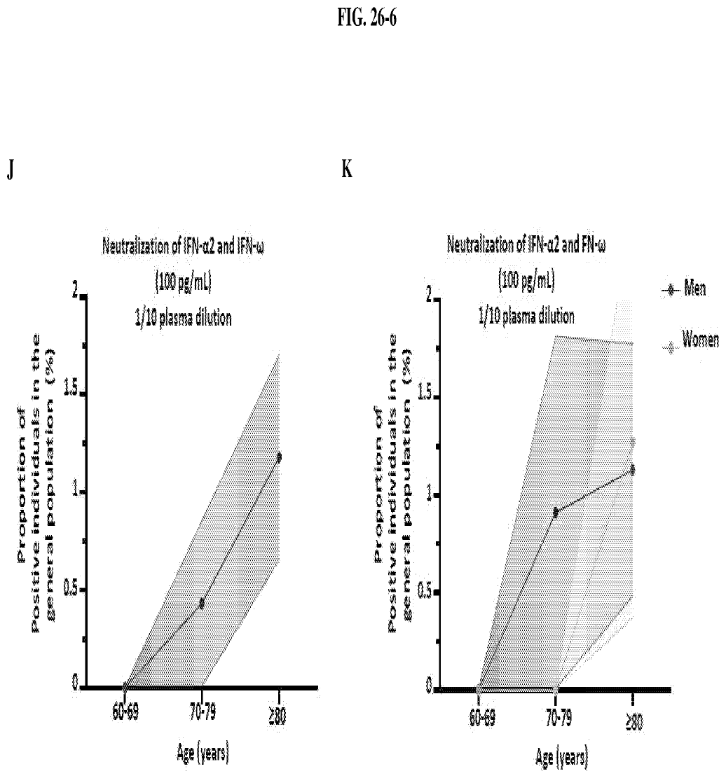

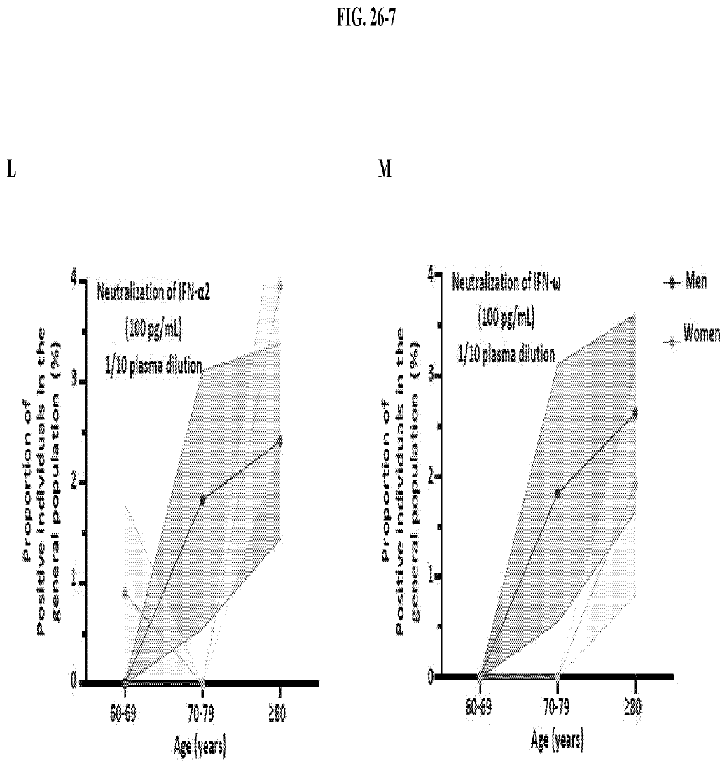

We previously tested a sample of 1,227 individuals aged 20 to 65 years from the general population collected in 2015-2017. This sample had an equal distribution of the two sexes, and we identified four individuals with auto-Abs against type I IFNs among the 1,227 tested (0.3%) (see Example 2; 6). These findings were replicated at the University of California San Francisco (UCSF) in a sample of 4,041 subjects aged four to 90 years (0.2%) (8). We tested a much larger cohort of 27,738 individuals aged 20 to 70 years from the general population, with an equal distribution in both sexes. All samples were collected in 2017, from blood donors at the French Blood Bank (20,093 individuals), participants in the French Constances cohort (5,952), or donors at the US-based IgG producer ADMA pharmaceuticals (1,693) (FIG. 21A). We used Gyros to screen this whole cohort for IgG auto-Abs against IFN-α2 and IFN-ω. We found that only 0.05% and 1.2% had anti-IFN-α2 and/or anti-IFN-ω auto-Abs above the thresholds of 100 and 30, respectively (FIG. 21B, FIG. 26A). We then assessed the ability of these antibodies to neutralize 10 ng/mL IFN-α2 or IFN-ω, for all individuals with a high or intermediate level of IgG auto-Abs against IFN-α2 or IFN-ω. We found 44 individuals with neutralizing auto-Abs, for whom 1/10 dilutions of plasma neutralized 10 ng/mL IFN-α2 and/or IFN-ω, giving an overall prevalence in individuals under the age of 60 years of 0.16% (FIG. 21C-F, FIG. 26B-E) (Table 10), consistent with our two previous reports (6, 8). The prevalence of auto-Abs was not higher in men than in women in this age group, and we instead observed a slightly higher frequency in younger women. In addition, there seemed to be a slightly higher prevalence of neutralizing anti-IFN-ω auto-Abs in younger individuals, especially women (FIG. 26B-E).

Sharp Increase in the Prevalence of Auto-Abs Against Type I IFNs in Subjects Over 70 Years of Age

We assessed the prevalence of auto-Abs against type I IFNs in elderly individuals—the group most affected by critical COVID-19 pneumonia—by recruiting another cohort from the general population, of 5,611 individuals over the age of 70 years, equally distributed between the sexes (FIG. 5A). These individuals were recruited from the 3-C cohort (N=980), the Constances cohort (N=2,631), and Cerba Health Care (N=2,000). We performed serological tests for SARS-CoV-2 on the samples collected after 2019 (N=2,2631), and we included only those individuals who had not been infected with SARS-CoV-2 in the sample. Anti-IFN-α2 and IFN-ω auto-Abs were also tested with Gyros in all samples (FIG. 21B, FIG. 26A). We then assessed in all samples the ability of the plasma/serum samples (diluted 1/10) to neutralize 10 ng/mL and 100 pg/mL type I IFN in the luciferase assay. Strikingly, we noted that the prevalence of auto-Abs neutralizing 10 ng/mL type I IFN was more than 10 times higher in individuals over the age of 70 years than in those below this age (Wald test, P<2·10⁻¹⁶) (FIG. 21C-F, FIG. 26B-E) (Table 10).

TABLE 10 below provides total numbers of patients, controls and individuals from the general population tested for neutralizing auto-Abs against type I IFNs. All is shown by gender, age, type of type I IFN auto-Ab and dose of type I IFN neutralized.

TABLE 10

| | | | Patients with critical COVID-19 | | | Deceased patients | |
|---|---|---|---|---|---|---|---|
| Type \| IFN auto- | | | | | Proportion | | Number of |
| Ab (and | | | | Number of | of patients | | deceased |
| amount of | | | | patients | with | Number | patients |
| type \| IFN | | | | with | neutralizing | of | with |
| neutralized, in | | Age | Number of | neutralizing | auto-Abs | deceased | neutralizing |
| plasma 1/10) | Sex | (years) | patients | auto-Abs | (%) | patients | auto-Abs |
| anti-IFN2 auto- | All | All | 2274 | 249 | 10.95 | 874 | 119 |
| Abs (100 pg/mL) | | | | | | | |
| anti-IFN2 auto- | All | [0,40) | 204 | 14 | 6.86 | 15 | 2 |
| Abs (100 pg/mL) | | | | | | | |
| anti-IFN2 auto- | All | [40,50) | 288 | 29 | 10.07 | 33 | 5 |
| Abs (100 pg/mL) | | | | | | | |
| anti-IFN2 auto- | All | [50,60) | 467 | 38 | 8.14 | 116 | 10 |
| Abs (100 pg/mL) | | | | | | | |
| anti-IFN2 auto- | All | [60,70) | 564 | 62 | 10.99 | 236 | 29 |
| Abs (100 pg/mL) | | | | | | | |
| anti-IFN2 auto- | All | [70,80) | 432 | 60 | 13.89 | 218 | 35 |
| Abs (100 pg/mL) | | | | | | | |
| anti-IFN2 auto- | All | [80,110) | 292 | 46 | 15.75 | 255 | 38 |
| Abs (100 pg/mL) | | | | | | | |
| anti-IFNa2 and | All | All | 2085 | 110 | 5.28 | 368 | 59 |
| anti-IFNw auto- | | | | | | | |
| Abs (10 ng/mL) | | | | | | | |
| anti-IFNa2 and | All | [0,40) | 146 | 1 | 0.68 | 12 | 0 |
| anti-IFNw auto- | | | | | | | |
| Abs (10 ng/mL) | | | | | | | |
| anti-IFNa2 and | All | [40,50) | 244 | 7 | 2.87 | 38 | 0 |
| anti-IFNw auto- | | | | | | | |
| Abs (10 ng/mL) | | | | | | | |
| anti-IFNa2 and | All | [50,60) | 429 | 17 | 3.96 | 121 | 6 |
| anti-IFNw auto- | | | | | | | |
| Abs (10 ng/mL) | | | | | | | |
| anti-IFNa2 and | All | [60,70) | 546 | 28 | 5.13 | 226 | 13 |
| anti-IFNw auto- | | | | | | | |
| Abs (10 ng/mL) | | | | | | | |
| anti-IFNa2 and | All | [70,80) | 404 | 3 | 8.17 | 214 | 21 |
| anti-IFNw auto- | | | | | | | |
| Abs (10 ng/mL) | | | | | | | |
| anti-IFNa2 and | All | [80,110) | 296 | 24 | 8.11 | 256 | 19 |
| anti-IFNw auto- | | | | | | | |
| Abs (10 ng/mL) | | | | | | | |
| anti-IFNa2 and | All | All | 2295 | 170 | 7.41 | 885 | 82 |
| anti-IFNw auto- | | | | | | | |
| Abs (100 pg/mL) | | | | | | | |
| anti-IFNa2 and | All | [0,40) | 205 | 11 | 5.37 | 16 | 2 |
| anti-IFNw auto- | | | | | | | |
| Abs (100 pg/mL) | | | | | | | |
| anti-IFNa2 and | All | [40,50) | 292 | 20 | 6.85 | 34 | 3 |
| anti-IFNw auto- | | | | | | | |
| Abs (100 pg/mL) | | | | | | | |
| anti-IFNa2 and | All | [50,60) | 471 | 22 | 4.67 | 118 | 8 |
| anti-IFNw auto- | | | | | | | |
| Abs (100 pg/mL) | | | | | | | |
| anti-IFNa2 and | Al | [60,70) | 567 | 38 | 6.70 | 238 | 14 |
| anti-IFNw auto- | | | | | | | |
| Abs (100 pg/mL) | | | | | | | |
| anti-IFNa2 and | All | [70,80) | 435 | 45 | 10.34 | 220 | 28 |
| anti-IFNw auto- | | | | | | | |
| Abs (100 pg/mL) | | | | | | | |
| anti-IFNa2 and | All | [80,110) | 297 | 34 | 11.45 | 257 | 27 |
| anti-IFNw auto- | | | | | | | |
| Abs (100 pg/mL) | | | | | | | |
| anti-IFNa2 and/or | All | All | 2036 | 204 | 10.02 | 855 | 107 |
| anti-IFNw auto- | | | | | | | |
| Abs (10 ng/mL) | | | | | | | |
| anti-IFNa2 and/or | All | [0,40) | 146 | 7 | 4.79 | 12 | 1 |
| anti-IFNw auto- | | | | | | | |
| Abs (10 ng/mL) | | | | | | | |
| anti-IFNa2 and/or | All | [40,50) | 239 | 14 | 5.86 | 37 | 3 |
| anti-IFNw auto- | | | | | | | |
| Abs (10 ng/mL) | | | | | | | |
| anti-IFNa2 and/or | All | [50,60) | 419 | 32 | 7.64 | 120 | 9 |
| anti-IFNw auto- | | | | | | | |
| Abs (10 ng/mL) | | | | | | | |
| anti-IFNa2 and/or | All | [60,70) | 528 | 59 | 11.17 | 222 | 29 |
| anti-IFNw auto- | | | | | | | |
| Abs (10 mg/mL) | | | | | | | |
| anti-IFNa2 and/or | All | [70,80) | 394 | 52 | 13.20 | 211 | 33 |
| anti-IFNw auto- | | | | | | | |
| Abs (10 ng/mL) | | | | | | | |
| anti-IFNa2 and/or | All | [80,110) | 291 | 40 | 13.75 | 253 | 32 |
| anti-IFNw auto- | | | | | | | |
| Abs (10 ng/mL) | | | | | | | |
| anti-IFNa2 and/or | All | All | 2248 | 353 | 15.70 | 865 | 182 |
| anti-IFNw auto- | | | | | | | |
| Abs (100 pg/mL) | | | | | | | |
| anti-IFNa2 and/or | All | [0,40) | 202 | 25 | 12.38 | 15 | 3 |
| anti-IFNw auto- | | | | | | | |
| Abs (100 pg/mL) | | | | | | | |
| anti-IFNa2 and/or | All | [40,50) | 286 | 41 | 14.34 | 33 | 7 |
| anti-IFNw auto- | | | | | | | |
| Abs (100 pg/mL) | | | | | | | |
| anti-IFNa2 and/or | All | [50,60) | 461 | 53 | 11.50 | 114 | 15 |
| anti-IFNw auto- | | | | | | | |
| Abs (100 pg/mL) | | | | | | | |
| anti-IFNa2 and/or | All | [60,70) | 556 | 90 | 16.19 | 233 | 48 |
| anti-IFNw auto- | | | | | | | |
| Abs (100 pg/mL) | | | | | | | |
| anti-IFNa2 and/or | All | [70,80) | 427 | 79 | 18.50 | 215 | 52 |
| anti-IFNw auto- | | | | | | | |
| Abs (100 pg/mL) | | | | | | | |
| anti-IFNa2 and/or | All | [80,110) | 290 | 65 | 22.41 | 254 | 57 |
| anti-IFNw auto- | | | | | | | |
| Abs (100 pg/mL) | | | | | | | |
| anti-IFNa2 auto- | All | All | 2066 | 186 | 9.00 | 86* | 99 |
| Abs (10 mg/mL) | | | | | | | |
| anti-IFNa2 auto- | All | [0,40) | 146 | 5 | 3.42 | 12 | 1 |
| Abs (10 ng/mL) | | | | | | | |
| anti-IFNa2 auto- | All | [40,50) | 243 | 12 | 4.94 | 38 | 3 |
| Abs (10 ng/mL) | | | | | | | |
| anti-IFNa2 auto- | All | [50,60) | 427 | 29 | 6.79 | 121 | 9 |
| Abs (10 ng/mL) | | | | | | | |
| anti-IFNa2 auto- | All | [60,70) | 541 | 53 | 9.80 | 225 | 25 |
| Abs (10 ng/mL) | | | | | | | |
| anti-IFNa2 auto- | All | [70,80) | 398 | 48 | 12.06 | 211 | 30 |
| Abs (10 ng/mL) | | | | | | | |
| anti-IFNa2 auto- | All | [80,110) | 292 | 39 | 13.36 | 25 | 31 |
| Abs (10 ng/mL) | | | | | | | |
| anti-IFNw auto- | All | All | 2055 | 128 | 6.23 | 862 | 67 |
| Abs (10 ng/mL) | | | | | | | |
| anti-IFNw auto- | All | [0,40) | 146 | 3 | 2.05 | 12 | 0 |
| Abs (10 ng/mL) | | | | | | | |
| anti-IFNw auto- | All | [40,50) | 240 | 9 | 3.75 | 37 | 0 |
| Abs (10 ng/mL) | | | | | | | |
| anti-IFNw auto- | All | [50,60) | 421 | 20 | 4.75 | 120 | 6 |
| Abs (10 mg/L) | | | | | | | |
| anti-IFNw auto- | All | [60,70) | 533 | 34 | 6.38 | 223 | 17 |
| Abs (10 ng/mL) | | | | | | | |
| anti-IFNw auto- | All | [70,80) | 400 | 37 | 9.25 | 214 | 24 |
| Abs (10 ng/mL) | | | | | | | |
| anti-IFNw auto- | All | [80,110) | 295 | 25 | 8.47 | 255 | 20 |
| Abs (10 mg/mL) | | | | | | | |
| anti-IFNw auto- | All | All | 2269 | 274 | 12.08 | 876 | 145 |
| Abs (100 pg/mL) | | | | | | | |
| anti-IFNw auto- | All | [0,40) | 203 | 22 | 10.84 | 16 | 3 |
| Abs (100 pg/mL) | | | | | | | |
| anti-IFNw auto- | All | [40,50) | 290 | 32 | 11.03 | 34 | 5 |
| Abs (100 pg/mL) | | | | | | | |
| anti-IFNw auto- | All | [50,60) | 465 | 37 | 7.96 | 116 | 13 |
| Abs (100 pg/mL) | | | | | | | |
| anti-IFNw auto- | All | [60,70) | 559 | 66 | 11.81 | 235 | 33 |
| Abs (100 pg/mL) | | | | | | | |
| anti-IFNw auto- | All | [70,80) | 430 | 64 | 14.88 | 217 | 45 |
| Abs (100 pg/mL) | | | | | | | |
| anti-IFNw auto- | All | [80,110) | 295 | 53 | 17.97 | 256 | 46 |
| Abs (100 pg/mL) | | | | | | | |
| only anti-IFNa2 | All | All | 2065 | 75 | 3.63 | 861 | 40 |
| auto-Abs (10 | | | | | | | |
| ng/mL) | | | | | | | |
| only anti-IFNa2 | All | [0,40) | 146 | 4 | 2.74 | 12 | 1 |
| auto-Abs (10 | | | | | | | |
| ng/mL) | | | | | | | |
| only anti-IFNa2 | All | [40,50) | 243 | 5 | 2.06 | 38 | 3 |
| auto-Abs (10 | | | | | | | |
| ng/mL) | | | | | | | |
| only anti-IFNa2 | All | [50,60) | 427 | 12 | 2.81 | 121 | 3 |
| auto-Abs (10 | | | | | | | |
| ng/mL) | | | | | | | |
| only anti-IFNa2 | All | [60,70) | 541 | 25 | 4.62 | 225 | 12 |
| auto-Abs (10 | | | | | | | |
| ng/mL) | | | | | | | |
| only anti-IFNa2 | All | [70,80) | 398 | 15 | 3.77 | 211 | 9 |
| auto-Abs (10 | | | | | | | |
| ng/mL ) | | | | | | | |
| only anti-IFNa2 | All | [80,110) | 291 | 14 | 4.81 | 254 | 12 |
| auto-Abs (10 | | | | | | | |
| ng/mL) | | | | | | | |
| only and-IFNa2 | All | All | 2272 | 77 | 3.39 | 873 | 36 |
| auto-Abs (100 | | | | | | | |
| pg/mL) | | | | | | | |
| only anti-IFNa2 | All | [0,40) | 204 | 3 | 1.47 | 15 | 0 |
| auto-Abs (100 | | | | | | | |
| pg/mL) | | | | | | | |
| only anti-IFNa2 | All | [40,50) | 288 | 9 | 3.13 | 33 | 2 |
| auto-Abs (100 | | | | | | | |
| pg/mL) | | | | | | | |
| only anti-IFNa2 | All | [50,60) | 467 | 16 | 3.43 | 11 | 2 |
| auto-Abs (100 | | | | | | | |
| pg/mL) | | | | | | | |
| only anti-IFNa2 | All | [60,70) | 563 | 23 | 4.09 | 235 | 14 |
| auto-Abs (100 | | | | | | | |
| pg/mL) | | | | | | | |
| only anti-IFNa2 | All | [70,80) | 431 | 14 | 3.25 | 218 | 7 |
| auto-Abs (100 | | | | | | | |
| pg/mL) | | | | | | | |
| only anti-IFNa2 | All | [80,110) | 292 | 12 | 4.11 | 255 | 11 |
| auto-Abs (100 | | | | | | | |
| pg/mL) | | | | | | | |
| only anti-IFNw | All | All | 2054 | 17 | 0.83 | 862 | 8 |
| auto-Abs (10 | | | | | | | |
| ng/mL) | | | | | | | |
| only anti-IFNw | All | [0,40) | 145 | 1 | 0.69 | 12 | 0 |
| auto-Abs (10 | | | | | | | |
| ng/mL) | | | | | | | |
| only anti-IFNw | All | [40,50) | 240 | 2 | 0.83 | 37 | 0 |
| auto-Abs (10 | | | | | | | |
| ng/mL) | | | | | | | |
| only anti-IFNw | All | [50,60) | 421 | 3 | 0.71 | 120 | 0 |
| auto-Abs (10 | | | | | | | |
| ng/mL) | | | | | | | |
| only anti-IFNw | All | [60,70) | 533 | 6 | 1.13 | 223 | 4 |
| auto-Abs (10 | | | | | | | |
| ng/mL) | | | | | | | |
| only anti-IFNw | All | [70,80) | 400 | 4 | 1.00 | 21 | 3 |
| auto-Abs (10 | | | | | | | |
| ng/mL) | | | | | | | |
| only anti-IFNw | All | [80,110) | 295 | 1 | 0.34 | 255 | 1 |
| auto-Abs (10 | | | | | | | |
| ng/mL) | | | | | | | |
| only anti-IFNw | All | All | 2268 | 103 | 4.54 | 876 | 63 |
| auto-Abs (100 | | | | | | | |
| pg/mL) | | | | | | | |
| only anti-IFNw | All | [0,40) | 202 | 10 | 4.95 | 16 | 1 |
| auto-Abs (100 | | | | | | | |
| pg/mL) | | | | | | | |
| only anti-IFNw | All | [40,50) | 290 | 12 | 4.14 | 34 | 2 |
| auto-Abs (100 | | | | | | | |
| pg/mL) | | | | | | | |
| only anti-IFNw | All | [50,60) | 465 | 15 | 3.23 | 116 | 5 |
| auto-Abs (100 | | | | | | | |
| pg/mL) | | | | | | | |
| only anti-IFNw | All | [60,70) | 559 | 28 | 5.01 | 235 | 19 |
| auto-Abs (100 | | | | | | | |
| pg/mL) | | | | | | | |
| only anti-IFNw | All | [70,80) | 430 | 19 | 4.42 | 217 | 17 |
| auto-Abs (100 | | | | | | | |
| pg/mL) | | | | | | | |
| only anti-IFNw | All | [80,110) | 295 | 19 | 6.44 | 256 | 19 |
| auto-Abs (100 | | | | | | | |
| pg/mL) | | | | | | | |
| anti-IFN2 auto- | F | All | 603 | 57 | 9.45 | 234 | 24 |
| Abs (100 pg/mL) | | | | | | | |
| anti-IFN2 auto- | F | [0,40) | 64 | 3 | 4.69 | 6 | 1 |
| Abs (100 pg/mL) | | | | | | | |
| anti-IFN2 auto- | F | [40,50) | 69 | 5 | 7.25 | 5 | 0 |
| Abs (100 pg/mL) | | | | | | | |
| anti-IFN2 auto- | F | [50,60) | 112 | 10 | 8.93 | 27 | 1 |
| Abs (100 pg/mL) | | | | | | | |
| anti-IFN2 auto- | F | [60,70) | 129 | 15 | 11.63 | 41 | 6 |
| Abs (100 pg/mL) | | | | | | | |
| anti-IFN2 auto- | F | [70,80) | 111 | 10 | 9.01 | 55 | 5 |
| Abs (100 pg/mL) | | | | | | | |
| anti-FN2 auto- | F | [80,110) | 115 | 14 | 12.17 | 99 | 11 |
| Abs (100 pg/mL) | | | | | | | |
| anti-IFNa2 and | F | All | 566 | 17 | 3.00 | 230 | 6 |
| anti-IFNw auto- | | | | | | | |
| Abs (10 ng/mL) | | | | | | | |
| anti-IFNa2 and | F | [0,40) | 54 | 0 | 0.00 | 1 | 0 |
| anti-IFNw auto- | | | | | | | |
| Abs (10 ng/mL) | | | | | | | |
| anti-IFNa2 and | F | [40,50) | 59 | 1 | 1.69 | 7 | 0 |
| anti-IFNw auto- | | | | | | | |
| Abs (10 ng/mL) | | | | | | | |
| anti-IFNa2 and | F | [50,60) | 102 | 2 | 1.96 | 27 | 0 |
| anti-IFNw auto- | | | | | | | |
| Abs (10 ng/mL) | | | | | | | |
| anti-IFNa2 and | F | [60,70) | 131 | 6 | 4.58 | 40 | 2 |
| anti-IFNw auto- | | | | | | | |
| Abs (10 ng/mL) | | | | | | | |
| anti-IFNa2 and | F | [70,80) | 102 | 4 | 3.92 | 51 | 1 |
| anti-IFNw auto- | | | | | | | |
| Abs (10 mg/mL) | | | | | | | |
| anti-IFNa2 and | F | [80,110) | 117 | 4 | 3.42 | 100 | 3 |
| anti-IFNw auto- | | | | | | | |
| Abs (10 ng/mL) | | | | | | | |
| anti-FNa2 and | F | All | 610 | 32 | 5.25 | 237 | 15 |
| anti-IFNw auto- | | | | | | | |
| Abs (100 pg/mL) | | | | | | | |
| anti-IFNa2 and | F | [0,40) | 64 | 2 | 3.13 | 6 | 1 |
| anti-IFNw auto- | | | | | | | |
| Abs (100 pg/mL) | | | | | | | |
| anti-IFNa2 and | F | [40,50) | 70 | 2 | 2.86 | 5 | 0 |
| anti-IFNw auto- | | | | | | | |
| Abs (100 pg/mL) | | | | | | | |
| anti-IFNa2 and | F | [50,60) | 113 | 2 | 1.77 | 28 | 0 |
| anti-IFNw auto- | | | | | | | |
| Abs (100 pg/mL] | | | | | | | |
| anti-IFNa2 and | F | [60,70) | 130 | 9 | 6.92 | 42 | 3 |
| anti-IFNw auto- | | | | | | | |
| Abs (100 pg/mL) | | | | | | | |
| anti-IFNa2 and | F | [70,80) | 112 | 7 | 6.25 | 55 | 3 |
| anti-IFNw auto- | | | | | | | |
| Abs (100 pg/mL) | | | | | | | |
| anti-IFNa2 and | F | [80,110) | 117 | 10 | 8.55 | 99 | 8 |
| anti-IFNw auto- | | | | | | | |
| Abs (100 pg/mL) | | | | | | | |
| anti-IFNa2 and/or | F | All | 552 | 46 | 8.33 | 22 | 18 |
| anti-IFNw auto- | | | | | | | |
| Abs (10 ng/mL) | | | | | | | |
| anti-IFNa2 and/or | F | [0,40) | 53 | 1 | .89 | 4 | 0 |
| anti-IFNw auto- | | | | | | | |
| Abs (10 ng/mL) | | | | | | | |
| anti-IFNa2 and/or | F | [40,50) | 58 | 3 | 5.17 | 7 | 1 |
| anti-IFNw auto- | | | | | | | |
| Abs (10 ng/mL) | | | | | | | |
| anti-IFNa2 and/or | F | [50,60) | 100 | 7 | 7.00 | 26 | 0 |
| anti-IFNw auto- | | | | | | | |
| Abs (10 ng/mL) | | | | | | | |
| anti-IFNa2 and/or | F | [60,70) | 125 | 16 | 12.80 | 39 | 5 |
| anti-IFNw auto- | | | | | | | |
| Abs (10 ng/mL) | | | | | | | |
| anti-IFNa2 and/or | F | [70,80) | 100 | 8 | 8.00 | 5 | 4 |
| anti-IFNw auto- | | | | | | | |
| Abs (10 ng/mL) | | | | | | | |
| anti-IFNa2 and/or | F | [80,110) | 116 | 11 | 9.48 | 99 | 8 |
| anti-IFNw auto- | | | | | | | |
| Abs (10 ng/mL) | | | | | | | |
| anti-IFNa2 and/or | F | All | 593 | 91 | 15.35 | 230 | 45 |
| anti-IFNw auto- | | | | | | | |
| Abs (100 pg/mL) | | | | | | | |
| anti-IFNa2 and/or | F | [0,40) | 62 | 8 | 12.90 | 6 | 2 |
| anti-IFNw auto- | | | | | | | |
| Abs (100 pg/mL) | | | | | | | |
| anti-IFNa2 and/or | F | [40,50) | 67 | 8 | 11.94 | 5 | 0 |
| anti-IFNw auto- | | | | | | | |
| Abs (100 pg/mL) | | | | | | | |
| anti-IFNa2 and/or | F | [50,60) | 109 | 15 | 13.76 | 25 | 3 |
| anti-IFNw auto- | | | | | | | |
| Abs (100 pg/mL) | | | | | | | |
| anti-IFNa2 and/or | F | [60,70) | 129 | 19 | 14.73 | 41 | 8 |
| anti-IFNw auto- | | | | | | | |
| Ab (100 pg/mL) | | | | | | | |
| anti-IFNa2 and/or | F | [70,80) | 108 | 18 | 16.67 | 53 | 12 |
| anti-IFNw auto- | | | | | | | |
| Ab (100 pg/mL) | | | | | | | |
| anti-IFNa2 and/or | F | [80,110) | 115 | 23 | 20.00 | 99 | 20 |
| anti-IFNw auto- | | | | | | | |
| Abs (100 pg/mL) | | | | | | | |
| anti-IFNa2 auto- | F | All | 560 | 43 | 7.68 | 228 | 18 |
| Abs (10 ng/mL) | | | | | | | |
| anti-IFNa2 auto- | F | [0,40) | 54 | 1 | 1.85 | 4 | 0 |
| Abs (10 ng/mL) | | | | | | | |
| anti-IFNa2 auto- | F | [40,50) | 58 | 3 | 5.17 | 7 | 1 |
| Abs (10 ng/mL) | | | | | | | |
| anti-IFNa2 auto- | F | [50,60) | 102 | 6 | 5.88 | 27 | 0 |
| Abs (10 ng/mL) | | | | | | | |
| anti-IFNa2 auto- | F | [60,70) | 129 | 15 | 11.63 | 40 | 5 |
| Abs (10 ng/mL) | | | | | | | |
| anti-IFNa2 auto- | F | [70,80) | 101 | 7 | 6.93 | 51 | 4 |
| Abs (10 ng/mL) | | | | | | | |
| anti-IFNa2 auto- | F | [80,110) | 116 | 11 | 9.48 | 99 | 8 |
| Abs (10 ng/mL) | | | | | | | |
| anti-IFNw auto- | F | All | 558 | 20 | 3.58 | 228 | 6 |
| Abs (10 ng/mL) | | | | | | | |
| anti-IFNw auto- | F | [0,40) | 53 | 0 | 0.00 | 4 | 0 |
| Abs (10 ng/mL) | | | | | | | |
| anti-IFNw auto- | F | [40,50) | 59 | 1 | 1.69 | 7 | 0 |
| Abs (10 ng/mL) | | | | | | | |
| anti-IFNw auto- | F | [50,60) | 100 | 3 | 3.00 | 26 | 0 |
| Abs (10 ng/mL) | | | | | | | |
| anti-IFNw auto- | F | [60,70) | 127 | 7 | 5.5 | 39 | 2 |
| Abs (10 ng/mL) | | | | | | | |
| anti-IFNw auto- | F | [70,80) | 101 | 5 | 4.95 | 51 | 1 |
| Abs (10 ng/mL) | | | | | | | |
| anti-IFNw auto- | F | [80,110) | 117 | 4 | 3.42 | 100 | 3 |
| Abs (10 ng/mL) | | | | | | | |
| anti-IFNw auto- | F | All | 600 | 66 | 11.00 | 233 | 36 |
| Abs (100 pg/mL) | | | | | | | |
| anti-IFNw auto- | F | [0,40) | 62 | 7 | 11.29 | 6 | 2 |
| Abs (100 pg/mL) | | | | | | | |
| anti-IFNw auto- | F | [40,50) | 68 | 5 | 7.35 | 5 | 0 |
| Abs (100 pg/mL) | | | | | | | |
| anti-IFNw auto- | F | [50,60) | 110 | 7 | 6.36 | 26 | 2 |
| Abs (100 pg/mL) | | | | | | | |
| anti-IFNw auto- | F | [60,70) | 130 | 13 | 10.00 | 42 | 5 |
| Abs (100 pg/mL) | | | | | | | |
| anti-IFNw auto- | F | [70,80) | 109 | 15 | 13.76 | 53 | 10 |
| Abs (100 pg/mL) | | | | | | | |
| anti-IFNw auto- | F | [80,110) | 117 | 19 | 16.24 | 99 | 17 |
| Abs (100 pg/mL) | | | | | | | |
| only anti-IFNa2 | F | All | 559 | 25 | 4.47 | 228 | 12 |
| auto-Abs (10 | | | | | | | |
| ng/mL) | | | | | | | |
| only anti-IFNa2 | F | [0,40) | 54 | 1 | 1.85 | 4 | 0 |
| auto-Abs (10 | | | | | | | |
| ng/mL) | | | | | | | |
| only anti-IFNa2 | F | [40,50) | 58 | 2 | 3.45 | 7 | 1 |
| auto-Abs (10 | | | | | | | |
| ng/mL) | | | | | | | |
| only anti-IFNa2 | F | [50,60) | 102 | 4 | 3.92 | 27 | 0 |
| auto-Abs (10 | | | | | | | |
| ng/mL) | | | | | | | |
| only anti-IFNa2 | F | [60,70) | 129 | 9 | 6.98 | 40 | 3 |
| auto-Abs (10 | | | | | | | |
| ng/mL) | | | | | | | |
| only anti-IFNa2 | F | [70,80) | 101 | 3 | 2.97 | 51 | 3 |
| auto-Abs (10 | | | | | | | |
| ng/mL) | | | | | | | |
| only anti-IFNa2 | F | [80,110) | 115 | 6 | 5.22 | 99 | 5 |
| auto-Abs (10 | | | | | | | |
| ng/mL) | | | | | | | |
| only anti-IFNa2 | F | All | 603 | 25 | 4.15 | 234 | 9 |
| auto-Abs (100 | | | | | | | |
| pg/mL) | | | | | | | |
| only anti-IFNa2 | F | [0,40) | 64 | 1 | 1.56 | 6 | 0 |
| auto-Abs (100 | | | | | | | |
| pg/mL) | | | | | | | |
| only anti-IFNa2 | F | [40,50) | 69 | 3 | 4.35 | 5 | 0 |
| auto-Abs (100 | | | | | | | |
| pg/mL) | | | | | | | |
| only anti-IFNa2 | F | [50,60) | 112 | 8 | 7.14 | 27 | 1 |
| auto-Abs (100 | | | | | | | |
| pg/mL) | | | | | | | |
| only anti-IFNa2 | F | [60,70) | 129 | 6 | 4.65 | 41 | 3 |
| auto-Abs (100 | | | | | | | |
| pg/mL) | | | | | | | |
| only anti-IFNa2 | F | [70,80) | 111 | 3 | 2.70 | 55 | 2 |
| auto-Abs (100 | | | | | | | |
| pg/mL) | | | | | | | |
| only anti-IFNa2 | F | [80,110) | 115 | 4 | 3.48 | 99 | 3 |
| auto-Abs (100 | | | | | | | |
| pg/mL) | | | | | | | |
| only anti-IFNw | F | All | 558 | 3 | 0.52 | 228 | 0 |
| auto-Abs (10 | | | | | | | |
| ng/mL) | | | | | | | |
| only anti-IFNw | F | [0,40) | 53 | 0 | 0.00 | 4 | 0 |
| auto-Abs (10 | | | | | | | |
| ng/mL) | | | | | | | |
| only anti-IFNw | F | [40,50) | 59 | 0 | 0.00 | 7 | 0 |
| auto-Abs (10 | | | | | | | |
| ng/mL) | | | | | | | |
| only anti-IFNw | F | [50,60) | 100 | 1 | 1.00 | 26 | 0 |
| auto-Abs (10 | | | | | | | |
| ng/mL) | | | | | | | |
| only anti-IFNw | F | [60,70) | 127 | 1 | 0.79 | 39 | 0 |
| auto-Abs (10 | | | | | | | |
| ng/mL) | | | | | | | |
| only anti-IFNw | F | [70,80) | 101 | 1 | 0.99 | 51 | 0 |
| auto-Abs (10 | | | | | | | |
| ng/mL) | | | | | | | |
| only anti-IFNw | F | [80,110) | 117 | 0 | 0.00 | 100 | 0 |
| auto-Abs (10 | | | | | | | |
| ng/mL) | | | | | | | |
| only anti-IFNw | F | All | 600 | 34 | 5.67 | 233 | 21 |
| auto-Abs (100 | | | | | | | |
| pg/mL) | | | | | | | |
| only anti-IFNw | F | [0,40) | 62 | 5 | 8.06 | 6 | 1 |
| auto-Abs (100 | | | | | | | |
| pg/mL) | | | | | | | |
| only anti-IFNw | F | [40,50) | 68 | 3 | 4.41 | 5 | 0 |
| auto-Abs (100 | | | | | | | |
| pg/mL) | | | | | | | |
| only anti-IFNw | F | [50,60) | 110 | 5 | 4.55 | 26 | 2 |
| auto-Abs (100 | | | | | | | |
| pg/mL) | | | | | | | |
| only anti-IFNw | F | [60,70) | 130 | 4 | 3.08 | 42 | 2 |
| auto-Abs (100 | | | | | | | |
| pg/mL) | | | | | | | |
| only anti-IFNw | F | [70,80) | 109 | 8 | 7.34 | 53 | 7 |
| auto-Abs (100 | | | | | | | |
| pg/mL) | | | | | | | |
| only anti-IFNw | F | [80,110) | 117 | 9 | 7.69 | 99 | 9 |
| auto-Abs (100 | | | | | | | |
| pg/mL) | | | | | | | |
| anti-IFN2 auto- | M | All | 1644 | 192 | 11.68 | 640 | 95 |
| Abs (100 pg/mL) | | | | | | | |
| anti-IFN2 auto- | M | [0,40) | 138 | 11 | 7.97 | 9 | 1 |
| Abs (100 pg/mL) | | | | | | | |
| anti-IFN2 auto- | M | [40,50) | 218 | 24 | 11.01 | 28 | 5 |
| Abs (100 pg/mL) | | | | | | | |
| anti-IFN2 auto- | M | [50,60) | 353 | 28 | 7.93 | 89 | 9 |
| Abs (100 pg/mL) | | | | | | | |
| anti-IFN2 auto- | M | [60,70) | 435 | 47 | 10.80 | 195 | 23 |
| Abs (100 pg/mL) | | | | | | | |
| anti-IFN2 auto- | M | [70,80) | 320 | 50 | 15.63 | 163 | 30 |
| Abs (100 pg/mL) | | | | | | | |
| anti-IFN2 auto- | M | [80,110) | 177 | 32 | 18.08 | 156 | 27 |
| Abs (100 pg/mL) | | | | | | | |
| anti-IFNa2 and | M | All | 1495 | 93 | 2 | 638 | 53 |
| anti-IFNw auto- | | | | | | | |
| Abs (10 ng/mL) | | | | | | | |
| anti-IFNa2 and | M | [0,40) | 90 | 1 | 1.11 | 8 | 0 |
| anti-IFNw auto- | | | | | | | |
| Abs (10 ng/mL) | | | | | | | |
| anti-IFNa2 and | M | [40,50) | 185 | 6 | 3.24 | 31 | 0 |
| anti-IFNw auto- | | | | | | | |
| Abs (10 ng/mL) | | | | | | | |
| anti-IFNa2 and | M | [50,60) | 325 | 15 | 4.62 | 94 | 6 |
| anti-IFNw auto- | | | | | | | |
| Abs (10 ng/mL) | | | | | | | |
| anti-IFNa2 and | M | [60,70) | 415 | 22 | 5.30 | 186 | 11 |
| anti-IFNw auto- | | | | | | | |
| Abs (10 ng/mL) | | | | | | | |
| anti-IFNa2 and | M | [70,80) | 301 | 29 | 9.63 | 163 | 20 |
| anti-IFNw auto- | | | | | | | |
| Abs (10 ng/mL) | | | | | | | |
| anti-IFNa2 and | M | [80,110) | 179 | 20 | 11.17 | 156 | 16 |
| anti-IFNw auto- | | | | | | | |
| Abs (10 ng/mL) | | | | | | | |
| anti-IFNa2 and | M | All | 1658 | 138 | 8.32 | 648 | 67 |
| anti-IFNw auto- | | | | | | | |
| Abs (100 pg/mL) | | | | | | | |
| anti-IFNa2 and | M | [0,40) | 139 | 9 | 6.47 | 10 | 1 |
| anti-IFNw auto- | | | | | | | |
| Abs (100 pg/mL) | | | | | | | |
| anti-IFNa2 and | M | [40,50) | 221 | 18 | 8.14 | 29 | 3 |
| anti-IFNw auto- | | | | | | | |
| Abs (100 pg/mL) | | | | | | | |
| anti-IFNa2 and | M | [50,60) | 356 | 20 | 5.62 | 90 | 8 |
| anti-IFNw auto- | | | | | | | |
| Abs (100 pg/mL) | | | | | | | |
| anti-IFNa2 and | M | [60,70) | 437 | 29 | 6.64 | 96 | 11 |
| anti-IFNw auto- | | | | | | | |
| Abs (100 pg/mL) | | | | | | | |
| anti-IFNa2 and | M | [70,80) | 322 | 38 | 11.80 | 165 | 25 |
| anti-IFNw auto- | | | | | | | |
| Abs (100 pg/mL) | | | | | | | |
| anti-IFNa2 and | M | [80,110) | 180 | 24 | 13.33 | 158 | 19 |
| anti-IFNw auto- | | | | | | | |
| Abs (100 pg/mL) | | | | | | | |
| anti-IFNa2 and/or | M | All | 1460 | 158 | 10.82 | 629 | 89 |
| anti-IFNw auto- | | | | | | | |
| Abs (10 ng/mL) | | | | | | | |
| anti-IFNa2 and/or | M | [0,40) | 91 | 6 | 6.59 | 8 | 1 |
| anti-IFNw auto- | | | | | | | |
| Abs (10 ng/mL) | | | | | | | |
| anti-IFNa2 and/or | M | [40,50) | 181 | 11 | 6.08 | 30 | 2 |
| anti-IFNw auto- | | | | | | | |
| Abs (10 ng/mL) | | | | | | | |
| anti-IFNa2 and/or | M | [50,60) | 317 | 25 | 7.89 | 94 | 9 |
| anti-IFNw auto- | | | | | | | |
| Abs (10 ng/mL) | | | | | | | |
| anti-IFNa2 and/or | M | [60,70) | 403 | 43 | 10.67 | 183 | 24 |
| anti-IFNw auto- | | | | | | | |
| Abs (10 ng/mL) | | | | | | | |
| anti-IFNa2 and/or | M | [70,80) | 293 | 44 | 15.02 | 160 | 29 |
| anti-IFNw auto- | | | | | | | |
| Abs (10 ng/mL) | | | | | | | |
| anti-IFNa2 and/or | M | [80,110) | 175 | 29 | 16.57 | 154 | 24 |
| anti-IFNw auto- | | | | | | | |
| Abs (10 mg/mL) | | | | | | | |
| anti-IFNa2 and/or | M | All | 1629 | 262 | 16.08 | 635 | 137 |
| anti-IFNw auto- | | | | | | | |
| Abs (100 pg/mL) | | | | | | | |
| anti-IFNa2 and/or | M | [0,40) | 138 | 17 | 12.32 | 9 | 1 |
| anti-IFNw auto- | | | | | | | |
| Abs (100 pg/mL) | | | | | | | |
| anti-IFNa2 and/or | M | [40,50) | 218 | 33 | 15.14 | 28 | 7 |
| anti-IFNw auto- | | | | | | | |
| Abs (100 pg/mL) | | | | | | | |
| anti-IFNa2 and/or | M | [50,60) | 350 | 38 | 10.86 | 89 | 12 |
| anti-IFNw auto- | | | | | | | |
| Abs (100 pg/mL) | | | | | | | |
| anti-IFNa2 and/or | M | [60,70) | 427 | 71 | 16.63 | 192 | 40 |
| anti-IFNw auto- | | | | | | | |
| Abs (100 pg/mL) | | | | | | | |
| anti-IFNa2 and/or | M | [70,80) | 318 | 61 | 19.18 | 162 | 40 |
| anti-IFNw auto- | | | | | | | |
| Abs (100 pg/mL) | | | | | | | |
| anti-IFNa2 and/or | M | [80,110) | 175 | 42 | 24.00 | 155 | 37 |
| anti-IFNw auto- | | | | | | | |
| Abs (100 pg/mL) | | | | | | | |
| anti-IFNa2 auto- | M | All | 1482 | 143 | 9.65 | 633 | 81 |
| Abs (10 ng/mL) | | | | | | | |
| anti-IFNa2 auto- | M | [0,40) | 90 | 4 | 4.44 | 8 | 1 |
| Abs (10 ng/mL) | | | | | | | |
| anti-IFNa2 auto- | M | [40,50) | 185 | 9 | 4.86 | 31 | 2 |
| Abs (10 ng/mL) | | | | | | | |
| anti-IFNa2 auto- | M | [50,60) | 323 | 23 | 7.12 | 94 | 9 |
| Abs (10 ng/mL) | | | | | | | |
| anti-IFNa2 auto- | M | [60,70) | 412 | 38 | 9.22 | 185 | 20 |
| Abs (10 ng/mL) | | | | | | | |
| anti-IFNa2 auto- | M | [70,80) | 296 | 41 | 13.85 | 160 | 26 |
| Abs (10 ng/mL) | | | | | | | |
| anti-IFNa2 auto- | M | [80,110) | 176 | 28 | 15.91 | 155 | 23 |
| Abs (10 ng/mL) | | | | | | | |
| anti-IFNw auto- | M | All | 1473 | 108 | 7.33 | 634 | 61 |
| Abs (10 ng/mL) | | | | | | | |
| anti-IFNw auto- | M | [0,40) | 91 | 3 | 3.30 | 8 | 0 |
| Abs (10 ng/mL) | | | | | | | |
| anti-IFNw auto- | M | [40,50) | 181 | 8 | 4.42 | 30 | 0 |
| Abs (10 ng/mL) | | | | | | | |
| anti-IFNw auto- | M | [50,60) | 319 | 17 | 5.33 | 94 | 6 |
| Abs (10 ng/mL) | | | | | | | |
| anti-IFNw auto- | M | [60,70) | 406 | 27 | 6.65 | 184 | 15 |
| Abs (10 ng/mL) | | | | | | | |
| anti-IFNw auto- | M | [70,80) | 298 | 32 | 10.74 | 163 | 23 |
| Abs (10 ng/mL) | | | | | | | |
| anti-IFNw auto- | M | [80,110) | 178 | 21 | 11.80 | 155 | 17 |
| Abs (10 ng/mL) | | | | | | | |
| anti-IFNw auto- | M | All | 1643 | 208 | 12.66 | 643 | 109 |
| Abs (100 pg/mL) | | | | | | | |
| anti-IFNw auto- | M | [0,40) | 139 | 15 | 10.79 | 10 | 1 |
| Abs (100 pg/mL) | | | | | | | |
| anti-IFNw auto- | M | [40,50) | 221 | 27 | 12.22 | 29 | 5 |
| Abs (100 pg/mL) | | | | | | | |
| anti-IFNw auto- | M | [50,60) | 353 | 30 | 8.50 | 90 | 11 |
| Abs (100 pg/mL) | | | | | | | |
| anti-IFNw auto- | M | [60,70) | 429 | 53 | 12.35 | 193 | 28 |
| Abs (100 pg/mL) | | | | | | | |
| anti-IFNw auto- | M | [70,80) | 320 | 49 | 15.31 | 164 | 35 |
| Abs (100 pg/mL) | | | | | | | |
| anti-IFNw auto- | M | [80,110) | 178 | 34 | 19.10 | 157 | 29 |
| Abs (100 pg/mL) | | | | | | | |
| only anti-IFNa2 | M | All | 1482 | 50 | 3.37 | 633 | 28 |
| auto-Abs (10 | | | | | | | |
| ng/mL) | | | | | | | |
| only anti-IFNa2 | M | [0,40) | 90 | 3 | 3.33 | 8 | 1 |
| auto-Abs (10 | | | | | | | |
| ng/mL) | | | | | | | |
| only anti-IFNa2 | M | [40,50) | 185 | 3 | 1.62 | 31 | 2 |
| auto-Abs (10 | | | | | | | |
| ng/mL) | | | | | | | |
| only anti-IFNa2 | M | [50,60) | 323 | 8 | 2.48 | 94 | 3 |
| auto-Abs (10 | | | | | | | |
| ng/mL) | | | | | | | |
| only anti-IFNa2 | M | [60,70) | 412 | 16 | 3.88 | 185 | 6 |
| auto-Abs (10 | | | | | | | |
| ng/mL) | | | | | | | |
| only anti-IFNa2 | M | [70,80) | 296 | 12 | 4.05 | 160 | 6 |
| auto-Abs (10 | | | | | | | |
| ng/mL) | | | | | | | |
| only anti-IFNa2 | M | [80,110) | 176 | 8 | 4.55 | 155 | 7 |
| auto-Abs (10 | | | | | | | |
| ng/mL) | | | | | | | |
| only anti-IFNa2 | M | All | 1642 | 52 | 3.1 | 639 | 27 |
| auto-Abs (100 | | | | | | | |
| pg/mL) | | | | | | | |
| only anti-IFNa2 | M | [0,40) | 138 | 2 | 1.45 | 9 | 0 |
| auto-Abs (100 | | | | | | | |
| pg/mL) | | | | | | | |
| only anti-IFNa2 | M | [40,50) | 218 | 6 | 2.75 | 28 | 2 |
| auto-Abs (100 | | | | | | | |
| pg/mL) | | | | | | | |
| only anti-IFNa2 | M | [50,60) | 353 | 8 | 2.27 | 89 | 1 |
| auto-Abs (100 | | | | | | | |
| pg/mL) | | | | | | | |
| only anti-IFNa2 | M | [60,70) | 434 | 17 | 3.92 | 194 | 11 |
| auto-Abs (100 | | | | | | | |
| pg/mL) | | | | | | | |
| only anti-IFNa2 | M | [70,80) | 319 | 11 | 3.45 | 163 | 5 |
| auto-Abs (100 | | | | | | | |
| pg/mL) | | | | | | | |
| only anti-IFNa2 | M | [80,110) | 177 | 8 | 4.52 | 156 | 8 |
| auto-Abs (100 | | | | | | | |
| pg/mL) | | | | | | | |
| only anti-IFNw | M | All | 1472 | 14 | 0.95 | 634 | 8 |
| auto-Abs (10 | | | | | | | |
| ng/mL) | | | | | | | |
| only anti-IFNw | M | [0,40) | 90 | 1 | 1.11 | 8 | 0 |
| auto-Abs (10 | | | | | | | |
| ng/mL) | | | | | | | |
| only anti-IFNw | M | [40,50) | 181 | 2 | 1.10 | 30 | 0 |
| auto-Abs (10 | | | | | | | |
| ng/mL) | | | | | | | |
| only anti-IFNw | M | [50,60) | 319 | 2 | 0.63 | 94 | 0 |
| auto-Abs (10 | | | | | | | |
| ng/mL) | | | | | | | |
| only anti-IFNw | M | [60,70) | 406 | 5 | 1,23 | 184 | 4 |
| auto-Abs (10 | | | | | | | |
| ng/mL) | | | | | | | |
| only anti-IFNw | M | [70,80) | 298 | 3 | 1.01 | 163 | 3 |
| auto-Abs (10 | | | | | | | |
| ng/mL) | | | | | | | |
| only anti-IFNw | M | [80,110) | 178 | 1 | 0.56 | 155 | 1 |
| auto-Abs (10 | | | | | | | |
| ng/mL) | | | | | | | |
| only anti-IFNw | M | All | 1642 | 69 | 4.20 | 643 | 42 |
| auto-Abs (100 | | | | | | | |
| pg/mL) | | | | | | | |
| only anti-IFNw | M | [0,40) | 138 | 5 | 3.62 | 10 | 0 |
| auto-Abs (100 | | | | | | | |
| 2g/mL) | | | | | | | |
| only anti-IFNw | M | [40,50) | 221 | 9 | 4.07 | 29 | 2 |
| auto-Abs (100 | | | | | | | |
| pg/mL) | | | | | | | |
| only anti-IFNw | M | [50,60] | 353 | 10 | 2.83 | 90 | 3 |
| auto-Abs (100 | | | | | | | |
| pg/mL) | | | | | | | |
| only anti-IFNw | M | [60,70) | 429 | 24 | 5.59 | 193 | 17 |
| auto-Abs (100 | | | | | | | |
| pg/mL) | | | | | | | |
| only anti-IFNw | M | [70,80) | 320 | 11 | 3.44 | 164 | 10 |
| auto-Abs (100 | | | | | | | |
| pg/mL) | | | | | | | |
| only anti-IFNw | M | [80,110) | 178 | 10 | 5.62 | 15 | 10 |
| auto-Abs (100 | | | | | | | |
| pg/mL) | | | | | | | |
| | | | | | Individuals from the general | | |
| | | | | | population | | |
| | Deceased | | | | | | Proportion |
| | patients | | | | | | of |
| | Proportion | Asymptomatic or | | | | Number of | individuals |
| | of | paucisymptomatic individuals | | | | individual | from the |
| Type \| IFN auto- | deceased | | | Proportion | | from the | general |
| Ab [and | patients | | Number of | of controls | Number of | general | population |
| amount of | with | | controls | with | individuals | population | with |
| type \| IFN | neutralizing | Number | with | neutralizing | from the | with | neutralizing |
| neutralized, in | auto-Abs | of | neutralizing | auto-Abs | general | neutralizing | auto-Abs |
| plasma 1/10) | (%) | controls | auto-Abs | (%) | population | auto-Abs | [%) |
| anti-IFN2 auto- | 13.62 | 612 | 0 | 0.00 | 823 | 15 | 1.82 |
| Abs (100 pg/mL) | | | | | | | |
| anti-IFN2 auto- | 13.33 | 273 | 0 | 0.00 | 0 | 0 | 0.00 |
| Abs (100 pg/mL) | | | | | | | |
| anti-IFN2 auto- | 15.15 | 154 | 0 | 0.00 | 0 | 0 | 0.00 |
| Abs (100 pg/mL) | | | | | | | |
| anti-IFN2 auto- | 8.62 | 138 | 0 | 0.00 | 0 | 0 | 0.00 |
| Abs (100 pg/mL) | | | | | | | |
| anti-IFN2 auto- | 12.29 | 31 | 0 | 0.00 | 190 | 1 | 0.53 |
| Abs (100 pg/mL) | | | | | | | |
| anti-IFN2 auto- | 16.06 | 3 | 0 | 0.00 | 232 | 2 | 0.86 |
| Abs (100 pg/mL) | | | | | | | |
| anti-IFN2 auto- | 14.90 | 5 | 0 | 0.00 | 401 | 12 | 2.99 |
| Abs (100 pg/mL) | | | | | | | |
| anti-IFNa2 and | 6.80 | 343 | 0 | 0.00 | 33821 | 40 | 0.12 |
| anti-IFNw auto- | | | | | | | |
| Abs (10 ng/mL) | | | | | | | |
| anti-IFNa2 and | 0.00 | 118 | 0 | 1.00 | 9110 | 1 | 0.01 |
| anti-IFNw auto- | | | | | | | |
| Abs (10 ng/mL) | | | | | | | |
| anti-IFNa2 and | 0.00 | 102 | 0 | 0.00 | 5406 | 2 | 0.04 |
| anti-IFNw auto- | | | | | | | |
| Abs (10 ng/mL) | | | | | | | |
| anti-IFNa2 and | 4.96 | 85 | 0 | 0.00 | 6422 | 1 | 0.02 |
| anti-IFNw auto- | | | | | | | |
| Abs (10 ng/mL) | | | | | | | |
| anti-IFNa2 and | 5.75 | 25 | 0 | 0.00 | 6889 | 2 | 0.03 |
| anti-IFNw auto- | | | | | | | |
| Abs (10 ng/mL) | | | | | | | |
| anti-IFNa2 and | 9.81 | 3 | 0 | 0.00 | 3542 | 12 | 0.34 |
| anti-IFNw auto- | | | | | | | |
| Abs (10 ng/mL) | | | | | | | |
| anti-IFNa2 and | 7.42 | 5 | 0 | 1.00 | 2449 | 22 | 0.90 |
| anti-IFNw auto- | | | | | | | |
| Abs (10 ng/mL) | | | | | | | |
| anti-IFNa2 and | 9.27 | 638 | 0 | 0.00 | 849 | 6 | 0.71 |
| anti-IFNw auto- | | | | | | | |
| Abs (100 pg/mL) | | | | | | | |
| anti-IFNa2 and | 12.50 | 293 | 0 | 0.00 | 0 | 0 | 0.00 |
| anti-IFNw auto- | | | | | | | |
| Abs (100 pg/mL) | | | | | | | |
| anti-IFNa2 and | 8.82 | 159 | 0 | 1.00 | 0 | 0 | 0.00 |
| anti-IFNw auto- | | | | | | | |
| Abs (100 pg/mL) | | | | | | | |
| anti-IFNa2 and | 6.78 | 139 | 0 | 0.00 | 0 | 0 | 0.00 |
| anti-IFNw auto- | | | | | | | |
| Abs (100 pg/mL) | | | | | | | |
| anti-IFNa2 and | 5.88 | 31 | 0 | 0.00 | 192 | 0 | 0.00 |
| anti-IFNw auto- | | | | | | | |
| Abs (100 pg/mL) | | | | | | | |
| anti-IFNa2 and | 12.73 | 3 | 0 | 0.00 | 234 | 1 | 0.43 |
| anti-IFNw auto- | | | | | | | |
| Abs (100 pg/mL) | | | | | | | |
| anti-IFNa2 and | 10.51 | 5 | 0 | 1.00 | 423 | 5 | 1.18 |
| anti-IFNw auto- | | | | | | | |
| Abs (100 pg/mL) | | | | | | | |
| anti-IFNa2 and/or | 12.51 | 342 | 2 | 0.58 | 33352 | 142 | 0.43 |
| anti-IFNw auto- | | | | | | | |
| Abs (10 ng/mL) | | | | | | | |
| anti-IFNa2 and/or | 8.33 | 118 | 0 | 0.00 | 9102 | 18 | 0.20 |
| anti-IFNw auto- | | | | | | | |
| Abs (10 ng/mL) | | | | | | | |
| anti-IFNa2 and/or | 8.11 | 101 | 1 | 0.99 | 5403 | 10 | 0.19 |
| anti-IFNw auto- | | | | | | | |
| Abs (10 ng/mL) | | | | | | | |
| anti-IFNa2 and/or | 7.50 | 85 | 0 | 0.00 | 6414 | 8 | 0.12 |
| anti-IFNw auto- | | | | | | | |
| Abs (10 ng/mL) | | | | | | | |
| anti-IFNa2 and/or | 13.06 | 25 | 1 | 4.00 | 6819 | 8 | 0.12 |
| anti-IFNw auto- | | | | | | | |
| Abs (10 mg/mL) | | | | | | | |
| anti-IFNa2 and/or | 15.64 | 3 | 0 | 0.00 | 3433 | 36 | 1.05 |
| anti-IFNw auto- | | | | | | | |
| Abs (10 ng/mL) | | | | | | | |
| anti-IFNa2 and/or | 12.65 | 5 | 0 | 0.00 | 2178 | 62 | 2.85 |
| anti-IFNw auto- | | | | | | | |
| Abs (10 ng/mL) | | | | | | | |
| anti-IFNa2 and/or | 21.04 | 588 | 5 | 0.85 | 816 | 19 | 2.33 |
| anti-IFNw auto- | | | | | | | |
| Abs (100 pg/mL) | | | | | | | |
| anti-IFNa2 and/or | 20.00 | 252 | 2 | 0.79 | 0 | 0 | 0.00 |
| anti-IFNw auto- | | | | | | | |
| Abs (100 pg/mL) | | | | | | | |
| anti-IFNa2 and/or | 21.21 | 153 | 1 | 0.65 | 0 | 0 | 0.00 |
| anti-IFNw auto- | | | | | | | |
| Abs (100 pg/mL) | | | | | | | |
| anti-IFNa2 and/or | 13.16 | 136 | 1 | 0.74 | 0 | 0 | 0.00 |
| anti-IFNw auto- | | | | | | | |
| Abs (100 pg/mL) | | | | | | | |
| anti-IFNa2 and/or | 20.60 | 3 | 1 | 3.23 | 187 | 1 | 0.53 |
| anti-IFNw auto- | | | | | | | |
| Abs (100 pg/mL) | | | | | | | |
| anti-IFNa2 and/or | 24.19 | 3 | 0 | 0.00 | 231 | 3 | 1.30 |
| anti-IFNw auto- | | | | | | | |
| Abs (100 pg/mL) | | | | | | | |
| anti-IFNa2 and/or | 22.44 | 5 | 0 | 0.00 | 39 | 15 | 3.77 |
| anti-IFNw auto- | | | | | | | |
| Abs (100 pg/mL) | | | | | | | |
| anti-IFNa2 auto- | 11.50 | 343 | 0 | 0.00 | 33579 | 95 | 0.28 |
| Abs (10 mg/mL) | | | | | | | |
| anti-IFNa2 auto- | 8.33 | 118 | 0 | 0.00 | 9106 | 7 | 0.08 |
| Abs (10 ng/mL) | | | | | | | |
| anti-IFNa2 auto- | 7.89 | 102 | 0 | 0.00 | 5404 | 5 | 0.09 |
| Abs (10 ng/mL) | | | | | | | |
| anti-IFNa2 auto- | 7.44 | 85 | 0 | 0.00 | 6417 | 3 | 0.05 |
| Abs (10 ng/mL) | | | | | | | |
| anti-IFNa2 auto- | 11.11 | 25 | 0 | 0.00 | 6878 | 8 | 0.12 |
| Abs (10 ng/mL) | | | | | | | |
| anti-IFNa2 auto- | 14.22 | 3 | 0 | 0.00 | 3483 | 25 | 0.72 |
| Abs (10 ng/mL) | | | | | | | |
| anti-IFNa2 auto- | 12.20 | 5 | 0 | 0.00 | 2288 | 47 | 2.05 |
| Abs (10 ng/mL) | | | | | | | |
| anti-IFNw auto- | 7.77 | 342 | 2 | 0.58 | 33603 | 96 | 0.29 |
| Abs (10 ng/mL) | | | | | | | |
| anti-IFNw auto- | 0.00 | 118 | 0 | 0.00 | 9106 | 12 | 0.13 |
| Abs (10 ng/mL) | | | | | | | |
| anti-IFNw auto- | 0.00 | 101 | 1 | 0.99 | 5405 | 7 | 0.13 |
| Abs (10 ng/mL) | | | | | | | |
| anti-IFNw auto- | 5.00 | 85 | 0 | 0.00 | 6419 | 6 | 0.09 |
| Abs (10 mg/L) | | | | | | | |
| anti-IFNw auto- | 7.62 | 25 | 1 | 4.00 | 6831 | 3 | 0.04 |
| Abs (10 ng/mL) | | | | | | | |
| anti-IFNw auto- | 11.21 | 3 | 0 | 0.00 | 3493 | 24 | 0.69 |
| Abs (10 ng/mL) | | | | | | | |
| anti-IFNw auto- | 7.84 | 5 | 0 | 0.00 | 2346 | 44 | 1.88 |
| Abs (10 mg/mL) | | | | | | | |
| anti-IFNw auto- | 16.55 | 614 | 5 | 0.8 | 844 | 12 | 1.42 |
| Abs (100 pg/mL) | | | | | | | |
| anti-IFNw auto- | 18.75 | 272 | 2 | 0.74 | 0 | 0 | 0.00 |
| Abs (100 pg/mL) | | | | | | | |
| anti-IFNw auto- | 14.71 | 158 | 1 | 0.63 | 0 | 0 | 0.00 |
| Abs (100 pg/mL) | | | | | | | |
| anti-IFNw auto- | 11.21 | 137 | 1 | 0.73 | 0 | 0 | 0.00 |
| Abs (100 pg/mL) | | | | | | | |
| anti-IFNw auto- | 14.04 | 31 | 1 | 3.23 | 189 | 0 | 0.00 |
| Abs (100 pg/mL) | | | | | | | |
| anti-IFNw auto- | 20.74 | 3 | 0 | 0.00 | 233 | 2 | 0.86 |
| Abs (100 pg/mL) | | | | | | | |
| anti-IFNw auto- | 17.97 | 5 | 0 | 0.00 | 422 | 10 | 2.37 |
| Abs (100 pg/mL) | | | | | | | |
| only anti-IFNa2 | 4.65 | 343 | 0 | 0.00 | 33574 | 50 | 0.15 |
| auto-Abs (10 | | | | | | | |
| ng/mL) | | | | | | | |
| only anti-IFNa2 | 8.33 | 118 | 0 | 0.00 | 9106 | 6 | 0.07 |
| auto-Abs (10 | | | | | | | |
| ng/mL) | | | | | | | |
| only anti-IFNa2 | 7.89 | 102 | 0 | 0.00 | 5404 | 3 | 0.00 |
| auto-Abs (10 | | | | | | | |
| ng/mL) | | | | | | | |
| only anti-IFNa2 | 2.48 | 85 | 0 | 0.00 | 6417 | 2 | 0.03 |
| auto-Abs (10 | | | | | | | |
| ng/mL) | | | | | | | |
| only anti-IFNa2 | 5.33 | 25 | 0 | 0.00 | 6877 | 5 | 0.07 |
| auto-Abs (10 | | | | | | | |
| ng/mL) | | | | | | | |
| only anti-IFNa2 | 4.27 | 3 | 0 | 0.00 | 3482 | 12 | 0.34 |
| auto-Abs (10 | | | | | | | |
| ng/mL ) | | | | | | | |
| only anti-IFNa2 | 4.72 | 5 | 0 | 0.00 | 2285 | 22 | 0.96 |
| auto-Abs (10 | | | | | | | |
| ng/mL) | | | | | | | |
| only and-IFNa2 | 4.12 | 612 | 0 | 0.00 | 823 | 9 | 1.09 |
| auto-Abs (100 | | | | | | | |
| pg/mL) | | | | | | | |
| only anti-IFNa2 | 0.00 | 273 | 0 | 0.00 | 0 | 0 | 0.00 |
| auto-Abs (100 | | | | | | | |
| pg/mL) | | | | | | | |
| only anti-IFNa2 | 6.06 | 154 | 0 | 0.00 | 0 | 0 | 0.00 |
| auto-Abs (100 | | | | | | | |
| pg/mL) | | | | | | | |
| only anti-IFNa2 | 1.72 | 138 | 0 | 0.00 | 0 | 0 | 0.00 |
| auto-Abs (100 | | | | | | | |
| pg/mL) | | | | | | | |
| only anti-IFNa2 | 5.96 | 31 | 0 | 0.00 | 190 | 1 | 0.53 |
| auto-Abs (100 | | | | | | | |
| pg/mL) | | | | | | | |
| only anti-IFNa2 | 3.21 | 3 | 0 | 0.00 | 232 | 1 | 0.43 |
| auto-Abs (100 | | | | | | | |
| pg/mL) | | | | | | | |
| only anti-IFNa2 | 4.31 | 5 | 0 | 0.00 | 401 | 7 | 1.75 |
| auto-Abs (100 | | | | | | | |
| pg/mL) | | | | | | | |
| only anti-IFNw | 0.93 | 342 | 2 | 0.58 | 33599 | 52 | 0.15 |
| auto-Abs (10 | | | | | | | |
| ng/mL) | | | | | | | |
| only anti-IFNw | 0.00 | 118 | 0 | 0.00 | 9106 | 11 | 0.12 |
| auto-Abs (10 | | | | | | | |
| ng/mL) | | | | | | | |
| only anti-IFNw | 0.00 | 101 | 1 | 0.99 | 5405 | 5 | 0.09 |
| auto-Abs (10 | | | | | | | |
| ng/mL) | | | | | | | |
| only anti-IFNw | 0.00 | 85 | 0 | 0.00 | 6419 | 5 | 0.08 |
| auto-Abs (10 | | | | | | | |
| ng/mL) | | | | | | | |
| only anti-IFNw | 1.79 | 25 | 1 | 4.00 | 6831 | 1 | 0.01 |
| auto-Abs (10 | | | | | | | |
| ng/mL) | | | | | | | |
| only anti-IFNw | 1.40 | 3 | 0 | 1.00 | 3493 | 12 | 0.34 |
| auto-Abs (10 | | | | | | | |
| ng/mL) | | | | | | | |
| only anti-IFNw | 0.39 | 5 | 0 | 0.00 | 2342 | 18 | 0.77 |
| auto-Abs (10 | | | | | | | |
| ng/mL) | | | | | | | |
| only anti-IFNw | 7.19 | 61 | 5 | 0.81 | 842 | 4 | 0.48 |
| auto-Abs (100 | | | | | | | |
| pg/mL) | | | | | | | |
| only anti-IFNw | 6.25 | 27 | 2 | 0.74 | 0 | 0 | 0.00 |
| auto-Abs (100 | | | | | | | |
| pg/mL) | | | | | | | |
| only anti-IFNw | 5.88 | 158 | 1 | 0.63 | 0 | 0 | 0.00 |
| auto-Abs (100 | | | | | | | |
| pg/mL) | | | | | | | |
| only anti-IFNw | 4.31 | 137 | 1 | 0.73 | 0 | 0 | 0.00 |
| auto-Abs (100 | | | | | | | |
| pg/mL) | | | | | | | |
| only anti-IFNw | 8.09 | 31 | 1 | 3.23 | 189 | 0 | 0.00 |
| auto-Abs (100 | | | | | | | |
| pg/mL) | | | | | | | |
| only anti-IFNw | 7.83 | 3 | 0 | 0.00 | 233 | 1 | 0.43 |
| auto-Abs (100 | | | | | | | |
| pg/mL) | | | | | | | |
| only anti-IFNw | 7.42 | 5 | 0 | 0.00 | 420 | 3 | 0.71 |
| auto-Abs (100 | | | | | | | |
| pg/mL) | | | | | | | |
| anti-IFN2 auto- | 10.26 | 328 | 0 | 0.00 | 386 | 7 | 1.81 |
| Abs (100 pg/mL) | | | | | | | |
| anti-IFN2 auto- | 16.67 | 155 | 0 | 0.00 | 0 | 0 | 0.00 |
| Abs (100 pg/mL) | | | | | | | |
| anti-IFN2 auto- | 0.00 | 87 | 0 | 0.00 | 0 | 0 | 0.00 |
| Abs (100 pg/mL) | | | | | | | |
| anti-IFN2 auto- | 3.70 | 68 | 0 | 0.00 | 0 | 0 | 0.00 |
| Abs (100 pg/mL) | | | | | | | |
| anti-IFN2 auto- | 14.63 | 12 | 0 | 0.00 | 111 | 1 | 0.90 |
| Abs (100 pg/mL) | | | | | | | |
| anti-IFN2 auto- | 9.09 | 1 | 0 | 0.00 | 123 | 0 | 0.00 |
| Abs (100 pg/mL) | | | | | | | |
| anti-FN2 auto- | 11.11 | 3 | 0 | 0.00 | 152 | 6 | 3.95 |
| Abs (100 pg/mL) | | | | | | | |
| anti-IFNa2 and | 2.6 | 205 | 0 | 0.00 | 16153 | 14 | 0.09 |
| anti-IFNw auto- | | | | | | | |
| Abs (10 ng/mL) | | | | | | | |
| anti-IFNa2 and | 0.00 | 77 | 0 | 0.00 | 4834 | 1 | 0.02 |
| anti-IFNw auto- | | | | | | | |
| Abs (10 ng/mL) | | | | | | | |
| anti-IFNa2 and | 0.00 | 62 | 0 | 0.00 | 2610 | 1 | 0.04 |
| anti-IFNw auto- | | | | | | | |
| Abs (10 ng/mL) | | | | | | | |
| anti-IFNa2 and | 0.00 | 52 | 0 | 0.00 | 2884 | 0 | 0.00 |
| anti-IFNw auto- | | | | | | | |
| Abs (10 ng/mL) | | | | | | | |
| anti-IFNa2 and | 5.00 | 9 | 0 | 0.00 | 2983 | 1 | 0.03 |
| anti-IFNw auto- | | | | | | | |
| Abs (10 ng/mL) | | | | | | | |
| anti-IFNa2 and | 1.96 | 1 | 0 | 0.00 | 1583 | 3 | 0.19 |
| anti-IFNw auto- | | | | | | | |
| Abs (10 mg/mL) | | | | | | | |
| anti-IFNa2 and | 3.00 | 3 | 0 | 0.00 | 1259 | 8 | 0.64 |
| anti-IFNw auto- | | | | | | | |
| Abs (10 ng/mL) | | | | | | | |
| anti-FNa2 and | 6.33 | 332 | 0 | 0.00 | 39 | 2 | 0.51 |
| anti-IFNw auto- | | | | | | | |
| Abs (100 pg/mL) | | | | | | | |
| anti-IFNa2 and | 16.67 | 159 | 0 | 0.00 | 0 | 0 | 0.00 |
| anti-IFNw auto- | | | | | | | |
| Abs (100 pg/mL) | | | | | | | |
| anti-IFNa2 and | 0.00 | 87 | 0 | 0.00 | 0 | 0 | 0.00 |
| anti-IFNw auto- | | | | | | | |
| Abs (100 pg/mL) | | | | | | | |
| anti-IFNa2 and | 0.00 | 68 | 0 | 0.00 | 0 | 0 | 0.00 |
| anti-IFNw auto- | | | | | | | |
| Abs (100 pg/mL] | | | | | | | |
| anti-IFNa2 and | 7.14 | 12 | 0 | 0.00 | 111 | 0 | 0.00 |
| anti-IFNw auto- | | | | | | | |
| Abs (100 pg/mL) | | | | | | | |
| anti-IFNa2 and | 5.45 | 1 | 0 | 0.00 | 124 | 0 | 0.00 |
| anti-IFNw auto- | | | | | | | |
| Abs (100 pg/mL) | | | | | | | |
| anti-IFNa2 and | 8.08 | 3 | 0 | 0.00 | 157 | 2 | 1.27 |
| anti-IFNw auto- | | | | | | | |
| Abs (100 pg/mL) | | | | | | | |
| anti-IFNa2 and/or | 7.96 | 204 | 1 | 0.49 | 15871 | 67 | 0.42 |
| anti-IFNw auto- | | | | | | | |
| Abs (10 ng/mL) | | | | | | | |
| anti-IFNa2 and/or | 0.00 | 77 | 0 | 0.00 | 4830 | 12 | 0.25 |
| anti-IFNw auto- | | | | | | | |
| Abs (10 ng/mL) | | | | | | | |
| anti-IFNa2 and/or | 14.29 | 61 | 1 | 1.64 | 2608 | 8 | 0.31 |
| anti-IFNw auto- | | | | | | | |
| Abs (10 ng/mL) | | | | | | | |
| anti-IFNa2 and/or | 0.00 | 52 | 0 | 0.00 | 2880 | 4 | 0.14 |
| anti-IFNw auto- | | | | | | | |
| Abs (10 ng/mL) | | | | | | | |
| anti-IFNa2 and/or | 12.82 | 9 | 0 | 0.00 | 2940 | 2 | 0.07 |
| anti-IFNw auto- | | | | | | | |
| Abs (10 ng/mL) | | | | | | | |
| anti-IFNa2 and/or | 7.84 | 1 | 0 | 0.00 | 1532 | 12 | 0.78 |
| anti-IFNw auto- | | | | | | | |
| Abs (10 ng/mL) | | | | | | | |
| anti-IFNa2 and/or | 8.08 | 3 | 0 | 0.00 | 1081 | 29 | 2.68 |
| anti-IFNw auto- | | | | | | | |
| Abs (10 ng/mL) | | | | | | | |
| anti-IFNa2 and/or | 19.57 | 321 | 1 | 0.31 | 383 | 8 | 2.09 |
| anti-IFNw auto- | | | | | | | |
| Abs (100 pg/mL) | | | | | | | |
| anti-IFNa2 and/or | 33.33 | 148 | 0 | 0.00 | 0 | 0 | 0.00 |
| anti-IFNw auto- | | | | | | | |
| Abs (100 pg/mL) | | | | | | | |
| anti-IFNa2 and/or | 0.00 | 87 | 1 | 1.15 | 0 | 0 | 0.00 |
| anti-IFNw auto- | | | | | | | |
| Abs (100 pg/mL) | | | | | | | |
| anti-IFNa2 and/or | 12.00 | 68 | 0 | 0.00 | 0 | 0 | 0.00 |
| anti-IFNw auto- | | | | | | | |
| Abs (100 pg/mL) | | | | | | | |
| anti-IFNa2 and/or | 19.51 | 12 | 0 | 0.00 | 109 | 1 | 0.92 |
| anti-IFNw auto- | | | | | | | |
| Ab (100 pg/mL) | | | | | | | |
| anti-IFNa2 and/or | 22.64 | 1 | 0 | 0.00 | 123 | 0 | 0.00 |
| anti-IFNw auto- | | | | | | | |
| Ab (100 pg/mL) | | | | | | | |
| anti-IFNa2 and/or | 20.20 | 3 | 0 | 0.00 | 151 | 7 | 4.64 |
| anti-IFNw auto- | | | | | | | |
| Abs (100 pg/mL) | | | | | | | |
| anti-IFNa2 auto- | 7.89 | 205 | 0 | 0.00 | 16010 | 45 | 0.28 |
| Abs (10 ng/mL) | | | | | | | |
| anti-IFNa2 auto- | 0.00 | 77 | 0 | 0.00 | 4831 | 5 | 0.10 |
| Abs (10 ng/mL) | | | | | | | |
| anti-IFNa2 auto- | 14.29 | 62 | 0 | 0.00 | 2609 | 4 | 0.15 |
| Abs (10 ng/mL) | | | | | | | |
| anti-IFNa2 auto- | 0.00 | 52 | 0 | 0.00 | 2883 | 2 | 0.0 |
| Abs (10 ng/mL) | | | | | | | |
| anti-IFNa2 auto- | 12.50 | 9 | 0 | 1.O | 2976 | 2 | 0.0 |
| Abs (10 ng/mL) | | | | | | | |
| anti-IFNa2 auto- | 7.84 | 1 | 0 | 0.00 | 1553 | 6 | 0.39 |
| Abs (10 ng/mL) | | | | | | | |
| anti-IFNa2 auto- | 8.08 | 3 | 0 | 0.00 | 1158 | 26 | 2.25 |
| Abs (10 ng/mL) | | | | | | | |
| anti-IFNw auto- | 2.63 | 204 | 1 | 0.49 | 16018 | 40 | 0.25 |
| Abs (10 ng/mL) | | | | | | | |
| anti-IFNw auto- | 0.00 | 77 | 0 | 0.00 | 4833 | 8 | 0.17 |
| Abs (10 ng/mL) | | | | | | | |
| anti-IFNw auto- | 0.00 | 61 | 1 | 1.64 | 2609 | 5 | 0.19 |
| Abs (10 ng/mL) | | | | | | | |
| anti-IFNw auto- | 0.00 | 52 | 0 | 0.00 | 2881 | 2 | 0.07 |
| Abs (10 ng/mL) | | | | | | | |
| anti-IFNw auto- | 5.13 | 9 | 0 | 0.00 | 2947 | 1 | 0.03 |
| Abs (10 ng/mL) | | | | | | | |
| anti-IFNw auto- | 1.96 | 1 | 0 | 0.00 | 1562 | 9 | 0.58 |
| Abs (10 ng/mL) | | | | | | | |
| anti-IFNw auto- | 3.00 | 3 | 0 | 0.00 | 1186 | 15 | 1.26 |
| Abs (10 ng/mL) | | | | | | | |
| anti-IFNw auto- | 15.45 | 325 | 1 | 0.31 | 389 | 3 | 0.77 |
| Abs (100 pg/mL) | | | | | | | |
| anti-IFNw auto- | 33.33 | 152 | 0 | 0.00 | 0 | 0 | 0.00 |
| Abs (100 pg/mL) | | | | | | | |
| anti-IFNw auto- | 0.00 | 87 | 1 | 1.15 | 0 | 0 | 0.00 |
| Abs (100 pg/mL) | | | | | | | |
| anti-IFNw auto- | 7.69 | 68 | 0 | 0.00 | 0 | 0 | 0.00 |
| Abs (100 pg/mL) | | | | | | | |
| anti-IFNw auto- | 11.90 | 12 | 0 | 0.00 | 109 | 0 | 0.00 |
| Abs (100 pg/mL) | | | | | | | |
| anti-IFNw auto- | 18.87 | 1 | 0 | 0.00 | 124 | 0 | 0.00 |
| Abs (100 pg/mL) | | | | | | | |
| anti-IFNw auto- | 17.17 | 3 | 0 | 0.00 | 156 | 3 | 1.92 |
| Abs (100 pg/mL) | | | | | | | |
| only anti-IFNa2 | 5.26 | 205 | 0 | 0.00 | 16008 | 29 | 0.18 |
| auto-Abs (10 | | | | | | | |
| ng/mL) | | | | | | | |
| only anti-IFNa2 | 0.00 | 77 | 0 | 0.00 | 4831 | 4 | 0.08 |
| auto-Abs (10 | | | | | | | |
| ng/mL) | | | | | | | |
| only anti-IFNa2 | 14.29 | 62 | 0 | 0.00 | 2609 | 3 | 0.11 |
| auto-Abs (10 | | | | | | | |
| ng/mL) | | | | | | | |
| only anti-IFNa2 | 0.00 | 52 | 0 | 0.00 | 2883 | 2 | 0.07 |
| auto-Abs (10 | | | | | | | |
| ng/mL) | | | | | | | |
| only anti-IFNa2 | 7.50 | 9 | 0 | 0.00 | 2976 | 1 | 0.03 |
| auto-Abs (10 | | | | | | | |
| ng/mL) | | | | | | | |
| only anti-IFNa2 | 5.88 | 1 | 0 | 0.00 | 1553 | 3 | 0.19 |
| auto-Abs (10 | | | | | | | |
| ng/mL) | | | | | | | |
| only anti-IFNa2 | 5.05 | 3 | 0 | 0.00 | 1156 | 16 | 1.38 |
| auto-Abs (10 | | | | | | | |
| ng/mL) | | | | | | | |
| only anti-IFNa2 | 3.85 | 328 | 0 | 0.00 | 386 | 5 | 1.30 |
| auto-Abs (100 | | | | | | | |
| pg/mL) | | | | | | | |
| only anti-IFNa2 | 0.00 | 155 | 0 | 0.00 | 0 | 0 | 0.00 |
| auto-Abs (100 | | | | | | | |
| pg/mL) | | | | | | | |
| only anti-IFNa2 | 0.00 | 87 | 0 | 0.00 | 0 | 0 | 0.00 |
| auto-Abs (100 | | | | | | | |
| pg/mL) | | | | | | | |
| only anti-IFNa2 | 3.70 | 68 | 0 | 0.00 | 0 | 0 | 0.00 |
| auto-Abs (100 | | | | | | | |
| pg/mL) | | | | | | | |
| only anti-IFNa2 | 7.32 | 12 | 0 | 0.00 | 111 | 1 | 0.90 |
| auto-Abs (100 | | | | | | | |
| pg/mL) | | | | | | | |
| only anti-IFNa2 auto-Abs (100 | 3.64 | 1 | 0 | 0.00 | 123 | 0 | 0.00 |
| pg/mL) | | | | | | | |
| only anti-IFNa2 | 3.03 | 3 | 0 | 0.00 | 152 | 4 | 2.63 |
| auto-Abs (100 | | | | | | | |
| pg/mL) | | | | | | | |
| only anti-IFNw | 0.0 | 204 | 1 | 0.49 | 16016 | 24 | 0.15 |
| auto-Abs (10 | | | | | | | |
| ng/mL) | | | | | | | |
| only anti-IFNw | 0.00 | 77 | 0 | 0.00 | 4833 | 7 | 0.14 |
| auto-Abs (10 | | | | | | | |
| ng/mL) | | | | | | | |
| only anti-IFNw | 0.00 | 61 | 1 | 1.64 | 2609 | 4 | 0.15 |
| auto-Abs (10 | | | | | | | |
| ng/mL) | | | | | | | |
| only anti-IFNw | 0.00 | 52 | 0 | 0.00 | 2881 | 2 | 0.07 |
| auto-Abs (10 | | | | | | | |
| ng/mL) | | | | | | | |
| only anti-IFNw | 0.00 | 9 | 0 | 0.00 | 2947 | 0 | 0.00 |
| auto-Abs (10 | | | | | | | |
| ng/mL) | | | | | | | |
| only anti-IFNw | 0.00 | 1 | 0 | 0.00 | 1562 | 6 | 0.38 |
| auto-Abs (10 | | | | | | | |
| ng/mL) | | | | | | | |
| only anti-IFNw | 0.00 | 3 | 0 | 0.00 | 1184 | 5 | 0.42 |
| auto-Abs (10 | | | | | | | |
| ng/mL) | | | | | | | |
| only anti-IFNw | 9.01 | 325 | 1 | 0.31 | 389 | 1 | 0.26 |
| auto-Abs (100 | | | | | | | |
| pg/mL) | | | | | | | |
| only anti-IFNw | 16.67 | 152 | 0 | 0.00 | 0 | 0 | 0.00 |
| auto-Abs (100 | | | | | | | |
| pg/mL) | | | | | | | |
| only anti-IFNw | 0.00 | 87 | 1 | 1.15 | 0 | 0 | 0.00 |
| auto-Abs (100 | | | | | | | |
| pg/mL) | | | | | | | |
| only anti-IFNw | 7.69 | 68 | 0 | 0.00 | 0 | 0 | 0.00 |
| auto-Abs (100 | | | | | | | |
| pg/mL) | | | | | | | |
| only anti-IFNw | 4.76 | 12 | 0 | 0.00 | 109 | 0 | 0.00 |
| auto-Abs (100 | | | | | | | |
| pg/mL) | | | | | | | |
| only anti-IFNw | 13.21 | 1 | 0 | 0.00 | 124 | 0 | 0.00 |
| auto-Abs (100 | | | | | | | |
| pg/mL) | | | | | | | |
| only anti-IFNw | 9.09 | 3 | 0 | 0.00 | 156 | 1 | 0.64 |
| auto-Abs (100 | | | | | | | |
| pg/mL) | | | | | | | |
| anti-IFN2 auto- | 14.84 | 281 | 0 | 0.00 | 437 | 8 | 1.83 |
| Abs (100 pg/mL) | | | | | | | |
| anti-IFN2 auto- | 11.11 | 118 | 0 | 0.00 | 0 | 0 | 0.00 |
| Abs (100 pg/mL) | | | | | | | |
| anti-IFN2 auto- | 17.86 | 67 | 0 | 0.00 | 0 | 0 | 0.00 |
| Abs (100 pg/mL) | | | | | | | |
| anti-IFN2 auto- | 10.11 | 70 | 0 | 0.00 | 0 | 0 | 0.00 |
| Abs (100 pg/mL) | | | | | | | |
| anti-IFN2 auto- | 11.79 | 19 | 0 | 0.00 | 79 | 0 | 0.00 |
| Abs (100 pg/mL) | | | | | | | |
| anti-IFN2 auto- | 18.40 | 2 | 0 | 0.00 | 109 | 2 | 1.83 |
| Abs (100 pg/mL) | | | | | | | |
| anti-IFN2 auto- | 17.31 | 2 | 0 | 0.00 | 249 | 6 | 2.41 |
| Abs (100 pg/mL) | | | | | | | |
| anti-IFNa2 and | 8.31 | 135 | 0 | 0.00 | 17665 | 26 | 0.15 |
| anti-IFNw auto- | | | | | | | |
| Abs (10 ng/mL) | | | | | | | |
| anti-IFNa2 and | 0.00 | 41 | 0 | 0.00 | 4276 | 0 | 0.00 |
| anti-IFNw auto- | | | | | | | |
| Abs (10 ng/mL) | | | | | | | |
| anti-IFNa2 and | 0.00 | 40 | 0 | 0.00 | 2796 | 1 | 0.04 |
| anti-IFNw auto- | | | | | | | |
| Abs (10 ng/mL) | | | | | | | |
| anti-IFNa2 and | 6.38 | 33 | 0 | 0.00 | 3538 | 1 | 0.03 |
| anti-IFNw auto- | | | | | | | |
| Abs (10 ng/mL) | | | | | | | |
| anti-IFNa2 and | 5.91 | 16 | 0 | 0.00 | 3906 | 1 | 0.03 |
| anti-IFNw auto- | | | | | | | |
| Abs (10 ng/mL) | | | | | | | |
| anti-IFNa2 and | 12.27 | 2 | 0 | 0.00 | 1959 | 9 | 0.46 |
| anti-IFNw auto- | | | | | | | |
| Abs (10 ng/mL) | | | | | | | |
| anti-IFNa2 and | 10.26 | 2 | 0 | 0.00 | 1190 | 14 | 1.18 |
| anti-IFNw auto- | | | | | | | |
| Abs (10 ng/mL) | | | | | | | |
| anti-IFNa2 and | 10.34 | 303 | 0 | 0.00 | 457 | 4 | 0.88 |
| anti-IFNw auto- | | | | | | | |
| Abs (100 pg/mL) | | | | | | | |
| anti-IFNa2 and | 10.00 | 134 | 0 | 0.00 | 0 | 0 | 0.00 |
| anti-IFNw auto- | | | | | | | |
| Abs (100 pg/mL) | | | | | | | |
| anti-IFNa2 and | 10.34 | 72 | 0 | 0.00 | 0 | 0 | 0.00 |
| anti-IFNw auto- | | | | | | | |
| Abs (100 pg/mL) | | | | | | | |
| anti-IFNa2 and | 8.89 | 71 | 0 | 0.00 | 0 | 0 | 0.00 |
| anti-IFNw auto- | | | | | | | |
| Abs (100 pg/mL) | | | | | | | |
| anti-IFNa2 and | 5.61 | 19 | 0 | 0.00 | 81 | 0 | 0.00 |
| anti-IFNw auto- | | | | | | | |
| Abs (100 pg/mL) | | | | | | | |
| anti-IFNa2 and | 15.15 | 2 | 0 | 0.00 | 110 | 1 | 0.91 |
| anti-IFNw auto- | | | | | | | |
| Abs (100 pg/mL) | | | | | | | |
| anti-IFNa2 and | 12.03 | 2 | 0 | 0.00 | 266 | 3 | 1.13 |
| anti-IFNw auto- | | | | | | | |
| Abs (100 pg/mL) | | | | | | | |
| anti-IFNa2 and/or | 14.15 | 135 | 1 | 0.74 | 17478 | 75 | 0.43 |
| anti-IFNw auto- | | | | | | | |
| Abs (10 ng/mL) | | | | | | | |
| anti-IFNa2 and/or | 12.50 | 41 | 0 | 0.00 | 4272 | 6 | 0.14 |
| anti-IFNw auto- | | | | | | | |
| Abs (10 ng/mL) | | | | | | | |
| anti-IFNa2 and/or | 6.67 | 40 | 0 | 0.00 | 2795 | 2 | 0.07 |
| anti-IFNw auto- | | | | | | | |
| Abs (10 ng/mL) | | | | | | | |
| anti-IFNa2 and/or | 9.57 | 33 | 0 | 0.00 | 3534 | 4 | 0.1 |
| anti-IFNw auto- | | | | | | | |
| Abs (10 ng/mL) | | | | | | | |
| anti-IFNa2 and/or | 13.11 | 16 | 1 | 6.25 | 3879 | 6 | 0.15 |
| anti-IFNw auto- | | | | | | | |
| Abs (10 ng/mL) | | | | | | | |
| anti-IFNa2 and/or | 18.13 | 2 | 0 | 0.00 | 1901 | 24 | 1.26 |
| anti-IFNw auto- | | | | | | | |
| Abs (10 ng/mL) | | | | | | | |
| anti-IFNa2 and/or | 15.58 | 2 | 0 | 0.00 | 1097 | 33 | 3.01 |
| anti-IFNw auto- | | | | | | | |
| Abs (10 mg/mL) | | | | | | | |
| anti-IFNa2 and/or | 21.57 | 264 | 4 | 1.52 | 433 | 11 | 2.54 |
| anti-IFNw auto- | | | | | | | |
| Abs (100 pg/mL) | | | | | | | |
| anti-IFNa2 and/or | 11.11 | 104 | 2 | 1.92 | 0 | 0 | 0.00 |
| anti-IFNw auto- | | | | | | | |
| Abs (100 pg/mL) | | | | | | | |
| anti-IFNa2 and/or | 25.00 | 66 | 0 | 0.00 | 0 | 0 | 0.00 |
| anti-IFNw auto- | | | | | | | |
| Abs (100 pg/mL) | | | | | | | |
| anti-IFNa2 and/or | 13.48 | 68 | 1 | 1.47 | 0 | 0 | 0.00 |
| anti-IFNw auto- | | | | | | | |
| Abs (100 pg/mL) | | | | | | | |
| anti-IFNa2 and/or | 20.83 | 19 | 1 | 5.26 | 78 | 0 | 0.00 |
| anti-IFNw auto- | | | | | | | |
| Abs (100 pg/mL) | | | | | | | |
| anti-IFNa2 and/or | 24.69 | 2 | 0 | 0.00 | 108 | 3 | 2.78 |
| anti-IFNw auto- | | | | | | | |
| Abs (100 pg/mL) | | | | | | | |
| anti-IFNa2 and/or | 23.87 | 2 | 0 | 0.00 | 247 | 8 | 3.24 |
| anti-IFNw auto- | | | | | | | |
| Abs (100 pg/mL) | | | | | | | |
| anti-IFNa2 auto- | 12.80 | 135 | 0 | 0.00 | 1756 | 50 | 0.28 |
| Abs (10 ng/mL) | | | | | | | |
| anti-IFNa2 auto- | 12.50 | 41 | 0 | 0.00 | 4275 | 2 | 0.05 |
| Abs (10 ng/mL) | | | | | | | |
| anti-IFNa2 auto- | 6.45 | 40 | 0 | 0.00 | 2795 | 1 | 0.04 |
| Abs (10 ng/mL) | | | | | | | |
| anti-IFNa2 auto- | 9.57 | 33 | 0 | 0.00 | 3534 | 1 | 0.03 |
| Abs (10 ng/mL) | | | | | | | |
| anti-IFNa2 auto- | 10.81 | 16 | 0 | 0.00 | 3902 | 6 | 0.15 |
| Abs (10 ng/mL) | | | | | | | |
| anti-IFNa2 auto- | 16.25 | 2 | 0 | 0.00 | 1930 | 19 | 0.98 |
| Abs (10 ng/mL) | | | | | | | |
| anti-IFNa2 auto- | 14.84 | 2 | 0 | 0.00 | 1130 | 21 | 1.86 |
| Abs (10 ng/mL) | | | | | | | |
| anti-IFNw auto- | 9.62 | 135 | 1 | 0.74 | 17582 | 56 | 0.32 |
| Abs (10 ng/mL) | | | | | | | |
| anti-IFNw auto- | 0.00 | 41 | 0 | 0.00 | 4273 | 1 | 0.09 |
| Abs (10 ng/mL) | | | | | | | |
| anti-IFNw auto- | 0.00 | 40 | 0 | 0.00 | 2796 | 2 | 0.07 |
| Abs (10 ng/mL) | | | | | | | |
| anti-IFNw auto- | 6.38 | 33 | 0 | 0.00 | 3538 | 4 | 0.11 |
| Abs (10 ng/mL) | | | | | | | |
| anti-IFNw auto- | 8.15 | 16 | 1 | 6.25 | 3884 | 2 | 0.05 |
| Abs (10 ng/mL) | | | | | | | |
| anti-IFNw auto- | 14.11 | 2 | 0 | 0.00 | 1931 | 15 | 0.78 |
| Abs (10 ng/mL) | | | | | | | |
| anti-IFNw auto- | 10.97 | 2 | 0 | 0.00 | 1160 | 29 | 2.50 |
| Abs (10 ng/mL) | | | | | | | |
| anti-IFNw auto- | 16.95 | 286 | 4 | 1.40 | 455 | 9 | 1.98 |
| Abs (100 pg/mL) | | | | | | | |
| anti-IFNw auto- | 10.00 | 120 | 2 | 1.67 | 0 | 0 | 0.00 |
| Abs (100 pg/mL) | | | | | | | |
| anti-IFNw auto- | 17.24 | 71 | 0 | 0.00 | 0 | 0 | 0.00 |
| Abs (100 pg/mL) | | | | | | | |
| anti-IFNw auto- | 12.22 | 69 | 1 | 1.45 | 0 | 0 | 0.00 |
| Abs (100 pg/mL) | | | | | | | |
| anti-IFNw auto- | 14.51 | 19 | 1 | 5.26 | 80 | 0 | 0.00 |
| Abs (100 pg/mL) | | | | | | | |
| anti-IFNw auto- | 21.34 | 2 | 0 | 0.00 | 109 | 2 | 1.83 |
| Abs (100 pg/mL) | | | | | | | |
| anti-IFNw auto- | 18.47 | 2 | 0 | 0.00 | 266 | 7 | 2.63 |
| Abs (100 pg/mL) | | | | | | | |
| only anti-IFNa2 | 4.42 | 135 | 0 | 0.00 | 17563 | 21 | 0.12 |
| auto-Abs (10 | | | | | | | |
| ng/mL) | | | | | | | |
| only anti-IFNa2 | 12.50 | 41 | 0 | 0.00 | 4275 | 2 | 0.05 |
| auto-Abs (10 | | | | | | | |
| ng/mL) | | | | | | | |
| only anti-IFNa2 | 6.45 | 40 | 0 | 0.00 | 2795 | 0 | 0.00 |
| auto-Abs (10 | | | | | | | |
| ng/mL) | | | | | | | |
| only anti-IFNa2 | 3.19 | 33 | 0 | 0.0 | 3534 | 0 | 0.00 |
| auto-Abs (10 | | | | | | | |
| ng/mL) | | | | | | | |
| only anti-IFNa2 | 4.86 | 16 | 0 | 0.00 | 3901 | 4 | 0.10 |
| auto-Abs (10 | | | | | | | |
| ng/mL) | | | | | | | |
| only anti-IFNa2 | 3.75 | 2 | 0 | 0.00 | 1929 | 9 | 0.47 |
| auto-Abs (10 | | | | | | | |
| ng/mL) | | | | | | | |
| only anti-IFNa2 | 4.52 | 2 | 0 | 0.00 | 1129 | 6 | 0.53 |
| auto-Abs (10 | | | | | | | |
| ng/mL) | | | | | | | |
| only anti-IFNa2 | 4.23 | 281 | 0 | 0.00 | 437 | 4 | 0.92 |
| auto-Abs (100 | | | | | | | |
| pg/mL) | | | | | | | |
| only anti-IFNa2 | 0.00 | 18 | 0 | 0.00 | 0 | 0 | 0.00 |
| auto-Abs (100 | | | | | | | |
| pg/mL) | | | | | | | |
| only anti-IFNa2 | 7.14 | 67 | 0 | 0.00 | 0 | 0 | 0.00 |
| auto-Abs (100 | | | | | | | |
| pg/mL) | | | | | | | |
| only anti-IFNa2 | 1.12 | 70 | 0 | 0.00 | 0 | 0 | 0.00 |
| auto-Abs (100 | | | | | | | |
| pg/mL) | | | | | | | |
| only anti-IFNa2 | 5.67 | 19 | 0 | 0.00 | 79 | 0 | 0.00 |
| auto-Abs (100 | | | | | | | |
| pg/mL) | | | | | | | |
| only anti-IFNa2 | 3.07 | 2 | 0 | 0.00 | 109 | 1 | 0.92 |
| auto-Abs (100 | | | | | | | |
| pg/mL) | | | | | | | |
| only anti-IFNa2 | 5.13 | 2 | 0 | 0.00 | 249 | 3 | 1.20 |
| auto-Abs (100 | | | | | | | |
| pg/mL) | | | | | | | |
| only anti-IFNw | 1.26 | 135 | 1 | 0.74 | 17580 | 28 | 0.16 |
| auto-Abs (10 | | | | | | | |
| ng/mL) | | | | | | | |
| only anti-IFNw | 0.00 | 41 | 0 | 0.00 | 4273 | 4 | 0.09 |
| auto-Abs (10 | | | | | | | |
| ng/mL) | | | | | | | |
| only anti-IFNw | 0.00 | 40 | 0 | 0.00 | 2796 | 1 | 0.04 |
| auto-Abs (10 | | | | | | | |
| ng/mL) | | | | | | | |
| only anti-IFNw | 0.00 | 33 | 0 | 0.0 | 3538 | 3 | 0.08 |
| auto-Abs (10 | | | | | | | |
| ng/mL) | | | | | | | |
| only anti-IFNw | 2.17 | 16 | 1 | 6.25 | 3884 | 1 | 0.03 |
| auto-Abs (10 | | | | | | | |
| ng/mL) | | | | | | | |
| only anti-IFNw | 1.84 | 2 | 0 | 0.00 | 1931 | 6 | 0.31 |
| auto-Abs (10 | | | | | | | |
| ng/mL) | | | | | | | |
| only anti-IFNw | 0.65 | 2 | 0 | 0.00 | 1158 | 13 | 1.12 |
| auto-Abs (10 | | | | | | | |
| ng/mL) | | | | | | | |
| only anti-IFNw | 6.53 | 286 | 4 | 1.40 | 453 | 3 | 0.66 |
| auto-Abs (100 | | | | | | | |
| pg/mL) | | | | | | | |
| only anti-IFNw | 0.00 | 120 | 2 | 1.67 | 0 | 0 | 0.00 |
| auto-Abs (100 | | | | | | | |
| pg/mL) | | | | | | | |
| only anti-IFNw | 6.90 | 71 | 0 | 0.00 | 0 | 0 | 0.00 |
| auto-Abs (100 | | | | | | | |
| pg/mL) | | | | | | | |
| only anti-IFNw | 3.33 | 69 | 1 | 1.45 | 0 | 0 | 0.00 |
| auto-Abs (100 | | | | | | | |
| pg/mL) | | | | | | | |
| only anti-IFNw | 8.81 | 19 | 1 | 5.26 | 80 | 0 | 0.00 |
| auto-Abs (100 | | | | | | | |
| pg/mL) | | | | | | | |
| only anti-IFNw | 6.10 | 2 | 0 | 0.00 | 109 | 1 | 0.92 |
| auto-Abs (100 | | | | | | | |
| pg/mL) | | | | | | | |
| only anti-IFNw | 6.37 | 2 | 0 | 0.00 | 264 | 2 | 0.76 |
| auto-Abs (100 | | | | | | | |
| pg/mL) | | | | | | | |

The prevalence of auto-Abs capable of neutralizing 10 ng/mL IFN-α2 and/or IFN-ω was 1% in individuals between 70 and 80 years of age, and 2.6% in individuals over the age of 80 years. This trend was even more striking in men, with 1.3% positive between the ages of 70 and 80 years, and more than 3% after 80 years. These findings were replicated independently in two cohorts of 704 and 376 elderly individuals from Estonia and Japan, with LIPS and ELISA assays, respectively (FIG. 26F, G). Finally, we tested a random subsample of these elderly individuals for auto-Abs capable of neutralizing 100 pg/mL type I IFN and, again, the prevalence of neutralizing auto-Abs was even higher (FIG. 21G-H, S5H-M), and more than doubling with each decade of age. Indeed, 0.53% of individuals between the ages of 60 and 70 years were positive, 1.3% of those between 70 and 80 years, and 3.8% of those over 80 years. Interestingly, there was no significant difference between men and women (Fisher exact test, P=0.8). Overall, these findings indicate that there is an exponential increase in the prevalence of auto-Abs neutralizing type I IFNs with age in elderly individuals.

Concluding Remarks

We report that at least 20% of patients over 80 years of age with life-threatening COVID-19 pneumonia carry circulating auto-Abs neutralizing 100 pg/mL IFN-α2 and/or IFN-ω, and that such antibodies are present in more than 15% of patients of all ages with this condition. Some of these auto-Abs are hidden and only detectable by a neutralization assay. In addition, at least 20% of deceased individuals in most age groups were found to have such auto-Abs. Importantly, auto-Abs capable of neutralizing high or low concentrations of type I IFNs have been found in patients without inborn errors of TLR3- or TLR7-dependent type I IFN immunity (5) (Asano et al, accompanying report submitted), suggesting that both inborn errors and auto-Abs are independently causal of critical disease. It is also striking that inborn errors are more common in patients below the age of 70 years, whereas auto-Abs are more common in patients over the age of 70 years. We also report that the prevalence of auto-Abs against type I IFNs increases significantly with age in the general population, reaching 0.1%, 1%, and 2.9% in individuals older than 60, 70, and 80 years of age, respectively, for antibodies neutralizing 10 ng/mL type I IFNs, and 0.5%, 1.3%, and 3.8%, respectively, for antibodies neutralizing 100 pg/mL IFN. These auto-Abs provide an explanation for the major increase in the risk of critical COVID-19 in the elderly. This increase with age is consistent with studies of various auto-Abs since the 1960s (30-34). These auto-Abs appear to have remained clinically silent in these individuals until SARS-CoV-2 infection. It is tempting to speculate that an even greater proportion of life-threatening COVID-19 cases are due to auto-Abs neutralizing lower, physiological concentrations of type I IFNs. In vitro, concentrations of type I IFN as low as 100 pg/mL can impair SARS-CoV-2 replication in epithelial cells (FIG. 2A). Moreover, the levels of type I IFN detected in the blood and respiratory tract of patients with acute and benign SARS-CoV-2 infections are in the range of 1 to 100 pg/mL (25, 26). We have shown that antibodies neutralizing 100 pg/mL type I IFN in plasma diluted 1/10, corresponding to the neutralization of 1 ng/mL IFN in vivo, can account for at least 20% of deaths across all ages and of critical cases in the elderly >80 years. We speculate that auto-Abs neutralizing concentrations of type I IFN 10 or 100 times lower than this may be even more common in the general population, and perhaps also pathogenic upon infection with SARS-CoV-2.

Our findings have immediate clinical applications. First, it is easy and rapid to test for auto-Abs against type I IFNs in patients infected with SARS-CoV-2. Screening for these antibodies is even possible in the general population prior to infection. The type I IFN-neutralizing activity of these antibodies is a better read-out than their mere detection, which can be falsely negative. Particular attention should be paid to elderly individuals, and patients with known auto-immune or genetic conditions associated with auto-Abs against type I IFNs (13-18, 21-23). Second, patients with auto-Abs against type I IFN should be vaccinated against COVID-19 as a priority. Third, live-attenuated vaccines, including YFV-17D and vaccines using the YFV-17D backbone against SARS-CoV-2, should not be given to patients with auto-Abs (24, 35). Fourth, these patients appeared to be healthy before SARS-CoV-2 infection, but they should also be carefully followed for other viral illnesses, as exemplified by the adverse reactions to YFV-17D (24). Fifth, in cases of SARS-CoV-2 infection in unvaccinated individuals with auto-Abs against type I IFNs, the patients should be hospitalized for prompt management. Early treatment with monoclonal antibodies (36, 37) can be administered to patients who do not have symptoms of severe COVID-19 pneumonia, and IFN-β can be administered in the absence of both pneumonia and auto-Abs against IFN-β (28, 29). Rescue treatment by plasma exchange is another therapeutic option in patients that already have pneumonia (38). Sixth, blood products should be screened for anti-IFN auto-Abs and any products containing such antibodies should be excluded from donation (10). Plasma from donors convalescing from COVID-19 should be tested for such auto-Abs (10). Seventh, given the documented innocuity and potential efficacy of a single injection, early therapy with IFN-β may be considered for the contacts of contagious subjects or during the first week after infection, even in the absence of, or before the documentation of auto-Abs against type I IFNs, in elderly patients, who have a higher risk of auto-Abs against type I IFN and, therefore, of critical COVID-19 pneumonia (39). Finally, it will be important to decipher the mechanism underlying the development of these auto-Abs, which may be genetic (germline or somatic), epigenetic and/or involve thymic dysfunction. Overall, our findings show that auto-Abs neutralizing concentrations of type I IFN lower than previously reported, but still higher than physiological concentrations, are common in the elderly population. They underlie at least 20% of cases of critical COVID-19 pneumonia in patients over the age of 80 years, at least 20% of COVID-19 deaths at all ages, and their prevalence increases with age in the uninfected general population, reaching at least 3% of individuals after the age of 70 years.

Materials and Methods

Subjects and Samples

We enrolled 2,248 patients with proven life-threatening (critical) COVID-19, 588 asymptomatic or paucisymptomatic individuals with proven COVID-19, and 33,352 healthy controls in this study. All subjects were recruited according to protocols approved by local institutional review boards (IRBs). For patients enrolled in the Italian cohort, ethics approval was obtained from the University of Milano-Bicocca School of Medicine, San Gerardo Hospital, Monza—Ethics Committee of the National Institute of Infectious Diseases Lazzaro Spallanzani (84/2020) (Italy), the IRB of Fondazione IRCCS Policlinico San Matteo, Pavia (Italy), and the Comitato Etico Provinciale (NP 4000—Studio CORONAlab). STORM-Health care workers were enrolled in the STudio OsseRvazionale sullo screening dei lavoratori ospedalieri per COVID-19 (STORM-HCW) study, which was approved by the local IRB on Jun. 18, 2020. For the COV-Contact study, ethics approval was obtained from the CPP IDF VI (ID RCB: 2020-A00280-39). For patients were enrolled in the French COVID cohort (clinicaltrials.gov NCT04262921), ethics approval was obtained from the CPP IDF VI (ID RCB: 2020-A00256-33). Some contact subjects were enrolled in the Cov-Contact cohort (clinicaltrials.gov NCT04259892). Anonymized samples were studied at the NIAID under non-human subject research conditions; no additional IRB consent was required at the NIH. Protocols in San Raffaele were approved by San Raffaele Hospital Ethical Committee. Samples were obtained from the Milieur Intérieur Cohort, which was approved by the Comité de Protection des Personnes—Ouest 6 (Committee for the protection of persons) on Jun. 13, 2012 and by the French Agence nationale de sécurité du médicament (ANSM) on Jun. 22, 2012. The Milieur Intérieur Cohort study is sponsored by Institut Pasteur (Pasteur ID-RCB Number: 2012-A00238-35), and was conducted as a single-center interventional study without an investigational product. The original protocol was registered under ClinicalTrials.gov (study #NCT01699893). The samples and data used in this study were formally established as the Milieu Interieur biocollection (NCT03905993), with approval from the Comit6 de Protection des Personnes -Sud Méditerranée and the Commission nationale de l'informatique et des libertés (CNIL), obtained on Apr. 11, 2018. The content of the manuscript is the sole responsibility of the authors and does not necessarily represent the official views of any of the funding sources. The patients of the Amsterdam UMC Covid-19 cohort were enrolled prospectively at the Amsterdam UMC. Ethics approval was obtained from the Amsterdam UMC Biobank Committee (202_065 #A202029). All protocols conformed to local ethics recommendations and informed consent was obtained when required.

The severity of COVID-19 was assessed for each patient as follows (5, 6). “Life-threatening COVID-19 pneumonia” was defined as pneumonia developing in patients with critical disease, whether pulmonary, with mechanical ventilation (CPAP, BIPAP, intubation, high-flow oxygen), septic shock, or with damage to any other organ requiring admission to the ICU. The controls were individuals infected with SARS-CoV-2 (as demonstrated by a positive PCR and/or serological test and/or displaying typical symptoms, such as anosmia/ageusia after exposure to a confirmed COVID-19 case) who remained asymptomatic or developed mild, self-healing, ambulatory disease with no evidence of pneumonia.

Detection of Anti-Cytokine Autoantibodies

Gyros

Cytokines, recombinant human (rh)IFN-α2 (Milteny Biotec, ref. number 130-108-984) or rhIFN-ω (Merck, ref. number SRP3061), were first biotinylated with EZ-Link Sulfo-NHS-LC-Biotin (Thermo Fisher Scientific, cat. number A39257), according to the manufacturer's instructions, with a biotin-to-protein molar ratio of 1:12. The detection reagent contained a secondary antibody (Alexa Fluor 647 goat anti-human IgG (Thermo Fisher Scientific, ref. number A21445) diluted in Rexip F (Gyros Protein Technologies, ref. number P0004825; 1/500 dilution of the 2 mg/mL stock to yield a final concentration of 4 pg/mL). Buffer PBS-T 0.01% and Gyros Wash buffer (Gyros Protein Technologies, ref. number P0020087) were prepared according to the manufacturer's instructions. Plasma or serum samples were then diluted 1/100 in PBS-T 0.01% and tested with the Bioaffy 1000 CD (Gyros Protein Technologies, ref. number P0004253), and the Gyrolab X-Pand (Gyros Protein Technologies, ref. number P0020520). Cleaning cycles were performed in 20% ethanol.

Multiplex Particle-Based Assay

Serum/plasma samples were screened for autoantibodies against IFN-α2 and IFN-ω in a multiplex particle-based assay, in which magnetic beads with differential fluorescence were covalently coupled to recombinant human proteins (2.5 μg/reaction). Beads were combined and incubated with 1/100-diluted serum/plasma samples for 30 minutes. Each sample was tested once. The beads were then washed and incubated with PE-labeled goat anti-human IgG (1 μg/mL) for an additional 30 minutes. They were then washed again and used for a multiplex assay on a BioPlex X200 instrument.

Enzyme-Linked Immunosorbent Assays (ELISA)

ELISA was performed as previously described (6). In brief, 96-well ELISA plates (MaxiSorp; Thermo Fisher Scientific) were coated by incubation overnight at 4° C. with 2 μg/mL rhIFN-α2 (Milteny Biotec, ref. number 130-108-984), and rhIFN-ω (Merck, ref. number SRP3061). Plates were then washed (PBS 0.005% Tween), blocked by incubation with 5% nonfat milk powder in the same buffer, washed, and incubated with 1:50 dilutions of plasma from the patients or controls for 2 h at room temperature (or with specific mAbs as positive controls). Each sample was tested once. Plates were thoroughly washed. Horseradish peroxidase (HRP)-conjugated Fc-specific IgG fractions from polyclonal goat antiserum against human IgG, IgM or IgA (Nordic Immunological Laboratories) were added to a final concentration of 2 μg/mL. Plates were incubated for 1 h at room temperature and washed. Substrate was added and the optical density (OD) was measured. A similar protocol was used to test for antibodies against 12 subtypes of IFN-α, except that the plates were coated with cytokines from PBL Assay Science (catalog #11002-1), or IFN-β (Milteny Biotech, ref. number: 130-107-888).

LIPS

Levels of autoantibodies against IFN-α subtypes were measured in luciferase-based immunoprecipitation assay (LIPS), as previously described (15). IFNA1, IFNA2, IFNA8, and IFNA21 sequences were inserted into a modified pPK-CMV-F4 fusion vector (PromoCell GmbH, Germany), in which the firefly luciferase replaced the NanoLuc luciferase (Promega, USA). The resulting constructs were used to transfect HEK293 cells and the IFNA-luciferase fusion proteins were collected in the tissue culture supernatant. For autoantibody screening, we combined 2×10⁶ luminescence units (LU) of IFNA1, IFNA2, IFNA8 and IFNA21 in a single IP reaction mixture (pool 1), and IFNA4, IFNA5, IFNA6 and IFNA7 in another IP reaction mixture (pool 2). Serum samples were incubated with Protein G agarose beads (Exalpha Biologicals, USA) at room temperature for 1 h in a 96-well microfilter plate (Merck Millipore, Germany), and we then added 2×10⁶ luminescence units (LU) of antigen and incubated for another hour. Each sample was tested once. The plate was washed with a vacuum system and Nano-Glo® Luciferase Assay Reagent (Promega, USA) was added. Luminescence intensity was measured with a VICTOR X Multilabel Plate Reader (PerkinElmer Life Sciences, USA). The results are expressed in arbitrary units (AU), as a fold-difference relative to the mean of the negative control samples.

Functional Evaluation of Anti-Cytokine Autoantibodies

pSTAT1 Induction in PBMC

The blocking activity of anti-IFN-α2 and anti-IFN-ω auto-Abs was determined by assessing STAT1 phosphorylation in healthy control cells following stimulation with the appropriate cytokines in the presence of 10% healthy control or patient serum/plasma. Surface-stained healthy control PBMCs (350,000/reaction) were cultured in serum-free RPMI medium with 10% healthy control or patient serum/plasma and were either left unstimulated or were stimulated with IFNa or IFNw (10 ng/mL) for 15 minutes at 37° C. Each sample was tested once. Cells were fixed, permeabilized, and stained for intranuclear phospho-STAT1 (Y701). Cells were acquired on a BD LSRFortessa cytometer with gating on CD14⁺ monocytes and the data were analyzed with FlowJo software.

Luciferase Reporter Assays

The blocking activity of anti-IFN-α2 and anti-IFN-ω auto-Abs was determined with a reporter luciferase activity. Briefly, HEK293T cells were transfected with a plasmid containing the firefly luciferase gene under the control of the human ISRE promoter in the pGL4.45 backbone, and a plasmid constitutively expressing Renilla luciferase for normalization (pRL-SV40). Cells were transfected in the presence of the X-tremeGene 9 transfection reagent (Sigma Aldrich, ref. number 6365779001) for 36 hours. Cells in Dulbecco's modified Eagle medium (DMEM, Thermo Fisher Scientific) supplemented with 2% fetal calf serum (FCS) and 10% healthy control or patient serum/plasma were either left unstimulated or were stimulated with IFN-α2 (Milteny Biotec, ref. number 130-108-984), IFN-ω (Merck, ref. number SRP3061), at 10 ng/mL or 100 pg/mL, or IFN-β (Milteny Biotech, ref. number: 130-107-888) at 10 ng/mL, for 16 hours at 37° C. Each sample was tested once for each cytokine and dose. Finally, cell were lysed for 20 minutes at room temperature and luciferase levels were measured with the Dual-Luciferase® Reporter 1000 assay system (Promega, ref. number E1980), according to the manufacturer's protocol. Luminescence intensity was measured with a VICTOR X Multilabel Plate Reader (PerkinElmer Life Sciences, USA). Firefly luciferase activity values were normalized against Renilla luciferase activity values. These values were then normalized against the median induction level for non-neutralizing samples, and expressed as a percentage. Samples were considered to be neutralizing if luciferase induction, normalized against Renilla luciferase activity, was below 15% of the median values for controls tested the same day.

Statistical Analysis

Comparison of proportions of individuals with neutralizing autoantibodies according to categorical variables were performed using a Fisher exact test, as implemented in R (https://cran.r-project.org/). Impact of age in years on the presence of neutralizing autoantibodies was tested by means of logistic regression using the Wald test and adjusting on sex.

Schematic Representation

Schematic representations (FIG. 1B) were created with BioRender.com.

SARS-CoV-2 Experiment

SARS-CoV-2 strain USA-WA1/2020 was obtained from BEI Resources and amplified in Caco-2 cells at 37° C. Viral titers were measured on Huh-7.5 hepatoma cells in a standard plaque assay. Caco-2 (H. sapiens, sex: male, colon epithelial) and Huh-7.5 cells (H. sapiens, sex: male, liver epithelial) were cultured in DMEM supplemented with 1% nonessential amino acids (NEAA) and 10% fetal bovine serum (FBS) at 37° C., under an atmosphere containing 5% CO₂. Both cell lines have been tested negative for contamination with mycoplasma.

SARS-CoV-2 experiments were performed as follows. Huh-7.5 cells were used to seed 96-well plates at a density of 7.5×10³ cells/well. The following day, plasma samples or a commercial anti-IFN-α2 antibody (catalog number 21100-1; R&D Systems) were diluted to 1% and incubated with 5 pM (˜96 pg/mL) or 20 pM (˜384 pg/mL) recombinant IFN-α2 (catalog number 11101-2; R&D systems) for 1 h at 37° C. (dilutions: plasma samples=1/100 and anti-IFN-α2 antibody=1/1,000). Molar ratio was calculated according to the manufacturer's datasheet and with molbiol.ru/eng/scripts/01_04.html. Following this incubation period, the cell culture medium was removed from the 96-well plates by aspiration and replaced with the plasma/anti-IFN-α2 antibody and IFN-α2 mixture. Each sample was tested once, in triplicate. The plates were incubated overnight and the plasma/anti-IFN-α2 antibody plus IFN-α2 mixture was removed by aspiration. The cells were washed once with PBS to remove potential anti-SARS-CoV-2-neutralizing antibodies and fresh medium was then added. Cells were then infected with SARS-CoV-2 by directly adding the virus to the wells. Cells infected at a MOI of 0.05 PFU/cell and incubated at 33° C. for 48 h. The cells were fixed with 7% formaldehyde, stained for SARS-CoV-2 with an anti-N antibody (catalog no. GTX135357; GeneTex), imaged and analyzed as previously described (6).

### REFERENCES

1. A. T. Levin et al., Assessing the age specificity of infection fatality rates for COVID-19: systematic review, meta-analysis, and public policy implications. Eur J Epidemiol 35, 1123-1138 (2020).
2. M. O'Driscoll et al., Age-specific mortality and immunity patterns of SARS-CoV-2. Nature 590, 140-145 (2021).
3. E. J. Williamson et al., Factors associated with COVID-19-related death using OpenSAFELY. Nature 584, 430-436 (2020).
4. J. L. Casanova, H. C. Su, C. H. G. Effort, A Global Effort to Define the Human Genetics of Protective Immunity to SARS-CoV-2 Infection. Cell 181, 1194-1199 (2020).
5. Q. Zhang et al., Inborn errors of type I IFN immunity in patients with life-threatening COVID-19. Science 370, (2020).
6. P. Bastard et al., Autoantibodies against type I IFNs in patients with life-threatening COVID-19. Science 370, (2020).
7. R. Koning et al., Autoantibodies against type I interferons are associated with multi-organ failure in COVID-19 patients. Intensive Care Med, (2021).
8. M. G. P. v. d. Wijst et al., Longitudinal single-cell epitope and RNA-sequencing reveals the immunological impact of type 1 interferon autoantibodies in critical COVID-19. Submitted (2021).
9. J. Troya et al., Neutralizing Autoantibodies to Type I IFNs in >10% of Patients with Severe COVID-19 Pneumonia Hospitalized in Madrid, Spain. J Clin Immunol, (2021).
10. S. E. Vazquez et al., Neutralizing Autoantibodies to Type I Interferons in COVID-19 Convalescent Donor Plasma. J Clin Immunol, (2021).
11. D. Goncalves et al., Antibodies against type-I Interferon: detection and association with severe clinical outcome in COVID-19 patients. medRxiv, (2021).
12. E. Y. Wang et al., Diverse Functional Autoantibodies in Patients with COVID-19. Nature, (2021).
13. M. Levin, Anti-interferon auto-antibodies in autoimmune polyendocrinopathy syndrome type 1. PLoS Med 3, e292 (2006).
14. A. Meager et al., Anti-interferon autoantibodies in autoimmune polyendocrinopathy syndrome type 1. PLoS Med 3, e289 (2006).
15. S. Meyer et al., AIRE-Deficient Patients Harbor Unique High-Affinity Disease-Ameliorating Autoantibodies. Cell 166, 582-595 (2016).
16. P. Bastard et al., Preexisting autoantibodies to type I IFNs underlie critical COVID-19 pneumonia in patients with APS-1. J Exp Med 218, (2021).
17. J. E. Walter et al., Broad-spectrum antibodies against self-antigens and cytokines in RAG deficiency. J Clin Invest 125, 4135-4148 (2015).
18. J. M. Rosenberg et al., Neutralizing Anti-Cytokine Autoantibodies Against Interferon-alpha in Immunodysregulation Polyendocrinopathy Enteropathy X-Linked. Front Immunol 9, 544 (2018).
19. A. Vallbracht, J. Treuner, B. Flehmig, K. E. Joester, D. Niethammer, Interferon-neutralizing antibodies in a patient treated with human fibroblast interferon. Nature 289, 496-497 (1981).
20. R. A. Rudick et al., Incidence and significance of neutralizing antibodies to interferon beta-1a in multiple sclerosis. Multiple Sclerosis Collaborative Research Group (MSCRG). Neurology 50, 1266-1272 (1998).
21. S. Panem, I. J. Check, D. Henriksen, J. Vilcek, Antibodies to alpha-interferon in a patient with systemic lupus erythematosus. J Immunol 129, 1-3 (1982).
22. H. Shiono et al., Spontaneous production of anti-IFN-alpha and anti-IL-12 autoantibodies by thymoma cells from myasthenia gravis patients suggests autoimmunization in the tumor. Int Immunol 15, 903-913 (2003).
23. I. Bello-Rivero et al., Characterization of the immunoreactivity of anti-interferon alpha antibodies in myasthenia gravis patients. Epitope mapping. J Autoimmun 23, 63-73 (2004).
24. P. Bastard et al., Auto-antibodies to type I IFNs can underlie adverse reactions to yellow fever live attenuated vaccine. J Exp Med 218, (2021).
25. J. Hadjadj et al., Impaired type I interferon activity and inflammatory responses in severe COVID-19 patients. Science 369, 718-724 (2020).
26. S. Trouillet-Assant et al., Type I IFN immunoprofiling in COVID-19 patients. JAllergy Clin Immunol 146, 206-208 e202 (2020).
27. N. Honda, U. Lindberg, P. Andersson, S. Hoffmann, H. Takei, Simultaneous multiple immunoassays in a compact disc-shaped microfluidic device based on centrifugal force. Clin Chem 51, 1955-1961 (2005).
28. P. Bastard et al., Interferon-beta Therapy in a Patient with Incontinentia Pigmenti and Autoantibodies against Type I IFNs Infected with SARS-CoV-2. J Clin Immunol, (2021).
29. P. D. Monk et al., Safety and efficacy of inhaled nebulised interferon beta-1a (SNG001) for treatment of SARS-CoV-2 infection: a randomised, double-blind, placebo-controlled, phase 2 trial. Lancet Respir Med 9, 196-206 (2021).
30. B. Hooper, S. Whittingham, J. D. Mathews, I. R. Mackay, D. H. Curnow, Autoimmunity in a rural community. Clin Exp Immunol 12, 79-87 (1972).
31. S. Shu, R. J. Nisengard, W. L. Hale, E. H. Beutner, Incidence and titers of antinuclear, antismooth muscle, and other autoantibodies in blood donors. J Lab Clin Med 86, 259-265 (1975).
32. K. Potocka-Plazak, A. Pituch-Noworolska, J. Kocemba, [Prevalence of autoantibodies in serum of healthy persons over 85 years of age]. Przegl Lek 52, 544-546 (1995).
33. C. G. Parks et al., Reproductive and hormonal risk factors for antinuclear antibodies (ANA) in a representative sample of U.S. women. Cancer Epidemiol Biomarkers Prev 23, 2492-2502 (2014).
34. E. Myasoedova, J. Davis, E. L. Matteson, C. S. Crowson, Is the epidemiology of rheumatoid arthritis changing? Results from a population-based incidence study, 1985-2014. Ann Rheum Dis 79, 440-444 (2020).
35. L. Sanchez-Felipe et al., A single-dose live-attenuated YF17D-vectored SARS-CoV-2 vaccine candidate. Nature 590, 320-325 (2021).
36. P. Chen et al., SARS-CoV-2 Neutralizing Antibody LY-CoV555 in Outpatients with Covid-19. NEngl J Med 384, 229-237 (2021).
37. D. M. Weinreich et al., REGN-COV2, a Neutralizing Antibody Cocktail, in Outpatients with Covid-19. NEngl J Med 384, 238-251 (2021).
38. N. de Prost et al., Plasma Exchange to Rescue Patients with Autoantibodies Against Type I Interferons and Life-Threatening COVID-19 Pneumonia. J Clin Immunol, (2021).
39. D. C. Vinh, L. Abel, P. Bastard, C. J L., I. Meyts, Harnessing type I IFN immunity against SARS-CoV-2 with early administration of IFN-beta. JoCI In Press, (2021).

### Example 5

### Human TLR7 and Plasmacytoid Dendritic Cells are Essential for Type I IFN Pulmonary Immunity to SARS-CoV-2

Autosomal inborn errors of type I IFN immunity and autoantibodies against these cytokines underlie at least 10% of critical COVID-19 pneumonia cases. We report very rare, biochemically deleterious X-linked TLR7 variants in 14 unrelated male individuals aged 13 to 71 years (mean: 36.7 years) from a cohort of 1,005 male individuals aged 0.5 to 99 years (mean: 52.9 years) with unexplained critical COVID-19 pneumonia. None of the 326 asymptomatically infected male individuals aged 1.3 to 102 years (mean: 41.1 years) tested carried such TLR7 variants (p=6×10⁻⁶). The cumulative allele frequency for deleterious TLR7 variants in the male general population was <6.5×10⁻⁴. We also found that blood lymphoid cell lines and myeloid cell subsets from the patients failed to respond to TLR7 stimulation, a phenotype rescued with wild-type TLR7. Finally, the patients' blood plasmacytoid dendritic cells (pDCs) produced low levels of type I IFNs in response to SARS-CoV-2. X-linked recessive TLR7 deficiency is a genetic etiology of life-threatening COVID-19 pneumonia in about 1.6% of male patients below the age of 70 years. Human TLR7 and pDCs are essential for protective type I IFN immunity against SARS-CoV-2 in the lungs.

Introduction

Inter-individual clinical variability in the course of SARS-CoV-2 infection is vast, ranging from silent infection to lethal disease (1). The greatest risk factor for life-threatening COVID-19 pneumonia is age, with a doubling in risk every five years from the age of five years onward, and a sharp rise after the age of 65 years (2, 3). Other epidemiological risk factors, including common genetic variants, have only modest effects, with odds ratios (ORs)<2 and typically <1.5 (2). One intriguing observation is the approximately 1.5-fold higher risk in men, which seems to be age-independent (2-4). COVID Human Genetic Effort (covidhge.com) has enrolled an international cohort of patients, with the aim of investigating genetic and immunological causes of life-threatening COVID-19 pneumonia. We tested the hypothesis that critical influenza and critical COVID-19 can be allelic (5-7), and showed that life-threatening COVID-19 pneumonia can be caused by rare inborn errors of autosomal genes controlling TLR3- and IRF7-dependent type I IFN immunity (8). These disorders were found in 23 men and women aged 17 to 77 years (mean: 48 years). Remarkably, four unrelated patients aged 25 to 50 years had autosomal recessive IFNAR1 (n=2) or IRF7 (n=2) deficiency. These patients had no previous history of severe viral illness, including influenza pneumonia, implying that these genetic disorders unexpectedly show incomplete penetrance for critical influenza. These findings revealed that TLR3- and IRF7-dependent type I IFN immunity is essential for host defense against SARS-CoV-2 infection in the respiratory tract.

We found pre-existing neutralizing auto-Abs against type I IFN in at least 10% of the patients from this same cohort (9). These auto-Abs were found in 101 patients, mostly men (95%), and older than the rest of the cohort, including patients with inborn errors, as they were aged 25 to 87 years (mean: 65 years). These findings have been replicated in five other cohorts (10-15). These auto-Abs predated SARS-CoV-2 infection and were causal for critical COVID-19 pneumonia, because (i) they were found in samples drawn before infection in some patients (9), (ii) they were found in about 0.3% of the general population before the age of 65 years (9), (iii) they were absent from patients with asymptomatic or paucisymptomatic (mild) SARS-CoV-2 infection (9), (iv) they were of childhood onset in patients with various disorders, including autoimmune polyendocrinopathy type I (APS-1), known to be at very high risk of life-threatening COVID-19 (16), and (v) they have been shown to underlie a third of adverse reactions to the live attenuated viral vaccine for yellow fever (17). Collectively, these studies showed that type I IFNs are essential for protective immunity to SARS-CoV-2 in the respiratory tract, but otherwise surprisingly redundant. Auto-Abs against type I IFNs also provide a first explanation for both the biased sex ratio and the higher risk of critical COVID-19 in patients over the age of 65 years. We tested the hypothesis that critical and unexplained COVID-19 pneumonia in younger men may be due to rare variants on the X-chromosome.

Enrichment for Very Rare TLR7 Non-Synonymous Variants in Male Patients

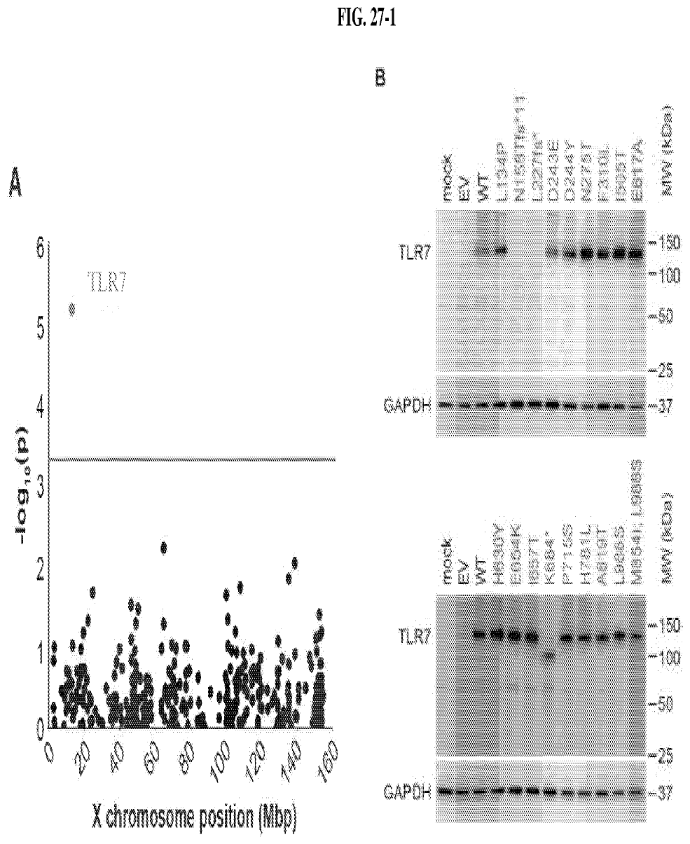

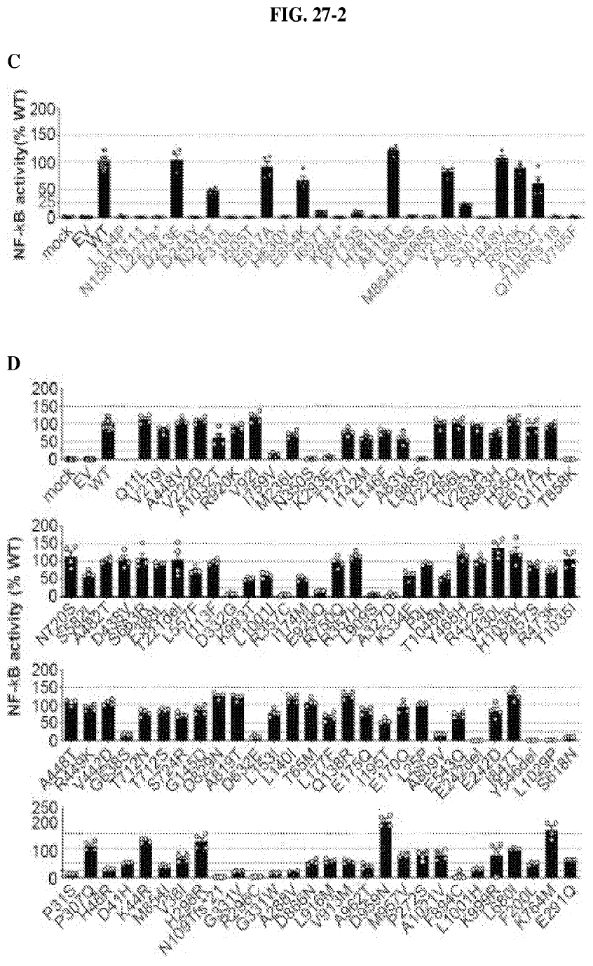

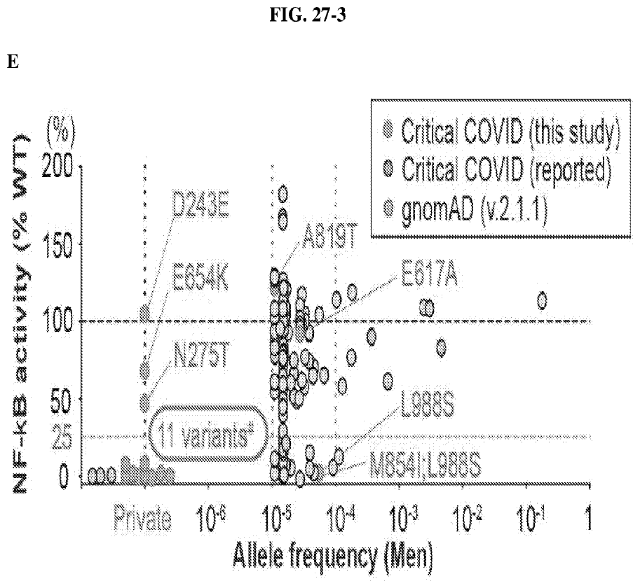

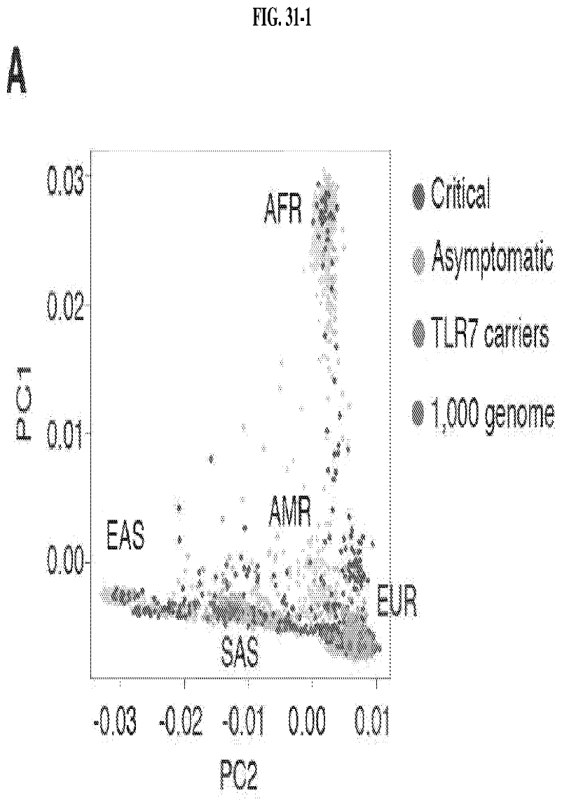

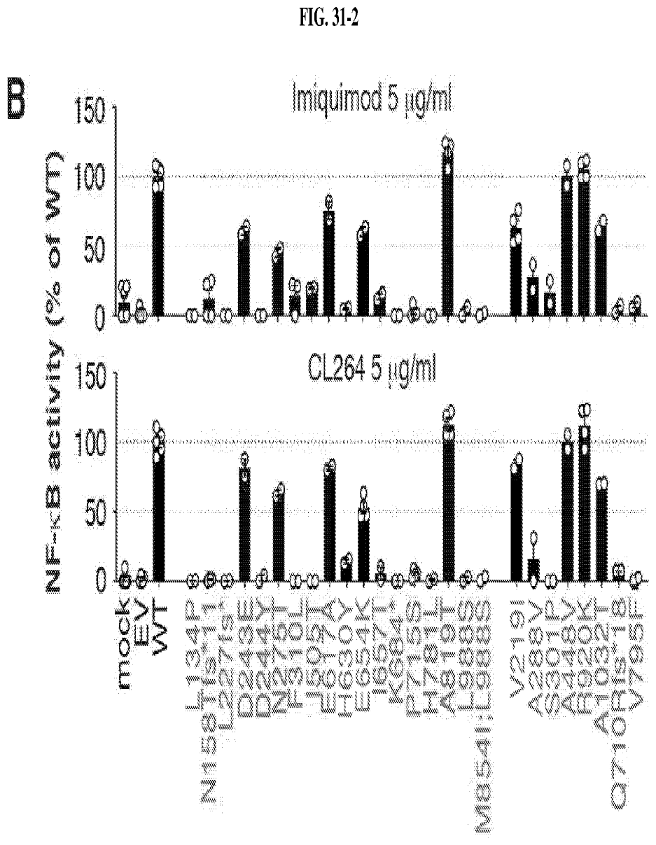

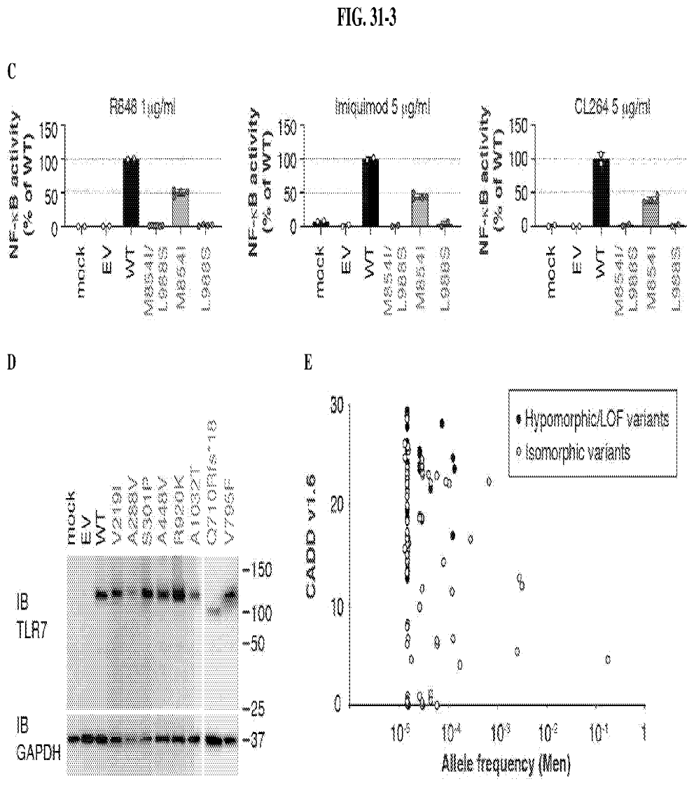

We tested the hypothesis of genetic homogeneity for X-linked recessive disorders in male individuals with life-threatening COVID-19 pneumonia (hereafter referred to as “patients”). We analyzed an international cohort of 1,005 male individuals aged 6 months to 99 years (mean: 5219 years) with no known inborn errors of TL1R3- and IRF7-dependent type I IFN immunity (8) and without neutralizing auto-Abs against type I IFNs (9) (accompanying paper by Bastard P. et al) (Table 12). We also analyzed 326 asymptomatic or paucisymptomatic infected patients aged 1.3 to 102 years (mean: 41.1 years), with positive results for PCR and/or serological screening for SARS-CoV-2 infection (the controls) (Table 12). We sequenced the exomes (n=934) or genomes (n=397) of these patients. We selected in-frame and out-of-frame non-synonymous variants of protein-coding exons that are very rare, that is, with a MAF below 10⁻⁴ in the full gnomAD database (v2.1.1) containing sequences from both male and female individuals. We compared the proportions of patients and controls carrying at least one qualifying variant, by logistic regression adjusted for age and ethnicity (FIG. 31A). We found 190 of 731 genes on the X chromosome with at least five patients carrying non-synonymous variants (Table 13). TLR7 was the highest ranked of these genes (P-value=6×10⁻⁶), with 19 patients carrying one very rare (n=4 patients), two very rare (n=1 patient), or one private (n=14 patients) non-synonymous variants (P-value=6×10⁻⁶) (FIG. 27A—Table 14). One variant (L988S) was recurrent, found in three patients, including a patient carrying two very rare variants (M854I; L988S). No such variants were found in the controls. The same analysis performed on very rare (MAF<10⁻⁴) synonymous TLR7 variants showed no enrichment in patients (one carrier) relative to controls (three carriers). Human TLR7 is an endosomal receptor of ribonucleic acids expressed by B cells and myeloid subsets (18-22), the stimulation of which in plasmacytoid dendritic cells (pDCs) results in the production of large amounts of type I IFN (23-25). We observed no significant enrichment for coding non-synonymous variants of the X-linked gene TLR8 (P-value=0.27, Table 14), the product of which, TLR8, is endosomal and can be stimulated by some synthetic TLR7 agonists, with an expression pattern and signaling pathway overlapping those of TLR7 (26, 27). Unlike TLR7, TLR8 is expressed on granulocytes but not pDCs, possibly accounting for its gain-of-function mutations underlying a phenotype different from type I interferonopathies (28-30). Overall, we found an enrichment in very rare or private non-synonymous TLR7 variants among male patients with critical COVID-19 pneumonia (n=19, 1.9% of our cohort, including only one man over the age of 70 years).

The TLR7 Mutant Alleles of 14 of the 19 Patients are Biochemically Deleterious

The 19 patients carried 18 different TLR7 alleles. We expressed the 18 TLR7 mutant proteins in human embryonic kidney (HEK) 293T cells, which have no endogenous TLR7 and TLR8 expression (31), by transient transfection with the corresponding cDNAs. Immunoblotting of protein extracts with a TLR7-specific mAb showed an absence of TLR7 protein for p.N158Tfs*11 and p.L227fs* and a truncated protein for K684* (FIG. 27B). The other mutant TLR7 proteins were produced in normal amounts (FIG. 27B). We tested their function by cotransfection with an NF-κB-specific reporter. We measured luciferase activity upon stimulation with R848, an agonist of both TLR7 and TLR8 (FIG. 27C). Eleven of the 18 alleles were loss-of-function (LOF) (including L988S in two patients, and M854I; L988S in another), two (p.I657T and p.P715S) were hypomorphic (activity <25%), and the remaining five were neutral (FIG. 27C, Table 15). Similar results were obtained with imiquimod and CL264, two TLR7-specific agonists (FIG. 31B, 31C). We also tested eight other private (p.S301P, p.Q710Rfs*18, p.V795F), very rare (MAF <10⁻⁴; p.A288V) or rare (MAF between 10⁻⁴ and 10^{-hu 2}; p.V2191, p.A448V, p.R920K, p.A1032T) TLR7 variants previously reported in patients with critical COVID-19 but without biochemical characterization (32, 33). These variants were expressed as truncated or full-length proteins (FIG. 31D). The proteins encoded by the three private variants were found to be LOF, that encoded by the very rare variant (p.A288V) was hypomorphic, and those encoded by the four rare variants were neutral (FIG. 27C, FIG. 31B). Collectively, these findings suggest that 14 of the 19 patients in our cohort (Table 11) and six of the previously reported 12 patients carry deleterious TLR7 variants.

TABLE 11
X-linked TLR7 deleterious variants in unrelated male
patients with life-threatening COVID-19 pneumonia

| | | Age | | |
|---|---|---|---|---|
| Patient | Genotype | [years] | Ancestry/residence | Outcome |
| P1 | L134P/Y | 45 | Paraguay/Spain | Survived |
| P2 | N158Tfs11*/Y | 60 | France | Deceased |
| P3 | L227fs*/Y | 34 | Iran | Survived |
| P4 | D244Y/Y | 13 | Turkey | Survived |
| P5 | F310L/Y | 39 | Iran | Survived |
| P6 | I505T/Y | 55 | Italy | Survived |
| P7 | H630Y/Y | 50 | Spain | Survived |
| P8 | I657T/Y | 18 | Italy | Survived |
| P9 | K684*/Y | 30 | Spain | Survived |
| P10 | P715S/Y | 40 | Colombia | Survived |
| P11 | H781L/Y | 13 | Russia/France | Survived |
| P12 | L988S/Y | 26 | Iran | Deceased |
| P13 | L988S/Y | 20 | Turkey | Survived |
| P14 | M854I; L988S/Y | 71 | Italy | Survived |
| P15 | N75H | | | |
| P16 | L372M | | | |
| P17 | F670Lfs*8 | | | |

TABLE 12
Characteristic of the cohort of life-threatening COVID-19 pneumonia and the cohort control of
asymptomatic or paucisymptomatic individuals

| | N | N with age | Age mean | sd | MEDIAN | MIN | MAX |
|---|---|---|---|---|---|---|---|
| Cases | 1005 | 987 | 52.9 | 15.6 | 53.0 | 0.5 | 99 |
| Controls | 326 | 317 | 41.1 | 16.1 | 40.0 | 1.3 | 102 |

TABLE 13
Selection of genes on chromosome X with 5 or more hemizygous carriers.

| | | | | | Ncases_ | Nctrl_ | | | | |
|---|---|---|---|---|---|---|---|---|---|---|
| CHR | Models | model | Ncases | Nctrl | carriers | carriers | N | OR | P-value | P_{Bonferronicorrected} |
| X | hemizygous | TLR7 | 1005 | 326 | 19 | 0 | 19 | 12025656 | 5.96E−06 | 1.13E−03 |
| X | hemizygous | ZC3H12B | 1005 | 326 | 3 | 5 | 8 | 0.063837 | 0.00563332 | 1.00 |
| X | hemizygous | MCF2 | 1005 | 326 | 7 | 0 | 7 | 3285287 | 0.00869198 | 1.00 |
| X | hemizygous | MAP7D3 | 1005 | 326 | 8 | 0 | 8 | 2495162 | 0.01377879 | 1.00 |
| X | hemizygous | IRS4 | 1005 | 326 | 8 | 0 | 8 | 2182535 | 0.01764981 | 1.00 |
| X | hemizygous | POLA1 | 1005 | 326 | 10 | 1 | 11 | 1642866 | 0.02045208 | 1.00 |
| X | hemizygous | SRPX2 | 1005 | 326 | 5 | 0 | 5 | 1458377 | 0.02194455 | 1.00 |
| X | hemizygous | SHROOM4 | 1005 | 326 | 11 | 1 | 12 | 1252448 | 0.0326101 | 1.00 |
| X | hemizygous | ARMCX2 | 1005 | 326 | 5 | 0 | 5 | 1014444 | 0.04412556 | 1.00 |
| X | hemizygous | PHEX | 1005 | 326 | 7 | 0 | 7 | 1826922 | 0.04546455 | 1.00 |
| X | hemizygous | PPPIR3F | 1005 | 326 | 8 | 0 | 8 | 1485836 | 0.04917914 | 1.00 |
| X | hemizygous | G6PD | 1005 | 326 | 3 | 4 | 7 | 0.201194 | 0.06200739 | 1.00 |
| X | hemizygous | SH3KBP1 | 1005 | 326 | 6 | 0 | 6 | 1774796 | 0.06458935 | 1.00 |
| X | hemizygous | SLC9A7 | 1005 | 326 | 6 | 0 | 6 | 1461520 | 0.07771555 | 1.00 |
| X | hemizygous | DGAT2L6 | 1005 | 326 | 6 | 0 | 6 | 552742.6 | 0.0912534 | 1.00 |
| X | hemizygous | ATXN3L | 1005 | 326 | 5 | 0 | 5 | 765016.8 | 0.09265719 | 1.00 |
| X | hemizygous | ATRX | 1005 | 326 | 15 | 1 | 16 | 4.365769 | 0.0955476 | 1.00 |
| X | hemizygous | ZMAT1 | 1005 | 326 | 5 | 0 | 5 | 889670.1 | 0.09558978 | 1.00 |
| X | hemizygous | ZCCHC5 | 1005 | 326 | 8 | 0 | 8 | 997431.4 | 0.09592991 | 1.00 |
| X | hemizygous | ARSF | 1005 | 326 | 9 | 0 | 9 | 1130604 | 0.09773911 | 1.00 |
| X | hemizygous | MTMR8 | 1005 | 326 | 3 | 4 | 7 | 0.243 | 0.10078192 | 1.00 |
| X | hemizygous | PHKA1 | 1005 | 326 | 5 | 0 | 5 | 646383 | 0.10115663 | 1.00 |
| X | hemizygous | BEND2 | 1005 | 326 | 7 | 0 | 7 | 1113882 | 0.10238587 | 1.00 |
| X | hemizygous | BCOR | 1005 | 326 | 7 | 6 | 13 | 0.340619 | 0.10368151 | 1.00 |
| X | hemizygous | LRCH2 | 1005 | 326 | 4 | 3 | 7 | 0.226707 | 0.10497478 | 1.00 |
| X | hemizygous | RGAG1 | 1005 | 326 | 9 | 1 | 10 | 4.36843 | 0.1151885 | 1.00 |
| X | hemizygous | ATP11C | 1005 | 326 | 6 | 4 | 10 | 0.300487 | 0.12007065 | 1.00 |
| X | hemizygous | FMR1 | 1005 | 326 | 2 | 3 | 5 | 0.180243 | 0.12947676 | 1.00 |
| X | hemizygous | ATP1B4 | 1005 | 326 | 4 | 1 | 5 | 5.297248 | 0.1326298 | 1.00 |
| X | hemizygous | GYG2 | 1005 | 326 | 7 | 1 | 8 | 4.754569 7 | 0.14358793 | 1.00 |
| X | hemizygous | DNASEIL1 | 1005 | 326 | 8 | 1 | 9 | 4.034018 | 0.15782359 | 1.00 |
| X | hemizygous | GPR112 | 1005 | 326 | 20 | 9 | 29 | 0.520391 | 0.16023903 | 1.00 |
| X | hemizygous | AFF2 | 1005 | 326 | 6 | 0 | 6 | 353617.6 | 0.16603107 | 1.00 |
| X | hemizygous | GPKOW | 1005 | 326 | 5 | 0 | 5 | 404286 | 0.16650324 | 1.00 |
| X | hemizygous | NYX | 1005 | 326 | 6 | 0 | 6 | 378487.8 | 0.18030528 | 1.00 |
| X | hemizygous | PPEF1 | 1005 | 326 | 5 | 0 | 5 | 346961.5 | 0.1865711 | 1.00 |
| X | hemizygous | RAI2 | 1005 | 326 | 6 | 0 | 6 | 279086.7 | 0.19150002 | 1.00 |
| X | hemizygous | WWC3 | 1005 | 326 | 8 | 4 | 12 | 0.388992 | 0.19366462 | 1.00 |
| X | hemizygous | CA5B | 1005 | 326 | 3 | 3 | 6 | 0.294886 | 0.19446182 | 1.00 |
| X | hemizygous | MID2 | 1005 | 326 | 5 | 0 | 5 | 303835.7 | 0.20459628 | 1.00 |
| X | hemizygous | FAM155B | 1005 | 326 | 5 | 0 | 5 | 415820.8 | 0.20655915 | 1.00 |
| X | hemizygous | DOCK11 | 1005 | 326 | 9 | 1 | 10 | 3.784524 | 0.2131783 | 1.00 |
| X | hemizygous | NUP62CL | 1005 | 326 | 5 | 0 | 5 | 286096.2 | 0.21991501 | 1.00 |
| X | hemizygous | FAM47C | 1005 | 326 | 5 | 4 | 9 | 0.420001 | 0.22291489 | 1.00 |
| X | hemizygous | PCDH19 | 1005 | 326 | 3 | 3 | 6 | 0.336221 | 0.22475384 | 1.00 |
| X | hemizygous | CCNB3 | 1005 | 326 | 10 | 1 | 11 | 3.814577 | 0.22760231 | 1.00 |
| X | hemizygous | HDAC6 | 1005 | 326 | 10 | 1 | 11 | 3.405368 | 0.23434086 | 1.00 |
| X | hemizygous | SYN1 | 1005 | 326 | 5 | 0 | 5 | 270419.4 | 0.24062233 | 1.00 |
| X | hemizygous | HCFC1 | 1005 | 326 | 6 | 0 | 6 | 229826.7 | 0.25114382 | 1.00 |
| X | hemizygous | JADE3 | 1005 | 326 | 6 | 0 | 6 | 223588.8 | 0.25329687 | 1.00 |
| X | hemizygous | UBQLN2 | 1005 | 326 | 5 | 0 | 5 | 243439.1 | 0.25378852 | 1.00 |
| X | hemizygous | ZNF157 | 1005 | 326 | 4 | 3 | 7 | 0.357525 | 0.25680374 | 1.00 |
| X | hemizygous | NRK | 1005 | 326 | 9 | 2 | 11 | 2.494375 | 0.25760088 | 1.00 |
| X | hemizygous | RBMXL3 | 1005 | 326 | 13 | 6 | 19 | 0.522073 | 0.27150687 | 1.00 |
| X | hemizygous | ZMYM3 | 1005 | 326 | 4 | 3 | 7 | 0.35163 | 0.27397319 | 1.00 |
| X | hemizygous | TLR8 | 1005 | 326 | 7 | 0 | 7 | 451502.6 | 0.27483408 | 1.00 |
| X | hemizygous | DRP2 | 1005 | 326 | 5 | 4 | 9 | 0.432671 | 0.27626051 | 1.00 |
| X | hemizygous | TXLNG | 1005 | 326 | 3 | 2 | 5 | 0.312461 | 0.28028089 | 1.00 |
| X | hemizygous | AVPR2 | 1005 | 326 | 3 | 2 | 5 | 0.313205 | 0.28366088 | 1.00 |
| X | hemizygous | KIAA2022 | 1005 | 326 | 8 | 1 | 9 | 3.189054 | 0.28630685 | 1.00 |
| X | hemizygous | ARHGAP6 | 1005 | 326 | 6 | 5 | 11 | 0.4855 | 0.29058332 | 1.00 |
| X | hemizygous | SAGE1 | 1005 | 326 | 9 | 1 | 10 | 2.911813 | 0.3018465 | 1.00 |
| X | hemizygous | SHROOM2 | 1005 | 326 | 11 | 6 | 17 | 0.553965 | 0.31095801 | 1.00 |
| X | hemizygous | PASD1 | 1005 | 326 | 5 | 2 | 7 | 0.363449 | 0.3112386 | 1.00 |
| X | hemizygous | DMD | 1005 | 326 | 24 | 10 | 34 | 0.646627 | 0.32126553 | 1.00 |
| X | hemizygous | TKTL1 | 1005 | 326 | 9 | 1 | 10 | 2.678586 | 0.32544947 | 1.00 |
| X | hemizygous | TRO | 1005 | 326 | 15 | 2 | 17 | 2.163384 | 0.32596703 | 1.00 |
| X | hemizygous | ZNF185 | 1005 | 326 | 4 | 3 | 7 | 0.361882 | 0.3270074 | 1.00 |
| X | hemizygous | RAB41 | 1005 | 326 | 5 | 1 | 6 | 2.891164 | 0.3286014 | 1.00 |
| X | hemizygous | CT47B1 | 1005 | 326 | 6 | 4 | 10 | 0.480174 | 0.32866378 | 1.00 |
| X | hemizygous | MAP7D2 | 1005 | 326 | 6 | 1 | 7 | 3.184667 | 0.3299843 | 1.00 |
| X | hemizygous | STAG2 | 1005 | 326 | 3 | 2 | 5 | 0.363508 | 0.3347039 | 1.00 |
| X | hemizygous | MED12 | 1005 | 326 | 4 | 3 | 7 | 0.432211 | 0.33662685 | 1.00 |
| X | hemizygous | STS | 1005 | 326 | 4 | 2 | 6 | 0.391282 | 0.34113839 | 1.00 |
| X | hemizygous | TBC1D8B | 1005 | 326 | 7 | 1 | 8 | 2.695813 | 0.34217652 | 1.00 |
| X | hemizygous | CSAG1 | 1005 | 326 | 5 | 0 | 5 | 164755.8 | 0.34866045 | 1.00 |
| X | hemizygous | ARMCX1 | 1005 | 326 | 4 | 1 | 5 | 2.906655 | 0.35452925 | 1.00 |
| X | hemizygous | PCDH11X | 1005 | 326 | 7 | 1 | 8 | 2.572298 | 0.35.51925 | 1.00 |
| X | hemizygous | DDX26B | 1005 | 326 | 3 | 3 | 6 | 0.445161 | 0.36020105 | 1.00 |
| X | hemizygous | KIAA1210 | 1005 | 326 | 14 | 2 | 16 | 2.048898 | 0.37620773 | 1.00 |
| X | hemizygous | WNK3 | 1005 | 326 | 7 | 4 | 11 | 0.546651 | 0.38071767 | 1.00 |
| X | hemizygous | HEPH | 1005 | 326 | 8 | 1 | 9 | 2.658925 | 0.38855647 | 1.00 |
| X | hemizygous | ATP2B3 | 1005 | 326 | 11 | 5 | 16 | 0.578172 | 0.38937888 | 1.00 |
| X | hemizygous | KDM5C | 1005 | 326 | 8 | 1 | 9 | 2.340347 | 0.40002574 | 1.00 |
| X | hemizygous | CNGA2 | 1005 | 326 | 7 | 2 | 9 | 0.474466 | 0.4002039 | 1.00 |
| X | hemizygous | DIAPH2 | 1005 | 326 | 3 | 2 | 5 | 0.39439 | 0.41082511 | 1.00 |
| X | hemizygous | FRMPD4 | 1005 | 326 | 5 | 4 | 9 | 0.536334 | 0.41205848 | 1.00 |
| X | hemizygous | LANCL3 | 1005 | 326 | 4 | 2 | 6 | 0.464615 | 0.41459917 | 1.00 |
| X | hemizygous | CXorf30 | 1005 | 326 | 6 | 1 | 7 | 2.518132 | 0.42113484 | 1.00 |
| X | hemizygous | SYTLA | 1005 | 326 | 4 | 3 | 7 | 0.499913 | 0.42476417 | 1.00 |
| X | hemizygous | ARHGEF6 | 1005 | 326 | 3 | 2 | 5 | 0.408192 | 0.42643361 | 1.00 |
| X | hemizygous | MAGEC1 | 1005 | 326 | 10 | 2 | 12 | 2.196884 | 0.42721386 | 1.00 |
| X | hemizygous | AR | 1005 | 326 | 5 | 1 | 6 | 2.622802 | 0.43031031 | 1.00 |
| X | hemizygous | PHKA2 | 1005 | 326 | 9 | 1 | 10 | 2.151619 | 0.43462731 | 1.00 |
| X | hemizygous | GPR50 | 1005 | 326 | 9 | 2 | 11 | 1.835733 | 0.43518962 | 1.00 |
| X | hemizygous | PLS3 | 1005 | 326 | 5 | 1 | 6 | 0.383123 | 0.45145638 | 1.00 |
| X | hemizygous | FAM47A | 1005 | 326 | 5 | 3 | 8 | 0.525323 | 0.45213641 | 1.00 |
| X | hemizygous | BGN | 1005 | 326 | 4 | 1 | 5 | 2.257106 | 0.45594668 | 1.00 |
| X | hemizygous | ACOT9 | 1005 | 326 | 5 | 2 | 7 | 0.48439 | 0.46087866 | 1.00 |
| X | hemizygous | MORC4 | 1005 | 326 | 9 | 1 | 7 | 2.107941 | 0.46538034 | 1.00 |
| X | hemizygous | CXorf64 | 1005 | 326 | 7 | 1 | 8 | 2.085247 | 0.46815704 | 1.00 |
| X | hemizygous | OPHN1 | 1005 | 326 | 8 | 1 | 9 | 2.060293 | 0.4779888 | 1.00 |
| X | hemizygous | BRWD3 | 1005 | 326 | 5 | 1 | 6 | 2.129876 | 0.48049255 | 1.00 |
| X | hemizygous | MAP3K15 | 1005 | 326 | 9 | 4 | 13 | 0.610618 | 0.4828055 | 1.00 |
| X | hemizygous | MAGEE1 | 1005 | 326 | 7 | 1 | 8 | 2.048651 | 0.48856106 | 1.00 |
| X | hemizygous | GPRASP2 | 1005 | 326 | 4 | 2 | 6 | 2.100594 | 0.49229769 | 1.00 |
| X | hemizygous | AMER1 | 1005 | 326 | 8 | 1 | 9 | 2.059217 | 0.49509022 | 1.00 |
| X | hemizygous | FAAH2 | 1005 | 326 | 6 | 1 | 7 | 2.324614 | 0.5146221 | 1.00 |
| X | hemizygous | MTM1 | 1005 | 326 | 3 | 2 | 5 | 0.525404 | 0.54511099 | 1.00 |
| X | hemizygous | AIFM1 | 1005 | 326 | 4 | 2 | 6 | 0.561051 | 0.54569924 | 1.00 |
| X | hemizygous | XG | 1005 | 326 | 6 | 1 | 7 | 1.934616 | 0.55349695 | 1.00 |
| X | hemizygous | UBA1 | 1005 | 326 | 11 | 3 | 14 | 1.460233 | 0.57054421 | 1.00 |
| X | hemizygous | CFP | 1005 | 326 | 3 | 2 | 5 | 0.565234 | 0.57056967 | 1.00 |
| X | hemizygous | MED14 | 1005 | 326 | 4 | 2 | 6 | 0.542082 | 0.57667526 | 1.00 |
| X | hemizygous | TENM1 | 1005 | 326 | 9 | 4 | 13 | 1.43647 | 0.57778215 | 1.00 |
| X | hemizygous | FANCB | 1005 | 326 | 4 | 2 | 6 | 0.572135 | 0.58193907 | 1.00 |
| X | hemizygous | TSPYL2 | 1005 | 326 | 3 | 2 | 5 | 0.570147 | 0.58628811 | 1.00 |
| X | hemizygous | CACNA1F | 1005 | 326 | 14 | 2 | 16 | 1.523641 | 0.5865024 | 1.00 |
| X | hemizygous | MECP2 | 1005 | 326 | 4 | 1 | 5 | 0.490915 | 0.58954833 | 1.00 |
| X | hemizygous | MUM1L1 | 1005 | 326 | 6 | 1 | 7 | 1.82059 | 0.58993327 | 1.00 |
| X | hemizygous | PLXNA3 | 1005 | 326 | 18 | 8 | 26 | 0.769418 | 0.59125032 | 1.00 |
| X | hemizygous | CXorf22 | 1005 | 326 | 10 | 2 | 12 | 1.55994 | 0.59152013 | 1.00 |
| X | hemizygous | ASB12 | 1005 | 326 | 4 | 1 | 5 | 1.892128 | 0.60060341 | 1.00 |
| X | hemizygous | ARHGAP4 | 1005 | 326 | 7 | 3 | 10 | 0.662336 | 0.60557195 | 1.00 |
| X | hemizygous | SLC7A3 | 1005 | 326 | 7 | 1 | 8 | 1.820953 | 0.60925859 | 1.00 |
| X | hemizygous | CCDC22 | 1005 | 326 | 6 | 3 | 9 | 0.68009 | 0.61792786 | 1.00 |
| X | hemizygous | KIF4A | 1005 | 326 | 8 | 2 | 10 | 1.53.5086 | 0.61823222 | 1.00 |
| X | hemizygous | ATP7A | 1005 | 326 | 7 | 2 | 9 | 0.638615 | 0.61920744 | 1.00 |
| X | hemizygous | OFD1 | 1005 | 326 | 4 | 1 | 5 | 1.764963 | 0.62501146 | 1.00 |
| X | hemizygous | CYSLTR1 | 1005 | 326 | 5 | 1 | 6 | 1.715937 | 0.62678485 | 1.00 |
| X | hemizygous | TFDP3 | 1005 | 326 | 4 | 1 | 5 | 0.514022 | 0.62877181 | 1.00 |
| X | hemizygous | ARAF | 1005 | 326 | 3 | 2 | 5 | 0.613673 | 0.63518649 | 1.00 |
| X | hemizygous | CYBB | 1005 | 326 | 4 | 1 | 5 | 1.773761 | 0.63885256 | 1.00 |
| X | hemizygous | MTMR1 | 1005 | 326 | 6 | 1 | 7 | 1.661729 | 0.65002017 | 1.00 |
| X | hemizygous | ALG13 | 1005 | 326 | 5 | 2 | 7 | 1.685876 | 0.65940118 | 1.00 |
| X | hemizygous | MAGIX | 1005 | 326 | 4 | 1 | 5 | 1.736425 | 0.6594296 | 1.00 |
| X | hemizygous | CTAG2 | 1005 | 326 | 4 | 1 | 5 | 1.593603 | 0.67768793 | 1.00 |
| X | hemizygous | KDM6A | 1005 | 326 | 4 | 1 | 5 | 1.591958 | 0.68331002 | 1.00 |
| X | hemizygous | P2RY4 | 1005 | 326 | 4 | 1 | 5 | 0.556881 | 0.68992064 | 1.00 |
| X | hemizygous | CXorf66 | 1005 | 326 | 4 | 1 | 5 | 0.614649 | 0.70381454 | 1.00 |
| X | hemizygous | PLXNB3 | 1005 | 326 | 17 | 5 | 22 | 0.807878 | 0.70586385 | 1.00 |
| X | hemizygous | ITIH6 | 1005 | 326 | 14 | 3 | 17 | 1.31289 | 0.70747903 | 1.00 |
| X | hemizygous | ZNF630 | 1005 | 326 | 5 | 1 | 6 | 0.621221 | 0.7133272 | 1.00 |
| X | hemizygous | SYTL5 | 1005 | 326 | 3 | 2 | 5 | 0.692525 | 0.71529137 | 1.00 |
| X | hemizygous | GPRASP1 | 1005 | 326 | 7 | 3 | 10 | 0.763285 | 0.72312408 | 1.00 |
| X | hemizygous | ZXDA | 1005 | 326 | 7 | 1 | 8 | 1.447006 | 0.73151986 | 1.00 |
| X | hemizygous | KLHL4 | 1005 | 326 | 4 | 2 | 6 | 0.714062 | 0.73752215 | 1.00 |
| X | hemizygous | ESX1 | 1005 | 326 | 6 | 1 | 7 | 1.489758 | 0.74006001 | 1.00 |
| X | hemizygous | EGFL6 | 1005 | 326 | 6 | 1 | 7 | .460673 | 0.74168947 | 1.00 |
| X | hemizygous | SRPK | 1005 | 326 | 5 | 1 | 6 | 1.43459 | 0.7491792 | 1.00 |
| X | hemizygous | FAM47B | 1005 | 326 | 7 | 4 | 11 | 0.778583 | 0.74923042 | 1.00 |
| X | hemizygous | IGSF1 | 1005 | 326 | 9 | 3 | 12 | 1.245574 | 0.75769672 | 1.00 |
| X | hemizygous | GDPD2 | 1005 | 326 | 3 | 2 | 5 | 0.731719 | 0.76014818 | 1.00 |
| X | hemizygous | VSIG4 | 1005 | 326 | 8 | 5 | 13 | 0.830279 | 0.76559025 | 1.00 |
| X | hemizygous | HTATSF1 | 1005 | 326 | 3 | 2 | 5 | 0.76151 | 0.77485397 | 1.00 |
| X | hemizygous | CHRDL1 | 1005 | 326 | 4 | 2 | 6 | 0.746524 | 0.77772466 | 1.00 |
| X | hemizygous | HUWE1 | 1005 | 326 | 8 | 1 | 9 | 1.340224 | 0.78394567 | 1.00 |
| X | hemizygous | MAGEA4 | 1005 | 326 | 4 | 2 | 6 | 1.259601 | 0.79649927 | 1.00 |
| X | hemizygous | MAGEB16 | 1005 | 326 | 5 | 2 | 7 | 1.350371 | 0.80445789 | 1.00 |
| X | hemizygous | BCORL1 | 1005 | 326 | 14 | 5 | 19 | 1.163272 | 0.81640999 | 1.00 |
| X | hemizygous | F8 | 1005 | 326 | 8 | 2 | 10 | 1.206669 | 0.81761474 | 1.00 |
| X | hemizygous | ARMCX4 | 1005 | 326 | 15 | 5 | 20 | 1.129638 | 0.8270148 | 1.00 |
| X | hemizygous | ARSH | 1005 | 326 | 5 | 1 | 6 | 1.28334 | 0.82933282 | 1.00 |
| X | hemizygous | COL4A6 | 1005 | 326 | 6 | 1 | 7 | 0.790176 | 0.83599068 | 1.00 |
| X | hemizygous | GABRQ | 1005 | 326 | 7 | 1 | 8 | 1.249699 | 0.83832641 | 1.00 |
| X | hemizygous | AKAP4 | 1005 | 326 | 4 | 1 | 5 | 1.258002 | 0.84058544 | 1.00 |
| X | hemizygous | NOX1 | 1005 | 326 | 5 | 2 | 7 | 0.829308 | 0.84067036 | 1.00 |
| X | hemizygous | SLC6A8 | 1005 | 326 | 5 | 3 | 8 | 0.842072 | 0.8430802 | 1.00 |
| X | hemizygous | IDS | 1005 | 326 | 5 | 1 | 6 | 1.229301 | 0.85278486 | 1.00 |
| X | hemizygous | DACH2 | 1005 | 326 | 5 | 1 | 6 | 1.21834 | 0.85890014 | 1.00 |
| X | hemizygous | MAGEB2 | 1005 | 326 | 4 | 1 | 5 | 0.818602 | 0.86761712 | 1.00 |
| X | hemizygous | SSX1 | 1005 | 326 | 4 | 1 | 5 | 1.192693 | 0.87399789 | 1.00 |
| X | hemizygous | GUCY2F | 1005 | 326 | 9 | 2 | 11 | 1.13039 | 0.88537594 | 1.00 |
| X | hemizygous | PDZD4 | 1005 | 326 | 4 | 1 | 5 | 1.175565 | 0.89086852 | 1.00 |
| X | hemizygous | GPR64 | 1005 | 326 | 6 | 1 | 7 | 0.861971 | 0.89616577 | 1.00 |
| X | hemizygous | TEX11 | 1005 | 326 | 6 | 2 | 8 | 0.886165 | 0.89929157 | 1.00 |
| X | hemizygous | CLCN5 | 1005 | 326 | 5 | 1 | 6 | 0.858541 | 0.90303952 | 1.00 |
| X | hemizygous | APEX2 | 1005 | 326 | 6 | 3 | 9 | 0.91144 | 0.9132655 | 1.00 |
| X | hemizygous | STARD8 | 1005 | 326 | 9 | 2 | 11 | 0.913632 | 0.91750323 | 1.00 |
| X | hemizygous | NHS | 1005 | 326 | 7 | 2 | 9 | 0.935201 | 0.94075109 | 1.00 |
| X | hemizygous | MAMLD1 | 1005 | 326 | 14 | 3 | 17 | 0.957557 | 0.94994294 | 1.00 |
| X | hemizygous | SLC25A53 | 1005 | 326 | 5 | 1 | 6 | 1.073034 | 0.9502513 | 1.00 |
| X | hemizygous | PRICKLE3 | 1005 | 326 | 7 | 2 | 9 | 1.053727 | 0.95376192 | 1.00 |
| X | hemizygous | FLNA | 1005 | 326 | 14 | 6 | 20 | 1.030302 | 0.95659113 | 1.00 |
| X | hemizygous | USP26 | 1005 | 326 | 7 | 1 | 8 | 1.049564 | 0.96725269 | 1.00 |
| X | hemizygous | ATP6AP1 | 1005 | 326 | 4 | 1 | 5 | 1.047538 | 0.96944855 | 1.00 |
| X | hemizygous | XPNPEP2 | 1005 | 326 | 5 | 2 | 7 | 1.035467 | 0.97194181 | 1.00 |
| X | hemizygous | MXRA5 | 1005 | 326 | 22 | 5 | 27 | 1.014729 | 0.97829312 | 1.00 |
| X | hemizygous | MAGED1 | 1005 | 326 | 4 | 1 | 5 | 1.030381 | 0.98007573 | 1.00 |
| X | hemizygous | SMARCA1 | 1005 | 326 | 4 | 1 | 5 | 0.975903 | 0.98339618 | 1.00 |

TABLE 14
Statistical analysis of non-synonymous TLR7 and TLR8 rare variants in our cohorts
Coding non-synonymous (MAF < 10-4)

| | Critical | | | Asymptomatic | | | |
|---|---|---|---|---|---|---|---|
| | Individuals | Individuals | | Individuals | Individuals with | | |
| | with rare or | with no rare | | with rare or | no rare or | | |
| | private | or private | total | private | private | total | P |
| Gene | variants | variant | individuals | variants | variant | individuals | value |
| TLR7 | 19 | 986 | 1005 | 0 | 326 | 326 | 6.0E−06 |
| TLR8 | 7 | 998 | 1005 | 0 | 326 | 326 | 0.27 |

TABLE 15
TLR7 variant activity reported in this study, in previous studies and in gnomAD (v2.1.1)

| | | | | | | | | | | NF-KB | | | | | | | | | | | | | |
|---|---|---|---|---|---|---|---|---|---|---|---|---|---|---|---|---|---|---|---|---|---|---|---|
| | | | | | | | | Tran- | | activity | | | | | | | | | | | | Cumu- | Cumu- |
| | Protein | | | | | | | script | | mean | NF-KB activity | | | | | Allele | Allele | Homo- | Hemi- | hemi- | AF | lative | lative |
| | Conse- | Chromo- | | | Ref- | Alter- | | Conse- | Expres- | (% of | (% of WT) | | | | Allele | Num- | Fre- | zygote | zygote | zygote | hemi- | MAF | MAF |
| Source | quence | some | Position | [symbol] | erence | nate | Annotation | quence | sion | WT) | #1 | #2 | #3 | #4 | Count | ber | quency | Count | Count | total | zygous | LDF | [symbol] |
| In-house | [symbol] | X | [symbol] | — | T | C | missense | c. [symbol] | normal | 2 | 1 | 0 | 0 | 5 | — | — | — | — | — | — | — | | |
| cohort | | | | | | | | | | | | | | | | | | | | | | | |
| In-house | | X | | — | GC | G | indel- | | no | 0 | 1 | 0 | 0 | 1 | — | — | — | — | — | — | — | | |
| cohort | | | | | | | frameshift | | | | | | | | | | | | | | | | |
| In-house | | X | | — | CT | C | indel- | | no | 0 | 0 | 1 | 0 | 1 | — | — | — | — | — | — | — | | |
| cohort | | | | | | | frameshift | | | | | | | | | | | | | | | | |
| In-house | | X | | — | T | G | missense | | normal | 105 | 80 | 87 | 123 | 118 | — | — | — | — | — | — | — | | |
| cohort | | | | | | | | | | | | | | | | | | | | | | | |
| In-house | | X | | — | G | T | missense | | normal | 0 | 0 | 0 | 1 | 0 | — | — | — | — | — | — | — | | |
| cohort | | | | | | | | | | | | | | | | | | | | | | | |
| In-house | | X | | — | A | C | missense | | normal | 47 | 53 | 50 | 44 | 42 | — | — | — | — | — | — | — | | |
| cohort | | | | | | | | | | | | | | | | | | | | | | | |
| In-house | | X | | — | T | C | missense | | normal | 0 | 0 | 0 | 0 | 0 | — | — | — | — | — | — | — | | |
| cohort | | | | | | | | | | | | | | | | | | | | | | | |
| In-house | | X | | — | T | C | missense | | normal | 0 | 1 | 0 | 0 | 0 | — | — | — | — | — | — | — | | |
| cohort | | | | | | | | | | | | | | | | | | | | | | | |
| In-house | | X | | rs [symbol] | A | C | missense | | normal | 32 | 136 | 111 | 77 | 72 | 5 | [symbol] | 2.7E−05 | 0 | 3 | 87680 | 4.4E−05 | | |
| cohort | | | | | | | | | | | | | | | | | | | | | | | |
| In-house | | X | | — | C | T | missense | | normal | 1 | 1 | 0 | 2 | 3 | — | — | — | — | — | — | — | | |
| cohort | | | | | | | | | | | | | | | | | | | | | | | |
| In-house | | X | | — | G | A | missense | | normal | 96 | 56 | 64 | 82 | 80 | — | — | — | — | — | — | — | | |
| cohort | | | | | | | | | | | | | | | | | | | | | | | |
| In-house | | X | | — | T | C | missense | | normal | 8 | 8 | 8 | 8 | 13 | — | — | — | — | — | — | — | | |
| cohort | | | | | | | | | | | | | | | | | | | | | | | |
| In-house | | X | | — | C | T | missense | | normal | 5 | 7 | 13 | 8 | 7 | — | — | — | — | — | — | — | | |
| cohort | | | | | | | | | | | | | | | | | | | | | | | |
| In-house | | X | | — | A | T | [symbol] | | normal | 0 | 0 | 0 | 1 | 0 | — | — | — | — | — | — | — | | |
| cohort | | | | | | | | | | | | | | | | | | | | | | | |
| In-house | | X | | — | A | T | missense | | normal | 1 | 1 | 0 | 0 | 1 | — | — | — | — | — | — | — | | |
| cohort | | | | | | | | | | | | | | | | | | | | | | | |
| In-house | | X | | rs [symbol] | G | A | missense | | normal | 122 | 130 | 124 | 125 | 110 | 2 | 182706 | [symbol] | 0 | 1 | 67377 | 1.5E−05 | | |
| cohort | | | | | | | | | | | | | | | | | | | | | | | |
| In-house | | X | | rs [symbol] | T | C | missense | | normal | 2 | 3 | 2 | 0 | 3 | 10 | [symbol] | 5.6E−05 | 0 | 5 | 67812 | 7.4E−05 | | |
| cohort | | | | | | | | | | | | | | | | | | | | | | | |
| In-house | | X | | rs [symbol] | G | A | missense | | normal | 2 | 3 | 2 | 0 | 3 | — | — | — | — | — | — | — | | |
| cohort | | | | | | | | | | | | | | | | | | | | | | | |
| cAMA | | X | | — | CAACT | C | indel- | | normal | 1 | 1 | 1 | 4 | 0 | — | — | — | — | — | — | — | | |
| | | | | | | | frameshift | | | | | | | | | | | | | | | | |
| cAMA | | X | | — | G | T | missense | | normal | 1 | 0 | 0 | 3 | 1 | — | — | — | — | — | — | — | | |
| eife | | X | | — | T | C | missense | | normal | 0 | 0 | 0 | 0 | 0 | — | — | — | — | — | — | — | | |
| eife | | X | | | C | T | missense | | normal | 21 | 20 | 22 | 21 | 25 | 3 | 183285 | 1.5E−05 | 0 | 1 | | 1.5E−05 | | |
| eife | | X | | | G | A | missense | | normal | 83 | 53 | 80 | 83 | 84 | 542 | 205624 | 4.6E−03 | 11 | 236 | 75678 | 3.1E−03 | | |
| eife | | X | | | C | T | missense | | normal | 108 | 122 | 103 | 102 | 103 | [symbol] | 202954 | 3.0E−03 | 3 | 206 | 74658 | 2.8E−03 | | |
| eife | | X | | | G | A | missense | | normal | 30 | 73 | 94 | [symbol] | 105 | 67 | 183281 | 3.7E−04 | 0 | 13 | 67787 | 2.6E−04 | | |
| eife | | X | | | G | A | missense | | normal | 81 | 42 | 43 | | 67 | 136 | [symbol] | 6.7E−04 | 2 | 50 | 74653 | 6.7E−04 | | |
| gromAD | | X | | | A | T | missense | | normal | 113 | 125 | 123 | 100 | 105 | 35365 | 200580 | 1.8E−01 | 2446 | 12833 | 71478 | 1.8E−01 | | |
| gromAD | | X | | | G | A | missense | | normal | 33 | [symbol] | [symbol] | | 84 | 942 | 205024 | 4.6E−03 | 11 | 236 | 75676 | 3.1E−03 | | |
| gromAD | | X | | | C | T | missense | | normal | 105 | 122 | 107 | | [symbol] | | 202954 | 3.0E−03 | 3 | 256 | [symbol] | 2.8E−03 | | |
| gromAD | | X | | | T | A | missense | | normal | 105 | 111 | 121 | 98 | 105 | | | 2.5E−03 | 3 | 169 | | 2.5E−03 | | |
| gromAD | | X | | | G | A | missense | | normal | 81 | 42 | 43 | 93 | 87 | 136 | 204458 | 6.7E−04 | 2 | 50 | 75069 | 6.7E−04 | | |
| gromAD | | X | | | G | A | missense | | normal | [symbol] | 73 | 94 | 25 | 100 | | 183281 | 3.7E−04 | 0 | 19 | 67787 | 2.8E−04 | | |
| gromAD | | X | | | G | A | missense | | normal | 116 | 157 | 102 | | 138 | 37 | 205239 | 1.6E−04 | 0 | 13 | | 1.7E−04 | | |
| gromAD | | X | | | C | T | missense | | normal | 77 | 54 | 76 | 77 | 91 | 28 | 205001 | 1.8E−04 | 0 | 9 | 75623 | 1.2E−04 | | |
| gromAD | | X | | | C | T | missense | | normal | 58 | 35 | 81 | 78 | 53 | 28 | 205087 | 1.3E−04 | 0 | 8 | 75629 | 7.9E−05 | | |
| gromAD | | X | | | A | G | missense | | normal | 13 | 10 | 13 | 10 | 17 | 23 | 205236 | 1.1E−04 | 0 | 10 | | 1.3E−04 | | 1.3E−04 |
| gromAD | | X | | | A | G | missense | | normal | 114 | 141 | 137 | | 100 | 21 | 204456 | 1.0E−04 | 0 | 3 | 75232 | 4.0E−05 | | |
| gromAD | | X | | | A | G | missense | | no | 6 | 8 | 5 | 4 | 5 | 2 | 25132 | 9.0E−04 | 0 | 1 | 34388 | 2.9E−05 | | 2.9E−05 |
| gromAD | | X | | | A | G | missense | | normal | 85 | 57 | 77 | 61 | 85 | 12 | 182718 | 6.6E−05 | 6 | 7 | | 1.0E−04 | | |
| gromAD | | X | | | G | A | missense | | normal | 104 | 95 | 107 | 101 | 114 | 10 | 182002 | 5.5E−05 | 6 | 2 | 86730 | 3.0E−05 | | |
| gromAD | | X | | | T | C | missense | | normal | 2 | 3 | 2 | 0 | 3 | 13 | 183870 | 5.5E−05 | 6 | 5 | 87812 | 7.4E−05 | 7.4E−05 | |
| gromAD | | X | | | A | T | missense | | normal | 70 | | | 58 | 73 | 1 | 21973 | 4.6E−05 | 6 | 1 | 24302 | 2.8E−05 | | |
| gromAD | | X | | | A | G | missense | | normal | 2 | 1 | | 0 | 3 | 1 | 22042 | 4.5E−05 | 6 | 1 | 34302 | 2.9E−05 | 2.9E−05 | |
| gromAD | | X | | | C | T | missense | | normal | 85 | | 77 | 61 | 56 | 8 | 181731 | 4.4E−05 | 6 | 2 | | 3.0E−05 | | |
| gromAD | | X | | | G | C | missense | | normal | 15 | 13 | 15 | 18 | 17 | 8 | 204853 | 3.8E−05 | 6 | 2 | 75587 | 2.8E−05 | | 2.8E−05 |
| gromAD | | X | | | C | A | missense | | normal | 5 | 2 | 8 | 10 | 1 | 8 | 205129 | 3.8E−05 | 6 | 2 | 75857 | 2.8E−05 | | 2.8E−05 |
| gromAD | | X | | | T | C | missense | | normal | 92 | | 95 | | 93 | 7 | 183895 | 3.8E−05 | 6 | 4 | 67867 | 5.8E−05 | | |
| gromAD | | X | | | G | A | missense | | normal | 74 | 71 | 65 | | 78 | 7 | 183437 | 3.8E−05 | 6 | 4 | 67885 | 5.8E−05 | | |
| gromAD | | X | | | T | C | missense | | normal | 33 | 58 | 93 | 68 | 94 | 7 | 183037 | 3.8E−05 | 6 | 2 | 87847 | 3.0E−05 | | |
| gromAD | | X | | | A | T | missense | | normal | 105 | 82 | 95 | 105 | 135 | 6 | 181113 | 3.3E−05 | 6 | 2 | | 3.0E−05 | | |
| gromAD | | X | | | C | T | missense | | normal | 77 | 77 | 85 | 88 | 77 | 6 | 183702 | 3.3E−05 | 6 | 6 | 67372 | 5.8E−05 | | |
| gromAD | | X | | | G | C | missense | | normal | 100 | 31 | 105 | 101 | 113 | 6 | 183136 | 3.3E−05 | 6 | 4 | | 5.8E−05 | | |
| gromAD | | X | | | C | T | missense | | normal | 58 | 47 | 58 | 58 | 69 | 6 | 187228 | 3.3E−05 | 0 | 1 | 63418 | 1.6E−05 | | |
| gromAD | | X | | | G | A | missense | | normal | 97 | 118 | 193 | 78 | 92 | 6 | 204874 | 2.9E−05 | 0 | 2 | | 2.8E−05 | | |
| gromAD | | X | | | G | A | missense | | low | 117 | 117 | 131 | | 113 | 6 | 205151 | 2.9E−05 | 0 | 2 | 75796 | 2.8E−05 | | |
| gromAD | | X | | | G | C | missense | | low | | 81 | 51 | 83 | 63 | 6 | | 2.9E−05 | 0 | 1 | | 1.3E−05 | | |
| gromAD | | X | | | A | T | missense | | normal | 102 | 85 | 107 | 99 | 117 | 5 | 183187 | 2.7E−05 | 0 | 4 | 67703 | 5.9E−05 | | |
| gromAD | | X | | | T | A | missense | | normal | 110 | 99 | 101 | 117 | 123 | 5 | 183051 | 2.7E−05 | 0 | 3 | | 4.4E−05 | | |
| gromAD | | X | | | A | C | missense | | normal | 92 | 108 | 111 | 77 | 72 | 5 | 183154 | 2.7E−05 | 0 | 3 | 67850 | 4.4E−05 | | |
| gromAD | | X | | | C | A | missense | | normal | 95 | 32 | 88 | 105 | 87 | 5 | 183242 | 2.7E−05 | 0 | 3 | 67734 | 4.4E−05 | | |
| gromAD | | X | | | C | A | missense | | normal | 0 | 0 | 0 | 0 | 0 | 5 | 183291 | 2.7E−05 | 0 | 3 | 67777 | 4.4E−05 | 4.4E−05 | |
| gromAD | | X | | | A | T | missense | | low | 58 | 35 | 167 | 36 | 100 | 5 | | 2.7E−05 | 0 | 2 | 57764 | 3.0E−06 | | |
| gromAD | | X | | | T | C | missense | | low | | 46 | 53 | | 54 | 5 | 204925 | 2.4E−05 | 0 | 1 | 75865 | 1.3E−06 | | |
| gromAD | | X | | | C | T | missense | | normal | 66 | 81 | 61 | 52 | 72 | 4 | 162742 | 2.2E−05 | 0 | 1 | 67424 | 1.5E−06 | | |
| gromAD | | X | | | C | T | missense | | low | 76 | 73 | 68 | 66 | 71 | 4 | | 2.2E−05 | 0 | 2 | 67767 | 3.0E−06 | | |
| gromAD | | X | | | A | C | missense | | low | 52 | | 57 | 45 | | 4 | | 2.2E−05 | 0 | 2 | | [symbol] | | |
| gromAD | | X | | | C | T | missense | | low | 5 | 8 | 6 | 5 | 4 | 4 | | 2.0E−05 | 0 | 2 | | 2.7E−06 | | 2.7E−06 |
| gromAD | | X | | | A | G | missense | | low | 60 | | 65 | 53 | 72 | 4 | | 2.0E−05 | 0 | 2 | | 2.6E−06 | | |
| gromAD | | X | | | T | C | missense | | normal | 121 | 103 | 141 | 117 | 123 | 3 | | 1.7E−05 | 0 | 1 | | 1.6E−06 | | |
| gromAD | | X | | | G | A | missense | | normal | 52 | 84 | 65 | | 132 | 3 | | 1.7E−05 | 0 | 1 | | 1.5E−06 | | |
| gromAD | | X | | | T | G | missense | | normal | 199 | 96 | 152 | 88 | 102 | 3 | | 1.6E−05 | 0 | 2 | 67401 | 3.0E−06 | | |
| gromAD | | X | | | CCTA | C | [symbol] | | normal | 194 | | 128 | 74 | 154 | 3 | | 1.6E−05 | 0 | 2 | 67586 | 3.0E−06 | | |
| gromAD | | X | | | A | G | missense | | normal | 7 | 8 | 10 | 5 | 7 | 3 | | 1.6E−05 | 0 | 2 | 67786 | 3.0E−06 | | 3.0E−06 |
| gromAD | | X | | | C | T | missense | | normal | 21 | 20 | 22 | | 25 | 3 | 183365 | 1.6E−05 | 0 | 1 | 67811 | 1.5E−06 | | 1.5E−06 |
| gromAD | | X | | | T | C | missense | | normal | 6 | 4 | 6 | 5 | 9 | 3 | | 1.5E−05 | 0 | 2 | 75833 | 2.6E−06 | | 2.6E−06 |
| gromAD | | X | | | A | T | missense | | normal | 167 | 156 | 130 | | 132 | 3 | 205374 | 1.5E−05 | 0 | 1 | | 1.3E−06 | | |
| gromAD | | X | | | G | A | missense | | normal | 157 | 114 | 111 | 55 | 138 | 2 | 181054 | 1.5E−05 | 0 | 1 | 86883 | 1.5E−06 | | |
| gromAD | | X | | | C | A | missense | | normal | 76 | 33 | 87 | 65 | 76 | 2 | 182527 | 1.5E−05 | 0 | 1 | 87221 | 1.5E−06 | | |
| gromAD | | X | | | C | G | missense | | normal | 85 | 90 | 68 | | 54 | 2 | 182527 | 1.5E−05 | 0 | 1 | 87221 | 1.5E−06 | | |
| gromAD | | X | | | C | A | missense | | normal | 81 | 66 | 68 | | 55 | 2 | 183490 | 1.5E−05 | 0 | 2 | 87325 | 2.8E−06 | | |
| gromAD | | X | | | G | A | missense | | normal | 87 | 75 | 101 | 7 | 96 | 2 | | 1.5E−05 | 0 | 1 | 87345 | 1.5E−06 | | |
| gromAD | | X | | | G | A | missense | | normal | 122 | 130 | 124 | 125 | 110 | 2 | 182705 | 1.5E−05 | 0 | 1 | 87377 | 1.5E−06 | | |
| gromAD | | X | | | T | A | missense | | normal | 11 | 8 | 11 | 10 | 16 | 2 | | 1.5E−05 | 0 | 1 | 87375 | 1.5E−06 | | 1.5E−06 |
| gromAD | | X | | | C | T | missense | | normal | | 156 | 124 | 36 | 137 | 2 | 182781 | 1.5E−05 | 0 | 1 | 87411 | 1.5E−06 | | |
| gromAD | | X | | | A | G | missense | | normal | 127 | 121 | 135 | 115 | 137 | 2 | 182801 | 1.5E−05 | 0 | 1 | 87435 | 1.5E−06 | | |
| gromAD | | X | | | T | C | missense | | normal | 95 | 52 | 66 | 54 | 46 | 2 | 182834 | 1.5E−05 | 0 | 1 | | 1.5E−06 | | |
| gromAD | | X | | | G | C | missense | | normal | 84 | 78 | 114 | 83 | 96 | 2 | 182768 | 1.1E−05 | 0 | 1 | 67486 | 1.5E−05 | | |
| gromAD | | X | | | T | C | missense | | normal | 103 | 100 | 105 | 104 | 101 | 2 | 182988 | 1.1E−05 | 0 | 1 | 67505 | 1.5E−05 | | |
| gromAD | | X | | | G | G | missense | | normal | 72 | 78 | 81 | | 68 | 2 | | 1.1E−05 | 0 | 1 | 67587 | 1.5E−05 | | |
| gromAD | | X | | | A | T | missense | | normal | 83 | | 106 | 60 | 64 | 2 | 183057 | 1.1E−05 | 0 | 1 | 67585 | 1.5E−05 | | |
| gromAD | | X | | | T | C | missense | | normal | 128 | 134 | 148 | 110 | 123 | 2 | 183003 | 1.1E−05 | 0 | 1 | 67581 | 1.5E−05 | | |
| gromAD | | X | | | A | G | missense | | normal | 129 | 135 | 137 | 121 | 123 | 2 | | 1.1E−05 | 0 | 1 | 67735 | 1.5E−05 | | |
| gromAD | | X | | | C | T | missense | | low | 2 | 2 | 3 | 4 | 3 | 2 | 183200 | 1.1E−05 | 0 | 1 | 67786 | 1.5E−05 | 1.5E−05 | |
| gromAD | | X | | | A | G | missense | | normal | 77 | 112 | 114 | 2 | 82 | 2 | 183481 | 1.1E−05 | 0 | 1 | 67825 | 1.5E−05 | | |
| gromAD | | X | | | C | A | missense | | normal | 88 | | 93 | 106 | 88 | 2 | 204168 | 9.8E−06 | 0 | 1 | | 1.3E−05 | | |
| gromAD | | X | | | A | G | missense | | normal | 52 | 43 | 58 | 48 | 58 | 2 | 204886 | 8.8E−06 | 0 | 2 | 75487 | 2.7E−05 | | |
| gromAD | | X | | | C | T | missense | | normal | 83 | 89 | 97 | | 83 | 1 | | 5.9E−06 | 0 | 1 | | 1.8E−05 | | |
| gromAD | | X | | | A | C | missense | | normal | 103 | 32 | 98 | 125 | 163 | 1 | | 5.6E−06 | 0 | 1 | 64489 | 1.6E−05 | | |
| gromAD | | X | | | G | C | missense | | normal | 137 | 109 | 124 | 154 | 163 | 1 | 173519 | 5.6E−06 | 0 | 1 | 65486 | 1.5E−05 | | |
| gromAD | | X | | | C | T | missense | | normal | 89 | 77 | 84 | | 95 | 1 | | 5.6E−06 | 0 | 1 | 65465 | 1.5E−05 | | |
| gromAD | | X | | | C | T | missense | | normal | 125 | 124 | 113 | 89 | 168 | 1 | | 5.5E−06 | 0 | 1 | | 1.5E−05 | | |
| gromAD | | X | | | G | A | missense | | normal | 77 | 80 | 73 | 72 | 82 | 1 | | 5.5E−06 | 0 | 1 | | 1.5E−05 | | |
| gromAD | | X | | | T | A | missense | | low | 107 | 102 | 98 | 117 | 110 | 1 | | 5.5E−06 | 0 | 1 | 68868 | 1.5E−05 | | |
| gromAD | | X | | | C | T | missense | | normal | 106 | 127 | 126 | 85 | 62 | 1 | | 5.5E−06 | 0 | 1 | | 1.5E−05 | | |
| gromAD | | X | | | G | A | missense | | normal | 16 | 16 | 15 | 25 | 15 | 1 | | 5.5E−06 | 0 | 1 | | 1.5E−05 | | 1.5E−05 |
| gromAD | | X | | | A | C | missense | | low | 73 | 81 | 72 | 71 | 67 | 1 | | 5.5E−06 | 0 | 1 | | 1.5E−05 | | |
| gromAD | | X | | — | C | A | missense | | normal | 76 | 66 | 78 | 77 | 62 | 1 | | 5.5E−06 | 0 | 1 | | 1.5E−05 | | |
| gromAD | | X | | | G | A | missense | | low | 126 | 114 | 130 | | 134 | 1 | | 5.5E−06 | 0 | 1 | | 1.5E−05 | | |
| gromAD | | X | | | C | A | missense | | normal | 116 | 107 | 107 | 130 | 130 | 1 | | 5.5E−06 | 0 | 1 | | 1.5E−05 | | |
| gromAD | | X | | | G | C | missense | | normal | 84 | 73 | 78 | 91 | | 1 | | 5.5E−06 | 0 | 1 | | 1.5E−05 | | |
| gromAD | | X | | | C | T | missense | | low | 17 | 17 | 18 | 17 | 15 | 1 | | 5.5E−06 | 0 | 1 | | 1.5E−05 | | 1.5E−05 |
| gromAD | | X | | | CAAG | C | [symbol] | | normal | 0 | 0 | 0 | 0 | 0 | 1 | | 5.5E−06 | 0 | 1 | | 1.5E−05 | 1.5E−05 | |
| gromAD | | X | | | ATAT | A | [symbol] | | normal | 1 | 1 | 1 | 0 | 0 | 1 | | 5.5E−06 | 0 | 1 | | 1.5E−05 | 1.5E−05 | |
| gromAD | | X | | | T | C | missense | | low | 1 | 1 | 1 | 2 | 0 | 1 | | 5.5E−06 | 0 | 1 | | 1.5E−05 | 1.5E−05 | |
| gromAD | | X | | | G | A | missense | | normal | 9 | 8 | 10 | 10 | 0 | 1 | | 5.5E−06 | 0 | 1 | | 1.5E−05 | | 1.5E−05 |
| gromAD | | X | | | C | T | missense | | normal | 11 | 12 | 10 | 12 | 11 | 1 | | 5.5E−06 | 0 | 1 | | 1.5E−05 | | 1.5E−05 |
| gromAD | | X | | | C | A | missense | | normal | 110 | 95 | 35 | 128 | 121 | 1 | | 5.5E−06 | 0 | 1 | | 1.5E−05 | | |
| gromAD | | X | | | A | G | missense | | normal | 28 | 27 | 35 | 26 | 33 | 1 | | 5.5E−06 | 0 | 1 | | 1.5E−05 | | |
| gromAD | | X | | | G | C | missense | | normal | 51 | 45 | 53 | 54 | 51 | 1 | | 5.5E−06 | 0 | 1 | | 1.5E−05 | | |
| gromAD | | X | | | G | A | missense | | normal | 40 | 37 | 37 | 38 | 46 | 1 | | 5.5E−06 | 0 | 1 | | 1.5E−05 | | |
| gromAD | | X | | | G | A | missense | | normal | 87 | 55 | 54 | 77 | 78 | 1 | | 5.5E−06 | 0 | 1 | | 1.5E−05 | | |
| gromAD | | X | | | A | G | missense | | normal | 128 | 104 | 104 | 145 | 154 | 1 | | 5.5E−06 | 0 | 1 | | 1.5E−05 | | |
| gromAD | | X | | | CA | G | indel- | | no | 1 | 1 | 0 | 1 | 0 | 1 | | 5.5E−06 | 0 | 1 | | 1.5E−05 | 1.5E−05 | |
| | | | | | | | frameshift | | | | | | | | | | | | | | | | |
| gromAD | | X | | | G | T | missense | | normal | 21 | 22 | 23 | 18 | 18 | 1 | | 5.5E−06 | 0 | 1 | | 1.5E−05 | | 1.5E−05 |
| gromAD | | X | | | G | T | missense | | normal | 14 | 15 | 18 | 11 | 12 | 1 | | 5.5E−06 | 0 | 1 | | 1.5E−05 | | 1.5E−05 |
| gromAD | | X | | | G | A | missense | | normal | 54 | 49 | 44 | 67 | 55 | 1 | | 5.5E−06 | 0 | 1 | | 1.5E−05 | | |
| gromAD | | X | | | C | A | missense | | normal | 58 | 52 | 57 | 58 | 61 | 1 | | 5.5E−06 | 0 | 1 | | 1.5E−05 | | |
| gromAD | | X | | | G | A | missense | | normal | 56 | 46 | 51 | 59 | 65 | 1 | | 5.5E−06 | 0 | 1 | | 1.5E−05 | | |
| gromAD | | X | | | G | A | missense | | low | 40 | 50 | 35 | 37 | 31 | 1 | | 5.5E−06 | 0 | 1 | | 1.5E−05 | | |
| gromAD | | X | | | G | A | missense | | low | 198 | 182 | 182 | 219 | 211 | 1 | | 5.5E−06 | 0 | 1 | | 1.5E−05 | | |
| gromAD | | X | | | A | G | missense | | normal | 75 | 74 | 79 | 76 | 52 | 1 | | 5.5E−06 | 0 | 1 | | 1.5E−05 | | |
| gromAD | | X | | | C | T | missense | | normal | 77 | 73 | 56 | 88 | 90 | 1 | | 5.5E−06 | 0 | 1 | | 1.5E−05 | | |
| gromAD | | X | | | C | T | missense | | normal | 79 | 81 | 97 | 87 | 61 | 1 | | 5.5E−06 | 0 | 1 | | 1.5E−05 | | |
| gromAD | | X | | | T | G | missense | | normal | 8 | 0 | 33 | 0 | 0 | 1 | | 5.5E−06 | 0 | 1 | | 1.5E−05 | | 1.5E−05 |
| gromAD | | X | | | T | A | missense | | normal | 29 | 82 | 26 | 26 | 31 | 1 | | 5.5E−06 | 0 | 1 | | 1.5E−05 | | |
| | | | | | | | | | | | | | | | | | | | | | | 2.2E−04 | 4.3E−04 |

8.5E−04

[symbol]

indicates data missing or illegible when filed

The Cumulative MAF of Deleterious TLR7 Alleles is <6.5×10⁻⁴

We also investigated the production and function of all 100 remaining non-synonymous TLR7 variants identified in the general population (141,456 individuals in gnomAD v2.1) that had been reported in men or had a general MAF >10⁻⁵ (FIG. 27D and FIG. 31E, 31F). In total, 96 of these variants were missense and three were in-frame small deletions, one of which was not expressed, 17 were weakly expressed, and the others were normally expressed (FIG. 31E, 31F, Table 15), and one variant was a small deletion creating a frameshift found in one man and resulting in an absence of protein production. Seven of the 100 variants were LOF and 15 were hypomorphic (<25% activity) (Table 15). There were thus 24 deleterious TLR7 variants, including the L988S and A288V variants found in four patients with critical COVID-19 pneumonia. Each of these 24 deleterious variants had an individual MAF <1.3×10⁻⁴ in men and their cumulative MAF in men was 6.5×10⁻⁴ (Table 15, Table 16). The cumulative MAF of strictly LOF TLR7 alleles (excluding hypomorphic alleles) in men is about 2.2×10⁻⁴ (Table 15). Overall, we found 11 LOF and two hypomorphic TLR7 alleles in 14 unrelated men with critical COVID-19 pneumonia, whereas deleterious alleles were not found in men with asymptomatic or paucisymptomatic infection. Moreover, deleterious TLR7 alleles in the general population had individual and collective MAF values in men of <1.3×10⁻⁴ and <6.5×10⁻⁴, respectively (FIG. 27E, Table 15). These findings suggest that X-linked recessive (XR) TLR7 deficiency is a genetic etiology of life-threatening COVID-19 pneumonia in men.

TABLE 16
Summary of TLR7 variants.

| SARS- | Critical COVID | | | | | | | | Unknown | | | | | | | |
|---|---|---|---|---|---|---|---|---|---|---|---|---|---|---|---|---|
| COV-2 | In-house cohort | | | | Reported in previous literature | | | | gnomAD database | | | | Total | | | |
| infection | Loss- | Hypo- | Iso- | | Loss- | Hypo- | Iso- | | Loss- | Hypo- | Iso- | | Loss- | Hypo- | Iso- | |
| Source | of- | mor- | mor- | | of- | mor- | mor- | | of- | mor- | mor- | | of- | mor- | mor- | |
| Activity | function | phic | phic | Total | function | phic | phic | Total | function | phic | phic | Total | function | phic | phic | Total |
| Private | 9 | 2 | 3 | 14 | 3 | 0 | 0 | 3 | — | — | — | — | 12 | 2 | 3 | 17 |
| variant | | | | | | | | | | | | | | | | |
| Variant | 1 | 0 | 3 | 4 | 0 | 1 | 4 | 5 | 7 | 15 | 78 | 100 | 9 | 16 | 84 | 109 |
| reported | | | | | | | | | | | | | | | | |
| in | | | | | | | | | | | | | | | | |
| GnomAD | | | | | | | | | | | | | | | | |
| for men | | | | | | | | | | | | | | | | |
| Total | 11 | 2 | 5 | 18 | 3 | 1 | 4 | 8 | 7 | 15 | 78 | 100 | 21 | 18 | 87 | 126 |

TABLE 17
TLR7 deficient patients with severe/critical COVID-19 and hemizygous relative in our
cohort (clinical information, laboratory findings, and immunological findings).

| [symbol] family | [symbol] A | B | C | D | | E | | F | G | | H | I | | J | | K | | L | M | N |
|---|---|---|---|---|---|---|---|---|---|---|---|---|---|---|---|---|---|---|---|---|
| [symbol] | P1 | P2 | P3 | P4 | P4-B | P5 | P5-B | P6 | P7 | P7-B | P8 | P9 | P9-B | P10 | P10-B | P11 | P11-B | P12 | P13 | P14 |
| [symbol] | [symbol] | [symbol] | | | | | | | | | | | | | | | | | | |
| [symbol] | Amor- | Amor- | Amor- | Amor- | Amor- | Amor- | Amor- | Amor- | Amor- | Amor- | Hypo- | Amor- | Amor- | Hypo- | Hypo- | Amor- | Amor- | Amor- | Amor- | Amor- |
| | phic | phic | phic | phic | phic | phic | phic | phic | phic | phic | morphic | phic | phic | morphic | morphic | phic | phic | phic | phic | phic |
| Age | 45 | 60 | 24 | 13 | 5 | 39 | 38 | 55 | 50 | 49 | 19 | 33 | 39 | 43 | 27 | 13 | 5 | 26 | 41 | 71 |
| Sex | Male | Male | Male | Male | Male | Male | Male | Male | Male | Male | Male | Male | Male | Male | Male | Male | Male | Male | Male | Male |
| Ethnic | [symbol] | Euro- | Middle | Middle | Middle | Middle | ND | Euro- | Euro- | Euro- | Euro- | Euro- | Euro- | Latino | Latino | Middle | ND | Middle | ME | Euro- |
| origin | American | pean | East | East | East | East | | pean | pean | pean | pean | pean | pean | | | East | | East | | pean |
| Residence | Spain | France | [symbol] | Turkey | Turkey | Iran | Iran | Italy | Spain | Spain | Italy | Spain | Spain | Colum- | Colum- | France | France | Iran | Turkey | Italy |
| | | | | | | | | | | | | | | bia | bia | | | | | |
| Medical | None | None | None | None | None | [symbol] | None | None | None | None | None | None | None | None | None | None | None | [symbol] | None | None |
| history | | | | | | | | | | | | | | | | | | pneu- | | |
| Covid | | | | | | | | | | | | | | | | | | monia | | |
| detection | | | | | | | | | | | | | | | | | | | | |
| Clinical | Fever, | [symbol] | [symbol] | Cough, | Pneu- | [symbol] | NA | Fever | pneumonia | Fever, | Fever, | Fever, | | Fever, | Fever, | pneu- | NA | nd | [symbol] | fever, |
| presen- | [symbol] | | | fever | monia | | | [symbol] | [symbol] | cough | cough, | cough, | | cough, | cough, | monia, | | | fever ( [symbol] | [symbol] |
| tation | | | | [symbol] | | | | | | | [symbol] | [symbol] | | [symbol] | [symbol] | fever, | | | | |
| (day of | | | | | | | | | | | | | | | | cough, | | | fatigue, [symbol] | |
| admiss- | | | | | | | | | | | | | | | | | | | cough, | |
| ion) | | | | | | | | | | | | | | | | | | | back pain | |
| [symbol] | Yes, | Yes, | Yes, | Yes, | Yes, | Yes, | Yes, | Yes, | Yes, | Yes, | Yes, | Yes, | Yes, | Yes, | Yes, | Yes, | Yes, | Yes, | Yes, | Yes, |
| | Pos- | [symbol] | [symbol] | Pos- | Pos- | ND | Pos- | ND | Pos- | Neg- | Pos- | Pos- | Neg- | Pos- | Pos- | Pos- | Pos- | Pos- | Pos- | Pos- |
| [symbol] | itive | | | itive | itive | | itive | | itive | ative | itive | itive | ative | itive | itive | itive | itive | itive | itive | itive |
| Severely | Critical | Critical | Critical | Critical | Mild | Critical | Aympto- | Critical | Critical | NA | Critical | Critical | NA | Critical | Mod- | Critical | Aympto- | Critical | Critical | Critical |
| classifi- | | | | | | | matic | | | | | | | | erate | | matic | | | |
| cation | | | | | | | | | | | | | | | | | | | | |
| (asympto- | | | | | | | | | | | | | | | | | | | | |
| matic, | | | | | | | | | | | | | | | | | | | | |
| mild, | | | | | | | | | | | | | | | | | | | | |
| moderate, | | | | | | | | | | | | | | | | | | | | |
| severe, | | | | | | | | | | | | | | | | | | | | |
| critical) | | | | | | | | | | | | | | | | | | | | |
| [symbol] | 60 | 10 | 20 | 20 | | 23 | NA | 42 | 10 | NA | 117 | 14 | NA | 13 | [symbol] | [symbol] | NA | [symbol] | 11 | 23 |
| [symbol] | 28 | 9 | 20 | yes | no | 23 | NA | 7 | no | NA | 94 | 10 | NA | 9 | NA | | NA | 3 + 1 | No | None |
| [symbol] | Yes/Yes | Yes/Yes | Yes/Yes | Yes/No | No/No | Yes/Yes | NA | Yes/Yes | Yes/No | NA | Yes/Yes | Yes/Yes | NA | Yes/Yes | Yes/No | Yes/Yes | NA | Yes/No | Yes/No | Yes/No |
| Treatment | [symbol] | [symbol] | [symbol] | [symbol] | None | high dose | NA | [symbol] | [symbol] | NA | [symbol] | [symbol] | NA | [symbol] | | [symbol] | NA | [symbol] | [symbol] | [symbol] |
| | | | | | | [symbol] | | | | | | | | | | | | | | |
| Medical | | NA | ND | None | NA | Several | NA | ND | None | NA | None | NA | None | None | None | NA | None | None | None | None |
| [symbol] | | | | | | | | | | | | | | | | | | | | |
| Biological | | | | | | | | | | | | | | | | | | | | |
| parameters | | | | | | | | | | | | | | | | | | | | |
| Hemo- | 14.4 | 13.4 | | 14.5 | 12.8 | | | | 15.3 | 15.3 | 16.2 | 17.4 | | 18.50 | | | | | 14 | 15.5 |
| globin | | | | | | | | | | | | | | | | | | | | |
| [symbol] | | | | | | | | | | | | | | | | | | | | |
| [symbol] | 137 | 8.6 | | 353 | 316 | | | | 179 | | 184 | 190 | | 119 | | 243 | | | 199 | 309 |
| [symbol] | 5.8 | 13.5 | | | | | | | | | | | | | | | | | | |
| [symbol] | 300 | 1.2 | | | | | | | 2100 | 2100 | | | | | | | | | 9.81 | 12.87 |
| 3 [symbol] | | | | | | | | | | | | | | | | | | | | |
| [symbol] | | | | | | | | | | | | | | | | | | | | |
| T cells | | | | | | | | | | | | | | | | | | | | |
| [symbol] | | | | | | | | | | | | | | | | | | | | |
| T cells | | | | | | | | | | | | | | | | | | | | |
| [symbol] | | | | | | | | | | | | | | | | | | | | |
| [symbol] | | 420 | | | 390 | | | | 500 | 500 | | | | | | | | | | |
| [symbol] | | | | | | | | | | | | | | | | | | | | |
| Antibodies | | | | | | | | | | | | | | | | | | | | |
| [symbol] | | | 1.8 | 0.9 | | | | | | | | | | | | | | | | |
| [symbol] | | | 8.8 | 9 | | | | | | | | | | | | | | | | |
| [symbol] | | | 2.2 | 1.5 | | | | | | | | | | | | | | | | |
| [symbol] | | | 20 | 20 | | | | | | | | | | | | | | | | |
| Type IFNs | Neg- | ND | ND | Neg- | Neg- | ND | Neg- | ND | neg- | neg- | Neg- | Neg- | | | | Yes | neg- | Neg- | ND | Neg- |
| [symbol] | ative | | | ative | ative | | ative | | ative | ative | ative | ative | | | | but not | ative | ative | | ative |
| [symbol] | | | | | | | | | | | | | | | | [symbol] | | | | |
| Comor- | No | | ND | Obesity | No | ND | ND | ND | arterial | arterial | ND | None | ND | None | None | ND | ND | ND | No | hyper- |
| bidity | | | | | | | | | hyper- | hyper- | | | | | | | | | | tension, |
| [symbol] | | | | | | | | | tension | tension | | | | | | | | | | obesity |
| CRP | 34 | 157 | 46 | | | | | | | | | | 102 | | 519 | | 136 | | 56.2 | 20.3 |
| (mg/ [symbol] ) | | | | | | | | | | | | | | | | | | | | |

[symbol]

indicates data missing or illegible when filed

Incomplete Clinical Penetrance of Inherited TLR7 Deficiency

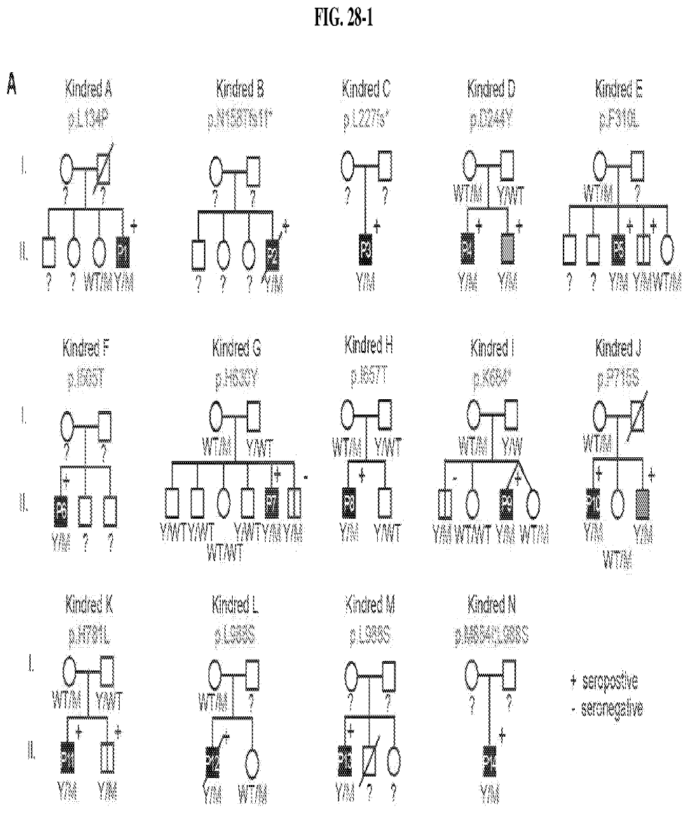

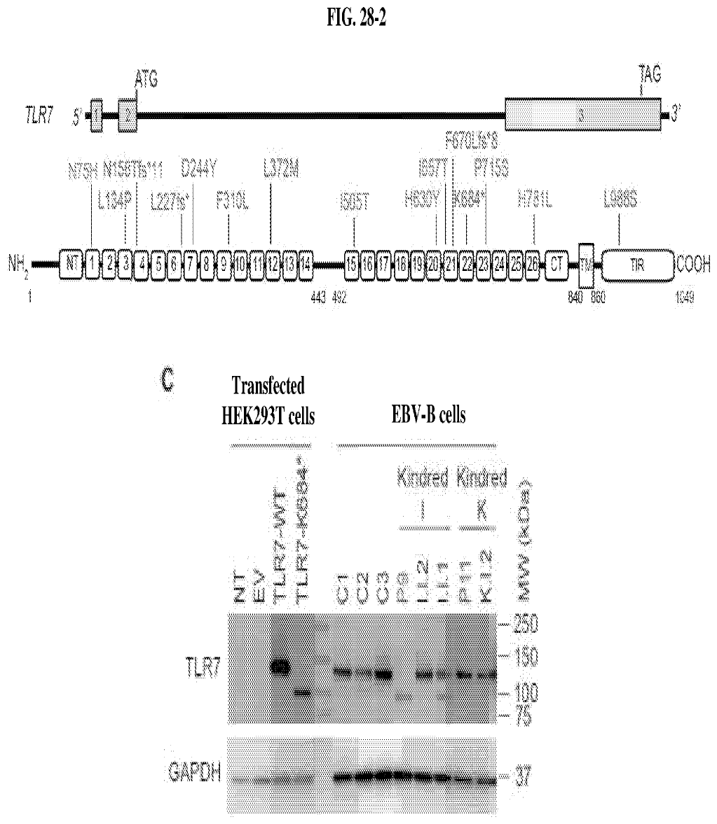

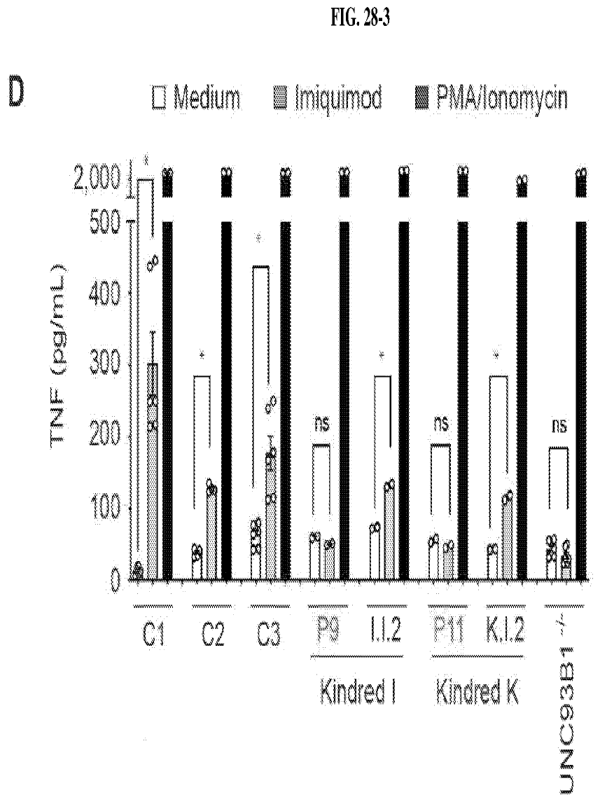

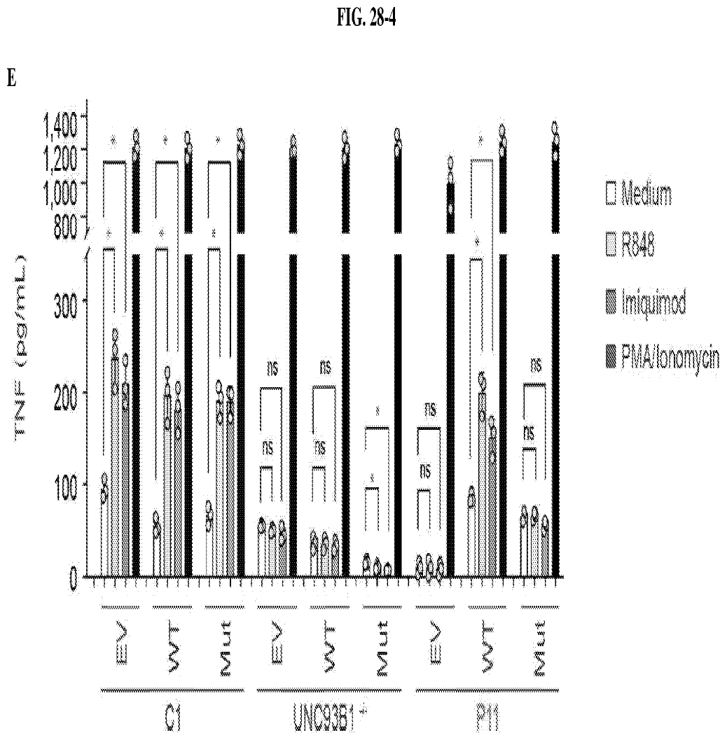

The 14 patients were of three major ethnic origins, as confirmed by principal component analysis (PCA) of their exomes or genomes (34), and they were resident in six countries (France n=2, Spain n=3, Italy n=3, Turkey n=2, Iran n=3, Colombia n=1) (FIG. 28A, FIG. 28B, FIG. 31, Table 11, Table 17). The patients were hospitalized for critical COVID-19 between March 2020 and May 2021. Blood samples (diluted 1/10) from these 14 patients did not carry auto-Abs neutralizing 10 ng/mL (9) or even 100 pg/mL IFN-α2 or -ω (Bastard P.; Examples 2 and 4). They were aged 13 to 71 years and their mean age was lower than that of the total cohort (mean age of 36.7 years, versus 52.9 years for the total cohort, in which age ranged from 0.5 to 99 years). TLR7-deficient patients accounted for about 1.6% of the patients below the age of 70 years (13 patients) and 1.4% of the entire cohort (14 patients). Two patients died and 12 survived (FIG. 28A—Table 11). None had previously been hospitalized for a severe viral illness, including influenza pneumonia. Sanger sequencing of the TLR7 locus in the relatives of these patients identified the deleterious alleles in 13 heterozygous women from nine families and six hemizygous men from six families (FIG. 28A). None of the TLR7 variants were de novo in the index cases. Four of the six hemizygous relatives of the index cases had antibodies against SARS-CoV-2 (FIG. 28A, Table 17). One was a 38-year-old adult with no relevant clinical history, another was a 27-year-old adult hospitalized for moderate pneumonia, and the remaining two were five-year-old boys, one of whom had been hospitalized for moderate COVID-19 pneumonia, the other having no relevant clinical history (Table 17). The clinical penetrance of critical COVID-19 in men is therefore high, but not complete, particularly in young patients. Blood pDC counts decrease with age (35-37), and this may contribute to the apparent increase in penetrance with age.

Deleterious TLR7 Alleles Abolish B-Cell Responses to TLR7 Agonists

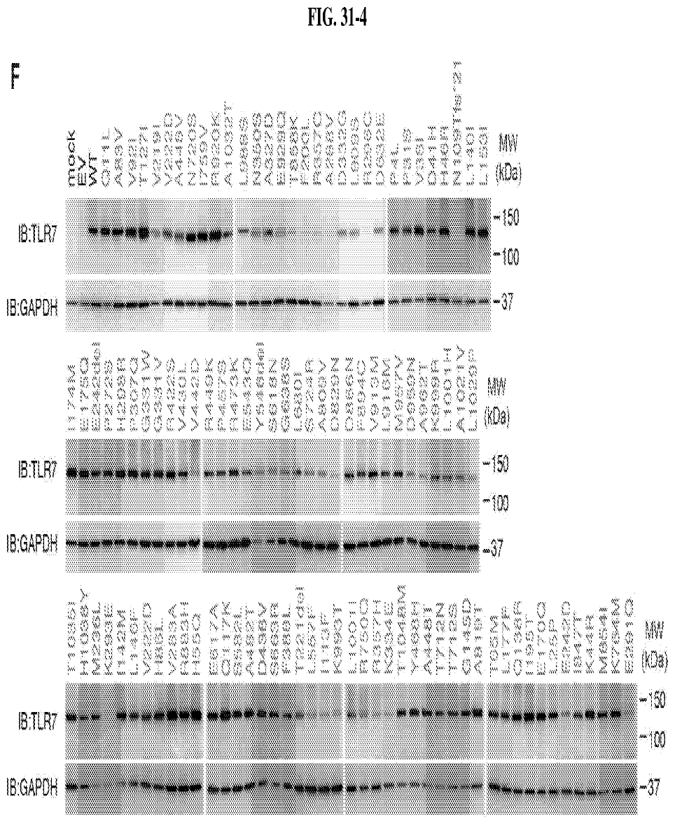

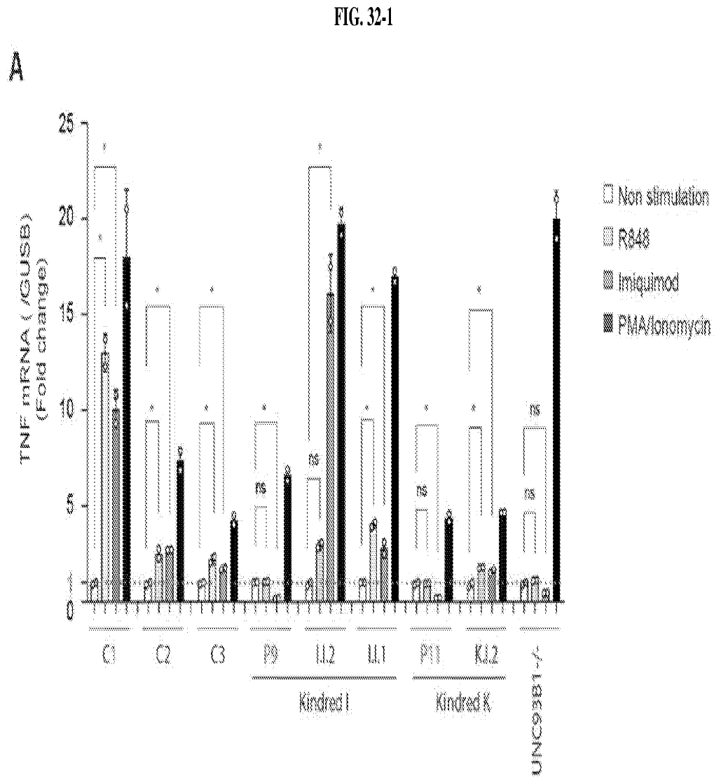

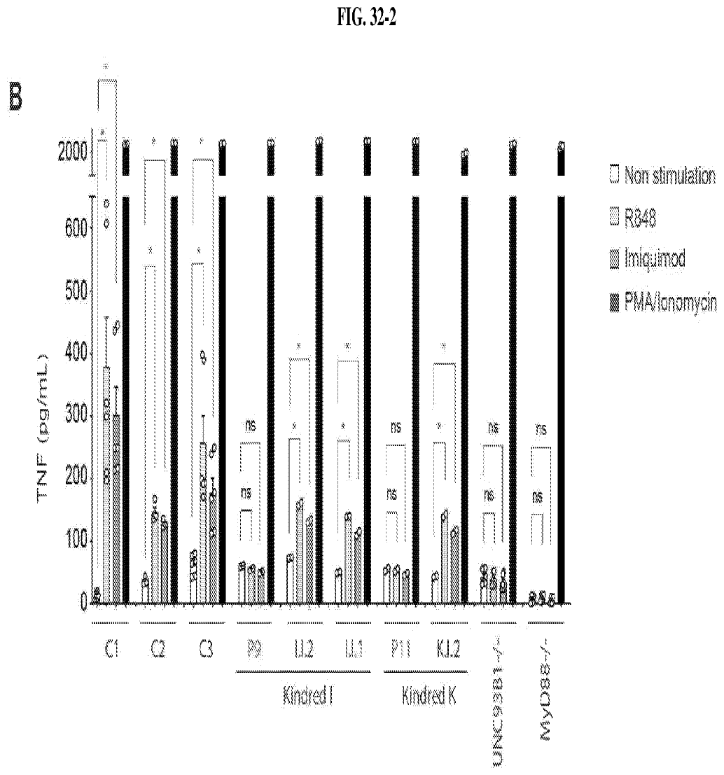

As a first approach to testing the impact of deleterious TLR7 alleles in the patients' cells, we tested Epstein-Barr virus-transformed B-cell lines (EBV-B cells) from healthy controls and patients carrying the hemizygous p.K684* (P9) or p.H781L (P11) variants. The endogenous expression of the p.H781L TLR7 protein was normal, whereas p.K684* generated a truncated protein (FIG. 28C). In response to agonists of TLR7 (imiquimod) or TLR7 plus TLR8 (R848), the EBV-B-cell lines carrying these two mutations failed to produce TNF (FIG. 28D, FIG. 32A, 32B). The lentiviral transduction of these TLR7-deficient EBV-B cells with a WT TLR7 cDNA was unsuccessful, despite numerous attempts, and this was also the case for control EBV-B cells, perhaps because the overproduction of TLR7 is toxic in B cells (38). Consistent this view, we were able to express this cDNA in IRAK4- or MyD88-deficient EBV-B cells. We therefore investigated whether the addition of an IRAK4 inhibitor (PF06650833) would permit the expression of WT TLR7 in control and TLR7-mutated EBV-B cells. This approach was successful, and WT TLR7 expression restored responses to TLR7 agonists (after removal of the inhibitor) (FIG. 28E). Hemizygosity for LOF TLR7 alleles thus abolished responses to TLR7 stimulation in EBV-B cells, a phenotype that was rescued by WT TLR7 expression. Collectively, these findings further suggest that XR TLR7 deficiency is a genetic etiology of critical COVID-19 pneumonia.

The Patients' Myeloid Cells, Including pDCs, do not Respond to TLR7 Agonists

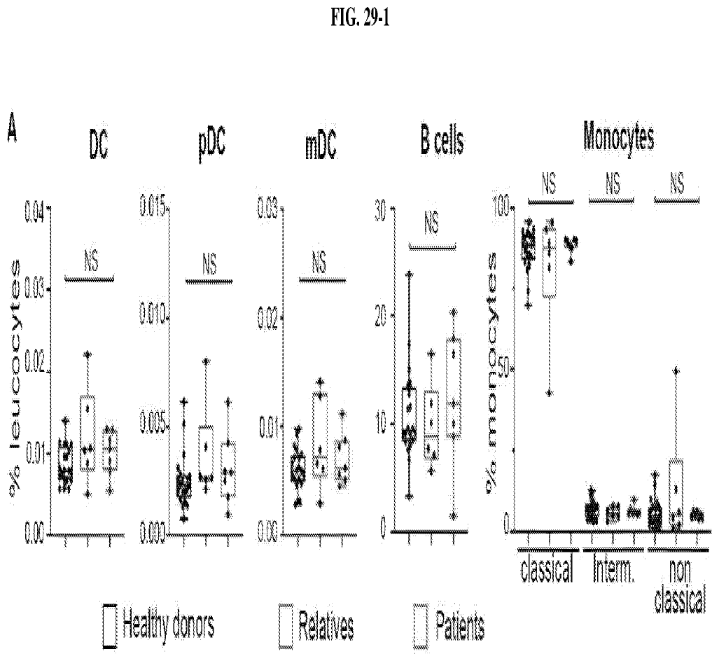

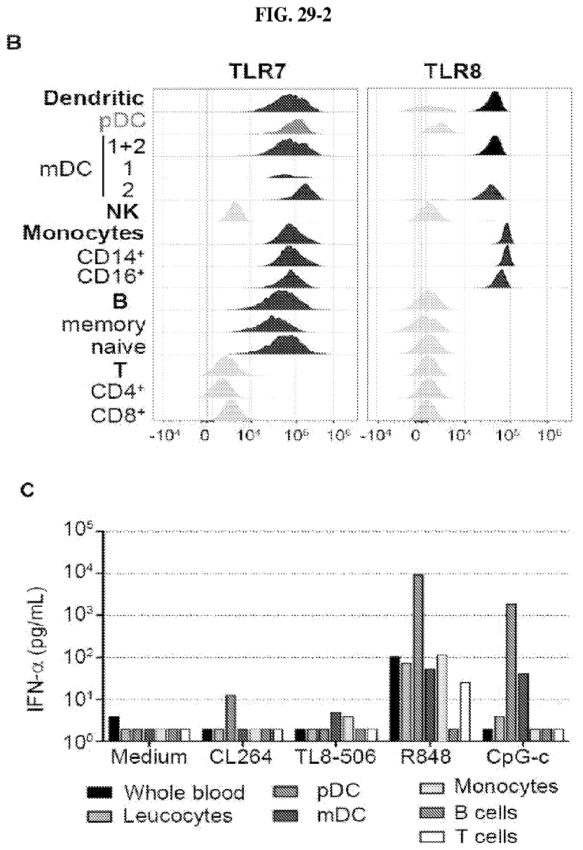

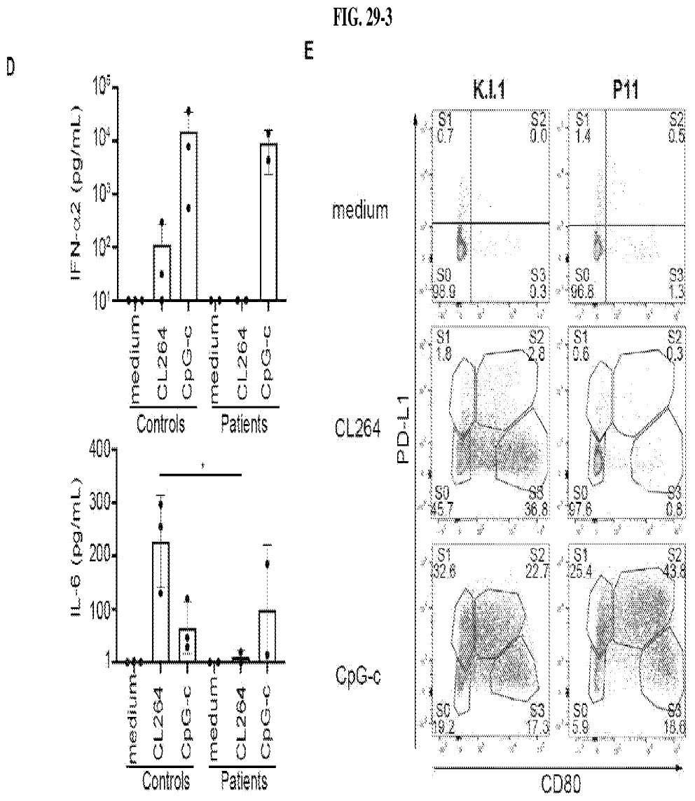

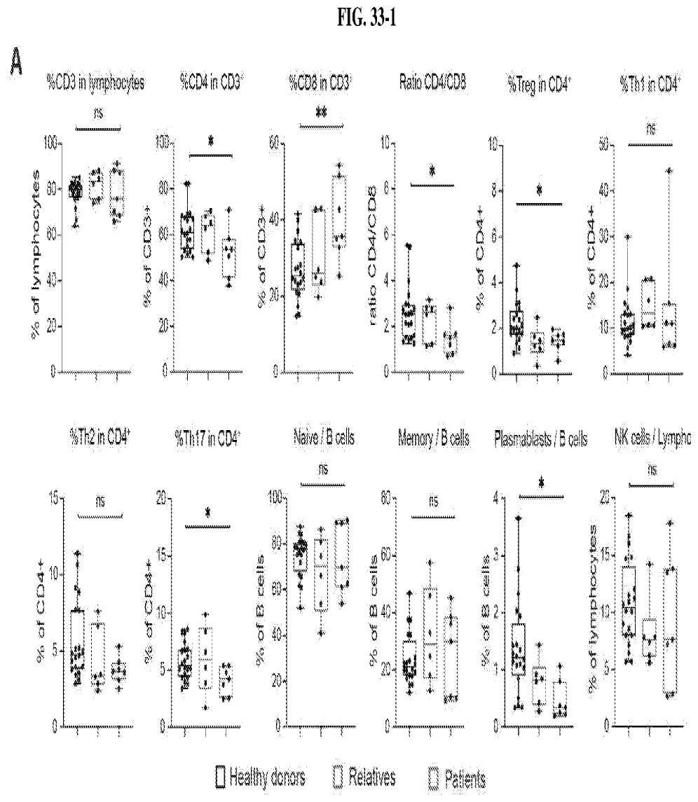

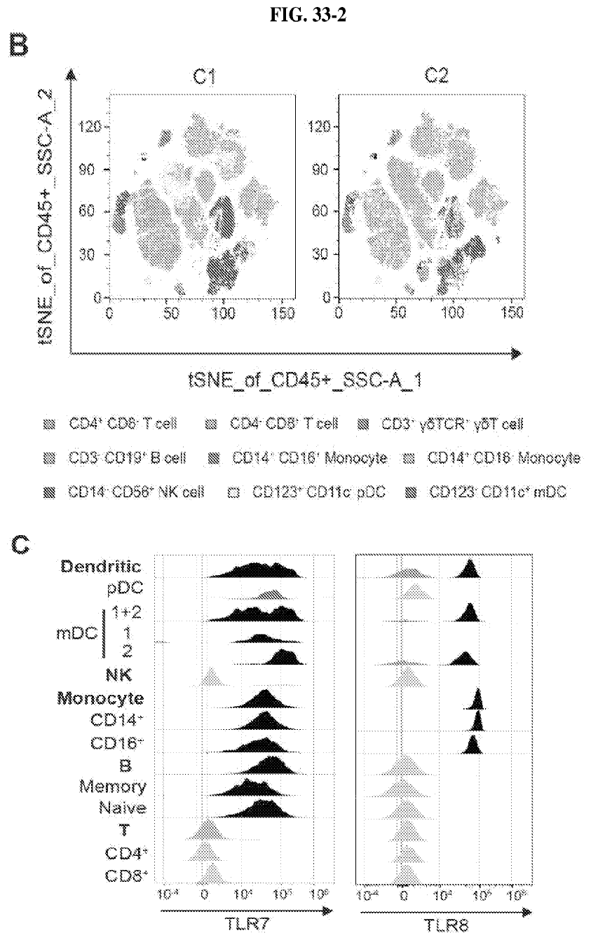

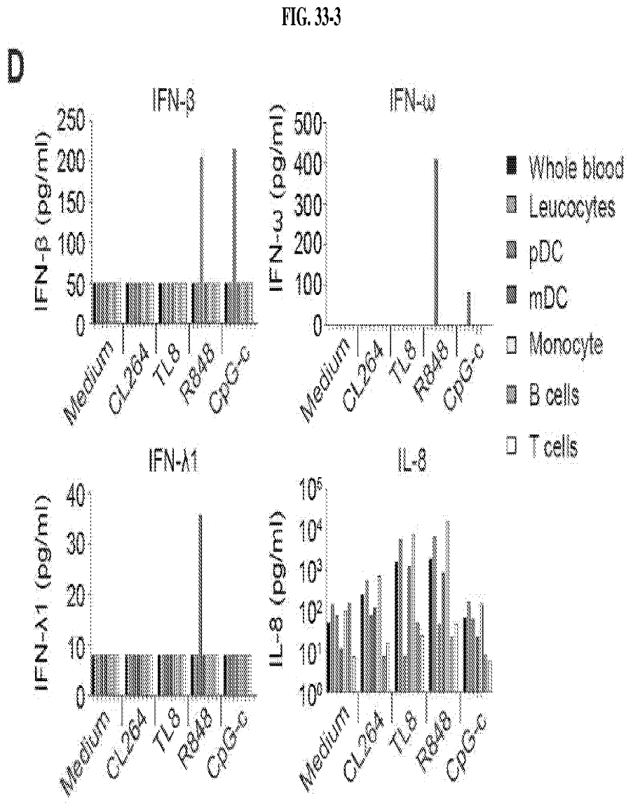

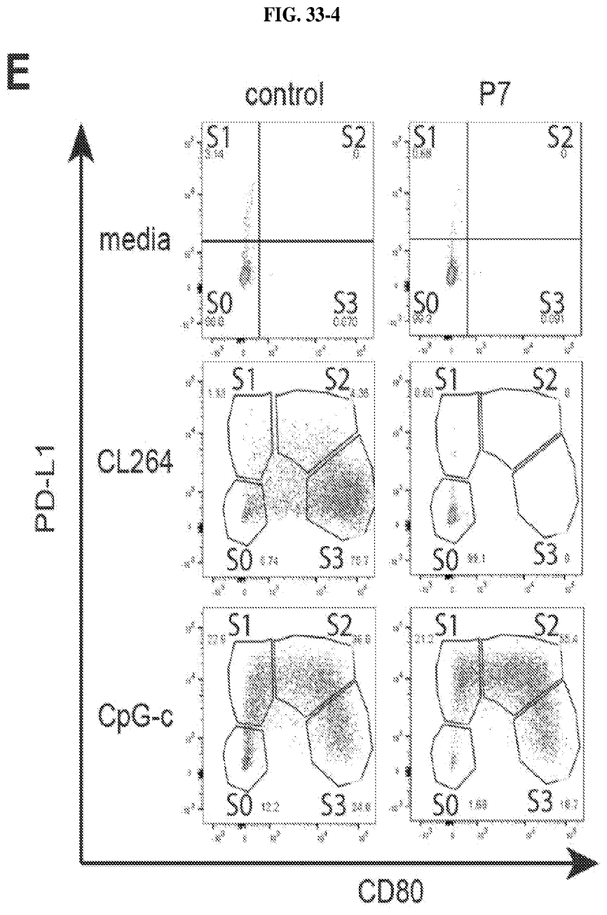

Human TLR7 is only known to be expressed and functional in leukocyte subsets: plasmacytoid and classical dendritic cells (pDCs and rmDCs), monocytes (classical, intermediate, and non-classical), and B cells (26, 31, 39). TLR8 is expressed in mDCs but not pDCs, monocytes but not B cells, and neutrophils (unlike TLR7) (26, 31, 39). Neither TLR7 nor TLR8 mRNAs have been detected in the lung or pulmonary epithelial cells (40). Deep immunophenotyping by CyTOF in seven patients with TLR7 deficiency revealed no major abnormalities in 18 leukocyte subsets, including pDCs, mDCs, monocytes, and B cells (FIG. 29A, FIG. 33A). We previously reported inherited IRF7 deficiency in a child with critical influenza pneumonia (5) and two unrelated adults with critical COVID-19 pneumonia (8). This defect disrupts the amplification of type I IFNs in all cell types, including pDCs, which are normally the main producers of type I IFN upon blood cell stimulation with TLR7 agonists or viruses, due to their constitutive expression of IRF7 (26, 41-43). We hypothesized that TLR7 deficiency in pDCs impairs the production of type I IFN by these cells in response to ssRNA. We confirmed that TLR7 was expressed on pDCs, and that TLR8 was not (FIG. 29B, 33B, 33C). We measured the production of type I IFNs by purified leukocyte subsets (pDCs, mDCs, monocytes, B cells, T cells), in response to TLR7, TLR8 and TLR9 agonists (FIG. 29C, FIG. 33D). We confirmed that pDCs produced 100-1,000 times more type I IFN per cell than other leukocyte subsets upon TLR7 stimulation (FIG. 29C, FIG. 33D). We purified pDCs from P7 and P11 and analyzed their production of type I IFNs in response to CL264 and class C CpG oligonucleotide (CpG-c), relative to that of pDCs from healthy relatives, using a cytometric bead array (CBA) (FIG. 29D). pDCs from P7 and P11 did not produce type I IFNs upon stimulation with a TLR7 agonist, whereas they responded to a TLR9 agonist (FIG. 29D, 29E). Moreover, agonist-induced upregulation of PD-L1 and CD80 defines the maturation of pDCs into the S1 (PD-L1^{high}/CD80^{low}), S2 (PD-L1^{high}/CD80^{high}), and S3 (PD-L1^{low}/CD80^{high}) subsets (44). This maturation was not observed in the pDCs of P7 and P11, but was detected in the pDCs of heathy relatives and controls (FIG. 29E, FIG. 33E). Thus, pDCs from patients with TLR7 mutations do not respond to TLR7 agonists in terms of maturation into specialized subsets and type I IFN production.

The Patients' pDCs Respond Poorly to SARS-CoV-2

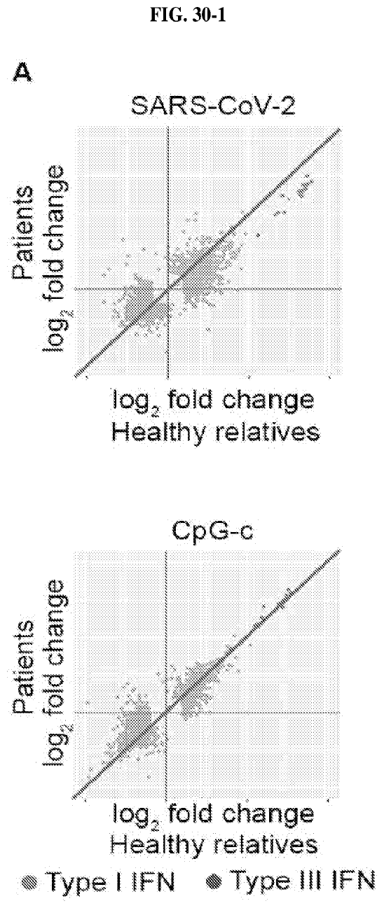

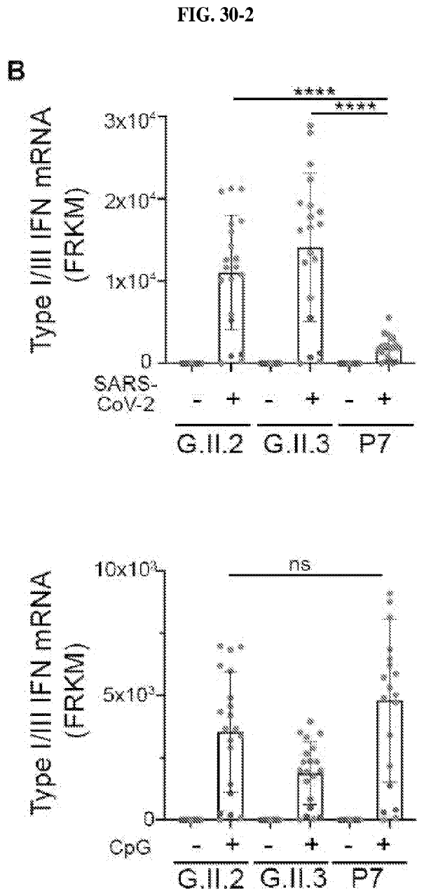

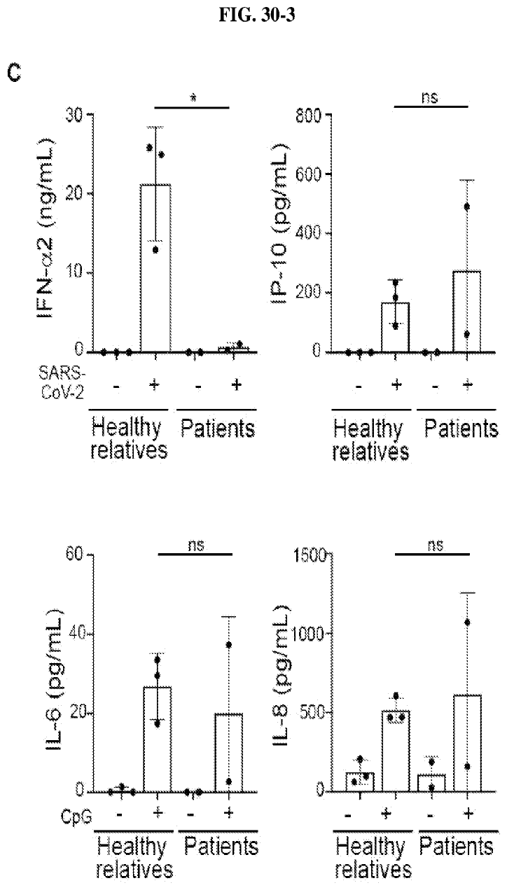

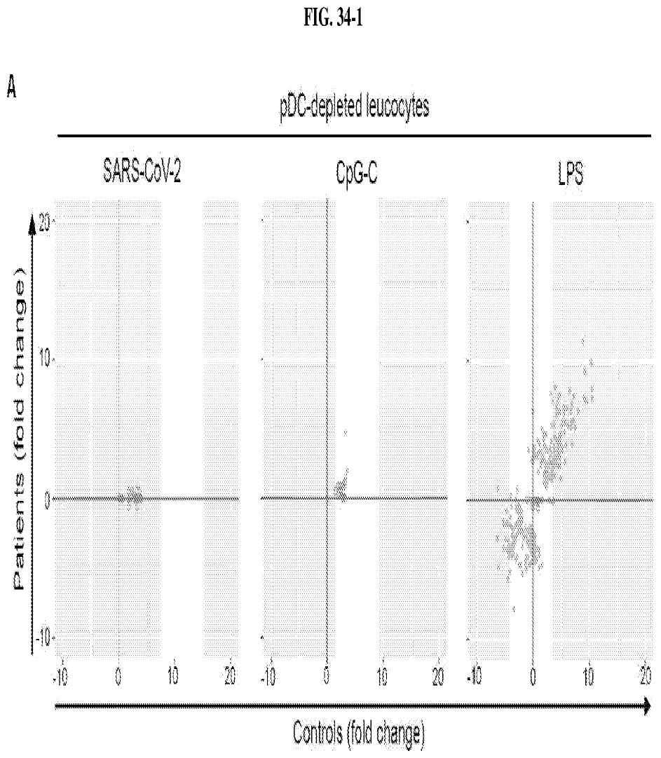

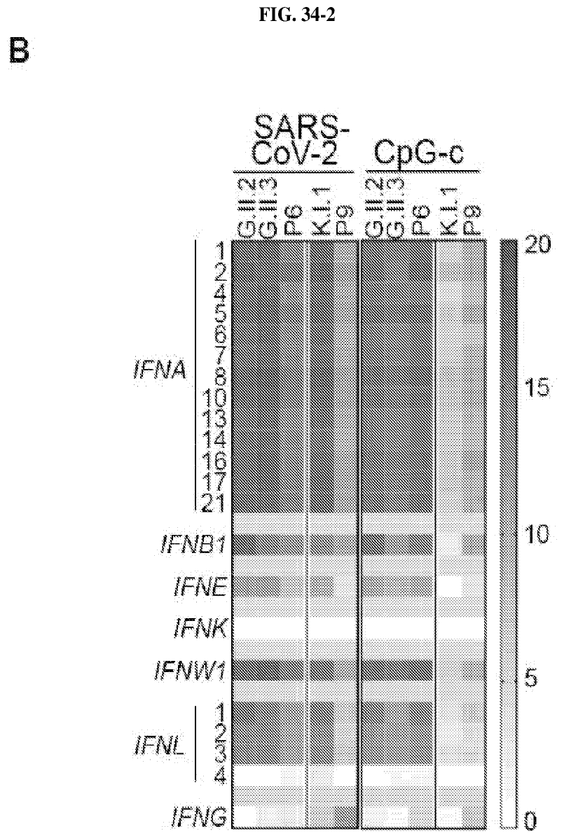

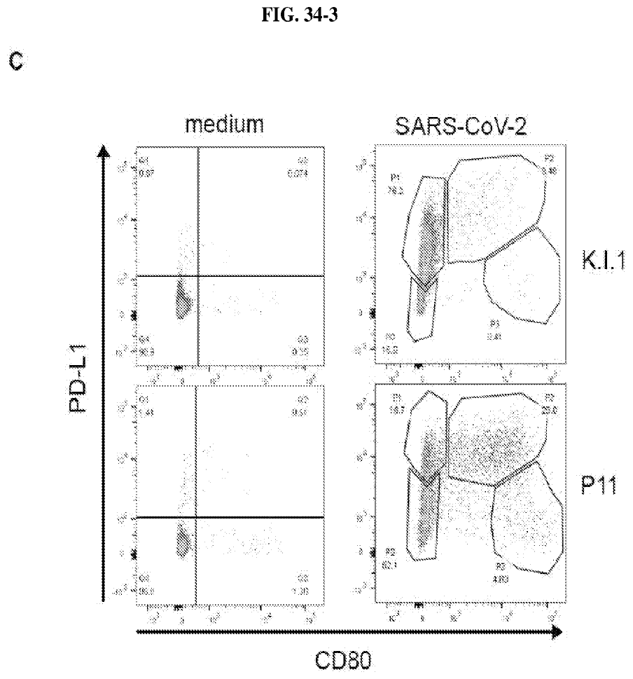

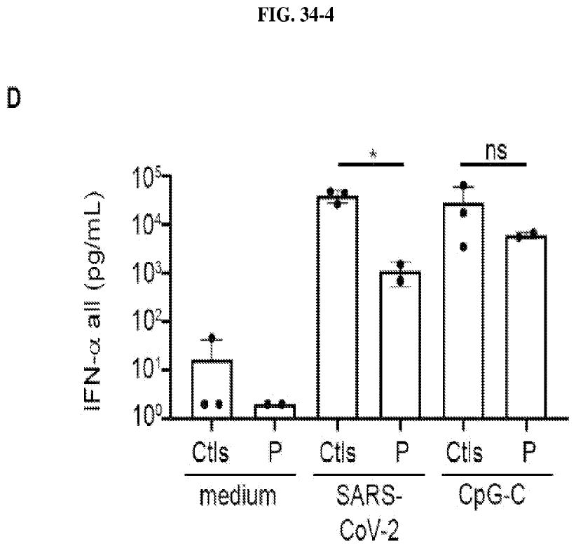

A plausible mechanism accounting for the severity of COVID-19 in TLR7-deficient patients is the impairment of type I IFN production by pDCs upon stimulation with SARS-CoV-2, which can enter these cells, but cannot replicate productively within them (44, 45). Indeed, we previously showed that the activation of human pDCs by SARS-CoV-2 depends on IRAK4 and UNC-93B, but not TLR3 (44). We tested the hypothesis that TLR7 is an essential pDC sensor of SARS-CoV-2, upstream from IRAK4 and UNC-93B, by infecting pDCs and pDC-depleted leukocytes from healthy controls and TLR7-deficient patients with SARS-CoV-2 for 24 hours. Control pDC-depleted leukocytes infected with SARS-CoV-2 displayed no significant up- or downregulation of gene expression (FIG. 34A). By contrast, transcriptomic analysis showed a strong upregulation of the type I IFN transcriptional module in pDCs from healthy controls, which was greatly reduced in pDCs from TLR7-deficient patients (FIG. 30A). Induction of the 17 type I IFN genes in pDCs from TLR7-deficient patients was 10 to 100 times weaker than that in pDCs from healthy individuals (FIG. 30B, 34B). We also analyzed the functional specialization of pDC subsets (S1-, S2-, and S3-pDC subsets) in response to SARS-CoV-2 activation (44, 46). pDCs from P11 cultured with SARS-CoV-2 for 24 hours displayed abnormally low levels of maturation into the S1-subset—the pDC subset principally responsible for IFN-α production upon SARS-CoV-2 infection (FIG. 34C). Finally, we evaluated the amount of type I IFN secreted by SARS-CoV-2-infected pDCs. All 13 individual IFN-06 forms were produced in significantly smaller amounts by TLR7-deficient pDCs than by control pDCs (FIG. 30C, 34D). However, IFN-α production by TLR7-deficient pDCs upon SARS-CoV-2 was impaired, but not entirely abolished, as in UNC93B- or IRF7-deficient pDCs (8, 44), implying that there are also TLR7-independent sensors of SARS-CoV-2 in pDCs. Thus, SARS-CoV-2 triggers type I IFN induction in pDCs in a manner that is dependent on TLR7, but not exclusively so. As pDCs are normally the main leukocytes producing type I IFN in such conditions, and type I IFN is essential for protective immunity to SARS-CoV-2 (8, 9), these findings suggest that XR TLR7 deficiency underlies critical COVID-19 pneumonia by disrupting TLR7- and pDC-dependent type I IFN production.

Concluding Remarks

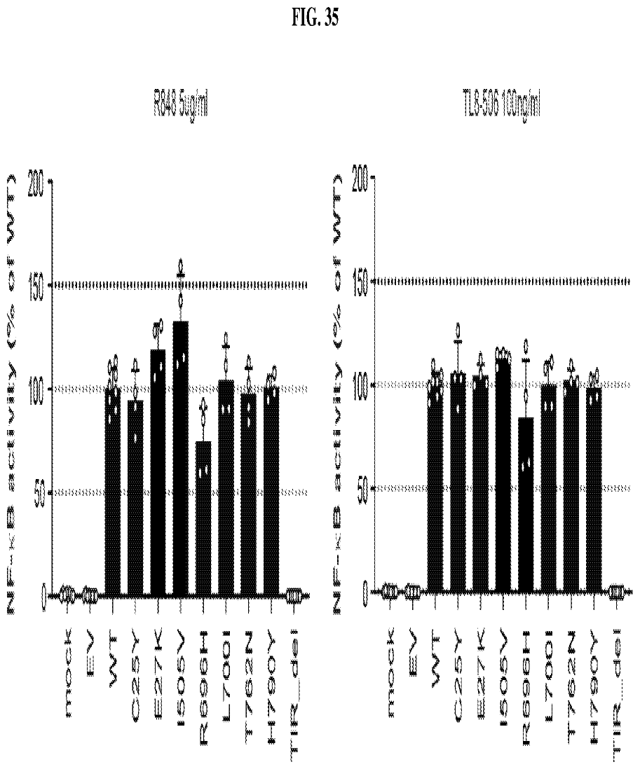

We report XR TLR7 deficiency as a genetic etiology of critical COVID-19 pneumonia in 14 unrelated male patients, aged 13 to 71 years, from six countries. Only one of these 14 patients (7%) was older than 70 years, consistent with our previous observation that only two of 23 patients (8.7%) with inborn errors of TLR3-dependent type I IFN immunity were older than 65 years (8). This suggests that these genetic defects are mostly found in the youngest patients. This contrasts with the auto-Abs to type I IFNs, which are found mostly in patients older than 70 years, and not in patients with inborn errors of TLR3- or TLR7-dependent type I IFN ((8, 9); Bastard P. et al. accompanying report, Examples 2 and 4). TLR7-deficient patients accounted for about 1.6% of the male patients with critical COVID-19 pneumonia below the age of 70 years in our cohort. This discovery provides a first explanation for the higher risk of critical disease in men than in women under the age of 70 years, complementing our previous observation of a much higher frequency of neutralizing auto-Abs against type I IFNs in men than in women with critical COVID-19 pneumonia for patients over the age of 65 years (9). Previously reports of patients with critical COVID-19 pneumonia due to inborn errors of TLR3-dependent type I IFN immunity (8), including autosomal recessive IRF7 or IFNAR1 deficiency (5, 6), or due to auto-Abs neutralizing type I IFNs (9, 11-14, 16, 17), strongly suggest that critical disease in TLR7-deficient patients is a consequence of impaired type I IFN production upon SARS-CoV-2 infection. The absence of biochemically deleterious X-linked TLR8 variants in our cohort of patients (FIG. 35) suggests that TLR8 is not essential for host defense against SARS-CoV-2. This is consistent with the modest capacity of TLR8 to induce type I IFN and its lack of expression on pDCs (26), and with the inflammatory phenotype of TLR8 gain-of-function mutations, which do not underlie a type I interferonopathy (28-30). Patients with inherited IRAK4 or MyD88 deficiency, whose cells do not respond to the stimulation of IL-1Rs and TLRs other than TLR3, including TLR7, are predicted to be vulnerable to SARS-CoV-2 (47, 48). However, they have not been reported to display any severe viral illness over the almost 20 years since the discovery of IRAK-4 deficiency (49-51). This is intriguing, as strong negative selection operates at the human TLR7, TLR8, and TLR9 loci (49, 52). Our study provides a first answer to this riddle, by establishing that TLR7 is essential for protective immunity to SARS-CoV-2. As critical COVID-19 and seasonal influenza can be caused by inborn errors of TLR3-dependent type I IFN immunity (5-8), it is tempting to speculate that TLR7 might also be essential for host defense against more virulent, pandemic influenza viruses.

Through the discovery of the essential nature of TLR7 for the induction of type I IFN in response to SARS-CoV-2, our study also reveals the essential function of human pDCs in host defense. The constitutively high levels of IRF7 expression of these cells make them the most potent producers of type I IFN in the blood, and perhaps in the entire human body, and this has long suggested a possible key role in antiviral immunity (24). However, the essential and redundant roles of this particular leukocyte subset have yet to be determined, in the absence of human pDC-specific deficiencies causally underlying a clinical phenotype. It has long been suspected, but never proved, that pDCs are essential for host defense (25, 53-55). Inherited IRF7 deficiency, which underlies critical influenza or COVID-19 pneumonia, disrupts the production of type I IFNs not only by pDCs (5, 8), but also by all other cell types, not only in the blood, but also in other tissues, including fibroblasts and pulmonary epithelial cells (5). Likewise, patients with GATA2 deficiency, who are prone to critical influenza (56), lack pDCs, but these patients also lack many other blood myeloid and lymphoid cell subsets, including monocytes, NK, and B cells (57-60). It therefore remains unclear whether IRF7 and GATA2 deficiencies confer a predisposition to critical viral pneumonia via their impact on pDCs or through effects on other leukocytes or pulmonary cells. Our study clarifies this matter, as TLR7 is expressed by only a few leukocyte subsets, including pDCs, which are the only potent type I IFN producers among these subsets, and TLR7 is not expressed by pulmonary epithelial cells. The expression of both TLR7 and IRF7 is a feature unique to pDCs (61, 62). While inborn errors of TLR3 underlie critical COVID-19 pneumonia by impairing the production of type I IFNs by cells other than pDCs, such as pulmonary epithelial cells (5-8, 63), inborn errors of TLR7 are pathogenic by impairing the production of type I IFNs by pDCs. Inborn errors of IRF7 and IFNAR1 have a broader impact, on both pulmonary cells and pDCs (5, 6, 8). pDCs express other viral sensors, including TLR9 (for DNA), MDA5 and RIG-I (for dsRNA) (64), but TLR7 deficiency impairs their capacity to respond to SARS-CoV-2 by producing sufficient quantities of type I IFNs. Our findings therefore indicate that both human TLR7 and pDCs are essential for type I IFN-dependent protective immunity to SARS-CoV-2.

Methods

Cohort Recruitment and Consent

This study included 1,005 male patients with life-threatening COVID-19 pneumonia, defined as patients with pneumonia who developed critical disease, whether pulmonary with high-flow oxygen or mechanical ventilation (CPAP, BIPAP, intubation), septic shock, or any other type of organ damage requiring ICU admission. Patients who developed Kawasaki-like syndrome were excluded. The age of the patients ranged from 0.5-99 years, with a mean age of 52.9 years (SD 15.6 years). Asymptomatic or paucisymptomatic individuals were recruited on the basis of positive PCR or serological tests for SARS-CoV-2 in the absence of symptoms. These individuals were close contacts of patients or were recruited after clinical screening. The age of the asymptomatic or paucisymptomatic individuals ranged from 1.3-102 years, with a mean age of 41.1 years (SD: 16.1 years).

All the enrolled subjects provided written informed consent and were collected through protocols conforming to local ethics requirements. For patients enrolled in the French COVID cohort (clinicaltrials.gov NCT04262921), ethics approval was obtained from the CPP IDF VI (ID RCB: 2020-A00256-33) or the Ethics Committee of Erasme Hospital (P2020/203). For subjects enrolled in the COV-Contact study (clinicaltrials.gov NCT04259892), ethics approval was obtained from the CPP IDF VI (ID RCB: 2020-A00280-39). For patients enrolled in the Italian cohort, ethics approval was obtained from the University of Milano-Bicocca School of Medicine, San Gerardo Hospital, Monza—Ethics Committee of the National Institute of Infectious Diseases Lazzaro Spallanzani (84/2020) (Italy), and the Comitato Etico Provinciale (NP 4000—Studio CORONAlab). STORM-Health care workers were enrolled in the STudio OsseRvazionale sullo screening dei lavoratori ospedalieri per COVID-19 (STORM-HCW) study, with approval from the local IRB obtained on Jun. 18, 2020. Patients and relatives from San Raffaele Hospital (Milan) were enrolled in protocols COVID-BioB/Gene-COVID and, for additional studies, TIGET-06, which were approved by local ethical committee. For patients enrolled in Spain, the study was approved by the Committee for Ethical Research of the Infanta Leonor University Hospital, code 008-20, Committee for Ethical Research of the University Hospital 12 de Octubre, code 16/368 and the Bellvitge University Hospital code PR127/20, the University Hospital of Gran Canaria Dr. Negrin code 2020-200-1 COVID-19 and the Vall d'Hebron University Hospital, code PR(AMI)388/2016. Anonymized samples were sequenced at the NIAID through USUHS/TAGC under non-human subject research conditions; no additional IRB consent was required at the NIH.

Next-Generation Sequencing

Genomic DNA was extracted from whole blood. For the 1,331 patients included, the whole exome (n=934) or whole genome (n=397) was sequenced at several sequencing centers, including the Genomics Core Facility of the Imagine Institute (Paris, France), the Yale Center for Genome Analysis (USA), the New-York Genome Center (NY, USA), and the American Genome Center (TAGC, USUHS, Bethesda, USA), and the Genomics Division-ITER of the Canarian Health System sequencing hub (Canary Islands, Spain).

For WES, libraries were generated with the Twist Bioscience kit (Twist Human Core Exome Kit), the xGen Exome Research Panel from Integrated DNA Technologies (IDT xGen), the Agilent SureSelect V7 kit or the SeqCap EZ MedExome kit from Roche, and the Nextera Flex for Enrichment-Exome kit (Illumina). Massively parallel sequencing was performed on a HiSeq4000 or NovaSeq6000 system (Illumina). For WES analysis performed at CNAG Barcelona, Spain, capture was performed with the SeqCap EZ Human Exome Kit v3.0 (Roche Nimblegen, USA) and 100-bp paired-end read sequences were obtained on a HiSeq 2000-4000 platform (Illumina, Inc. USA). For the OSR Italian cohort, WES was performed with the Agilent SureSelect V7 kit on a NovaSeq6000 system (Illumina).

For WGS on patients of the Italian cohort (TAGC), genomic DNA samples were dispensed into the wells of a Covaris 96 microTUBE plate (1,000 ng per well) and sheared with the Covaris LE220 Focused-ultrasonicator, at settings targeting a peak size of 410 bp (t:78; Duty:18; PIP:450; 200 cycles). Sequencing libraries were generated from fragmented DNA with the Illumina TruSeq DNA PCR-Free HT Library Preparation Kit, with minor modifications for automation (Hamilton STAR Liquid Handling System), with IDT for Illumina TruSeq DNA UD Index (96 indices, 96 samples) adapters. Library size distribution was assessed and the absence of free adapters or adapter dimers was checked by automated capillary gel electrophoresis (Advanced Analytical Fragment Analyzer). Library concentration was determined by qPCR with the KAPA qPCR Quantification Kit (Roche Light Cycler 480 Instrument II). Sequencing libraries were normalized and combined as 24-plex pools and quantified as above, before dilution to 2.9 nM and sequencing on an Illumina NovaSeq 6000 with the S4 Reagent Kit (300 cycles) and 151+8+8+151 cycle run parameters. Primary sequencing data were demultiplexed with the Illumina HAS2.2 pipeline and sample-level quality control was performed for base quality, coverage, duplicates and contamination (FREEMIX <0.05 by VerifyBamID).

We used the Genome Analysis Software Kit (GATK) (version 3.4-46 or 4) best-practice pipeline to analyze our WES data (65). We aligned the reads obtained with the human reference genome (hg19), using the maximum exact matches algorithm in the Burrows-Wheeler Aligner (BWA) (66). PCR duplicates were removed with Picard tools (picard.sourceforge.net). The GATK base quality score recalibrator was applied to correct sequencing artifacts. Genotyping was performed with GATK GenotypeGVCFs in the interval intersecting all the capture kits ±50 bp. Sample genotypes with a coverage <8×, a genotype quality (GQ)<20, or a ratio of reads for the less covered allele (reference or variant allele) over the total number of reads covering the position (minor read ratio, MRR)<20% were filtered out. We filtered out variant sites (i) with a call rate <50% in gnomAD genomes and exomes, (ii) a non-PASS filter in the gnomAD database, (iii) falling in low-complexity or decoy regions, (iv) that were multi-allelic with more than four alleles, (v) with more than 20% missing genotypes in our cohort, and (vi) spanning more than 20 nucleotides. Variant effects were predicted with the Ensembl Variant Effect Predictor (VEP) (67) and the Ensembl GRCh37.75 reference database, retaining the most deleterious annotation obtained from Ensembl canonical transcripts overlapping with RefSeq transcripts.

Statistical Analysis

We performed an enrichment analysis focusing on X chromosome genes on our cohort of 1,005 male patients with life-threatening COVID-19 pneumonia without known inborn errors of TLR3- and IRF7-dependent type I IFN immunity (8 and without neutralizing auto-Abs against type I IFNs (9), and 326 male individuals with asymptomatic or paucisymptomatic infection (Table 12). We considered variants that were predicted to be loss-of-function or missense, with a MAF below 0.0001 (gnomAD v2.1.1). We compared the proportion of patients and controls carrying at least one nonsynonymous variant by logistic regression and likelihood ratio tests. We accounted for the ethnic heterogeneity of the cohorts by including the first five principal components of the principal component analysis (PCA) in the logistic regression model. Analyses were also adjusted for age. We checked that our adjusted burden test was well-calibrated by also performing an analysis of enrichment in rare (MAF <0.0001) synonymous variants. PCA was performed with Plink v1.9 software on whole-exome and whole-genome sequencing data, with the 1000 Genomes (1 kG) Project phase 3 public database as a reference, using 18,917 exonic variants with a minor allele frequency >0.01 and a call rate >0.99.

Cell Culture

EBV-transformed B-lymphocyte (B-EBV) cell lines derived from the patients were grown in complete RPMI 1640 (Life Technologies) supplemented with 10% heat-inactivated fetal bovine serum (FBS). HEK293T cells, derived from the human embryonic kidney 293 cell line, which expresses a mutant version of the SV40 large T antigen, were grown in complete DMEM (Life Technologies) supplemented with 10% FBS. Cells were incubated at 37° C. in the presence of 5% CO₂.

Expression Vectors and Transfection Experiments

All the TLR7 variants in our analysis were generated by site-directed mutagenesis (Table 18). The WT or variant alleles were re-introduced into a Myc-DDK-pCMV6 vector (Origene). HEK293T cells, which have no endogenous TLR7 or TLR8 expression, were transfected with the Myc-DDK-pCMV6 vector, empty or containing the WT or a variant allele, in the presence of X-tremeGENE™ 9 DNA Transfection Reagent (Sigma-Aldrich), according to the manufacturer's instructions.

TABLE 18
TLR7 Variant Primer Sequences for the Site-Directed Mutagenesis.

| | Sequence 5′ to 3′ | |
|---|---|---|
| Variant | Forward primer | Reverse primer |
| D243E | TCCAAGAAGAGGATTTTAATAACCTCAACCAATTAC (SEQ ID | GTATTAAAATCCTCTTCTTGGATTTTTGCAATC (SEQ ID NO: 8) |
| | NO: 7) | |
| D244Y | CCAAGAAGATTATTTTAATAACCTCAACC (SEQ ID NO: 9) | GTTATTAAAATAATCTTCTTGGATTTTTGCAATC (SEQ ID NO: 10) |
| E617A | GGACCATGGCGAGTGAGTCTCTTAGAAC (SEQ ID NO: 11) | AGACTCACTCGCCATGGTCCTGCTGGTGG (SEQ ID NO: 12) |
| E654K | TAAAATTAGAGAAATTAGACATCTCTAAAAATTCCCTAAG | GTCTAATTTCTCTAATTTTAGCAGATTCTTGAATAATTG (SEQ ID |
| | (SEQ ID NO: 13) | NO: 14) |
| F310L | CCCCAAGATGGCTTAAGAACATCAACAAACTCCAG(SEQ ID | GATGTTCTTAAGCCATCTTGGGGGCACATGCTG (SEQ ID NO: 16) |
| | NO: 15) | |
| H630Y | CAGAGGAAATTACTTAGATGTTTTATGGAGAGAAG(SEQ ID | ACATCTAAGTAATTTCCTCTGAATTCCAGAGTTC (SEQ ID NO: 18) |
| | NO: 17) | |
| H781L | GCTTTTGCTTCATAATCGGTTTCTG (SEQ ID NO: 19) | GATTATGAAGCAAAAGCAACATCTTC (SEQ ID NO: 20) |
| I505T | AAATAGTACATTTTTTGTCAAGTCCTCTGATTTTCAG (SEQ ID | CAAAAAATGTACTATTTTTACTTAGATCCAAGGTCTG (SEQ ID |
| | NO: 21) | NO: 22) |
| I657T | GAGGAATTAGACACCTCTAAAAATTCCCTAAGTTTCTTGC (SEQ | GAATTTTTAGAGGTGTCTAATTCCTCTAATTTTAGCAGATTC (SEQ ID |
| | ID NO: 23) | NO: 24) |
| K684* | CTCTTTGGCCTAAAATGGGCTCAAATCTTTCAG(SEQ ID | GAGCCCATTTTAGGCCAAAGAGAGATTCTTTAGATTTGG (SEQ ID |
| | NO: 25) | NO: 26) |
| L134P | CCCTTTACCCGGATGGAAACCAGCTACTAG (SEQ ID NO: 27) | GGTTTCCATCCGGGTAAAGGGATTTTAAATAAG (SEQ ID NO: 28) |
| L227fs* | GCCATCTACTTAACAGAACTATATCTCTACAAC (SEQ ID | GATATAGTTCTGTTAAGTAGATGGCAAAACAGTAGG (SEQ ID NO: 30) |
| | NO: 29) | |
| L988S | GATTATCTCGATATTTCTTGAGAAG (SEQ ID NO: 31) | GAAATATCGAGATAATCACATCAAC (SEQ ID NO: 32) |
| N275T | CCGTGTAAAACTAATTCTCCCCTACAGATCC (SEQ ID | GGAGAATTAGTTTTACACGGCGCACAAGGAAATGG (SEQ ID NO: 34) |
| | NO: 33) | |
| N158Tfs*11 | CTCAGCCTTGAGGCAACAACATCTTTTCCATCAG (SEQ ID | AGATGTTGTTGCCTCAAGGCTGAGAAGCTG (SEQ ID NO: 36) |
| | NO: 35) | |
| P715S | GACCACTGTCTCTGAGAGATTATCCAACTGTTC (SEQ ID | GATAATCTCTCAGAGACAGTGGTCAGTTGGTTGTG (SEQ ID NO: 38) |
| | NO: 37) | |
| A819T | GCCAGGAACACACAAGGGCCAAAGTG (SEQ ID NO: 39) | CTTGTGTGTTCCTGGCCC GGCCCCACACAAG (SEQ ID NO: 40) |
| Q710Rfs* | CAGCCACAACCGACCACTGTCCCTGAGAGATTATC (SEQ ID | GACAGTGGTCGGTTGTGGCTGAGGTCCAAAG (SEQ ID NO: 42) |
| | NO: 41) | |
| V795F | CTGTGTGGTTTTTCTGGTGGGTTAACCATACG (SEQ ID | ACCCACCAGAAAAACCACACAGCATCACAGGTG (SEQ ID NO: 44) |
| | NO: 43) | |
| S301P | CAGTAACCCTCTTCAGCATGTGCCCCCAAG (SEQ ID NO: 45) | GCTGAAGAGGGTTACTGTGTAGACGTAAAACTTTTAATTC (SEQ ID |
| | | NO: 46) |
| A288V | ATGCTTTTGATGTGCTGACAGAATTAAAAGTTTTACG (SEQ ID | TTCTGTCAGCACATCAAAAGCATTTACAGGGATC (SEQ ID NO: 48) |
| | NO: 47) | |
| V219I | TGTCACAGCCATCCCTACTGTTTTGCCATCTAC (SEQ ID | CAGTAGGGATGGCTGTGACATTGTTATCTTTCAG (SEQ ID NO: 50) |
| | NO: 49) | |
| A448V | CTCAAATGTCAGAACTTCTGTAGAAAG (SEQ ID NO: 51) | AAGTTCTGACATTTGAGCAGAAGCCAAC (SEQ ID NO: 52) |
| R920K | AGACCCAAAAGAGAAACATTTTAATTTATGTCTCG (SEQ ID | TTCTCTTTTGGGTCTTCCAGTTTGGCCAC (SEQ ID NO: 54) |
| | NO: 53) | |
| A1032T | GTCTAAAGAACACCCTGGCCACAGACAATCATGTGG (SEQ ID | CTGTGGCCAGGGTGTTCTTTAGACACTGCCAGAAG (SEQ ID NO: 56) |
| | NO: 55) | |
| Q11L | GAAGAGACTAATTCTTATCCTTTTTAACATAATCC (SEQ ID | TAAGAATTAGTCTCTTCAGTGTCCACATTGG (SEQ ID NO: 58) |
| | NO: 57) | |
| V219I | TGTCACAGCCATCCCTACTGTTTTGCCATCTAC (SEQ ID | CAGTAGGGATGGCTGTGACATTGTTATCTTTCAG (SEQ ID NO: 60) |
| | NO: 59) | |
| A448V | CTCAAATGTCAGAACTTCTGTAGAAAG(SEQ ID NO: 61) | AAGTTCTGACATTTGAGCAGAAGCCAAC (SEQ ID NO: 62) |
| V222D | CCCTACTGATTTGCCATCTACTTTAACAGAACTATA (SEQ ID | AGATGGCAAATCAGTAGGGACGGCTGTGACATTG (SEQ ID NO: 64) |
| | NO: 63) | |
| A1032T | GTCTAAAGAACACCCTGGCCACAGACAATCATGTGG (SEQ ID | TGGCCAGGGTGTTCTTTAGACACTGCCAGAAG (SEQ ID NO: 66) |
| | NO: 65) | |
| R920K | AGACCCAAAAGAGAAACATTTTAATTTATGTCTCG (SEQ ID | TTCTCTTTTGGGTCTTCCAGTTTGGCCAC (SEQ ID NO: 68) |
| | NO: 67) | |
| V92I | GACCATCTGATAGAGATCGATTTCAGATGCAACTG (SEQ ID | GATCTCTATCAGATGGTCCAGTCTGTGAAAGG(SEQ ID NO: 70) |
| | NO: 69) | |
| I759V | AATAAAGTCCAGATGATCCAAAAGACCAGCTTCCC (SEQ ID | TCTGGACTTTATTTGAGCTGAGATCCAGATATCG (SEQ ID NO: 72) |
| | NO: 71) | |
| M236L | CTCTACAACAACTTGATTGCAAAAATCCAAGAAG (SEQ ID | GCAATCAAGTTGTTGTAGAGATATAGTTCTG (SEQ ID NO: 74) |
| | NO: 73) | |
| N350S | CTGTCTTTCAGTTTTGAACTTCAGGTCTATCGTGC (SEQ ID | GACCTGAAGTTCAAAACTGAAAGACAGATCCAATTG (SEQ ID NO: 76) |
| | NO: 75 ) | |
| K293E | CTGACAGAATTAGAAGTTTTACGTCTACACAGTAACTC (SEQ | GTAGACGTAAAACTTCTAATTCTGTCAGCGCATC (SEQ ID NO: 78) |
| | ID NO: 77) | |
| T127I | TAGTGGACTCATTTATTTAAAATCCCTTTACCTGGATG (SEQ | TAAATAAATGAGTCCACTAAAGCTTCTGGGTTTAATC (SEQ ID |
| | ID NO: 79) | NO: 80) |
| I142M | CTAGAGATGCCGCAGGGCCTC(SEQ ID NO: 81) | CTGCGGCATCTCTAGTAGCTGGTTTCCATC (SEQ ID NO: 82) |
| L146F | GCAGGGCTTCCCGCCTAGCTTAC (SEQ ID NO: 83) | GCGGGAAGCCCTGCGGTATCTCTA (SEQ ID NO: 84) |
| A83V | CTCCCCAGTGTCCTTTCACAGACTGGACCATC (SEQ ID | AAGGACACTGGGGAGATGTCTGGTATGTGG (SEQ ID NO: 86) |
| | NO: 85) | |
| L988S | GATTATCTCGATATTTCTTGAGAAG (SEQ ID NO: 87) | GAAATATCGAGATAATCACATCAAC |
| | | (SEQ ID NO: 88) |
| V222L | GCCGTCCCTACTCTTTTGCCATCTACTTTAACAG (SEQ ID | GATGGCAAAAGAGTAGGGACGGCTGTG (SEQ ID NO: 90) |
| | NO: 89) | |
| H86L | CGTCCTTTCTCAGACTGGACCATCTGGTAG (SEQ ID NO: 91) | CAGTCTGAGAAAGGACGCTGGGGAG (SEQ ID NO: 92) |
| V283A | CAGATCCCTGCAAATGCTTTTGATGCGCTG (SEQ ID NO: 93) | AGCATTTGCAGGGATCTGTAGGGGAG (SEQ ID NO: 94) |
| R883H | GTATCAGCATCTAATATCACCAGACTGTTGC (SEQ ID | CTGGTGATATTAGATGCTGATACCCCTTTATCTTG (SEQ ID NO: 96) |
| | NO: 95) | |
| H55Q | CACAGACAAGCAATTGACAGAAATTCCTGGAG (SEQ ID | CTGTCAATTGCTTGTCTGTGCAGTCCAC (SEQ ID NO: 98) |
| | NO: 97) | |
| E617A | GGACCATGGCGAGTGAGTCTCTTAGAAC (SEQ ID NO: 99) | AGACTCACTCGCCATGGTCCTGCTGGTGG (SEQ ID NO: 100) |
| Q117K | CAAGAGGCTGAAGATTAAACCCAGAAGCTTTAG (SEQ ID | GTTTAATCTTCAGCCTCTTGATGCACATGTTG (SEQ ID NO: 102) |
| | NO: 101) | |
| T858K | GTGATGATGAAAGCAAGTCACCTCTATTTCTG (SEQ ID | GACTTGCTTTCATCATCACCATGAGAAAGAG (SEQ ID NO: 104) |
| | NO: 103) | |
| N720S | TTATCCAGCTGTTCCAGAAGCCTCAAGAATCTG (SEQ ID | GAACAGCTGGATAATCTCTCAGGGACAGTGGTC (SEQ ID NO: 106) |
| | NO: 105) | |
| S582L | GCCATTATTTTCAATTAGAAGGAATTACTCATATGC (SEQ ID | GAGTAATTCCTTCTAATTGAAAATAATGGCTATTACTGC (SEQ ID |
| | NO: 107) | NO: 108) |
| A482T | CAAAGAGACTTCTTTCATGTCTGTTAATGAAAGCTG (SEQ ID | CATGAAAGAAGTCTCTTTGTTTTTGAATCTGC (SEQ ID NO: 110) |
| | NO: 109) | |
| D438V | CTTCAGGAGTTTCAAGTGAAGTTGGCTTCTGC (SEQ ID | GCCAACTTCACTTGAAACTCCTGAAGGTGATATTTTATTC (SEQ ID |
| | NO: 111) | NO: 112) |
| S663R | CTAAAAATTCCCTAAGGTTCTTGCCTTCTGGAG(SEQ ID | CAGAAGGCAAGAACCTTAGGGAATTTTTAGAGATGTC (SEQ ID |
| | NO: 113) | NO: 114) |
| F388L | GAGTTGAAAAGCCTTAACCTCTCGCCATTAC (SEQ ID | CGAGAGGTTAAGGCTTTTCAACTCTTTAAAGAC (SEQ ID NO: 116) |
| | NO: 115) | |
| T221del | GTCACAGCCGTCCCTGTTTTGCCATCTAC (SEQ ID NO: 117) | GATGGCAAAACAGGGACGGCTGTGACATTGTTATC (SEQ ID NO: 118) |
| L557F | CGGCTTGATTTATTCCATTCAACAGCATTTG (SEQ ID | GTTGAATGGAATAAATCAAGCCGGTTGTTGGAG (SEQ ID NO: 120) |
| | NO: 119) | |
| I113F | CAACATGTGCTTCAAGAGGCTGCAG(SEQ ID NO: 121) | GCCTCTTGAAGCACATGTTGTTTTTTGAC (SEQ ID NO: 122) |
| D332G | GAAATTGGGGGTGCTAAATTTCTGCATTTTCTC (SEQ ID | ATTTAGCACCCCCAATTTCTTTGGCCAAGAAG (SEQ ID NO: 124) |
| | NO: 123) | |
| K993T | CTTGAGACGCCCTTTCAGAAGTCCAAG (SEQ ID NO: 125) | GACTTCTGAAAGGGCGTCTCAAGAAATATCAAG (SEQ ID NO: 126) |
| L1001I | CAAGTTCATCCAGCTCCGGAAAAGGC (SEQ ID NO: 127) | CGGAGCTGGATGAACTTGGACTTCTGAAAG (SEQ ID NO: 128) |
| R357C | CAGGTCTATTGTGCATCTATGAATCTATCACAAG (SEQ ID | GATTCATAGATGCACAATAGACCTGAAGTTCAAAATTG (SEQ ID |
| | NO: 129) | NO: 130) |
| I174M | CTGGCCAACATGGAAATACTCTACCTGGGC (SEQ ID | GTAGAGTATTTCCATGTTGGCCAGTTCTGTTAGATTC (SEQ ID |
| | NO: 131) | NO: 132) |
| E929Q | TATGTCTCCAGGAAAGGGACTGGTTACCAG (SEQ ID | CAGTCCCTTTCCTGGAGACATAAATTAAAATGTTTCTC (SEQ ID |
| | NO: 133) | NO: 134) |
| R750Q | GCCTTCCAGTTGCAATATCTGGATCTCAGCTC (SEQ ID | GATCCAGATATTGCAACTGGAAGGCATCTTG (SEQ ID NO: 136) |
| | NO: 135) | |
| R357H | CAGGTCTATCATGCATCTATGAATCTATCACAAGC (SEQ ID | CATAGATGCATGATAGACCTGAAGTTCAAAATTG(SEQ ID NO: 138) |
| | NO: 137) | |
| L909S | GAGTGGGTTTCGGCTGAGCTGGTGGC (SEQ ID NO: 139) | CTCAGCCGAAACCCACTCGGTCACAG (SEQ ID NO: 140) |
| A327D | CTTCTTGGACAAAGAAATTGGGGATGC (SEQ ID NO: 141) | CAATTTCTTTGTCCAAGAAGTTTTGGGACAGATC (SEQ ID NO: 142) |
| K334E | GATGCTGAATTTCTGCATTTTCTCCCCAGC (SEQ ID | GCAGAAATTCAGCATCCCCAATTTCTTTGGC (SEQ ID NO: 144) |
| | NO: 143) | |
| P4L | TGGTGTTTCTAATGTGGACACTGAAGAGAC (SEQ ID | GTCCACATTAGAAACACCATGAATTCCCGGC (SEQ ID NO: 146) |
| | NO: 145) | |
| T1048M | CAAGGAAATGGTCTAGCCGCGGTCATAG (SEQ ID NO: 147) | CGGCTAGACCATTTCCTTGAACACCTGAC (SEQ ID NO: 148) |
| Y468H | CATTATTTCAGACATGATAAGTATGCAAGGAGTTGCAG (SEQ | GCATACTTATCATGTCTGAAATAATGTAATTGTTCCAGGAC (SEQ ID |
| | ID NO: 149) | NO: 150) |
| R422S | CAATTTAAAAGCCTGAAAGTCATAGATCTTTCAGTGA (SEQ ID | GACTTTCAGGCTTTTAAATTGTTTAAACATGCTG(SEQ ID NO: 152) |
| | NO: 151) | |
| V430L | GTCATAGATCTTTCACTGAATAAAATATCACCTTCAGGAG (SEQ | GTGATATTTTATTCAGTGAAAGATCTATGACTTTCAGTC (SEQ ID |
| | ID NO: 153) | NO: 154) |
| H1038Y | CACAGACAATTATGTGGCCTATAGTCAGGTG (SEQ ID | CCACATAATTGTCTGTGGCCAGGGCG (SEQ ID NO: 156) |
| | NO: 155) | |
| P457S | GTTATGAATCCCAGGTCCTGGAACAATTAC (SEQ ID | CAGGACCTGGGATTCATAACTTTCTACAGAAGTTC (SEQ ID NO: 158) |
| | NO: 157) | |
| R473K | GATAAGTATGCAAAGAGTTGCAGATTCAAAAACAAAGAGG (SEQ | GAATCTGCAACTCTTTGCATACTTATCATATCTG (SEQ ID NO: 160) |
| | ID NO: 159) | |
| T1035I | CTGGCCATAGACAATCATGTGGCCTATAG (SEQ ID NO: 161) | GATTGTCTATGGCCAGGGCGTTCTTTAG (SEQ ID NO: 162) |
| A448T | CTGCTCAAATACCAGAACTTCTGTAGAAAG (SEQ ID | GAAGTTCTGGTATTTGAGCAGAAGCCAACTTC (SEQ ID NO: 164) |
| | NO: 163) | |
| R449K | GCTCAAATGCCAAAACTTCTGTAGAAAGTTATGAAC (SEQ ID | CAGAAGTTTTGGCATTTGAGCAGAAGCCAAC (SEQ ID NO: 166) |
| | NO: 165 | |
| V442D | CAAGTGAAGATGGCTTCTGCTCAAATGC (SEQ ID NO: 167) | GCAGAAGCCATCTTCACTTGAATCTCCTGAAG(SEQ ID NO: 168) |
| G638S | ATGGAGAGAAAGTGATAACAGATACTTACAATTATTCAAG (SEQ | CTGTTATCACTTTCTCTCCATAAAACATCTAAGTG (SEQ ID NO: 170) |
| | ID NO: 169) | |
| T712N | CAACCAACTGAACACTGTCCCTGAGAGATTATC (SEQ ID | GACAGTGTTCAGTTGGTTGTGGCTGAGGTC (SEQ ID NO: 172) |
| | NO: 171) | |
| T712S | CAACCAACTGAGCACTGTCCCTGAGAGATTATC (SEQ ID | GACAGTGCTCAGTTGGTTGTGGCTGAGGTC (SEQ ID NO: 174) |
| | NO: 173) | |
| S724R | CTGTTCCAGACGCCTCAAGAATCTGATTC (SEQ ID NO: 175) | CTTGAGGCGTCTGGAACAGTTGGATAATCTC (SEQ ID NO: 176) |
| G145D | GCAGGACCTCCCGCCTAGCTTAC (SEQ ID NO: 177) | CGGGAGGTCCTGCGGTATCTCTAGTAG (SEQ ID NO: 178) |
| D829N | GATCTCCCTGAATCTGTACACCTGTGAGTTAGATC (SEQ ID | GTACAGATTCAGGGAGATCACACTTTGGC (SEQ ID NO: 180) |
| | NO: 179) | |
| A819T | GCCAGGAACACACAAGGGCCAAAGTG (SEQ ID NO: 181) | CTTGTGTGTTCCTGGCCCCACACAAG(SEQ ID NO: 182) |
| D632E | CACTTAGAAGTTTTATGGAGAGAAGGTGATAACAG (SEQ ID | CTCCATAAAACTTCTAAGTGATTTCCTCTGAATTCCAG (SEQ ID |
| | NO: 183) | NO: 184) |
| L153I | CTTACAGCTTATCAGCCTTGAGGCCAAC (SEQ ID NO: 185) | CTCAAGGCTGATAAGCTGTAAGCTAGGCG (SEQ ID NO: 186) |
| L140I | CAGCTAATAGAGATACCGCAGGGCCTC(SEQ ID NO: 187) | GCGGTATCTCTATTAGCTGGTTTCCATCCAG (SEQ ID NO: 188) |
| T65M | GTATTCCCATGAACACCACGAACCTCAC (SEQ ID NO: 189) | GTGGTGTTCATGGGAATACCTCCAGGAATTTC (SEQ ID NO: 190) |
| L177F | CAACATAGAAATATTCTACCTGGGCCAAAACTG (SEQ ID | CAGGTAGAATATTTCTATGTTGGCCAGTTCTG (SEQ ID NO: 192) |
| | NO: 191) | |
| Q138R | GATGGAAACCGGCTACTAGAGATACCGCAG (SEQ ID | GTATCTCTAGTAGCCGGTTTCCATCCAGGTAAAG (SEQ ID NO: 194) |
| | NO: 193 | |
| E175Q | GCCAACATACAAATACTCTACCTGGGCCA GCCAAAACTG (SEQ ID | GTAGAGTATTTGTATGTTGGCCAGTTCTGTTAGATTC (SEQ ID |
| | NO: 195) | NO: 196) |
| I195T | CATATTCAACAGAGAAAGATGCCTTCCTAAACTTG (SEQ ID | GAAGGCATCTTTCTCTGTTGAATATGAAACATAACAAG (SEQ ID |
| | NO: 197) | NO: 198) |
| E170Q | GAGAATCTAACACAACTGGCCAACATAGAAATACTCTAC (SEQ | GTTGGCCAGTTGTGTTAGATTCTCTTTTCTG (SEQ ID NO: 200) |
| | ID NO: 199) | |
| L25P | CAAACTCCCTGGGGCTAGATGGTTTCCTA (SEQ ID NO: 201) | CTAGCCCCAGGGAGTTTGGAAATTAGGATTATG (SEQ ID NO: 202) |
| A809V | CTTACCTGGTCACAGATGTGACTTGTGTG (SEQ ID NO: 203) | CACATCTGTGACCAGGTAAGGAATAGTCACCTC (SEQ ID NO: 204) |
| E543Q | CAACCTTTAGCACAGCTGAGATATTTGGACTTCTC (SEQ ID | CTCAGCTGTGCTAAAGGTTGGAATTCAC(SEQ ID NO: 206) |
| | NO: 205) | |
| E242del | GATTGCAAAAATCCAAGATGATTTTAATAACCTCAACC (SEQ | GAGGTTATTAAAATCATCTTGGATTTTTGCAATCATGTTGTTG (SEQ ID |
| | ID NO: 207) | NO: 208) |
| E242D | ATCATCATCTTGGATTTTTGCAATCATGTTGTTG (SEQ ID | ATCATCATCTTGGATTTTTGCAATCATGTTGTTG (SEQ ID NO: 210) |
| | NO: 209) | |
| I847T | CACTTTCCACATCTGTATCTCTCTTTCTCATGGTG (SEQ ID | GAGATACAGATGTGGAAAGTGAGAACAGAATCAGGTTAG (SEQ ID |
| | NO: 213) | NO: 212) |
| Y546del | CAGAGCTGAGATTGGACTTCTCCAACAACCG (SEQ ID | GTTGGAGAAGTCCAATCTCAGCTCTGCTAAAGGTTG(SEQ ID |
| | NO: 213) | NO: 214) |
| L1029P | GCAGTGTCCAAAGAACGCCCTGGC (SEQ ID NO: 215) | GCGTTCTTTGGACACTGCCAGAAGTATG (SEQ ID NO: 216) |
| S618N | GACCATGGAGAATGAGTCTCTTAGAACTCTGGA (SEQ ID | AGAGACTCATTCTCCATGGTCCTGCTG (SEQ ID NO: 218) |
| | NO: 217) | |
| P31S | CTAGATGGTTTTCTAAAA | CAGAGTTTTAGAAAACCATCTAGCCCCAAGGAG (SEQ ID NO: 220) |
| | TAAAACTCTGCCCTGTGATG (SEQ ID | |
| | NO: 219) | |
| P307Q | CATGTGCCCCAAAGATGGTTTAAGAACATCAAC (SEQ ID | CCATCTTTGGGGCACATGCTGAAGAGAG (SEQ ID NO: 222) |
| | NO: 221) | |
| H46R | CAAAGAACCGTGTGATCGTGGACTGCAC (SEQ ID NO: 223) | CGATCACACGGTTCTTTGGAACATCCAGAG (SEQ ID NO: 224) |
| D41H | GTCACTCTGCATGTTCCAAAGAACCATGTGATC (SEQ ID | GAACATGCAGAGTGACATCACAGGGCAG (SEQ ID NO: 226) |
| | NO: 225) | |
| K44R | GATGTTCCAAGGAACCATGTGATCGTGGAC (SEQ ID | CGATCACATGGTTCCTTGGAACATCCAGAGTGAC (SEQ ID NO: 228) |
| | NO: 227) | |
| M854I | CTTTCTCATAGTGATGATGACAGCAAGTC (SEQ ID NO: 229) | CATCATCACTATGAGAAAGAGAGATACAG (SEQ ID NO: 230) |
| V38I | GCCCTGTGATATCACTCTGGATGTTCCAAAG (SEQ ID | CATCCAGAGTGATATCACAGGGCAGAGTTTTAG (SEQ ID NO: 232) |
| | NO: 231) | |
| H298R | CGTCTACGCAGTAACTCTCTTCAGCATGTG (SEQ ID | GAAGAGAGTTACTGCGTAGACGTAAAACTTTTAATTCTGTC (SEQ ID |
| | NO: 233) | NO: 234) |
| N109Tfs*21 | CTGGGGTCAAAAACAACATGTGCATCAAGAGGCTG (SEQ ID | GCACATGTTGTTTTTGACCCCAGTGGAATAGGTAC (SEQ ID NO: 236) |
| | NO: 235) | |
| G331V | GCCAAAGAAATTGTGGATGCTAAATTTCTGCA (SEQ ID | GCATCCACAATTTCTTTGGCCAAGAAG (SEQ ID NO: 238) |
| | NO: 237) | |
| R296C | GTTTTATGTCTACACAGTAACTCTCTTC (SEQ ID NO: 239) | TGTGTAGACATAAAACTTTTAATTCTGTC (SEQ ID NO: 240) |
| G331W | GCCAAAGAAATTTGGGATGCTAAATTTCTGCA (SEQ ID | GCATCCCAAATTTCTTTGGCCAAGAAG (SEQ ID NO: 242) |
| | NO: 241) | |
| A288V | TTCTGTCAGCACATCAAAAGCATTTACAGGGATC (SEQ ID | ATGCTTTTGATGTGCTGACAGAATTAAAAGTTTTACG (SEQ ID |
| | NO: 243) | NO: 244) |
| D866N | CTATTTCTGGAATGTGTGGTATATTTACCATTTCTG (SEQ ID | CACACATTCCAGAAATAGAGGTGACTTGC (SEQ ID NO: 246) |
| | NO: 245) | |
| L916M | GCCAAAATGGAAGACCCAAGAGAG (SEQ ID NO: 247) | GTCTTCCATTTTGGCCACCAGCTCAGC (SEQ ID NO: 248) |
| V913M | CTGAGCTGATGGCCAAACTGGAAGAC (SEQ ID NO: 249) | GTTTGGCCATCAGCTCAGCCAAAAC (SEQ ID NO: 250) |
| A962T | GACAGACAAGTATACAAAGACTGAAAATTTTAAGATAGCA (SEQ | TCAGTCTTTGTATACTTGTCTGTCATCACAAAC (SEQ ID NO: 252) |
| | ID NO: 251) | |
| D959N | GTGATGACAAACAAGTATGCAAAGACTGA (SEQ ID NO: 253) | GCATACTTGTTTGTCATCACAAACACTGTC (SEQ ID NO: 254) |
| M957V | CAGTGTTTGTGGTGACAGACAAGTATGCAAAG (SEQ ID | CTTGTCTGTCACCACAAACACTGTCTTTTTGC (SEQ ID NO: 256) |
| | NO: 255) | |
| P272S | CTTGTGCGTCGTGTAAAAATAATTCTCCCCTAC (SEQ ID | TACACGACGCACAAGGAAATGGGGC (SEQ ID NO: 258) |
| | NO: 257) | |
| A1021V | CCGCAAGTTCACCCATACTTCTGGCA (SEQ ID NO: 259) | ATGGGTGAACTTGCGGGTTTGTTGGC (SEQ ID NO: 260) |
| F894C | TGCTTGTATTGTGTATGACACTAAAGACCCAGC (SEQ ID | CATACACAATACAAGCATCATAGCAACAGTCTGGTG (SEQ ID |
| | NO: 261) | NO: 262) |
| L1001H | CAAGTTCCACCAGCTCCGGAAAAGGC (SEQ ID NO: 263) | GAGCTGGTGGAACTTGGACTTCTGAAAG (SEQ ID NO: 264) |
| K999R | GAAGTCCAGGTTCCTCCAGCTCCGGAAAAG (SEQ ID | GGAGGAACCTGGACTTCTGAAAGGGCTTC (SEQ ID NO: 266) |
| | NO: 265) | |
| L680I | CTAAAGAATCTATCTTTGGCCAAAAATGGGCTC (SEQ ID | GCCAAAGATAGATTCTTTAGATTTGGAGGCATACCATC (SEQ ID |
| | NO: 267) | NO: 268) |
| F200L | GAGAAAGATGCCCTCCTAAACTTGACAAAGTTAAAAGTG (SEQ | GTCAAGTTTAGGAGGGCATCTTTCTCTATTGAATATG (SEQ ID |
| | ID NO: 269) | NO: 270) |
| K764M | GATGATCCAAATGACCAGCTTCCCAGAAAATG | GAAGCTGGTCATTTGGATCATCTGGATTTTA |
| | GTCAAGTTTAGGAGGGCATCTTTCTCTATTGAATATG (SEQ ID | GTCAAGTTTAGGAGGGCATCTTTCTCTATTGAATATG (SEQ ID |
| | NO: 271) | NO: 272) |
| E2910 | GATGCGCTGACACAATTAAAAGTTTTACGTCTACACAG | CTTTTAATTGTGTCAGCGCATCAAAAGCATTTAC |
| | GTCAAGTTTAGGAGGGCATCTTTCTCTATTGAATATG (SEQ ID | GTCAAGTTTAGGAGGGCATCTTTCTCTATTGAATATG (SEQ ID |
| | NO: 273) | NO: 274) |
| InFus.pCMV6- | GGGCGGCCGGGAATTCATGGTGTTTCCAATGTGGACACT | |
| ECORI-F | GTCAAGTTTAGGAGGGCATCTTTCTCTATTGAATATG (SEQ ID | |
| | NO: 275) | |
| InFus.pCMV6- | | GAAACAGCTATGACCGCGGCTAGACCGTTTCCTTGAACACCT |
| SACTT-R | | GTCAAGTTTAGGAGGGCATCTTTCTCTATTGAATATG (SEQ ID |
| | | NO: 276) |

Western Blotting

For whole-cell extracts, the cells were lysed by incubation in the following buffer (50 mM Tris-HCl, pH 8.0, 150 mM NaCl, 1% NP40), supplemented with a mixture of protease inhibitors (Sigma-Aldrich), for 30 minutes at 4° C. The lysates were then centrifuged at 21,000×g for 20 minutes at 4° C. The supernatants were processed directly for western blotting. Western blotting was performed on 10 pg of total extract from transfected HEK293T cells, with monoclonal antibodies specific for the leucine-rich repeats within the human TLR7 protein (Cell Signaling Technology).

RNA Extraction and Reverse Transcription-Quantitative PCR (RT-qPCR)

Total RNA was extracted with the RNeasy Mini Kit (Qiagen), according to the manufacturer's instructions. Reverse transcription was performed on 1 μg of RNA with random primers and the SuperScript® III reverse transcriptase (Invitrogen), according to the manufacturer's protocol. Quantitative PCR was then performed with the TaqMan™ Fast Universal PCR Master Mix (2×) and the FAM-MGB TaqMan TNF exons 1-2 (Hs99999-43_ml) probes. The VIC-TAMRA probe for GUSB (Applied Biosystems, Cat: 4310888E) was used as an endogenous control. Real-time PCR amplification was monitored with the 7500 Fast Real-Time PCR System (Applied Biosystems). Relative expression levels were determined according to the ΔCt method.

Luciferase Reporter Assay

HEK293T cells, which have no endogenous TLR7 expression, were transfected with the pCMV6 vector bearing wild-type or variant TLR7 (50 ng), the reporter construct pGL4.32 (100 ng), and an expression vector for Renilla luciferase (10 ng), with the X-tremeGENE™ 9 DNA Transfection Reagent kit (Sigma-Aldrich). The pGL4.32 [luc2P/NF-κB-RE/Hygo] (Promega) reporter vector contains five copies of the NF-κB-responsive element (NF-κB-RE) linked to the luciferase reporter gene luc2P. After 24 hours, the transfected cells were left unstimulated or were stimulated with 1 μg/mL R848 (Resquimod), for activation via TLR7/8 (Invivogen), or 5 μg/mL R837 (Imiquimod) (Invivogen), or 5 μg/mL CL264 (Invivogen), human TLR7-specific agonists, for 24 hours. Relative luciferase activity was then determined by normalizing the values against the firefly:Renilla luciferase signal ratio.

ELISA Analysis of TNF Production in B-EBV Cells

ELISA was performed as previously described (68). We suspended 1×10⁶ EBV-B cells per well in RPMI 1640 supplemented with 10% FBS. The cells were activated by incubation with 1 μg/mL R848, and 5 μg/mL imiquimod for 24 hours. The supernatants were harvested after 24 hours of activation. ELISA determinations of TNF in cell culture supernatants were performed with a kit (Thermo Fisher Scientific), according to the manufacturer's instructions.

Stable Transduction

The WT coding sequence of TLR7 was inserted into pTRIP-CMV-puro-2A. For lentivirus production, HEK293T cells were transfected with 1.6 g pTRIP-CMV-puro-2A-TLR7-WT (or Mutant: K684*), 0.2 μg pCMV-VSV-G (Addgene), 0.2 μg pHXB2 (NIH-AIDS Reagent 22 Program) and 1 g psPAX2 (Addgene), with X-treme gene 9 (Roche), according to the manufacturer's instructions. Supernatants were harvested after 24 hours and 8 μg/mL protamine sulfate was added. The lentiviral suspension obtained was used to transduce 2×10⁵ EBV-B cells by spinoculation at 1,200×g for 2 hours. The transduced cells were selected by incubation on medium containing 1 μg/mL puromycin for two days. The cells were then selected by incubation for a further two days on medium containing 2 μg/mL puromycin. During viral transduction, the cells were cultured with 5 M IRAK4 inhibitor (PF06650833) (Bio-techne) to prevent cell death due to the overproduction of TLR7. Selected transduced cells were then stimulated with 1 μg/mL R848 or 5 μg/mL imiquimod for 24 hours without IRAK4 inhibitor. The supernatants were harvested after 24 hours of activation. ELISA determinations of TNF in cell culture supernatants were performed with a kit (Thermo Fisher Scientific), according to the manufacturer's instructions.

Deep Immunophenotyping by Mass Cytometry (CyTOF)

CyTOF was performed on whole blood with the Maxpar Direct Immune Profiling Assay (Fluidigm), according to the manufacturer's instructions. Cells were frozen at −80° C. after overnight staining to eliminate dead cells, and acquisition was performed on a Helios machine (Fluidigm). All the samples were processed within 24 hours of sampling. Data analysis was performed with OMIQ software.

PBMC enrichment using MACS system

Blood were collected from two healthy individuals and separated by the concentration gradient method with Ficoll® Paque Plus (Cytiva). After isolations of PBMCs, leucocyte subset (T cell, B cell, monocyte, pDC, and mDC) were purified by negative selection using MACS beads system (Milteni Biotec). Cells were plated into a U-bottomed 96-well plate at a density of 2×10⁴ cells/well for T cells, B cells, monocytes, pDCs, or mDCs in 200 μL/well RPMI-1640 with GlutaMAX supplemented with 10% FBS or 10×10⁴ cells/well for whole blood and PBMCs. Cells were left unstimulated or stimulated with 1 g/mL CL264, 100 ng/ml TL8-506 (Invivogen), 1 g/mL R848, 2 M CpG-c (Invivogen), or 12.5 ng/ml PMA and 0.125 M ionomycin for 24 hours. The supernatants were harvested after 24 hours of activation. Cytokines production were determined by ELISA (IFN-α—PBL Assay Science, IFN-β—PBL Assay Science, IFN-λ1 (IL-29)—Invivogen, IFN-ω—Invitrogen or IL-8—R&D SYSTEMS); according to the manufacturer's instructions.

Analysis for TLR7 and TLR8 Expression Pattern in PBMCs by Flow Cytometry

Freshly thawed PBMCs from healthy donors were dispensed into a V-bottomed 96-well plate at a density of 1×10⁶ cells/well, in 200 μL PBS/well. In brief, cells were stained by incubation with the LIVE/DEAD fixable blue dead-cell staining kit (Thermo Fisher Scientific, 1:800) and FcR blocking reagent (Miltenyi Biotec, 1:25) on ice for 15 min. For surface staining, cells were incubated with anti-γδTCR-BUV611 (BD Biosciences, 1:50), anti-CD183-BV750 (BD Biosciences, 1:20), and anti-CD194-BUV615 (BD Biosciences, 1:20) antibodies on ice for 30 min in 0.1% BSA and 0.01% sodium azide in PBS. They were then incubated with anti-CD141-BB515 (BD Biosciences, 1:40), anti-CD57-FITC (Biolegend, 1:83), anti-TCR Vδ2-PerCP (Biolegend, 1:166), anti-TCR Vα7.2-PerCP/Cyanine5.5 (Biolegend, 1:40), anti-TCR Vd1-PerCP-Vio 700 (Miltenyi Biotec, 1:100), anti-CD14-Spark Blue 550 (Biolegend, 1:40), anti-CD1c-Alexa Fluor 647 (Biolegend, 1:50), anti-CD38-APC/Fire 810 (Biolegend, 1:30), anti-CD27-APC-H7 (BD Biosciences, 1:50), anti-CD127-APC-R700 (BD Biosciences, 1:50), anti-CD19-Spark NIR 685 (Biolegend, 1:83), anti-CD45RA-BUV395 (BD Biosciences, 1:83), anti-CD16-BUV496 (BD Biosciences, 1:166), anti-CD11b-BUV563 (BD Biosciences, 1:100), anti-CD56-BUV737 (BD Biosciences, 1:83), anti-CD8-BUV805 (BD Biosciences, 1:83), anti-hMR1-BV421 (NIH tetramer facility, 1:100), anti-CD11c-BV480 (BD Biosciences, 1:40), anti-CD45-BV510 (Biolegend, 1:83), anti-CD33-BV570 (Biolegend, 1:83), anti-iNKT-BV605 (Biolegend, 1:25), anti-CD161-BV650 (BD Biosciences, 1:25), anti-CCR6-BV711 (Biolegend, 1:83), anti-CCR7—BV785 (Biolegend, 1:40), anti-CD3-Pacific Blue (Biolegend, 1:83), anti-CD20-Pacific Orange (Life Technologies, 1:50), anti-CD123-Super Bright 436 (Invitrogen, 1:40), anti-CD24-PE-Alexa Fluor 610 (Life Technologies, 1:25), anti-CD25-PE-Alexa Fluor 700 (Life Technologies, 1:25), anti-CD294-Biotin (Invitrogen, 1:50), anti-CD209-PE/Cyanine7 (Biolegend, 1:25), anti-CD117-PE/Dazzle 594 (Biolegend, 1:83), anti-HLA-DR-PE/Dazzle 810 (Biolegend, 1:50), and anti-CD4-cFluor™ YG584 (Cytek, 1:83) antibodies on ice for at least 30 min. The cells were then washed and stained by incubation with streptavidin-PE/Cy5 (Biolegend, 1:3000) on ice for 30 min. The cells were then fixed and permeabilized for intracellular staining with anti-hTLR7-PE (R&D Systems) and anti-TLR8-APC (Biolegend) antibodies, with the eBioscience Foxp3/Transcription Factor Staining Buffer Set (Invitrogen), according to the manufacturer's instructions. The cells were then washed and acquired with a five-laser Cytek Aurora (Cytek) flow cytometer.

pDC Activation

Freshly sorted pDCs were cultured in 96-well plates at a concentration of 5×10⁵ cells per mL in the presence of medium alone (RPMI 1640 Medium with GlutaMAX, 10% FBS, 1% MEM NEAA, 1% sodium pyruvate, and 1% penicillin/streptomycin), CL264 (Invivogen, 1 pg/mL), or the SARS-CoV-2 primary strain 22095 (44) at a multiplicity of infection (MOI) of 1. After 24 h of culture, the pDC supernatant was collected for cytokine quantification, and the PDCs were collected for diversification assessment by flow cytometry. In some experiments, RNA was purified from the pDCs with the RNeasyMicro kit (Qiagen) and IFN responses were analyzed by RNAseq (low-input SMARTer kit (Takara) and sequencing on an Illumina NextSeq 500).

Flow Cytometry Analysis for Human pDCs

For assessments of pDC diversification, cells were stained with Zombie Violet fixable viability dye (Biolegend), BV711 anti-CD123 (Biolegend, clone 6H6), PE anti-CD80 (BD, clone L307.4), and PerCP-efluor 710 anti-PD-L1 (eBioscience, clone MIH1) antibodies. Data were acquired with an LSR Fortessa (BD Biosciences) flow cytometer, and analyzed with FlowJo software (Tree Star). Flow cytometry analyses were performed at the flow cytometry core facility of IRSL (Paris, France).

Determination of Secreted Inflammatory Cytokines

We measured the production, by pDCs, of IFN-α2, IL-8, IL-6, and IP-10, by determining the levels of these cytokines in culture supernatants with the BD cytometric bead array (CBA), according to the manufacturer's protocol, with a limit of detection of 20 pg/mL. Acquisitions were performed on an LSR Fortessa (BD Biosciences) flow cytometer, and cytokine concentrations were determined with FCAP Array Software (BD Biosciences).

### REFERENCES

1. J. L. Casanova, H. C. Su, C. H. G. Effort, A Global Effort to Define the Human Genetics of Protective Immunity to SARS-CoV-2 Infection. Cell 181, 1194-1199 (2020).
2. Q. Zhang et al., Life-Threatening COVID-19: Defective Interferons Unleash Excessive Inflammation. Med (N Y) 1, 14-20 (2020).
3. M. O'Driscoll et al., Age-specific mortality and immunity patterns of SARS-CoV-2. Nature 590, 140-145 (2021).
4. E. P. Scully, J. Haverfield, R. L. Ursin, C. Tannenbaum, S. L. Klein, Considering how biological sex impacts immune responses and COVID-19 outcomes. Nature Reviews Immunology 20, 442-447 (2020).
5. M. J. Ciancanelli et al., Infectious disease. Life-threatening influenza and impaired interferon amplification in human IRF7 deficiency. Science (New York, N.Y.) 348, 448-453 (2015).
6. N. Hernandez et al., Life-threatening influenza pneumonitis in a child with inherited IRF9 deficiency. The Journal of Experimental Medicine 215, 2567-2585 (2018).
7. H. K. Lim et al., Severe influenza pneumonitis in children with inherited TLR3 deficiency. The Journal of Experimental Medicine 216, 2038-2056 (2019).
8. Q. Zhang et al., Inborn errors of type I IFN immunity in patients with life-threatening COVID-19. Science (New York, N.Y.) 370, (2020).
9. P. Bastard et al., Autoantibodies against type I IFNs in patients with life-threatening COVID-19. Science (New York, N.Y.) 370, (2020).
10. E. Y. Wang et al., Diverse Functional Autoantibodies in Patients with COVID-19. Nature, (2021).
11. R. Koning et al., Autoantibodies against type I interferons are associated with multi-organ failure in COVID-19 patients. Intensive care medicine, 1-3 (2021).
12. J. Troya et al., Neutralizing Autoantibodies to Type I IFNs in >10% of Patients with Severe COVID-19 Pneumonia Hospitalized in Madrid, Spain. Journal of Clinical Immunology, 1-9 (2021).
13. G. David et al., Antibodies against type-I Interferon: detection and association with severe clinical outcome in COVID-19 patients. medRxiv, 2021.2004.2002.21253262 (2021).
14. M. G. P. van der Wijst et al., Longitudinal single-cell epitope and RNA-sequencing reveals the immunological impact of type 1 interferon autoantibodies in critical COVID-19. bioRxiv (2021).
15. S. E. Vazquez et al., Neutralizing Autoantibodies to Type I Interferons in COVID-19 Convalescent Donor Plasma. Journal of Clinical Immunology, 1-3 (2021).
16. P. Bastard et al., Preexisting autoantibodies to type I IFNs underlie critical COVID-19 pneumonia in patients with APS-1. The Journal of Experimental Medicine 218, (2021).
17. P. Bastard et al., Auto-antibodies to type I IFNs can underlie adverse reactions to yellow fever live attenuated vaccine. The Journal of Experimental Medicine 218, (2021).
18. H. Hemmi et al., Small anti-viral compounds activate immune cells via the TLR7 MyD88-dependent signaling pathway. Nature Immunology 3, 196-200 (2002).
19. S. S. Diebold, T. Kaisho, H. Hemmi, S. Akira, C. Reis e Sousa, Innate antiviral responses by means of TLR7-mediated recognition of single-stranded RNA. Science (New York, N. Y.) 303, 1529-1531 (2004).
20. F. Heil et al., Species-specific recognition of single-stranded RNA via toll-like receptor 7 and 8. Science (New York, N.Y.) 303, 1526-1529 (2004).
21. J. M. Lund et al., Recognition of single-stranded RNA viruses by Toll-like receptor 7. Proceedings of the National Academy of Sciences of the United States of America 101, 5598-5603 (2004).
22. B. Beutler, Inferences, questions and possibilities in Toll-like receptor signalling. Nature 430, 257-263 (2004).
23. M. Colonna, G. Trinchieri, Y. J. Liu, Plasmacytoid dendritic cells in immunity. Nature Immunology 5, 1219-1226 (2004).
24. M. Gilliet, W. Cao, Y. J. Liu, Plasmacytoid dendritic cells: sensing nucleic acids in viral infection and autoimmune diseases. Nat Rev Immunol 8, 594-606 (2008).
25. B. Reizis, Plasmacytoid Dendritic Cells: Development, Regulation, and Function. Immunity 50, 37-50 (2019).
26. A. T. Bender et al., TLR7 and TLR8 Differentially Activate the IRF and NF-kappaB Pathways in Specific Cell Types to Promote Inflammation. ImmunoHorizons 4, 93-107 (2020).
27. K. Maeda, S. Akira, TLR7 Structure: Cut in Z-Loop. Immunity 45, 705-707 (2016).
28. J. Aluri et al., Immunodeficiency and bone marrow failure with mosaic and germline TLR8 gain of function. Blood 137, 2450-2462 (2021).
29. C. Uggenti, A. Lepelley, Y. J. Crow, Self-Awareness: Nucleic Acid-Driven Inflammation and the Type I Interferonopathies. Annual Review of Immunology 37, 247-267 (2019).
30. B. Boisson, J. L. Casanova, TLR8 gain of function: a tall surprise. Blood 137, 2420-2422 (2021).
31. M. Uhlen et al., A genome-wide transcriptomic analysis of protein-coding genes in human blood cells. Science (New York, N.Y.) 366, (2019).
32. C. Fallerini et al., Association of Toll-like receptor 7 variants with life-threatening COVID-19 disease in males: findings from a nested case-control study. eLife 10, (2021).
33. C. I. van der Made et al., Presence of Genetic Variants Among Young Men With Severe COVID-19. Jama 324, 663-673 (2020).
34. A. Belkadi et al., Whole-exome sequencing to analyze population structure, parental inbreeding, and familial linkage. Proceedings of the National Academy of Sciences of the United States of America 113, 6713-6718 (2016).
35. M. Shodell, F. P. Siegal, Circulating, interferon-producing plasmacytoid dendritic cells decline during human ageing. Scand J Immunol 56, 518-521 (2002).
36. Y. Jing et al., Aging is associated with a numerical and functional decline in plasmacytoid dendritic cells, whereas myeloid dendritic cells are relatively unaltered in human peripheral blood. Human immunology 70, 777-784 (2009).
37. M. V. Splunter et al., Plasmacytoid dendritic cell and myeloid dendritic cell function in ageing: A comparison between elderly and young adult women. PLoS One 14, e0225825 (2019).
38. N. V. Giltiay et al., Overexpression of TLR7 promotes cell-intrinsic expansion and autoantibody production by transitional T1 B cells. The Journal of Experimental Medicine 210, 2773-2789 (2013).
39. G. T. Consortium, The Genotype-Tissue Expression (GTEx) project. Nature Genetics 45, 580-585 (2013).
40. K. J. Travaglini et al., A molecular cell atlas of the human lung from single-cell RNA sequencing. Nature 587, 619-625 (2020).
41. M. Cella et al., Plasmacytoid monocytes migrate to inflamed lymph nodes and produce large amounts of type I interferon. Nature Medicine 5, 919-923 (1999).
42. F. P. Siegal et al., The nature of the principal type 1 interferon-producing cells in human blood. Science (New York, N.Y.) 284, 1835-1837 (1999).
43. K. Honda, T. Taniguchi, IRFs: master regulators of signalling by Toll-like receptors and cytosolic pattern-recognition receptors. Nature Reviews Immunology 6, 644-658 (2006).
44. F. Onodi et al., SARS-CoV-2 induces human plasmacytoid predendritic cell diversification via UNC93B and IRAK4. The Journal of Experimental Medicine 218, (2021).
45. L. Cervantes-Barragan et al., Plasmacytoid dendritic cells produce type I interferon and reduce viral replication in airway epithelial cells after SARS-CoV-2 infection. bioRxiv, 2021.2005.2012.443948 (2021).
46. S. G. Alculumbre et al., Diversification of human plasmacytoid predendritic cells in response to a single stimulus. Nature Immunology 19, 63-75 (2018).
47. H. von Bernuth et al., Pyogenic bacterial infections in humans with MyD88 deficiency. Science (New York, N.Y.) 321, 691-696 (2008).
48. C. Picard et al., Pyogenic bacterial infections in humans with IRAK-4 deficiency. Science (New York, N.Y.) 299, 2076-2079 (2003).
49. K. Yang et al., Human TLR-7-, -8-, and -9-mediated induction of IFN-alpha/beta and -lambda Is IRAK-4 dependent and redundant for protective immunity to viruses. Immunity 23, 465-478 (2005).
50. J. L. Casanova, L. Abel, L. Quintana-Murci, Human TLRs and IL-1Rs in host defense: natural insights from evolutionary, epidemiological, and clinical genetics. Annual Review of Immunology 29, 447-491 (2011).
51. B. Boisson, The genetic basis of pneumococcal and staphylococcal infections: inborn errors of human TLR and IL-1R immunity. Human Genetics 139, 981-991 (2020).
52. L. B. Barreiro et al., Evolutionary dynamics of human Toll-like receptors and their different contributions to host defense. PLoS Genetics 5, e1000562 (2009).
53. Y. J. Liu, Dendritic cell subsets and lineages, and their functions in innate and adaptive immunity. Cell 106, 259-262 (2001).
54. M. Swiecki, M. Colonna, Unraveling the functions of plasmacytoid dendritic cells during viral infections, autoimmunity, and tolerance. Immunological Reviews 234, 142-162 (2010).
55. F. J. Barrat, L. Su, A pathogenic role of plasmacytoid dendritic cells in autoimmunity and chronic viral infection. The Journal of Experimental Medicine 216, 1974-1985 (2019).
56. I. Sologuren et al., Lethal Influenza in Two Related Adults with Inherited GATA2 Deficiency. Journal of Clinical Immunology 38, 513-526 (2018).
57. D. C. Vinh et al., Autosomal dominant and sporadic monocytopenia with susceptibility to mycobacteria, fungi, papillomaviruses, and myelodysplasia. Blood 115, 1519-1529 (2010).
58. R. E. Dickinson et al., Exome sequencing identifies GATA-2 mutation as the cause of dendritic cell, monocyte, B and NK lymphoid deficiency. Blood 118, 2656-2658 (2011).
59. M. Pasquet et al., High frequency of GATA2 mutations in patients with mild chronic neutropenia evolving to MonoMac syndrome, myelodysplasia, and acute myeloid leukemia. Blood 121, 822-829 (2013).
60. V. Bigley, U. Cytlak, M. Collin, Human dendritic cell immunodeficiencies. Seminars in Cell and Developmental Biology 86, 50-61 (2019).
61. N. Kadowaki et al., Subsets of human dendritic cell precursors express different toll-like receptors and respond to different microbial antigens. The Journal of Experimental Medicine 194, 863-869 (2001).
62. K. Honda et al., Spatiotemporal regulation of MyD88-IRF-7 signalling for robust type-I interferon induction. Nature 434, 1035-1040 (2005).
63. D. Gao et al., TLR3 controls constitutive IFN-beta antiviral immunity in human fibroblasts and cortical neurons. Journal of Clinical Investigation 131, (2021).
64. Y. Wang, M. Swiecki, S. A. McCartney, M. Colonna, dsRNA sensors and plasmacytoid dendritic cells in host defense and autoimmunity. Immunological Reviews 243, 74-90 (2011).
65. M. A. DePristo et al., A framework for variation discovery and genotyping using next-generation DNA sequencing data. Nature Genetics 43, 491-498 (2011).
66. H. Li, R. Durbin, Fast and accurate short read alignment with Burrows-Wheeler transform. Bioinformatics (Oxford, England) 25, 1754-1760 (2009).
67. W. McLaren et al., The Ensembl Variant Effect Predictor. Genome Biol 17, 122 (2016).
68. C. L. Ku et al., Selective predisposition to bacterial infections in IRAK-4-deficient children: IRAK-4-dependent TLRs are otherwise redundant in protective immunity. The Journal of Experimental Medicine 204, 2407-2422 (2007).

This invention may be embodied in other forms or carried out in other ways without departing from the spirit or essential characteristics thereof. The present disclosure is therefore to be considered as in all aspects illustrated and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

1. A method for evaluating the presence of anti-type I IFN specific auto-antibodies (auto-Abs) in a patient or individual positive for or at risk for SARS-CoV-2 infection, having COVID-19 disease, prior to vaccination with live attenuated vaccine (LAV), or having vaccine-associated disease and thereby determining treatment and treating the patient or individual comprising:
(a) isolating a blood or serum sample from said patient or individual;
(b) evaluating the blood or serum sample for auto-antibodies specific for one or more type I IFN selected from:
(i) IFN-α2 and IFN-ω;
(ii) IFN-α2, IFN-ω and IFN-β;
(iii) IFN-α2, IFN-ω, IFN-β and IFN-ε;
(iv) IFN-α2, IFN-ω, IFN-β, IFN-ε and IFN-κ;
(v) IFN-α2, IFN-ω, IFN-β and IFN-α1/13;
(vi) IFN-α2, IFN-ω, IFN-β, IFN-α1/13 and IFN-α14;
(vii) IFN-α2, IFN-ω, IFN-β, IFN-α1/13, IFN-α14 and IFN-α7;
(viii) IFN-α2, IFN-ω, IFN-α1/13, IFN-α14, IFN-α7;
(ix) IFN-α2, IFN-ω, IFN-β, IFN-ε, IFN-α1, IFN-α2, IFN-α6, IFN-α13, IFN-α14 and IFN-α16; and
(x) IFN-α2, IFN-ω, IFN-α1, IFN-α2, IFN-α6, IFN-α13, IFN-α14 and IFN-α16;
(c) wherein a patient or individual having one or more auto-antibody is not administered the vaccine or wherein a patient or individual positive for or at risk for SARS-CoV-2 infection, having COVID-19 disease, or having vaccine-associated disease is treated to remove or deplete the auto-Abs and/or is administered a type I IFN against which they do not have auto-Abs and/or is administered an immune-modulatory agent that increases or facilitates type I IFN-mediated response.

2. The method of claim 1 for evaluating the presence of anti-type I IFN specific auto-antibodies (auto-Abs) in a patient or individual positive for or at risk for SARS-CoV-2 infection or having COVID-19 disease and thereby determining treatment and treating the patient or individual comprising:
(a) isolating a blood or serum sample from said patient or individual;
(b) evaluating the blood or serum sample for auto-antibodies specific for one or more type I IFN selected from:
(i) IFN-α2 and IFN-ω;
(ii) IFN-α2, IFN-ω and IFN-β;
(iii) IFN-α2, IFN-ω, IFN-β and IFN-ε;
(iv) IFN-α2, IFN-ω, IFN-β, IFN-ε and IFN-κ;
(v) IFN-α2, IFN-ω, IFN-β, IFN-ε, IFN-α1, IFN-α2, IFN-α6, IFN-α13, IFN-α14 and IFN-α16; and
(vi) IFN-α2, IFN-ω, IFN-α1, IFN-α2, IFN-α6, IFN-α13, IFN-α14 and IFN-α16;
(c) wherein a patient or individual having one or more auto-antibody is treated to remove or deplete the auto-Abs and/or is administered a type I IFN against which they do not have auto-Abs and/or is administered an immune-modulatory agent that increases or facilitates type I IFN-mediated response.

3. The method of claim 1 for evaluating the presence of anti-type I IFN specific auto-antibodies (auto-Abs) in a patient or individual prior to vaccination with live attenuated vaccine (LAV) against yellow fever virus or having yellow fever vaccine-associated disease and thereby determining whether the patient or individual can safely receive YFV LAV and/or treating the patient or individual comprising:
(a) isolating a blood or serum sample from said patient or individual;
(b) evaluating the blood or serum sample for auto-antibodies specific for one or more type I IFN selected from:
(i) IFN-α2 and IFN-ω;
(ii) IFN-α2, IFN-ω and IFN-β;
(iii) IFN-α2, IFN-ω, IFN-β and IFN-α1/13;
(iv) IFN-α2, IFN-ω, IFN-β, IFN-α1/13 and IFN-α14;
(v) IFN-α2, IFN-ω, IFN-β, IFN-α1/13, IFN-α14 and IFN-α7; and
(vi) IFN-α2, IFN-ω, IFN-α1/13, IFN-α14, IFN-α7;
(c) wherein a patient or individual having one or more auto-antibody is not administered the vaccine or wherein a patient or individual having yellow fever vaccine-associated disease is treated to remove or deplete the auto-Abs and/or is administered a type I IFN against which they do not have auto-Abs and/or is administered an immune-modulatory agent that increases or facilitates type I IFN-mediated response.

4. The method of claim 1, wherein the blood or serum sample is evaluated for neutralizing antibodies.

5. The method of claim 1, wherein plasmapheresis is conducted on the patient or individual to deplete the antibodies, or wherein B cells, such as auto-reactive B cells, and/or plasmacytes or plasmablasts are depleted in the patient or individual.

6. The method of claim 1, wherein a patient or individual having auto-Abs against one or more of IFN-α2, IFN-ω, IFN-ε, IFN-α1, IFN-α2, IFN-α6, IFN-α13, IFN-α14 or IFN-α16 and not having auto-Abs against IFN-β is treated by administering IFN-β.

7. The method of claim 1, wherein a patient or individual not having auto-Abs against IFN-β, or having auto-Abs against one or more of IFN-α2, IFN-ω, IFN-ε, IFN-α1/13, IFN-α14, or IFN-α7 and not having auto-Abs against IFN-β, is treated by administering IFN-0.

8. The method of claim 1, wherein a patient or individual having auto-Abs against one or more Type I IFN selected from IFN-ω, IFN-ε, IFN-β, IFN-κ, and not having auto-Abs against IFN-α2 is treated by administering IFN-α2.

9. The method of claim 1, wherein the blood or serum sample is additionally or alternatively evaluated for auto-antibodies specific for one or more type I IFN selected from IFN-α4, IFN-α5, IFN-α6, IFN-α8, IFN-α10, IFN-α16, IFN-α17 and IFN-α21.

10. The method of claim 9, wherein the blood or serum sample is additionally or alternatively evaluated for auto-antibodies specific for one or more type I IFN selected from IFN-κ and IFN-ε.

11. (canceled)

12. The method of claim 1, wherein a patient or individual having auto-Abs against one or more Type I IFN is treated by administering an IFN subtype or an IFN-α subtype which is not neutralized by the patient's or individual's auto-Abs.

13. (canceled)

14. The method of claim 1, wherein the LAV is a COVID-19/SARsCoV-2 vaccine or is a yellow fever vaccine, or wherein the vaccine-associated disease is COVID-19/SARs CoV-2 vaccine-associated disease or is yellow fever virus (YFV) vaccine-associated disease.

15. (canceled)

16. An assay for evaluating the presence of auto-antibodies directed against one or more Type I IFN in a patient or individual positive for or at risk for SARS-CoV-2 infection or having COVID-19 disease or prior to vaccination with live attenuated vaccine (LAV) or having vaccine-associated disease comprising:
(a) contacting a sample of blood or serum from the patient or individual with one or more recombinant type I IFN protein selected from IFN-α2, IFN-ω, IFN-β, IFN-ε and IFN-κ, wherein each IFN protein is labeled with a distinct detectable tag or marker to form an antibody-protein complex;
(b) contacting any antibody-protein complex of (a) with one or more labeled anti-human immune globulin molecule that will bind and label auto-antibody bound to any one or more IFN protein;
(c) specifically and selectively detecting each type I IFN protein bound by specific auto-Ab thereto.

17. The assay of claim 16, wherein one or more recombinant type I IFN protein is labeled with a fluorescent marker, wherein one or more recombinant type I IFN protein is covalently coupled to a magnetic bead with a fluorescent marker, or wherein each of the one or more recombinant type I IFN proteins is covalently coupled to a magnetic bead with a distinct and differential fluorescent marker.

18. (canceled)

19. (canceled)

20. The assay of claim 16, wherein the one or more labeled anti-human immune globulin molecule that will bind and label bound auto-antibody is a labeled non-human animal derived anti-human IgG.

21. The assay of claim 16, wherein the one or more recombinant type I IFN protein is selected from IFN-α2, IFN-ω and IFN-β, or wherein the one or more recombinant type I IFN protein is selected from IFN-α2 and IFN-ω.

22. (canceled)

23. (canceled)

24. A kit for for evaluating the presence of auto-antibodies directed against one or more Type I IFN in a patient or individual at risk of, suspected of or determined to have coronavirus or in a patient or individual prior to vaccination with a live attenuated vaccine (LAV) or having vaccine-associated disease comprising:
(a) one or more recombinant type I IFN protein selected from IFN-α2, IFN-ω, IFN-β, IFN-ε and IFN-κ, wherein each IFN protein is labeled with a distinct detectable tag or marker;
(b) one or more labeled anti-human immune globulin molecule that will bind and label auto-antibody bound to any one or more IFN protein;
(c) a means for specific and selective detection of each type I IFN protein bound by specific auto-Ab thereto.

25. The kit of claim 24, wherein one or more recombinant type I IFN protein is labeled with a fluorescent marker, wherein one or more recombinant type I IFN protein is covalently coupled to a magnetic bead with a fluorescent marker, or wherein each of the one or more recombinant type I IFN proteins is covalently coupled to a magnetic bead with a distinct and differential fluorescent marker.

26. (canceled)

27. (canceled)

28. The kit of claim 24, wherein the one or more labeled anti-human immune globulin molecule that will bind and label bound auto-antibody is a labeled non-human animal derived anti-human IgG.

29. The kit of claim 24, wherein the one or more recombinant type I IFN protein is selected from IFN-α2, IFN-ω and IFN-β, or wherein the one or more recombinant type I IFN protein is selected from IFN-α2 and IFN-ω.

30. (canceled)

31. The kit of claim 24, wherein the LAV is a COVID-19/SARsCoV-2 vaccine or is a yellow fever vaccine, or wherein the vaccine-associated disease is COVID-19/SARsCoV-2 vaccine-associated disease or is yellow fever virus (YFV) vaccine-associated disease.

32. (canceled)

33. A method for evaluating a patient or individual positive for or at risk for SARS-CoV-2 infection or having COVID-19 disease or prior to vaccination with live attenuated vaccine or having vaccine-associated disease for the presence of an autosomal recessive IFN deficiency or loss-of-function (LOF) variation or mutation in a type I IFN pathway or response relevant gene and thereby determining treatment or whether the patient or individual can be safely vaccinated or should be vaccinated and/or treating the patient or individual comprising:
(a) isolating a blood or serum sample from said patient or individual;
(b) evaluating the blood or serum sample for a LOF variation or mutation in one or more type I IFN pathway gene selected from:
(i) TLR7, IFNAR1, IRF7, IFIH1, TLR3, TBK1, IRF3, TICAM1, UNC93B1, IFNAR2, STAT1, STAT2, TRAF3 or IRF9;
(ii) TLR7, IFNAR1, IRF7, IFIH1, TLR3, TBK1, IRF3, TICAM1, UNC93B1, IFNAR2, STAT1, STAT2 or TRAF3;
(iii) TLR7, IFNAR1, IRF7, IFIH1, TLR3, TBK1, IRF3, TICAM1, UNC93B1 and IFNAR2;
(iv) TLR7, IFNAR1, IRF7, IFIH1, TLR3, TBK1, IRF3, TICAM1 and UNC93B1; and
(v) IFNAR1, IRF7, IFIH1, TLR3, TBK1, IRF3, TICAM1 and UNC93B1
(c) wherein a patient or individual determined to have an autosomal recessive IFN deficiency or LOF mutation is administered one or more Type I IFN to replace the function lost due to the mutation and/or is administered an immune-modulatory agent that increases or facilitates type I IFN-mediated response.

34. The method of claim 33, wherein the patient or individual determined to have a LOF mutation is administered a Type I IFN, or wherein the patient or individual determined to have a LOF mutation is administered IFN-α2 or IFN-β.

35. (canceled)

36. The method of claim 33, wherein the variation or mutation in one or more type I IFN pathway gene is selected from a mutation provided in TABLE 1, TABLE 2, TABLE 11 or TABLE 15.

37. (canceled)

38. The method of claim 36, wherein the variation or mutation in one or more type I IFN pathway gene selected from TABLE 1, TABLE 2, TABLE 11 or TABLE 15 is determined via whole genome or whole exome sequencing, or is determined via a detectably labeled primer or oligonucleotide specific for the mutation, wherein hybridization and detection of the labeled primer or oligonucleotide is diagnostic for the presence of the mutation and LOF of the type I IFN in the patient or individual.

39. (canceled)

40. (canceled)

41. The method of claim 33, wherein the LAV is a COVID-19/SARsCoV-2 vaccine or is a yellow fever vaccine, or wherein the vaccine-associated disease is COVID-19/SARsCoV-2 vaccine-associated disease or is yellow fever virus (YFV) vaccine-associated disease.

42. (canceled)

43. A method for evaluating a patient or individual positive for or at risk for SARS-CoV-2 infection or having COVID-19 disease for response to SARS-CoV-2 infection comprising:
(a) isolating plasmacytoid dendritic cells (pDCs) from the patient or individual;
(b) contacting the isolated pDCs with SARS-CoV-2 virus; and
(c) assessing the production of type I IFNs by the pDCs in response to SARS-CoV-2;
wherein reduced production of type I IFNs indicates that the patient or individual is altered in response to virus and is at high risk for severe COVID-19 disease.

44. The method of claim 43, wherein the method further includes thereby determining treatment and treating the patient or individual, and wherein the patient or individual is administered the type I IFN or one or more type I IFN for which production is reduced.

45. (canceled)

46. The method of claim 43, wherein the patient is suspected of or first determined to carry or have family history of a variation or mutation in one or more type I IFN pathway or response relevant gene selected from TABLE 1, TABLE 2, TABLE 11 or TABLE 15.
